(19) United States
(12) Patent Application Publication
(10) Pub. No.: US 2004/0029222 A1
(43) Pub. Date: Feb. 12, 2004

(54) Proteins and nucleic acids encoding same

(76) Inventors: Shlomit R. Edinger, New Haven, CT (US); John R. MacDougall, Hamden, CT (US); Isabelle Millet, Milford, CT (US); Karen Ellerman, Branford, CT (US); David J. Stone, Guilford, CT (US); Valerie Gerlach, Branford, CT (US); William M. Grosse, Branford, CT (US); John P. Alsobrook II, Madison, CT (US); Denise M. Lepley, Branford, CT (US); Danier K. Rieger, Branford, CT (US); Catherine E. Burgess, Wethersfield, CT (US); Stacie J. Casman, North Haven, CT (US); Kimberly A. Spytek, New Haven, CT (US); Ference L. Boldog, North Haven, CT (US); Li Li, Branford, CT (US); Muralidhara Padigaru, Branford, CT (US); Vishnu Mishra, Gainesville, FL (US); Meera Patturajan, Branford, CT (US); Suresh G. Shenoy, Branford, CT (US); Luca Rastelli, Guilford, CT (US); Velizar T. Tchernev, Branford, CT (US); Corine A.M. Vernet, Branford, CT (US); Bryan D. Zerhusen, Branford, CT (US); Uriel M. Malyankar, Branford, CT (US); Xiaojia (Sasha) Guo, Branford, CT (US); Charles E. Miller, Guilford, CT (US); Esha A. Gangolli, Madison, CT (US); Michael Grosse, (US)

Correspondence Address: MINTZ, LEVIN, COHN, FERRIS,, GLOVSKY AND POPEO, P.C., One Financial Center, Boston, MA, 02111, US

(21) Appl. No.: 10/218,779

(22) Filed: Aug. 14, 2002

Related U.S. Application Data
(63) Continuation of application No. 09/995,514, filed on Nov. 28, 2001, now abandoned.
(60) Provisional application No. 60/253,834, filed on Nov. 29, 2000.
(60) Provisional application No. 60/250,926, filed on Nov. 30, 2000.
(60) Provisional application No. 60/264,180, filed on Jan. 25, 2001.
(60) Provisional application No. 60/313,656, filed on Aug. 20, 2001.
(60) Provisional application No. 60/327,456, filed on Oct. 5, 2001.

(51) Int. Cl. C12Q 1/68; G01N 33/574; C07H 21/04; C12N 9/00; C12P 21/02; C12N 5/06; C07K 14/47; C07K 16/30

(52) U.S. Cl. 435/69.1; 435/183; 435/320.1; 435/325; 530/350; 536/23.2; 530/388.1; 435/7.23; 435/6

(57) ABSTRACT

Disclosed herein are nucleic acid sequences that encode novel polypeptides. Also disclosed are polypeptides encoded by these nucleic acid sequences, and antibodies, which immunospecifically-bind to the polypeptide, as well as derivatives, variants, mutants, or fragments of the aforementioned polypeptide, polynucleotide, or antibody. The invention further discloses therapeutic, diagnostic and research methods for diagnosis, treatment, and prevention of disorders involving any one of these novel human nucleic acids and proteins.

### RELATED APPLICATIONS

This application claims priority to U.S. Ser. No. 09/995,514, filed Nov. 28, 2001, pending, which claims the benefit of U.S. Ser. No. 60/253,834, filed Nov. 29, 2000, abandoned; U.S. Ser. No. 60/250,926, filed Nov. 30, 2000, abandoned; U.S. Ser. No. 60/264,180, filed Jan. 25, 2001, abandoned; U.S. Ser. No. 60/313,656, filed Aug. 20, 2001, pending; and U.S. Ser. No. 60/327,456, filed Oct. 5, 2001, pending; each of which is incorporated by reference in its entirety.

### FIELD OF THE INVENTION

The invention generally relates to nucleic acids and polypeptides encoded thereby.

### BACKGROUND OF THE INVENTION

The invention generally relates to nucleic acids and polypeptides encoded therefrom. More specifically, the invention relates to nucleic acids encoding cytoplasmic, nuclear, membrane bound, and secreted polypeptides, as well as vectors, host cells, antibodies, and recombinant methods for producing these nucleic acids and polypeptides.

### SUMMARY OF THE INVENTION

The invention is based in part upon the discovery of nucleic acid sequences encoding novel polypeptides. The novel nucleic acids and polypeptides are referred to herein as NOVX, or NOV1, NOV2, NOV3, NOV4, NOV5, NOV6, NOV7, NOV8, NOV9, NOV10, NOV11, and NOV12 nucleic acids and polypeptides. These nucleic acids and polypeptides, as well as derivatives, homologs, analogs and fragments thereof, will hereinafter be collectively designated as “NOVX” nucleic acid or polypeptide sequences.

In one aspect, the invention provides an isolated NOVX nucleic acid molecule encoding a NOVX polypeptide that includes a nucleic acid sequence that has identity to the nucleic acids disclosed in SEQ ID NOS: 1, 3, 5, 7, 9, 11, 13, 15, 17, 19, 21, 23, 25, 27, 29, and 31. In some embodiments, the NOVX nucleic acid molecule will hybridize under stringent conditions to a nucleic acid sequence complementary to a nucleic acid molecule that includes a protein-coding sequence of a NOVX nucleic acid sequence. The invention also includes an isolated nucleic acid that encodes a NOVX polypeptide, or a fragment, homolog, analog or derivative thereof. For example, the nucleic acid can encode a polypeptide at least 80% identical to a polypeptide comprising the amino acid sequences of SEQ ID NOS: 2, 4, 6, 8, 10, 12, 14, 16, 18, 20, 22, 24, 26, 28, 30, and 32. The nucleic acid can be, for example, a genomic DNA fragment or a cDNA molecule that includes the nucleic acid sequence of any of SEQ ID NOS: 1, 3, 5, 7, 9, 11, 13, 15, 17, 19, 21, 23, 25, 27, 29,and31.

Also included in the invention is an oligonucleotide, e.g., an oligonucleotide which includes at least 6 contiguous nucleotides of a NOVX nucleic acid (e.g., SEQ ID NOS: 1, 3, 5, 7, 9, 11, 13, 15, 17, 19, 21, 23, 25, 27, 29, and 31) or a complement of said oligonucleotide.

Also included in the invention are substantially purified NOVX polypeptides (SEQ ID NOS: 2, 4, 6, 8, 10, 12, 14, 16, 18, 20, 22, 24, 26, 28, 30, and 32). In certain embodiments, the NOVX polypeptides include an amino acid sequence that is substantially identical to the amino acid sequence of a human NOVX polypeptide.

The invention also features antibodies that immunoselectively bind to NOVX polypeptides, or fragments, homologs, analogs or derivatives thereof.

In another aspect, the invention includes pharmaceutical compositions that include therapeutically- or prophylactically-effective amounts of a therapeutic and a pharmaceutically-acceptable carrier. The therapeutic can be, e.g., a NOVX nucleic acid, a NOVX polypeptide, or an antibody specific for a NOVX polypeptide. In a further aspect, the invention includes, in one or more containers, a therapeutically- or prophylactically-effective amount of this pharmaceutical composition.

In a further aspect, the invention includes a method of producing a polypeptide by culturing a cell that includes a NOVX nucleic acid, under conditions allowing for expression of the NOVX polypeptide encoded by the DNA. If desired, the NOVX polypeptide can then be recovered.

In another aspect, the invention includes a method of detecting the presence of a NOVX polypeptide in a sample. In the method, a sample is contacted with a compound that selectively binds to the polypeptide under conditions allowing for formation of a complex between the polypeptide and the compound. The complex is detected, if present, thereby identifying the NOVX polypeptide within the sample.

The invention also includes methods to identify specific cell or tissue types based on their expression of a NOVX.

Also included in the invention is a method of detecting the presence of a NOVX nucleic acid molecule in a sample by contacting the sample with a NOVX nucleic acid probe or primer, and detecting whether the nucleic acid probe or primer bound to a NOVX nucleic acid molecule in the sample.

In a further aspect, the invention provides a method for modulating the activity of a NOVX polypeptide by contacting a cell sample that includes the NOVX polypeptide with a compound that binds to the NOVX polypeptide in an amount sufficient to modulate the activity of said polypeptide. The compound can be, e.g., a small molecule, such as a nucleic acid, peptide, polypeptide, peptidomimetic, carbohydrate, lipid or other organic (carbon containing) or inorganic molecule, as further described herein.

Also within the scope of the invention is the use of a therapeutic in the manufacture of a medicament for treating or preventing disorders or syndromes including, e.g., cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, hypercoagulation, hemophilia, idiopathic thrombocytopenic purpura, heart failure, secondary pathologies caused by heart failure and hypertension, hypotension, angina pectoris, myocardial infarction, tuberous sclerosis, scleroderma, transplantation, autoimmune disease, lupus erythematosus, viral/bacterial/parasitic infections, multiple sclerosis, autoimmume disease, allergies, immunodeficiencies, graft versus host disease, asthma, emphysema, ARDS, inflammation and modulation of the immune response, viral pathogenesis, aging-related disorders, Th1 inflammatory diseases such as rheumatoid arthritis, multiple sclerosis, inflammatory bowel diseases, AIDS, wound repair, obesity, diabetes, endocrine disorders, anorexia, bulimia, renal artery stenosis, interstitial nephritis, glomerulonephritis, polycystic kidney disease, systemic, renal tubular acidosis, IgA nephropathy, nephrological disesases, hypercalceimia, Lesch-Nyhan syndrome, Von Hippel-Lindau (VHL) syndrome, trauma, regeneration (in vitro and in vivo), Hirschsprung's disease , Crohn's Disease, appendicitis, endometriosis, laryngitis, psoriasis, actinic keratosis, acne, hair growth/loss, allopecia, pigmentation disorders, myasthenia gravis, alpha-mannosidosis, beta-mannosidosis, other storage disorders, peroxisomal disorders such as zellweger syndrome, infantile refsum disease, rhizomelic chondrodysplasia (chondrodysplasia punctata, rhizomelic), and hyperpipecolic acidemia, osteoporosis, muscle disorders, urinary retention, Albright Hereditary Ostoeodystrophy, ulcers, Alzheimer's disease, stroke, Parkinson's disease, Huntington's disease, cerebral palsy, epilepsy, Lesch-Nyhan syndrome, multiple sclerosis, ataxia-telangiectasia, behavioral disorders, addiction, anxiety, pain, neuroprotection, Stroke, Aphakia, neurodegenerative disorders, neurologic disorders, developmental defects, conditions associated with the role of GRK2 in brain and in the regulation of chemokine receptors, encephalomyelitis, anxiety, schizophrenia, manic depression, delirium, dementia, severe mental retardation and dyskinesias, Gilles de la Tourette syndrome, leukodystrophies, cancers, breast cancer, CNS cancer, colon cancer, gastric cancer, lung cancer, melanoma, ovarian cancer, pancreatic cancer, kidney cancer, colon cancer, prostate cancer, neuroblastoma, and cervical cancer, Neoplasm; adenocarcinoma, lymphoma; uterus cancer, benign prostatic hypertrophy, fertility, control of growth and development/differentiation related functions such as but not limited maturation, lactation and puberty, reproductive malfunction, and/or other pathologies and disorders of the like.

The therapeutic can be, e.g., a NOVX nucleic acid, a NOVX polypeptide, or a NOVX-specific antibody, or biologically-active derivatives or fragments thereof.

For example, the compositions of the present invention will have efficacy for treatment of patients suffering from the diseases and disorders disclosed above and/or other pathologies and disorders of the like. The polypeptides can be used as immunogens to produce antibodies specific for the invention, and as vaccines. They can also be used to screen for potential agonist and antagonist compounds. For example, a cDNA encoding NOVX may be useful in gene therapy, and NOVX may be useful when administered to a subject in need thereof. By way of non-limiting example, the compositions of the present invention will have efficacy for treatment of patients suffering from the diseases and disorders disclosed above and/or other pathologies and disorders of the like.

The invention further includes a method for screening for a modulator of disorders or syndromes including, e.g., the diseases and disorders disclosed above and/or other pathologies and disorders of the like. The method includes contacting a test compound with a NOVX polypeptide and determining if the test compound binds to said NOVX polypeptide. Binding of the test compound to the NOVX polypeptide indicates the test compound is a modulator of activity, or of latency or predisposition to the aforementioned disorders or syndromes.

Also within the scope of the invention is a method for screening for a modulator of activity, or of latency or predisposition to disorders or syndromes including, e.g., the diseases and disorders disclosed above and/or other pathologies and disorders of the like by administering a test compound to a test animal at increased risk for the aforementioned disorders or syndromes. The test animal expresses a recombinant polypeptide encoded by a NOVX nucleic acid. Expression or activity of NOVX polypeptide is then measured in the test animal, as is expression or activity of the protein in a control animal which recombinantly-expresses NOVX polypeptide and is not at increased risk for the disorder or syndrome. Next, the expression of NOVX polypeptide in both the test animal and the control animal is compared. A change in the activity of NOVX polypeptide in the test animal relative to the control animal indicates the test compound is a modulator of latency of the disorder or syndrome.

In yet another aspect, the invention includes a method for determining the presence of or predisposition to a disease associated with altered levels of a NOVX polypeptide, a NOVX nucleic acid, or both, in a subject (e.g., a human subject). The method includes measuring the amount of the NOVX polypeptide in a test sample from the subject and comparing the amount of the polypeptide in the test sample to the amount of the NOVX polypeptide present in a control sample. An alteration in the level of the NOVX polypeptide in the test sample as compared to the control sample indicates the presence of or predisposition to a disease in the subject. Preferably, the predisposition includes, e.g., the diseases and disorders disclosed above and/or other pathologies and disorders of the like. Also, the expression levels of the new polypeptides of the invention can be used in a method to screen for various cancers as well as to determine the stage of cancers.

In a further aspect, the invention includes a method of treating or preventing a pathological condition associated with a disorder in a mammal by administering to the subject a NOVX polypeptide, a NOVX nucleic acid, or a NOVX-specific antibody to a subject (e.g., a human subject), in an amount sufficient to alleviate or prevent the pathological condition. In preferred embodiments, the disorder, includes, e.g., the diseases and disorders disclosed above and/or other pathologies and disorders of the like.

In yet another aspect, the invention can be used in a method to identity the cellular receptors and downstream effectors of the invention by any one of a number of techniques commonly employed in the art. These include but are not limited to the two-hybrid system, affinity purification, co-precipitation with antibodies or other specific-interacting molecules.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims.

### DETAILED DESCRIPTION OF THE INVENTION

The present invention provides novel nucleotides and polypeptides encoded thereby. Included in the invention are the novel nucleic acid sequences and their encoded polypeptides. The sequences are collectively referred to herein as “NOVX nucleic acids” or “NOVX polynucleotides” and the corresponding encoded polypeptides are referred to as “NOVX polypeptides” or “NOVX proteins.” Unless indicated otherwise, “NOVX” is meant to refer to any of the novel sequences disclosed herein. Table A provides a summary of the NOVX nucleic acids and their encoded polypeptides.

TABLE A
Sequences and Corresponding SEQ ID Numbers

| | | SEQ ID | | |
|---|---|---|---|---|
| | | NO | | |
| NOVX | | (nucleic | SEQ ID NO | |
| Assignment | Internal Identification | acid) | (polypeptide) | Homology |
| 1a | GMba58o1_A_da1 | 1 | 2 | Transmembrane receptor |
| | | | | UNC5H2-like |
| 1b | Gmba58o1_A | 3 | 4 | Transmembrane receptor |
| | | | | UNC5H2-like |
| 2a | SC126422078_A | 5 | 6 | Tyrosine Phosphatase |
| | | | | Precursor-like |
| 2b | CG50718-02 | 7 | 8 | Glomerular Mesangial Cell |
| | | | | Receptor Protein Tyrosine |
| | | | | Phosphatase Precursor like |
| 2c | CG50718-05 | 9 | 10 | Glomerular Mesangial Cell |
| | | | | Receptor Protein Tyrosine |
| | | | | Phosphatase Precursor like |
| 3 | 134899552_EXT | 11 | 12 | Human homolog of the |
| | | | | Drosophila pecanex-like |
| 4 | SC140515441_A | 13 | 14 | Aurora-related kinase 1- |
| | | | | like |
| 5 | SC44326718_A | 15 | 16 | 26S protease regulatory |
| | | | | subunit 4-like |
| 6 | GMAC073364_Ada1 | 17 | 18 | Mitsugumin29-like |
| 7 | 106973211_EXT | 19 | 20 | Wnt-15-like |
| 8 | 88091010-EXT | 21 | 22 | Wnt-14-like |
| 9 | AC069250_28_da1 | 23 | 24 | Beta-adrenergic receptor |
| | | | | kinase-like |
| 10 | AC058790_da25 | 25 | 26 | Alpha-mannosidase-like |
| 11a | GM57107065_da1 | 27 | 28 | C1q-related factor-like |
| 11b | CG54503-02 | 29 | 30 | C1q-related factor-like |
| 12 | SC132340676_A | 31 | 32 | Plexin 1-like |

NOVX nucleic acids and their encoded polypeptides are useful in a variety of applications and contexts. The various NOVX nucleic acids and polypeptides according to the invention are useful as novel members of the protein families according to the presence of domains and sequence relatedness to previously described proteins. Additionally, NOVX nucleic acids and polypeptides can also be used to identify proteins that are members of the family to which the NOVX polypeptides belong.

NOV1 is homologous to the transmembrane receptor UNC5H2-like family of proteins. Thus, NOV1 nucleic acids and polypeptides, antibodies and related compounds according to the invention will be useful in therapeutic and diagnostic applications implicated in, for example; cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, obesity, transplantation, diabetes, autoimmune disease, renal artery stenosis, interstitial nephritis, glomerulonephritis, polycystic kidney disease, systemic lupus erythematosus, renal tubular acidosis, IgA nephropathy, hypercalceimia, Lesch-Nyhan syndrome, Von Hippel-Lindau (VHL) syndrome, Alzheimer's disease, stroke, tuberous sclerosis, Parkinson's disease, Huntington's disease, cerebral palsy, epilepsy, Lesch-Nyhan syndrome, multiple sclerosis, ataxia-telangiectasia, leukodystrophies, behavioral disorders, addiction, anxiety, pain, neuroprotection, cancers, and/or other pathologies and disorders. Also since this gene is expressed at a measurably higher level in several cancer cell lines (including breast cancer, CNS cancer, colon cancer, gastric cancer, lung cancer, melanoma, ovarian cancer and pancreatic cancer), it may be useful in diagnosis and treatment of these cancers.

NOV2 is homologous to the protein tyrosine phosphatase precursor-like family of proteins. Thus NOV2 nucleic acids, polypeptides, antibodies and related compounds according to the invention will be useful in therapeutic and diagnostic applications implicated in, for example; cancer, kidney cancer, trauma, regeneration (in vitro and in vivo), viral/bacterial/parasitic infections, nephrological disesases including diabetes, autoimmune disease, renal artery stenosis, interstitial nephritis, glomerulonephritis, polycystic kidney disease, systemic lupus erythematosus, renal tubular acidosis, IgA nephropathy, hypercalceimia, Lesch-Nyhan syndrome, Hirschsprung's disease, Crohn's Disease, appendicitis, and/or other pathologies and disorders.

NOV3 is homologous to the Human homolog of the Drosophila pecanex family of proteins. Thus NOV3 nucleic acids, polypeptides, antibodies and related compounds according to the invention will be useful in therapeutic and diagnostic applications implicated in, for example; cancer,trauma, regeneration (in vitro and in vivo), viral/bacterial/parasitic infections, cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, multiple sclerosis, scleroderma, obesity, endometriosis, fertility, hypercoagulation, autoimmume disease, allergies, immunodeficiencies, transplantation, hemophilia, idiopathic thrombocytopenic purpura, graft versus host disease, Von Hippel- Lindau (VHL) syndrome, Alzheimer's disease, stroke, hypercalceimia, Parkinson's disease, Huntington's disease, cerebral palsy, epilepsy, ataxia-telangiectasia, leukodystrophies, behavioral disorders, addiction, anxiety, pain, neuroprotection, systemic lupus erythematosus, asthma, emphysema, ARDS, laryngitis, psoriasis, actinic keratosis, acne, hair growth/loss, allopecia, pigmentation disorders, endocrine disorders, diabetes, renal artery stenosis, interstitial nephritis, glomerulonephritis, polycystic kidney disease, systemic lupus erythematosus, renal tubular acidosis, IgA nephropathy, Lesch-Nyhan syndrome, and a variety of kidney diseases and/or other pathologies and disorders.

NOV4 is homologous to a family of Aurora-related kinase 1-like proteins. Thus, the NOV4 nucleic acids and polypeptides, antibodies and related compounds according to the invention will be useful in therapeutic and diagnostic applications implicated in, for example: breast, ovarian, colon, prostate, neuroblastoma, and cervical cancer, Cardiomyopathy, Atherosclerosis, Hypertension, Congenital heart defects, Aortic stenosis, Atrial septal defect (ASD), Atrioventricular (A-V) canal defect, Ductus arteriosus, Pulmonary stenosis, Subaortic stenosis, Ventricular septal defect (VSD), valve diseases, Tuberous sclerosis, Scleroderma, Obesity, Transplantation, Diabetes, Von Hippel-Lindau (VHL) syndrome, Pancreatitis, Alzheimer's disease, Stroke, hypercalceimia, Parkinson's disease, Huntington's disease, Cerebral palsy, Epilepsy, Lesch-Nyhan syndrome, Multiple sclerosis, Ataxia-telangiectasia, Leukodystrophies, Behavioral disorders, Addiction, Anxiety, Pain, and Neuroprotection, and/or other pathologies.

NOV5 is homologous to the 26S protease regulatory subunit 4-like family of proteins. Thus, NOV5 nucleic acids, polypeptides, antibodies and related compounds according to the invention will be useful in therapeutic and diagnostic applications implicated in, for example: cataract and Aphakia, Alzheimer's disease, neurodegenerative disorders, inflammation and modulation of the immune response, viral pathogenesis, aging-related disorders, neurologic disorders, cancer, and/or other pathologies.

NOV6 is homologous to the MITSUGUMIN29-like family of proteins. Thus, NOV6 nucleic acids, polypeptides, antibodies and related compounds according to the invention will be useful in therapeutic and diagnostic applications implicated in, for example: muscular dystrophy, Lesch-Nyhan syndrome, myasthenia gravis, diabetes, autoimmune disease, renal artery stenosis, interstitial nephritis, glomerulonephritis, polycystic kidney disease, systemic lupus erythematosus, renal tubular acidosis, IgA nephropathy, hypercalceimia, cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, obesity, transplantation, adrenoleukodystrophy, congenital adrenal hyperplasia, and other diseases, disorders and conditions of the like. Also since the invention is highly expressed in one of the lung cancer cell lines (Lung cancer NCI-H522 ), it may be useful in diagnosis and treatment of this cancer.

NOV7 is homologous to the Wnt-15-like family of proteins. Thus NOV7 nucleic acids, polypeptides, antibodies and related compounds according to the invention will be useful in Von Hippel-Lindau (VHL) syndrome, Alzheimer's disease, stroke, tuberous sclerosis, hypercalceimia, Parkinson's disease, Huntington's disease, cerebral palsy, epilepsy, Lesch-Nyhan syndrome, multiple sclerosis, ataxia-telangiectasia, leukodystrophies, behavioral disorders, addiction, anxiety, pain, neurodegeneration, cancer, developmental defects, and/or other pathologies/disorders.

NOV8 is homologous to members of the Wnt-14-like family of proteins. Thus, the NOV8 nucleic acids, polypeptides, antibodies and related compounds according to the invention will be useful in therapeutic and diagnostic applications implicated in, for example; Von Hippel-Lindau (VHL) syndrome, Alzheimer's disease, stroke, tuberous sclerosis, hypercalceimia, Parkinson's disease, Huntington's disease, cerebral palsy, epilepsy, Lesch-Nyhan syndrome, multiple sclerosis, ataxia-telangiectasia, leukodystrophies, behavioral disorders, addiction, anxiety, pain, neurodegeneration, cancer, developmental defects, and/or other pathologies/disorders.

NOV9 is homologous to the beta adrenergic receptor kinase-like family of proteins. Thus, NOV9 nucleic acids and polypeptides, antibodies and related compounds according to the invention will be useful in therapeutic and diagnostic applications implicated in, for example: heart failure, hypertension, secondary pathologies caused by heart failure and hypertension, and other diseases, disorders and conditions of the like. Additionally, the compositions of the present invention may have efficacy for treatment of patients suffering from conditions associated with the role of GRK2 in brain and in the regulation of chemokine receptors.

NOV10 is homologous to the alpha-mannosidase-like family of proteins. Thus, NOV10 nucleic acids and polypeptides, antibodies and related compounds according to the invention will be useful in therapeutic and diagnostic applications implicated in, for example: alpha-mannosidosis, beta-mannosidosis, other storage disorders, peroxisomal disorders such as zellweger syndrome, infantile refsum disease, rhizomelic chondrodysplasia (chondrodysplasia punctata, rhizomelic), and hyperpipecolic acidemia and other diseases, disorders and conditions of the like, and/or other pathologies/disorders.

NOV11 is homologous to the C1q-related factor-like family of proteins. Thus, NOV11 nucleic acids and polypeptides, antibodies and related compounds according to the invention will be useful in therapeutic and diagnostic applications implicated in, for example: Th1 inflammatory diseases such as rheumatoid arthritis, multiple sclerosis, inflammatory bowel diseases and psoriasis, lupus erythematosus and glomerulonephritis, control of growh and development/differentiation related functions such as but not limited maturation, lactation and puberty, osteoporosis, obesity, aging and reproductive malfunction and hence could be used in treatment and/or diagnosis of these disorders.

NOV12 is homologous to the Plexin-1 like family of proteins. Thus, NOV12 nucleic acids and polypeptides, antibodies, and related compounds according to the invention will be useful in therapeutic and diagnostic applications implicated in, for example: AIDS, cancer therapy, treatment of Neurologic diseases, Brain and/or autoimmune disorders like encephalomyelitis, neurodegenerative disorders, Alzheimer's Disease, Parkinson's Disorder, immune disorders, and hematopoietic disorders, endocrine diseases, muscle disorders, inflammation and wound repair, bacterial, fungal, protozoal and viral infections (particularly infections caused by HIV-1 or HIV-2), pain, cancer (including but not limited to Neoplasm; adenocarcinoma; lymphoma; prostate cancer; uterus cancer), anorexia, bulimia, asthma, Parkinson's disease, acute heart failure, hypotension, hypertension, urinary retention, osteoporosis, Crohn's disease; multiple sclerosis; and Treatment of Albright Hereditary Ostoeodystrophy, angina pectoris, myocardial infarction, ulcers, asthma, allergies, benign prostatic hypertrophy, and psychotic and neurological disorders, including anxiety, schizophrenia, manic depression, delirium, dementia, severe mental retardation and dyskinesias, such as Huntington's disease or Gilles de la Tourette syndrome, and/or other pathologies/disorders.

The NOVX nucleic acids and polypeptides can also be used to screen for molecules, which inhibit or enhance NOVX activity or function. Specifically, the nucleic acids and polypeptides according to the invention may be used as targets for the identification of small molecules that modulate or inhibit, e.g., neurogenesis, cell differentiation, cell proliferation, hematopoiesis, wound healing and angiogenesis.

Additional utilities for the NOVX nucleic acids and polypeptides according to the invention are disclosed herein.

NOV1

NOV1 includes three novel transmembrane receptor UNC5H2-like proteins disclosed below. The disclosed sequences have been named NOV1a and NOV1b.

NOV1a

A disclosed NOV1a nucleic acid of 2860 nucleotides (also referred to as GMba58o1_A_da1) encoding a transmembrane receptor UNC5H2-like protein is shown in Table 1A. An open reading frame was identified beginning with an ATG initiation codon at nucleotides 59-61 and ending with a TGA codon at nucleotides 2858-2860. A putative untranslated region upstream from the initiation codon and downstream from the termination codon is underlined in Table 1A. The start and stop codons are in bold letters.

TABLE 1A
NOV1a nucleotide sequence.

(SEQ ID NO:1)

AGACTGGGGCCAGGGAGACAGCCCTGGGGGAGAGGCGCCCGAACCAGGCCGCGGGAGCATGGGGGCCCGGAG
CGGAGCTCGGGGCGCGCTGCTGCTGGCACTGCTGCTCTGCTGGGACCCGAGGCTGAGCCAAGCAGGCACTGA
TTCTGGCAGCGAGGTGCTCCCTGACTCCTTCCCGTCAGCGCCAGCAGAGCCGCTGCCCTACTTCCTGCAGGA
GCCACAGGACGCCTACATTGTGAAGAACAAGCCTGTGGAGCTCCGCTGCCGCGCCTTCCCCGCCACACAGAT
CTACTTCAAGTGCAACGGCGAGTGGGTCAGCCAGAACGACCACGTCACACAGGAAGGCCTGGATGAGGCCAC
CGGTCTGCGGGTGCGCGAGGTGCAGATCGAGGTGTCGCGGCAGCAGGTGGAGGAGCTCTTTGGGCTGGAGGA
TTACTGGTGCCAGTGCGTGGCCTGGAGCTCCGCGGGCACCACCAAGAGTCGCCGAGCCTACGTCCGCATCGC
CTACCTGCGCAAGAACTTCGATCAGGAGCCTCTGGGCAAGGAGGTGCCCCTGGACCATGAGGTTCTCCTGCA
GTGCCGCCCGCCGGAGGGGGTGCCTGTGGCCGAGGTGGAATGGCTCAAGAATGAGGATGTCATCGACCCCAC
CCAGGACACCAACTTCCTGCTCACCATCGACCACAACCTCATCATCCGCCAGGCCCGCCTGTCGGACACTGC
CAACTATACCTGCGTGGCCAAGAACATCGTGGCCAAACGCCGGAGCACCACTGCCACCGTCATCGTCTACGT
GAATGGCGGCTGGTCCAGCTGGGCAGAGTGGTCACCCTGCTCCAACCGCTGTGGCCGAGGCTGGCAGAAGCG
CACCCGGACCTGCACCAACCCCGCTCCACTCAACGGAGGGGCCTTCTGCGAGGGCCAGGCATTCCAGAAGAC
CGCCTGCACCACCATCTGCCCAGTCGATGGGGCGTGGACGGAGTGGAGCAAGTGGTCAGCCTGCAGCACTGA
GTGTGCCCACTGGCGTAGCCGCGAGTGCATGGCGCCCCCACCCCAGAACGGAGGCCGTGACTGCAGCGGGAC
GCTGCTCGACTCTAAGAACTGCACAGATGGGCTGTGCATGCAACTGGAGGCCTCAGGGGATGCGGCGCTGTA
TGCGGGGCTCGTGGTGGCCATCTTCGTGGTCGTGGCAATCCTCATGGCGGTGGGGGTGGTGGTGTACCGCCG
CAACTGCCGTGACTTCGACACAGACATCACTGACTCATCTGCTGCCCTGACTGGTGGTTTCCACCCCGTCAA
CTTTAAGACGGCAAGGCCCAGTAACCCGCAGCTCCTACACCCCTCTGTGCCTCCTGACCTGACAGCCAGCGC
CGGCATCTACCGCGGACCCGTGTATGCCCTGCAGGACTCCACCGACAAAATCCCCATGACCAACTCTCCTCT
GCTGGACCCCTTACCCAGCCTTAAGGTCAAGGTCTACAGCTCCAGCACCACGGGCTCTGGGCCAGGCCTGGC
AGATGGGGCTGACCTGCTGGCGGTCTTGCCGCCTGGCACATACCCTAGCGATTTCGCCCGGGACACCCACTT
CCTGCACCTGCGCAGCGCCAGCCTCGGTTCCCAGCAGCTCTTGGGCCTGCCCCGAGACCCAGGGAGCAGCGT
CAGCGGCACCTTTGGCTGCCTGGGTGGGAGGCTCAGCATCCCCGGCACAGGGGTCAGCTTGCTGGTGCCCAA
TGGAGCCATTCCCCAGGGCAAGTTCTACGAGATGTATCTACTCATCAACAAGGCAGAAAGTACCCTGCCGCT
TTCAGAAGGGACCCAGACAGTATTGAGCCCCTCGGTGACCTGTGGACCCACAGGCCTCCTGCTGTGCCGCCC
CGTCATCCTCACCATGCCCCACTGTGCCGAAGTCAGTGCCCGTGACTGGATCTTTCAGCTCAAGACCCAGGC
CCACCAGGGCCACTGGGAGGAGGTGGTGACCCTGGATGAGGAGACCCTGAACACACCCTGCTACTGCCAGCT
GGAGCCCAGGGCCTGTCACATCCTGCTGGACCAGCTGGGCACCTACGTGTTCACGGGCGAGTCCTATTCCCG
CTCAGCAGTCAAGCGGCTCCAGCTGGCCGTCTTCGCCCCCGCCCTCTGCACCTCCCTGGAGTACAGCCTCCG
GGTCTACTGCCTGGAGGACACGCCTGTAGCACTGAAGGAGGTGCTGGAGCTCGAGCGGACTCTGGGCGGATA
CTTGGTGGAGGAGCCGAAACCGCTAATGTTCAAGGACAGTTACCACAACCTGCGCCTCTCCCTCCATGACCT
CCCCCATGCCCATTGGAGGAGCAAGCTGCTGGCCAAATACCAGGAGATCCCCTTCTATCACATTTGGAGTGG
CAGCCAGAAGGCCCTCCACTGCACTTTCACCCTGGAGAGGCACAGCTTGGCCTCCACAGAGCTCACCTGCAA
GATCTGCGTGCGGCAAGTGGAAGGGGAGGGCCAGATATTCCAGCTGCATACCACTCTGGCAGAGACACCTGC
TGGCTCCCTGGACACTCTCTGCTCTGCCCCTGGCAGCACTGTCACCACCCAGCTGGGACCTTATGCCTTCAA
GATCCCACTGTCCATCCGCCAGAAGATATGCAACAGCCTAGATGCCCCCAACTCACGGGGCAATGACTGGCG
GATGTTAGCACAGAAGCTCTCTATGGACCGGTACCTGAATTACTTTGCCACCAAAGCGAGCCCCACGGGTGT
GATCCTGGACCTCTGGGAAGCTCTGCAGCAGGACGATGGGGACCTCAACAGCCTGGCGAGTGCCTTGGAGGA
GATGGGCAAGAGTGAGATGCTGGTGGCTGTGGCCACCGACGGGGACTGCTGA

In a search of public sequence databases, the NOV1a nucleic acid sequence, located on chromsome 10 has 1604 of 1895 bases (84%) identical to a transmembrane receptor UNC5H2 mRNA from Rattus Norvegicus, (GENBANK-ID: RNU87306). Public nucleotide databases include all GenBank databases and the GeneSeq patent database.

In all BLAST alignments herein, the “E-value” or “Expect” value is a numeric indication of the probability that the aligned sequences could have achieved their similarity to the BLAST query sequence by chance alone, within the database that was searched. For example, the probability that the subject (“Sbjct”) retrieved from the NOV1a BLAST analysis, e.g., transmembrane receptor UNC5H2 mRNA from Rattus Norvegicus, matched the Query NOV1a sequence purely by chance is 0.0. The Expect value (E) is a parameter that describes the number of hits one can “expect” to see just by chance when searching a database of a particular size. It decreases exponentially with the Score (S) that is assigned to a match between two sequences. Essentially, the E value describes the random background noise that exists for matches between sequences.

The Expect value is used as a convenient way to create a significance threshold for reporting results. The default value used for blasting is typically set to 0.0001. In BLAST 2.0, the Expect value is also used instead of the P value (probability) to report the significance of matches. For example, an E value of one assigned to a hit can be interpreted as meaning that in a database of the current size one might expect to see one match with a similar score simply by chance. An E value of zero means that one would not expect to see any matches with a similar score simply by chance. See, e.g., http://www.ncbi.nlm.nih.gov/Education/BLASTinfo/. Occasionally, a string of X's or N's will result from a BLAST search. This is a result of automatic filtering of the query for low-complexity sequence that is performed to prevent artifactual hits. The filter substitutes any low-complexity sequence that it finds with the letter “N” in nucleotide sequence (e.g., “NNNNNNNNNNNNN”) or the letter “X” in protein sequences (e.g., “XXXXXXXXX”). Low-complexity regions can result in high scores that reflect compositional bias rather than significant position-by-position alignment. (Wootton and Federhen, Methods Enzymol 266:554-571, 1996).

The disclosed NOV1a polypeptide (SEQ ID NO: 2) encoded by SEQ ID NO: 1 has 933 amino acid residues and is presented in Table 1B using the one-letter amino acid code. Signal P, Psort and/or Hydropathy results predict that NOV1a has a signal peptide at the first 26 amino acids and is likely to be localized at the plasma membrane with a certainty of 0.5140. In other embodiments, NOV1a is likely to be localized to the microbody (peroxisome) with a certainty of 0.1064, to the endoplasmic reticulum (membrane) with a certainty of 0.1000, or to the endoplasmic reticulum (lumen) with a certainty of 0.1000. The most likely cleavage site for NOV1a is between positions 26 and 27: SQA-GT

TABLE 1B
Encoded NOV1a protein sequence.

(SEQ ID NO:2)

MGARSGARGALLLALLLCWDPRLSQAGTDSGSEVLPDSFPSAPAEPLPYFLQEPQDAYIVKNKPVELRCRAF
PATQIYFKCNGEWVSQNDHVTQEGLDEATGLRVREVQIEVSRQQVEELFGLEDYWCQCVAWSSAGTTKSRRA
YVRIAYLRKNFDQEPLGKEVPLDHEVLLQCRPPEGVPVAEVEWLKNEDVIDPTQDTNFLLTIDHNLIIRQAR
LSDTANYTCVAKNIVAKRRSTTATVIVYVNGGWSSWAEWSPCSNRCGRGWQKRTRTCTNPAPLNGGAFCEGQ
AFQKTACTTICPVDGAWTEWSKWSACSTECAHWRSRECMAPPPQNGGRDCSGTLLDSKNCTDGLCMQLEASG
DAALYAGLVVAIFVVVAILMAVGVVVYRRNCRDFDTDITDSSAALTGGFHPVNFKTARPSNPQLLHPSVPPD
LTASAGIYRGPVYALQDSTDKIPMTNSPLLDPLPSLKVKVYSSSTTGSGPGLADGADLLGVLPPGTYPSDFA
RDTHFLHLRSASLGSQQLLGLPRDPGSSVSGTFGCLGGRLSIPGTGVSLLVPNGAIPQGKFYEMYLLINKAE
STLPLSEGTQTVLSPSVTCGPTGLLLCRPVILTMPHCAEVSARDWIFQLKTQAHQGHWEEVVTLDEETLNTP
CYCQLEPRACHILLDQLGTYVFTGESYSRSAVKRLQLAVFAPALCTSLEYSLRVYCLEDTPVALKEVLELER
TLGGYLVEEPKPLMFKDSYHNLRLSLHDLPHAHWRSKLLAKYQEIPFYHIWSGSQKALHCTFTLERHSLAST
ELTCKICVRQVEGEGQIFQLHTTLAETPAGSLDTLCSAPGSTVTTQLGPYAFKIPLSIRQKICNSLDAPNSR
GNDWRMLAQKLSMDRYLNYFATKASPTGVILDLWEALQQDDGDLNSLASALEEMGKSEMLVAVATDGDC

A search of sequence databases reveals that the NOV1a amino acid sequence has 862 of 945 amino acid residues (91%) identical to, and 897 of 945 amino acid residues (94%) similar to, the 945 amino acid residue 6330415E02RIK protein from Mus musculus (Q9D398) (E=0.0). Public amino acid databases include the GenBank databases, SwissProt, PDB and PIR.

NOV1a is at least expressed in endothelial cells, heart, kidney, adipose, brain (hippocampus), brain (thalamus), cerebral cortex, and the following cancer cell lines: breast cancer, CNS cancer, colon cancer, gastric cancer, lung cancer, melanoma, ovarian cancer and pancreatic cancer at a measurably higher level than the following tissues: adrenal gland, bladder, bone barrow, brain (amygdala), brain (cerebellum), brain (whole), breast, colorectal, liver, lung, lymph nod, mammary gland, ovary, pancreas, pituitary gland, placenta, prostate, salivary gland, skeletal muscle, small intestine, spinal cord, spleen, stomach, testis, thymus, thyroid gland, trachea, and uterus.

NOV1b

A disclosed NOV1b nucleic acid of 2860 nucleotides (also referred to as CG50126-02) encoding a novel beta-adrenergic receptor kinase-like protein is shown in Table 1C. An open reading frame was identified beginning with an ATG codon at nucleotides 59-61, and ending with a TGA codon at nucleotides 2858-2860. Putative untranslated regions, if any, are located upstream from the initiation codon and downstream from the termination codon.

TABLE 1C
NOV1b nucleotide sequence.

(SEQ ID NO:3)

AGACTGGGGCCAGGGAGACAGCCCTGGGGGAGAGGCGCCCGAACCAGGCCGCGGGAACATGGGGGCCCGGAGCGGAGCTC
GGGGCGCGCTGCTGCTGGCACTGCTGCTCTGCTGGGACCCGAGGCTGAGCCAAGCAGGCACTGATTCTGGCAGCGAGGTG
CTCCCTGACTCCTTCCCGTCAGCGCCAGCAGAGCCGCTGCCCTACTTCCTGCAGGAGCCACAGGACGCCTACATTGTGAA
GAACAAGCCTGTGGAGCTTCGCTGCCGCGCCTTCCCCGCCACACAGATCTACTTCAAGTGCAACGGCGAGTGGGTCAGCC
AGAACGACCACGTCACACAGGAAGGCCTGGATGAGGCCACCGGCCTGCGGGTGCGCGAGGTGCAGATCGAGGTGTCGCGG
CAGCAGGTGGAGGAGCTCTTTGGGCTGGAGGATTACTGGTGCCAGTGCGTGGCCTGGAGCTCCGCAGGCACCACCAAGAG
TCGCCGAGCCTACGTCCGCATCGCCTACCTGCGCAAGAACTTCGATCAGGAGCCTCTGGGCAAGGAGGTGCCCCTGGACC
ATGAGGTTCTCCTGCAGTGCCGCCCGCCGGAGGGGGTGCCTGTGGCCGAGGTGGAATGGCTCAAGAATGAGGATGTCATC
GACCCCACCCAGGACACCAACTTCCTGCTCACCATCGACCACAACCTCATCATCCGCCAGGCCCGCCTGTCGGACACTGC
CAACTATACCTGCGTGGCCAAGAACATCGTGGCCAAACGCCGGAGCACCACTGCCACCGTCATCGTCTACGTGAATGGCG
GCTGGTCCAGCTGGGCAGAGTGGTCACCCTGCTCCAACCGCTGTGGCCGAGGCTGGCAGAAGCGCACCCGGACCTGCACC
AACCCCGCTCCACTCAACGGAGGGGCCTTCTGCGAGGGCCAGGCATTCCAGAAGACCGCCTGCACCACCATCTGCCCAGT
CGATGGGGCGTGGACGGAGTGGAGCAAGTGGTCAGCCTGCAGCACTGAGTGTGCCCACTGGCGTAGCCGCGAGTGCATGG
CGCCCCCACCCCAGAACGGAGGCCGTGACTGCAGCGGGACGCTGCTCGACTCTAAGAACTGCACAGATGGGCTGTGCATG
CAACTGGAGGCCTCAGGGGATGCGGCGCTGTATGCGGGGCTCGTGGTGGCCATCTTCGTGGTCGTGGCAATCCTCATGGC
GGTGGGGGTGGTGGTGTACCGCCGCAACTGCCGTGACTTCGACACAGACATCACTGACTCATCTGCTGCCCTGACTGGTG
GTTTCCACCCCGTCAACTTTAAGACGGCAAGGCCCAGTAACCCGCAGCTCCTACACCCCTCTGTGCCTCCTGACCTGACA
GCCAGCGCCGGCATCTACCGCGGACCCGTGTATGCCCTGCAGGACTCCACCGACAAAATCCCCATGACCAACTCTCCTCT
GCTGGACCCCTTACCCAGCCTTAAGGTCAAGGTCTACAGCTCCAGCACCACGGGCTCTGGGCCAGGCCTGGCAGATGGGG
CTGACCTGCTGGGGGTCTTGCCGCCTGGCACATACCCTAGCGATTTCGCCCGGGACACCCACTTCCTGCACCTGCGCAGC
GCCAGCCTCGGTTCCCAGCAGCTCTTGGGCCTGCCCCGAGACCCAGGGAGCAGCGTCAGCGGCACCTTTGGCTGCCTGGG
TGGGAGGCTCAGCATCCCCGGCACAGGGGTCAGCTTGCTGGTGCCCAATGGAGCCATTCCCCAGGGCAAGTTCTACGAGA
TGTATCTACTCATCAACAAGGCAGAAAGTACCCTGCCGCTTTCAGAAGGGACCCAGACAGTATTGAGCCCCTCGGTGACC
TGTGGACCCACAGGCCTCCTGCTGTGCCGCCCCGTCATCCTCACCATGCCCCACTGTGCCGAAGTCAGTGCCCGTGACTG
GATCTTTCAGCTCAAGACCCAGGCCCACCAGGGCCACTGGGAGGAGGTGGTGACCCTGGATGAGGAGACCCTGAACACAC
CCTGCTACTGCCAGCTGGAGCCCAGGGCCTGTCACATCCTGCTGGACCAGCTGGGCACCTACGTGTTCACGGGCCAGTCC
TATTCCCGCTCAGCAGTCAAGCGGCTCCAGCTGGCCGTCTTCGCCCCCGCCCTCTGCACCTCCCTGGAGTACAGCCTCCG
GGTCTACTGCCTGGAGGACACGCCTGTAGCACTGAAGGAGGTGCTGGAGCTGGAGCGGACTCTGGGCGGATACTTGGTGG
AGGAGCCGAAACCGCTAATGTTCAAGGACAGTTACCACAACCTGCGCCTCTCCCTCCATGACCTCCCCCATGCCCATTGG
AGGAGCAAGCTGCTGGCCAAATACCAGGAGATCCCCTTCTATCACATTTGGAGTGGCAGCCAGAAGGCCCTCCACTGCAC
TTTCACCCTGGAGAGGCACAGCTTGGCCTCCACAGAGCTCACCTGCAAGATCTGCGTGCGGCAAGTGGAAGGGGAGGGCC
AGATATTCCAGCTGCATACCACTCTGGCAGAGACACCTGCTGGCTCCCTGGACACTCTCTGCTCTGCCCCTGGCAGCACT
GTCACCACCCAGCTGGGACCTTATGCCTTCAAGATCCCACTGTCCATCCGCCAGAAGATATGCAACAGCCTAGATGCCCC
CAACTCACGGGGCAATGACTGGCGGATGTTAGCACAGAAGCTCTCTATGGACCGGTACCTGAATTACTTTGCCACCAAAG
CGAGCCCCACGGGTGTGATCCTGGACCTCTGGGAAGCTCTGCAGCAGGACGATGGGGACCTCAACAGCCTGGCGAGTGCC
TTGGAGGAGATGGGCAAGAGTGAGATGCTGGTGGCTGTGGCCACCGACGGGGACTGCTGA

In a search of public sequence databases, the NOV1b nucleic acid sequence, located on chromsome 10 has 1604 of 1895 bases (84%) identical to a gb:GENBANK-ID:RNU87306|acc:U87306.1 mRNA from Rattus norvegicus (Rattus norvegicus transmembrane receptor Unc5H2 mRNA, complete cds). (E=0.0) Public nucleotide databases include all GenBank databases and the GeneSeq patent database.

The disclosed NOV1b polypeptide (SEQ ID NO: 4) encoded by SEQ ID NO: 3 has 933 amino acid residues and is presented in Table 1D using the one-letter amino acid code. Signal P. Psort and/or Hydropathy results predict that NOV1b has a signal peptide at the first 26 amino acids and is likely to be localized at the plasma membrane with a certainty of 0.5140. In other embodiments, NOV1b is likely to be localized to the microbody (peroxisome) with a certainty of 0.1064, to the endoplasmic reticulum (membrane) with a certainty of 0.1000, or to the endoplasmic reticulum (lumen) with a certainty of 0.1000. The most likely cleavage site for NOV1b is between positions 26 and 27: SQA-GT

TABLE 1D
Encoded NOV1b protein sequence.

(SEQ ID NO:4)

MGARSGARGALLLALLLCWDPRLSQAGTDSGSEVLPDSFPSAPAEPLPYFLQEPQDAYIVKNKPVELRCRAFPATQIYFK
CNGEWVSQNDHVTQEGLDEATGLRVREVQIEVSRQQVEELFGLEDYWCQCVAWSSAGTTKSRRAYVRIAYLRKNFDQEPL
GKEVPLDHEVLLQCRPPEGVPVAEVEWLKNEDVIDPTQDTNFLLTIDHNLIIRQARLSDTANYTCVAKNIVAKRRSTTAT
VIVYVNGGWSSWAEWSPCSNRCGRGWQKRTRTCTNPAPLNGGAFCEGQAFQKTACTTICPVDGAWTEWSKWSACSTECAH
WRSRECMAPPPQNGGRDCSGTLLDSKNCTDGLCMQLEASGDAALYAGLVVAIFVVVAILMAVGVVVYRRNCRDFDTDITD
SSAALTGGFHPVNFKTARPSNPQLLHPSVPPDLTASAGIYRGPVYALQDSTDKIPMTNSPLLDPLPSLKVKVYSSSTTGS
GPGLADGADLLGVLPPGTYPSDFARDTHFLHLRSASLGSQQLLGLPRDPGSSVSGTFGCLGGRLSIPGTGVSLLVPNGAI
PQGKFYEMYLLINKAESTLPLSEGTQTVLSPSVTCGPTGLLLCRPVILTMPHCAEVSARDWIFQLKTQAHQGHWEEVVTL
DEETLNTPCYCQLEPRACHILLDQLGTYVFTGESYSRSAVKRLQLAVFAPALCTSLEYSLRVYCLEDTPVALKEVLELER
TLGGYLVEEPKPLMFKDSYHNLRLSLHDLPHAHWRSKLLAKYQEIPFYHIWSGSQKALHCTFTLERHSLASTELTCKICV
RQVEGEGQIFQLHTTLAETPAGSLDTLCSAPGSTVTTQLGPYAFKIPLSIRQKICNSLDAPNSRGNDWRMLAQKLSMDRY
LNYFATKASPTGVILDLWEALQQDDGDLNSLASALEEMGKSEMLVAVATDGDC

A search of sequence databases reveals that the NOV1b amino acid sequence has 862 of 945 amino acid residues (91%) identical to, and 893 of 945 amino acid residues (94%) similar to, the 945 amino acid residue ptnr:SPTREMBL-ACC:008722 protein from Rattus norvegicus (Rat) (Transmembrane Receptor UNC5H2) (E=0.0). Public amino acid databases include the GenBank databases, SwissProt, PDB and PIR.

NOV1b is expressed in at least adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea, uterus. This information was derived by determining the tissue sources of the sequences that were included in the invention including but not limited to SeqCalling sources, Public EST sources, and/or RACE sources.

The disclosed NOV1a polypeptide has homology to the amino acid sequences shown in the BLASTP data listed in Table 1E.

TABLE 1E
BLAST results for NOV1a

| Gene Index/ | | Length | Identity | Positives | |
|---|---|---|---|---|---|
| Identifier | Protein/Organism | (aa) | (%) | (%) | Expect |
| ptnr: SPTREMBL- | 6330415E02RIK | 945 | 862/945 | 897/945 | 0.0 |
| ACC: Q9D398 | PROTEIN - Mus | | (91%) | (94%) | |
| | musculus (Mouse) | | | | |
| ptnr: SPTREMBL- | TRANSMEMBRANE | 945 | 862/945 | 893/945 | 0.0 |
| ACC: O08722 | RECEPTOR UNC5H2 | | (91%) | (94%) | |
| ptnr: SPTREMBL- | UNC-5 HOMOLOG (C. | 931 | 610/929 | 723/929 | 0.0 |
| ACC: O08747 | ELEGANS) | | (65%) | (77%) | |
| ptnr: SPTREMBL- | TRANSMEMBRANE | 931 | 598/929 | 718/929 | 0.0 |
| ACC: O95185 | RECEPTOR UNC5C - | | (64%) | (77%) | |
| | Homo sapiens | | | | |

The homology between these and other sequences is shown graphically in the ClustalW analysis shown in Table 1F. In the ClustalW alignment of the NOV1 proteins, as well as all other ClustalW analyses herein, the black outlined amino acid residues indicate regions of conserved sequence (i.e., regions that may be required to preserve structural or functional properties), whereas non-highlighted amino acid residues are less conserved and can potentially be altered to a much broader extent without altering protein structure or function.

[image]

[image]

The presence of identifiable domains in NOV1, as well as all other NOVX proteins, was determined by searches using software algorithms such as PROSITE, DOMAIN, Blocks, Pfam, ProDomain, and Prints, and then determining the Interpro number by crossing the domain match (or numbers) using the Interpro website (http:www.ebi.ac.uk/interpro). DOMAIN results for NOV1as disclosed in Tables 1G-1O, were collected from the Conserved Domain Database (CDD) with Reverse Position Specific BLAST analyses. This BLAST analysis software samples domains found in the Smart and Pfam collections. For Tables 1G-1O and all successive DOMAIN sequence alignments, fully conserved single residues are indicated by black shading or by the sign (|) and “strong” semi-conserved residues are indicated by grey shading or by the sign (+). The “strong” group of conserved amino acid residues may be any one of the following groups of amino acids: STA, NEQK, NHQK, NDEQ, QHRK, MILV, MILF, HY, FYW.

Tables 1G-1O list the domain descriptions from DOMAIN analysis results against NOV1a. This indicates that the NOV1a sequence has properties similar to those of other proteins known to contain this domain.

TABLE 1G
Domain Analysis of NOV1a

gnl|Smart|smarto00218, ZU5, Domain present in ZO-1 and Unc5-like netrin
receptors; Domain of unknown function. (SEQ ID NO:85)
CD-Length = 104 residues, 100.0% aligned
Score = 149 bits (376), Expect = 7e−37

| | | | |
|---|---|---|---|
| Query: | 529 | PGSSVSGTFGCLGGRLSIPGTGVSLLVPNGAIPQGKFYEMYLLINKAESTLPLSEGTQTV | 588 |
| | | \| \|\|\|\|\| \|\|\|\| \| \|\|\| \|++\| \|\|\|\|\|\| \| \|\|+++ \|\| \| \| +\|+ | |
| Sbjct: | 1 | PSFLVSGTFDARGGRLRGPRTGVRLIIPPGAIPQGTRYTCYLVVHDKLSTPPPLEEGETL | 60 |
| Query: | 589 | LSPSVTCGPTGLLLCRPVILTMPHCAEVSARDWIFQLKTQAHQG 632 | |
| | | \|\|\| \| \|\|\| \| \| \|\|\|\|\| +\|\|\|\| + \|\|\| \| + \| | |
| Sbjct: | 61 | LSPVVECGPHGALFLRPVILEVPHCASLRPRDWEIVLLRSENGG 104 | |

TABLE 1H
Domain Analysis of NOV1a

gnl|Pfam|pfam00791, ZU5, ZU5 domain. Domain present in ZO-1 and Unc5-like
netrin receptors Domain of unknown function. (SEQ ID NO:86)
CD-Length = 104 residues, 100.0% aligned
Score = 147 bits (371), Expect = 3e−36

| | | | |
|---|---|---|---|
| Query: | 529 | PGSSVSGTFGCLGGRLSIPGTGVSLLVPNGAIPQGKFYEMYLLINKAESTLPLSEGTQTV | 588 |
| | | \| \|\|\|\|\| \|\|\|\| \| \|\|\| \|++\| \|\|\|\|\|\| \| \|\|+++ \|\| \| \| +\|+ | |
| Sbjct: | 1 | SGFLVSGTFDARGGRLRGPRTGVRLIIPPGAIPQGTRYTCYLVVHDKLSTPPPLEEGETL | 60 |
| Query: | 589 | LSPSVTCGPTGLLLCRPVILTMPHCAEVSARDWIFQLKTQAHQG 632 | |
| | | \|\|\| \| \|\|\| \| \| \|\|\|\|\| +\|\|\|\| + \|\|\| \| + \| | |
| Sbjct: | 61 | LSPVVECGPHGALFLRPVILEVPHCASLRPRDWELVLLRSENGG 104 | |

TABLE 1I
Domain Analysis of NOV1a

gnl|Smart|smart00005, DEATH, DEATH domain, found in proteins involved
in cell death (apoptosis).; Alpha-helical domain present in a variety
of proteins with apoptotic functions. Some (but not all) of these
domains form homotypic and heterotypic dimers. (SEQ ID NO:87)
CD-Length = 96 residues, 99.0% aligned
Score = 64.7 bits (156), Expect = 2e−11

| | | | |
|---|---|---|---|
| Query: | 840 | GPYAFKIPLSIRQKICNSLDAPNSRGNDWRMLAQKLSM-DRYLNYFATKAS-----PTGV | 893 |
| | | \| \| + \|+\|+ \|\| + \|+\|\|\| \|\|+\|\| + + ++ \|++ + | |
| Sbjct: | 1 | PPGAASLTELTREKLAKLLD--HDLGDDWRELARKLGLSEADIDQIETESPRDLAEQSYQ | 58 |
| Query: | 894 | ILDLWEALQQDDGDLNSLASALEEMGKSEMLVAVATD 930 | |
| | | +\| \|\|\| + + \| +\| \|\| +\|\|+ + + + ++ | |
| Sbjct: | 59 | LLRLWEQREGKNATLGTLLEALRKMGRDDAVELLRSE 95 | |

TABLE 1J
Domain Analysis of NOV1a

gnl|Smart|smart00209, TSP1, Thrombospondin type 1 repeats; Type 1
repeats in thrombospondin-1 bind and activate TGF-beta.
(SEQ ID NO:88)
CD-Length = 51 residues, 100.0% aligned
Score = 62.4 bits (150), Expect = 1e−10

| | | | |
|---|---|---|---|
| Query: | 249 | WSSWAEWSPCSNRCGRGWQKRTRTCTNPAPLNGGAFCEGQAFQKTACTT-ICP | 300 |
| | | \| \|+\|\|\|\|\|\| \|\| \| \| \|\|\| \| \| \|\|\| \| \| + \|\| \|\| | |
| Sbjct: | 1 | WGEWSEWSPCSVTCGGGVQTRTRCCNPPP--NGGGPCTGPDTETRACNEQPCP | 51 |

TABLE 1K
Domain Analysis of NOV1a

gnl|Smart|smart00209, TSP1, Thrombospondin type 1 repeats; Type 1
repeats in thrombospondin-1 bind and activate TGF-beta.
(SEQ ID NO:88)
CD-Length = 51 residues, 98.0% aligned
Score = 49.3 bits (116), Expect = 1e−06

| | | | |
|---|---|---|---|
| Query: | 305 | WTEWSKWSACSTECAH-WRSRECMAPPPQNGGRDCSGTLLDSKNCTDGLC | 353 |
| | | \| \|\|\|+\|\| \|\| \| ++\| \|\| \|\|\| \|+\| +++ \| + \| | |
| Sbjct: | 1 | WGEWSEWSPCSVTCGGGVQTRTRCCNPPPNGGGPCTGPDTETRACNEQPC | 50 |

TABLE 1L
Domain Analysis of NOV1a

gnl|Pfam|pfam00531, death, Death domain. (SEQ ID NO:89)
CD-Length = 83 residues, 98.8% aligned
Score = 57.4 bits (137), Expect = 4e−09

| | | | |
|---|---|---|---|
| Query: | 852 | QKICNSLDAPNSRGNDWRMLAQKLSM-DRYLNYFATKA----SPTGVILDLWEALQQDDG | 906 |
| | | +++\| \|\| \| \| \|\|\| \|\|+\|\| + + ++ + \|\|\| +\|\|\|\|\| + | |
| Sbjct: | 1 | RELCKLLDDP--LGRDWRRLARKLGLSEEEIDQIEHENPRLASPTYQLLDLWEQRGGKNA | 58 |
| Query: | 907 | DLNSLASALEEMGKSEMLVAVATD 930 | |
| | | + +\| \|\| +\|\|+ + + + + | |
| Sbjct: | 59 | TVGTLLEALRKMGRDDAVELLESA 82 | |

TABLE 1M
Domain Analysis of NOV1a

gn1|Pfam|pfam00090, tsp_1, Thrombospondin type 1 domain.
(SEQ ID NO:90)
CD-Length = 48 residues, 91.7% aligned
Score = 49.7 bits (117), Expect = 7e−07

| | | | |
|---|---|---|---|
| Query: | 250 | SSWAEWSPCSNRCGRGWQKRTRTCTNPAPLNGGAFCEGQAFQKTACT | 296 |
| | | \| \|+\|\|\|\|\|\| \|\|+\| \| \| \|\|\| +\|\| \|\| \| \| \| + \|\| | |
| Sbjct: | 1 | SPWSEWSPCSVTCGKGIRTRQRTCNSPA---GGKPCTGDAQETEACM | 44 |

TABLE 1N
Domain Analysis of NOV1a

gn1|Smart|smart00409, IG, Immunoglobulin (SEQ ID NO:91)
CD-Length = 86 residues, 100.0% aligned
Score = 48.9 bits (115), Expect = 1e−06

| | | | |
|---|---|---|---|
| Query: | 159 | PLGKEVPLDHEVLLQCRPPEGVPVAEVEWLKNEDVIDPTQDTNFLLTIDHN---LIIRQA | 215 |
| | | \| \| \| \| \| \| \| \| \| \| + + \| ++ \| \| | |
| Sbjct: | 1 | PPSVTVKEGESVTLSCEAS-GNPPPTVTWYKQGGKL-LAESGRFSVSRSGGNSTLTISNV | 58 |
| Query: | 216 | RLSDTANYTCVAKNIVAKRRSTTATVIVY 244 | |
| | | \|+ \|\|\| \| \| \| \| \|+ \| | |
| Sbjct: | 59 | TPEDSGTYTCAATNSSGSASSGT-TLTVL 86 | |

TABLE 10
Domain Analysis of NOV1a

gn1|Smart|smart00408, IGc2, Immunoglobulin C-2 Type (SEQ ID NO:92)
CD-Length = 63 residues. 87.3% aligned
Score = 42.7 bits (99), Expect = 9e−05

| | | | |
|---|---|---|---|
| Query: | 170 | VLLQCRPPEGVPVAEVEWLKNEDVIDPTQDTNFLLTIDHNLIIRQARLSDTANYTCVAKN | 229 |
| | | \| \| \| \| \| \|\| + \|\|\|+ + ++ ++ \| \|+ \| \|+ \|\|\|\|\|+\| | |
| Sbjct: | 6 | VTLTC-PASGDPVPNITWLKDGKPLPESR----VVASGSTLTIKNVSLEDSGLYTCVARN | 60 |

Migration of neurons from proliferative zones to their functional sites is fundamental to the normal development of the central nervous system. Disruption of the mouse rostral cerebellar malformation mutation (rcm) gene, also called the Unc5h3 gene, resulted in a failure of tangentially migrating granule cells to recognize the rostral boundary of the cerebellum. In rcm-mutant mice, the cerebellum is smaller and has fewer folia than in wildtype, ectopic cerebellar cells are present in midbrain regions by 3 days after birth, and there are abnormalities in postnatal cerebellar-neuronal migration. Ackerman et al. (1997). Sequence analysis has revealed that the predicted rcm mouse protein is a transmembrane protein that contains 2 immunoglobulin (Ig)-like domains and 2 type I thrombospondin (THBS1) motifs in the extracellular region. Ig and THBS1 domains are also found in the extracellular region of the C. elegans UNC5 transmembrane protein, and the C-terminal 865-amino acid region of Rcm is 30% identical to UNC5. In addition, the UNC5 protein is essential for dorsal guidance of pioneer axons and for the movement of cells away from the netrin ligand. Ackerman et al. (1997). In the developing brain of vertebrates, netrin-1 plays a role in both cell migration and axonal guidance.

In the developing nervous system, migrating cells and axons are guided to their targets by cues in the extracellular environment. The netrins are a family of phylogenetically conserved guidance cues that can function as diffusible attractants and repellents for different classes of cells and axons. In vertebrates, insects and nematodes, members of the DCC subfamily of the immunoglobulin superfamily have been implicated as receptors that are involved in migration towards netrin sources. In Caenorhabditis elegans, the transmembrane protein UNC-5 has been implicated in these responses, as loss of UNC-5 function causes migration defects and ectopic expression of UNC-5 in some neurons can redirect their axons away from a netrin source.

The disclosed NOV1 nucleic acid of the invention encoding a UNC5H2-like protein includes the nucleic acid whose sequence is provided in Table 1A, 1C or a fragment thereof. The invention also includes a mutant or variant nucleic acid any of whose bases may be changed from the corresponding base shown in Table 1A or 1C while still encoding a protein that maintains its UNC5H2 like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 16 percent of the bases may be so changed.

The disclosed NOV1 protein of the invention includes the UNC5H2-like protein whose sequence is provided in Table 1B or 1D. The invention also includes a mutant or variant protein any of whose residues may be changed from the corresponding residue shown in Table 1B or 1D while still encoding a protein that maintains its UNC5H2-like activities and physiological functions, or a functional fragment thereof. In the mutant or variant protein, up to about 9 percent of the residues may be so changed.

The invention further encompasses antibodies and antibody fragments, such as F_{ab} or (F_{ab})₂, that bind immunospecifically to any of the proteins of the invention.

The above defined information for this invention suggests that this UNC5H2-like protein (NOV1) may function as a member of a “UNC5H2 family”. Therefore, the NOV1 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV 1 nucleic acids and proteins of the invention are useful in potential therapeutic applications implicated in cancer including but not limited to various pathologies and disorders as indicated below. For example, a cDNA encoding the UNC5H2-like protein (NOV1) may be useful in gene therapy, and the UNC5H2 -like protein (NOV1) may be useful when administered to a subject in need thereof. By way of nonlimiting example, the compositions of the present invention will have efficacy for treatment of patients suffering from cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, obesity, transplantation, diabetes, autoimmune disease, renal artery stenosis, interstitial nephritis, glomerulonephritis, polycystic kidney disease, systemic lupus erythematosus, renal tubular acidosis, IgA nephropathy, hypercalceimia, Lesch-Nyhan syndrome, Von Hippel-Lindau (VHL) syndrome, Alzheimer's disease, stroke, tuberous sclerosis, Parkinson's disease, Huntington's disease, cerebral palsy, epilepsy, Lesch-Nyhan syndrome, multiple sclerosis, ataxia-telangiectasia, leukodystrophies, behavioral disorders, addiction, anxiety, pain, neuroprotection, cancers, and/or other pathologies and disorders. For example, a cDNA encoding the transmembrane receptor UNC5H2-like protein may be useful in transmembrane receptor UNC5H2 therapy, and the transmembrane receptor UNC5H2-like protein may be useful when administered to a subject in need thereof. By way of nonlimiting example, the compositions of the present invention will have efficacy for treatment of patients suffering from cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, obesity, transplantation, diabetes, autoimmune disease, renal artery stenosis, interstitial nephritis, glomerulonephritis, polycystic kidney disease, systemic lupus erythematosus, renal tubular acidosis, IgA nephropathy, hypercalceimia, Lesch-Nyhan syndrome, Von Hippel-Lindau (VHL) syndrome, Alzheimer's disease, stroke, tuberous sclerosis, Parkinson's disease, Huntington's disease, cerebral palsy, epilepsy, Lesch-Nyhan syndrome, multiple sclerosis, ataxia-telangiectasia, leukodystrophies, behavioral disorders, addiction, anxiety, pain, neuroprotection, cancers, and other diseases, disorders and conditions of the like. Also since this gene is expressed at a measurably higher level in several cancer cell lines (including breast cancer, CNS cancer, colon cancer, gastric cancer, lung cancer, melanoma, ovarian cancer and pancreatic cancer), it may be useful in diagnosis and treatment of these cancers. The NOV1 nucleic acid encoding the UNC5H2-like protein of the invention, or fragments thereof, may further be useful in diagnostic applications, wherein the presence or amount of the nucleic acid or the protein are to be assessed.

NOV1 nucleic acids and polypeptides are further useful in the generation of antibodies that bind immuno-specifically to the novel NOV1 substances for use in therapeutic or diagnostic methods. These antibodies may be generated according to methods known in the art, using prediction from hydrophobicity charts, as described in the “Anti-NOVX Antibodies” section below. The disclosed NOV1 proteins have multiple hydrophilic regions, each of which can be used as an immunogen. In one embodiment, a contemplated NOV1 epitope is from about amino acids 1 to 100. In another embodiment, a NOV1 epitope is from about amino acids 200 to 300. In further embodiments, a NOV1 epitope is from about amino acids 450 to 500, from about amino acids 600 to 900, from about amino acids 950 to 1000, from about amino acids 1200 to 1300, from about amino acids 1400 to 1600, from about amino acids 1800 to 1900, from about amino acids 1950 to 2050, and from about amino acids 2200 to 2300. These novel proteins can be used in assay systems for functional analysis of various human disorders, which will help in understanding of pathology of the disease and development of new drug targets for various disorders.

NOV2

NOV2 includes three novel protein tyrosine phosphatase precursor-like proteins disclosed below. The disclosed sequences have been named NOV2a, NOV2b, and NOV2c.

NOV2a

A disclosed NOV2a nucleic acid of 6994 nucleotides (also referred to as SC126422078_A) encoding a receptor protein tyrosine phosphatase precursor-like protein is shown in Table 2A. An open reading frame was identified beginning with an ATG initiation codon at nucleotides 31-33 and ending with a TAA codon at nucleotides 6874-6876. A putative untranslated region upstream from the initiation codon and downstream from the termination codon is underlined in Table 2A. The start and stop codons are in bold letters.

TABLE 2A
NOV2a nucleotide sequence.

(SEQ ID NO:5)

TGATTCTACTGGCTGAAAAATGTAATAAAGATGGATTTTCTTATCATTTTTCTTTTACTTTTTATTGGGACT
TCAGAGACACAGGTAGATGTTTCCAATGTCGTTCCTGGTACTAGGTACGATATAACCATCTCTTCAATTTCT
ACAACATACACCTCACCTGTTACTAGAATAGGGGCTTCTAATGAACCAGGGCCTCCAGTCTTCCTAGCCGGG
GAAAGAGTCGGATCTGCTGGGATTCTTCTGTCTTGGAATACACCACCTAATCCAAATGGAAGGATTATATCT
TACATTGTCAAATATAAGGAAGTTTGTCCGTGGATGCAAACAGTATATACACAAGTCAGATCAAAGCCAGAC
AGTCTGGAAGTTCTTCTTACTAATCTTAATCCTGGAACAACATATGAAATTAAGGTAGCTGCTGAAAACAGT
GCTGGCATTGGAGTGTTTAGTGATCCATTTCTCTTCCAAACTGCAGAAAGTGCTCCAGGAAAAGTGGTGGAT
TTCACAGGTGAGGCTGTCCCGTTCAGCAGTAAGCTGATGTGGTATACCTCGGCAACCAAAAAAAAAATTACC
AGCTTCAAGATTAGTGTCAAGCATAACAGAAGTGGGATAGTAGTGAAAGAACTGTCAATCAGAGTGGAGTGC
ATTTTAAGTGCTTCCCTTCCTTTCCACTGCAACGAGAATAGTGAATCTTTTTTATCGAGTACAGCCAGCCCT
TCTCCAACCCTTGGTAGAGTTACACCTCCATCGCGTACCACACATTCATCAAGCACGTTGACACAGAATGAG
ATCAGCTCTGTGAAAGAGCCTATCAGTTTTGTAGTGACACACTTGAGACCTTATACAACATATCTTTTTGAA
GTTTCAGCTGCTACAACTGAAGCAGGTTATATTGATAGTACCATTGTCAGAACACCAGAATCAGTGCCTGAA
GGACCACCACAAAACTGCGTAACAGGCAACATCACAGGAAAGTCCTTTTCAATTTTATGGGACCCACCAACT
ATAGTAACAGGGAAATTTAGTTATAGAGTTGAATTATATGGACCATCAGCACGTCGCATTTTGGATAACAGC
ACAAAAGACCTCAAGTTTGCATTCACTAACCTAACACCATTTACAATGTATGATGTCTATATTGCGGCTGAA
ACCAGTGCAGGGACTGGGCCCAAGTCAAATATTTCAGTATTCACTCCACCAGATGTTCCAGGGGCAGTGTTT
GATTTACAACTTGCAGACGTAGAATCCACGCAAGTAAGAATTACTTGGAAGAAACCACGACAACCAAATGGA
ATTATThACCAATACCGAGTGAAAGTGCTAGTTCCAGAGACAGGAATAATTTTGGAAAATACTTTGCTCACT
GGAAATAATGAGATAAATGACCCCATGGCTCCAGAAATTGTGAACATAGTACAGCCAATCGTAGGATTATAT
GAGGGTTCAGCAGAGATGTCGTCTGACCPTCACTCACTTGCTACATTTATATATAACAGCCATCCAGATAAA
AACTTTCCTGCAACGAATAGAGCTGAAGACCAGACTTCACCAGTTGTAACTACAAGGAATCAGTATATTACT
GACATTGCAGCTGAACAGCTGACTTATCTTCTTATCAGATTAAGGAGATTTTGGGCTGAGACAATGGGGTTT
TCTAGATATACAATCATGTCATCTGCAAGCAGGGACAATTTGACTTCCCCAGGCCCTTTGTCAGCCCAAAAT
TTCAGAGTTACACATGTTACCATAACAGAAGTATTTTTACACTGGGATCCTCCAGATCCTGTATTTTTTCAT
CATTACCTTATCACTATTTTGGATGTTGAAAACCAATCCAAGAGTATTATTTTAAGGACATTAAACAGTTTG
TCTCTTGTCCTTATAGGGTTAAAGAAATACACAAAATACAAAATGAGAGTGGCAGCCTCAACCCACGTTGGA
GAAAGTTCTTTGTCTGAAGAAAATGACATCTTTGTGAGAACTTCAGAAGATGAACCGGAATCATCACCTCAA
GATGTCGAAGTAATTGATGTTACCGCAGATGAAATAAGGTTGAAGTGGTCACCACCCGAAAAGCCCAATGGC
ATCATTATTGCTTATGAAGTGCTATATAAAAATATAGATACTTTATATATGAAGAACACATCAACAACAGAC
ATAATATTAAGGAACTTAAGACCTCACACCCTCTATAACATTTCTGTAAGGTCTTACACCAGATTTGGTCAT
GGCAATCAGGTATCTTCTTTACTCTCTGTAAGGACTTCGGAGTCAGTGCCTCATAGTGCACCAGAAAATATC
ACTTACAAAAATATTTCTTCTGGACAGATTGAGCTATCATTCCTTCCCCCAAGTAGTCCCAATGGAATCATA
CAAAAATATACAATTTATCTCAAGAGAAGTAATGGAAATGAGGAAAGAACTATAAATACAACCTCTTTAACC
CAAAACATTAAAGGTCTCAAGAAATATACCCAATATATCATTGAGGTGTCTGCTAGTACACTCAAAGGTGAA
GGAGTTCGGAGTGCTCCCATAAGTATACTGACGGAGGAAGATGCTCCTGATTCTCCCCCTCAAGACTTCTCT
GTAAAACAGTTGTCTGGTGTCACGGTGAAGTTGTCATGGCAACCACCCCTGGAGCCAAATGGAATTATCCTT
TATTACACAGTTTATGTCTGGAGATCATCATTAAAAACTATTAATGTCACTGAAACATCATTGGAGTTATCA
GATTTGGATTATAATGTTGAATACAGTGCTTATGTAACAGCTAGCACCAGATTTGGTGATGGGAAAACAAGA
AGCAATATCATTAGCTTTCAAACACCAGAGGGACCAAGCGATCCTCCCAAAGATGTTTATTATGCAAACCTC
AGTTCTTCATCAATAATTCTTTTCTGGACACCTCCTTCAAAACCTAATGGGATTATACAATATTACTCTGTT
TATTACAGAAATACTTCAGGTACTTTTATGCAGAATTTTACACTCCATGAAGTAACCAATGAcTTTGACAAT
ATGACTGTATCCACAATTATAGATAAACTGACAATATTCAGCTACTATACATTTTGGTTAACACCAAGTACT
TCAGTTGGAAATGGGAATAAAAGCAGTGACATCATTGAAGTATACACAGATCAAGACGTACCTGAAGGGTTT
GTTGGAAACCTGACTTACGAATCCATTTCGTCAACTGCAATAAATGTAACCTGGGTCCCACCGGCTCAACCA
AACGGTCTAGTCTTCTACTATGTTTCACTGATCTTACAGCAGACTCCTCGCCATGTGAGACCACCTCTTGTT
ACATATGAGAGAAGCATATATTTTGATAATCTGGAAAAATACACTGATTATATATTAAAAATTACTCCATCA
ACAGAAAACCGATTCTCTGATACCTATACTGCCCAGCTATACATCAAGACTGAAGAAGATATCCCAGAAACT
TCACCAATAATCAACACTTTTAAAAACCTTTCCTCTACCTCAGTTCTCTTATCATGGGATCCCCCAGTAAAG
CCAAATGGTGCAATAATAAGTTATGATTTAACTTTACAAGGACCAAATGAAAATTATTCTTTCATTACTTCT
GATAATTACATAATATTGGAAGAGCTTTCACCATTTACATTATATAGCTTTTTTGCTGCCGCAAGAACTAGA
AAAGGACTTGGTCCTTCCAGTATTCTTTTCTTTTACACAGATGAGTCAGTGCCGTTAGCACCTCCACAAAAT
TTGACTTTAATCAACTGTACTTCAGACTTTGTNTGGCTGAAATGGAGCCCAAGTCCTCTTCCAGGTGGTATT
GTTAAAGTATATAGTTTTAAAATTCATGAACATGAAACTGACACTATATATTATAAOAATATATCAGCATTT
AAAACTGAAGCCAAACTTGTTGGACTGGAACCAGTCAGCACCTACTCTATCCGTGTATCTGCGTTCACCAAA
GTTGCAAATGGCAATCAATTTAGTAATGTAGTAAAATTCACAACCCAAGAATCAGTTCCAGATGTCGTGCAG
AATATGCAGTGCATGGCAACTAGCTGGCAGTCAGTTTTAGTGAAATGGGATCCACCCAAAAAGGCAAATGGA
ATAATAACGCAGTATATGGTAACAGTTGAAACGAATTCTACAAAAGTTTCTCCCCAAGATCACATGTACACT
TTCATAAAGCTTCTTGCCAATACCTCATATGTCTTTAAAGTAAGAGCTTCAACCTCAGCTGGTGAAGGTGAT
GAAAGCACATGCCATGTCAGCACACTACCTGAAACAGTTCCCAGTGTTCCCACAAATATTGCTTTTTCTGAT
GTTCAGTCAACTAGTGCAACATTGACATGGATAAGACCTGACACTATCCTTGGCTACTTTCAAAATTACAAA
ATTACCACTCAACTTCGTGCTCAAAAATGCAAAGAATGGGAATCCGAAGAATGTGTTGAATATCAAAAAATT
CAATACCTCTATGAAGCTCACTTAACTGAAGAGACAGTATATGGATTAAAGAAATTTAGATCGTATAGATTC
CAAGTGGCTGCCAGCACCAATGCTGGCTATGGCAATGCTTCAAACTGGATTTCTACAAAAACTCTGCCTGGC
CCTCCAGATGGTCCTCCTGAAAATGTTCATGTAGTAGCAACATCACCTTTTAGCATCAGCATAAGCTGGAGT
GAACCTGCTGTCATTACTGGACCAACATGTTATCTGATTGATGTCAAATCGGTAGATAATGATGAATTTAAT
ATATCCTTCATCAAGTCAAATGAAGAAAATAAAACCATAGAAATTAAAGATTTAGAAATATTCACAAGGTAT
TCTGTAGTGATCACTGCATTTACTGGGAACATTAGTGCTGCATATGTAGAAGGGAAGTCAAGTGCTGAAATG
ATTGTTACTACTTTAGAATCAGCCCCAAACCACCCACCTAACAACATCACATTTCAGAACATACCAGATGAA
GTTACAAAATTTCAATTAACGTTCCTTCCTCCTTCTCAACCTAATGGAAATATCCAAGTATATCAAGCTCTG
GTTTACCGAGAAGATGATCCTACTGCTGTCCAGATTCACAACCTCAGTATTATACAGAAAACCAACACATTC
GTCATTGCAATGCTACAAGGACTAAAAGGTGGACATACATACAATATCAGTGTTTACGCAGTCAATAGTGCT
GGTGCAGGTCCAAAGGTTCCGATGAGAATAACCATGGATATCAAAGCTCCAGCACGACCAAAAACCAAACCA
ACCCCTATTTATGATGCCACAGGAAAACTGCTTGTGACTTCAACAACAATTACAATCAGAATGCCAATATGT
TACTACAGTGATGATCATGCACCAATAAAAAATGTACAAGTGCTTGTGACAGAAACAGCAGCTCAGCATGAT
GGAAATGTAACAAAGTGGTATGATGCATATTTTAATAAAGCAAGGCCATATTTTACAAATGAAGGCTTTCCT
AACCCTCCATGTACAGAAGGAAAGACAAAGTTTAGTGGCAATGAAGAAATCTACATCATAGGTGCTGATAAT
GCATGCATGATTCCTGGCAATGAAGACAAAATTTGCAATGGACCACTGAAACCAAAAAAGCAATACTTATTT
AAATTTAGAGCTACAAATATTATGGGACAATTTACTGACTCTGATTATTCTGACCCTGTTAAGACTTTAGGC
GAAGGACTTTCAGAAAGAACCGTAGAGATCATTCTTTCCGTCACTTTGTGTATCCTTTCAATAATTCTCCTT
GGAACAGCTATTTTTGCATTTGCAAGAATTCGACAGAAGCAGAAAGAAGGTGGCACATACTCTCCTCAGGAT
GCAGAAATTATTGACACTAAATTGAAGCTGGATCAGCTCATCACAGTGGCAGACCTGGAACTGAAGGACGAG
AGATTAACGCGGCCAATAAGCAAGAAATCCTTCCTGCAACATGTTGAACAGCTTTGCACAAACAACAACCTA
AAGTTTCAAGAAGAATTTTCGGAATTACCAAAATTTCTTCAGGATCTTTCTTCAACTGATCCTGATCTGCCT
TGGAATAGAGCAAAAAACCGCTTCCCAAACATAAAACCATATAATAATAACAGAGTAAAGCTGATAGCTGAC
GCTAGTGTTCCAGGTTCGGATTATATTAATGCCAGCTATATTTCTGGTTATTTATGTCCAAATGAATTTATT
GCTACTCAAGGTCCACTACCAGGAACAGTTGGAGATTTTTGGAGAATGGTGTGGGAAACCAGAGCAAAAACA
TTAGTAATGCTAACACAGTGTTTTGAAAAAGGACGGATCAGATGCCATCAGTATTGGCCAGACCACAACAAG
CCAGTTACTGTCTTTGGAGATATAGTGATTACAAAGCTAATGGAGGATGTTCAAATAGATTGGACTATCAGG
GATCTGAAAATTGAAAGGCATGGGGATTGCATGACTGTTCGACAGTGTAACTTTACTGCCTGGCCAGAGCAT
GGGGTTCCTGAGAACAGCGCCCCTCTAATTCACTTTGTGAAGTTGGTTCGAGCAAGCACGCCACATGACACC
ACACCTATGATTGTTCACTGCAGTGCTGGAGTTGGAAGAACTGGAGTTTTTATTGCTCTGGACCATTTAACA
CAACATATAAATGACCATGATTTTGTGGATATATATGGACTAGTAGCTCAACTGAGAAGTGAAAGAATGTGC
ATGGTGCAGAATCTGGCACAGTATATCTTTTTACACCAGTGCATTCTGGATCTCTTATCAAATAAGGGAAGT
AATCAGCCCATCTGTTTTGTTAACTATTCAGCACTTCAGAAGATGGACTCTTTGGACGCCATGGAAGGTGGT
GATGTTGAGCTTGAATGGGAAGAAACCACTATGTAAATATTCAGACCAAAGGATACAATTGGAAGAGATTTT
TAAATCCCAGGGGCCAAAGTTACCCCCTCATTCTTCCGAATTGAAATGTGCAACCTTAAAGAAATATCTATC
CTTCTCTCAC

In a search of public sequence databases, the NOV2a nucleic acid sequence, located on chromsome 12 has 777 of 3293 bases (84%) identical to a gb:GENBANK-ID:AF063249|acc:AF063249.1 mRNA from Rattus norvegicus (Rattus norvegicus glomerular mesangial cell receptor protein-tyrosine phosphatase precursor (PTPRQ) mRNA, complete cds) (E=0.0). Public nucleotide databases include all GenBank databases and the GeneSeq patent database.

The disclosed NOV2a polypeptide (SEQ ID NO: 6) encoded by SEQ ID NO: 5 has 2281 amino acid residues and is presented in Table 2B using the one-letter amino acid code. Signal P, Psort and/or Hydropathy results predict that NOV2a has a signal peptide and is likely to be localized in the plasma membrane with a certainty of 0.4600. In other embodiments, NOV2a may also be localized to the microbody (peroxisome) with acertainty of 0.1381, the endoplasmic reticulum (membrane) with a certainty of 0.1000 or in the endoplasmic reticulum (lumen) with a certainty of 0.1000. The most likely cleavage site for a NOV2a peptide is between amino acids 17 and 18, at: SET-QV.

TABLE 2B
Encoded NOV2a protein sequence.

(SEQ ID NO:6)

MDFLIIFLLLFIGTSETQVDVSNVVPGTRYDITISSISTTYTSPVTRIGASNEPGPPVFLAGERVGSAGILL
SWNTPPNPNGRIISYIVKYKEVCPWMOTVYTQVRSKPDSLEVLLTNLNPGTTYEIKVAAENSAGIGVFSDPF
LFQTAESAPGKVVDFTGEAVPFSSKLMWYTSATKKKITSFKISVKHNRSGIVVKEVSIRVECILSASLPLHC
NENSESFLWSTASPSPTLGRVTPPSRTTHSSSTLTQNEISSVKEPISFVVTHLRPYTTYLFEVSAATTEAGY
IDSTIVRTPESVPEGPPQNCVTGNITGKSFSILWDPPTIVTGKFSYRVELYGPSAGRILDNSTKDLKFAFTN
LTPFTMYDVYIAAETSAGTGPKSNISVFTPPDVPGAVFDLQLAEVESTQVRITWKKPRQPNGIINQYRVKVL
VPETGIILEWTLLTGNNEINDPMAPETVNIVQPMVCLYEGSAEMSSDLHSLATFIYNSHPDKNFPARNPAED
QTSPVVTTRNQYITDIAAEQLTYVLIRLRRFWAETMGFSRYTIMSSASRDNLTSPGPLSAQNFRVTHVTITE
VFLHflPPDPVFFHHYLITILDVENQSKSIILRTLNSLSLVLIGLKKYTKYKMRVAASTHVGESSLSEENDI
FVRTSEDEPESSPQDVEVIDVTADEIRLKWSPPEKPNGIIIAYEVLYKJIDTLYMKNTSTTDIILRNLRPHT
LYNISVRSYTRFGHGNQVSSLLSVRTSESVPDSAPENITYKNISSGEIELSFLPPSSPNGTIQKYTIYLKRS
NGNEERTINTTSLTQNIKGLKKYTQYIIEVSASTLKGEGVRSAPISILTEEDAPDSPPQDFSVKQLSGVTVK
LSWQPPLEPNGIILYYTVYVWRSSLKTINVTETSLELSDLDYNVEYSAYVTASTRFGDGKTRSNIISFQTPE
GPSDPPKDVYYANLSSSSIILFWTPPSKPNGIIQYYSVYYRNTSGTFMQNFTLHEVTNDFDNMTVSTIIDKL
TIFSYYTFWITASTSVGNGNKSSDIIEVYTDQDVPEGFVGNLTYESISSTAINVSWVPPAQPNGLVFYYVSL
ILQQTPRHVRPPLVTYERSIYFDNLEKYTDYILKITPSTEKGFSDTYTAQLYIKTEEDIPETSPIINTFKNL
SSTSVLLSWDPPVKPNGAIISYDLTLQGPNENYSFITSDNYIILEELSPFTLYSFFAAARTRKGLGPSSILF
FYTDESVPLAPPQNLTLINCTSDFVWLKWSPSPLPGGIVKVYSFKIHEHETDTIYYKNISGFKTEAKLVGLE
PVSTYSIRVSAFTKVGNGNQFSNVVKFTTQESVPDVVQNMQCMATSWQSVLVKWDPPKKANGIITQYMVTVE
RNSTKVSPQDHMYTFIKLLANTSVVFKVRASTSAGEGDESTCHVSTLPETVPSVPTNIAFSDVQSTSATLTW
IRPDTILGYFQNYKITTQLRAQKCKEWESEECVEYQKIQYLYEAHLTEETVYGLKKFRWYRFQVAASTNAGY
GNASNWISTKTLPGPPDGPPENVHVVATSPFSISISWSEPAVITGPTCYLIDVKSVDNDEFNISFIKSNEEN
KTIEIKDLEIFTRYSVVITAFTGNISAAYVEGKSSAEMIVTTLESAPKDPPNNMTFQKIPDEVTKFQLTFLP
PSQPNGNIQVYQALVYREDDPTAVQIHNLSIIQKTNTFVIAMLEGLKGGHTYNISVYAVNSAGAGPKVPMRI
TMDIKAPARPKTKPTPIYDATGKLLVTSTTITIRMPICYYSDDHGPIKNVQVLVTETGAQHDGNVTKWYDAY
FNKARPYFTNEGFPNPPCTEGKTKFSGNEEIYIIGADNACMIPGNEDKICNGPLKPKKQYLFKFRATNIMGQ
PTDSDYSDPVKTLGEGLSERTVEIILSVTLCILSIILLQTAIFAPARIRQKQKEGCTYSPQDAEIIDTKLKL
DQLITVADLELKDERLTRPISKKSFLQUVEELCTNNNLKFQEEFSELPKFLQDLSSTDADLPWNRAKNRFPN
IKPYNNNRVKLIADASVPGSDYINASYISGYLCPNEFIATQGPLPGTVGDFWRMVWETRAKTLVMLTQCFEK
GRIRCHQYWPEDNKPVTVFGDIVITKLMEDVQIDWTIRDLKIERHGDCMTVRQCNFTAWPEHGVPENSAPLI
HFVKLVRASRAHDTTPMIVHCSAGVGRTGVFIALDHLTQHINDHDFVDIYGLVAELRSERMCMVQNLAQYIF
LHQCILDLLSNKGSNQPICFVNYSALQKMDSLDAMEGGDVELEWEETTM

A search of sequence databases reveals that the NOV2a amino acid sequence has 1894 of 2301 amino acid residues (82%) identical to, and 2078 of 2301 amino acid residues (90%) similar to, the 2302 amino acid residue ptnr:SPTREMBL-ACC:088488 protein from Rattus norvegicus (Rat) (Glomerular Mesangial Cell Receptor Protein-Tyrosine Phosphatase Precursor (EC 3.1.3.48)) (E=0.0). Public amino acid databases include the GenBank databases, SwissProt, PDB and PIR.

NOV2 is expressed in at least kidney and colon. This information was derived by determining the tissue sources of the sequences that were included in the invention including but not limited to SeqCalling sources, Public EST sources, Literature sources, and/or RACE sources.

In addition, the sequence is predicted to be expressed in Rattus norvegicus: kidney because of the expression pattern of (GENBANK-ID: gb:GENBANK-ID:AF063249|acc:AF063249.1) a closely related Rattus norvegicus glomerular mesangial cell receptor protein-tyrosine phosphatase precursor (PTPRQ) mRNA, complete cds homolog.

NOV2b

A disclosed NOV2b nucleic acid of 2565 nucleotides (also referred to as CG50718-02) encoding a novel Glomerular Mesangial Cell Receptor Protein-Tyrosine-like protein is shown in Table 2C. An open reading frame was identified beginning with an AGA codon at nucleotides 1-3 and ending with a GAG codon at nucleotides 2563-2565. The start and stop codons are in bold letters in Table 2C. Because the first and last codons are not traditional initiation and termination codons, NOV2b could represent a partial reading frame that extends in the 5′ and/or 3′ directions.

TABLE 2C
NOV2b nucleotide sequence.

(SEQ ID NO:7)

AGATCTCCTGAAGGGTTTGTTGGAAACCTGACTTACGAATCCATTTCGTCAACTGCAATAAATGTAAGCTGG
GTCCCACCGGCTCAACCAAACGGTCTAGTCTTCTACTATGTTTCACTGATCTTACAGCAGACTCCTCGCCAT
GTGAGACCACCTCTTGTTACATATGAGAGAAGCATATATTTTGATAATCTGGAAAAATACACTGATTATATA
TTAAAAATTACTCCATCAACAGAAAAGGGATTCTCTGATACCTATACTGCCCAGCTATACATCAAGACTGAA
GAAGATGTCCCAGAAACTTCACCAATAATCAACACTTTTAAAAACCTTTCCTCTACCTCAGTTCTCTTATCA
TGGGATCCCCCAGTAAAGCCAAATGGTGCAATAATAAGTTATGATTTAACTTTACAAGGACCAAATGAAAAT
TATTCTTTCATTACTTCTGATAATTACATAATATTCGAAGAGCTTTCACCATTTACATTATATAGCTTTTTT
GCTGCCGCAAGAACTAGAAAAGGACTTGGTCCTTCCAGTATTCTTTTCTTTTACACAGATGAGTCAGTGCCG
TTAGCACCTCCACAAAATTTGACTTTAATCAACTGTACTTCAGACTTTGTATGGCTCAAATGGAGCCCAAGT
CCTCTTCCAGGTGGTATTGTTAAAGTATATAGTTTTAAAATTCATGAACATGAAACTGACACTATATATTAT
AAGAATATATCAGGATTTAAAACTGAAGCCAAACTTGTTGGACTGGAACCAGTCAGCACCTACTCTATCCGT
GTATCTGCGTTCACCAAAGTTGGAAATGGCAATCAATTTAGTAATGTAGTAAAATTCACAACCCAAGAATCA
GTTCCAGATGTCGTGCAGAATATGCAGTGCATGGCAACTAGCTAACAGTCAGTTTTAGTGAAATGGGATCCA
CCCAAAAAGGCAAATGGAATAATAACGCAGTATATGGTAACAGTTGAAAGGAATTCTACAAAAGTTTCTCCC
CAAGATCACATGTACACTTTCATAAAGCTTCTTGCCAATACCTCATATGTCTTTAAAGTAAGAGCTTCAACC
TCAGCTGGTGAAGGTGATGAAAGCACATGCCATGTCAGCACACTACCTGAAACAGTTCCCAGTGTTCCCACA
AATATTGCTTTTTCTGATGTTCAGTCAACTAGTGCAACATTGACATGGATAAGACCTGACACTATCCTTGGC
TACTTTCAAAATTACAAAATTACCACTCAACTTCGTGCTCAAAAATGCAAAGAATGGGAATCCGAAGAATGT
GTTGAATATCAAAAAATTCAATACCTCTATGAAGCTCACTTAACTGAAGAGACAGTATATGGATTAAAGAAA
TTTAGATGGTATAGATTCCAAGTGGCTGCCAGCACCAATGCTGGCTATGCCAATGCTTCAAACTGGATTTCT
ACAAAAACTCTGCCTGGCCCTCCAGATGGTCCTCCTGAAAATGTTCATGTAGTAGCAACATCACCTTTTAGC
ATCAGCATAAGCTGGACTGAACCTGCTGTCATTACTGGACCAACATGTTATCTGATTGATGTCAAATCGGTA
GATAATGATCAATTTAATAThTCCTTCATCAAGTCAAATGAAGAAAATAAAACCATAGAAATTAAAGATTTA
GAAATATTCACAAGGTATTCTGTAGTGATCACTGCATTTACTCGGAACATTAGTGCTGCATATGTAGAAGGG
AAGTCAAGTGCTCAAATGATTGTTACTACTTTAGAATCAGCCCCAAAGGACCCACCTAACAACATCACATTT
CAGAAGATACCACATGAAGTTACAAAATTTCAATTAACGTCCCTTCCTCCTTCTCAACCTAATGGAAATATC
CAAGTATATCAAGCTCTGGTTTACCGAGAAGATGATCCTACTGCTGTCCACATTCACAACCTCAGTATTATA
CAGAAAACCAACACATTCGTCATTGCAATGCTAGAAGGACTAAAAGGTGGACATACATACAATATCAGTGTT
TACGCAGTCAATAGTGCTGGTGCAGGTCCAAAGGTTCCGATGAGAATAACCATGGATATCAAAGCTCCAGCA
CGACCAAAAACCAAACCAACCCCTATTTATGATGCCACAGGAAAACTGCTTGTGACTTCAACAACAATTACA
ATCAGAATGCCAATATGTTACTACAGTGATGATCATGGACCAATAAAAAATGTACAAGTGCTTGTGACAGAA
ACAGGAGCTCAGCATGATGGAAATGTAACAAAGTGGTATGATGCATATTTTAATAAAGCAAGGCCATATTTT
ACAAATGAAGGCTTTCCTAACCCTCCATGTACAGAAGGAAAGACAAAGTTTAGTGGCAATGAAGAAATCTAC
ATCATAGGTGCTGATAATGCATGCATGATTCCTGGCAATGAAGACAAAATTTGCAATGGACCACTGAAACCA
AAAAAGCAATACTTATTTAAATTTAOAGCTACAAATATTATGGGACAATTTACTGACTCTGATTATTCTGAC
CCTGTTAACACTTTAGGCGAAGGACTTTCAGAAAGAACCCTCGAG

The disclosed NOV2b polypeptide (SEQ ID NO: 8) encoded by SEQ ID NO: 7 has 855 amino acid residues and is presented in Table 2D using the one-letter amino acid code.

TABLE 2D
Encoded NOV2b protein sequence.

(SEQ ID NO:8)

RSPEGFVGNLTYESISSTAINVSWVPPAQPNGLVFYYVSLILQQTPRHVRPPLVTYERSIYFDNLEKYTDYI
LKITPSTEKGFSDTYTAQLYIKTEEDVPETSPIINTFKNLSSTSVLLSWDPPVKPNGATISYDLTLQGPNEN
YSFITSDNYIILEELSPFTLYSFFAAARTRKGLGPSSILFFYTDESVPLAPPQNLTLINCTSDFVWLKWSPS
PLPGGIVKVYSFKIHEHETDTIYYKNISGFKTEAKLVGLEPVSTYSIRVSAFTKVGNGNQFSNVVKFTTQES
VPDVVQNMQCMATSWQSVLVKWDPPKKANGIITQYMVTVERNSTKVSPQDHMYTFIKLLANTSYVFKVRAST
SAGEGDESTCHVSTLPETVPSVPTNIAFSDVQSTSATLTWIRPDTILGYFQNYKITTQLRAQKCKEWESEEC
VEYQKIQYLYEAHLTEETVYGLKKFRWYRFQVAASTNAGYGNASNWISTKTLPGPPDGPPENVHVVATSPFS
ISISWSEPAVITGPTCYLIDVKSVDNDEFNISFIKSNEENKTIEIKDLEIFTRYSVVITAFTGNISAAYVEG
KSSAEMIVTTLESAPKDPPNNMTFQKTPDEVTKFQLTSLPPSQPNGNIQVYQALVYREDDPTAVQIHNLSII
QKTNTFVIAMLEGLKGOHTYNISVYAVNSAGAGPKVPMRITMDIKAPARPKTKPTPIYDATGKLLVTSTTIT
IRMPICYYSDDHGPIKNVQVLVTETGAQHDGNVTKWYDAYFNXARPYFTNEGFPNPPCTEGKTKFSGNEEIY
IIGADNACMIPGNEDKICNGPLKPKKQYLFKFRATNIMGQFTDSDYSDPVKTLGEGLSERTLE

NOV2b is expressed in Brain, Colon, Fetal brain, Germ Cell, Heart, Kidney, Prostate, Uterus, brain, breast, colon, kidney, lung.

NOV2c

A disclosed NOV2c nucleic acid of 6903 nucleotides (also referred to as CG50718-05) encoding a novel Glomerular Mesangial Cell Receptor Protein-Tyrosine Phosphatase Precursor-like protein is shown in Table 2E. An open reading frame was identified beginning with an ATG initiation codon at nucleotides 1-3 and ending with a TGA codon at nucleotides 6901-6903. A putative untranslated regions upstream from the initiation codon and downstream of the termination codon are underlined in Table 2E. The start and stop codons are in bold letters.

TABLE 2E
NOV2c nucleotide sequence.

(SEQ ID NO:9)

| | |
|---|---|
| ATGGATTTTCTTATCATTTTTCTTTTACTTTTTATTGGGACTTCAGAGACACAGGTAGATGTTTCCAATGTC | |
| GTTCCTGGTACTAGGTACGATATAACCATCTCTTCAATTTCTACAACATACACCTCACCTGTTACTAGAATA | |
| GTGACAACAAATGTAACAGAACCAGGQCCTCCAGTCTTCCTAGCCGGGGAAAGAGTCGGATCTGCTGGGATT | |
| CTTCTGTCTTGGAATACACCACCTAATCCAAATGGAAGGATTATATCTTACATTGTCAAATATAAGGAAGTT | |
| TGTCCGTGGATGCAAACAGTATATACACAAGTCAGATCAAAGCCAGACAGTCTGGAAGTTCTTCTTACTAAT | |
| CTTAATCCTGGAACAACATATGAAATTAAGGTAGCTGCTGAAAACAGTGCTGGCATTGGAGTGTTTAGTGAT | |
| CCATTTCTCTTCCAAACTGCAGAAAGTCCAGCTCCAGGAAAAGTGCTGAATCTCACAGTTGAGGCCTACAAC | |
| GCTTCAGCAGTTAAGCTGATTTGGTATTTACCTCGGCAACCAAATGGCAAAATTACCAOCTTCAAGATTAGT | |
| GTCAAGCATGCCAGAAGTGGGATACTAGTGAAAGATGTCTCAATCAGAGTAGAGGACATTTTGACTGGGAAA | |
| TTGCCAGAATGCAATGTAGAGAATAGTGAATCTTTTTTATGGAGTACAGCCAGCCCTTCTCCAACCCTTGGT | |
| AGAGTTACACCTCCATCGCGTACCACACATTCATCAAGCACGTTGACACAGAATGAGATCAGCTCTGTGTGG | |
| AAAGAGCCTATCAGTTTTGTAGTGACACACTTGAGACCTTATACAACATATCTTTTTGAAGTTTCAGCTGCT | |
| ACAACTGAAGCAGGTTATATTGATAGTACGATTGTCAGAACACCAGAATCAGTGCCTGAAGGACCACCACAA | |
| AACTCCGTAACAGGCAACATCACAGGAAAGTCCTTTTCAATTTTATGGGACCCACCAACTATAGTAACAGGG | |
| AAATTTAGTTATAGAGTTGAATTATATGGACCATCAGGTCGCATTTTGGATAACAGCACAAAAGACCTCAAG | |
| TTTGCATTCACTAACCTAACACCATTTACAATCTATGATGTCTATATTGCGGCTGAAACCAGTGCAGGGACT | |
| GGGCCCAAGTCAAATATTTCAGTATTCACTCCACCAGATGTTCCAGGGGCAGTGTTTGATTTACAACTTGCA | |
| GAGGTAGAATCCACGCAAGTAAGAATTACTTGGAAGAAACCACGACAACCAAATGGAATTATTAACCAATAC | |
| CGAGTGAAAGTGCTAGTTCCAGAGACAGGAATAATTTTGGAAAATACTTTGCTCACTGGAAATAATGAGATA | |
| AATGACCCCATGGCTCCAGAAATTGTGAACATAGTAGAGCCAATGGTAGGATTATATGAGGGTTCAGCAGAG | |
| ATGTCGTCTGACCTTCACTCACTTCCTACATTTATATATAACAGCCATCCAGATAAAAACTTTCCTGCAAGG | |
| AATAGAGCTGAAGACCAGACTTCACCAGTTGTAACTACAAGGAATCAGTATATTACTGACATTGCAGCTGAA | |
| CAGCTGTCTTATGTTATCAGGAGACTTGTACCTTTCACTGAGCACATGATTAGTGTATCTGCTTTCACCATC | |
| ATGGGAGAAGGACCACCAACAGTTCTCAGTGTTAGGACACGTCAGCAAGTGCCAAGCTCCATTAAAATTATA | |
| AACTATAAAAATATTAGTTCTTCATCTATTTTGTTATATTGGGATCCTCCAGAATATCCCAATGGAAAAATA | |
| ACTCACTATACGATTTATGCAATGGAATTGGATACAAACAGAGCATTCCAGATAACTACCATAGATAACAGC | |
| TTTCTCATAACAGGTATAGGGTTAAAGAAATACACAAAATACAAAATGAGAGTGGCAGCCTCAACCCACGTT | |
| GGAGAAAGTTCTTTGTCTGAAGAAAATGACATCTTTGTGAGAACTTCAGAAGATGAACCGGAATCATCACCT | |
| CAAGATGTCGAAGTAATTGATGTTACCGCAGATGAAATAAGGTTGAAGTGGTCACCACCCGAAAACCCCAAT | |
| GGGATCATTATTGCTTATGAAGTGCTATATAAAAATATAGATACTTTATATATGAAGAACACATCAACAACA | |
| GACATAATATTAAGGAACTTAAGACCTCACACCCTCTATAACATTTCTGTAAGGTCTTACACCAGATTTGGT | |
| CATCGCAATCAGGTATCTTCTTTACTCTCTGTAAGGACTTCGGAGACTGTGCCTGATAGTGCACCAGAAAAT | |
| ATCACTTACAAAAATATTTCTTCTGGAGAGATTGAGCTATCATTCCTTCCCCCAAGTAGTCCCAATGGAATC | |
| ATACAAAAATATACAATTTATCTCAAGAGAAGTAATGGAAATGAGGAAAGAACTATAAATACAACCTCTTTA | |
| ACCCAAAACATTCTGAAGAAATATACCCAATATATCATTGAGGTGTCTGCTAOTACACTCAAAGGTGAAGGA | |
| GTTCGGAGTGCTCCCATAAGTATACTGACGGAGGAAGATGCTCCTGATTCTCCCCCTCAACACTTCTCTGTA | |
| AAACAGTTGTCTGGTGTCACGGTGAAGTTGTCATGGCAACCACCCCTGGAGCCAAATGCAATTATCCTTTAT | |
| TACACAGTTTATGTCTGGAGGAATAGATCATCATTAAAAACTATTAATGTCACTGAAACATCATTGGAGTTA | |
| TCAGATTTGGATTATAATGTTCAATACAGTGCTTATGTAACAGCTAGCACCAGATTTGGTGATGGGAAAACA | |
| AGAAGCAATATCATTACCTTTCAAACACCAGAGGGACCAAGCGATCCTCCCAAAGATGTTTATTATGCAAAC | |
| CTCAGTTCTTCATCAATAATTCTTTTCTGGACACCTCCTTCAAAACCTAATGGGATTATACAATATTACTCT | |
| GTTTATTACAGAAATACTTCAGGTACTTTTATGCAGAATTTTACACTCCATGAAGTAACCAATGACTTTGAC | |
| AATATGACTGTATCCACAATTATAGATAAACTGACAATATTCAGCTACTATACATTTTCGTTAACAGCAAGT | |
| ACTTCAGTTGGAAATGGGAATAAAAGCAGTGACATCATTGAAGTATACACAGATCAAGACGTCCCTGAAGGG | |
| TTTGTTGGAAACCTGACTTACGAATCCATTTCGTCAACTGCAATAAATGTAACCTGGGTCCCACCCGCTCAA | |
| CCAAACGGTCTAGTCTTCTACTATGTTTCACTGATCTTACAGCAGACTCCTCGCCATGTGAGACCACCTCTT | |
| GTTACATATGAGAGAAGCATATATTTTGATAATCTCGAAAAATACACTGATPATATATTAAAAATTACTCCA | |
| TCAACAGAAAAGGGATTCTCTGAThCCTATACTGCCCAGCTATACATCAAGACTGAAGAAGATGTCCCAGAA | |
| ACTTCACCAATAATCAACACTTTTAAAAACCTTTCCTCTACCTCAGTTCTCTTATCATGGGATCCCCCAGTA | |
| AAGCCAAATGGTGCAATAATAAGTTATGATTTAACTTTACAAGGACCAAATGAAAATTATTCTTTCATTACT | |
| TCTGATAATTACATAATATTGGAAGAGCTTTCACCATTTACATTATATAGCTTTTTTGCTGCCGCAAGAACT | |
| AGAAAAGGACTTGGTCCTTCCAGTATTCTTTTCTTTTACACAGATGAGTCAGTGCCGTTAGCACCTCCACAA | |
| AATTTGACTTTAATCAACTGTACTTCAGACTTTGTATGGCTGAAATGGAGCCCAAGTCCTCTTCCAGGTGGT | |
| ATTGTTAAAGTATATAGTTTTAAAATTCATGAACATGAAACTGACACTATATATTATAAGAATATATCAGGA | |
| TTTAAAACTGAAGCCAAACTTGTTGGACTGGAACCAGTCAGCACCTACTCTATCCGTGTATCTGCGTTCACC | |
| AAAGTTGGAAATGGCAATCAATTTAGTAATGTAGTAAAATTCACAACCCAAGAATCAGTTCCAGATGTCGTG | |
| CAGAATATGCAGTGCATGGCAACTAGCTGGCAGTCAGTTTTAGTGAAATGGGATCCACCCAAAAAGGCAAAT | |
| GGAATAATAACGCAGTATATGGTAACAGTTGAAAGGAATTCTACAAAAGTTTCTCCCCAAGATCACATGTAC | |
| ACTTTCATAAAGCTTCTTGCCAATACCTCATATGTCTTTAAAGTAAGAGCTTCAACCTCAGCTGGTGAAGGT | |
| GATGAAAGCACATGCCATGTCAGCACACTACCTGAAACAGTTCCCAGTGTTCCCACAAATATTGCTTTTTCT | |
| GATGTTCAGTCAACTAGTGCAACATTGACATGGATAAGACCTGACACTATCCTTGGCTACTTTCAAAATTAC | |
| AAAATTACCACTCAACTTCGTGCTCAAAAATGCAAAGAATGGGAATCCGAAGAATGTGTTGAATATCAAAAA | |
| ATTCAATACCTCTATGAAGCTCACTTAACTGAAGAGACAGTATATGGATTAAAGAAATTTAGATGGTATAGA | |
| TTCCAAGTGGCTGCCAGCACCAATGCTGGCTATGGCAATGCTTCAAACTGGATTTCTACAAAAACTCTGCCT | |
| GGCCCTCCAGATGGTCCTCCTGAAAATGTTCATGTAGTAGCAACATCACCTTTTACCATCAGCATAAGCTGG | |
| AGTGAACCTGCTGTCATTACTGGACCAACATGTTATCTGATTGATGTCAAATCGGTAGATAATGATGAATTT | |
| AATATATCCTTCATCAAGTCAAATGAAGAAAATAAAACCATAGAAATTAAAGATTTAGAAATATTCACAAGG | |
| TATTCTGTAGTGATCACTGCATTTACTGGGAACATTAGTGCTGCATATGTAGAAGGGAAGTCAAGTGCTGAA | |
| ATGATTGTTACTACTTTAGAATCAGCCCCAAAGGACCCACCTAACAACATGACATTTCAGAAGATACCAGAT | |
| CAAGTTACAAAATTTCAATTAACGTCCCTTCCTCCTTCTCAACCTAATGGAAATATCCAAGTATATCAAGCT | |
| CTGGTTTACCGAGAAGATGATCCTACTGCTGTCCAGATTCACAACCTCAGTATTATACAGAAAACCAACACA | |
| TTCGTCATTGCAATGCTAGAAGGACTAMAGGTGGACATACATACAATATCAGTGTTTACGCAGTCAATAGAA | |
| GCTGGTGCAGGTCCAAAGGTTCCGATGAGAATAACCATGGATATCAAAGCTCCAGCACGACCAAAAACCAAA | |
| CCAACCCCTATTTATGATGCCACAGGAAAACTGCTTGTGACTTCAACAACAATTACAATCAGAATGCCAATA | |
| TGTTACTACAGTGATGATCATGGACCAATAAAAAATGTACAAGTGCTTCTGACAGAAACAGGACCTCAGCAT | |
| GATGGAAATGTAACAAAGTGGTATGATGCATATTTTAATAAAGCAAGGCCATATTTTACAAATGAAGGCTTT | AA |
| CCTAACCCTCCATGTACAGAAGGAAAGACAAAGTTTAGTGGCAATGAAGAAATCTACATCATAGGTGCTGAT | |
| AATGCATGCATGATTCCTGGCAATGAAGACAAAATTTGCAATGGACCACTGAAACCAAAAAAGCAATACTTA | |
| TTTAAATTTAGAGCTACAAATATTATGGGACAATTTACTGACTCTGATTATTCTGACCCTGTTAAGACTTTA | |
| GGCGAAGCACTTTCAGAAAGAACCCTAGAGATCATTCTTTCCGTCACTTTGTGTATCCTTTCAATAATTCTC | |
| CTTGGAACAGCTATTTTTGCATTTGCAAGAATTCGACAGAAGCAGAAAGAAGGTGGCACATACTCTCCTCAG | |
| GATGCAGAAATTATTGACACTAAATTGAAGCTGGATCAGCTCATCACAGTGGCAGACCTGGAACTGAAGGAC | |
| GAGAGATTAACGCGGTTACTTAGTTATAGAAAATCCATCAAGCCAATAAGCAAGAAATCCTTCCTGCAACAT | |
| GTTGAAGAGCTTTGCACAAACAACAACCTAAAGTTTCAAGAAGAATTTTCGGAATTACCAAAATTTCTTCAG | |
| GATCTTTCTTCAACTGATGCTGATCTGCCTTGGAATAGAGCAAAAAACCGCTTCCCAAACATAAAACCATAT | |
| AATAATAACAGAGTAAAGCTGATAGCTGACGCTAGTGTTCCAGGTTCGGATTATATTAATGCCAGCTATATT | |
| TCTGGTTATTTATGTCCAAATGAATTTATTGCTACTCAAGGTCCACTACCAAAAACAGTTGGAGATTTTTAA | |
| AGAATGGTGTGGGAAACCAGAGCAAAAACATTAGTAATGCTAACACAGTGTTTTGAAAAAGGACGGATCAGA | |
| TGCCATCAGTATTCGCCAGAGGACAACAAGCCAGTTACTGTCTTTGGAGATATAGTGATTACAAGCTIAATG | |
| GAGGATGTTCAAATAGATTGGACTATCAGGGATCTGAAAATTGAAAGGCATGGCGATTGCATGACTGTTCGA | |
| CAGTGTAACTTTACTGCCTGGCCAGAGCATGGGGTTCCTCAGAACAGCGCCCCTCTAATTCACTTTGTGAAG | |
| TTGGTTCGAGCAAGCAGGGCACATGACACCACACCTATGATTGTTCACTGTAGTGCTGGAGTTGGAAGAACT | |
| GGAGTTTTTATTGCTCTGGACCATTTAACACAACATATAAATGACCATGATTTTGTGGATATATATGGACTA | |
| GTACCTGAACTGAGAAGTGAAAGAATGTGCATGGTGCAGAATCTGGCACAGTATATCTTTTTACACCAGTGC | |
| ATTCTGGATCTCTTATCAATAAAGGGAAGTATCAGCCCATCTGTTTTGTTAACTATTCAGCACTTCAGPAAG | |
| ATGGACTCTTTGGACGCCATGGAAGGTGATGTTCAGCTTGAATGGG1XAGAACCACTATGTAA | |

In a search of public sequence databases, the NOV2c nucleic acid sequence, located on chromsome 12 has 5903 of 6906 bases (85%) identical to a gb:GENBANK-ID:AF063249|acc:AF063249.1 mRNA from Rattus norvegicus (Rattus norvegicus glomerular mesangial cell receptor protein-tyrosine phosphatase precursor (PTPRQ) mRNA, complete cds) (E=0.0). Public nucleotide databases include all GenBank databases and the GeneSeq patent database.

The disclosed NOV2c polypeptide (SEQ ID NO: 10) encoded by SEQ ID NO: 9 has 2300 amino acid residues and is presented in Table 2F using the one-letter amino acid code. Signal P, Psort and/or Hydropathy results predict that NOV2c has a signal peptide and is likely to be localized in the plasma membrane with a certainty of 0.4600. In other embodiments, NOV2c may also be localized to the microbody (peroxisome) with acertainty of 0.1260, the endoplasmic reticulum (membrane) with a certainty of 0.1000 or in the endoplasmic reticulum (lumen) with a certainty of 0.1000. The most likely cleavage site for a NOV2c peptide is between amino acids 17 and 18, at: SET-QV.

TABLE 2F
Encoded NOV2c protein sequence.

(SEQ ID NO:10)

MDFLIIFLLLFIGTSETQVDVSNVVPGTRYDITISSISTTYTSPVTRIVTTNVTEPGPPVFLAGERVGSAGI
LLSWNTPPNPNGRIISYIVKYKEVCPWMQTVYTQVRSKPDSLEVLLTNLNPGTTYEIKVAAENSAGIGVFSD
PFLFQTAESPAPGKVVNLTVEAYNASAVKLIWYLPRQPNGKITSFKISVKHARSGIVVKDVSIRVEDILTGK
LPECNVENSESFLWSTASPSPTLGRVTPPSRTTHSSSTLTQNEISSVWKEPISFVVTHLRPYTTYLFEVSAA
TTEAGYIDSTIVRTPESVPEGPPQNCVTGNITGKSFSILWDPPTIVTGKFSYRVELYGPSGRILDNSTKDLK
FAFTNLTPFTMYDVYIAAETSAGTGPKSNISVFTPPDVPGAVFDLQLAEVESTQVRITWKKPRQPNGIINQY
RVKVLVPETGIILENTLLTGNNETNDPMAPEIVNIVEPMVGLYEGSAEMSSDLHSLATFIYNSHPDKNFPAR
NRAEDQTSPVVTTRNQYITDIAAEQLSYVIRRLVPFTEHMISVSAFTIMGEGPPTVLSVRTRQQVPSSIKII
NYKNISSSSILLYWDPPEYPNGKITHYTIYAMELDTNRAFQITTIDNSFLITGIGLKKYTKYKMRVAASTHV
GESSLSEENDIFVRTSEDEPESSPQDVEVIDVTADEIRLKWSPPEKPNGIIIAYEVLYKNIDTLYMKNTSTT
DIILRNLRPHTLYNISVRSYTREGHGNQVSSLLSVRTSETVPDSAPENITYKNISSGEIELSFLPPSSPNGI
IQKYTIYLKRSNGNEERTINTTSLTQNILKKYTQYIIEVSASTLKGEGVRSAPISILTEEDAPDSPPQDFSV
KQLSGVTVKLSWQPPLEPNGIILYYTVYVWRNRSSLKTINVTETSLELSDLDYNVEYSAYVTASTRFGDGKT
RSNIISFQTPEGPSDPPKDVYYANLSSSSIILFWTPPSKPNGIIQYYSVYYRNTSGTFMQNFTLHEVTHDFD
NMTVSTIIDKLTIFSYYTFWLTASTSVCNGNKSSDIIEVYTDQDVPEGFVGNLTYESISSTAINVSWVPPAQ
PNGLVFYYVSLILQQTPRHVRPPLVTYERSIYFDNLEKYTDYILKITPSTEKGFSDTYTAQLYIKTEEDVPE
TSPIINTFKNLSSTSVLLSWDPPVKPNGAIISYDLTLQGPNENYSFITSDNYIILEELSPFTLYSFFAAART
RKGLGPSSILFFYTDESVPLAPPQNLTLINCTSDFVWLKWSPSPLPGGIVKVYSFKIHEHETDTIYYKNISG
FKTEAKLVGLEPVSTYSIRVSAETKVGNGNQFSNVVKFTTQESVPDVVQNMQCMATSWQSVLVKWDPPKKAN
GIITQYMVTVERNSTKVSPQDHMYTFIKLLANTSYVFKVRASTSAGEGDESTCHVSTLPETVPSVPTNIAFS
DVQSTSATLTWIRPDTILGYFQNYKITTQLRAQKCKEWESEECVEYQKIQYLYEAHLTEETVYGLKKFRWYR
FQVAASTNAGYGNASNWISTKTLPGPPDGPPENVHVVATSPFSISISWSEPAVITGPTCYLIDVKSVDNDEF
NISFIKSNEENKTIEIKDLEIFTRYSVVITAFTGNISAAYVEGKSSAEMIVTTLESAPKDPPNNMTFQKIPD
EVTKEQLTSLPPSQPNGNIQVYQALVYREDDPTAVQIHNLSIIQKTNTFVIAMLEGLKGGHTYNISVYAVNS
AGAGPKVPMRITMDIKAPARPKTKPTPIYDATGKLLVTSTTITIRMPICYYSDDHGPIKNVQVLVTETGAQH
DGNVTKWYDAYFNKARPYFTNEGFPNPPCTEGKTKFSGNEETYIIGADNACMIPCNEDKICNGPLKPKKQYL
FKFRATNIMGQFTDSDYSDPVKTLGEGLSERTLEIILSVTLCILSIILLGTAIFAFARIRQKQKEGGTYSPQ
DAEIIDTKLKLDQLITVADLELKDERLTRLLSYRKSIKPISKKSFLQHVEELCTNNNLKFQEEFSELPKFLQ
DLSSTDADLPWNRAKNRFPNIKPYNNNRVKLIADASVPGSDYINASYTSGYLCPNEFIATQGPLPGTVGDFW
RMVWETRAKTLVMLTQCFEKGRIRCHQYWPEDNKPVTVFGDIVITKLMEDVQIDWTIRDLKIERHGDCMTVR
QCNFTAWPEHGVPENSAPLIHFVKLVRASRAHDTTPMIVHCSAGVGRTGVFIALDHLTQHINDHDFVDIYGL
VAELRSERMCMVQNLAQYIFLHQCILDLLSNKGSNQPICFVNYSALQKMDSLDAMEGDVELEWEETTM

A search of sequence databases reveals that the NOV2c amino acid sequence has 1988 of 2301 amino acid residues (86%) identical to, and 2151 of 2301 amino acid residues (93%) similar to, the 2302 amino acid residue ptnr:SPTREMBL-ACC:088488 protein from Rattus norvegicus (Rat) (Glomerular Mesangial Cell Receptor Protein-Tyrosine Phosphatase Precursor (EC 3.1.3.48)) (E=0.0). Public amino acid databases include the GenBank databases, SwissProt, PDB and PIR.

NOV2c is expressed in at least Synovium/Synovial membrane, Kidney. Expression information was derived from the tissue sources of the sequences that were included in the derivation of the sequence of CuraGen Acc. No. CG50718-05. The sequence is predicted to be expressed in the Rattus norvegicus: glomerular mesangial. because of the expression pattern of (GENBANK-ID: gb:GENBANK-ID:AF063249|acc:AF063249.1) a closely related Rattus norvegicus glomerular mesangial cell receptor protein-tyrosine phosphatase precursor (PTPRQ) mRNA, complete cds homolog.

Homologies among each of the above NOV2 proteins will be shared by the other NOV2 proteins insofar as they are homologous to each other as shown below in Table 2G. Any reference to NOV2 is assumed to refer to all three of the NOV2 proteins in general, unless otherwise noted.

[image]

[image]

[image]

[image]

The disclosed NOV2a polypeptide has homology to the amino acid sequences shown in the BLASTP data listed in Table 2H.

TABLE 2H
BLAST results for NOV2a

| Gene Index/ | | | Identity | Positives | |
|---|---|---|---|---|---|
| Identifier | Protein/Organism | Length (aa) | (%) | (%) | Expect |
| gi\|12621078\|ref\|NP_{—} | protein tyrosine | 2302 | 1893/2306 | 2077/2306 | 0.0 |
| 075214.1\| | phosphatase, | | (82%) | (89%) | |
| (NM_022925) | receptor type, Q | | | | |
| | [Rattus | | | | |
| | norvegicus] | | | | |
| gi\|125977\|sp\|P16621\| | PROTEIN-TYROSINE | 2029 | 410/1587 | 680/1587 | 1e−94 |
| LAR_DROME | PHOSPHATASE DLAR | | (25%) | (42%) | |
| | PRECURSOR | | | | |
| | (PROTEIN- | | | | |
| | TYROSINE- | | | | |
| | PHOSPHATE | | | | |
| | PHOSPHOHYDROLASE) | | | | |
| gi\|10728878\|gb\|AAF53837.2\| | Lar gene product | 2037 | 410/1587 | 680/1587 | 2e−94 |
| (AE003663) | [Drosophila | | (25%) | (42%) | |
| | melanogaster] | | | | |
| gi\|7290546\|gb\|AAF45998.1\| | Ptp4E gene | 1767 | 417/1645 | 694/1645 | 8e−94 |
| (AE003432) | product | | (25%) | (41%) | |
| | [Drosophila | | | | |
| | melanogaster] | | | | |
| gi\|1362625\|pir\|\|A49502 | protein-tyrosine- | 1767 | 416/1645 | 693/1645 | 1e−92 |
| | phosphatase (EC | | (25%) | (41%) | |
| | 3.1.3.48), | | | | |
| | receptor type 4E, | | | | |
| | splice form A | | | | |
| | precursor - fruit | | | | |
| | fly (Drosophila | | | | |
| | melanogaster) | | | | |

The homology between these and other sequences is shown graphically in the ClustalW analysis shown in Table 2I. In the ClustalW alignment of the NOV2 proteins, as well as all other ClustalW analyses herein, the black outlined amino acid residues indicate regions of conserved sequence (i.e., regions that may be required to preserve structural or functional properties), whereas non-highlighted amino acid residues are less conserved and can potentially be altered to a much broader extent without altering protein structure or function.

[image]

[image]

[image]

[image]

[image]

[image]

[image]

[image]

Tables 2J-2EE list the domain descriptions from DOMAIN analysis results against NOV2a. This indicates that the NOV2a sequence has properties similar to those of other proteins known to contain this domain.

TABLE 2J
Domain Analysis of NOV2a

gn1|Smart|smart00194, PTPc, Protein tyrosine phosphatase, catalytic
domain (SEQ ID NO:93)
CD-Length = 264 residues, 99.6% aligned
Score = 318 bits (816), Expect = 2e−87

| | | | |
|---|---|---|---|
| NOV 1: | 1983 | KFQEEFSELPK-FLQDLSSTDADLPWNRAKNRFPNIKPYNNNRVKLIADASVPGSDYINA | 2041 |
| | | +\|\|\| +\| + \|\|\| \| \| \|\| \|\| \|\|\|+ ++ \|\|++ \|\|\|\| \|\|\|\|\|\|\| | |
| Sbjct: | 1 | GLEEEFEKLQRLTPDDLSCTVAILPENRDKNRYKDVLPYDHTRVKL-KPPPGEGSDYINA | 59 |
| NOV 1: | 2042 | SYISGYLCPNEFIATQGPLPGTVGDFWRMVWETRAKTLVMLTQCFEKGRIRCHQYWPEDN | 2101 |
| | | \|\|\| \| \| +\|\|\|\|\|\|\|\| \|\| \|\|\|\|\|\|\|\| + +\|\|\|\|+ \|\|\|\| +\| \|\|\|\|\| | |
| Sbjct: | 60 | SYIDGPNRPKAYIATQGPLPSTVEDFWRMVWEEKVPVIVMLTELVEKGREKCAQYWPEKE | 119 |
| NOV 1: | 2102 | KPVTVFGDIVITKLMEDVQIDWTIRDLKIERHG--DCMTVRQCNFTAWPEHGVPENSAPL | 2159 |
| | | +\|\|\| +\| + \|+\|\|\| \|++ \| + \|\| ++\| \|\|+\|\|\|\|\|+ \| | |
| Sbjct: | 120 | GGSLTYGDITVTLKSVEKVDDYTIRTLEVTNTGCSETRTVTHYHYTNWPDHGVPESPKSL | 179 |
| NOV 1: | 2160 | IHFVKLVRASRAH--DTTPMIVHCSAGVGRTGVFIALDHLTQHINDHDFVDIYGLVAELR | 2217 |
| | | + \|+ \|\| \|++ ++ \|++\|\|\|\|\|\|\|\|\|\|\| \|\|\|+\| \| \| + \|\|\|+ +\| \|\|\| | |
| Sbjct: | 180 | LDLVRAVRKSQSTLRNSGPIVVHCSAGVORTGTFIAIDILLQQLEAGKEVDIFEIVICELR | 239 |
| NOV 1: | 2218 | SERMCMVQNLAQYIFLHQCILDLL 2241 | |
| | | \|+\| \|\|\| \|\|\|\|\|++ \|\|+ \| | |
| Sbjct: | 240 | SQRPGMVQTEEQYIFLYRAILEYL 263 | |

TABLE 2K

Domain Analysis of NOV2a

gnl|Pfam|pfam00102, Y_phosphatase, Protein-tyrosine phosphatase (SEQ ID NO:94)
CD-Length = 235 residues, 100.0% aligned
Score = 275 bits (704), Expect = 2e−74

| | | | |
|---|---|---|---|
| NOV1: | 2008 | NRAKNRFPNIKPYNNNRVKLIADASVPGSDYINASYISGYLCPNEFIATQGPLPGTVGDF | 2067 |
| | | \|+ \|\|\|+ ++ \|\|++ \|\|\|\| \|\|\|\|\|\|\|\|+ \|\| \| +\|\|\|\|\|\|\|\| \|+ \|\| | |
| Sbjct: | 1 | NKEKNRYKDVLPYDHTRVKL-KPLGDEDSDYINASYVDGYKKPKAYIATQGPLPNTIEDF | 59 |
| NOV1: | 2068 | WRMVWETRAKTLVMLTQCFEKGRIRCHQYWPEDNKPVTVFGDI-VITKLMEDVQIDWTIR | 2126 |
| | | \|\|\|\|\|\| + + +\|\|\|\|+ \|\|\|\| +\| \|\|\|\|\| +\|\| \| +\| + \|+\|+\| | |
| Sbjct: | 60 | WRMVWEEKVRVIVMLTELVEKGREKCAQYWPEKEGGSLTYGDFTVTCVSVEKKKDDYTVR | 119 |
| NOV1: | 2127 | DLKIERHGDC--MTVRQCNFTAWPEHGVPENSAPLIHFVKLVRASRAH-DTTPMIVHCSA | 2183 |
| | | \|++ \|\| \|\|+ ++\| \|\|+\|\|\|\|\|+ ++ ++ \|\| \|+ \| \|++\|\|\|\|\| | |
| Sbjct: | 120 | TLELTNSGDDETRTVKHYHYTGWPDHGVPESPKSILDLLRKVRKSKGTPDDGPIVVHCSA | 179 |
| NOV1: | 2184 | GVGRTGVFIALDHLTQHINDHDFVDIYGLVAELRSERMCMVQNLAQYIFLHQCILD | 2239 |
| | | \|+\|\|\|\| \|\|\|+\| \| \| + \|\|++ \| +\|\|\|+\| \|\|\| \|\|\|\|++ \|\|+ | |
| Sbjct: | 180 | GIGRTGTFIAIDILLQQLEKEGVVDVFDTVKKLRSQRPGMVQTEEQYTFIYDAILE | 235 |

TABLE 2L

Domain Analysis of NOV2a

gnl|Smart|smart00404, PTPc_motif, Protein tyrosine phosphatase, catalytic
domain motif (SEQ ID NO:95)
CD-Length = 105 residues, 100.0% aligned
Score = 120 bits (301), Expect = 8e−28

| | | | |
|---|---|---|---|
| NOV 1: | 2138 | TVRQCNFTAWPEHGVPENSAPLIHFVKLVRASRAH--DTTPMIVHCSAGVGRTGVFIALD | 2195 |
| | | \|\|+ ++\| \|\|+\|\|\|\|\|+ ++ \|++ \|+ \| + \|++\|\|\|\|\|\|\|\|\|\|\| \|+\|+\| | |
| Sbjct: | 1 | TVKHYHYTGWPDHGVPESPDSILEFLRAVKKSLNKSANNGPVVVHCSAGVGRTGTFVAID | 60 |
| NOV 1: | 2196 | HLTQHI-NDHDFVDIYGLVAELRSERMCMVQNLAQYIFLHQCILD | 2239 |
| | | \| \| + \|\|\|+ +\| \|\|\|\|+\| \|\| \| \|\|+\|\|++ +\|+ | |
| Sbjct: | 61 | ILLQQLEAGTGEVDIFDIVKELRSQRPGAVQTLEQYLFLYRALLE | 105 |

TABLE 2M

Domain Analysis of NOV2a

gnl|Pfam|pfam00041, fn3, Fibronectin type III domain (SEQ ID NO:96)
CD-Length = 86 residues, 100.0% aligned
Score = 60.8 bits (146), Expect = 8e−10

| | | | |
|---|---|---|---|
| NOV 1: | 54 | PGPPVFLAGERVGSAGILLSWNTPPNPNGRIISYIVKYKEVCPWMQTVYTQVRSKPDSLE | 113 |
| | | \| \| \| \| \| + \|\|\|+ \|\|+ \|\| \| \| \|+\|+ \| + ++ + | |
| Sbjct: | 1 | PSAPTNLTVTDVTSTSLTLSWSPPPDGNGPITGYEVEYQPVNS--GEEWNEITVPGTTTS | 58 |
| NOV 1: | 114 | VLLTNLNPGTTYEIKVAAENSAGIGVFS 141 | |
| | | \|\| \| \|\|\| \|\|++\| \| \| \| \| \| | |
| Sbjct: | 59 | YTLTGLKPGTEYEVRVQAVNGGGNGPPS 86 | |

TABLE 2N

Domain Analysis of NOV2a

gnl|Pfam|pfam00041, fn3, Fibronectin type III domain (SEQ ID NO:96)
CD-Length = 86 residues, 95.3% aligned
Score = 58.9 bits (141), Expect = 3e−09

| | | | |
|---|---|---|---|
| NOV 1: | 659 | SSPQDVEVIDVTADEIRLKWSPPEKPNGIIIAYEVLYKNIDTLYMKNT-----STTDIIL | 713 |
| | | \|+\| ++ \| \|\|\|+ + \| \|\|\|\| \|\| \| \|\|\| \|+ +++ \| +\|\| \| | |
| Sbjct: | 2 | SAPTNLTVTDVTSTSLTLSWSPPPDGNGPITGYEVEYQPVNSGEEWNEITVPGTTTSYTL | 61 |
| NOV 1: | 714 | RNLRPHTLYNISVRSYTRFGHG 735 | |
| | | \|+\| \| \| + \|++ \|+\| | |
| Sbjct: | 62 | TGLKPGTEYEVRVQAVNGGGNG 83 | |

TABLE 2O

Domain Analysis of NOV2a

gnl|Pfam|pfam00041, fn3, Fibronectin type III domain (SEQ ID NO:96)
CD-Length = 86 residues, 100.0% aligned
Score = 57.0 bits (136), Expect = 1e−08

| | | | |
|---|---|---|---|
| NOV 1: | 1330 | PDVVQNMQCMATSWQSVLVKWDPPKKANGIITQYMVTV-------ERNSTKVSPQDHMYT | 1382 |
| | | \| \|+ + \|+ + \| \|\| \|\| \|\| \| \| \| \| \| \|\| | |
| Sbjct: | 1 | PSAPTNLTVTDVTSTSLTLSWSPPPDGNGPITGYEVEYQPVNSGEEWNEITVPGTTTSYT | 60 |
| NOV1: | 1383 | FIKLLANTSYVFKVRASTSAGEGDES 1408 | |
| | | \| \| \| +\|+\| \| \| \| | |
| Sbjct: | 61 | LTGLKPGTEYEVRVQAVNGGGNGPPS 86 | |

TABLE 2P

Domain Analysis of NOV2a

gnl|Pfam|pfam00041, fn3, Fibronectin type III domain (SEQ ID NO:96)
CD-Length = 86 residues, 98.8% aligned
Score = 53.1 bits (126), Expect = 2e−07

| | | | |
|---|---|---|---|
| NOV 1: | 753 | SAPENITYKNISSGEIELSFLPPSSPNGIIQKYTIYLKRSNGNE---ERTINTTSLTQNI | 809 |
| | | \|\|\| \|+\| +++\| + \|\|+ \|\| \|\| \| \| + + \| \| \| \|+ \|+ + + | |
| Sbjct: | 2 | SAPTNLTVTDVTSTSLTLSWSPPPDNGPITGYEVEYQPVNSGEEWNEITVPGTTTSYTL | 61 |
| NOV 1: | 810 | KGLKKYTQYIIEVSASTLKGEGVRS 834 | |
| | | \|\|\| \|+\| + \| \| \| \| \| | |
| Sbjct: | 62 | TGLKPGTEYEVRVQAVNGGGNGPPS 86 | |

TABLE 2Q

Domain Analysis of NOV2a

gnl|Pfam|pfam00041, fn3, Fibronectin type III domain (SEQ ID NO:96)
CD-Length = 86 residues, 95.3% aligned
Score = 52.4 bits (124), Expect = 3e−07

| | | | |
|---|---|---|---|
| NOV 1: | 848 | SPPQDFSVKQLSGVTVKLSWQPPLEPNGIILYYTVYVWR----SSLKTINV--TETSLEL | 901 |
| | | \| \| + +\| ++ ++ \|\|\| \|\| + \|\| \| \| \| \| \| \| \|\| \| | |
| Sbjct: | 2 | SAPTNLTVTDVTSTSLTLSWSPPPDGNGPITGYEVEYQPVNSGEEWNEITVPGTTTSYTL | 61 |
| NOV 1: | 902 | SDLDYNVEYSAYVTASTRFGDG 923 | |
| | | + \| \|\| \| \| \|+\| | |
| Sbjct: | 62 | TGLKPGTEYEVRVQAVNGGGNG 83 | |

TABLE 2R

Domain Analysis of NOV2a

gnl|Pfam|pfam00041, fn3, Fibronectin type III domain (SEQ ID NO:96)
CD-Length = 86 residues, 91.9% aligned
Score = 51.6 bits (122), Expect = 5e−07

| | | | |
|---|---|---|---|
| NOV 1: | 1148 | TFKNLSSTSVLLSWDPPVKPNGAIISYDLTLQGPNENYSFIT-----SDNYIILEELSPF | 1202 |
| | | \| +++\|\|\|+ \|\|\| \|\| \|\| \| \|++ \| \| + + \| \| \| | |
| Sbjct: | 8 | TVTDVTSTSLTLSWSPPPDGNGPITGYEVEYQPVNSGEEWNEITVPGTTTSYTLTGLKPG | 67 |
| NOV1: | 1203 | TLYSFFAAARTRKGLGPSS 1221 | |
| | | \| \| \| \| \|\| \| | |
| Sbjct: | 68 | TEYEVRVQAVNGGGNGPPS 86 | |

TABLE 2S

Domain Analysis of NOV2a

gnl|Pfam|pfam00041, fn3, Fibronectin type III domain (SEQ ID NO:96)
CD-Length = 86 residues, 94.2% aligned
Score = 51.2 bits (121), Expect=6e−07

| | | | |
|---|---|---|---|
| NOV 1: | 1235 | PPQNLTLINCTSDFVWLKWSPSPLPGGIVKVYSFK-IHEHETDTIYYKNISGFKTEAKLV | 1293 |
| | | \| \|\|\|+ + \|\| + \| \|\|\| \| \| + \| + + + + \| \| \| | |
| Sbjct: | 3 | APTNLTVTDVTSTSLTLSWSPPPDGNGPITGYEVEYQPVNSGEEWNEITVPGTTTSYTLT | 62 |
| NOV 1: | 1294 | GLEPVSTYSIRVSAFTKVGNG 1314 | |
| | | \|\|+\| + \| +\|\| \| \|\|\| | |
| Sbjct: | 63 | GLKPGTEYEVRVQAVNGGGNG 83 | |

TABLE 2T

Domain Analysis of NOV2a

gnl|Pfam|pfam00041, fn3, Fibronectin type III domain (SEQ ID NO:96)
CD-Length = 86 residues, 100.0% aligned
Score = 49.7 bits (117), Expect = 2e−06

| | | | |
|---|---|---|---|
| NOV1: | 1420 | PSVPTNIAFSDVQSTSATLTWIRPDTILGYFQNYKITTQLRAQKCKEWESEECVEYQKIQ | 1479 |
| | | \|\| \|\|\|+ +\|\| \|\|\| \|\|+\| \| \| \|++ \| \|\| \| | |
| Sbjct: | 1 | PSAPTNLTVTDVTSTSLTLSWSPPPDGNGPITGYEVEYQ------PVNSGEEWNEITV-- | 52 |
| NOV1: | 1480 | YLYEAHLTEETVYGLKKFRWYRFQVAASTNAGYGNAS 1516 | |
| | | \| \|+ \|\|\| \| +\| \| \| \| \| | |
| Sbjct: | 53 | ---PGTTTSYTLTGLKPGTEYEVRVQAVNGGGNGPPS 86 | |

TABLE 2U

Domain Analysis of NOV2a

gnl|Pfam|pfam00041, fn3, Fibronectin type III domain (SEQ ID NO:96)
CD-Length = 86 residues, 98.8% aligned
Score = 47.4 bits (111), Expect = 9e−06

| | | | |
|---|---|---|---|
| NOV 1: | 940 | DPPKDVYYANLSSSSIILFWTPPSKPNGIIQYYSVYYRNT-SGTFMQNFTLHEVTNDFDN | 998 |
| | | \| ++ +++ +\|+ \|\|+\|\| \|\| \| \| \| \|+ \|\| \|+ \| | |
| Sbjct: | 2 | SAPTNLTTDVTSTSLTLSWSPPPDGNGPITGYEVEYQPVNSGEEWNEITVPGTTT---- | 57 |
| NOV 1: | 999 | MTVSTIIDKLTIFSYYTFWLTASTSVGNGNKS 1030 | |
| | | \| + \| + \| + \| \|\|\| \| | |
| Sbjct: | 58 | ---SYTLTGLKPGTEYEVRVQAVNGGGNGPPS 86 | |

TABLE 2V

Domain Analysis of NOV2a

gnl|Pfam|pfam00041, fn3, Fibronectin type III domain (SEQ ID NO:96)
CD-Length 86 residues, 91.9% aligned
Score = 47.0 bits (110), Expect = 1e−05

| | | | |
|---|---|---|---|
| NOV1: | 1530 | GPPENVHVVATSPFSISISWSEPAVITGP-TCYLIDVKSVDNDEFNISFIKSNEENKTIE | 1588 |
| | | \| \|+ \| + \|+++\|\|\| \| \|\| \| \| ++ + \|++ \| + | |
| Sbjct: | 2 | SAPTNLTVTDVTSTSLTLSWSPPPDGNGPITGYEVEYQPVNSGEEWNEITVPGTTT-SYT | 60 |
| NOV1: | 1589 | IKDLEIFTRYSVVITAFTGN 1608 | |
| | | + \|+ \| \| \| + \| \| | |
| Sbjct: | 61 | LTGLKPGTEYEVRVQAVNGG 80 | |

TABLE 2W

Domain Analysis of NOV2a

gnl|Pfam|pfam00041, fn3, Fibronectin type III domain (SEQ ID NO:96)
CD-Length = 86 residues, 96.5% aligned
Score = 46.6 bits (109), Expect = 2e−05

| | | | |
|---|---|---|---|
| NOV1: | 1633 | DPPNNMTFQKIPDEVTKFQLTFLPPSQPNGNIQVYQALVYREDDPTAVQIHNLSIIQKTN | 1692 |
| | | \| \|+\| + \| \|++ \|\| \|\| \| \|+ + + + | |
| SbjCt: | 2 | SAPTNLTVTDVTS--TSLTLSWSPPPDGNGPITGYEVEYQPVNSGEEWNEITVPGTTTS- | 58 |
| NOV1: | 1693 | TFVIAMLEGLKGGHTYNISVYAVNSAGAGP 1722 | |
| | | \| \|\|\| \| \| + \| \|\|\| \| \|\| | |
| Sbjct: | 59 | ----YTLTGLKPGTEYEVRVQAVNGGGNGP 84 | |

TABLE 2X

Domain Analysis of NOV2a

gnl|Pfam|pfam00041, fn3, Fibronectin type III domain (SEQ ID NO:96)
CD-Length = 86 residues, 98.8% aligned
Score = 44.7 bits (104), Expect = 6e−05

| | | | |
|---|---|---|---|
| NOV 1: | 303 | GPPQNCVTGNITGKSFSILWDPPTIVTGKFS-YRVELY---GPSAGRILDNSTKDLKFAF | 358 |
| | | \| \| ++\| \| ++ \| \|\| \| + \| \|\| + + | |
| Sbjct: | 2 | SAPTNLTVTDVTSTSLTLSWSPPPDGNGPITGYEVEYQPVNSGEEWNEITVPGTTTSYTL | 61 |
| NOV 1: | 359 | TNLTPFTMYDVYIAAETSAGTGPKS 383 | |
| | | \| \| \| \| \|+\| + \| \| \|\| \| | |
| Sbjct: | 62 | TGLKPGTEYEVRVQAVNGGGNGPPS 86 | |

TABLE 2Y

Domain Analysis of NOV2a

gnl|Pfam|pfam0004l, fn3, Fibronectin type III domain (SEQ ID NO:96)
CD−Length 86 residues, 100.0% aligned
Score 43.1 bits (100), Expect=2e−04

| | | | |
|---|---|---|---|
| NOV 1: | 561 | PLSAQNGRVTHVTITEVFLHWDPPDPVF--FHHYLITILDVENQSKSIILRTLNSLSLVL | 618 |
| | | \| + \| \|\| \|\| \| + \| \| \|\| \| + \| + + + + + | |
| Sbjct: | 1 | PSAPTNLTVTDVTSTSLTLSWSPPPDGNGPITGYEVEYQPVNSGEEWNEITVPGTTTSYT | 60 |
| NOV 1: | 619 | I-GLKKYTKYKMRVAASTHVGESSLS 643 | |
| | | + \|\|\| \|+\|++\|\| \| \| \| | |
| Sbjct: | 61 | LTGLKPGTEYEVRVQAVNGGGNGPPS 86 | |

TABLE 2Z
Domain Analysis of NOV2a

gnl|Pfam|pfam00041, fn3, Fibronectin type III domain (SEQ ID NO:96)
CD−Length=86 residues, 93.0% aligned
Score=38.5 bits (88), Expect=0.004

| | | | |
|---|---|---|---|
| NOV1: | 1047 | VGNLTYESISSTAINVSWVPPAQPNGLVFYY-VSLILQQTPRHVRPPLVT-YERSIYFDN | 1104 |
| | | \|\|\| ++\|\|++ +\|\| \|\| \|\| + \| \| + \| \| | |
| Sbjct: | 4 | PTNLTVTDVTSTSLTLSWSPPPDGNGPITGYEVEYQPVNSGEWNEITVPGTTTSYTLTG | 63 |
| NOV1: | 1105 | LEKYTDYILKITPSTEKGFS 1124 | |
| Sbjct: | 64 | LKPGTEYEVRVQAVNGGGNG 83 | |

TABLE 2AA

Domain Analysis of NOV2a

gnl|Smart|smart00060, FN3, Eibronectin type 3 domain; One of three
types of internal repeat within the plasma protein, fibronectin. The
tenth fibronectin type III repeat contains a RGD cell recognition
sequence in a flexible loop between 2 strands. Type III modules are
present in both extracellular and intracellular proteins. (SEQ ID NO:97)
CD−Length=83 residues, 96.4% aligned
Score=54.7 bits (130), Expect=6e−08

| | | | |
|---|---|---|---|
| NOV 1: | 54 | PGPPVFLAGERVGSAGILLSWNTPPNP-NGRIISYIVKYKEVCPWMQTVYTQVRSKPDSL | 112 |
| | | \| \|\| \| \| \| + \|\|\| \|\|+ \| \|+ \| \|+\|+\| \| + \| + | |
| Sbjct: | 1 | PSPPSNLRVTDVTSTSVTLSWEPPPDDITGYIVGYRVEYREEGEWKEVNVTP----SSTT | 56 |
| NOV 1: | 113 | EVLLTNLNPGTTYEIKVAAENSAG 136 | |
| | | \|\| \| \|\|\| \|\| +\| \| \| | |
| Sbjct: | 57 | SYTLTGLKPGTEYEFRVRAVNGEA 80 | |

TABLE 2BB

Domain Analysis of NOV2a

gnl|Smart|smart00060, FN3, Fibronectin type 3 domain; One of three
types of internal repeat within the plasma protein, fibronectin. The
tenth fibronectin type III repeat contains a RGD cell recognition
sequence in a flexible loop between 2 strands. Type III modules are
present in both extracellular and intracellular proteins. (SEQ ID NO:97:)
CD−Length=83 residues, 92.8% aligned
Score=52.8 bits (125), Expect=2e−07

| | | | |
|---|---|---|---|
| NOV 1: | 659 | SSPQDVEVIDVTADEIRLKWSPPEKP-NGIIIAYEVLYKNID---TLYMKNTSTTDIILR | 714 |
| | | \| \| ++ \| \|\|\|+ + \| \| \|\| \| \|+ \| \| \|+ + + +\|\|\|+++\| | |
| Sbjct: | 2 | SPPSNLRVTDVTSTSVTLSWEPPPDDITGYIVGYRVEYREEGEWKEVNVTPSSTTSYTLT | 61 |
| NOV 1: | 715 | NLRPHTLYNISVRSYTR 731 | |
| | | \|+\| \| \| \|\|+ | |
| Sbjct: | 62 | GLKPGTEYEFRVRAVNG 78 | |

TABLE 2CC
Domain Analysis of NOV2a

gnl|Smart|smart00060, FN3, Fibronectin type 3 domain; One of three
types of internal repeat within the plasma protein, fibronectin. The
tenth fibronectin type III repeat contains a RGD cell recognition
sequence in a flexible loop between 2 strands. Type III modules are
present in both extracellular and intracellular proteins. (SEQ ID NO:97)
CD−Length=83 residues, 94.0% aligned
Score=45.4 bits (106), Expect=3e−05

| | | | |
|---|---|---|---|
| NOV1: | 1235 | PPQNLTLINCTSDFVWLKWSPSPLPGGIVKVYSFKIHEHETDTIYYKNISGFKTEAKLVG | 1294 |
| | | \|\| \|\| + + \|\| \| \| \| \| \| \| + \| + \| \| \| | |
| Sbjct: | 3 | PPSNLRVTDVTSTSVTLSWEPPPDDITGYIVGYRVEYREEGEWKEVNVTPSSTTSYTTG | 62 |
| NOV 1: | 1295 | LEPVSTYSIRVSAFTKVG 1312 | |
| | | \|+\| + \| \|\| \| | |
| Sbjct: | 63 | LKPGTEYEFRVRAVNGEA 80 | |

TABLE 2DD

Domain Analysis of NOV2a

gnl|Smart|smart00060, FN3, Fibronectin type 3 domain; One of three
types of internal repeat within the plasma protein, fibronectin. The
tenth fibronectin type III repeat contains a RGD cell recognition
sequence in a flexible loop between 2 strands. Type III nodules are
present in both extracellular and intracellular proteins. (SEQ ID NO:97)
CD−Length=83 residues, 100.0% aligned
Score=42.7 bits (99), Expect=2e−04

| | | | |
|---|---|---|---|
| NOV 1: | 561 | PLSAQNFRVTHVTITEVFLHWDPPDPVFFHHYLITILDVENQSKSIILRTLNS--LSLVL | 618 |
| | | \| \| \|\|\| \|\| \| \| \| \|+\|\| + + ++ + + + \| \| \| | |
| Sbjct: | 1 | PSPPSNLRVTDVTSTSVTLSWEPPPDDITGYIVGYRVEYREEGEWKEVNVTPSSTTSYTL | 60 |
| NOV 1: | 619 | IGLKKYTKYKMRVAASTHVGESS 641 | |
| | | \|\|\| \|+\|+ \|\| \| | |
| Sbjct: | 61 | TGLKPGTEYEFRVRAVNGEAGEG 83 | |

TABLE 2EE
Domain Analysis of NOV2a

gnl|Smart|smart00060, FN3, Fibronectin type 3 domain; One of three
types of internal repeat within the plasma protein, fibronectin. The
tenth fibronectin type III repeat contains a RGD cell recognition
sequence in a flexible loop between 2 strands. Type III modules are
present in both extracellular and intracellular proteins. (SEQ ID NO:97)
CD-Length = 83 residues, 92.8% aligned
Score = 41.2 bits (95), Expect = 7e−04

| | | | |
|---|---|---|---|
| NOV1: | 848 | SPPQDFSVKQLSGVTVKLSWQPPLEP-NGIILYYTVYVWRSS----LKTINVTETSLELS | 902 |
| | | \|\|\| + \| ++ +\| \|\|\|+\|\| + \| \|+ \| \| + + \|\| \|+ | |
| Sbjct: | 2 | SPPSNLRVTDVTSTSVTLSWEPPPDDITGYIVGYRVEYREEGEWKEVNVTPSSTTSYTLT | 61 |
| NOV 1: | 903 | DLDYNVEYSAYVTASTR 919 | |
| | | \| \|\| \| \| | |
| Sbjct: | 62 | GLKPGTEYEFRVRAVNG 78 | |

Receptor tyrosine phosphatases (rPTPs) are part of the signaling cascades that control cell survival, proliferation and differentiation. The novel protein tyrosine phosphatase described in the application contains a phosphatase domain and thirteen fibronectin type III repeats. It closely resembles rPTP-GMC1, a rat membrane phosphatase that is expressed in kidney glomerulus and is upregulated in response to kidney injury (Wright et.al. J Biol Chem September 1998 11;273(37):23929-37). Tissue specificity of PTPs varies widely; for eg rPTP-GMC1 is expressed by mesangial cells in the kidney while GLEPP1 (another membrane phosphatase) is expressed by podocytes in the kidney ( Thomas et. al. ; J Biol Chem August 1998 5;269(31):19953-62). Tappia et. al. demonstrated expression of a PTP in the liver could regulate the activity of the insulin and EGF receptors (Tappia et. al.; Biochem J May 1993 15;292 (Pt 1): 1-5).A number of phosphatases have been demonstrated to play a role in cancer, for eg. PTP zeta; a membrane phosphatase; is expressed in brain and is also expressed by a glioblastoma cell line (Krueger et. al.; Proc Natl Acad Sci USA August 1992 15;89(16):7417-21); rPTP alpha is expressed in breast tumors and correlates with tumor grade (Ardini et. al.; Oncogene October 2000 12;19(43):4979-87). This phosphatase (rPTP alpha) is also expressed by human prostate cancer cell lines, oral squamous cell carcinoma and was correlated with histological grade of the oral tumor (Zelivianski et. al.; Mol Cell Biochem May 2000 ;208(1-2):11-8; Berndt et al.; Histochem Cell Biol May 1999 ;111(5):399-403). PTP-1B has been suggested to play arole in diabetes and obesity ( Kennedy et. al.; Biochem Pharmacol October 2000 1;60(7):877-83) whle mutations in a PTP named EPM2A have been suggested as the cause of Lafora's disease ( and autosomal recessive form of progressive myoclonus epilepsy) (Minassian et. al. Nat Genet October 1998 ;20(2):171-4). Given the wide ranging effects of this family of proteins, we hypothesize that the novel protein described in this application plays a role in cancer, neurological, immune and metabolic diseases.

The disclosed NOV2 nucleic acid of the invention encoding a Protein tyrosine phosphatase precursor-like protein includes the nucleic acid whose sequence is provided in Table 2A, 2C, or 2E or a fragment thereof. The invention also includes a mutant or variant nucleic acid any of whose bases may be changed from the corresponding base shown in Table 2A, 2C, or 2E while still encoding a protein that maintains its Protein tyrosine phosphatase precursor like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 16 percent of the bases may be so changed.

The disclosed NOV2 protein of the invention includes the Protein tyrosine phosphatase precursor-like protein whose sequence is provided in Table 2B, 2D, or 2F. The invention also includes a mutant or variant protein any of whose residues may be changed from the corresponding residue shown in Table 2B, 2D, or 2F while still encoding a protein that maintains its Protein tyrosine phosphatase precursor-like activities and physiological functions, or a functional fragment thereof. In the mutant or variant protein, up to about 18 percent of the residues may be so changed.

The invention further encompasses antibodies and antibody fragments, such as F_{ab} or (F_{ab})₂, that bind immunospecifically to any of the proteins of the invention.

The above defined information for this invention suggests that this Protein tyrosine phosphatase precursor-like protein (NOV2) may function as a member of a “Protein tyrosine phosphatase precursor family”. Therefore, the NOV2 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV2 nucleic acids and proteins of the invention are useful in potential therapeutic applications implicated in cancer including but not limited to various pathologies and disorders as indicated below. For example, a cDNA encoding the Protein tyrosine phosphatase precursor-like protein (NOV2) may be useful in gene therapy, and the Protein tyrosine phosphatase precursor-like protein (NOV2) may be useful when administered to a subject in need thereof. By way of nonlimiting example, the compositions of the present invention will have efficacy for treatment of patients suffering from cancer, kidney cancer, trauma, regeneration (in vitro and in vivo), viral/bacterial/parasitic infections, nephrological disesases including diabetes, autoimmune disease, renal artery stenosis, interstitial nephritis, glomerulonephritis, polycystic kidney disease, systemic lupus erythematosus, renal tubular acidosis, IgA nephropathy, hypercalceimia, Lesch-Nyhan syndrome, Hirschsprung's disease, Crohn's Disease, appendicitis, or other pathologies or conditions. The NOV2 nucleic acid encoding the Protein tyrosine phosphatase precursor-like protein of the invention, or fragments thereof, may further be useful in diagnostic applications, wherein the presence or amount of the nucleic acid or the protein are to be assessed.

NOV2 nucleic acids and polypeptides are further useful in the generation of antibodies that bind immuno-specifically to the novel NOV2 substances for use in therapeutic or diagnostic methods. These antibodies may be generated according to methods known in the art, using prediction from hydrophobicity charts, as described in the “Anti-NOVX Antibodies” section below. The disclosed NOV2 proteins have multiple hydrophilic regions, each of which can be used as an immunogen. In one embodiment, a contemplated NOV2 epitope is from about amino acids 1 to 100. In another embodiment, a NOV2 epitope is from about amino acids 200 to 300. In further embodiments, a NOV2 epitope is from about amino acids 450 to 500, from about amino acids 600 to 900, from about amino acids 950 to 1000, from about amino acids 1200 to 1300, from about amino acids 1400 to 1600, from about amino acids 1800 to 1900, from about amino acids 1950 to 2050, and from about amino acids 2200 to 2300. These novel proteins can be used in assay systems for functional analysis of various human disorders, which will help in understanding of pathology of the disease and development of new drug targets for various disorders.

NOV3

A disclosed NOV3 nucleic acid of 4538 nucleotides (also referred to as 134899552_EXT) encoding a novel human homolog of the Drosophila pecanex-like protein is shown in Table 3A. An open reading frame was identified beginning with an ATG initiation codon at nucleotides 101-103 and ending with a TGA codon at nucleotides 4439-4441. A putative untranslated region upstream from the initiation codon and downstream from the termination codon is underlined in Table 3A, and the start and stop codons are in bold letters.

TABLE 3A
NOV3 nucleotide sequence.

(SEQ ID NO:11)

CATGAAGGAAAAATTCTGAGTATTCTAATGGCTTTTTAAAATAATCATTTATTTGCTAGGTAAGTTCTCTTC
TACGCTGTATGAGACTGGTGGCTGTGATATGTCACTTGTGAATTTTGAACCAGCAGCAAGAAGAGCATCCAA
TATCTGGGACACAGATTCTCATGTATCCAGTTCTACCTCAGTTCGATTTTATCCACATGATGTGATTCGATT
GAATAGACTATTGACCATTGATACAGATTTGTTGGAGCAACAGGACATTGATCTAAGCCCTGACTTGGCAGC
TACTTACGGCCCAACAGAAGAAGCTGCCCAAAAGGTTAAACACTATTATCGCTTTTGGATCCTACCCCAGCT
GTGGATTGGCATTAACTTTGACAGACTCACACTTTGGCCCTGTTTGATAGGAATCGTGAGATCCTGGAAAA
TGTGTTAGCTGTCATCCTGGCTATTCTCGTGGCCTTTTTGGGATCTATTCTTCTCATACAAGGATTCTTCAG
AGATATCTGGGTCTTCCAGTTCTGCCTCGTCATAGCCAGCTGTCAATACTCACTGCTTAAGAGTGTTCAACC
AGATTCTTCTTCTCCCAGACATGGTCATAATCGTATCATTGCCTACAGTAGACCAGTTTATTTCTGCATATG
TTGCGGTCTTATTTGGCTCTTGGATTATGGTAGCAGAAACCTGACTGCAACCAAGTTCAAATTATATGGAAT
AACTTTCACCAATCCACTGGTGTTTATATCAGCCAGGGATTTAGTTATAGTGTTTACACTCTGTTTCCCAAT
AGTGTTTTTCATTGGTCTCCTGCCTCAGGTGAATACATTTGTAATGTACCTTTGTGAACAATTGGATATTCA
TATTTTTGGTGGTAATGCCACTACAAGCCTGCTTGCAGCACTTTACAGTTTTATCTGTAGCATTGTTGCAGT
AGCCTTATTGTATGGATTATGTTATGGGGCTTTACAGGATTCTTGGGATGGCCAGCATATTCCAGTACTTTT
CTCCATTTTTTGTGGTTTATTAGTGGCAGTGTCTTACCATCTCAGCCGACAAAGCAGTGATCCATCTGTACT
TAGCTCTTTAGTGCAATCCAAGATTTTTCCAAAAACGGAAGAGAAAAATCCAGAAGACCCTCTATCTGAAGT
AAAAGATCCACTGCCTGAAAAACTTAGAAATTCTGTTAGTGAGCGATTACAGTCTGACCTGGTAGTATGCAT
TGTAATTGGTGTGCTGTATTTTGCTATTCATGTAAGCACAGTCTTCACAGTATTGCAGCCTGCCCTCAAGTA
TGTGTTGTATACATTGGTTGGCTTTGTGGGTTTTGTAACCCATTATGTGCTGCCTCAAGTTAGAAAACAGCT
ACCATGGCACTGTTTCTCTCATCCTCTGCTAAAGACACTAGAGTATAATCAGTATGAAGTTCGAGATGCAGC
CACTATGATGTGGTTTGAGAAACTTCATGTGTGGCTTCTTTTTGTGGAGAAGAATATAATCTATCCATTGAT
TGTTCTCAATGAACTGAGCAGCAGTGCAGAGACAATTGCTAGTCCAAAGAAACTGAATACAGAGTTAGGTGC
TTTAATGATCACTGTTGCTGGTTTGAAGTTGCTACGATCCTCTTTTAGCAGCCCTACATATCAGTATGTTAC
AGTCATCTTTACTGTGCTGTTTTTCAAATTTGACTATGAAGCTTTTTCAGAGACCATGCTGTTGGATCTCTT
CTTTATGTCCATACTCTTCAACAAGCTTTGGGAACTACTTTATAAATTGCAGTTTGTGTATACCTATATTGC
CCCATGGCAGATCACATGGGGTTCTGCTTTCCATGCTTTTGCTCAGCCTTTTGCAGTGCCTCGTTCAGCCAT
GCTGTTTATTCAGGCTGCTGTCTCGGCCTTCTTCTCTACTCCACTGAACCCCTTTCTGGGAAGTGCAATATT
CATCACTTCATATGTCCGACCTGTGAAATTCTGGGAGAGAGACTATAGCACAAAACGAGTGGATCATTCAAA
TACCAGATTGGCTTCCCAGCTTGATAGAAATCCAGGTTCAGATGACAACAATCTGAATTCCATCTTTTATGA
GCATTTAACTAGATCCCTACAGCACAGCCTCTGTGGTGATTTGCTACTAGGACGGTGGGGAAACTACAGTAC
AGGGGACTGTTTCATCCTTGCCTCTGACTATCTCAATGCATTAGTACACCTTATAGAGATAGGCAATGGTCT
GGTCACTTTTCAGCTGCGGGGACTTGAATTCAGAGGTACCTACTGTCAACAACGGGAAGTGGAGGCCATTAC
TGAAGGTGTAGAGGAAGATGAAGGATTTTGCTGTTGTGAACCTGGCCATATTCCTCACATGCTTTCATTTAA
TGCTGCATTTAGCCAGCGATGGCTAGCTTGGGAAGTGATAGTCACAAAGTACATTCTGGAGGGTTATAGCAT
CACTGATAACAGTGCTGCTTCTATGCTTCAAGTCTTTGATCTTCGGAAAGTACTCACCACTTACTATGTCAA
GGGTATCATTTATTATGTTACGACCTCGTCTAAGCTAGAGGAGTGGCTAGCTAATGAGACAATGCAGGAAGG
ACTTCGTCTGTGTGCTGATCGCAATTATGTCGATGTGGACCCGACCTTTAATCCAAACATTGATGAAGACTA
TGACCACCGACTGGCAGGCATATCTAGGGAGAGTTTCTGTGTGATTTACCTCAACTGGATAGAGTACTGCTC
TTCCCGAAGAGCAAAGCCTGTGGATGTGGACAAAGATTCATCCCTAGTGACTCTCTGTTATGGACTCTGTGT
TCTGGGACGGAGAGCTTTGGGGACTGCATCCCATCATATGTCCAGTAATTTAGAGTCATTCCTCTATGGATT
GCATGCCCTATTTAAAGGAGATTTCCGTATTTCTTCAATTCGAGATGAATGGATCTTTGCTGACATGGAATT
GCTAAGAAAAGTAGTAGTCCCTGGGATCCGTATGTCCATTAAACTTCATCAGGATCATTTTACTTCTCCAGA
TGAATATGATGACCCTACTGTGCTCTATGAAGCCATAGTATCTCATGAGAAGAACCTCGTAATAGCCCATGA
AGGGGACCCTGCATGGCGGAGTGCAGTACTTGCCAACTCTCCCTCCTTGCTTGCTCTGCGGCATGTCATGGA
TGATGGCACCAATGAATATAAAATCATCATGCTCAACAGACGCTACCTGAGCTTCAGGGTCATTAAAGTGAA
TAAGGAATGTGTCCGAGGTCTTTGGGCAGGGCAACAGCAGGAGCTTGTTTTTCTACGTAACCGTAACCCAGA
TAGAGGTAGCATCCAAAATGCAAAGCAAGCCCTGAGAAACATGATAAACTCATCTTGTGATCAACCTATTGG
CTACCCAATCTTTGTCTCACCCCTGACAACTTCTTACTCTGACAGCCACGAACAGCTTAAAGACATTCTTGG
GGGTCCTATCAGCTTGGGAAATATCAGGAACTTCATAGTGTCAACCTGGCACAGGCTTAGGAAAGGTTGCGG
AGCTGGATGTAACAGTGGTGGCAATATTGAAGATTCTGATACTGGAGGTGGGACTTCCTGCACTGGTAACAA
TGCAACAACTGCCAACAATCCCCACAGCAACGTGACCCAGGGAAGCATTGGAAATCCTGGGCAGGGATCAGG
AACTGGACTCCACCCACCTGTCACATCTTATCCTCCAACACTAGGTACTAGCCACAGCTCTCACTCTGTGCA
GTCGGGCCTGGTCAGACAGTCTCCTGCCCGGGCCTCAGTAGCCAGCCAGTCTTCCTACTGCTATAGCAGCCG
GCATTCATCCCTCCGGATGTCCACCACTGGGTTTGTGCCTTGTCGGCGCTCTTCTACTAGTCAGATATCGCT
TCGAAACTTGCCATCATCCATCCAATCCCGACTGTCGATGGTGAACCAAATGGAACCCTCAGGTCAGAGCGG
CCTGGCCTGTGTGCAGCACGGCCTGCCTTCCTCCAGCAGCTCCAGCCAAAGCATCCCAGCCTGCAAACATCA
CACTCTCGTGGGCTTTCTTGCGACAGACCCAGGTCAGAGCAGTGCCACTGATGCACAGCCAGGCAACACCTT
AAGTCCTGCCAACAATTCACACTCCAGAAAGGCAGAAGTGATTTACAGAGTCCAAATTGTGGATCCCAGTCA
AATTCTGGAAGGGATCAACCTGTCTAAAAGGAAAGAGCTACAGTGGCCTGATGAAGGAATCCGGTTAAAAGC
TGGGAGAAATAGCTGGAAAGACTGGAGTCCGCAGGAGGGCATGGAAGGCCATGTGATTCACCGATGGGTGCC
TTGCAGCAGAGATCCAGGTACCAGATCCCACATCGACAAGGCAGTGCTTCTGGTCCAGATTGATGATAAATA
TGTGACTGTAATTGAAACTGGGGTACTAGAACTTGGGGCTGAAGTGTGAGCCAGTGTTTATTATAAAGACAT
TTCTTTTTCCCTCTCAATTCCAAGGCATTGGAAAAAGAGAGGAACAAGCAGAAGATGCCTGCAGGTATCACT
TT

The disclosed NOV3 nucleic acid sequence, localized to chromsome 14, has 2277 of 2283 bases (99%) identical to a gb:GENBANK-ID:AB018348|acc:AB018348.1 mRNA from Homo sapiens (Homo sapiens mRNA for KIAA0805 protein, partial cds) (E=0.0).

A NOV3 polypeptide (SEQ ID NO: 12) encoded by SEQ ID NO: 11 has 1446 amino acid residues and is presented using the one-letter code in Table 3B. Signal P, Psort and/or Hydropathy results predict that NOV3 does not contain a signal peptide and is likely to be localized to the plasma membrane with a certainty of 0.8000. In other embodiments, NOV3 may also be localized to the mitochondrial inner membrane with a certainty of 0.4714, the Golgi body with a certainty of 0.4000, or the endoplasmic reticulum (membrane) with a certainty of 0.3000.

TABLE 3B
Encoded NOV3 protein sequence.

(SEQ ID NO:12)

MSLVNFEPAARRASNIWDTDSHVSSSTSVRFYPHDVIRLNRLLTIDTDLLEQQDIDLSPDLAATYGPTEEAA
QKVKHYYRFWILPQLWIGINFDRLTLLALFDRNREILENVLAVILAILVAFLGSILLIQGFFRDIWVFQFCL
VIASCQYSLLKSVQPDSSSPRHGHNRIIAYSRPVYFCICCGLIWLLDYGSRNLTATKFKLYGITFTNPLVFI
SARDLVIVFTLCFPIVFFIGLLPQVNTFVMYLCEQLDIHIFGGNATTSLLAALYSFICSIVAVALLYGLCYG
ALQDSWDGQHIPVLFSIFCGLLVAVSYHLSRQSSDPSVLSSLVQSKIFPKTEEKNPEDPLSEVKDPLPEKLR
NSVSERLQSDLVVCIVIGVLYFAIHVSTVFTVLQPALKYVLYTLVGFVGFVTHYVLPQVRKQLPWHCFSHPL
LKTLEYNQYEVRDAATMMWFEKLHVWLLFVEKNIITPLIVLNELSSSAETIASPKKLNTELGALMITVAGLK
LLRSSFSSPTYQYVTVIFTVLFFKFDYEAFSETMLLDLFFMSILFNKLWELLYKLQFVYTYIAPWQITWGSA
FHAFAQFPAVPRSAMLFIQAAVSAFFSTPLNPFLGSAIFITSYVRPVKFWERDYSTKRVDHSNTRLASQLDR
NPGSDDNNLNSIFYEHLTRSLQHSLCGDLLLGRWGNYSTGDCFILASDYLNALVHLIEIGNGLVTFQLRGLE
FRGTYCQQREVEAITEGVEEDEGFCCCEPGHIPHMLSFNAAFSQRWLAWEVIVTKYILEGYSITDNSAASML
QVFDLRKVLTTYYVKGIIYYVTTSSKLEEWLANETMQEGLRLCADRNYVDVDPTFNPNIDEDYDHRLAGISR
ESFCVIYLNWIEYCSSRRAKPVDVDKDSSLVTLCYGLCVLGRRALGTASHHMSSNLESFLYGLHALFKGDFR
ISSIEDEWIFADMELLRKVVVPGIRMSIKLHQDHFTSPDEYDDPTVLYEAIVSHEKNLVIAHEGDPAWRSAV
LANSPSLLALRHVMDDGTNEYKIIMLNRRYLSFRVIKVNKECVRGLWAGQQQELVFLRNRNPERGSIQNAKQ
ALRNMINSSCDQPIGYPIFVSPLTTSYSDSHEQLKDILGGPISLGNIRNFIVSTWHRLRKGCGAGCNSGGNI
EDSDTGGGTSCTGNNATTANNPHSNVTQGSIGNPGQGSGTGLHPPVTSYPPTLGTSHSSHSVQSGLVRQSPA
RASVASQSSYCYSSRHSSLRMSTTGFVPCRRSSTSQISLRNLPSSIQSRLSMVNQMEPSGQSGLACVQHGLP
SSSSSSQSIPACKHHTLVGFLATEGGQSSATDAQPGNTLSPANNSHSRKAEVIYRVQIVDPSQILEGINLSK
RRELQWPDEGIRLKAGRNSWKDWSPQEGMEGHVIHRWVPCSRDPGTRSHIDKAVLLVQIDDKYVTVIETGVL
ELGAEV

The disclosed NOV3 amino acid sequence has 1355 of 1446 amino acid residues (93%) identical to, and 1409 of 1446 amino acid residues (97%) similar to, the 1446 amino acid residue ptnr:SPTREMBL-ACC:Q9QYC1 protein from Mus musculus (Mouse) (PECANEX 1) (E=0.0).

NOV3 is expressed in at least Pancreas, Parathyroid Gland, Thyroid, Mammary gland/Breast, Ovary, Placenta, Uterus, Colon, Liver, Bone Marrow, Lymphoid tissue, Spleen, Tonsils, Prostate, Testis, Brain, Lung, and Kidney. This information was derived by determining the tissue sources of the sequences that were included in the invention including but not limited to SeqCalling sources, Public EST sources, Literature sources, and/or RACE sources.

In addition, NOV3 is predicted to be expressed in Homo sapiens heart, melanocyte, B-cells, larynx, skin, CNS, and multiple sclerosis lesions because of the expression pattern of the following sequences (which are publicly availabel ESTS for the sequence of the invention) AB018348, BE881203, BE867469, BE867415, AB007895, NM_{—}014801, U74315, BE880986, W500099, AW250617, AA426168, AW246742, AA284182, W46420, H14491, Z44921, BE930588, AI922381, AI215559, AA923742, AA582883, BE797814, N75143, BE049421, F07632, BE797239, AI168579, AV653955, BE065657, AL079849, and BE767656, closely related Homo sapiens mRNA for KIAA proteins, partial cds homolog.

NOV3 also has homology to the amino acid sequences shown in the BLASTP data listed in Table 3C.

TABLE 3C
BLAST results for NOV3

| Gene Index/ | Protein/ | Length | Identity | Positives | |
|---|---|---|---|---|---|
| Identifier | Organism | (aa) | (%) | (%) | Expect |
| ref\|XP_027243.1\| | hypothetical | 619 | 619/619 | 619/619 | 0.0 |
| (XM_027243) | protein | | (100%) | (100%) | |
| | XP_027243 | | | | |
| | [Homo | | | | |
| | sapiens] | | | | |
| gi\|15076843\|gb\|AAK82958.1\| | pecanex-like | 2341 | 1372/1451 | 1376/1451 | 0.0 |
| AF233450_1 | protein 1 | | (94%) | (94%) | |
| (AF233450) | [Homo | | | | |
| | sapiens] | | | | |
| gi\|6650377\|gb\|AAF21809.1\| | pecanex 1 | 1446 | 1296/1446 | 1344/1446 | 0.0 |
| AF096286_1 | [Mus | | (89%) | (92%) | |
| (AF096286) | musculus] | | | | |
| gi\|13171105\|gb\|AAK13590.1\| | pecanex | 1703 | 1079/1466 | 1204/1466 | 0.0 |
| AF154413_1 | [Takifugu | | (73%) | (81%) | |
| (AF154413) | rubripes] | | | | |
| gi\|7290294\|gb\|AAF45755.1\| | pcx gene | 3437 | 320/554 | 424/554 | 0.0 |
| (AE003423) | product [alt | | (57%) | (75%) | |
| | 1] | | | | |
| | [Drosophila | | | | |
| | melanogaster] | | | | |

The homology of these sequences is shown graphically in the ClustalW analysis shown in Table 3D.

[image]

[image]

[image]

[image]

[image]

[image]

Pecanex gene was originally discovered in Drosophila, encoding a large, membrane-spanning protein. The mouse homolog was recently reported. In the absence of maternal expression of the pecanex gene, the embryo develops severe hyperneuralization similar to that characteristic of Notch mutant embryos. Early gastrula embryos, lacking both maternally and zygotically expressed activity of the neurogenic pecanex locus, are shown to contain a greater than wild-type number of stably determined neural precursor cells which can differentiate into neurons in culture. Therefore it is anticipated that this novel human pecanex will be involved in neuronal differentiation, maintenance of neuronal precursors and neurological diseases.

The disclosed NOV3 nucleic acid of the invention encoding a Human homolog of the Drosophila pecanex protein includes the nucleic acid whose sequence is provided in Table 3A or a fragment thereof. The invention also includes a mutant or variant nucleic acid any of whose bases may be changed from the corresponding base shown in Table 3A while still encoding a protein that maintains its Human homolog of the Drosophila pecanex activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 1 percent of the bases may be so changed.

The disclosed NOV3 protein of the invention includes the Human homolog of the Drosophila pecanex protein whose sequence is provided in Table 3B. The invention also includes a mutant or variant protein any of whose residues may be changed from the corresponding residue shown in Table 3B while still encoding a protein that maintains its Human homolog of the Drosophila pecanex activities and physiological functions, or a functional fragment thereof. In the mutant or variant protein, up to about 7 percent of the residues may be so changed.

The NOV3 nucleic acids and proteins of the invention are useful in potential therapeutic applications implicated in cancer,trauma, regeneration (in vitro and in vivo), viral/bacterial/parasitic infections, cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, multiple sclerosis, scleroderma, obesity, endometriosis, fertility, hypercoagulation, autoimmume disease, allergies, immunodeficiencies, transplantation, hemophilia, idiopathic thrombocytopenic purpura, graft versus host disease, Von Hippel-Lindau (VHL) syndrome, Alzheimer's disease, stroke, hypercalceimia, Parkinson's disease, Huntington's disease, cerebral palsy, epilepsy, ataxia-telangiectasia, leukodystrophies, behavioral disorders, addiction, anxiety, pain, neuroprotection, systemic lupus erythematosus, asthma, emphysema, ARDS, laryngitis, psoriasis, actinic keratosis, acne, hair growth/loss, allopecia, pigmentation disorders, endocrine disorders, diabetes, renal artery stenosis, interstitial nephritis, glomerulonephritis, polycystic kidney disease, systemic lupus erythematosus, renal tubular acidosis, IgA nephropathy, Lesch-Nyhan syndrome, and a variety of kidney diseases and/or other pathologies and disorders.

NOV3 nucleic acids and polypeptides are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. The disclosed NOV3 protein has multiple hydrophilic regions, each of which can be used as an immunogen. In one embodiment, a contemplated NOV3 epitope is from about amino acids 20 to 50. In another embodiment, a NOV3 epitope is from about amino acids 180 to 200. In additional embodiments, NOV3 epitopes are from about amino acids 360 to 400, from about 450 to 500, from about amino acids 600 to 680, from about amino acids 720 to 780, from about amino acids 800 to 860, from about amino acids 950 to 1000, from about amino acids 1050 to 1100, from about amino acids 1150 to 1320, and from about amino acids 1350 to 1420. These novel proteins can be used in assay systems for functional analysis of various human disorders, which are useful in understanding of pathology of the disease and development of new drug targets for various disorders.

NOV4

A disclosed NOV4 nucleic acid of 1500 nucleotides (also referred to as SC140515441_A) encoding a novel Aurora-related kinase I-like protein is shown in Table 4A. An open reading frame was identified beginning with a ATG initiation codon at nucleotides 182-184 and ending with a TAG codon at nucleotides 1391-1393. The start and stop codons are in bold letters, and the 5′ and 3′ untranslated regions are underlined.

TABLE 4A
NOV4 Nucleotide Sequence

(SEQ ID NO:13)

TCATCTTAAATTTTTTTAGCTGATATAGTTGTAATTTCTTAACCTAGCTCATCTCTAGAGGATATGTAAAA
ACATAAAACACCTCAATTACTTGTGAATTATAGAGGTGTATCAGTTGGTTTAAAAGTGCTTTTATTGGGCT
GAGCTCTTGGAAGACTCAGGTCCTTGGGTCATAGGCATCATGGACCAATCTGAAGAAAACTGCATTTCAGG
GCCCTGTTGAGGCTAAAACTCCAGTTGGAGGTCCAGAACATGTTCTCGTGACTCAGCAATTTCCTTGTCAGA
ATCCATTACCTGCAAATAGTGGCCAGGCTCAGTGGGTCTTGTGTCCTTCAAATTCTTCGCAGCGTGTTCCT
TTGCAAGCACAAAAGCTTGTCTCCAGTCACAAGCCAGTTCAGAATCAGAAGCAGAAGCAATTGCAGGCAAC
CAGTGTACCTCATCCTGCCTCCAGGCCACTGAATAACACCCAAAACAGCAAGCAGTCCCCGCTGTCGGCAC
CTGAAAATAATCCTGAGGAGGAACTGGCATCAAAACAGAAAAATGAAGAATCAAAAAAGAGGCAATGGGCT
TTGGAAGACCTTGAAATTGGTCGCCCTCCGGGTAAAGGAAAGTTTGGTAATGTTTATTTGGCAAGAGAAAA
ACAAAGCAAGTTTATTCTGGCTCTTAGGGTGTTATTTAAAGCTCAGCTGGAGAAAGCAGGAGTGGAGCATC
AACTCAGAAGAGAAGTAGAAATACAGTCCCACCTCCAACATCCTAATATAATCAGACTGTATGGTTATTTC
CATGATGCCACCAGAGTCTACCTAATTCTGGAATATACACCACTTGAAACAGTCAATACAGAACTTCAGAA
ACTTTCAAAGTTTGATGAGCAGAGAACTGCTACTTATATCACAGAATTGGCAAGTGCCCTGTCTTACTGTC
ATTCAAAAACAGTTATTCATAGAGACATTAAGCCAGAGAACTTACTTCTTGGATCAGCTGGAGAGCTTGAA
ATTGCAAATTTTGGGTGGTCAGAACATGCTCCATCTTCCAGGAGGACCACTCTCTGTGGCACCCTGGACTA
CCTGCCCCCCGAAATGATTGAAGGTCGGATGCATGATGAGAAGGTGGATCTCTGGAGCCTTGGAGTTCTTT
GCTGTGAATTTTTAGTTGGGAAGCCTCCTTTTGAGGCAAATACATACCAAGAGACCTACAAAAGAATATCA
CGGGTTGAGTTCACATTCCCTGACTTTGTAACAGAGGGAGCCAGGGACCTCATTTCAAGACTGTTGAAGCA
TGTTCCCAGCCAGAGGCCAATGCTCAGAGAAGTACTTGAATACCCCTGGATCACAGCAAATTCATCAAAAC
CATCAAATTGCCAAAACAAAGAATCAACTAGCAAGTATTCTTAGGAATCGTGCAGGGGGAGAAATCCTTGA
GCCAGGGCTGCTGTATAACCTCTCAGGAACATGCTACCAAAATTTATTTTACCATTGACTGCTGCCCTCAA
TCTAGAACA

The disclosed NOV4 nucleic acid sequence maps to chromosome 1 and has 1152 of 1212 bases (95%) identical to a gb:GENBANK-ID:AF008551|acc:AF008551 mRNA from Homo sapiens (Homo sapiens aurora-related kinase 1 (ARK1) mRNA, complete cds (E=1.8e⁻²⁴³).

A disclosed NOV4 protein (SEQ ID NO: 14) encoded by SEQ ID NO: 13 has 403 amino acid residues, and is presented using the one-letter code in Table 4B. Signal P, Psort and/or Hydropathy results predict that NOV4 does not have a signal peptide, and is likely to be localized to the cytoplasm with a certainty of 0.4500. In other embodiments NOV4 is also likely to be localized microbody (peroxisome) with a certainty of 0.3000, to the mitochondrial membrane space with a certainty of 0.1000, or to the lysosome(lumen) with a certainty of 0.1000.

TABLE 4B
Encoded NOV4 protein sequence.

(SEQ ID NO:14)

MDQSEENCISGPVEAKTPVGGPEHVLVTQQFPCQNPLPANSGQAQWVLCP
SNSSQRVPLQAQKLVSSHKPVQNQKQKQLQATSVPHPASRPLNNTQNSKQ
SPLSAPENNPEEELASKQKNEESKKRQWALEDLEIGRPPGKGKFGNVYLA
REKQSKFILALRVLFKAQLEKAGVEHQLRREVEIQSHLQHPNIIRLYGYF
HDATRVYLILEYTPLETVNTELQKLSKFDEQRTATYITELASALSYCHSK
TVIHRDIKPENLLLGSAGELEIANFGWSEHAPSSRRTTLCGTLDYLPPEM
IEGRMHDEKVDLWSLGVLCCEFLVGKPPFEANTYQETYKRISRVEFTFPD
FVTEGARDLISRLLKHVPSQRPMLREVLEYPWITANSSKPSNCQNKESTS
KYS

The disclosed NOV4 amino acid has 69 of 403 amino acid residues (91%) identical to, and 381 of 403 amino acid residues (94%) similar to, the 403 amino acid residue ptnr:SPTREMBL-ACC:060445 protein from Homo sapiens (Human) (Aurora-Related Kinase 1 (E=1.7e⁻¹⁹⁸).

NOV4 is expressed in at least Adrenal Gland/Suprarenal gland, Amygdala, Bone Marrow, Brain, Cervix, Colon, Coronary Artery, Epidermis, Heart, Kidney, Liver, Lung, Lymphoid tissue, Mammary gland/Breast, Ovary, Peripheral Blood, Placenta, Prostate, Testis, Thalamus, Tonsils, Uterus. This information was derived by determining the tissue sources of the sequences that were included in the invention.

In addition, NOV4 is predicted to be expressed in colon because of the expression pattern of (GENBANK-ID: gb:GENBANK-ID:AF008551|acc:AF008551) a closely related aurora-related kinase 1 (ARK1) mRNA, complete cds homolog in species Homo sapiens.

NOV4 also has homology to the amino acid sequences shown in the BLASTP data listed in Table 4C.

TABLE 4C
BLAST results for NOV4

| Gene Index/ | Protein/ | Length | Identity | Positives | |
|---|---|---|---|---|---|
| Identifier | Organism | (aa) | (%) | (%) | Expect |
| gi\|12654873\|gb\|AAH01280.1\| | serine/threonine | 403 | 370/403 | 381/403 | 0.0 |
| AAH01280 | kinase | | (91%) | (93%) | |
| (BC001280) | 15 [Homo | | | | |
| | sapiens] | | | | |
| gi\|13653970\|ref\|XP_{—} | serine/threonine | 403 | 369/403 | 381/403 | 0.0 |
| 009546.3\| | kinase | | (91%) | (93%) | |
| (XM_009546) | 15 [Homo | | | | |
| | sapiens] | | | | |
| gi\|4507275\|ref\|NP_003591.1\| | serine/threonine | 403 | 369/403 | 380/403 | 0.0 |
| (NM_003600) | kinase | | (91%) | (93%) | |
| | 15; | | | | |
| | Serine/threonine | | | | |
| | protein | | | | |
| | kinase 15 | | | | |
| | [Homo | | | | |
| | sapiens] | | | | |
| gi\|7446411\|pir\|\|JC5974 | aurora- | 403 | 367/403 | 379/403 | 0.0 |
| | related | | (91%) | (93%) | |
| | kinase 1 (EC | | | | |
| | 2.7.-.-) - | | | | |
| | human | | | | |
| gi\|4507279\|ref\|NP_003149.1\| | serine/threonine | 402 | 342/403 | 360/403 | 0.0 |
| (NM_003158) | kinase 6; | | (84%) | (88%) | |
| | Serine/threonine | | | | |
| | protein | | | | |
| | kinase-6; | | | | |
| | serine/threonine | | | | |
| | kinase 6 | | | | |
| | (aurora/IPL1- | | | | |
| | like) [Homo | | | | |
| | sapiens] | | | | |

The homology of these sequences is shown graphically in the ClustalW analysis shown in Table 4D.

[image]

[image]

Tables 4E-G lists the domain description from DOMAIN analysis results against NOV4. This indicates that the NOV4 sequence has properties similar to those of other proteins known to contain this domain.

TABLE 4E
Domain Analysis of NOV4

gnl|Smart|smart00220, S_TKc, Serine/Threonine protein kinases,
catalytic domain; Phosphotransterases. Serine or threonine-specific
kinase subfamily. (SEQ ID NO:98)
CD-Length = 256 residues, 99.6% aligned
Score = 256 bits (653), Expect = 2e−69

| | | | |
|---|---|---|---|
| NOV 3: | 134 | EIGRPPGKGKFGNVYLAREKQSKFILALRVLFKAQLEKAGVEHQLRREVEIQSHLQHPNI | 193 |
| | | \|+ \|\|\| \|\| \|\|\|\|\|+\|++ ++\|++\|+ \| +\|+\| ++ \|\|++\| \| \|\|\|\| | |
| Sbjct: | 2 | ELLEVLGKGAFGKVYLARDKKTGKLVAIKVIKKEKLKK-KKRERILREIKILKKLDHPNI | 60 |
| NOV 3: | 194 | IRLYGYFHDATRVYLILEYTPLETVNTELQKLSKFDEQRTATYITELASALSYCHSKTVI | 253 |
| | | ++\|\| \| \| ++\|\|++\|\| + \|+\| + \| \| ++ \|\|\| \| \|\|+ +\| | |
| Sbjct: | 61 | VKLYDVFEDDDKLYLVMEYCEGGDLFDLLKKRGRLSEDEARFYARQILSALEYLHSQGII | 120 |
| NOV 3: | 254 | HRDIKPENLLLGSAGELEIANFGWS--EHAPSSRRTTLCGTLDYLPPEMIEGRMHDEKVD | 311 |
| | | \|\|\|+\|\|\|\|+\|\| \| \| +++\|+\|\| + + + \|\| \|\| +\|+ \|\|++ \|+ + + \|\| | |
| Sbjct: | 121 | HRDLKPENILLDSDGHVKLADFGLAKQLDSGGTLLTTFVGTPEYMAPEVLLGKGYGKAVD | 180 |
| NOV 3: | 312 | LWSLGVLCCEFLVGKPPFEA-NTYQETYKRISRVEFTFPDF---VTEGARDLISRLLKHV | 367 |
| | | +\|\|\|\|\|+ \| \| \|\|\|\|\| + +\|+\| + \|\| ++ \|+\|\|\| +\|\| | |
| Sbjct: | 181 | IWSLGVILYELLTGKPPFPGDDQLLALFKKIGKPPPPFPPPEWKISPEAKDLIKKLLVKD | 240 |
| NOV 3: | 368 | PSQRPMLREVLEYPWT 383 | |
| | | \| +\| \| \|\|+\|+ | |
| Sbjct: | 241 | PEKRLTAEEALEHPFF 256 | |

TABLE 4F
Domain Analysis of NOV4

gnl|Pfam|pfam00069, pkinase, Protein kinase domain (SEQ ID NO:99)
CD-Length = 256 residues, 100.0% aligned
Score = 221 bits (564), Expect = 5e−59

| | | | |
|---|---|---|---|
| NOV 3: | 133 | LEIGRPPGKGKFGNVYLAREKQSKFILALRVLFKAQLEKAGVEHQLRREVEIQSHLQHPN | 192 |
| | | \|+\| \| \| \|\| \|\| + \| + \|+\|+++\| \| \| + + + \|\|++\| \| \|\|\| | |
| Sbjct: | 1 | YELGEKLGSGAFGKVYKGKHKDTGEIVAIKILKKRSLSE--KKKRFLREIQILRRLSHPN | 58 |
| NOV 3: | 193 | IIRLYGYFHDATRVYLILEYTPLETVNTEL-QKLSKFDEQRTATYITELASALSYCHSKT | 251 |
| | | \|+\|\| \| \| + +\|\|++\|\| + \| + \|+ ++ \| \| \|\|+ | |
| Sbjct: | 59 | IVRLLGVFEEDDHLYLVMEYMEGGDLFDYLRRNGLLLSEKEAKKIALQILRGLEYLHSRG | 118 |
| NOV 3: | 252 | VIHRDIKPENLLLGSAGELEIANFGWS---EHAPSSRRTTTLCGTLDYLPPEMIEGRMHDE | 308 |
| | | ++\|\|\|+\|\|\|\|+\|\| \| ++\|\|+\|\| + \| + + \|\| \|\| +\|+ \|\|++\|\|\| + | |
| Sbjct: | 119 | IVHRDLKPENILLDENGTVKIADFGLARKLESSSYEKLTTFVGTPEYMAPEVLEGRGYSS | 178 |
| NOV 3: | 309 | KVDLWSLGVLCCEFLVGKPPFEANTYQETYKRI---SRVEFTFPDFVTEGARDLISRLLK | 365 |
| | | \|\|\|+\|\|\|\|\|+ \| \| \|\| \|\| \| \|\| \|+ \| +\| +\|\|\| + \| | |
| Sbjct: | 179 | KVDVWSLGVILYELLTGKLPFPGIDPLEELFRIKERPRLRLPLPPNCSEELKDLIKKCLN | 238 |
| NOV 3: | 366 | HVPSQRPLMREVLEYPWI 383 | |
| | | \| +\|\| +\|+\| +\|\| | |
| Sbjct: | 239 | KDPEKRPTAKEILNHPWF 256 | |

TABLE 4G
Domain Analysis of NOV4

gnl|Smart|smart00219, TyrKc, Tyrosine kinase, catalytic domain;
Phosphotransferases. Tyrosine-specific kinase subfamily (SEQ ID NO:100)
CD-Length = 258 residues, 99.6% aligned
Score = 127 bits (318), Expect = 2e−30

| | | | |
|---|---|---|---|
| NOV 3: | 133 | LEIGRPPGKGKFGNVYLAREKQSKFILALRVLFKAQLEKAGVEHQ--LRREVEIQSHLQH | 190 |
| | | \| +\|+ \|+\| \|\| \|\| \| + + \| \| \| \| + \|\| + \| \| | |
| Sbjct: | 1 | LTLGKKLGEGAFGEVYKGTLKGKGGVE-VEVAVKTLKEDASEQQIEEFLREARLMRKLDH | 59 |
| NOV 3: | 191 | PNIIRLYGYFHDATRVYLILEYTPLETVNTELQKLSK--FDEQRTATYITELASALSYCH | 248 |
| | | \|\|\|++\| \| + + +++\|\| + \|+\| ++ ++\| + \| | |
| Sbjct: | 60 | PNIVKLLGVCTEEEPLMIVMEYMEGGDLLDYLRKNRPKELSLSDLLSFALQIARGMEYLE | 119 |
| NOV 3: | 249 | SKTVIHRDIKPENLLLGSAGELEIANFGWSEHAPSSRRTTLCGTLD----YLPPEMIEGR | 304 |
| | | \|\| +\|\|\|+ \| \|+\| ++\|\|+\|\| + + ++ \|\| ++ | |
| Sbjct: | 120 | SKNFVHRDLAARNCLVGENKTVKIADFGLARDLYDDDYYRKKKSPRLPIRWMAPESLKDG | 179 |
| NOV 3: | 305 | MHDEKVDLWSLGVLCCE-FLVGKPPFEANTYQETYKRISRVEF-TFPDFVTEGARDLISR | 362 |
| | | \| \|+\|\| \|\|\| \| \| +\|+ \|+ + +\| + + + \| + \|\|+ + | |
| Sbjct: | 180 | KFTSKSDVWSFGVLLWEIFTLGESPYPGMSNEEVLEYLKKGYRLPQPPNCPDEIYDLMLQ | 239 |
| NOV 3: | 363 | LLKHVPSQRPMLREVLEY 380 | |
| | | \| \|\| \|++\| | |
| Sbjct: | 240 | CWAEDPEDRPTFSELVER 257 | |

Amplification of chromosome 20q DNA has been reported in a variety of cancers. DNA amplification on 20q13 has also been correlated with poor prognosis among axillary node-negative breast tumor cases. Sen et al. (1997) cloned a partial cDNA encoding STK15 (also known as BTAK and aurora2) from this amplicon and found that it is amplified and overexpressed in 3 human breast cancer cell lines. STK15 encodes a centrosome-associated kinase. Zhou et al. (1998) found that STK15 is involved in the induction of centrosome duplication-distribution abnormalities and aneuploidy in mammalian cells. Centrosomes appear to maintain genomic stability through the establishment of bipolar spindles during cell division, ensuring equal segregation of replicated chromosomes to 2 daughter cells. Deregulated duplication and distribution of centrosomes are implicated in chromosome segregation abnormalities, leading to aneuploidy seen in many cancer cell types. Zhou et al. (1998) found amplification of STK15 in approximately 12% of primary breast tumors, as well as in breast, ovarian, colon, prostate, neuroblastoma, and cervical cancer cell lines. Additionally, high expression of STK15 mRNA was detected in tumor cell lines without evidence of gene amplification. Ectopic expression of STK15 in mouse NIH 3T3 cells led to the appearance of abnormal centrosome number (amplification) and transformation in vitro. Finally, overexpression of STK15 in near-diploid human breast epithelial cells revealed similar centrosome abnormality, as well as induction of aneuploidy. These findings suggested that STK15 is a critical kinase-encoding gene, whose overexpression leads to centrosome amplification, chromosomal instability, and transformation in mammalian cells. Zhou et al. (1998) found that the open reading frame of the full-length STK15 cDNA sequence encodes a 403-amino acid protein with a molecular mass of approximately 46 kD. STK6 (602687), also referred to as AIK, is highly homologous to STK15. The Drosophila ‘aurora’ and S. cerevisiae Ipl1 STKs are involved in mitotic events such as centrosome separation and chromosome segregation. Using a degenerate primer-based PCR method to screen for novel STKs, Shindo et al. (1998) isolated mouse and human cDNAs encoding STK15, which they termed ARK1 (aurora-related kinase-1). Cell cycle and Northern blot analyses showed that peak expression of STK15 occurs during the G2/M phase and then decreases. By interspecific backcross mapping, Shindo et al. (1998) mapped the mouse Stk15 gene to the distal region of chromosome 2 in a region showing homology of synteny with human 20q The disclosed NOV4 nucleic acid of the invention encoding a Aurora-related kinase 1-like protein includes the nucleic acid whose sequence is provided in Table 4A or a fragment thereof. The invention also includes a mutant or variant nucleic acid any of whose bases may be changed from the corresponding base shown in Table 4A while still encoding a protein that maintains its Aurora-related kinase 1-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 5 percent of the bases may be so changed.

The disclosed NOV4 protein of the invention includes the Aurora-related kinase 1-like protein whose sequence is provided in Table 4B. The invention also includes a mutant or variant protein any of whose residues may be changed from the corresponding residue shown in Table 4B while still encoding a protein that maintains its Aurora-related kinase 1-like activities and physiological functions, or a functional fragment thereof. In the mutant or variant protein, up to about 9 percent of the residues may be so changed.

The protein similarity information, expression pattern, and map location for the Aurora-related kinase 1-like protein and nucleic acid (NOV4) disclosed herein suggest that NOV4 may have important structural and/or physiological functions characteristic of the citron kinase-like family. Therefore, the NOV4 nucleic acids and proteins of the invention are useful in potential diagnostic and therapeutic applications. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo.

The NOV4 nucleic acids and proteins of the invention are useful in potential diagnostic and therapeutic applications implicated in various diseases and disorders described below. For example, the compositions of the present invention will have efficacy for treatment of patients suffering from breast, ovarian, colon, prostate, neuroblastoma, and cervical cancer, Cardiomyopathy, Atherosclerosis, Hypertension, Congenital heart defects, Aortic stenosis, Atrial septal defect (ASD), Atrioventricular (A-V) canal defect, Ductus arteriosus, Pulmonary stenosis, Subaortic stenosis, Ventricular septal defect (VSD), valve diseases, Tuberous sclerosis, Scleroderma, Obesity, Transplantation, Diabetes, Von Hippel-Lindau (VHL) syndrome, Pancreatitis, Alzheimer's disease, Stroke, hypercalceimia, Parkinson's disease, Huntington's disease, Cerebral palsy, Epilepsy, Lesch-Nyhan syndrome, Multiple sclerosis, Ataxia-telangiectasia, Leukodystrophies, Behavioral disorders, Addiction, Anxiety, Pain, and Neuroprotection and/or other pathologies. The NOV4 nucleic acid, or fragments thereof, may further be useful in diagnostic applications, wherein the presence or amount of the nucleic acid or the protein are to be assessed.

NOV4 nucleic acids and polypeptides are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. For example the disclosed NOV4 protein have multiple hydrophilic regions, each of which can be used as an immunogen. In one embodiment, contemplated NOV4 epitope is from about amino acids 1 to 10. In another embodiment, a NOV4 epitope is from about amino acids 15 to 160. In additional embodiments, NOV4 epitopes are from about amino acids 175 to 210, from about amino acids 220 to 240, from about amino acids 250 to 270, from about amino acids 280 to 320, from about amino acids 340 to 375, and from about amino acids 380 to 400. This novel protein also has value in development of powerful assay system for functional analysis of various human disorders, which will help in understanding of pathology of the disease and development of new drug targets for various disorders.

NOV5

A disclosed NOV5 nucleic acid of 1500 nucleotides (designated CuraGen Acc. No. SC44326718_A) encoding a novel 26S protease regulatory subunit 4-like protein is shown in Table 5A. An open reading frame was identified beginning with an ATG initiation codon at nucleotides 101-103 and ending with a TAG codon at nucleotides 1427-1429. A putative untranslated region downstream from the termination codon is underlined in Table 5A, and the start and stop codons are in bold letters.

TABLE 5A
NOV5 Nucleotide Sequence

(SEQ ID NO:15)

GTATCCCCAAGAGAAAATACGCATCAAAAATTAGGAACTTAGAAATGATAGTTGAGGTGGAGGAACTTCC
AGCAGTGGCAGCTCAAGTGGCCAAGACAAGATGGGTCAAAGTCAGGGTGATGGTCATGGTCCTAGACGTG
GCAAGAAGGATGAAAAGGACAAGAAAAATAAGTACGAACCTCTTGTACCAACTAGAGTGGCGGAAAAAGA
AGAAAAAACAAAGGGACAAGATGTTGCCAGTAAACTGCCACTGGTGACACTTCACACTCAGTGTCGGTTA
AAATTACTGAAGTTAGAGAGAATTAAAGACTACCTTCTCATGGTGGAAGAATTCATTAGAAATCAGGAAC
AAATAAAACTATTAGAAGAAAAGCAAGAGGAGGGAAGATCAAAAGTGGATGATCTGAGGGGGACCCCAAT
GTCAGTAGGAAACTTGGAAGAGATCATCGATGACAATCATGCCATTGTGTCTACATCTGTGGGCTCAGAA
CACTATGACAGCATTATTTCATTTGTAGAGAAGGATCTGCTGGAACCTGGCTGCTCGATTCTGCTCAGAC
ACAAGGTACATGCGGTGATAGGGGTGCTGATGGATGATACGGGTCCCCTGGTCACAATGATGAAGGTGGA
GAAGGCCCCCCAGGAGACCTATGTCAATACTGGGGGGTTGGACAACCAAATTCAGGAAATTAAGGAATCT
ATGGAGCTTCCTCTCCCCCATCCTGAATATTATGAAGAGATGGGTACAAAGCCTCCTAAAGGGGTCATTC
TCTGTGGTCCACCTGGCACAGGTAAAACCTTGTTAGCCAAAGCAGTAGCAAACCAAACCTCAGCCACTTT
CTTGAGAGTGGTTGGCTCTGAACTTATTCAGAAGTACCTAGGTGATGGGCCCAAACTCGTACGGCAAGTA
TTTCAAGTTGCTGAAGAACATGCACCATCCATCATGTTTACTGATGAAATTGAAGCCATTGGGACAAAAA
GATATGACTCCAATTCTGGTGGTGAGAGAGAAATTCAGCAAACAATGTTGGAATTGGAACTGTTGAACCA
ATTGGGTGGATTTGATTCTAGGGAAGATGTGAAAGTTATCATGGCCACAAAACAAGTAGAAACTTTGGAT
CCAGTACTTATCAGACCAGGCCGCATTGACAAGAAGATCGAGTTCCACCTGCCTGATGAAAAGACTAAGA
AGCACATCTTTCAGATTCACACAAGCAGGATGACACTGGCCAATGATGTAACCCTGGACGACTTGATCAT
GGCTAAAGATGACTTCTCTGGTGCTGACATCAAGGCAATCTGTACAGAAGCTGGTCTGATGGCCTTAAGA
GAACATAGAATGAAAGCAACAAATGAAGACTTCAAAAAATCTATAGAAAGTGTTCTTTATAAGAAACACG
AAGGCATCCCTGAGGGGCTTTATCTCTAGTGAACCACCGCTGCCATCAGGAAGATGGTTGGGAGATTTCC
CAACCCCTGAAAGGGATGAGGTTGGGGGAG

The nucleic acid sequence NOV5, located on chromosome 5 has 1347 of 1447 bases (93%) identical to a gb:GENBANK-ID:HUM26SPSIV|acc:L02426 mRNA from Homo sapiens (Human 26S protease (S4) regulatory subunit mRNA, complete cds (E=2.4e⁻²⁷⁷).

A NOV5 polypeptide (SEQ ID NO: 16) encoded by SEQ ID NO: 15 is 442 amino acid residues and is presented using the one letter code in Table 5B. Signal P, Psort and/or Hydropathy results predict that NOV5 has no signal peptide and is likely to be localized in the cytoplasm with a certainty of 0.4500. In other embodiments, NOV5 may also be localized to the microbody (peroxisome) with a certainty of 0.3000, the mitochondrial matrix space with a certainty of 0.1000, or the lysosome (lumen) with a certainty of 0.1000.

TABLE 5B
NOV5 protein sequence

(SEQ ID NO:16)

MGQSQGDGHGPRRGKKDEKDKKNKYEPLVPTRVAEKEEKTKGQDVASKLP
LVTLHTQCRLKLLKLERIKDYLLMVEEFIRNQEQIKLLEEKQEEGRSKVD
DLRGTPMSVGNLEEIIDDNHAIVSTSVGSEHYDSIISFVEKDLLEPGCSI
LLRHKVHAVIGVLMDDTGPLVTMMKVEKAPQETYVNTGGLDNQIQEIKES
MELPLPHPEYYEEMGTKPPKGVILCGPPGTGKTLLAKAVANQTSATFLRV
VGSELIQKYLGDGPKLVRQVFQVAEEHAPSIMFTDEIEAIGTKRYDSNSG
GEREIQQTMLELELLNQLGGFDSREDVKVIMATKQVETLDPVLIRPGRID
KKIEFHLPDEKTKKHIFQIHTSRMTLANDVTLDDLIMAKDDFSGADIKAI
CTEAGLMALREHRMKATNEDFKKSIESVLYKKHEGIPEGLYL

The full amino acid sequence of the protein of the invention was found to have 383 of 442 amino acid residues (86%) identical to, and 405 of 442 amino acid residues (91%) similar to, the 440 amino acid residue ptnr:SWISSPROT-ACC:P49014 protein from Mus musculus (Mouse), and Rattus norvegicus (Rat) (26S Protease Regulatory Subunit 4 (P26S4) (E=1.7e⁻²⁰⁰)

NOV5 also has homology to the amino acid sequences shown in the BLASTP data listed in Table 5C.

TABLE 5C
BLAST results for NOV5

| Gene Index/ | | Length | Identity | Positives | |
|---|---|---|---|---|---|
| Identifier | Protein/Organism | (aa) | (%) | (%) | Expect |
| gi\|4506207\|ref\|NP_002793.1\| | proteasome | 440 | 382/442 | 405/442 | 0.0 |
| (NM_002802) | (prosome, | | (86%) | (91%) | |
| | macropain) 26S | | | | |
| | subunit, ATPase, | | | | |
| | 1; Proteasome 26S | | | | |
| | subunit, ATPase, 1 | | | | |
| | [Homo sapiens] | | | | |
| gi\|6679501\|ref\|NP_032973.1\| | protease (prosome, | 440 | 383/442 | 405/442 | 0.0 |
| (NM_008947) | macropain) 26S | | (86%) | (90%) | |
| | subunit, ATPase 1 | | | | |
| | [Mus musculus] | | | | |
| gi\|345717\|pir\|\|A44468 | 26S proteasome | 440 | 381/442 | 404/442 | 0.0 |
| | regulatory chain 4 | | (86%) | (91%) | |
| | [validated] - | | | | |
| | human | | | | |
| gi\|16741033\|gb\|AAH16368.1\| | protease (prosome, | 440 | 382/442 | 404/442 | 0.0 |
| AAH16368 | macropain) 26S | | (86%) | (90%) | |
| (BC016368) | subunit, ATPase 1 | | | | |
| | [Homo sapiens] | | | | |
| gi\|2492516\|sp\|Q90732\| | 26S PROTEASE | 440 | 378/442 | 402/442 | 0.0 |
| PRS4_CHICK | REGULATORY SUBUNIT | | (85%) | (90%) | |
| | 4 (P26S4) | | | | |

The homology of these sequences is shown graphically in the ClustalW analysis shown in Table SD. V,23-24/2

Tables 5E-F list the domain description from DOMAIN analysis results against NOV5. This indicates that the NOV5 sequence has properties similar to those of other proteins known to contain this domain.

TABLE 5E
Domain Analysis of NOV5

gnl|pfam|pfam00004, AAA, ATPase family associated with various
cellular activities (AAA). AAA family proteins often perform
chaperone-like functions that assist in the assembly, operation, or
disassembly of protein complexes (SEQ ID NO:101)
CD-Length = 186 residues, 100.0% aligned
Score = 190 bits (483), Expect = 1e−49

| | | | |
|---|---|---|---|
| NOV 4: | 221 | GVILCGPPGTGKTLLAKAVANQTSATFLRVVGSELIQKYLGDGPKLVRQVFQVAEEHAPS | 280 |
| | | \|++\| \|\|\|\|\|\|\|\|\|\|\|\|\|\|\| + \|+ + \|\|\|\|+ \|\|+\|+ \|\|\|\| +\| +\| + \|\| | |
| Sbjct: | 1 | GILLYGPPGTGKTLLAKAVAKELGVPFIEISGSELLSKYVGESEKLVRALFSLARKSAPC | 60 |
| NOV 4: | 281 | IMFTDEIEAIGTKRYDSNSGGEREIQQTMLELELLNQLGGFDSREDVKVIMATKQVETLD | 340 |
| | | \|+\| \|\|\|+\|+ \|\| \| +\| + +\|\| ++ \|\|+ +\| \|\| \|\| + + \|\| | |
| Sbjct: | 61 | IIFIDEIDALAPKRGDVGTGDVSS----RVVNQLLTEMDGFEKLSNVIVIGATNRPDLLD | 116 |
| NOV 4: | 341 | PVLIRPGRIDKKIEFHLPDEKTKKHIFQIHTSRMTLANDVTLDDLIMAKDDFSGADIKAI | 400 |
| | | \| \|+\|\|\|\| \|++\|\| \|\|\|\|+ + \| +\|\| + \| \|\| \|\|++ \|\|\|\|\|+ \|+ | |
| Sbjct: | 117 | PALLRPGRFDRRIEVPLPDEEERLEILKIHLKKKPLEKDVDLDEIARRTPGFSGADLAAL | 176 |
| NOV 4: | 401 | CTEAGLMALR 410 | |
| | | \| \|\| \| \|+\| | |
| Sbjct: | 177 | CREAALRAIR 186 | |

TABLE 5F
Domain Analysis of NOV5

gnl|Smart|smart00382, AAA, ATPases associated with a variety of
cellular activities; AAA. This profile/alignment only detects a
fraction of this vast family. The poorly conserved N-terminal helix is
missing from the alignment. (SEQ ID NO:102)
CD-Length = 151 residues, 100.0% aligned
Score = 61.6 bits (148), Expect = 9e−11

| | | | |
|---|---|---|---|
| NOV 4: | 218 | PPKGVILCGPPGTGKTLLAKAVANQTSATFLRVV-------------------GSELIQK | 258 |
| | | \| + \|++ \|\|\|\|+\|\|\| \|\|+\|+\| + \|+ \| | |
| Sbjct: | 1 | PGEVVLIVGPPGSGKTTLARALARELGPDGGGVIYIDGEDLREEALLQLLRLLVLVGEDK | 60 |
| NOV 4: | 259 | YLGDGPKLVRQVFQVAEEHAPSIMFTDEIEAIGTKRYDSNSGGEREIQQTMLELELLNQL | 318 |
| | | \| \| + +\| +\| + \| ++ \|\|\| ++ + +\| \|\|\| \| | |
| Sbjct: | 61 | LSGSGGQRIRLALALARKLKPDVLILDEITSLLDAEQE---------ALLLLLEELLRLL | 111 |
| NOV 4: | 319 | GGFDSREDVKVIMATKQVETLDPVLIRPGRIDKKIEFHLPD 359 | |
| | | \|+\| \|\| \| \| \| \|+\| \| \|++\| | |
| Sbjct: | 112 | LLLLKEENVTVIATTNDETDLIPALLRR-RFDRRIVLLRIL 151 | |

Ubiquitinated proteins are degraded by a 26S ATP-dependent protease. The protease is composed of a 20S catalytic proteasome and 2 PA700 regulatory modules. The PA700 complex is composed of multiple subunits, including at least 6 related ATPases and approximately 15 non-ATPase polypeptides. Tanahashi et al. (1998) stated that each of the 6 ATPases, namely PSMC1, PSMC2 (154365), PSMC3 (186852), PSMC4 (602707), PSMC5 (601681), and PSMC6 (602708), contains an AAA (ATPases associated with diverse cellular activities) domain (see PSMC5). Dubiel et al. (1992) cloned cDNAs encoding subunit 4 (S4) of the 26S protease by screening a HeLa cell cDNA library with probes that were produced using the protein sequence. The 440-amino acid protein has a molecular mass of 51 kD by SDS-PAGE. By fluorescence in situ hybridization, Tanahashi et al. (1998) mapped the human PSMC1 gene to 19p13.3. Hoyle and Fisher (1996) found that the human and mouse PSMC1 proteins have 99% amino acid identity. They reported that the mouse Psmc1 gene contains at least 11 exons. By analysis of an interspecific backcross, Hoyle and Fisher (1996) mapped the mouse Psmc1 gene to chromosome 12. Nomenclature note: The PSMC1 gene product, which Dubiel et al. (1992) called subunit 4 (S4), is distinct from the PSMC4 (602707) gene product.

The disclosed NOV5 nucleic acid of the invention encoding a 26S protease regulatory subunit 4-like protein includes the nucleic acid whose sequence is provided in Table 5A or a fragment thereof. The invention also includes a mutant or variant nucleic acid any of whose bases may be changed from the corresponding base shown in Table 5A while still encoding a protein that maintains its 26S protease regulatory subunit 4-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 7 percent of the bases may be so changed.

The disclosed NOV5 protein of the invention includes the 26S protease regulatory subunit 4-like protein whose sequence is provided in Table 5B. The invention also includes a mutant or variant protein any of whose residues may be changed from the corresponding residue shown in Table 5B while still encoding a protein that maintains its 26S protease regulatory subunit 4-like activities and physiological functions, or a functional fragment thereof. In the mutant or variant protein, up to about 14 percent of the residues may be so changed.

The protein similarity information, expression pattern, and map location for the 26S protease regulatory subunit 4-like protein and nucleic acid (NOV5) disclosed herein suggest that this NOV5 protein may have important structural and/or physiological functions characteristic of the 26S protease regulatory subunit 4 family. Therefore, the NOV5 nucleic acids and proteins of the invention are useful in potential diagnostic and therapeutic applications. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo.

The NOV5 nucleic acids and proteins of the invention are useful in potential diagnostic and therapeutic applications implicated in various diseases and disorders described below. For example, the compositions of the present invention will have efficacy for treatment of patients suffering from cataract and Aphakia, Alzheimer's disease, neurodegenerative disorders, inflammation and modulation of the immune response, viral pathogenesis, aging-related disorders, neurologic disorders, cancer and/or other pathologies. The NOV5 nucleic acids, or fragments thereof, may further be useful in diagnostic applications, wherein the presence or amount of the nucleic acid or the protein are to be assessed.

NOV5 nucleic acids and polypeptides are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. For example, the disclosed NOV5 protein has multiple hydrophilic regions, each of which can be used as an immunogen. In one embodiment, a contemplated NOV5 epitope is from about amino acids 5 to 50. In another embodiment, a NOV5 epitope is from about amino acids 75 to 125. In additional embodiments, NOV5 epitopes are from about amino acids 175 to 225, from about amino acids 280 to 320, from about amino acids 330 to 380, and from about amino acids 390 to 440. These novel proteins can be used in assay systems for functional analysis of various human disorders, which will help in understanding of pathology of the disease and development of new drug targets for various disorders.

NOV6

A disclosed NOV6 nucleic acid of 1020 nucleotides (also referred to as GMAC073364_A_da1) encoding a novel MITSUGUMIN29-like protein is shown in Table 6A. An open reading frame was identified beginning with an ATG initiation codon at nucleotides 2-4 and ending with a TAA codon at nucleotides 818-820. Putative untranslated regions upstream from the initiation codon and downstream from the termination codon are underlined in Table 6A, and the start and stop codons are in bold letters.

TABLE 6A
NOV6 Nucleotide Sequence

| | |
|---|---|
| CATGTCCTCGACCGAGAGCGCCGGCCGCACGGCGGACAAGTCGCCGCGCCAGCAGGTGGACC | (SEQ ID NO:17) |
| GCCTACTCGTGGGGCTGCGCTGGCGGCGGCTGGAGGAGCCGCTGGGCTTCATCAAAGTTCTC | |
| CAGTGGCTCTTTGCTATTTTCGCCTTCGGGTCCTGTGGCTCCTACAGCGGGGAGACAGGAGC | |
| AATGGTTCGCTGCAACAACGAAGCCAAGGACGTGAGCTCCATCATCGTTGCATTTGGCTATC | |
| CCTTCAGGTTGCACCGGATCCAATATGAGATGCCCCTCTGCGATGAAGAGTCCAGCTCCAAG | |
| ACCATGCACCTCATGGGGGACTTCTCTGCACCCGCCGAGTTCTTCGTGACCCTTGGCATCTT | |
| TTCCTTCTTCTATACCATGGCTGCCCTAGTTATCTACCTGCGCTTCCACAACCTCTACACAG | |
| AGAAGAAACGCTTCCCGCTGGTGGACTTCTGTGTGACTGTCTCCTTCACCTTCTTCTGGCTG | |
| GTAGCTGCAGCTGCCTGGGGCAAGGGCCTGACCGATGTCAAGGGGGCCACACGACCATCCAG | |
| CTTGACAGCAGCCATGTCAGTGTGCCATGGAGAGGAAGCAGTGTGCAGTGCCGGGGCCACGC | |
| CCTCTATGGGCCTGGCCAACATCTCCGTGCTCTTTGGCTTTATCAACTTCTTCCTGTGGGCC | |
| GGGAACTGTTGGTTTGTGTTCAAGGAGACCCCGTGGCATGGACAGGGCCAGGGCCAGGACCA | |
| GGACCAGGACCAGGACCAGGGCCAGGGTCCCAGCCAGGAGAGTGCAGCTGAGCAGGGAGCAG | |
| TGGAGAAGCAGTAAGCAGCCCCCCACCT | |

The NOV6 nucleic acid was identified on chromosome 3 and has 727 of 805 bases (90%) identical to a gb:GENBANK-ID:AB004816|acc:AB004816.1 mRNA from Oryctolagus cuniculus (Oryctolagus cuniculus mRNA for mitsugumin29, complete cds (E=2.5e⁻¹⁴²).

A disclosed NOV6 polypeptide (SEQ ID NO: 18) encoded by SEQ ID NO: 17 is 272 amino acid residues and is presented using the one-letter code in Table 6B. Signal P, Psort and/or Hydropathy results predict that NOV6 has a signal peptide and is likely to be localized on the plasma membrane with a certainty of 0.6000. In other embodiments, NOV6 may also be localized to the Golgi body with acertainty of 0.4000, the endoplasmic reticulum (membrane) with a certainty of 0.3000, or the nucleus with a certainty of 0.1000. The most likely celavage site for NOV6 is between positions 57 and 58, SYS-GE.

TABLE 6B
Encoded NOV6 protein sequence

(SEQ ID NO:18)

MSSTESAGRTADKSPRQQVDRLLVGLRWRRLEEPLGFIKVLQWLFAIFAF
GSCGSYSGETGAMVRCNNEAKDVSSIIVAFGYPFRLHRIQYEMPLCDEES
SSKTMHLMGDFSAPAEFFVTLGIFSFFYTMAALVIYLRFHNLYTENKRFP
LVDFCVTVSFTFFWLVAAAAWGKGLTDVKGATRPSSLTAAMSVCHGEEAV
CSAGATPSMGLANISVLFGFINFFLWAGNCWFVFKETPWHGQGQGQDQDQ
DQDQGQGPSQESAAEQGAVEKQ

The disclosed NOV6 amino acid sequence has 727 of 805 amino acid residues (90%) identical to, and 727 of 805 amino acid residues (90%) similar to, the 3489 amino acid residue gb:GENBANK-ID:AB004816|acc:AB004816.1 protein from Oryctolagus cuniculus (Oryctolagus cuniculus mRNA for mitsugumin29, complete cds) (E=2.5e⁻¹⁴²).

Based on the semi quantitative PCR, NOV6 is specially expressed in: Skeletal muscle, Heart, Kidney, Adrenal gland and one of the Lung cancer cell lines (Lung cancer NCI-H522) at a measurably higher level than the following tissues: Endothelial cells, Pancreas, Thyroid, Salivary gland, Pituitary gland, Brain (fetal), Brain (whole), Brain (amygdala), Brain (cerebellum), Brain (hippocampus), Brain (thalamus), Cerebral Cortex, Spinal cord, Bone marrow, Thymus, Spleen, Lymph node, Colorectal, Stomach, Small intestine, Bladder, Trachea, Kidney (fetal), Liver, Liver (fetal), Lung, Lung (fetal), Mammary gland, Ovary, Uterus, Placenta, Prostate, Testis, Melanoma, Adipose and cancer cell lines including Breast cancer, CNS cancer, Colon cancer, Gastric cancer, Lung cancer (except Lung cancer NCI-H522 ), Ovarian cancer, Pancreatic cancer, and Renal cancer.

In addition, NOV6 is predicted to be expressed in skeletal muscle because of the expression pattern of (GENBANK-ID: gb:GENBANK-ID:AB004816|acc:AB004816.1) a closely related mitsugumin29 homolog in Oryctolagus cuniculus.

NOV6 also has homology to the amino acid sequences shown in the BLASTP data listed in Table 6C.

TABLE 6C
BLAST results for NOV6

| Gene Index/ | | Length | Identity | Positives | |
|---|---|---|---|---|---|
| Identifier | Protein/Organism | (aa) | (%) | (%) | Expect |
| gi\|3077703\|dbj\|BAA25784.1\| | mitsugumin29 | 264 | 252/272 | 256/272 | e−136 |
| (AB004816) | [Oryctolagus | | (92%) | (93%) | |
| | cuniculus] | | | | |
| gi\|6678874\|ref\|NP_032622.1\| | mitsugumin 29 | 264 | 246/272 | 258/272 | e−133 |
| (NM_008596) | [Mus musculus] | | (90%) | (94%) | |
| gi\|12836843\|dbj\|BAB23831.1\| | putative [Mus | 285 | 118/251 | 158/251 | 7e−59 |
| (AK005132) | musculus] | | (47%) | (62%) | |
| gi\|1351168\|sp\|P20488\| | SYNAPTOPHYSIN | 307 | 109/221 | 145/221 | 3e−58 |
| SYPH_BOVIN | (MAJOR SYNAPTIC | | (49%) | (65%) | |
| | VESICLE PROTEIN | | | | |
| | P38) | | | | |
| gi\|2134413\|pir\|\|I50720 | synaptophysin IIa - | 268 | 109/217 | 142/217 | 4e−57 |
| | chicken | | (50%) | (65%) | |

The homology of these sequences is shown graphically in the ClustalW analysis shown in Table 6D.

[image]

[image]

Table 6E lists the domain description from DOMAIN analysis results against NOV6. This indicates that the NOV6 sequence has properties similar to those of other proteins known to contain this domain.

TABLE 6E
Domain Analysis of NOV6

gnl|Pfam|pfam01284, Synaptophysin, Synaptophysin/synaptoporin.
(SEQ ID NO:103)
CD-Length = 298 residues, 70.8% aligned
Score = 244 bits (622), Expect = 6e−66

| | | | |
|---|---|---|---|
| NOV 5: | 29 | RRLEEPLGFIKVLQWLFAIFAFGSCGSYSGETGAMVRCNNEAKDVSSIIVAFGYPFRLHR | 88 |
| | | + \|\|\|\|+\|\|\|\|\|+\|\|\|\|\|\| +\|\| \|\|\|\| \| \| \|+ + +\| +\|\| \|\|\|\|\|\| | |
| Sbjct: | 3 | MVIFAPLGFVKVLQWVFAIFAFATCGGYSGELQLSVDCANKTESDLNIDIAFAYPFRLHE | 62 |
| NOV 5: | 89 | IQYEMPLCDEESSSKTMHLMGDFSAPAEFFVTLGIFSFFYTMAALVIYLRFHNLYTENKR | 148 |
| | | + +\| \| \| \| \| + \|+\|\| \|+ \|\|\|\|\|\|+ +\|+\| \|++\|\|\| \|+ \| \| \| \|\| + | |
| Sbjct: | 63 | VTFEAPTC-EGDEKKNIALVGDSSSSAEFFVTVAVFAFLYSLAALATYIFFQNKYRENNK | 121 |
| NOV 5: | 149 | FPLVDFCVTVSFTFFWLVAAAAWGKGLTDVKGATRPSSLTAAMSVCHGEEAVCSAGATPS | 208 |
| | | \|\|+\|\| \| \| \| \|\|\| ++\|\| \|\|\|+\|\|\| \|\| \| + \| \|\| \| \| | |
| Sbjct: | 122 | GPLIDFIATAVFAFLWLVGSSAWAKGLSDVKMATDPEEIIKGMHACHQPGNKCKELHDPV | 181 |
| NOV 5: | 209 | MGLANISVLFGFINFFLWAGNCWFVFKETPWH 240 | |
| | | \| \| \|\|+\|\|\|+\|\| \|\|\|\|\| \|\|\|\|\|\|\| \| | |
| Sbjct: | 182 | MSGLNTSVVFGFLNFILWAGNIWFVFKETGWA 213 | |

In skeletal muscle, excitation-contraction (E-C) coupling requires the conversion of the depolarization signal of the invaginated surface membrane, namely the transverse (T-) tubule, to Ca2+ release from the sarcoplasmic reticulum (SR) (Takeshima H et al., Biochem J April 1998 1;331 (Pt 1):317-22/PMID: 9512495, UI: 98180964). Signal transduction occurs at the junctional complex between the T-tubule and SR, designated as the triad junction, which contains two components essential for E-C coupling, namely the dihydropyridine receptor as the T-tubular voltage sensor and the ryanodine receptor as the SR Ca2+-release channel. However, functional expression of the two receptors seemed to constitute neither the signal-transduction system nor the junction between the surface and intracellular membranes in cultured cells, suggesting that some as-yet-unidentified molecules participate in both the machinery. In addition, the molecular basis of the formation of the triad junction is totally unknown. It is therefore important to examine the components localized to the triad junction. Takeshima et al. report the identification using monoclonal antibody and primary structure by cDNA cloning of mitsugumin29, a novel transmembrane protein from the triad junction in skeletal muscle. This protein is homologous in amino acid sequence and shares characteristic structural features with the members of the synaptophysin family. The subcellular distribution and protein structure suggest that mitsugumin29 is involved in communication between the T-tubular and junctional SR membranes.

Physiological roles of the members of the synaptophysin family, carrying four transmembrane segments and being basically distributed on intracellular membranes including synaptic vesicles, have not been established yet (Nishi M et al., J Cell Biol December 1998 27;147(7):1473-80/PMID: 10613905, UI: 20082885). Recently, mitsugumin29 (MG29) was identified as a novel member of the synaptophysin family from skeletal muscle. MG29 is expressed in the junctional membrane complex between the cell surface transverse (T) tubule and the sarcoplasmic reticulum (SR), called the triad junction, where the depolarization signal is converted to Ca(2+) release from the SR. In this study, Nishi et al. examined biological functions of MG29 by generating knockout mice. The MG29-deficient mice exhibited normal health and reproduction but were slightly reduced in body weight. Ultrastructural abnormalities of the membranes around the triad junction were detected in skeletal muscle from the mutant mice, i.e., swollen T tubules, irregular SR structures, and partial misformation of triad junctions. In the mutant muscle, apparently normal tetanus tension was observed, whereas twitch tension was significantly reduced. Moreover, the mutant muscle showed faster decrease of twitch tension under Ca(2+)-free conditions. The morphological and functional abnormalities of the mutant muscle seem to be related to each other and indicate that MG29 is essential for both refinement of the membrane structures and effective excitation-contraction coupling in the skeletal muscle triad junction. These results further imply a role of MG29 as a synaptophysin family member in the accurate formation of junctional complexes between the cell surface and intracellular membranes.

The temporal appearance and subcellular distribution of mitsugumin29 (MG29), a 29-kDa transmembrane protein isolated from the triad junction in skeletal muscle, were examined by immunohistochemistry during the development of rabbit skeletal muscle (Komazaki S et al., Dev Dyn June 1999 ;215(2):87-95/PMID: 10373013, UI: 99300228). MG29 appeared in the sarcoplasmic reticulum (SR) in muscle cells at fetal day 15 before the onset of transverse tubule (T tubule) formation. In muscle cells at fetal day 27, in which T tubule and triad formation is ongoing, both SR and triad were labeled for MG29. In muscle cells at newborn 1 day, the labeling of the SR had become weak and the triads were well developed and clearly labeled for MG29. Specific and clear labeling for MG29 was restricted to the triads in adult skeletal muscle cells. When MG29 was expressed in amphibian embryonic cells by injection of the cRNA, a large quantity of tubular smooth-surfaced endoplasmic reticulum (sER) was formed in the cytoplasm. The tubular sER was 20-40 nm in diameter and appeared straight or reticular in shape. The tubular sER was formed by the fusion of coated vesicles [budded off from the rough-surfaced endoplasmic reticulum (rER)] and vacuoles of rER origin. The present results suggest that MG29 may play important roles both in the formation of the SR and the construction of the triads during the early development of skeletal muscle cells.

Recently mitsugumin29 unique to the triad junction in skeletal muscle was identified as a novel member of the synaptophysin family; the members of this family have four transmembrane segments and are distributed on intracellular vesicles. In this study, Shimuta et al. FEBS Lett July 1998 17;431(2):263-7/PMID: 9708916, UI: 98372647, isolated and analyzed mouse mitsugumin29 cDNA and genomic DNA containing the gene. The mitsugumin29 gene mapped to the mouse chromosome 3 F3-H2 is closely related to the synaptophysin gene in exon-intron organization, which indicates their intimate relationship in molecular evolution. RNA blot hybridization and immunoblot analysis revealed that mitsugumin29 is expressed abundantly in skeletal muscle and at lower levels in the kidney. Immunofluorescence microscopy demonstrated that mitsugumin29 exists specifically in cytoplasmic regions of the proximal and distal tubule cells in the kidney. The results obtained may suggest that mitsugumin29 is involved in the formation of specialized endoplasmic reticulum systems in skeletal muscle and renal tubule cells.

The disclosed NOV6 nucleic acid of the invention encoding a MITSUGUMIN29-like protein includes the nucleic acid whose sequence is provided in Table 6A or a fragment thereof. The invention also includes a mutant or variant nucleic acid any of whose bases may be changed from the corresponding base shown in Table 6A while still encoding a protein that maintains its MITSUGUMIN29-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 10 percent of the bases may be so changed.

The disclosed NOV6 protein of the invention includes the MITSUGUMIN29-like protein whose sequence is provided in Table 6B. The invention also includes a mutant or variant protein any of whose residues may be changed from the corresponding residue shown in Table 6B while still encoding a protein that maintains its MITSUGUMIN29-like activities and physiological functions, or a functional fragment thereof. In the mutant or variant protein, up to about 10 percent of the residues may be so changed.

The NOV6 nucleic acids and proteins of the invention are useful in potential therapeutic applications implicated in eye/lens disorders including but not limited to muscular dystrophy, Lesch-Nyhan syndrome, myasthenia gravis, diabetes, autoimmune disease, renal artery stenosis, interstitial nephritis, glomerulonephritis, polycystic kidney disease, systemic lupus erythematosus, renal tubular acidosis, IgA nephropathy, hypercalceimia, cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, obesity, transplantation, adrenoleukodystrophy, congenital adrenal hyperplasia, and other diseases, disorders and conditions of the like. Also since the invention is highly expressed in one of the lung cancer cell lines (Lung cancer NCI-H522 ), it may be useful in diagnosis and treatment of this cancer. The NOV6 nucleic acid, or fragments thereof, may further be useful in diagnostic applications, wherein the presence or amount of the nucleic acid or the protein are to be assessed.

NOV6 nucleic acids and polypeptides are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. For example the disclosed NOV6 protein have multiple hydrophilic regions, each of which can be used as an immunogen. In one embodiment, contemplated NOV6 epitope is from about amino acids 10 to 40. In other embodiments, NOV6 epitope is from about amino acids 60 to 70, from about amino acids 90 to 130, from about amino acids 145 to 155, from about amino acid 170 to 180, and from about amino acids 220 to 270. This novel protein also has value in development of powerful assay system for functional analysis of various human disorders, which will help in understanding of pathology of the disease and development of new drug targets for various disorders.

NOV7

A disclosed NOV7 nucleic acid of 1020 nucleotides (also referred to as 106973211_EXT) encoding a novel Wnt-15-like protein is shown in Table 7A. An open reading frame was identified beginning with an CTG initiation codon at nucleotides 2-4 and ending with a TAG codon at nucleotides 995-997. A putative untranslated region upstream from the initiation codon and downstream from the termination codon is underlined in Table 7A, and the start and stop codons are in bold letters. Since the starting codon is not a traditional initiation codon, NOV7 could represent a partial reading frame, and could further extend in the 5′ direction.

TABLE 7A
NOV7 Nucleotide Sequence

(SEQ ID NO:19)

CCTGACCGGGCGGGAAGTCCTGACGCCCTTCCCAGGATTGGGCACTGCGGCAGCCCCGGCACAGGGCGGG
GCCCACCTGAAGCAGTGTGACCTGCTGAAGCTGTCCCGGCGGCAGAAGCAGCTCTGCCGGAGGGAGCCCG
GCCTGGCTGAGACCCTGAGGGATGCTGCGCACCTCGGCCTGCTTGAGTGCCAGTTTCAGTTCCGGCATGA
GCGCTGGAACTGTAGCCTGGAGGGCAGGATGGGCCTGCTCAAGAGAGGCTTCAAAGAGACAGCTTTCCTG
TACGCGGTGTCCTCTGCCGCCCTCACCCACACCCTGGCCCGGGCCTGCAGCGCTGGGCGCATGGAGCGCT
GCACCTGTGATGACTCTCCGGGGCTGGAGAGCCGGCAGGCCTGGCAGTGGGGCGTGTGCGGTGACAACCT
CAAGTACAGCACCAAGTTTCTGAGCAACTTCCTGGGGTCCAAGAGAGGAAACAAGGACCTGCGGGCACGG
GCAGAGGCCCACAATACCCACGTGGGCATCAAGGCTGTGAAGAGTGGCCTCAGGACCACGTGTAAGTGCC
ATGGCGTATCAGGCTCCTGTGCCGTGCGCACCTGCTGGAAGCAGCTCTCCCCGTTCCGTGAGACGGGCCA
GGTGCTGAAACTGCGCTATGACTCGGCTGTCAAGGTGTCCAGTGCCACCAATGAGGCCTTGGGCCGCCTA
GAGCTGTGGGCCCCTGCCAGGCAGGGCAGCCTCACCAAAGGCCTGGCCCCAAGGTCTGGGGACCTGGTGT
ACATGGAGGACTCACCCAGCTTCTGCCGGCCCAGCAAGTACTCACCTGGCACAGCAGGTAGGGTGTGCTC
CCGGGAGGCCAGCTGCAGCAGCCTGTGCTGCGGGCGGGGCTATGACACCCAGAGCCGCCTGGTGGCCTTC
TCCTGCCACTGCCAGGTGCAGTGGTGCTGCTACGTGGAGTGCCAGCAATGTGTGCAGGAGGAGCTTGTGT
ACACCTGCAAGCACTAGGCCTACTGCCCAGCAAGCCAGTC

The disclosed NOV7 nucleic acid sequence, located on chromosome 17, has 688 of 1009 bases (68%) identical to a gb:GENBANK-ID:AF031168|acc:AF031168.1 mRNA from Gallus gallus (Gallus gallus Wnt-14 protein (Wnt-14) mRNA, complete cds) (E=3.0e⁻⁷⁶).

A disclosed NOV7 polypeptide (SEQ ID NO: 20) encoded by SEQ ID NO: 19 is 331 amino acid residues and is presented using the one-letter amino acid code in Table 7B. Signal P, Psort and/or Hydropathy results predict that NOV7 contains no signal peptide and is likely to be localized in the cytoplasm with a certainty of 0.4500. In other embodiments, NOV7 is also likely to be localized to the microbody (peroxisome) with a certainty of 0.3000, the mtochondrial matrix space with a certainty of 0.1000, or to the lysosome (lumen) with a certainty of 0.1000.

TABLE 7B
Encoded NOV7 protein sequence.

(SEQ ID NO:20)

LTGREVLTPFPGLGTAAAPAQGGAHLKQCDLLKLSRRQKQLCRREPGLAE
TLRDAAHLGLLECQFQFRHERWNCSLEGRMGLLKRGFKETAFLYAVSSAA
LTHTLARACSAGRMERCTCDDSPGLESRQAWQWGVCGDNLKYSTKFLSNF
LGSKRGNKDLRARADAHNTHVGIKAVKSGLRTTCKCHGVSGSCAVRTCWK
QLSPFRETGQVLKLRYDSAVKVSSATNEALGRLELWAPARQGSLTKGLAP
RSGDLVYMEDSPSFCRPSKYSPGTAGRVCSREASCSSLCCGRGYDTQSRL
VAFSCHCQVQWCCYVECQQCVQEELVYTCKH

The disclosed NOV7 amino acid sequence has 205 of 330 amino acid residues (62%) identical to, and 252 of 330 amino acid residues (76%) similar to, the 354 amino acid residue ptnr:SWISSPROT-ACC:042280 protein from Gallus gallus (Chicken) (WNT-14 Protein Precursor) (E=1.3e⁻¹¹⁴).

The tissue expression of NOV7 is predicted to be expressed in brain because of the expression pattern of (GENBANK-ID: gb:GENBANK-ID:AF031168|acc:AF031168.1) a closely related Gallus gallus Wnt-14 protein (Wnt-14) mRNA, complete cds homolog.

NOV7 also has homology to the amino acid sequences shown in the BLASTP data listed in Table 7C.

TABLE 7C
BLAST results for NOV7

| Gene Index/ | | Length | Identity | Positives | |
|---|---|---|---|---|---|
| Identifier | Protein/Organism | (aa) | (%) | (%) | Expect |
| gi\|16303264\|dbj\|BAB70499.1\| | WNT14B [Homo | 357 | 330/331 | 330/331 | e−175 |
| (AB063483) | sapiens] | | (99%) | (99%) | |
| gi\|3915306\|sp\|O42280\| | WNT-14 PROTEIN | 354 | 204/332 | 253/332 | e−109 |
| WN14_CHICK | PRECURSOR | | (61%) | (75%) | |
| gi\|15082261\|ref\|NP_{—} | wingless-type | 365 | 209/335 | 255/335 | e−108 |
| 003386.1\| | MMTV integration | | (62%) | (75%) | |
| (NM_003395) | site family, | | | | |
| | member 14 [Homo | | | | |
| | sapiens] | | | | |
| gi\|139748\|sp\|P10108\| | WNT-1 PROTEIN | 371 | 120/313 | 175/313 | 5e−58 |
| WNT1_XENLA | PRECURSOR (XWNT- | | (38%) | (55%) | |
| | 1) (XINT-1) | | | | |
| gi\|3024851\|sp\|O14905\| | WNT-15 PROTEIN | 120 | 120/120 | 120/120 | 2e−56 |
| WN15_HUMAN | | | (100%) | (100%) | |

The homology of these sequences is shown graphically in the ClustalW analysis shown in Table 7D.

[image]

[image]

Tables 7E and 7F list the domain descriptions from DOMAIN analysis results against NOV7. This indicates that the NOV7 sequence has properties similar to those of other proteins known to contain this domain.

TABLE 7E
Domain Analysis of NOV7

gnl|Pfam|pfam00110, writ, writ family. (SEQ ID NO:l04)
GD-Length = 313 residues, 97.8% aligned
Score = 268 bits (684), Expect = 5e−73

| | | | |
|---|---|---|---|
| NOV 6: | 34 | LSRRQKQLCRREPGLAETLRDAAHLGLLECQFQFRHERWNCSLEGRMGL-----LKRGFK | 88 |
| | | \|\| \|\|+\|\|\|\|\| \| + ++ + \| \| + \|\|\| \|\|\| \|\|\|\|\| \|+ + \|\|+\| + | |
| Sbjct: | 8 | LSPRQRQLCRRNPDVMASVSEGAQLAIQECQHQFRGRRWNCSTLDRLRVVFGKVLKKGTR | 67 |
| NOV 6: | 89 | ETAFLYAVSSAALTHTLARACSAGRMERCTCDDSPG-LESRQAWQWGVCGDNLKYSTKFL | 147 |
| | | \|\|\|\|+\|\|+\|\|\| + \| + \|\|\|\| \| +\| \| \|\| \| + +\|\|\|\| \| \|\|+++ +\| | |
| Sbjct: | 68 | ETAFVYAISSAGVAHAVTRACSEGELESCGCDYKKGPGGPQGSWQWGGCSDNVEFGIRFS | 127 |
| NOV 6: | 148 | SNFLGSKRGNKDLRARADAHNTHVGIKAVKSGLRTTCKCHGVSGSCAVRTCWKQLSPFRE | 207 |
| | | \|+ ++ +\| \|+ + \|\| \| \|\|\|\|\| +\| \|\|\|\|\|\|\|\|\|\|+++\|\|\| \| \|\| | |
| Sbjct: | 128 | REFVDARERERDARSLNNLHNNEAGRKAVKSHMRRECKCHGVSGSCSMKTCWLSLPDFRA | 187 |
| NOV 6: | 208 | TGQVLKLRYDSAVKVSSATNEALGRLELWAPARQGSLTKGLAPRSGDLVYMEDSPSFCR- | 266 |
| | | \| \|\| +\|\| \|++\| \| + \|\| + \| \|\|\|\|+\|\|\|\| +\| | |
| Sbjct: | 188 | VGDALKDKYDGAIRV---EPNKRGMGQGSAPRLVAKNPRFKPPTRSDLVYLEDSPDYCER | 244 |
| NOV 6: | 267 | -PSKYSPGTAGRVCSREA----SCSSLCCGRGYDTQSRLVAFSCHCQVQWCCYVECQQCV | 321 |
| | | \| \| \|\| \|\|\|\|++ + \| \|\|\|\|\|\|\|+\|\| \|+\|+ \|\|\|\|\|+\|++\| | |
| Sbjct: | 245 | DRSTGSLGTQGRVCNKTSKGLDGCELLCCGRGYNTQQVERTEKCNCKFHWCCYVKCEECQ | 304 |
| NOV 6: | 322 | QEELVYTCK 330 | |
| | | + \|+\|\|\| | |
| Sbjct: | 305 | EVVEVHTCK 313 | |

TABLE 7F
Domain Analysis of NOV7

gnl|Smart|smart00097, WNT1, found in Wnt-1 (SEQ ID NO:105)
CD-Length = 304 residues, 98.7% aligned
Score = 248 bits (632), Expect = Se−67

| | | | |
|---|---|---|---|
| NOV 6: | 34 | LSRRQKQLCRREPGLAETLRDAAHLGLLECQFQFRHERWNCSLEGRMGL----LKRGFKE | 89 |
| | | \|\|\|\|\|+\|\|\|\| \| + ++ + \| \|+ \|\|\| \|\|\| \|\|\|\|\| \| + \|++\| +\| | |
| Sbjct: | 5 | LSRRQRQLCRANPDVMASVAEGAQEGIEECQHQFRFRRWNCSTAGLASIFGKVLRQGTRE | 64 |
| NOV 6: | 90 | TAFLYAVSSAALTHTLARACSAGRMERCTCDDSPGLESRQAWQWGVCGDNLKYSTKFLSN | 149 |
| | | \|\|\|+\|\|+\|\|\| + \| + \|\|\|\| \| ++ \| \|\| \| + \|+\|\| \| \|\|+ + \| | |
| Sbjct: | 65 | TAFVYAISSAGVAHAVTRACSQGELDSCGCDYSKRGSGGRGWEWGGCSDNIDFGIGFSFR | 124 |
| NOV 6: | 150 | FLGSK-RGNKDLRARADAHNTHVGIKAVKSGLRTTCKCHGVSGSCAVRTCWKQLSPFRET | 208 |
| | | \|+ ++ \| \| \|\| + \|\| \| \|\|\| ++ \|\|\|\|\|\|\|\|\|\|+\|+\|\|\| \|\| \|\|\| | |
| Sbjct: | 125 | FVDARERRGSDARALMNLHNNEAGRLAVKKTMKRECKCHGVSGSCSVKTCWLQLPEFREI | 184 |
| NOV 6: | 209 | GQVLKLRYDSAVKVSSATNEALGRLELWAPARQGSLTKGLAPRSGDLVYMEDSPSFC--R | 266 |
| | | \| \|\| +\|\| \| +\| \| + \| + \|\|\|\|+\| \|\| \|\| | |
| Sbjct: | 185 | GDYLKEKYDGASEV-VLDKRGTRGLVPANRDFK-------PPTNTDLVYLESSPDFCEKN | 236 |
| NOV 6: | 267 | PSKYSPGTAGRVCSREA----SCSSLCCGRGYDTQSRLVAFSCHQWCCYVECQQCVQ | 322 |
| | | \| \| \|\| \|\|\|\|++ + \| \|\|\|\|\|\|\|+\|+ \| \|+\|+ \|\|\|\|\|+\|+\|\| + | |
| Sbjct: | 237 | PKTGSLGTQGRVCNKTSKGLDGCDLLCCGRGYNTEHVEVVERCNCKFHWCCYVKCKQCRE | 296 |
| NOV 6: | 323 | EELVYTCK 330 | |
| | | +\|\|\| | |
| Sbjct: | 297 | RVEKHTCK 304 | |

Wnt proteins constitute a large family of molecules involved in cell proliferation, cell differentiation and embryonic patterning. They are known to interact with the Frizzled family of receptors to activate two main intracellular signaling pathways regulating intracellular calcium levels and gene transcription. Early studies on Wnts implicated them in cell proliferation and tumorigenesis, which have been borne out by recent work using transgenic and null mutant mice. Wnts are involved in processes involved in mammary gland development and cancer. Recent studies have demonstrated that these molecules are critical to organogenesis of several systems, such as the kidney and brain. Wnts regulate the early development, i.e. neural induction, and their role persists in later stages of development as well as in the mature organ. An example of this is seen in the brain, where the loss of certain Wnts leads to the absence of critical regions of the brain, e.g. the hippocampus, involved in learning and memory, or the cerebellum, involved in motor function. Wnts have also been implicated in the genesis of degenerative diseases such as Alzheimer's disease. The protein encoded by the novel gene described herein may therefore play a role in cellular proliferation, differentiation, dysregulation, organogenesis and disease processes such as cancer, developmental defects etc.

A partial sequence corresponding to this novel protein, with homology to the chicken Wnt-14, has been deposited in GenBank with the nomenclature Wnt-15.

Alzheimer's disease (AD) is a neurodegenerative disease with progressive dementia accompanied by three main structural changes in the brain: diffuse loss of neurons; intracellular protein deposits termed neurofibrillary tangles (NFT) and extracellular protein deposits termed amyloid or senile plaques, surrounded by dystrophic neurites. Two major hypotheses have been proposed in order to explain the molecular hallmarks of the disease: The ‘amyloid cascade’ hypothesis and the ‘neuronal cytoskeletal degeneration’ hypothesis. While the former is supported by genetic studies of the early-onset familial forms of AD (FAD), the latter revolves around the observation in vivo that cytoskeletal changes—including the abnormal phosphorylation state of the microtubule associated protein tau—may precede the deposition of senile plaques. Recent studies have suggested that the trafficking process of membrane associated proteins is modulated by the FAD-linked presenilin (PS) proteins, and that amyloid beta-peptide deposition may be initiated intracellularly, through the secretory pathway. Current hypotheses concerning presenilin function are based upon its cellular localization and its putative interaction as macromolecular complexes with the cell- adhesion/signaling beta-catenin molecule and the glycogen synthase kinase 3beta (GSK-3beta) enzyme. Developmental studies have shown that PS proteins function as components in the Notch signal transduction cascade and that beta-catenin and GSK-3beta are transducers of the Wnt signaling pathway. Both pathways are thought to have an important role in brain development, and they have been connected through Dishevelled (Dvl) protein, a known transducer of the Wnt pathway.

Members of the vertebrate Wnt family have been subdivided into two functional classes according to their biological activities. Some Wnts signal through the canonical Wnt-1/wingless pathway by stabilizing cytoplasmic beta-catenin. By contrast other Wnts stimulate intracellular Ca2+ release and activate two kinases, CamKII and PKC, in a G-protein-dependent manner. Moreover, putative Wnt receptors belonging to the Frizzled gene family have been identified that preferentially couple to the two prospective pathways in the absence of ectopic Wnt ligand and that might account for the signaling specificity of the Wnt pathways. As Ca2+ release was the first described feature of the noncanonical pathway, and as Ca2+ probably plays a key role in the activation of CamKII and PKC, Kuhl M, et al., (Trends Genet July 2000 ;16(7):279-83) have named this Wnt pathway the Wnt/Ca2+ pathway.

Many constituents of Wnt signaling pathways are expressed in the developing and mature nervous systems. Recent work has shown that Wnt signaling controls initial formation of the neural plate and many subsequent patterning decisions in the embryonic nervous system, including formation of the neural crest. Wnt signaling continues to be important at later stages of development. Wnts have been shown to regulate the anatomy of the neuronal cytoskeleton and the differentiation of synapses in the cerebellum. Wnt signaling has been demonstrated to regulate apoptosis and may participate in degenerative processes leading to cell death in the aging brain.

Recent genetic studies have shown that the signalling factor Wnt3a is required for formation of the hippocampus; the developmental consequences of Wnt signalling in the hippocampus are mediated by multiple HMG-box transcription factors, with LEF-1 being required just for formation of the dentate gyrus.

Wnt-1 was first identified as a protooncogene activated by viral insertion in mouse mammary tumors. Transgenic expression of this gene using a mouse mammary tumor virus LTR enhancer causes extensive ductal hyperplasia early in life and mammary adenocarcinomas in approximately 50% of the female transgenic (TG) mice by 6 months of age. Metastasis to the lung and proximal lymph nodes is rare at the time tumors are detected but frequent after the removal of the primary neoplasm. The potent mitogenic effect mediated by Wnt-1 expression does not require estrogen stimulation; tumors form after an increased latency in estrogen receptor alpha-null mice. Several genetic lesions, including inactivation of p53 and over-expression of Fgf-3, collaborate with Wnt-1 in leading to mammary tumors, but loss of Sky and inactivation of one allele of Rb do not affect the rate of tumor formation in Wnt-1 TG mice.

Communication between cells is often mediated by secreted signaling molecules that bind cell surface receptors and modulate the activity of specific intracellular effectors. The Wnt family of secreted glycoproteins is one group of signaling molecules that has been shown to control a variety of developmental processes including cell fate specification, cell proliferation, cell polarity and cell migration. In addition, mis-regulation of Wnt signaling can cause developmental defects and is implicated in the genesis of several human cancers. The importance of Wnt signaling in development and in clinical pathologies is underscored by the large number of primary research papers examining various aspects of Wnt signaling that have been published in the past several years.

Reproductive tract development and function is regulated by circulating steroid hormones. In the mammalian female reproductive tract, estrogenic compounds direct many aspects of cytodifferentiation including uterine gland formation, smooth muscle morphology, and epithelial differentiation. While it is clear that these hormones act through their cognate nuclear receptors, it is less clear what signaling events follow hormonal stimulation that govern cytodifferentiation. Recent advances in molecular embryology and cancer cell biology have identified the Wnt family of secreted signaling molecules. Discussed here are recent advances that point to a definitive role during uterine development and adult function for one member of the Wnt gene family, Wnt-7a. In addition, recent data is reviewed that implicates Wnt-7a deregulation in response to pre-natal exposure to the synthetic estrogenic compound, DES. These advances point to an important role for the Wnt gene family in various reproductive tract pathologies including cancer.

Holoprosencephaly (HPE) is the most common developmental defect of the forebrain in humans. Several distinct human genes for holoprosencephaly have now been identified. They include Sonic hedgehog (SHH), ZIC2, and SIX3. Many additional genes involved in forebrain development are rapidly being cloned and characterized in model vertebrate organisms. These include Patched (Ptc), Smoothened (Smo), cubitus interuptus (ci)/Gli, wingless (wg/Wnt, decapentaplegic (dpp)/BMP, Hedgehog interacting protein (Hip), nodal, Smads, One-eyed pinhead (Oep), and TG-Interacting Factor (TGIF). However, further analysis is needed before their roles in HPE can be established.

Female reproductive hormones control mammary gland morphogenesis. In the absence of the progesterone receptor (PR) from the mammary epithelium, ductal side-branching fails to occur. Brisken C, et al. (Genes Dev March 2000 15;14(6):650-4) overcame this defect by ectopic expression of the protooncogene Wnt-1. Transplantation of mammary epithelia from Wnt-4(−)/(−) mice shows that Wnt-4 has an essential role in side-branching early in pregnancy. PR and Wnt-4 mRNAs colocalize to the luminal compartment of the ductal epithelium. Progesterone induces Wnt-4 in mammary epithelial cells and is required for increased Wnt-4 expression during pregnancy. Thus, Wnt signaling is essential in mediating progesterone function during mammary gland morphogenesis.

Synapse formation requires changes in cell morphology and the upregulation and localization of synaptic proteins. In the cerebellum, mossy fibers undergo extensive remodeling as they contact several granule cells and form complex, multisynaptic glomerular rosettes. Hall A C, et al., (Cell March 2000 3;100(5):525-35) showed that granule cells secrete factors that induce axon and growth cone remodeling in mossy fibers. This effect is blocked by the WNT antagonist, sFRP-1, and mimicked by WNT-7a, which is expressed by granule cells. WNT-7a also induces synapsin I clustering at remodeled areas of mossy fibers, a preliminary step in synaptogenesis. Wnt-7a mutant mice show a delay in the morphological maturation of glomerular rosettes and in the accumulation of synapsin I. We propose that WNT-7a can function as a synaptogenic factor.

Estrogens have important functions in mammary gland development and carcinogenesis. To better define these roles, Bocchinfuso W P, et al., (Cancer Res April 1999 15;59(8):1869-76) have used two previously characterized lines of genetically altered mice: estrogen receptor-alpha (ER alpha) knockout (ERKO) mice, which lack the gene encoding ER alpha, and mouse mammary virus tumor (MMTV)-Wnt-1 transgenic mice (Wnt-1 TG), which develop mammary hyperplasia and neoplasia due to ectopic production of the Wnt-1 secretory glycoprotein. Bocchinfuso W P, et al. have crossed these lines to ascertain the effects of ER alpha deficiency on mammary gland development and carcinogenesis in mice expressing the Wnt-1 transgene. Introduction of the Wnt-1 transgene into the ERKO background stimulates proliferation of alveolar-like epithelium, indicating that Wnt-1 protein can promote mitogenesis in the absence of an ER alpha-mediated response. The hyperplastic glandular tissue remains confined to the nipple region, implying that the requirement for ER alpha in ductal expansion is not overcome by ectopic Wnt-1. Tumors were detected in virgin ERKO females expressing the Wnt-1 transgene at an average age (48 weeks) that is twice that seen in virgin Wnt-1 TG mice (24 weeks) competent to produce ER alpha. Prepubertal ovariectomy of Wnt-1 TG mice also extended tumor latency to 42 weeks. However, pregnancy did not appear to accelerate the appearance of tumors in Wnt-1 TG mice, and tumor growth rates were not measurably affected by late ovariectomy. Small hyperplastic mammary glands were observed in Wnt-1 TG males, regardless of ER alpha gene status; the glands were similar in appearance to those found in ERKO/Wnt-1 TG females. Mammary tumors also occurred in Wnt-1 TG males; latency tended to be longer in the heterozygous ER alpha and ERKO males (86 to 100 weeks) than in wild-type ER alpha mice (ca. 75 weeks). Bocchinfuso W P, et al. concluded that ectopic expression of the Wnt-1 proto-oncogene can induce mammary hyperplasia and tumorigenesis in the absence of ER alpha in female and male mice. The delayed time of tumor appearance may depend on the number of cells at risk of secondary events in the hyperplastic glands, on the carcinogenesis-promoting effects of ER alpha signaling, or on both.

Wnt-1 and Wnt-3a proto-oncogenes have been implicated in the development of midbrain and hindbrain structures. Evidence for such a role has been derived from in situ hybridization studies showing Wnt-1 and -3a expression in developing cranial and spinal cord regions and from studies of mutant mice whose Wnt-1 genes have undergone targeted disruption by homologous recombination. Wnt-1 null mutants exhibit cranial defects but no spinal cord abnormalities, despite expression of the gene in these regions. The absence of spinal cord abnormalities is thought to be due to a functional compensation of the Wnt-1 deficiency by related genes, a problem that has complicated the analysis of null mutants of other developmental genes as well. Augustine K, et al., (Dev Genet 1993;14(6):500-20) describe the attenuation of Wnt-1 expression using antisense oligonucleotide inhibition in mouse embryos grown in culture. Augustine K, et al. induced similar mid-and hindbrain abnormalities as those seen in the Wnt-1 null mutant mice. Attenuation of Wnt-1 expression was also associated with cardiomegaly resulting in hemostasis. These findings are consistent with the possibility that a subset of Wnt-1 expressing cells include neural crest cells known to contribute to septation of the truncus arteriosus and to formation of the visceral arches. Antisense knockout of Wnt-3a, a gene structurally related to Wnt-1, targeted the forebrain and midbrain region, which were hypoplastic and failed to expand, and the spinal cord, which exhibited lateral outpocketings at the level of the forelimb buds. Dual antisense knockouts of Wnt-1 and Wnt-3a targeted all brain regions leading to incomplete closure of the cranial neural folds, and an increase in the number and severity of outpocketings along the spinal cord, suggesting that these genes complement one another to produce normal patterning of the spinal cord. The short time required to assess the mutant phenotype (2 days) and the need for limited sequence information of the target gene (20-25 nucleotides) make this antisense oligonucleotide/whole embryo culture system ideal for testing the importance of specific genes and their interactions in murine embryonic development.

Wnt-1 (previously known as int-1) is a proto-oncogene induced by the integration of the mouse mammary tumor virus. It is thought to play a role in intercellular communication and seems to be a signalling molecule important in the development of the central nervous system (CNS). The sequence of wnt-1 is highly conserved in mammals, fish, and amphibians. Wnt-1 is a member of a large family of related proteins that are all thought to be developmental regulators. These proteins are known as wnt-2 (also known as irp), wnt-3 up to wnt-15. At least four members of this family are present in Drosophila. One of them, wingless (wg), is implicated in segmentation polarity. All these proteins share the following features characteristics of secretory proteins, a signal peptide, several potential N-glycosylation sites and 22 conserved cysteines that are probably involved in disulfide bonds. The Wnt proteins seem to adhere to the plasma membrane of the secreting cells and are therefore likely tosignal over only few cell diameters.

The disclosed NOV7 nucleic acid of the invention encoding a Wnt-15-like protein includes the nucleic acid whose sequence is provided in Table 7A or a fragment thereof. The invention also includes a mutant or variant nucleic acid any of whose bases may be changed from the corresponding base shown in Table 7A while still encoding a protein that maintains its Wnt-15-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 32 percent of the bases may be so changed.

The disclosed NOV7 protein of the invention includes the Wnt-15-like protein whose sequence is provided in Table 7B. The invention also includes a mutant or variant protein any of whose residues may be changed from the corresponding residue shown in Table 7B while still encoding a protein that maintains its Wnt-15-like activities and physiological functions, or a functional fragment thereof. In the mutant or variant protein, up to about 38 percent of the residues may be so changed.

The above defined information for this invention suggests that these Wnt-15-like proteins (NOV7) may function as a member of a “Wnt-15 family”. Therefore, the NOV7 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The nucleic acids and proteins of NOV7 are useful in Von Hippel-Lindau (VHL) syndrome, Alzheimer's disease, stroke, tuberous sclerosis, hypercalceimia, Parkinson's disease, Huntington's disease, cerebral palsy, epilepsy, Lesch-Nyhan syndrome, multiple sclerosis, ataxia-telangiectasia, leukodystrophies, behavioral disorders, addiction, anxiety, pain, neurodegeneration, cancer, developmental defects, and/or other pathologies and disorders. The novel NOV7 nucleic acid encoding NOV7 protein,, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods.

NOV7 nucleic acids and polypeptides are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. For example the disclosed NOV7 protein have multiple hydrophilic regions, each of which can be used as an immunogen. In one embodiment, contemplated NOV7 epitope is from about amino acids 25 to 60. In other embodiments, NOV7 epitope is from about amino acids 65 to 80, from about amino acids 110 to 140, from about amino acids 145 to 180, from about amino acids 190 to 220, from about amino acids 230 to 270, or from about amino acids 280 to 290. This novel protein also has value in development of powerful assay system for functional analysis of various human disorders, which will help in understanding of pathology of the disease and development of new drug targets for various disorders.

NOV8

A disclosed NOV8 nucleic acid of 1085 nucleotides (also referred to 88091010_EXT) encoding a novel Wnt-14-like protein is shown in Table 8A. An open reading frame was identified beginning with an ATG initiation codon at nucleotides 13-15 and ending with a TGA codon at nucleotides 1078-1080. In Table 8A, the 5′ and 3′ untranslated regions are underlined and the start and stop codons are in bold letters.

TABLE 8A
NOV8 Nucleotide Sequence

(SEQ ID NO:21)

TAGTGAGCCGAGATGGCACTACTATATTCCAGCTTGGGTGTGGTTGTGTGCACCTGTAGTCCTAGTTACTT
TGGACTGACGGGCAGCGAGCCCCTGACCATCCTCCCGCTGACCCTGGAGCCAGAGGCGGCTGCCCAGGCGC
ACTACAAGGCCTGCGACCGGCTGAAGCTGGAGCGGAAGCAGCGGCGCATGTGCCGCCGGGACCCGGGCGTG
GCAGAGACGCTGGTGGAGGCCGTGAGCATGAGTGCGCTCGAGTGCCAGTTCCAGTTCCGCTTTGAGCGCTG
GAACTGCACGCTGGAGGGCCGCTACCGGGCCAGCCTGCTCAAGCGAGGTTTCAAGGAGACTGCCTTCCTCT
ATGCCATCTCCTCGGCTGGCCTGACGCACGCACTGGCCAAGGCGTGCAGCGCGGGCCGCATGGAGCGCTGT
ACCTGCGATGAGGCACCCGACCTGGAGAACCGTGAGGGCTGGAAGTGGGGTGGCTGTAGCGAGGACATCGA
GTTTGGTGGGATGGTGTCTCGGGAGTTCGCCGACGCCCGGGAGAACCGGCCAGATGCCCGCTCAGCCATGA
ACCGCCACAACAACGAGGCTGGGCGCCAGGTGATCAAGGCTGGGGTGGAGACCACCTGCAAGTGCCACGGC
GTGTCAGGCTCATGCACGGTGCGGACCTGCTGGCGGCAGTTGGCGCCTTTCCATGAGGTGGGCAAGCATCT
GAAGCACAAGTATGAGTCGGCACTCAAGGTGGGCAGCACCACCAATGAAGCTGCCGGCGAGGCAGGTGCCA
TCTCCCCACCACGGGGCCGTGCCTCGGGGGCAGGTGGCAGCGACCCGCTGCCCCGCACTCCAGAGCTGGTG
CCGTGAGAAGAACTGCGAGAGCATCTGCTGTGGCCGCGGCCATAACACACAGAGCCGGGTGGTGACAAGGC
CCTGCCAGTGCCAGGTGCGTTGGTGCTGCTATGTGGAGTGCAGGCAGTGCACGCAGCGTGAGGAGGTCTAC
ACCTGCAAGGGCTGAGTTCC

The disclosed NOV8 nucleic acid sequence, localized to chromosome 1, has 560 of 725 bases (77%) identical to a gb:GENBANK-ID:AF031168|acc:AF031168.1 mRNA from Gallus gallus (Gallus gallus Wnt-14 protein (Wnt-14) mRNA, complete cds (E=5.2e⁻¹¹⁵)

A disclosed NOV8 polypeptide (SEQ ID NO: 22) encoded by SEQ ID NO: 21 is 355 amino acid residues and is presented using the one-letter amino acid code in Table 8B. Signal P, Psort and/or Hydropathy results predict that NOV8 has a signal peptide and is likely to be localized extracellularly with a certainty of 0.3700. In other embodiments, NOV8 is also likely to be localized to the enoplasmic reticulum (membrane) with a certainty of 0.1000, to the endoplasmic reticulum (lumen) with a certainty of 0.1000, or the lysosome (lumen) with a certainty of 0.1000. The most likely cleavage site for a NOV8 peptide is between amino acids 15 and 16, at: CTC-SP.

TABLE 8B
Encoded NOV8 protein sequence.

(SEQ ID NO:22)

MALLYSSLGVVVCTCSPSYFGLTGSEPLTILPLTLEPEAAAQAHYKACDR
LKLERKQRRMCRRDPGVAETLVEAVSMSALECQFQFRFERWNCTLEGRYR
ASLLKRGFKETAFLYAISSAGLTHALAKACSAGRMERCTCDEAPDLENRE
GWKWGGCSEDIEFGGMVSREFADARENRPDARSAMNRHNNEAGRQVIKAG
VETTCKCHGVSGSCTVRTCWRQLAPFHEVGKHLKHKYESALKVGSTTNEA
AGEAGAISPPRGRASGAGGSDPLPRTPELVHLDDSPSFCLAGRFSPGTAG
RRCHREKNCESICCGRGHNTQSRVVTRPCQCQVRWCCYVECRQCTQREEV
YTCKG

The disclosed NOV8 amino acid sequence has 270 of 354 amino acid residues (76%) identical to, and 310 of 354 amino acid residues (87%) similar to, the 354 amino acid residue ptnr:SWISSPROT-ACC:042280 protein from Gallus gallus (Chicken) (WNT-14 Protein Precursor (1.2e⁻¹⁵¹).

NOV8 is expressed in at least brain. This information was derived by determining the tissue sources of the sequences that were included in the invention including but not limited to SeqCalling sources, Public EST sources, Literature sources, and/or RACE sources.

In addition, the sequence is predicted to be expressed in brain because of the expression pattern of (GENBANK-ID: gb:GENBANK-ID:AF031168|acc:AF031168.1) a closely related [Gallus gallus Wnt-14 protein (Wnt-14) mRNA, complete cds].

NOV8 also has homology to the amino acid sequence shown in the BLASTP data listed in Table 8C.

TABLE 8C
BLAST results for NOV8

| Gene Index/ | | Length | Identity | Positives | |
|---|---|---|---|---|---|
| Identifier | Protein/Organism | (aa) | (%) | (%) | Expect |
| gi\|15082261\|ref\|NP_{—} | wingless-type | 365 | 306/340 | 321/340 | e−167 |
| 003386.1\| | MMTV integration | | (90%) | (94%) | |
| (NM_003395) | site family, | | | | |
| | member 14 [Homo | | | | |
| | sapiens] | | | | |
| gi\|3915306\|sp\|O42280 | WNT-14 PROTEIN | 354 | 270/357 | 310/357 | e−142 |
| \|WN14_CHICK | PRECURSOR | | (75%) | (86%) | |
| gi\|16303264\|dbj\| | WNT14B [Homo | 357 | 193/339 | 244/339 | e−100 |
| BAB70499.1\| | sapiens] | | (56%) | (71%) | |
| (AB063483) | | | | | |
| gi\|7106447\|ref\|NP_{—} | wingless-related | 352 | 141/311 | 179/311 | 2e−62 |
| 033548.1\| (NM_{—} | MMTV integration | | (45%) | (57%) | |
| 009522) | site 3A [Mus | | | | |
| | musculus] | | | | |
| gi\|5821261\|dbj\| | Wnt-3a [Gallus | 376 | 139/311 | 179/311 | 3e−62 |
| BAA83743.1\| | gallus] | | (44%) | (56%) | |
| (AB024080) | | | | | |

The homology of these sequences is shown graphically in the ClustalW analysis shown in Table 8D.

[image]

[image]

Tables 8E and 8F list the domain descriptions from DOMAIN analysis results against NOV8. This indicates that the NOV8 sequence has properties similar to those of other proteins known to contain this domain.

TABLE 8E
Domain Analysis of NOV8

gnl|Pfam|pfam0010, wnt, wnt family. (SEQ ID NO:104)
CD-Length = 313 residues, 99.7% aligned
Score = 313 bits (801), Expect = le−86

| | | | |
|---|---|---|---|
| NOV 7: | 48 | CDRLK-LERKQRRMCRRDPGVAETLVEAVSMSALECQFQFRFERWNCTLEGRYRASL--- | 103 |
| | | \| \| \| +\|\|++\|\|\|+\| \| ++ \| ++ \|\|\| \|\|\| \|\|\|\|+ \| \| | |
| Sbjct: | 2 | CRSLPGLSPRQRQLCRRNPDVMASVSEGAQLAIQECQHQFRGRRWNCSTLDRLRVVFGKV | 61 |
| NOV 7: | 104 | LKRGFKETAFLYAISSAGLTHALAKACSAGRMERCTCDE-APDLENREGWKWGGCSEDIE | 162 |
| | | \|\|+\| +\|\|\|\|+\|\|\|\|\|\|\|+ \|\|+ +\|\|\| \| +\| \| \|\| + \|+\|\|\|\|\|+++\| | |
| Sbjct: | 62 | LKKGTRETAFVYAISSAGVAHAVTRACSEGELESCGCDYKKGPGGPQGSWQWGGCSDNVE | 121 |
| NOV 7: | 163 | FGGMVSREFADARENRPDARSAMNRHNNEAGRQVIKAGVETTCKCHGVSGSCTVRTCWRQ | 222 |
| | | \|\| \|\|\|\| \|\|\|\| \|\|\|\| \|\| \|\|\|\|\|\|\|+ +\|+ + \|\|\|\|\|\|\|\|\|\|+++\|\|\| | |
| Sbjct: | 122 | FGIRFSREFVDARERERDARSLMNLHNNEAGRKAVKSHMRRECKCHGVSGSCSMKTCWLS | 181 |
| NOV 7: | 223 | LAPFHEVGKHLKHKYESALKV-GSTTNEAAGEAGAISPPRCRASOAGGSDPLPRTPELVH | 281 |
| | | \| \| \|\| \|\| \|\|+ \|++\| + \| \| + \| \|\| \|\|+ | |
| Sbjct: | 182 | LPDFRAVGDALKDKYDGAIRVEPNKRGMGQGSAPRLVAKNPRFKPPTRSD-------LVY | 234 |
| NOV 7: | 282 | LDDSPSFCL--AGRFSPGTAGRRC----HREKNCESICCGRGHNTQSRVVTRPCQCQVRW | 335 |
| | | \|+\|\|\| +\| \| \|\| \|\| \| \|\| +\|\|\|\|\|+\|\|\| \| \| \|+ \| | |
| Shjct: | 235 | LEDSPDYCERDRSTGSLGTQGRVCNKTSKGLDGCELLCCGRGYNTQQVERTEKCNCKFHW | 294 |
| NOV 7: | 336 | CCYVECRQCTQREEVYTCK 354 | |
| | | \|\|\|\|+\| +\| + \|\|+\|\|\| | |
| Sbjct: | 295 | CCYVKCEECQEVVEVHTCK 313 | |

TABLE 8F
Domain Analysis of NOV8

gnl|Smart|smart00097, WNT1, found in Wnt-1 (SEQ ID NO:105)
CD-Length = 304 residues, 98.7% aligned
Score = 292 bits (748), Expect = 2e−80

| | | | |
|---|---|---|---|
| NOV 7: | 53 | LERKQRRMCRRDPGVAETLVEAVSMSALECQFQFRFERWNCTLEGRYRA--SLLKRGFKE | 110 |
| | | \| \|+\|\|++\|\| +\| \| ++ \| \|\|\| \|\|\|\| \|\|\|\|+ \| +\|++\| +\| | |
| Sbjct: | 5 | LSRRQRQLCRANPDVMASVAEGAQEGIEECQHQFRFRRWNCSTAGLASIFOKVLRQGTRE | 64 |
| NOV 7: | 111 | TAFLYAISSAGLTHALAKACSAGRMERCTCDEAPDLENREGWKWGGCSEDIEFGGMVSRE | 170 |
| | | \|\|\|+\|\|\|\|\|\|\|+ \|\|+ +\|\|\| \| ++ \| \|\| + \|\|+\|\|\|\|\|++\|+\|\| \|\|\| | |
| Sbjct: | 65 | TAFVYAISSAGVAHAVTRACSQGELDSCGCDYSKRGSGGRGWEWGGCSDNIDFGIGFSRE | 124 |
| NOV 7: | 171 | FADARER-PDARSAMNRHNNEAGRQVIKAGVETTCKCHGVSGSCTVRTCWRQLAPFHEV | 229 |
| | | \| \|\|\|\| \| \|\|\|+ \|\| \|\|\|\|\|\| +\| ++ \|\|\|\|\|\|\|\|\|\|+\|+\|\|\| \|\| \| \|+ | |
| Sbjct: | 125 | FVDARERRGSDARALMNLHNNEAGRLAVKKTMXRECKCHOVSGSCSVKTCWLQLPEFREI | 184 |
| NOV 7: | 230 | CKHLRHKYESALKVGSTTNEAAGEAGAISPPRGRASGAGGSDPLPRTPELVHLDDSPSFC | 289 |
| | | \| +\|\| \|\|+ \| +\| \| \| +\|\|+\|+ \|\| \|\| | |
| Sbjct: | 185 | GDYLKEKYDCASEVVLD-----------KRGTRGLVPANRDFKPPTNTDLVYLESSPDFC | 233 |
| NOV 7: | 290 | LAGRF--SPCTAGRRCHREKN----CESICCGRGHNTQSRVVTRPCQCQVRWCCYVECRQ | 343 |
| | | \| \|\| \|\| \|++ \|+ +\|\|\|\|\|+\|\|+ \| \| \|+ \|\|\|\|\|+\|+\| | |
| Sbjct: | 234 | EKNPKTGSLGTQGRVCNKTSKCLDOCDLLCCORGYNTEHVEVVERCNCKFHWCCYVKCKQ | 293 |
| NOV 7: | 344 | CTQREEVYTCK 354 | |
| | | \| +\| \| +\|\|\| | |
| Sbjct: | 294 | CRERVEKHTCK 304 | |

Wnt proteins constitute a large family of molecules involved in cell proliferation, cell differentiation and embryonic patterning. They are known to interact with the Frizzled family of receptors to activate two main intracellular signaling pathways regulating intracellular calcium levels and gene transcription. Early studies on Wnts implicated them in cell proliferation and tumorigenesis, which have been borne out by recent work using transgenic and null mutant mice. Wnts are involved in processes involved in mammary gland development and cancer. Recent studies have demonstrated that these molecules are critical to organogenesis of several systems, such as the kidney and brain. Wnts regulate the early development, i.e. neural induction, and their role persists in later stages of development as well as in the mature organ. An example of this is seen in the brain, where the loss of certain Wnts leads to the absence of critical regions of the brain, e.g. the hippocampus, involved in learning and memory, or the cerebellum, involved in motor function. Wnts have also been implicated in the genesis of degenerative diseases such as Alzheimer's disease. The protein encoded by the novel gene described herein may therefore play a role in cellular proliferation, differentiation, dysregulation, organogenesis and disease processes such as cancer, developmental defects etc.

Alzheimer's disease (AD) is a neurodegenerative disease with progressive dementia accompanied by three main structural changes in the brain: diffuse loss of neurons; intracellular protein deposits termed neurofibrillary tangles (NFT) and extracellular protein deposits termed amyloid or senile plaques, surrounded by dystrophic neurites. Two major hypotheses have been proposed in order to explain the molecular hallmarks of the disease: The ‘amyloid cascade’ hypothesis and the ‘neuronal cytoskeletal degeneration’ hypothesis. While the former is supported by genetic studies of the early-onset familial forms of AD (FAD), the latter revolves around the observation in vivo that cytoskeletal changes—including the abnormal phosphorylation state of the microtubule associated protein tau—may precede the deposition of senile plaques. Recent studies have suggested that the trafficking process of membrane associated proteins is modulated by the FAD-linked presenilin (PS) proteins, and that amyloid beta-peptide deposition may be initiated intracellularly, through the secretory pathway. Current hypotheses concerning presenilin function are based upon its cellular localization and its putative interaction as macromolecular complexes with the cell-adhesion/signaling beta-catenin molecule and the glycogen synthase kinase 3beta (GSK-3beta) enzyme. Developmental studies have shown that PS proteins function as components in the Notch signal transduction cascade and that beta-catenin and GSK-3beta are transducers of the Wnt signaling pathway. Both pathways are thought to have an important role in brain development, and they have been connected through Dishevelled (Dvl) protein, a known transducer of the Wnt pathway.

Members of the vertebrate Wnt family have been subdivided into two functional classes according to their biological activities. Some Wnts signal through the canonical Wnt-1/wingless pathway by stabilizing cytoplasmic beta-catenin. By contrast other Wnts stimulate intracellular Ca2+ release and activate two kinases, CamKII and PKC, in a G-protein-dependent manner. Moreover, putative Wnt receptors belonging to the Frizzled gene family have been identified that preferentially couple to the two prospective pathways in the absence of ectopic Wnt ligand and that might account for the signaling specificity of the Wnt pathways. As Ca2+ release was the first described feature of the noncanonical pathway, and as Ca2+ probably plays a key role in the activation of CamKII and PKC, Kuhl M, et al., (Trends Genet July 2000 ;16(7):279-83) have named this Wnt pathway the Wnt/Ca2+ pathway.

Many constituents of Wnt signaling pathways are expressed in the developing and mature nervous systems. Recent work has shown that Wnt signaling controls initial formation of the neural plate and many subsequent patterning decisions in the embryonic nervous system, including formation of the neural crest. Wnt signaling continues to be important at later stages of development. Wnts have been shown to regulate the anatomy of the neuronal cytoskeleton and the differentiation of synapses in the cerebellum. Wnt signaling has been demonstrated to regulate apoptosis and may participate in degenerative processes leading to cell death in the aging brain.

Recent genetic studies have shown that the signalling factor Wnt3a is required for formation of the hippocampus; the developmental consequences of Wnt signalling in the hippocampus are mediated by multiple HMG-box transcription factors, with LEF-1 being required just for formation of the dentate gyrus.

Wnt-1 was first identified as a protooncogene activated by viral insertion in mouse mammary tumors. Transgenic expression of this gene using a mouse mammary tumor virus LTR enhancer causes extensive ductal hyperplasia early in life and mammary adenocarcinomas in approximately 50% of the female transgenic (TG) mice by 6 months of age. Metastasis to the lung and proximal lymph nodes is rare at the time tumors are detected but frequent after the removal of the primary neoplasm. The potent mitogenic effect mediated by Wnt-1 expression does not require estrogen stimulation; tumors form after an increased latency in estrogen receptor alpha-null mice. Several genetic lesions, including inactivation of p53 and over-expression of Fgf-3, collaborate with Wnt-1 in leading to mammary tumors, but loss of Sky and inactivation of one allele of Rb do not affect the rate of tumor formation in Wnt-1 TG mice.

Communication between cells is often mediated by secreted signaling molecules that bind cell surface receptors and modulate the activity of specific intracellular effectors. The Wnt family of secreted glycoproteins is one group of signaling molecules that has been shown to control a variety of developmental processes including cell fate specification, cell proliferation, cell polarity and cell migration. In addition, mis-regulation of Wnt signaling can cause developmental defects and is implicated in the genesis of several human cancers. The importance of Wnt signaling in development and in clinical pathologies is underscored by the large number of primary research papers examining various aspects of Wnt signaling that have been published in the past several years.

Reproductive tract development and function is regulated by circulating steroid hormones. In the mammalian female reproductive tract, estrogenic compounds direct many aspects of cytodifferentiation including uterine gland formation, smooth muscle morphology, and epithelial differentiation. While it is clear that these hormones act through their cognate nuclear receptors, it is less clear what signaling events follow hormonal stimulation that govern cytodifferentiation. Recent advances in molecular embryology and cancer cell biology have identified the Wnt family of secreted signaling molecules. Discussed here are recent advances that point to a definitive role during uterine development and adult function for one member of the Wnt gene family, Wnt-7a. In addition, recent data is reviewed that implicates Wnt-7a deregulation in response to pre-natal exposure to the synthetic estrogenic compound, DES. These advances point to an important role for the Wnt gene family in various reproductive tract pathologies including cancer.

Holoprosencephaly (HPE) is the most common developmental defect of the forebrain in humans. Several distinct human genes for holoprosencephaly have now been identified. They include Sonic hedgehog (SHH), ZIC2, and SIX3. Many additional genes involved in forebrain development are rapidly being cloned and characterized in model vertebrate organisms. These include Patched (Ptc), Smoothened (Smo), cubitus interuptus (ci)/Gli, wingless (wg/Wnt, decapentaplegic (dpp)/BMP, Hedgehog interacting protein (Hip), nodal, Smads, One-eyed pinhead (Oep), and TG-Interacting Factor (TGIF). However, further analysis is needed before their roles in HPE can be established.

Female reproductive hormones control mammary gland morphogenesis. In the absence of the progesterone receptor (PR) from the mammary epithelium, ductal side-branching fails to occur. Brisken C, et al. (Genes Dev March 2000 15;14(6):650-4) overcame this defect by ectopic expression of the protooncogene Wnt-1. Transplantation of mammary epithelia from Wnt-4(−)/(−) mice shows that Wnt-4 has an essential role in side-branching early in pregnancy. PR and Wnt-4 mRNAs colocalize to the luminal compartment of the ductal epithelium. Progesterone induces Wnt-4 in mammary epithelial cells and is required for increased Wnt-4 expression during pregnancy. Thus, Wnt signaling is essential in mediating progesterone function during mammary gland morphogenesis.

Synapse formation requires changes in cell morphology and the upregulation and localization of synaptic proteins. In the cerebellum, mossy fibers undergo extensive remodeling as they contact several granule cells and form complex, multisynaptic glomerular rosettes. Hall A C, et al., (Cell March 3, 2000; 100(5):525-35) showed that granule cells secrete factors that induce axon and growth cone remodeling in mossy fibers. This effect is blocked by the WNT antagonist, sFRP-1, and mimicked by WNT-7a, which is expressed by granule cells. WNT-7a also induces synapsin I clustering at remodeled areas of mossy fibers, a preliminary step in synaptogenesis. Wnt-7a mutant mice show a delay in the morphological maturation of glomerular rosettes and in the accumulation of synapsin I. We propose that WNT-7a can function as a synaptogenic factor.

Estrogens have important functions in mammary gland development and carcinogenesis. To better define these roles, Bocchinfuso W P, et al., (Cancer Res April 1999 15;59(8): 1869-76) have used two previously characterized lines of genetically altered mice: estrogen receptor-alpha (ER alpha) knockout (ERKO) mice, which lack the gene encoding ER alpha, and mouse mammary virus tumor (MMTV)-Wnt-1 transgenic mice (Wnt-1 TG), which develop mammary hyperplasia and neoplasia due to ectopic production of the Wnt-1 secretory glycoprotein. Bocchinfuso W P, et al. have crossed these lines to ascertain the effects of ER alpha deficiency on mammary gland development and carcinogenesis in mice expressing the Wnt-1 transgene. Introduction of the Wnt-i transgene into the ERKO background stimulates proliferation of alveolar-like epithelium, indicating that Wnt-1 protein can promote mitogenesis in the absence of an ER alpha-mediated response. The hyperplastic glandular tissue remains confined to the nipple region, implying that the requirement for ER alpha in ductal expansion is not overcome by ectopic Wnt-1. Tumors were detected in virgin ERKO females expressing the Wnt-1 transgene at an average age (48 weeks) that is twice that seen in virgin Wnt-1 TG mice (24 weeks) competent to produce ER alpha. Prepubertal ovariectomy of Wnt-1 TG mice also extended tumor latency to 42 weeks. However, pregnancy did not appear to accelerate the appearance of tumors in Wnt-1 TG mice, and tumor growth rates were not measurably affected by late ovariectomy. Small hyperplastic mammary glands were observed in Wnt-1 TG males, regardless of ER alpha gene status; the glands were similar in appearance to those found in ERKO/Wnt-1 TG females. Mammary tumors also occurred in Wnt-1 TG males; latency tended to be longer in the heterozygous ER alpha and ERKO males (86 to 100 weeks) than in wild-type ER alpha mice (ca. 75 weeks). Bocchinfuso W P, et al. concluded that ectopic expression of the Wnt-1 proto-oncogene can induce mammary hyperplasia and tumorigenesis in the absence of ER alpha in female and male mice. The delayed time of tumor appearance may depend on the number of cells at risk of secondary events in the hyperplastic glands, on the carcinogenesis-promoting effects of ER alpha signaling, or on both.

Wnt-1 and Wnt-3a proto-oncogenes have been implicated in the development of midbrain and hindbrain structures. Evidence for such a role has been derived from in situ hybridization studies showing Wnt-1 and -3a expression in developing cranial and spinal cord regions and from studies of mutant mice whose Wnt-1 genes have undergone targeted disruption by homologous recombination. Wnt-1 null mutants exhibit cranial defects but no spinal cord abnormalities, despite expression of the gene in these regions. The absence of spinal cord abnormalities is thought to be due to a functional compensation of the Wnt-1 deficiency by related genes, a problem that has complicated the analysis of null mutants of other developmental genes as well. Augustine K, et al., (Dev Genet 1993; 14(6):500-20) describe the attenuation of Wnt-1 expression using antisense oligonucleotide inhibition in mouse embryos grown in culture. Augustine K, et al. induced similar mid-and hindbrain abnormalities as those seen in the Wnt-1 null mutant mice. Attenuation of Wnt-1 expression was also associated with cardiomegaly resulting in hemostasis. These findings are consistent with the possibility that a subset of Wnt-1 expressing cells include neural crest cells known to contribute to septation of the truncus arteriosus and to formation of the visceral arches. Antisense knockout of Wnt-3a, a gene structurally related to Wnt-1, targeted the forebrain and midbrain region, which were hypoplastic and failed to expand, and the spinal cord, which exhibited lateral outpocketings at the level of the forelimb buds. Dual antisense knockouts of Wnt-1 and Wnt-3a targeted all brain regions leading to incomplete closure of the cranial neural folds, and an increase in the number and severity of outpocketings along the spinal cord, suggesting that these genes complement one another to produce normal patterning of the spinal cord. The short time required to assess the mutant phenotype (2 days) and the need for limited sequence information of the target gene (20-25 nucleotides) make this antisense oligonucleotide/whole embryo culture system ideal for testing the importance of specific genes and their interactions in murine embryonic development.

Wnt-1 (previously known as int-1) is a proto-oncogene induced by the integration of the mouse mammary tumor virus. It is thought to play a role in intercellular communication and seems to be a signalling molecule important in the development of the central nervous system (CNS). The sequence of wnt-1 is highly conserved in mammals, fish, and amphibians. Wnt-1 is a member of a large family of related proteins that are all thought to be developmental regulators. These proteins are known as wnt-2 (also known as irp), wnt-3 up to wnt-15. At least four members of this family are present in Drosophila. One of them, wingless (wg), is implicated in segmentation polarity. All these proteins share the following features characteristics of secretory proteins, a signal peptide, several potential N-glycosylation sites and 22 conserved cysteines that are probably involved in disulfide bonds. The Wnt proteins seem to adhere to the plasma membrane of the secreting cells and are therefore likely tosignal over only few cell diameters.

The disclosed NOV8 nucleic acid of the invention encoding a Wnt-14-like protein includes the nucleic acid whose sequence is provided in Table 8A or a fragment thereof. The invention also includes a mutant or variant nucleic acid any of whose bases may be changed from the corresponding base shown in Table 8A while still encoding a protein that maintains its Wnt-14-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 23 percent of the bases may be so changed.

The disclosed NOV8 protein of the invention includes the Wnt-14-like protein whose sequence is provided in Table 8B. The invention also includes a mutant or variant protein any of whose residues may be changed from the corresponding residue shown in Table 8B while still encoding a protein that maintains its Wnt-14-like activities and physiological functions, or a functional fragment thereof. In the mutant or variant protein, up to about 24 percent of the residues may be so changed.

The protein similarity information, expression pattern, and map location for the Wnt-14-like protein and nucleic acid (NOV8) disclosed herein suggest that NOV8 may have important structural and/or physiological functions characteristic of the Wnt-14-like family. Therefore, the NOV8 nucleic acids and proteins of the invention are useful in potential diagnostic and therapeutic applications. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo.

The NOV8 nucleic acids and proteins of the invention are useful in potential diagnostic and therapeutic applications implicated in various diseases and disorders described below and/or other pathologies. For example, the compositions of the present invention will have efficacy for treatment of patients suffering from Von Hippel-Lindau (VHL) syndrome, Alzheimer's disease, stroke, tuberous sclerosis, hypercalceimia, Parkinson's disease, Huntington's disease, cerebral palsy, epilepsy, Lesch-Nyhan syndrome, multiple sclerosis, ataxia-telangiectasia, leukodystrophies, behavioral disorders, addiction, anxiety, pain, neurodegeneration, cancer, developmental defects, and/or other pathologies/disorders. The NOV8 nucleic acid, or fragments thereof, may further be useful in diagnostic applications, wherein the presence or amount of the nucleic acid or the protein are to be assessed.

NOV8 nucleic acids and polypeptides are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. For example the disclosed NOV8 protein have multiple hydrophilic regions, each of which can be used as an immunogen. In one embodiment, contemplated NOV8 epitope is from about amino acids 40 to 70. In another embodiment, the comtemplated NOV8 epitope is from about amino acids 80 to 110. In further embodiments, the contemplated NOV8 epitope is from about amino acids 120 to 200, from about amino acids 220 to 245, from about amino acids 250 to 280, or from about amino acids 290 to 340. This novel protein also has value in development of powerful assay system for functional analysis of various human disorders, which will help in understanding of pathology of the disease and development of new drug targets for various disorders.

NOV9

A disclosed NOV9 nucleic acid of 2037 nucleotides (also referred to as AC069250_{—}28_da1) encoding a beta-adrenergic receptor kinase-like protein is shown in Table 9A. An open reading frame was identified beginning with an ATG initiation codon at nucleotides 16-18 and ending with a TGA codon at nucleotides 2020-2022. A putative untranslated region upstream from the initiation codon and downstream from the termination codon is underlined in Table 9A. The start and stop codons are in bold letters. Single nucleotide polymorphism data for NOV9 is discussed in further detail in Example 3.

TABLE 9A
NOV9 nucleotide sequence.

(SEQ ID NO:23)

GCCGCCGCCGCCAAGATGGCGGACCTGGAGGCGGTGCTGGCCGACGTCAGCTACCTGATGGCCATGGAGAAG
AGCAAGGCCACGCCGGCCGCGCGCGCCAGCAAGAAGATACTGCTGCCCGAGCCCAGCATCCGCAGTGTCATG
CAGAAGTACCTGGAGGACCGGCGCGAGGTGACCTTTGAGAAGATCTTTTCCCAGAAGCTGGGGTACCTGCTC
TTCCGAGACTTCTGCCTGAACCACCTGCAGGAGGCCAGGCCCTTGGTGGAATTCTATGAGGAGATCAAGAAG
TACGAGAAGCTGGAGACGGAGGAGGAGCGTGTGGCCCGCAGCCGGGAGATCTTCGACTCATACATCATGAAG
GAGCTGCTGGCCTGCTCGCATCCCTTCTCGAAGAGTGCCACTGAGCATGTCCAAGGCCACCTGGGGAAGAAG
CAGGTGCCTCCGGATCTCTTCCAGCCATACATCGAAGAGATTTGTCAAAACCTCCGAGGGGACGTGTTCCAG
AAATTCATTGAGACCGATAAGTTCACACGGTTTTGCCAGTGGAAGAATGTGGAGCTCAACATCCACCTGACC
ATGAATGACTTCAGCGTGCATCGCATCATTGGGCGCGGGGGCTTTGGCGAGGTCTATGGGTGCCGGAAGGCT
GACACAGGCAAGATGTACGCCATGAAGTGCCTGGACAAAAAGCGCATCAAGATGAAGCAGGGGGAGACCCTG
GCCCTGAACGAGCGCATCATGCTCTCGCTCGTCAGCACTGGGCACTGCCCATTCATTGTCTGCATGTCATAC
GCGTTCCACACGCCAGACAAGCTCAGCTTCATCCTGGACCTCATGAACGGTGGGGACCTGCACTACCACCTC
TCCCAGCACGGGGTCTTCTCAGAGGCTGACATGCGCTTCTATGCGGCCGAGATCATCCTGGGCCTGGAGCAC
ATGCACAACCGCTTCGTGGTCTACCGGGACCTGAAGCCAGCCAACATCCTTCTGGACGAGCATGGCCACGTG
CGGATCTCGGACCTGGGCCTGGCCTGTGACTTCTCCAAGAAGAAGCCCCATGCCAGCGTGGGCACCCACGGG
TACATGGCTCCGGAGGTCCTGCAGAAGGGCGTGGCCTACGACAGCAGTGCCGACTGGTTCTCTCTGGGGTGC
ATGCTCTTCAAGTTGCTGCGGGGGCACAGCCCCTTCCGGCAGCACAAGACCAAAGACAAGCATGAGATCGAC
CGCATGACGCTGACGATGGCCGTGGAGCTGCCCGACTCCTTCTCCCCTGAACTACGCTCCCTGCTGGAGGGG
TTGCTGCAGAGGGATGTCAACCGGAGATTGGGCTGCCTCGGCCGAGGGGCTCAGGAGCTGAAAGAGAGCCCC
TTTTTCCOCTCCCTGGACTGGCAGATGGTCTTCTTGCAGAAGTACCCTCCCCCGCTGATCCCCCCACGAGGG
GAGGTGAACGCGGCCGACGCCTTCGACATTGGCTCCTTCGATGAGGAGGACACAAAAGGAATCAAGCAGGAG
GTGGCAGAGACTGTCTTCGACACCATCAACGCTGAGACAGACCGGCTGGAGGCTCGCAAGAAAGCCAAGAAC
AAGCAGCTGGGCCATGAGGAAGACTACOCCCTGGGCAAGGACTGCATCATGCATGGCTACATGTCCAAGATG
GGCAACCCCTTCCTGACCCAGTGGCAGCGGCGGTACTTCTACCTGTTCCCCAACCGCCTCGAGTGGCGGGGC
GAGCCCGAGGCCCCGCAGAGCCTGCTGACCATGGAGGAGATCCAGTCGGTGGAGGAGACGCAGATCAAGGAG
CGCAAGTGCCTGCTCCTCAAGATCCGCGGTGCGAAACAGTTCATTTTGCAGTGCGATACCGACCCTGAGCTG
GTGCAGTGGAAGAAGGAGCTGCGCGACGCCTACCGCGAGGCCCAGCAGCTGGTGCAGCGGGTGCCCAAGATG
AAGAACAAGCCGCGCTCGCCCGTGGTGGAGCTGAGCAAGGTGCCGCTGGTCCAGCGCGGCAGTGCCAACGGC
CTCTGACCCGCCCACCCGCCT

In a search of public sequence databases, the NOV9 nucleic acid sequence, located on chromsome 11 has 1546 of 1574 bases (98%) identical to a beta-adrenergic receptor kinase 1 mRNA from Homo sapiens, (GENBANK-ID: HUMBARK1A) (E=0.0). Public nucleotide databases include all GenBank databases and the GeneSeq patent database.

The disclosed NOV9 polypeptide (SEQ ID NO: 24) encoded by SEQ ID NO: 23 has 668 amino acid residues and is presented in Table 9B using the one-letter amino acid code. Signal P, Psort and/or Hydropathy results predict that NOV9 has no signal peptide and is likely to be localized in the nucleus with a certainty of 0.3000. In other embodiments, NOV9 may also be localized to the microbody (peroxisome) with acertainty of 0.1478, the mitrochondrial matrix (lumen) with a certainty of 0.1000 or in the lysosome (lumen) with a certainty of 0.1000.

TABLE 9B
Encoded NOV9 protein sequence.

(SEQ ID NO:24)

MADLEAVLADVSYLMAMEKSKATPAARASKKILLPEPSIRSVMQKYLEDRGEVTFEKIFSQKLGYLLFRDFC
LNHLEEARPLVEFYEEIKKYEKLETEEERVARSREIFDSYIMKELLACSHPFSKSATEHVQGHLGKKQVPPD
LFQPYIEEICQNLRGDVFQKFIESDKFTRFCQWKNVELNIHLTMNDFSVHRIIGRGGFGEVYGCRKADTGKM
YAMKCLDKKRIKMKQCETLALNERIMLSLVSTGDCPFIVCMSYAFHTPDKLSFILDLMNGGDLHYHLSQHGV
FSEADMRFYAAEIILGLEHMHNRFVVYRDLKPANILLDEHGHVRISDLGLACDFSKKKPHASVGTHGYMAPE
VLQKGVAYDSSADWFSLGCMLFKLLRGHSPFRQHKTKDKHEIDRMTLTMAVELPDSFSPELRSLLEGLLQRD
VNRRLGCLGRGAQEVKESPFFRSLDWQMVFLQKYPPPLIPPRGEVNAADAFDIGSFDEEDTKGIKQEVAETV
FDTINAETDRLEARKKAKNKQLGHEEDYALGKDCIMHGYMSKMGNPFLTQWQRRYFYLFPNRLEWRGEGEAP
QSLLTMEEIQSVEETQIKERKCLLLKIRGGKQFILQCDSDPELVQWKKELRDAYREAQQLVQRVPKMKNKPR
SPVVELSKVPLVQRGSANGL

A search of sequence databases reveals that the NOV9 amino acid sequence has 495 of 497 amino acid residues (99%) identical to, and 495 of 497 amino acid residues (99%) similar to, the 689 amino acid residue beta-adrenergic receptor kinase from Homo sapiens (A53791) (E=0.0). Public amino acid databases include the GenBank databases, SwissProt, PDB and PIR.

NOV9 is expressed in at least the following tissues: adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea, uterus. This information was derived by determining the tissue sources of the sequences that were included in the invention including but not limited to SeqCalling sources, Public EST sources, Literature sources, and/or RACE sources.

In addition, the sequence is predicted to be expressed in blood leukocytes because of the expression pattern of (GENBANK-ID:gb:GENBANK-ID:HUMBARK1A|acc:M80776.1) a closely related Human beta-adrenergic receptor kinase 1 mRNA, complete cds homolog in species Homo sapiens.

The disclosed NOV9 polypeptide has homology to the amino acid sequences shown in the BLASTP data listed in Table 9C.

TABLE 9C
BLAST results for NOV9

| Gene Index/ | | Length | Identity | Positives | |
|---|---|---|---|---|---|
| Identifier | Protein/Organism | (aa) | (%) | (%) | Expect |
| ptnr: pir-id: A53791 | beta-adrenergic- | 689 | 495/497 | 495/497 | 0.0 |
| | receptor kinase | | (99%) | (99%) | |
| | (EC 2.7.1.126) 1 - | | | | |
| | human | | | | |
| ptnr: SWISSPROT- | Beta-adrenergic | 689 | 494/497 | 495/497 | 0.0 |
| ACC: P25098 | receptor kinase 1 | | (99%) | (99%) | |
| | (EC 2.7.1.126) | | | | |
| ptnr: SPTREMBL- | SIMILAR TO | 687 | 490/495 | 493/495 | 0.0 |
| ACC: Q99LL8 | ADRENERGIC, BETA, | | (98%), | (99%) | |
| | RECEPTOR KINASE 1 - | | | | |
| | Mus musculus | | | | |
| ptnr: SWISSPROT- | Beta-adrenergic | 689 | 489/497 | 493/497 | 0.0 |
| ACC: P26817 | receptor kinase 1 | | (98%) | (99%) | |
| ptnr: SPTREMBL- | G PROTEIN | 689 | 490/497 | 494/497 | 0.0 |
| ACC: Q99MK8 | RECEPTOR KINASE 2 | | (98%) | (99%) | |

The homology between these and other sequences is shown graphically in the ClustalW analysis shown in Table 9D. In the ClustalW alignment of the NOV9 proteins, as well as all other ClustalW analyses herein, the black outlined amino acid residues indicate regions of conserved sequence (i.e., regions that may be required to preserve structural or functional properties), whereas non-highlighted amino acid residues are less conserved and can potentially be altered to a much broader extent without altering protein structure or function.

[image]

[image]

[image]

Tables 9E-9L list the domain descriptions from DOMAIN analysis results against NOV9. This indicates that the NOV9 sequence has properties similar to those of other proteins known to contain this domain.

TABLE 9E
Domain Analysis of NOV9

gnl|Smart|smart00220, S_TKc, Serine Threonine protein kineses,
catalytic domain; Phosphotransferases. Serine or threonine-specific
kinase subfamily. (SEQ ID NO:98)
CD-Length = 256 residues, 100.0% aligned
Score = 237 bits (604), Expect = 2e−63

| | | | |
|---|---|---|---|
| Query: | 191 | FSVHRIIGRGGFGEVYGCRKADTGKMYAMKCLDKKRIKMKQGETLALNERIMLSLVSTGD | 250 |
| | | + + ++\|+\| \|\|+\|\| \| \|\|\|+ \|+\| + \|+++\| \|+ \| \| \| +\| + \| | |
| Sbjct: | 1 | YELLEVLGKGAFGKVYLARDKKTGKLVAIKVIKKEKLKKKKRER-ILREIKILKKL---D | 56 |
| Query: | 251 | CPFIVCMSYAFHTPDKLSFILDLMIJGGDLYHLSQHGVFSEADMRFYAAEIILGLEHMHN | 310 |
| | | \| \|\| + \| \|\|\| +++ \|\|\|\| \| + \| \|\| + \|\|\|\| +\|+ \|\|++\|+ | |
| Sbjct: | 57 | HPNIVKLYDVFEDDDKLYLVMEYCECCDLFDLLKKRGRLSEDEARFYARQILSALEYLHS | 116 |
| Query: | 311 | RFVVYRDLKPANILLDEHGHVRISDLGLACDFSKKKPHAS--VGTHGYMAPEVLQKGVAY | 368 |
| | | + +++\|\|\|\|\| \|\|\|\|\| \|\|\|+++\| \|\|\| + \|\|\| \|\|\|\|\|\|\| \| \| | |
| Sbjct: | 117 | QGIIHRDLKPENILLDSDGHVKLADFGLAKQLDSGGTLLTTFVGTPEYMAPEVL-LGKGY | 175 |
| Query: | 369 | DSSADWFSLCCMLFKLLRGHSPFRQHKTKDK-HEIDRMTLTMAVELPDSFSPELRSLLEG | 427 |
| | | + \| +\|\|\| +\|++\|\| \| \|\| + \|\|\| + \|++ | |
| Sbjct: | 176 | GKAVDIWSLGVILYELLTGKPPFPGDDQLLALFKKIGKPPPPFPPPEWKISPEAKDLIKK | 235 |
| Query: | 428 | LLQRDVNRRLGCLGRGAQEVKESPFF 453 | |
| | | \|\| +\| +\|\| \|+\| \| \|\|\| | |
| Sbjct: | 236 | LLVKDPEKRL-----TAEEALEHPFF 256 | |

TABLE 9F
Domain Analysis of NOV9

gnl|Pfam|pfam00069, pkinase, Protein kinase domain. (SEQ ID NO:99)
CD-Length = 256 residues, 100.0% aligned
Score = 221 bits (562), Expect = 1e−58

| | | | |
|---|---|---|---|
| Query: | 191 | FSVHRIIGRGGFGEVYGCRKADTGKMYAMKCLDKKRIKMKQGETLALNERIMLSLVSTGD | 250 |
| | | + + +\| \| \|\|+\|\| \| \|\|\|++ \|+\| \| \|+ + \|+ \| \| +\| +\| | |
| Sbjct: | 1 | YELGEKLGSGAFGKVYKGKHKDTGEIVAIKILKKRSLSEKKKRFL--REIQILRRLS--- | 55 |
| Query: | 251 | CPFIVCMSYAFHTPDKLSFILDLMNCGDLHYHLSQHGVF-SEADMRFYAAEIILGLEHMH | 309 |
| | | \| \|\| + \| \| \| +++ \| \|\|\|\| +\| ++\|+ \|\| + + \| +\|+ \|\|\|++\| | |
| Sbjct: | 56 | HPNIVRLLGVFEEDDHLYLVMEYMEGGDLFDYLRRNGLLLSEKEAKKIALQILRGLEYLH | 115 |
| Query: | 310 | NRFVVYRDLKPANILLDEHGHVRISDLGLACDF---SKKKPEASVGTHGYMAPEVLQKGV | 366 |
| | | +\| +\|+\|\|\|\|\| \|\|\|\|\|\|+\| \|+\|+\| \|\|\| \| +\| \|\|\| \|\|\|\|\|\| +\| | |
| Sbjct: | 116 | SRGIVHRDLKPENILLDENGTVKIADFGLARKLESSSYEKLTTFVGTPEYMAPEVL-EGR | 174 |
| Query: | 367 | AYDSSADWFSLGCMLFKLLRGHSPFRQHKTKDRHEIDRMTLTMAVELPDSFSPELRSLLE | 426 |
| | | \| \| \| +\|\|\| +\|++\|\| \| \|\| ++ + + + \|\| + \| \|\|+ \|++ | |
| Sbjct: | 175 | GYSSKVDVWSLGVILYELLTGKLPFPGIDPLEELFRIKERPRLRLPLPPNCSEELKDLIK | 234 |
| Query: | 427 | GLLQRDVNRRLGCLGRGAQEVKESPFF 453 | |
| | | \| +\| +\| \|+\|+ \|+\| | |
| Sbjct: | 235 | KCLNKDPEKRP-----TAKEILNHPWF 256 | |

TABLE 9G
Domain Analysis of NOV9

gnl|Pfam|pfam00615, RGS, Regulator of G protein signaling domain. RGS
family members are GTPase-activating proteins for heterotrimeric G-
protein alpha-subunits. (SEQ ID NO:106)
CD-Length = 119 residues, 100.0% aligned
Score = 130 bits (326), Expect = 3e−3l

| | | | |
|---|---|---|---|
| Query: | 54 | TFEKIFSQKL0YLLFRDFCLNHLEEA3PLVEEYEEIKKYEKLETEEERVARSREIFDSYI | 113 |
| | | +\|\|\|+ \| +\| \|\|\|\|+\| \| +\|\|+ +++\|\|\| \| ++\| ++\|\|\|+\| +\| | |
| Sbjct: | 1 | SFEKLLKQPIGRLLFREFLETEFSE--ENLEFWLAVEEYEKTEDPDKRPDKAREIYDEFI | 58 |
| Query: | 114 | MKELLACSHPFSKSATEHVQCHLGKKQVPPDLFQPYIEEICQNLRCDVFQRFTESDKETR | 173 |
| | | \| \|\| \| +\| \| \|\|\|+ \|\| +\|\|\| \| +\|+\|\|\| \|\|\| | |
| Sbjct: | 59 | SPEAPKPEVNLDSELREHTQDNL-LKAPTKDLFEEAQREIYDLMRGDSFPRFLESDYFTR | 117 |
| Query: | 174 | FC 175 | |
| | | \| | |
| Sbjct: | 118 | FL 119 | |

TABLE 9H
Domain Analysis of NOV9

gnl|Smart|smart00219, TyrKc, Tyrosine kinase, catalytic domain;
Pbosphotransferases. Tyrosine-specific kinase subfamily. (SEQ ID NO:100)
CD-Length = 258 residues, 94.6% aligned
Score = 110 bits (275), Expect = 3e−25

| | | | |
|---|---|---|---|
| Query: | 195 | RIIGRGGFGEVYGCR---KADTGKMYAMECLDEERIKMKQGETLALNE-RIMLSLVSTGD | 250 |
| | | + +\| \| \|\|\|\|\| \| \|+\| \| \| +\| \| \| \|+\| \| \| | |
| Sbjct: | 5 | KKLGEGAFGEVYKGTLKGKGGVEVEVAVKTL--KEDASEQQIEEFLREARLMRKL----D | 58 |
| Query: | 251 | CPFIVCMSYAFHTPDKLSFILDLMNGGDLHYHLSQHG--VFSEADMRFYAAEIILGLEHM | 308 |
| | | \| \|\| + + \| +++ \| \|\|\|\| +\| ++ \| +\|+ +\| +\| \|+\|++ | |
| Sbjct: | 59 | HPNIVKLLGVCTEEEPLMIVMEYMEGGDLLDYLRKNRPKELSLSDLLSFALQIARGMEYL | 118 |
| Query: | 309 | HNRFVVYRDLKPANILLDEHGHVRISDLGLACDFSKKKPHASVGTHG----YMAPEVLQK | 364 |
| | | ++ \|+\|\|\| \| \|+ \|+ \|+\|+\| \|\|\| \| + + +\|\|\|\| \|+ | |
| Sbjct: | 119 | ESKNFVHRDLAARNCLVGENKTVKIADFGLARDLYDDDYYRKKKSPRLPIRWMAPESLKD | 178 |
| Query: | 365 | GVAYDSSADWFSLGCMLFKLL-RGHSPFRQHKTKDKHEIDRMTLTMAVELPDSFSPELRS | 423 |
| | | \| + \| +\| +\| \| +\|+++ \| \|\|+ ++ ++ + + \| + \|+ | |
| Sbjct: | 179 | GK-FTSESDVWSFGVLLWEIFTLGESPY--PGMSNEEVLEYLKKGYRLPQPPNCPDEIYD | 235 |
| Query: | 424 | LLEGLLQRDVNRR 436 | |
| | | \|+ \| \| | |
| Sbjct: | 236 | LMLQCWAEDPEDR 248 | |

TABLE 9I
Domain Analysis of NOV9

gnl|Smart|smart00315, RGS, Regulator of G protein signalling domain;
RGS family members are GTPase-activating proteins for heterotrimeric
G-protein alpha-subunits. (SEQ ID NO:107)
CD-Length = 119 residues, 100.0% aligned
Score = 100 bits (248), Expect = 3e−22

| | | | |
|---|---|---|---|
| Query: | 54 | TFEKIFSQKLGYLLFRDFCLNHLEEARPLVEFYEEIKKYEKLETEEERVARSREIFDSYI | 113 |
| | | + \| + +\| \|\|\|\|+\| + \| +\|\|+ +++++\| \| \|\|\|\| ++++\|\|+\| \|+ | |
| Sbjct: | 1 | SLESLLRDPIGRLLFREFLESEFSE--ENLEFWLAVEEFKKAEDEEERRSKAKEIYDRYL | 58 |
| Query: | 114 | MKELLACSHPFSKSATEHVQGHLGKKQVPPDLFQPYIEEICQNLRGDVFQKFIESDKFTR | 173 |
| | | \| ++ +\| ++ \|\|\|\|\| \|\|+ + \| \| + +\|+\|\|\| + \| | |
| Sbjct: | 59 | SPNAPKE-VNLDSDLREEIEENLKNEEPPPDLFDEAQEEVYELLEKDSYPRFLESDYYLR | 117 |
| Query: | 174 | FC 175 | |
| | | \| | |
| Sbjct: | 118 | FL 119 | |

TABLE 9J
Domain Analysis of NOV9

gnl|Smart|smart00233, PH, Pleckstrin homology domain.; Domain commonly
found in eukaryotic signalling proteins. The domain family possesses
multiple functions including the abilities to bind inositol
phosphates, and various proteins. PH domains have been found to
possess inserted domains (such as in PLC gamma, syntrophins) and to be
inserted within other domains. Mutations in Brutons tyrosine kinase
(Btk) within its PH domain cause X-linked agammaglobulinaemia (XLA) in
patients. Point mutations cluster into the positively charged end of
the molecule around the predicted binding site for
phosphatidylinositol lipids. (SEQ ID NO:108)
CD-Length = 104 residues, 95.2% aligned
Score = 62.0 bits (149), Expect = 1e−10

| | | | |
|---|---|---|---|
| Query: | 539 | IMHGYMSKMGNPFLTQWQRRYFYLFPNRLEW-----RGEGEAPQSLLRMEEIQ---SVEE | 590 |
| | | \| \|++ \| + \|++\|\|\| \|\| \| + + \|+ + + + + | |
| Sbjct: | 2 | IKEGWLLKKSSGGKKSWKKRYFVLFNGVLLYYKSKKKKSSSKPKGSIPLSGCTVREAPDS | 61 |
| Query: | 591 | TQIKERKCLLLKIRGGKQFILQCDSDPELVQWKKELRDA 629 | |
| | | \|++ \| + \| +\|\| +\|+ \| +\| + \|\| \| | |
| Sbjct: | 62 | DSDKKKNCFEIVTPDRKTLLLQAESEEERKEWVEALRKA 100 | |

TABLE 9K
Domain Analysis of NOV9

gnl|Pfam|pfam00169, PH, PH domain. PH stands for pleckstrin homology.
(SEQ ID NO:109)
CD-Length = 100 residues, 97.0% aligned
Score = 55.5 bits (132), Expect = 1e−08

| | | | |
|---|---|---|---|
| Query: | 539 | IMHGYMSKMGNPFLTQWQRRYFYLFPNRLEW---RGEGEAPQSLLTMEEIQSVEETQIKE | 595 |
| | | \|++ \| +\|++\|\|\|+\|\| + \| + + + \|+ + + + + | |
| Sbjct: | 2 | VKEGWLLKKSTVKKKRWKKRYFFLFNDVLIYYKDKKKSYEPKGSIPLSGCSVEDVPDSEF | 61 |
| Query: | 596 | RKCLLLKIR---GGKQFILQCDSDPELVQWKKELRDA 629 | |
| | | ++ ++\| \| + \|\|\|\| +\|+ \| \| \| ++ \| | |
| Sbjct: | 62 | KRPNCFQLRSRDGKETFILQAESEEERQDWIKAIQSA 98 | |

TABLE 9L
Domain Analysis of NOV9

gnl|Smart|smart00133, S_TK_X, Extension to Ser Thr-type protein
kinases (SEQ ID NO:110)
CD-Length = 63 residues, 87.3% aligned
Score = 42.7 bits (99), Expect = 7e−05

| | | | |
|---|---|---|---|
| Query: | 454 | RSLDWQMVFLQKYPPPLIPPRGEVNAADAFDIGSFDEEDTKGIKQEVAETVFDTINAETD | 513 |
| | | \| +\|\| + ++ \|\| +\| \| +\|\| \| \| ++ \| \| +\|+\| | |
| Sbjct: | 1 | RGIDWDKLENKEIEPPFVPKVK-----SPTDTSNFDPEFT---EESPVLTPVDPPLSESD | 52 |
| Query: | 514 | RLE 516 | |
| | | + \| | |
| Sbjct: | 53 | QDE 55 | |

Eukaryotic protein kinases are enzymes that belong to a very extensive family of proteins which share a conserved catalytic core common with both serine/threonine and tyrosine protein kinases. There are a number of conserved regions in the catalytic domain of protein kinases. In the N-terminal extremity of the catalytic domain there is a glycine-rich stretch of residues in the vicinity of a lysine residue, which has been shown to be involved in ATP binding. In the central part of the catalytic domain there is a conserved aspartic acid residue which is important for the catalytic activity of the enzyme.

The beta-adrenergic receptor kinase (beta ARK) catalyses the phosphorylation of the activated forms of the beta 2-adrenergic receptor (beta 2AR). The interaction between receptor and kinase is independent of second messengers and appears to involve a multipoint attachment of kinase and substrate with the specificity being restricted by both the primary amino acid sequence and conformation of the substrate. Kinetic, functional and sequence information reveals that rhodopsin kinase and beta ARK are closely related, suggesting they are members of a family of G-protein-coupled receptor kinases.

The beta-adrenergic signaling cascade is an important regulator of myocardial function. Significant alterations of this pathway are associated with several cardiovascular diseases, including congestive heart failure (CHF). CHF patients share several similar features, such as reduced cardiac contractility and neurohumoral activation to compensate the impaired cardiac function. In CHF patients, the cardiac renin-angitensin (RA) system, receptors, GTP-binding proteins, and their effector molecules are inevitably exposed to chronically elevated neurohumoral stimulation. A widely recognized concept is that a chronic increase in such stimulation can desensitize target cell receptors and the post-receptor signal transducing pathway. Included in these alterations is increased activity and expression of G protein-coupled receptor kinases (GRKs), such as the beta-adrenergic receptor kinase (beta ARK1), which phosphorylate and desensitize beta-adrenergic receptors (beta ARs). A body of evidence is accumulating that suggests that GRKs, in particular beta ARK1, are critical determinants of cardiac function under normal conditions and in disease states. Transgenic mice with myocardial-targeted alterations of GRK activity have shown profound changes in the in vivo functional performance of the heart. Included in these studies is the compelling finding that inhibition of beta ARK1 activity or expression significantly enhances cardiac function and potentiates beta AR signaling in failing cardiomyocytes. An uncoupling of beta2-adrenoceptors has been attributed to an increased activity and gene expression of beta-adrenergic receptor kinase in failing myocardium, leading to phosphorylation and uncoupling of receptors. The important physiological function of GRK2 as a modulator of the efficacy of GPCR signal transduction systems is exemplified by its relevance in cardiovascular physiopathology as well as by its emerging role in the regulation of chemokine receptors.

The disclosed NOV9 nucleic acid of the invention encoding a Beta-adrenergic receptor kinase-like protein includes the nucleic acid whose sequence is provided in Table 9A or a fragment thereof. The invention also includes a mutant or variant nucleic acid any of whose bases may be changed from the corresponding base shown in Table 9A while still encoding a protein that maintains its Beta-adrenergic receptor kinase-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 2 percent of the bases may be so changed.

The disclosed NOV9 protein of the invention includes the Beta-adrenergic receptor kinase-like protein whose sequence is provided in Table 9B. The invention also includes a mutant or variant protein any of whose residues may be changed from the corresponding residue shown in Table 9B while still encoding a protein that maintains its Beta-adrenergic receptor kinase-like activities and physiological functions, or a functional fragment thereof. In the mutant or variant protein, up to about 1 percent of the residues may be so changed.

The protein similarity information, expression pattern, and map location for the beta- adrenergic receptor kinase-like protein and the NOV9 proteins disclosed herein suggest that this beta-adrenergic receptor kinase may have important structural and/or physiological functions characteristic of the Ser/Thr protein kinases family. Therefore, the nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo (vi) biological defense weapon.

The NOV9 nucleic acids and proteins of the invention are useful in potential diagnostic and therapeutic applications implicated in various diseases and disorders described below and/or other pathologies. For example, the compositions of the present invention will have efficacy for treatment of patients suffering from heart failure, hypertension, secondary pathologies caused by heart failure and hypertension, and other diseases, disorders and conditions of the like. Additionally, the compositions of the present invention may have efficacy for treatment of patients suffering from conditions associated with the role of GRK2 in brain and in the regulation of chemokine receptors.. The NOV9 nucleic acid, or fragments thereof, may further be useful in diagnostic applications, wherein the presence or amount of the nucleic acid or the protein are to be assessed.

NOV9 nucleic acids and polypeptides are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. For example the disclosed NOV9 protein have multiple hydrophilic regions, each of which can be used as an immunogen. In one embodiment, contemplated NOV9 epitope is from about amino acids 40 to 70. In another embodiment, the comtemplated NOV9 epitope is from about amino acids 80 to 110. In further embodiments, the contemplated NOV9 epitope is from about amino acids 120 to 200, from about amino acids 220 to 245, from about amino acids 250 to 280, or from about amino acids 290 to 340. This novel protein also has value in development of powerful assay system for functional analysis of various human disorders, which will help in understanding of pathology of the disease and development of new drug targets for various disorders.

NOV10

A disclosed NOV10 nucleic acid of 3003 nucleotides (also referred to as AC058790_da25) encoding an alpha-mannosidase-like protein is shown in Table 10A. An open reading frame was identified beginning with an ATG initiation codon at nucleotides 57-59 and ending with a TAA codon at nucleotides 2946-2948. A putative untranslated region upstream from the initiation codon and downstream from the termination codon is underlined in Table 10A. The start and stop codons are in bold letters. Single nucleotide polymorphism data is included in Example 3.

TABLE 10A
NOV10 nucleotide sequence.

(SEQ ID NO:25)

GGTATCATACTCCAGCAAGCGCACATCATCAGTGACGTCGATCACGATGCATCGTCATGGCGGCAGCGCCGTTCTTGAAG
CACTGGCGCACCACTTTTGAGCGGGTGGAGAAGTTCGTGTCCCCGATCTACTTCACCGACTGTAACCTCCGCGGCAGGCT
TTTTGGGGCCAGCTGCCCTGTGGCTGTGCTCTCCAGCTTCCTGACGCCGGAGAGACTTCCCTACCAGGAGGCAGTCCAGC
GGGACTTCCGCCCCGCGCAGGTCGGCGACAGCTTCGGACCCACATGGTGGACCTGCTGGTTCCGGGTGGAGCTGACCATC
CCAGAGGCATGGGTGGGCCAGGAAGTTCACCTTTGCTGGGAAAGTGATGGAGAAGGTCTGGTGTGGCGTGATGGAGAACC
TGTCCAGGGTTTAACCAAAGAGGGTGAGAAGACCAGCTATGTCCTGACTGACAGGCTGGGGGAAAGAGACCCCCGAAGCC
TCACTCTCTATGTGGAAGTAGCCTGCAATGGGCTCCTGGGGGCCGGGAAGGGAAGCATGATTGCAGCCCCTGACCCTGAG
AAGATGTTCCAGCTGAGCCGGGCTGAGCTAGCTGTGTTCCACCGGGATGTCCACATGCTCCTGGTGGATCTGGAGCTGCT
GCTGGGCATAGCCAAGGCGCAGCAGCTGGAATGGGTGAAGAGCCGCTACCCTGGCCTGTACTCCCGCATCCAGGAGTTTG
CGTGCCGTGGGCAGTTTGTGCCTGTGGGGGGCACCTGGGTGGAGATGGATGGGAACCTGCCCAGTGGAGAGGCCATGGTG
AGGCAGTTTTTGCAGGGCCAGAACTTCTTTCTGCAGGAGTTTGGGAAGATGTGCTCTGAGTTCTGGCTGCCGGACACCTT
TGGCTACTCAGCACAGCTCCCCCAGATCATGCACGGCTGTGGCATCAGGCGCTTTCTCACCCAGAAATTGAGCTGGAATT
TGGTGAACTCCTTCCCACACCATACATTTTTCTGGGAGGGCCTGGATGGCTCCCGTGTACTGGTCCACTTCCCACCTGGC
GACTCCTATGGGATGCAGGGCAGCGTGGAGGAGGTGCTGAAGACCGTGGCCAACAACCGGGACAAGGGGCGGGCCAACCA
CAGTGCCTTCCTCTTTGGCTTTGGGGATGGGGGTGGTGGCCCCACCCAGACCATGCTGGACCGCCTGAAGCGCCTGAGCA
ATACGGATGGGCTGCCCAGGGTGCAGCTATCTTCTCCAAGACAGCTCTTCTCAGCACTGGAGAGTGACTCAGAGCAGCTG
TGCACGTGGGTTGGGGAGCTCTTCTTGGAGCTGCACAATGGCACATACACCACCCATGCCCAGATCAAGAAGGGGAACCG
GGAATGTGAGCGGATCCTGCACGACGTGGAGCTGCTCAGTAGCCTGGCCCTGGCCCGCAGTGCCCAGTTCCTATACCCAG
CAGCCCAGCTGCAGCACCTCTGGAGGCTCCTTCTTCTGAACCAGTTCCATGATGTGGTGACTGGAAGCTGCATCCAGATG
GTGGCAGAGGAAGCCATGTGCCATTATGAAGACATCCGTTCCCATGGCAATACACTGCTCAGCGCTGCAGCCGCAGCCCT
GTGTGCTGGGGAGCCAGGTCCTGAGGGCCTCCTCATCGTCAACACACTGCCCTGGAAGCGGATCGAAGTGATGGCCCTGC
CCAAACCGGGCGGGGCCCACAGCCTAGCCCTGGTGACAGTGCCCAGCATGGGCTATGCTCCTGTTCCTCCCCCCACCTCA
CTGCAGCCCCTGCTGCCCCAGCAGCCTGTGTTCGTAGTGCAAGAGACTGATGGCTCCGTGACTCTGGACAATGGCATCAT
CCGAGTGAAGCTGGACCCAACTGGTCGCCTGACGTCCTTGGTCCTGGTGGCCTCTGGCAGGGAGGCCATTGCTGAGGGCG
CCGTGGGGAACCAGTTTGTGCTATTTGATGATGTCCCCTTGTACTGGGATGCATGGGACGTCATGGACTACCACCTGGAG
ACACGGAAGCCTGTGCTGGGCCAGGCAGGGACCCTGGCAGTGGGCACCGAGGGCGGCCTGCGGGGCAGCGCCTGGTTCTT
GCTACAGATCAGCCCCAACAGTCGGCTTAGCCAGGAGGTTGTGCTGGACGTTGGCTGCCCCTATGTCCGCTTCCACACCG
AGGTACACTGGCATGAGGCCCACAAGTTCCTGAAGGTGGAGTTCCCTGCTCGCGTGCGGAGTTCCCAGGCCACCTATGAG
ATCCAGTTTGGGCACCTGCAGCGACCTACCCACTACAATACCTCTTGGGACTGGGCTCGATTTGAGGTGTGGGCCCATCG
CTGGATGGATCTGTCAGAACACGGCTTTGGGCTGGCCCTGCTCAACGACTGCAAGTATGGCGCGTCAGTGCGAGGCAGCA
TCCTCAGCCTCTCGCTCTTGCGGGCGCCTAAAGCCCCGGACGCTACTGCTGACACGGGGCGCCACGAGTTCACCTATGCA
CTGATCTTCAGCAAGGGCTCTTTCCAGGATGCTGGCGTTATCCAAGCTGCCTACAGCCTAAACTTCCCCCTGTTGGCTCT
GCCAGCCCCCAGCCCAGCGCCCGCCACCTCCTGGAGTGCGTTTTCCGTGTCTTCACCCGCGGTCGTATTGGAGACCGTCA
AGCAGGCGGAGAGCAGCCCCCAGCGCCGCTCGCTGGTCCTGAGGCTGTATGAGGCCCACGGCAGCCACGTGGACTGCTGG
CTGCACTTGTCGCTGCCGGTTCAGGAGGCCATCCTCTGCGATCTCTTGGAGCGACCAGACCCTGCTGGCCACTTGACTTC
GGGACAACCGCCTGAAGCTCACCTTTTCTCCCTTCCAAGTGCTGTCCCTGTTGCTCGTGCTTCAGCCTCCGCCACACTGA
GTCCCTGGGGCTGGGGTTTTGTTTGTAGAAGGCTCTGGGGACTCCTAATTTCTGCTTCCCCAGCCTAAAGCAGGGATCAG
TCTTTTCTTGTGGAATAAATCCTTGGATCGGGAAAAAAAAAAA

In a search of public sequence databases, the NOV10 nucleic acid sequence, located on chromsome 15 has 2371 of 2390 bases (99%) identical to a alpha-mannosidase mRNA from Homo sapiens, (GENBANK-ID: AF044414|acc: AF044414.2) (E=0.0). Public nucleotide databases include all GenBank databases and the GeneSeq patent database.

The disclosed NOV10 polypeptide (SEQ ID NO: 26) encoded by SEQ ID NO: 25 has 963 amino acid residues and is presented in Table 10B using the one-letter amino acid code. Signal P. Psort and/or Hydropathy results predict that NOV10 does not have a signal peptide and is likely to be localized in the peroxisome (microbody) with a certainty of 0.7480. In other embodiments, NOV10 is also likely to be localized to the mitochondrial membrane space with a certainty of 0.4539, to the mitochondrial intermembrane space with a certainty of 0.4027, or to the lysosome (lumen) with a certainty of 0.2317.

TABLE 10B
Encoded NOV10 protein sequence.

(SEQ ID NO:26)

MAAAPFLKHWRTTFERVEKFVSPIYFTDCNLRGRLFGASCPVAVLSSFLTPERLPYQEAVQRDFRPAQVGDS
FGPTWWTCWFRVELTIPEAWVGQEVHLCWESDGEGLVWRDGEPVQGLTKEGEKTSYVLTDRLGERDPRSLTL
YVEVACNGLLGAGKGSMIAAPDPEKMFQLSRAELAVFHRDVHMLLVDLELLLGIAKAQQLEWVKSRYPGLYS
RIQEFACRGQFVPVGGTWVEMDGNLPSGEAMVRQFLQGQNFFLQEFGKMCSEFWLPDTFGYSAQLPQIMHGC
GIRRFLTQKLSWNLVNSFPHHTFFWEGLDGSRVLVHFPPGDSYGMQGSVEEVLKTVANNRDKGRANHSAFLF
GFGDGGGGPTQTMLDRLKRLSNTDGLPRVQLSSPRQLFSALESDSEQLCTWVGELFLELHNGTYTTHAQIKK
GNRECERILHDVELLSSLALARSAQFLYPAAQLQHLWRLLLLNQFHDVVTGSCIQMVAEEAMCHYEDIRSHG
NTLLSAAAAALCAGEPGPEGLLIVNTLPWKRIEVMALPKPGGAHSLALVTVPSMGYAPVPPPTSLQPLLPQQ
PVFVVQETDGSVTLDNGIIRVKLDPTGRLTSLVLVASGREAIAEGAVGNQFVLFDDVPLYWDAWDVMDYHLE
TRKPVLGQAGTLAVGTEGGLRGSAWFLLWISPNSRLSQEVVLDVGCPYVRFHTEVHWHEAHKFLKVEFPARV
RSSQATYEIQFGHLQRPTHYNTSWDWARFEVWAHRWMDLSEHGFGLALLNDCKYGASVRGSILSLSLLRAPK
APDATADTGRHEFTYALMPHKGSFQDAGVIQAAYSLNFPLLALPAPSPAPATSWSAFSVSSPAVVLETVKQA
ESSPQRRSLVLRLYEAHGSHVDCWLHLSLPVQEAILCDLLERPDPAGHLTSGQPPEAHLFSLPSAVPVARAS
ASATLSPWGWGFVCRRLWGLLISASPA

A search of sequence databases reveals that the NOV10 amino acid sequence has 764 of 771 amino acid residues (99%) identical to, and 767 of 771 amino acid residues (99%) similar to, the 1062 amino acid residue alpha-mannosidase protein from Homo sapiens (Q9UL64) (E=0.0). Public amino acid databases include the GenBank databases, SwissProt, PDB and PIR.

NOV10 was derived from a pool of the following tissues: Adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea, uterus, Bone, Cervix, Chorionic Villus, Colon, Liver, Lung, Lymph node, Lymphoid tissue, Ovary, Peripheral Blood, Skin, Stomach, Tonsils, Whole Organism. Thus, it is expressed in at least some of the above tissues. This information was derived by determining the tissue sources of the sequences that were included in the invention including but not limited to SeqCalling sources, Public EST sources, Genomic Clone sources, Literature sources, and/or RACE sources.

The disclosed NOV10 polypeptide has homology to the amino acid sequences shown in the BLASTP data listed in Table 10C.

TABLE 10C
BLAST results for NOV10

| | | Length | Identity | Positives | |
|---|---|---|---|---|---|
| Gene Index/Identifier | Protein/Organism | (aa) | (%) | (%) | Expect |
| ptnr: SPTREMBL- | ALPHA MANNOSIDASE | 1062 | 763/771 | 767/771 | 0.0 |
| ACC: Q9UL64 | 6A8B - Homo | | (99%) | (99%) | |
| | sapiens | | | | |
| ptnr: SPTREMBL- | HYPOTHETICAL 115.8 | 1040 | 715/722 | 718/722 | 0.0 |
| ACC: Q9NTJ4 | KDA PROTEIN - | | (99%) | (99%) | |
| | Homo sapiens | | | | |
| ptnr: TREMBLNEW- | SIMILAR TO | 1039 | 635/730 | 692/730 | 0.0 |
| ACC: AAH16253 | MANNOSIDASE, | | (89%), | (94%) | |
| | ALPHA, CLASS 2C, | | | | |
| | MEMBER 1 | | | | |
| ptnr: SWISSPROT- | Alpha-mannosidase | 1040 | 625/731 | 661/731 | 0.0 |
| ACC: P21139 | (EC 3.2.1.24) | | (85%) | (90%) | |
| ptnr: SPTREMBL- | ALPHA-MANNOSIDASE - | 425 | 425/425 | 425/425 | 0.0 |
| ACC: Q13358 | Homo sapiens | | (100%) | (100%) | |

The homology between these and other sequences is shown graphically in the ClustalW analysis shown in Table 10D. In the ClustalW alignment of the NOV10 protein, as well as all other ClustalW analyses herein, the black outlined amino acid residues indicate regions of conserved sequence (i.e., regions that may be required to preserve structural or functional properties), whereas non-highlighted amino acid residues are less conserved and can potentially be altered to a much broader extent without altering protein structure or function.

[image]

[image]

Table 10E lists the domain description from DOMAIN analysis results against NOV10. This indicates that the NOV10 sequence has properties similar to those of other proteins known to contain this domain.

TABLE 10E
Domain Analysis of NOV10

| Model | Description | Score | E−value |
|---|---|---|---|
| Glyco_hydro_38 (InterPro) | Glycosyl hydrolases family 38 | 140.5 | 1e−39 |
| (SEQ ID NO:111) | | | |

Glyco_hydro_38: domain 1 of 2, from 230 to 332:
score 89.2, E = 5.4e−25

| | | | |
|---|---|---|---|
| | | *->vtGGWVMnDEAttHyedlIdQlteGHqfLeenfGsdvkPkvgWsIDP | |
| | | \|+\|+\|\|+ \| + +++\|++++\|++ \|+ \|+ ++\|\| + +\|++\|+ | |
| AC058790_d | 230 | VGGTWVEMDGNLPSGEAMVRQFLQGQNFFLQEFT--KMCSEFWLPDT | 274 |
| | | FGHSatmPyLlraqaGfdgflIgRihYadKksfaetkqleFvWRqswslt | |
| | | \|\|+\|\|++\|++ + \|+ +\|\|+\|++++++ +\|\|++++ \|+\| \|+ | |
| AC058790_d | 275 | FGYSAQLPQIM-HGCGIRRFLTQKLSWNLVNSFPHHT---FFWE---GLD | 317 |
| | | gstdlfthmmpfysYd<-* | |
| | | \|\| +++\| +\| +\|\|+ | |
| AC058790_d | 318 | GS-RVLVHFPPGDSYG 332 | |

Glyco_hydro_38: domain 2 of 2, from 410 to 490;
score 49.2, E = 1.7e−13

| | | | |
|---|---|---|---|
| | | *->pYAdepdeGKPeYWTGYFTSRPalKrldRqlehlLrsaEilatqlsv | |
| | | ++ +\|++ \| + \|++\|++++ \|+ +\|++\| \|+++\|+\|++++ + | |
| AC058790_d | 410 | TWVGELFL---ELHNGTYTTHAQIKKGNRECERILHDVELLSSLALA | 453 |
| | | laggskiegsyAiKleklyeqleelRralaLfQHHDAiTGTakghVv<-* | |
| | | +++++ + \|\|+ \|+\| \|+\|+\|+\|\|++\|\|+++\|+\|+ | |
| AC058790_d | 454 | RS-AQFLYPA-----A----QLQHLWRLLLLNQFHDVVTGSCIQMVA | 490 |

Glycosyl hydrolases are key enzymes of carbohydrate metabolism. Lysosomal alpha-mannosidase is necessary for the catabolism of N-linked carbohydrates released during glycoprotein turnover. The enzyme catalyzes the hydrolysis of terminal, non-reducing alpha-D-mannose residues in alpha-D-mannosides, and can cleave all known types of alpha-mannosidic linkages. While alpha-mannosidases were classified as enzymes that process newly formed N-glycans or degrade mature glycoproteins, two endoplasmic reticulum (ER) alpha-mannosidases with previously assigned processing roles, have important catabolic activities. The ER/cytosolic mannosidase may be involved in the degradation of dolichol intermediates that are not needed for protein glycosylation, whereas the soluble form of Man9-mannosidase is responsible for the degradation of glycans on defective or malfolded proteins that are specifically retained and broken down in the ER. The degradation of oligosaccharides derived from dolichol intermediates by ER/cytosolic mannosidase explains why cats and cattle with alpha-mannosidosis store and excrete some unexpected oligosaccharides containing only one GlcNAc residue. Similarly, the action of ER/cytosolic mannosidase, followed by the action of the recently described human lysosomal alpha(1→6)-mannosidase, together explain why alpha-mannosidosis patients store and excrete large amounts of oligosaccharides that resemble biosynthetic intermediates, rather than partially degraded glycans. The relative contributions of the lysosomal and extra-lysosomal catabolic pathways can be derived by comparing the ratio of trisaccharide Man beta (1→4)GlcNAc beta (1→4)GlcNAc to disaccharide Man beta (1→4)GlcNAc accumulated in tissues from goats with beta-mannosidosis. A similar determination in human beta-mannosidosis patients is not possible because the same intermediate, Man beta (1→4)-GlcNAc is a product of both pathways. Based on inhibitor studies with pyranose and furanose analogues, alpha-mannosidases may be divided into two groups. Those in Class 1 are (1→2)-specific enzymes like Golgi mannosidase I, whereas those in Class 2, like lysosomal alpha-mannosidase, can hydrolyse (1→2), (1→3) and (1→6) linkages. A similar classification has been derived from protein sequence homologies. It is possible to speculate about their probable evolution from two primordial genes. The first would have been a Class 1 ER enzyme involved in the degradation of glycans on incompletely assembled or malfolded glycoproteins. The second would have been a Class 2 lysosomal enzyme responsible for turnover. Later, other alpha-mannosidases, with new processing or catabolic functions, would have developed from these, by loss or gain of critical insertion or retention sequences, to yield the full complement of alpha-mannosidases known today (Glycobiology October 1994 ;4(5):551-66). Defects in the lysosomal alpha-mannosidase gene cause lysosomal alpha-mannosidosis (AM), a lysosomal storage disease characterized by the accumulation of unbranched oligo-saccharide chains. Depending on the clinical findings at the age of onset, a severe infantile (type I) and a mild juvenile (type II) form of alpha-mannosidosis are recognized. Furthermore, variability in clinical expression of the disease is seen within each type. Some of the disease features are: susceptibility to infection, vomiting, coarse features, macroglossia, flat nose, large clumsy ears, widely spaced teeth, large head, big hands and feet, tall stature, slight hepatosplenomegaly, muscular hypotonia, lumbar gibbus, radiographic skeletal abnormalities, dilated cerebral ventricles, lenticular opacities, hypogammaglobulinemia, ‘storage cells’ in the bone marrow, and vacuolated lymphocytes in the bone marrow and blood.

The disclosed NOV10 nucleic acid of the invention encoding a Alpha-mannosidase-like protein includes the nucleic acid whose sequence is provided in Table 10A or a fragment thereof. The invention also includes a mutant or variant nucleic acid any of whose bases may be changed from the corresponding base shown in Table 10A while still encoding a protein that maintains its Alpha-mannosidase-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 2 percent of the bases may be so changed.

The disclosed NOV10 protein of the invention includes the Alpha-mannosidase-like protein whose sequence is provided in Table 10B. The invention also includes a mutant or variant protein any of whose residues may be changed from the corresponding residue shown in Table 10B while still encoding a protein that maintains its Alpha-mannosidase-like activities and physiological functions, or a functional fragment thereof. In the mutant or variant protein, up to about 1 percent of the residues may be so changed.

The protein similarity information, expression pattern, and map location for the alpha-mannosidase-like protein and the NOV10 protein disclosed herein suggest that this alpha-mannosidase-like protein may have important structural and/or physiological functions characteristic of the mannosidase protein family. Therefore, the nucleic acids and proteins of the invention are useful in potential diagnostic and therapeutic applications and as a research tool. These applications include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo (vi) biological defense weapon.

The NOV10 nucleic acids and proteins of the invention are useful in potential diagnostic and therapeutic applications implicated in various diseases and disorders described below and/or other pathologies. For example, the compositions of the present invention will have efficacy for treatment of patients suffering from alpha-mannosidosis, beta-mannosidosis, other storage disorders, peroxisomal disorders such as zellweger syndrome, infantile refsum disease, rhizomelic chondrodysplasia (chondrodysplasia punctata, rhizomelic), and hyperpipecolic acidemia and other diseases, disorders and conditions of the like. Since mannosidoses are found not only in humans, but also in animals, the nucleic acids and proteins of the this invention may be useful in treating animals with mannosidoses or other storage diseases, and other diseases, disorders and conditions of the like. Additionally, the compositions of the present invention may have efficacy for treatment of patients suffering from conditions associated with the role of GRK2 in brain and in the regulation of chemokine receptors.. The NOV10 nucleic acid, or fragments thereof, may further be useful in diagnostic applications, wherein the presence or amount of the nucleic acid or the protein are to be assessed.

NOV10 nucleic acids and polypeptides are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. For example the disclosed NOV10 protein have multiple hydrophilic regions, each of which can be used as an immunogen. In one embodiment, contemplated NOV10 epitope is from about amino acids 5 to 20. In another embodiment, the comtemplated NOV10 epitope is from about amino acids 40 to 80. In further embodiments, the contemplated NOV10 epitope is from about amino acids 110 to 180, from about amino acids 200 to 230, from about amino acids 300 to 370, from about amino acids 375 to 450, from about amino acids 650 to 680, from about amino acids 690 to 770, from about amino acids 790 to 820, from about amino acids 850 to 880, or from about amino acids 900 to 920. This novel protein also has value in development of powerful assay system for functional analysis of various human disorders, which will help in understanding of pathology of the disease and development of new drug targets for various disorders.

NOV11

NOV11 includes three novel C1q-related factor-like proteins disclosed below. The disclosed sequences have been named NOV11a, NOV11b, and NOV11c. Single nucleotide polymorphism data is discussed below in Example 4.

NOV11a

A disclosed NOV11a nucleic acid of 805 nucleotides (also referred to as GM57107065_da1) encoding an C1q-related factor-like protein is shown in Table 11A. An open reading frame was identified beginning with an ATG initiation codon at nucleotides 83-85 and ending with a TGA codon at nucleotides 797-799. Putative untranslated regions are upstream from the initiation codon and downstream from the termination codon.

TABLE 11A
NOV11a nucleotide sequence.

(SEQ ID NO:27)

GAGTGAGGAAGATTTGCTGGCCCTGGCAGCGTCGCGGCTGAGCCGCCGCAAGAGGGTGGCGGGCGCGGCCGTCGGAGTGG
CCATGGTGCTGCTGCTGCTGGTGGCCATCCCGCTGCTGGTGCACAGCTCCCGCGGGCCAGCGCACTACGAGATGCTGGGT
CGCTGCCGCATGGTGTGCGACCCGCATGGGCCCCGTGGCCCTGGTCCGGACGGCGCGCCTGCTTCCGTGCCCCCCTTCCC
GCCAGGCGCCAAGGGAGAGGTGGGCCGGTGCGGGAAAGCAGGCCTGAGGGGGCCCCCTGGACCACCAGGTCCAAGAGGGC
CCCCAGGAGAACCCGGCAGGCCAGGCCCCCCGGGCCCTCCCGGTCCAGGTCCGGGCGGGGTGGCGCCCGCTGCCGGCTAC
GTGCCTCGCATTGCTTTCTACGCGGGCCTGCGGCGGCCCCACGAGGGTTACGAGGTGCTGCGCTTCGACGACGTGGTGAC
CAACGTGGGCAACGCCTACGAGGCAGCCAGCGGCAAGTTTACTTGCCCCATGCCAGGCGTCTACTTCTTCGCTTACCACG
TGCTCATGCGCGGCGGCGACGGCACCAGCATGTGGGCCGACCTCATGAAGAACGGACAGGTCCGGGCCAGCGCCATTGCT
CAGGACGGGGACCAGAACTACGACTACGCCAGCAACAGCGTCATTCTGCACCTGGACGTGGGCGACGAGGTCTTCATCAA
GCTGGACCCCGGGAAAGTGCACGGCGGCAACACCAACAAGTACAGCACCTTCTCCGGCTTCATCATCTACCCCGACTGAG
CCGGC

In a search of public sequence databases, the NOV11a nucleic acid, located on chromsome 12, has 565 of 787 bases (71%) identical to a C1q-related factor mRNA from Homo sapiens, (GENBANK-ID: AF095154) (E=9.9e⁻⁶⁸). Public nucleotide databases include all GenBank databases and the GeneSeq patent database.

The disclosed NOV11a polypeptide (SEQ ID NO: 28) encoded by SEQ ID NO: 27 has 238 amino acid residues and is presented in Table 11B using the one-letter amino acid code. Signal P, Psort and/or Hydropathy results predict that NOV11a has a signal peptide and is likely to be localized extracellularly with a certainty of 0.5374. In other embodiments, NOV11a is also likely to be localized to the microbody (peroxisome) with a certainty of 0.1111, to the endoplasmic reticulum (membrane) with a certainty of 0.1000, and to the endoplasmic reticulum (lumen) with a certainty of 0.1000. The most likely cleavage site for NOV11a is between positions 15 and 16: VHS-SR.

TABLE 11b
Encoded NOV11a protein sequence.

(SEQ ID NO:28)

MVLLLLVAIPLLVHSSRGPAHYEMLGRCRMVCDPHGPRGPGPDGAPASVP
PFPPGAKGEVGRCGKAGLRGPPGPPGPRGPPGEPGRPGPPGPPGPGPGGV
APAAGYVPRIAFYAGLRRPHEGYEVLRFDDVVTNVGNAYEAASGKFTCPM
PCVYFFAYHVLMRGGDGTSMWADLMKNGQVRASAIAQDADQNYDYASNSV
ILHLDVGDEVFIFLDGGKVGNINKYSTFSGGFIIYPD

A search of sequence databases reveals that the NOV11a amino acid sequence has 184 of 258 amino acid residues (71%) identical to, and 198 of 258 amino acid residues (76%) similar to, the 258 amino acid residue C1q-related factor precursor protein from Homo sapiens (075973) (E=9.1 e⁻⁹¹). Public amino acid databases include the GenBank databases, SwissProt, PDB and PIR.

NOV11a is specifically expressed in the following tissues: brain, heart, testis, kidney, thyroid, prostate, fetal kidney, fetal skletal. It shows increased expression in cancer cell lines derived from the following tissue: colon, kidney, ovary, skin, brain. It is highly upregulated in IFN-gamma treated endothelial cells. This information was derived by determining the tissue sources of the sequences that were included in the invention including but not limited to SeqCalling sources and Taqman results.

NOV11b

A disclosed NOV11b nucleic acid of 805 nucleotides (also referred to as CG54503-02) encoding a novel C1q-related factor-like protein is shown in Table 11C. An open reading frame was identified beginning with an ATG initiation codon at nucleotides 83-85 and ending with a TGA codon at nucleotides 797-799. Putative untranslated regions are underlined and are found upstream from the initiation codon and downstream from the termination codon.

TABLE 11C
+HZ,50
NOV11b nucleotide sequence.

(SEQ ID NO:29)

GAGTGAGGAAGATTTGCTGGCCCTGGCAGCGTCGCGGCTGAGCCGCCGCAAGAGGGTGGCGGGCGCGGCCGTCGGAGTGG
CCATGGTGCTGCTGCTGCTGGTGGCCATCCCGCTGCTGGTGCACAGCTCCCGCGGGCCAGCGCACTACGAGATGCTGGGT
CGCTGCCGCATGGTGTGCGACCCGCATGGGCCCCGTGGCCCTGGTCCGGACGGCGCGCCTGCTTCCGTGCCCCCCTTCCC
GCCAGGCGCCAAGGGAGAGGTGGGCCGGCGCGGGAAAGCAGGCCTGCGGGGGCCCCCTGGACCACCAGGTCCAAGAGGGC
CCCCAGGAGAACCCGGCAGGCCAGGCCCCCCGGGCCCTCCCGGTCCAGGTCCGGGCGGGGTGGCGCCCGCTGCCGGCTAC
GTGCCTCGCATTGCTTTCTACGCGGGCCTGCGGCGGCCCCACGAGGGTTACGAGGTGCTGCGCTTCGACGACGTGGTGAC
CAACGTGGGCAACGCCTACGAGGCAGCCAGCGGCAAGTTTACTTGCCCCATGCCAGGCGTCTACTTCTTCGCTTACCACG
TGCTCATGCGCGGCGGCGACGGCACCAGCATGTGGGCCGACCTCATGAAGAACGGACAGGTCCGGGCCAGCGCCATTGCT
CAGGACGCGGACCAGAACTACGACTACGCCAGCAACAGCGTCATTCTGCACCTGGACGTGGGCGACGAGGTCTTCATCAA
GCTGGACGGCGGGAAAGTGCACGGCGGCAACACCAACAAGTACAGCACCTTCTCCGGCTTCATCATCTACCCCGACTGAG
CCGGC

In a search of public sequence databases, the NOV11a nucleic acid, located on chromsome 17q21, has 565 of 787 bases (71%) identical to a C1q-related factor mRNA from Homo sapiens, (GENBANK-ID: AF095154) (E=1.9e⁻⁶⁸). Public nucleotide databases include all GenBank databases and the GeneSeq patent database.

The disclosed NOV11b polypeptide (SEQ ID NO: 30) encoded by SEQ ID NO: 29 has 238 amino acid residues and is presented in Table 11D using the one-letter amino acid code.

The SignalP, Psort and/or Hydropathy profile for NOV11b predict that this sequence has a signal peptide and is likely to be localized extracellularly with a certainty of 0.5374, as expected by a protein similar to the C1q complement component. In other embodiments, NOV11b is also likely to be localized to the microbody (peroxisome) with a certainty of 0.1199, to the endoplasmic reticulum (membrane) with a certainty of 0.1000, and to the endoplasmic reticulum (lumen) with a certainty of 0.1000. The most likely cleavage site for NOV11b is between positions 15 and 16: VHS-SR.

TABLE 11D
Encoded NOV11b protein sequence.

(SEQ ID NO:30)

MVLLLLVAIPLLVHSSRGPAHYEMLGRCRMVCDPHGPRGPGPDGAPASVP
PFPPGAKGEVGRRGKAGLRGPPGPPGPRGPPGEPGRPGPPGPPGPGPGGV
APAAGYVPRIAFYAGLRRPHEGYEVLRFDDVVTNVGNAYEAASGKFTCPM
PGVYFFAYHVLMRGGDGTSMWADLMKNGQVRASAIAQDADQNYDYASNSV
ILHLDVGDEVFIKLDGGKVHGGNTNKYSTFSGFIIYPD

A search of sequence databases reveals that the NOV11b amino acid sequence has 184 of 258 amino acid residues (71%) identical to, and 198 of 258 amino acid residues (76%) similar to, the 258 amino acid residue C1q-related factor precursor protein from Homo sapiens (075973) (E=7.1 e⁻⁹¹). Public amino acid databases include the GenBank databases, SwissProt, PDB and PIR.

NOV11b is expressed in at least some of the following tissues: adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea, uterus, right cerebellum. This information was derived by determining the tissue sources of the sequences that were included in the invention including but not limited to SeqCalling sources, Public EST sources, Literature sources, and/or RACE sources.

The disclosed NOV11a polypeptide has homology to the amino acid sequences shown in the BLASTP data listed in Table 11E.

TABLE 11E
BLAST results for NOV11a

| Gene Index/ | | Length | Identity | | |
|---|---|---|---|---|---|
| Identifier | Protein/Organism | (aa) | (%) | Positives (%) | Expect |
| Ptnr: SWISSPROT-ACC: | C1q-related | 258 | 184/258 | 198/258 | 9.1e−91 |
| 075973 | factor precursor - | | (71%) | (76%) | |
| | Homo sapiens | | | | |
| ptnr: SWISSPROT- | C1q-related | 258 | 156/216 | 166/216 | 1.8e−78 |
| ACC: O88992 | factor precursor - | | (72%) | (76%) | |
| | Mus musculus | | | | |
| ptnr: SWISSPROT- | Gliacolin | 155 | 153/209 | 165/209 | 1.3e−77 |
| ACC: Q9ESN4 | precursor - Mus | | (73%) | (78%) | |
| | musculus | | | | |
| ptnr: SWISSPROT- | Complement C1q | 251 | 90/239 | 124/239 | 1.3e−29 |
| ACC: P02746 | subcomponent | | (37%) | (51%) | |
| ptnr: TREMBLNEW- | COMPLEMENT | 253 | 90/239 | 124/239 | 1.3e−29 |
| ACC: AAH08983 | COMPONENT 1 | | (37%) | (51%) | |

The homology between these and other sequences is shown graphically in the ClustalW analysis shown in Table 11F. In the ClustalW alignment of the NOV11 protein, as well as all other ClustalW analyses herein, the black outlined amino acid residues indicate regions of conserved sequence (i.e., regions that may be required to preserve structural or functional properties), whereas non-highlighted amino acid residues are less conserved and can potentially be altered to a much broader extent without altering protein structure or function. V,36/2

Tables 11E-11F list the domain descriptions from DOMAIN analysis results against NOV11. This indicates that the NOV11 sequence has properties similar to those of other proteins known to contain this domain.

TABLE 11E
Domain Analysis of NOV11

gnl|Smart|smart00110, C1Q, Complement component Clq domain.; Globular
domain found in many collagens and eponymously in complement Clq. When
part of full length proteins these domains form a bouquete due to the
multimerization of heterotrimers. The Clq fold is similar to that of
tumour necrosis factor. (SEQ ID NO:104)
CD-Length = 132 residues, 99.2% aligned
Score = 113 bits (283), Expect = 1e−26

| | | | |
|---|---|---|---|
| Query: | 108 | PRIAFYAGL--RRPHEGYEVLRFDDVVTNVGNAYEAASGKFTCPMPGVYFFAYHVLMRGG | 165 |
| | | \|\| \|\| \|\| + +\|\|\| \|+ \| \|+ ++\|\|\|\|\|\|+\|\|\|\|+51 +\|\|+ 30 | |
| Sbjct: | 2 | PRSAFSVIRSTNRPPPPGQPVRFDKVLYNQQGHYDPSTGKFTCPVPGVYYFSYHIESK-- | 59 |
| Query: | 166 | DGTSMWADLMKNGQVRASAIAQDADQNYDYASNSVILHLDVGDEVFIKLDGGKVHG-GNT | 224 |
| | | \| ++ \|\|\|\|\| + \| \|\| +\| \| \|\|+\|+++\|\| \| | |
| Sbjct: | 60 | -GRNVKVSLMKNGIQVMRECDEYQKGLYQVASGGALLQLRQGDQVWLELDDKKNGLYAGE | 118 |
| Query: | 225 | NKYSTFSGFIIYPD 238 | |
| | | \|\|\|\|\|\|+++\|\| | |
| Sbjct: | 119 | EVDSTFSGFLLFPD 132 | |

TABLE 11F
Domain Analysis of NOV11

gnl|Pfam|pfam00386, Clq, Clq domain. Clq is a subunit of the C1 enzyme
complex that activates the serum complement system. (SEQ ID NO:112)
CD-Length = 125 residues, 100.0% aligned
Score = 102 bits (253), Expect =3e−23

| | | | |
|---|---|---|---|
| Query: | 111 | AFYAGLR-RPHEGYEVLRFDDVVTNVGNAYEAASGKFTCPMPGVYFFAYHVLMRGGDGTS | 169 |
| | | \|\| \| \|\| + + \|\|+\|+ \| \|+ \|+\|\|\|\|\|\|+\|\|+\|+\| +\|\| + \|\|+ | |
| Sbjct: | 1 | AFTAIRSTRPPAPGQPVIFDEVLYNQQGHYDPATGKFTCPVPGLYYFNFHVSSK---GTN | 57 |
| Query: | 170 | MWADLMKNGQVRASAIAQDADQNYDYASNSVILHLDVGDEVFIKLDGGKVHG--GNTNKY | 227 |
| | | + \|\|+\|\| \| + \| \| \|\| +\| \| \|\| \|+++\|\| + +\| \| + | 227 |
| Sbjct: | 58 | VCVSLMRNGVPVMSFCDEYAKGTYQVASGGAVLQLRQGDRVWLELDDKQTNGLLGGEGVH | 117 |
| Query: | 228 | STFSGFII 235 | |
| | | \| \|\|\|\|++ | |
| Sbjct: | 118 | SVFSGFLL 125 | |

The first component of complement system is a calcium-dependent complex of the 3 subcomponents C1q, C1r, and C1s. Subcomponent C1q binds to immunoglobulin complexes with resulting serial activation of C1r (enzyme), C1s (proenzyme) and the other 8 components of complement. It contains collagen like domains. It has been shown that fibronectin binds to C1q in the same manner that it binds collagen. A major function of the fibronectins is in the adhesion of cells to extracellular materials such as solid substrata and matrices. Because fibronectin stimulates endocytosis and promotes the clearance of particulate material from the circulation, the results suggest that fibronectin functions in the clearance of C1q-coated material such as immune complexes or cellular debris. Many examples of deficiencies of C1q have been reported, most of them associated with systemic lupus erythematosus or glomerulonephritis.

The complement system plays a paradoxical role in the development and expression of autoimmunity in humans. The activation of complement in SLE contributes to tissue injury. In contrast, inherited deficiency of classic pathway components, particularly C1q, is probably associated with the development of SLE. This leads to the hypothesis that a physiologic action of the early part of the classic pathway protects against the development of SLE and implies that C1q may play a key role in this respect. C1q-deficient (C1qa−/−) mice have been shown to have increased mortality and higher titers of autoantibodies, compared with strain-matched controls. Of the C1qa−/− mice, 25% have been shown to have glomerulonephritis with immune deposits and multiple apoptotic cell bodies. Among mice without glomerulonephritis, there were significantly greater numbers of glomerular apoptotic bodies in C1q-deficient mice compared with controls. The phenotype associated with C1q deficiency was modified by background genes. These findings are compatible with the hypothesis that C1q deficiency causes autoimmunity by impairment of the clearance of apoptotic cells.

The C1q-related factor is a recently discovered protein which has homology to C1q. Since this is a relatively new discovery, very little is known about its function. But conclusions could clearly be derived from it expression pattern and it homology to C1q. Based on its expression pattern it has been suggested that this protein may be involved in motor function. The functions of C1q has been described above and include role in binding to immunoglobulin complexes, cell adhesion, autoimmunity and apoptosis, among others.

The disclosed NOV11 nucleic acid of the invention encoding a C1q-related factor-like protein includes the nucleic acid whose sequence is provided in Table 11A, 11C, or a fragment thereof. The invention also includes a mutant or variant nucleic acid any of whose bases may be changed from the corresponding base shown in Table 11A or 11C while still encoding a protein that maintains its C1q-related factor-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 29 percent of the bases may be so changed.

The disclosed NOV11 protein of the invention includes the C1q-related factor-like protein whose sequence is provided in Table 11B or 11D. The invention also includes a mutant or variant protein any of whose residues may be changed from the corresponding residue shown in Table 11B or 11D while still encoding a protein that maintains its C1q-related factor-like activities and physiological functions, or a functional fragment thereof. In the mutant or variant protein, up to about 29 percent of the residues may be so changed.

The protein similarity information, expression pattern, and map location for the C1q-related factor-like protein and nucleic acid disclosed herein suggest that this C1q-related factor may have important structural and/or physiological functions characteristic of the C1q family. Therefore, the nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo (vi) biological defense weapon.

The nucleic acids and proteins of the invention are useful in potential diagnostic and therapeutic applications implicated in various diseases and disorders described below and/or other pathologies. Based on the TaqMan data, the compositions of the present invention, will have efficacy for treatment of patients suffering from: cancer of the colon, kidney, ovary, skin and brain. Since it is over expressed in cell lines derived from these tissues it can also be used as a diagnostic marker for cancer in these tissues. The expression of the novel gene of this invention upon activation of HUVEC and the homology of the novel protein of this invention to C1q may indicate that it is secreted by endothelial cells in areas of inflammtion where Th1 cells are inflitrating the inflammation site such as Rheumatoid Arthritis and Inflammatory Bowel Disease. Based on its homology to C1q, the novel protein could be either pro-inflammatory activating the complement cascade and be a useful target for a monoclonal antibody to block this effect. Alternatively, this protein may act as a competitor of C1q and so act to down regulate complement mediated damage of endothelial cells. In this case it could be used as a protein therapeutic. IFN gamma also induces production of this protein by airway epithelilial cell lines NCI-H292 and dermal fibroblasts indicating again that it may play a role in Th1 inflammatory diseases such as rheumatoid arthritis, multiple sclerosis, inflammatory bowel diseases and psoriasis and other diseases, disorders and conditions of the like. Because of its high homology to C1q-related factor, this novel protein may also play a role in disorders of the nervous system involved in motor function.

Based on its homology to C1q, the novel protein of invention may also play a role in the pathogenesis of systemic lupus erythematosus and glomerulonephritis and therefore could be used for detection and treatment of these diseases. Thus this protein may be involved in autoimmunity. Since the novel protein of invention has a Collagen triple helix repeat domain, it is likely that this protein may be involved in collagen related disorders and processes such as but not limited to osteogenesis, rheumatoid arthritis and osteoarthritis.

Finally, presence of somatotropin-like domain in the novel protein of invention suggests that it may have somatotropin (growth hormone) like function and behave as a growth hormone and be useful in control of growh and development/differentiation related functions such as but not limited maturation, lactation and puberty. Because of the involvement of growth hormone in many different physiologic functions, the novel protein may be involved in causing osteoporosis, obesity, aging and reproductive malfunction and hence could be used in treatment and/or diagnosis of these disorders.

The NOV11 nucleic acid, or fragments thereof, may further be useful in diagnostic applications, wherein the presence or amount of the nucleic acid or the protein are to be assessed.

NOV11 nucleic acids and polypeptides are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. For example the disclosed NOV11 protein have multiple hydrophilic regions, each of which can be used as an immunogen. In one embodiment, contemplated NOV11 epitope is from about amino acids 20 to 120. In another embodiment, the comtemplated NOV11 epitope is from about amino acids 130 to 150. In further embodiments, the contemplated NOV 11 epitope is from about amino acids 170 to 210, or from about amino acids 220 to 240. This novel protein also has value in development of powerful assay system for functional analysis of various human disorders, which will help in understanding of pathology of the disease and development of new drug targets for various disorders.

NOV12

A disclosed NOV12 nucleic acid of 5895 nucleotides (also referred to as SC132340676_A) encoding an plexin-1-like protein is shown in Table 12A. An open reading frame was identified beginning with an ATG initiation codon at nucleotides 77-79 and ending with a TGA codon at nucleotides 5798-5800. The putative untranslated regions are underlined and are upstream from the initiation codon and downstream from the termination codon in Table 12A. The start and stop codons are in bold letters.

TABLE 12A
NOV12 nucleotide sequence.

(SEQ ID NO:31)

CAGGGCTGAAGCTCCTGGCACCATGATGCTCACCCCAGCAGGACCAGAGCACCGAGGCCCAAGGCCCCAGCCTGCCATGC
CGCTGCCACCGCGGAGCCTGCAGGTGCTCCTGCTGCTGCTGCTGTTGCTGCTGCTGCTGCCGGGCATGTGGGCTGAGGCA
GGCTTGCCCAGGGCAGGCGGGGGTTCACAGCCCCCCTTCCGCACCTTCTCGGCCAGCGACTGGGGCCTCACCCACCTAGT
GGTGCATGAGCAGACAGGCGAGGTGTATGTGGGCGCAGTGAACCGCATCTATAAGCTGTCGGGGAACCTGACACTGCTGC
GGGCCCACGTCACGGGCCCTGTGGAGGACAACGAGAAGTGCTACCCGCCGCCCAGCGTGCAGTCCTGCCCCCACGGCCTG
GGCAGTACTGACAACGTCAACAAGCTGCTGCTGCTGGACTATGCCGCTAACCGCCTGCTGGCCTGTGGCAGCGCCTCCCA
GGGCATCTGCCAGTTCCTGCGTCTGGACGATCTCTTCAAACTGGGTGAGCCACACCACCGTAAGGAGCACTACCTGTCCA
GCGTGCAGGAGGCAGGCAGCATGGCGGGCGTGCTCATTGCCGGGCCACCGGGCCAGGGCCAGGCCAAGCTCTTCGTGGGC
ACACCCATCGATGGCAAGTCCGAGTACTTCCCCACACTGTCCAGCCGTCGGCTCATGGCCAACGAGGAGGATGCCGACAT
GTTCGGCTTCGTGTACCAGGATGAGTTTGTGTCATCACAGCTCAAGATCCCTTCGGACACGCTGTCCAAGTTCCCGGCCT
TTGACATCTACTATGTGTACAGCTTCCGCAGCGAGCAGTTTGTCTACTACCTCACGCTGCAGCTAGACACACAGCTGACC
TCGCCTGATGCCGCCGGCGAGCACTTCTTCACGTCCAAGATCGTGCGGCTCTGTGTGGACGACCCCAAATTCTACTCGTA
CGTTGAGTTCCCCATTGGCTGCGAGCAGGCGGGTGTGGAGTACCGCCTGGTGCAGGATGCCTACCTGAGCCGGCCCGGCC
GTGCCCTGGCCCACCAGCTGGGCCTGGCTGAGGACGAGGACGTGCTGTTCACTGTGTTCGCCCAGGGCCAGAAGAACCGC
GTGAAGCCACCAAAGGAGTCAGCACTGTGCCTGTTCACGCTCAGGGCCATCAAGGAGAAGATTAAGGAGCGCATCCAGTC
CTGCTACCGTGGTGAGGGCAAGCTCTCCCTGCCGTGGCTGCTCAACAAGGAGCTGGGCTGCATCAACTCGCCCCTGCAGA
TCGATGACGACTTCTGCGGGCAGGACTTCAACCAGCCCCTGGGGGGCACAGTCACCATTGAGGGGACGCCCCTGTTCGTG
GACAAGGATGATGGCCTGACCGCCGTGGCTGCCTATGACTATCGGGGCCGCACTGTGGTATTCGCCGGCACGCGAAGTGG
CCGCATCCGCAAGATCCTGGTGGACCTCTCAAACCCCGGTGGCCGGCCTGCCCTGGCCTACGAGAGCGTCGTGGCCCAGG
AGGGCAGCCCCATCCTGCGAGACCTCGTCCTCAGCCCCAACCACCAGTACCTCTACGCCATGACCGAGAAGCAGGTGACG
CGGGTGCCTGTGGAGAGCTGTGTGCAGTACACGTCCTGTGAGCTGTGTCTGGGGTCACGGGACCCCCACTGTGGCTGGTG
TGTCCTGCACAGCATGTGCTCGCGGCGGGACGCCTGTGAGCGAGCAGACGAGCCCCAGCGCTTTGCTGCGGACCTGCTGC
AGTGTGTGCAGCTGACTGTGCAGCCCCGCAATGTGTCTGTCACCATGTCCCAGGTCCCAGTACTTGTGCTGCAGGCCTGG
AACGTGCCTGACCTCTCAGCTGGCGTCAACTGCTCCTTCGAGGACTTCACGGAATCTGAGAGCGTCCTGGAGGATGGCCG
GATCCACTGCCGCTCACCCTCCGCCCGGGAGGTGGCGCCCATCACGCGGGGCCAGGGTGAGGGAGACCAGCGGGTGGTGA
AACTCTACCTAAAGTCCAAGGAGACAGGGAAGAAGTTTGCGTCTGTGGACTTCGTCTTCTACAACTGCAGCGTCCACCAG
TCGAGCTGCCTGTCCTGTGTCAACGGCTCCTTTCCCTGCCACTGGTGCAAATACCGCCACGTGTGCACACACAACGTGGC
TGACTGCCCCTTCCTGGAGGGCCGTGTCAACGTGTCTGAGGACTGCCCACAGATCCTGCCCTCCACGCAGATCTACGTGC
CAGTGGGATTCGTAAAACCCATCACCCTGGCCGCACGGAACCTGCCACAGCCACAGTCAGGCCAGCGTGGATATGAGTGC
CTCTTCCACATCCCGGGCAGCCCGGCCCGTGTCACCGCCCTGCGCTTCAACAGCTCCAGCCTGCAGTGCCAGAATTCCTC
GTACTCCTACGAGGGGAACGATGTCAGCGACCTGCCAGTGAACCTGTCAGTCGTGTGGAACGGCAACTTTGTCATTGACA
ACCCACAGAACATCCAGGCGCACCTCTACAAGTGCCCGGCCCTGCGCGAGAGCTGCGGCCTCTGCCTCAAGGCCGACCCG
CGCTTCGAGTGCGGATGGTGCGTGGCCGAGCGCCGCTGCTCCCTGCGACACCACTGCGCTGCCGACACACCTGCATCGTG
GATGCACGCGCGTCACGGCAGCAGTCGCTGCACCGACCCCAAGATCCTCAAGCTGTCCCCCGAGACGGGCCCGAGGCAGG
GCGGCACGCGGCTCACTATCACAGGCGAGAACCTGGGCCTGCGATTCGAAGACGTGCGTCTGGGCGTGCGCGTGGGCAAG
GTGCTGTGCAGCCCTGTGGAGAGCGAGTACATCAGTGCGGAGCAGATCGTCTGTGAGATCGGGGACGCCAGCTCCGTGCG
TGCCCATGACGCCCTGGTGGAGGTGTGTGTGCGGGACTGCTCACCACACTACCGCGCCCTGTCACCCAAGCGCTTCACCT
TCGTGACACCAACCTTCTACCGTGTGAGCCCCTCCCGTGGGCCTCTGTCAGGGGGCACCTGGATTGGCATCGAGGGAAGC
CACCTGAACGCAGGCAGTGATGTGGCTGTGTCGGTCGGTGGCCGGCCCTGCTCCTTCTCCTGGTCCAGGAGGAACTCCCG
TGAGATCCGGTGCCTGACACCCCCCGGGCAGAGCCCTGGCAGCGCTCCCATCATCATCAACATCAACCGCGCCCAGCTCA
CCAACCCTGAGGTGAAGTACAACTACACCGAGGACCCCACCATCCTGAGGATCGACCCCGAGTGGAGCATCAACAGCGGT
GGGACCCTCCTGACGGTCACAGGCACCAACCTGGCCACTGTCCGTGAACCCCGAATCCGGGCCAAGTATGGAGGCATTGA
GAGGGAGAAGTCCCTGGTGTACAATGACACCACCATGGTATGCCGCGCCCCGTCTGTGGCCAACCCTGTGCGCAGCCCAC
CAGAGCTGCGGGAGCGGCCGGATGAGCTGGGCTTCGTCATGGACAACGTGCGCTCCCTGCTTGTGCTCAACTCCACCTCC
TTCCTCTACTACCCTGACCCCGTACTGGAGCCACTCAGCCCCACTGGCCTGCTGGAGCTGAAGCCCAGCTCCCCACTCAT
CCTCAAGGGCCGGAACCTCTTGCCACCTGCACCCGGCAACTCCCGACTCAACTACACGGTGCTCATCGGCTCCACACCCT
GTACCCTCACCGTGTCGGAGACGCAACTGCTGTGCGAGGCGCCCAACCTCACTGGGCAGCACAAGGTCACGGTGCGTGCA
GGTGGCTTCGAGTTCTCGCCAGGGACACTGCAGGTGTACTCGGACAGCCTGCTGACGCTGCCTGCCATTGTGGGCATTGG
CGGAGGCGGGGGTCTCCTGCTGCTGGTCATCGTGGCTGTGCTCATCGCCTACAAGCGCAAGTCACGAGATGCTGACCGCA
CACTCAAGCGGCTGCAGCTCCAGATGGACAACCTGGAGTCCCGCGTGGCCCTCGAATGCAAGGAAGCCTTTGCAGAGCTG
CAGACAGACATCCACGAGCTGACCAATGACCTGGACGGTGCCGGCATCCCCTTCCTTGACTACCGGACATATGCCATGCG
GGTGCTCTTTCCTGGGATCGAGGACCACCCTGTGCTCAAGGAGATGGAGGTACAGGCCAATGTGGAGAAGTCGCTGACAC
TGTTCGGGCAGCTGCTGACCAAGAAGCACTTCCTGCTGACCTTCATCCGCACGCTGGAGGCACAGCGCAGCTTCTCCATG
CGCGACCGCGGGAATGTGGCCTCGCTCATCATGACGGCCCTGCAGGGCGAGATGGAATACGCCACAGGCGTGCTCAAGCA
GCTGCTTTCCGACCTCATCGAGAAGAACCTGGAGAGCAAGAACCACCCCAAGCTGCTACTGCGCCGGCCAACTGAGTCGG
TGGCAGAGAAGATGCTAACTAACTGGTTCACCTTCCTCTTGTATAAGTTCCTCAAGGAGTGCGCTGGGGAGCCGCTGTTC
ATGCTGTACTGCGCCATCAAGCAGCAGATGGAGAAGGGCCCCATTGACGCCATCACGGGTGAGGCACGCTACTCCCTGAG
TGAGGACAAGCTCATCCGGCAGCAGATTGACTACAAGACACTGACCCTGAACTGTGTGAACCCTGAGAATGAGAATGCAC
CTGAGGTGCCGGTGAAGGGGCTGGACTGTGACACGGTCACCCAGGCCAAGGAGAAGCTGCTGGACGCTGCCTACAAGGGC
GTGCCCTACTCCCAGCGGCCCAAGGCCGCGGACATGGACCTGGAGTGGCGCCAGGGCCGCATGGCGCGCATCATCCTGCA
GGACGAGGACGTCACCACCAAGATTGACAACGATTGGAAGAGGCTGAACACACTGGCTCACTACCAGGTGACAGACGGGT
CCTCGGTGGCACTGGTGCCCAAGCAGACGTCCGCCTACAACATCTCCAACTCCTCCACCTTCACCAAGTCCCTCAGCAGA
TACGAGAGCATGCTGCGCACGGCCAGCAGCCCCGACAGCCTGCGCTCGCGCACGCCCATGATCACGCCCGACCTGGAGAG
CGGCACCAAGCTGTGGCACCTGGTGAAGAACCACGACCACCTGGACCAGCGTGAGGGTGACCGCGGCAGCAAGATGGTCT
CGGAGATCTACTTGACACGGCTACTGGCCACCAAGCAGGGCACACTGCAGAAGTTTGTGGACGACCTGTTTGAGACCATC
TTCAGCACGGCACACCGGGGCTCAGCCCTGCCGCTGGCCATCAAGTACATGTTCGACTTCCTGGATGAGCAGGCCGACAA
GCACCAGATCCACGATGCTGACGTGCGCCACACCTGGAAGAGCAACTGCAGCCTGCCCCTGCGCTTCTGGGTGAACGTGA
TCAAGAACCCACAGTTTGTGTTCGACATTCACAAGAACAGCATCACGGACGCCTGCTTGTCGGTGGTGGCCCAGACCTTC
ATGGACTCCTGCTCCACCTCTGAGCACAAGCTGGGCAAGGACTCACCCTCCAACAAGCTGCTCTACGCCAAGGACATCCC
CAACTACAAGAGCTGGGTGGAGAGGAGGTACTATGCAGACATCGCCAAGATGCCAGCCATCAGCGACCAGGACATGAGTG
CGTATCTGGCTGAGCAGTCCCGCCTGCACCTGAGCCAGTTCAACAGCATGAGCGCCTTGCACGAGATCATCTCCTACATC
ACCAAGTACAAGGATGAGGTGCAGATCCTGGCAGCCCTGGAGAAGGATGAGCAGGCGCGGCGGCAGCGGCTGCGGAGCAA
GCTGGAGCAGGTGGTGGACACGATGGCCCTGAGCAGCTGAGCCCCAGCTGTGATCATCCAGCATGATGCAGCGTGAGGAC
AGCTGAGCAGGGACCGGGACAGCCCTCACCGCATGCGTGTGGAGTGTCCGGTGGT

In a search of public sequence databases, the NOV12 nucleic acid sequence, located on chromsome 8 has 2950 of 3362 bases (87%) identical to a plexin-1 mRNA from Mus musculus, (GENBANK-ID: D86948) (E=0.0). Public nucleotide databases include all GenBank databases and the GeneSeq patent database.

The disclosed NOV12 polypeptide (SEQ ID NO: 32) encoded by SEQ ID NO: 3,1 has 1925 amino acid residues and is presented in Table 12B using the one-letter amino acid code. Signal P. Psort and/or Hydropathy results predict that NOV12 contains a signal peptide and is likely to be localized in the plasma membrane with a certainty of 0.6000. In other embodiments, NOV12 is likely to be localized to the Golgi body with a certainty of 0.4000, to the endoplasmic reticulum (membrane) with a certainty of 0.1000, or to the endoplasmic reticulum (lumen) with a certainty of 0.1000. The most likely cleavage site for NOV12 is between positions 44 and 45: MWA-EA.

TABLE 12B
Encoded NOV12 protein sequence.

(SEQ ID NO:32)

MMLTPAGPEHRGPRPQPAMPLPPRSLQVLLLLLLLLLLLPGMWAEAGLPRAGGGSQPPFRTFSASDWGLTHL
VVHEQTGEVYVGAVNRIYKLSGNLTLLRAHVTGPVEDNEKCYPPPSVQSCPHGLGSTDNVNKLLLLDYAANR
LLACGSASQGICQFLRLDDLFKLGEPHHRKEHYLSSVQEAGSMAGVLIAGPPGQGQAKLFVGTPIDGKSEYF
PTLSSRRLMANEEDADMFGFVYQDEFVSSQLKIPSDTLSKFPAFDIYYVYSFRSEQFVYYLTLQLDTQLTSP
DAAGFHFFTSKIVRLCVDDPKFYSYVEEPIGCEQAGVEYRLVQDAYLSRPGRALAHQLGLAEDEDVLFTVFA
QGCRNRFKPPKESALCLFTLRAIKEKIKERIQSCYRGEGKLSLPWLLNKELGCINSPLQIDDDFCGQDFNQP
LGGTVTIEGTPLFVDKDDGLTAVAAYDYRGRTVVFAGTRSGRIRKILVDLSNPGGRPALAYESVVAQEGSPI
LRDLVLSPNHQYLYAMTEKQVTRVPVESCVQYTSCELCLGSRDPHCGWCVLHSMCSRRDACERADEPQRFAA
DLLQCVQLTVQPRNVSVTMSQVPVLVLQAWNVPDLSAGVNCSFEDFTESESVLEDGRIHCRSPSAREVAPIT
RGQGEGDQRVVKLYLKSKETGKKFASVDFVFYNCSVHQSSCLSCVNGSFPCHWCKYRHVCTHNVADCAFLEG
RVNVSEDCPQILPSTQIYVPVGVVKPITLAARNLPQPQSGQRGYECLFHIPGSPARVTALRFNSSSLQCQNS
SYSYEGNDVSDLPVNLSVVWNGNFVIDNPQNIQAHLYKCPALRESCGLCLKADPRFECGWCVAERRCSLRHH
CAADTPASWMHARHGSSRCTDPKILKLSPETGPRQGGTRLTITGENLGLRFEDVRLGVRVGKVLCSPVESEY
ISAEQIVCEIGDASSVRAHDALVEVCVRDCSPHYRALSPKRFTFVTPTFYRVSPSRGPLSGGTWIGIEGSHL
NAGSDVAVSVGGRPCSFSWSRRNSREIRCLTPPGQSPGSAPIIININRAQLTNPEVKYNYTEDPTILRIDPE
WSINSGGTLLTVTGTNLATVREPRIRAKYGGIERENCLVYNDTTMVCRAPSVANPVRSPPELGERPDELGFV
MDNVRSLLVLNSTSFLYYPDPVLEPLSPTGLLELKPSSPLILKGRNLLPPAPGNSRLNYTVLIGSTPCTLTV
SETQLLCEAPNLTGQHKVTVRAGGFEFSPGTLQVYSDSLLTLPAIVGIGGGGGLLLLVIVAVLIAYKRKSRD
ADRTLKRLQLQMDNLESRVALECKEAFAELQTDIHELTNDLDGAGIPFLDYRTYAMRVLFPGIEDHPVLKEM
EVQANVEKSLTLFGQLLTKKHFLLTFIRTLEAQRSFSMRDRGNVASLIMTALQGEMEYATGVLKQLLSDLIE
KNLESKNHPKLLLRRPTESVAEKMLTNWFTFLLYKFLKECAGEPLFMLYCAIKQQMEKGPIDAITGEARYSL
SEDKLIRQQIDYKTLTLNCVNPENENAPEVPVKGLDCDTVTQAKEKLLDAAYKGVPYSQRPKAADMDLEWRQ
GRMARIILQDEDVTTKIDNDWKRLNTLAHYQVTDGSSVALVPKQTSAYNISNSSTFTKSLSRYESMLRTASS
PDSLRSRTPMITPDLESGTKLWHLVKNHDHLDQREGDRGSKMVSEIYLTRLLATKQGTLQKFVDDLFETIFS
TAHRGSALPLAIKYMFDFLDEQADKHQIHDADVRHTWKSNCSLPLRFWVNVIKNPQFVFDIHKNSITDACLS
VVAQTFMDSCSTSEHKLGKDSPSNKLLYAKDIPNYKSWVERRYYADIAKMPAISDQDMSAYLAEQSRLHLSQ
FNSMSALHEIYSYITKYKDEVQILAALEKDEQARRQRLRSKLEQVVDTMALSS

A search of sequence databases reveals that the NOV12 amino acid sequence has 1820 of 1907 amino acid residues (95%) identical to, and 1859 of 1907 amino acid residues (97%) similar to, the 1894 amino acid residue plexin-1 protein from Mus musculus (P70206) (E=0.0). Public amino acid databases include the GenBank databases, SwissProt, PDB and PIR.

NOV12 is expressed in at least the following tissues: whole organism, brain, testis, trabecular Bone, lymph, germinal center B cells. In addition, NOV12 is predicted to be expressed in the following tissues because of the expression pattern of (GENBANK-ID: acc:AI255192) a closely related plexin-1 homolog in species Mus musculus: brain, testis.

The disclosed NOV12 polypeptide has homology to the amino acid sequences shown in the BLASTP data listed in Table 12C.

TABLE 12C
BLAST results for NOV12

| Gene Index/ | | Length | Identity | Positives | |
|---|---|---|---|---|---|
| Identifier | Protein/Organism | (aa) | (%) | (%) | Expect |
| Ptnr: SPTREMBL- | PLEXIN 1 - Mus | 1894 | 1820/1907 | 1859/1907 | 0.0 |
| ACC: P70206 | musculus | | (95%) | (97%) | |
| ptnr: SPTREMBL- | NOV/PLEXIN-A1 | 1754 | 1743/1762 | 1746/1762 | 0.0 |
| ACC: Q9UIW2 | PROTEIN - Homo | | (98%) | (99%) | |
| | sapiens | | | | |
| ptnr: SPTREMBL- | PLEXIN PRECURSOR - | 1905 | 1603/1893 | 1730/1893 | 0.0 |
| ACC: Q91823 | Xenopus laevis | | (84%) | (91%) | |
| ptnr: SWISSPROT- | Plexin A3 | 1871 | 1252/1874 | 1483/1874 | 0.0 |
| ACC: P51805 | precursor (Plexin | | (66%) | (79%) | |
| | 4) | | | | |
| ptnr: SPTREMBL- | PLEXIN 3 - Mus | 1872 | 1245/1874 | 1478/1874 | 0.0 |
| ACC: P70208 | musculus | | (66%) | (78%) | |

The homology between these and other sequences is shown graphically in the ClustalW analysis shown in Table 12D. In the ClustalW alignment of the NOV12 protein, as well as all other ClustalW analyses herein, the black outlined amino acid residues indicate regions of conserved sequence (i.e., regions that may be required to preserve structural or functional properties), whereas non-highlighted amino acid residues are less conserved and can potentially be altered to a much broader extent without altering protein structure or function.

[image]

[image]

Tables 12E-12N list the domain descriptions from DOMAIN analysis results against NOV12. This indicates that the NOV12 sequence has properties similar to those of other proteins known to contain this domain.

TABLE 12E
Domain Analysis of NOV12

gnl|Smart|smart00630, Sema, semaphorin domain (SEQ ID NO:113)
CD-Length = 430 residues, 100.0% aligned
Score = 242 bits (618), Expect = 1e−64

| | | | |
|---|---|---|---|
| Query: | 69 | LTHLVVHEQTGEVYVGAVNRIYKLSGNLTLLRAHVTGPVEDNEKCYPPPSVQSCPHGLGS | 128 |
| | | \| +\|++ \| \| +\|\|\|\| \|\|+\| \|\| \|\| \|\|\|\| + \| \| | |
| Sbjct: | 1 | LQNLLLDEDNGTLYVGARNRLYVLSLNLISEAEVKTGPVLSSPDCEEC--VSKGKDPP-- | 56 |
| Query: | 129 | TDNVNK-LLLLDYAANRLLACGS-ASQGICQFLRLDDLFKLGEPHHRKEHYLSSVQEAGS | 186 |
| | | \|\| \|\| \|\|\|\|\| \|+ \|\| \|\|+ \| \| +\|+ + \| +\| +\| \| + | |
| Sbjct: | 57 | TDCVNFIRLLLDYNADHLLVCGTNAFQPVCRLINLGNLDRL-EVGRESGRGRCPFDPQHN | 115 |
| Query: | 187 | MAGVLIAGPPGQGQAKLFVGTPID--GKSEYFPTLSSRRLMANEEDADMFGFVYQDEFVS | 244 |
| | | \|\|+ \| +\|+\|\|\| \| \| \| \| + \| | |
| Sbjct: | 116 | STAVLVDG-------ELYVGTVADFSGSDPAIYRSLSVRRLKGTSG-------PSLRTVL | 161 |
| Query: | 245 | SQLKIPSDTLSKFPAFDIYYVYSFRSEQFVYYLTLQLDTQLTSPDAAGEHFFTSKIVRLC | 304 |
| | | + + + +\|\|+\| \| \|\|\|+ + \| \|++ \|+\| | |
| Sbjct: | 162 | YDSRWLN---------EPNFVYAFESGDFVYF----FFRETAVEDENCGKAVVSRVARVC | 208 |
| Query: | 305 | VDD--------PKFYSYVEFPIGC---EQAGVEYRLVQDAYLSRPGRALAHQLGLAEDED | 353 |
| | | +\| \|+ \|+++ + \| + + +\| \|+\| \| +\| +\| | |
| Sbjct: | 209 | KNDVGGPRSLSKKWTSFLKARLECSVPGEFPFYFNELQAAFLLPAG---------SESDD | 259 |
| Query: | 354 | VLFTVFAQGQKNRVKPPKESALCLFTLRAIKEKIKERIQSCYRGEGKLSL----PWLLNK | 409 |
| | | \|\|+ \|\|+ \| \|\|+\| \|+\| \| \| + \| \| + + | |
| Sbjct: | 260 | VLYGVFSTS----SNPIPGSAVCAFSLSDINAVFNEPFKECETGNSQWLPYPRGLVPFPR | 315 |
| Query: | 410 | ELGCINSPLQI----DDDFC-GQDFNQPLGGTVTIEGTPLFV--DKDDGLTAVAA----Y | 458 |
| | | \| \|+\|\| \|\| + + \| \|\|\|\| \| + \|\|++\| | |
| Sbjct: | 316 | PGTCPNTPLSSKDLPDDVLNFIKTHPLMDEVVQPLTGRPLFVKTDSNYLLTSIAVDRVRT | 375 |
| Query: | 459 | DYRGRTVVFAGTRSGRIRKILVDLSNPGGRPALAYESVVAQEGSPILRDLVLSPNH | 514 |
| | | \| \|\|+\| \|\| \|\|\| \|+++ \|+ + \| \| \|\|\|+ \|\|\|\|\|\| | |
| Sbjct: | 376 | DGGNYTVLFLGTSDGRILKVVLSRSSSSSESVVLEEISVFDPGSPV-SDLVLSPKK | 430 |

TABLE 12F
Domain Analysis of NOV12

gnl|Pfam|pfam01403, Sema, Sema domain. The Sema domain occurs in
semaphorins, which are a large family of secreted and transmembrane
proteins, some of which function as repellent signals during axon
receptor. (SEQ ID NO:114)
CD-Length = 433 residues, 99.5% aligned
Score = 171 bits (432), Expect = 5e−43

| | | | |
|---|---|---|---|
| Query: | 69 | LTHLVVHEQTGEVYVGAVNRIYKLSGN----LTLLRAHVTGPVEDNEKCYPPPSVQSCPH | 124 |
| | | \|++ \| \| +\|\|\|\| \|\|+\| \|+ + \|+ \| \| \|+\| | |
| Sbjct: | 1 | FVTLLLDEDRGRLYVGARNRVYVLNLEDLSEVLNLKTGWPGSCETCEECNMKGKSP---- | 56 |
| Query: | 125 | GLGSTDNVN-KLLLLDYAANRLLACGS-ASQGICQFLRLDDLFKLGEPHHRKEHYLSSVQ | 182 |
| | | \|+ \| +\| \| \| \|\|+ \| \| +\| + \| \|\|\| \| + | |
| Sbjct: | 57 | ---LTECTNFIRVLQAYNDTHLYVCGTNAFQPVCTLINLGDLFSLDVDNEEDGCGDCPYD | 113 |
| Query: | 183 | EAGSMAGVLIAGPPGQGQAKLFVGTPIDGKSEYFPTLSSRRLMANEEDADMFGFVYQDEF | 242 |
| | | \|+ \|\|+ \| +\|+ \|\| \|\| + + \| + \| | |
| Sbjct: | 114 | PLGNTTSVLVQG------GELYSGTVID------FSGRDPSIRRLLGSHDGLRTEFHD-- | 159 |
| Query: | 243 | VSSQLKIPSDTLSKFPAFDIYYVYSFRSEQFVYYLTLQLDTQLTSPDAAGEHFFTSKIVR | 302 |
| | | \| \| +\|+ ++ \|+\|\|+\|\| \|+ \|\|+ + \|+ + \|++ \| | |
| Sbjct: | 160 | -SKWLNLPNFVD----SYPIHYVHSF-SDDKVYF----FFRETAVEDSNCKTIH-SRVAR | 208 |
| Query: | 303 | LCVDDPKFYSYVEFPIGC-------------EQAGVEYRLVQDAYLSRPGRALAHQLGLA | 349 |
| | | +\| +\|\| \|\|+\| \| + +\| \|++ \| \| | |
| Sbjct: | 209 | VCKNDPGGRSYLELNKWTTFLKARLNCSIPGEGTPFYFNELQAAFVLPTG---------A | 259 |
| Query: | 350 | EDEDVLFTVFAQGQKNRVKPPKESALCLFTLRAIKE--KIKERIQSCYRGEGKLSLPWLL | 407 |
| | | + + \|\|+ \|\| \|\|+\| \|++ \| + + + \|\| | |
| Sbjct: | 260 | DTDPVLYGVFTTS----SNSSAGSAVCAFSMSDINQVFEGPFKHQSPNSKWLPYRGKVPQ | 315 |
| Query: | 408 | NKELGCINSP-LQIDDDFCGQDFNQPLGGTVT--IEGTPLFVDKDDG--LTAVA-----A | 457 |
| | | + \| \|+ \| + \|\| \|\| \| + \|\|\|\| + \|\|++\| \| | |
| Sbjct: | 316 | PRPGQCPNASGLNLPDDTLNFIRCHPLMDEVVPPLHNVPLFVGQSGNYRLTSIAVDRVRA | 375 |
| Query: | 458 | YDYRGRTVVFAGTRSGRIRKILVDLSNPGGR---PALAYESVVAQEGSPILRDLVLS | 511 |
| | | \| + \|\|+\| \|\| \|\|+ \| \|\| + \|\|+\| +\| \|+ \| ++ \| | |
| Sbjct: | 376 | GDGQIYTVLFLGTDDGRV-LKQVVLSRSSSASYLVVVLEESLVFPDGEPVQRMVISS | 431 |

TABLE 12G
Domain Analysis of NOV12

gnl|Pfam|pfam01833, TIG, IPT TIG domain. This family consists of a
domain that has an immunoglobulin like fold. These domains are found
in cell surface receptors such as Met and Ron as well as in
intracellular transcription factors where it is involved in DNA
binding. (SEQ ID NO:115)
CD-Length = 85 residues, 100.0% aligned
Score = 78.2 bits (191), Expect = 4e−15

| | | | |
|---|---|---|---|
| Query: | 983 | PTFYRVSPSRGPLSGGTWIGIEGSHLNAGSDVAVSVGGRPCSFSWSRRNSREIRCLTPPG | 1042 |
| | | \| +\|\|\| \|\|\|\|\|\|\| \| \| \|\|+\| +\| \|+ \|+ \|\| \| + + +\| \| \|\|\| | |
| Sbjct: | 1 | PVITSISPSSGPLSGGTEITITGSNLGSGEDIKVTFGGTECDV--VSQEASQIVCKTPPY | 58 |
| Query: | 1043 | QSPGSAPIIININRAQLTNPEVKYNYT 1069 | |
| | | + \| \|+ ++++ \|++ \| + \| | |
| Sbjct: | 59 | ANGGPQPVTVSLDGGGLSSSPVTFTYV 85 | |

TABLE 12H
Domain Analysis of NOV12

gnl|Pfam|pfam01833, TIG, IPT TIG domain. This family consists of a
domain that has an immunoglobulin like fold. These domains are found
in cell surface receptors such as Met and Ron as well as in
intracellular transcription factors where it is involved in DNA
binding. (SEQ ID NO:115)
CD-Length = 85 residues, 100.0% aligned
Score = 60.1 bits (144), Expect = 1e−09

| | | | |
|---|---|---|---|
| Query: | 886 | PKILKLSPETGPRQGGTRLTITGENLGLRFEDVRLGVRVGKVLCSPVESEYISAEQIVCE | 945 |
| | | \| \| +\|\| +\|\| \|\|\| +\|\|\|\| \|\|\| + \| \| \| \| \| \|\|\|\|+ | |
| Sbjct: | 1 | PVITSISPSSGPLSGGTEITITGSNLGS---GEDIKVTFGGTECDVVSQEA---SQIVCK | 54 |
| Query: | 946 | IGDASSVRAHDALVEVCVRDCSPHYRALSPKRFTFV 981 | |
| | | ++ \| + \| \|\| \|\|+\| | |
| Sbjct: | 55 | TPPYANGGPQPVTVSLDGGGLSS-----SPVTFTYV 85 | |

TABLE 12I
Domain Analysis of NOV12

gnl|Pfam|pfam01833, TIG, IPT TIG domain. This family consists of a
domain that has an immunoglobulin like fold. These domains are found
in cell surface receptors such as Met and Ron as well as in
intracellular transcription factors where it is involved in DNA
binding. (SEQ ID NO:115)
CD-Length = 85 residues, 100.0% aligned
Score = 46.6 bits (109), Expect = 1e−05

| | | | |
|---|---|---|---|
| Query: | 1173 | PVLEPLSPTGLLELKPSSPLILKGRNLLPPAPGNSRLNYTVLIGSTPCTLT-VSETQLLC | 1231 |
| | | \|\|+ +\|\|+ \| + + + \| \|\| \| + \| \| \| \| + +\|++\| | |
| Sbjct: | 1 | PVITSISPSSG-PLSGGTEITITGSNL------GSGEDIKVTFGGTECDVVSQEASQIVC | 53 |
| Query: | 1232 | EAPNLTGQH----KVTVRAGGFEFSPGTLQVY 1259 | |
| | | + \| \|++ \|\| \|\| \| | |
| Sbjct: | 54 | KTPPYANGGPQPVTVSLDGGGLSSSPVTFTYV 85 | |

TABLE 12J
Domain Analysis of NOV12

gnl|Smart|smart00429, IPT, ig-like, plexins, transcription factors
(SEQ ID NO:116)
CD-Length = 93 residues, 100.0% aligned
Score = 70.9 bits (172), Expect = 6e−13

| | | | |
|---|---|---|---|
| Query: | 885 | DPKILKLSPETGPRQGGTRLTITGENLGLRFEDVRLGVRVGKVLCSPVESEYISAEQIVC | 944 |
| | | \|\| \| ++\|\| +\|\| \|\|\|\|+\|+ \|+\|\| \| + \| \|\|+\| \|+ + \|+ \| \|\|\| | |
| Sbjct: | 1 | DPVITRISPNSGPLSGGTRITLCGKNLDS-ISVVFVEVGVGEVPCTFLPSDV-SQTAIVC | 58 |
| Query: | 945 | EIGDASSVRAHDALVEVCVRDCSPHYRALSPKRFTFV 981 | |
| | | + \| \| \| + \| \|\|+\| | |
| Sbjct: | 59 | KTP-PYHNIPGSVPVRVEVGLRNGGVPG-EPSPFTYV 93 | |

TABLE 12
K. Domain Analysis of NOV 12

gn1|Pfam|pfam01437, Plexin_repeat, Plexin repeat. A cysteine rich
repeat found in several different extracellular receptors. The
function of the repeat is unknown. Three copies of the repeat are
found Plexin. Two copies of the repeat are found in mahogany
protein. A related C. elegans protein contains four copies of the
repeat. The Met receptor contains a single copy of the repeat.
The Pfam alignment shows 6 conserved cysteine residues that may
form three conserved disulphide bridges. (SEQ ID NO:117)
CD-Length = 48 residues, 100.0% aligned
Score = 59.3 bits (142), Expect = 2e−09

| | | | |
|---|---|---|---|
| Query: | 532 | SCVQYTSCELCLGSRDPHCGWCVLHSMCSRRDACERADEPQRFAADLLQCV | 582 |
| | | +\| \|+\|\|\| \|\| + \|\| \|\|\|\| \|+\| + \| \| + ++ \| | |
| Sbjct: | 1 | NCSQHTSCGSCLSAPDPGCGWCPSRKRCTRLEECSR---GEGWSQSQETCP | 48 |

TABLE 12L

Domain Analysis of NOV12

gn1|Pfam|pfam01437, Plexin_repeat, Plexin repeat. A cysteine rich
repeat found in several different extracellular receptors. The
function of the repeat is unknown. Three copies of the repeat are
found Plexin. Two copies of the repeat are found in mahogany
protein. A related C. elegans protein contains four copies of the
repeat. The Met receptor contains a single copy of the repeat.
The Pfam alignment shows 6 conserved cysteine residues that may
form three conserved disulphide bridges. (SEQ ID NO:117)
CD-Length = 48 residues, 100.0% aligned
Score = 53.5 bits (127), Expect = 1e−07

| | | | |
|---|---|---|---|
| Query: | 681 | NCSVHQSSCLSCVNGSFP-CHWCKYRHVCTHNVADCAFLEGRVNVSEDCP | 729 |
| | | \|\|\| \| \| \| \|\|++ \| \| \|\| \| \|\| +\|+ \|\| \| \|\| | |
| Sbjct: | 1 | NCSQHTS-CGSCLSAPDPGCGWCPSRKRCTRL-EECSRGEGWSQSQETCP | 48 |

TABLE 12M

Domain Analysis of NOV 12

gn1|Pfam|pfam01437, Plexin_repeat, Plexin repeat. A cysteine
rich repeat found in several different extracellular receptors.
The function of the repeat is unknown. Three copies of the
repeat are found Plexin. Two copies of the repeat are found in
mahogany protein. A related C. elegans protein contains four
copies of the repeat. The Met receptor contains a single copy
of the repeat. The Pfam alignment shows 6 conserved cysteine
residues that may form three conserved disulphide bridges.
(SEQ ID NO:117)
CD-Length = 48 residues, 89.6% aligned
Score =46.2 bits (108), Expect = 2e−05

| | | | |
|---|---|---|---|
| Query: | 835 | RESCGLCLKADPRFECGWCVAERRCSLRHHCAADTPASWMHARHGSSRC | 883 |
| | | \|\|\| \|\| \| \| \|\|\|\| + +\|\|+ \| + \| | |
| Sbjct: | 5 | HTSCGSCLSA-PDPGCGWCPSRKRCTRLEEC-----SRGEGWSQSQETC | 47 |

TABLE 12N
Domain Analysis of NOV12

gn1|Smart|smart00423, PSI, domain found in Plexins,
Semaphorins and Integrins (SEQ ID NO:118)
CD-Length = 47 residues, 89.4% aligned
Score = 44.3 bits (103), Expect = 6e−05

| | | | |
|---|---|---|---|
| Query: | 833 | ALRESCGLCLKADPRFECGWCVAERRCSLRHHCAADTPASWMHA | 876 |
| | | + \|\| \|\| \| + \| \|\| ++ \|\|+ \| + +\| | |
| Sbjct: | 3 | SAYTSCSECLLARDPY-CAWCSSQGRCTSGERCDS-LRQNWSSG | 44 |

Plexin is a type I membrane protein which was identified in Xenopus nervous system by hybridoma technique. Molecular cloning studies demonstrated that the extracellular segment of the plexin protein possesses three internal repeats of cysteine cluster which are homologous to the cysteine-rich domain of the c-met proto-oncogene protein product. A cell aggregation test revealed that the plexin protein mediated cell adhesion via a homophilic binding mechanism, in the presence of calcium ions. Plexin was expressed in the neuronal elements composing particular neuron circuits in Xenopus CNS and PNS. These findings indicate that plexin is a new member of the Ca(2+)-dependent cell adhesion molecules, and suggest that the molecule plays an important role in neuronal cell contact and neuron network formation.

In the developing nervous system axons navigate with great precision over large distances to reach their target areas. Chemorepulsive signals such as the semaphorins play an essential role in this process. The effects of one of these repulsive cues, semaphorin 3A (Sema3A), are mediated by the membrane protein neuropilin-1 (Npn-1). Recent work has shown that neuropilin-1 is essential but not sufficient to form functional Sema3A receptors and indicates that additional components are required to transduce signals from the cell surface to the cytoskeleton. Members of the plexin family interact with the neuropilins and act as co-receptors for Sema3A. Neuropilin/plexin interaction restricts the binding specificity of neuropilin-1 and allows the receptor complex to discriminate between two different semaphorins. Deletion of the highly conserved cytoplasmic domain of Plexin-A1 or -A2 creates a dominant negative Sema3A receptor that renders sensory axons resistant to the repulsive effects of Sema3A when expressed in sensory ganglia. These data suggest that functional semaphorin receptors contain plexins as signal-transducing and neuropilins as ligand-binding subunits.

Physiologic SEMA3A receptors consist of NRP1/PLXN1 complexes. Two semaphorin-binding proteins, plexin-1 (PLXN1) and neuropilin-1 (NRP1; 602069), form a stable complex. While SEMA3A binding to NRP1 does not alter nonneuronal cell morphology, SEMA3A interaction with NRP1/PLXN1 complexes induces adherent cells to round up. Expression of a dominant-negative PLXN1 in sensory neurons blocked SEMA3A-induced growth cone collapse. SEMA3A treatment led to the redistribution of growth cone NRP1 and PLXN1 into clusters.

The semaphorin family of proteins constitute one of the major cues for axonal guidance. The prototypic member of this family is Sema3A, previously designated semD/III or collapsin-1. Sema3A acts as a diffusible, repulsive guidance cue in vivo for the peripheral projections of embryonic dorsal root ganglion neurons. Sema3A binds with high affinity to neuropilin-1 on growth cone filopodial tips. Although neuropilin-1 is required for Sema3A action, it is incapable of transmitting a Sema3A signal to the growth cone interior. Instead, the Sema3A/neuropilin-1 complex interacts with another transmembrane protein, plexin, on the surface of growth cones. Certain semaphorins, other than Sema3A, can bind directly to plexins. The intracellular domain of plexin is responsible for initiating the signal transduction cascade leading to growth cone collapse, axon repulsion, or growth cone turning. This intracellular cascade involves the monomeric G-protein, Rac1, and a family of neuronal proteins, the CRMPs. Rac1 is likely to be involved in semaphorin-induced rearrangements of the actin cytoskeleton, but how plexin controls Rac1 activity is not known. Vertebrate CRMPs are homologous to the Caenorhabditis elegans unc-33 protein, which is required for proper axon morphology in worms. CRMPs are essential for Sema3A-induced, neuropilin-plexin-mediated growth cone collapse, but the molecular interactions of growth cone CRMPs are not well defined. Mechanistic aspects of plexin-based signaling for semaphorin guidance cues may have implications for other axon guidance events and for the basis of growth cone motility.

In Drosophila, plexin A is a functional receptor for semaphorin-1a. The human plexin gene family comprises at least nine members in four subfamilies. Plexin-B1 is a receptor for the transmembrane semaphorin Sema4D (CD100), and plexin-C1 is a receptor for the GPI-anchored semaphorin Sema7A (Sema-K1). Secreted (class 3) semaphorins do not bind directly to plexins, but rather plexins associate with neuropilins, coreceptors for these semaphorins. Plexins are widely expressed: in neurons, the expression of a truncated plexin-A1 protein blocks axon repulsion by Sema3A. The cytoplasmic domain of plexins associates with a tyrosine kinase activity. Plexins may also act as ligands mediating repulsion in epithelial cells in vitro. Thus, plexins are receptors for multiple (and perhaps all) classes of semaphorins, either alone or in combination with neuropilins, and trigger a novel signal transduction pathway controlling cell repulsion.

In addition, recent studies have identified semaphorins and their receptors as putative molecular cues involved in olfactory pathfinding, plasticity and regeneration. The semaphorins comprise a large family of secreted and transmembrane axon guidance proteins, being either repulsive or attractive in nature. Neuropilins were shown to serve as receptors for secreted class 3 semaphorins, whereas members of the plexin family are receptors for class 1 and V (viral) semaphorins.

The disclosed NOV12 nucleic acid of the invention encoding a Plexin-1-like protein includes the nucleic acid whose sequence is provided in Table 12A or a fragment thereof. The invention also includes a mutant or variant nucleic acid any of whose bases may be changed from the corresponding base shown in Table 12A while still encoding a protein that maintains its Plexin-1-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 29 percent of the bases may be so changed.

The disclosed NOV12 protein of the invention includes the Plexin-1-like protein whose sequence is provided in Table 12B. The invention also includes a mutant or variant protein any of whose residues may be changed from the corresponding residue shown in Table 12B, while still encoding a protein that maintains its Plexin-1-like activities and physiological functions, or a functional fragment thereof. In the mutant or variant protein, up to about 29 percent of the residues may be so changed.

The protein similarity information, expression pattern, and map location for the plexin-1-like protein and the NOV12 protein disclosed herein suggest that this plexin-1-like protein may have important structural and/or physiological functions characteristic of the mannosidase protein family. Therefore, the nucleic acids and proteins of the invention are useful in potential diagnostic and therapeutic applications and as a research tool. These applications include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo (vi) biological defense weapon.

The NOV12 nucleic acids and proteins of the invention are useful in potential diagnostic and therapeutic applications implicated in various diseases and disorders described below and/or other pathologies. For example, the compositions of the present invention will have efficacy for treatment of patients suffering from AIDS, cancer therapy, treatment of Neurologic diseases, Brain and/or autoimmune disorders like encephalomyelitis, neurodegenerative disorders, Alzheimer's Disease, Parkinson's Disorder, immune disorders, and hematopoietic disorders, endocrine diseases, muscle disorders, inflammation and wound repair, bacterial, fungal, protozoal and viral infections (particularly infections caused by HIV-1 or HIV-2), pain, cancer (including but not limited to Neoplasm; adenocarcinoma; lymphoma; prostate cancer; uterus cancer), anorexia, bulimia, asthma, Parkinson's disease, acute heart failure, hypotension, hypertension, urinary retention, osteoporosis, Crohn's disease; multiple sclerosis; and Treatment of Albright Hereditary Ostoeodystrophy, angina pectoris, myocardial infarction, ulcers, asthma, allergies, benign prostatic hypertrophy, and psychotic and neurological disorders, including anxiety, schizophrenia, manic depression, delirium, dementia, severe mental retardation and dyskinesias, such as Huntington's disease or Gilles de la Tourette syndrome, and/or other pathologies/disorders. The NOV12 nucleic acid, or fragments thereof, may further be useful in diagnostic applications, wherein the presence or amount of the nucleic acid or the protein are to be assessed.

NOV12 nucleic acids and polypeptides are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. For example the disclosed NOV12 protein have multiple hydrophilic regions, each of which can be used as an immunogen. This novel protein also has value in development of powerful assay system for functional analysis of various human disorders, which will help in understanding of pathology of the disease and development of new drug targets for various disorders.

NOVX Nucleic Acids and Polypeptides

One aspect of the invention pertains to isolated nucleic acid molecules that encode NOVX polypeptides or biologically active portions thereof. Also included in the invention are nucleic acid fragments sufficient for use as hybridization probes to identify NOVX-encoding nucleic acids (e.g., NOVX mRNAs) and fragments for use as PCR primers for the amplification and/or mutation of NOVX nucleic acid molecules. As used herein, the term “nucleic acid molecule” is intended to include DNA molecules (e.g., cDNA or genomic DNA), RNA molecules (e.g., mRNA), analogs of the DNA or RNA generated using nucleotide analogs, and derivatives, fragments and homologs thereof. The nucleic acid molecule may be single-stranded or double-stranded, but preferably is comprised double-stranded DNA.

An NOVX nucleic acid can encode a mature NOVX polypeptide. As used herein, a “mature” form of a polypeptide or protein disclosed in the present invention is the product of a naturally occurring polypeptide or precursor form or proprotein. The naturally occurring polypeptide, precursor or proprotein includes, by way of nonlimiting example, the full-length gene product, encoded by the corresponding gene. Alternatively, it may be defined as the polypeptide, precursor or proprotein encoded by an ORF described herein. The product “mature” form arises, again by way of nonlimiting example, as a result of one or more naturally occurring processing steps as they may take place within the cell, or host cell, in which the gene product arises. Examples of such processing steps leading to a “mature” form of a polypeptide or protein include the cleavage of the N-terminal methionine residue encoded by the initiation codon of an ORF, or the proteolytic cleavage of a signal peptide or leader sequence. Thus a mature form arising from a precursor polypeptide or protein that has residues 1 to N, where residue 1 is the N-terminal methionine, would have residues 2 through N remaining after removal of the N-terminal methionine. Alternatively, a mature form arising from a precursor polypeptide or protein having residues 1 to N, in which an N-terminal signal sequence from residue 1 to residue M is cleaved, would have the residues from residue M+1 to residue N remaining. Further as used herein, a “mature” form of a polypeptide or protein may arise from a step of post-translational modification other than a proteolytic cleavage event. Such additional processes include, by way of non-limiting example, glycosylation, myristoylation or phosphorylation. In general, a mature polypeptide or protein may result from the operation of only one of these processes, or a combination of any of them.

The term “probes”, as utilized herein, refers to nucleic acid sequences of variable length, preferably between at least about 10 nucleotides (nt), 100 nt, or as many as approximately, e.g., 6,000 nt, depending upon the specific use. Probes are used in the detection of identical, similar, or complementary nucleic acid sequences. Longer length probes are generally obtained from a natural or recombinant source, are highly specific, and much slower to hybridize than shorter-length oligomer probes. Probes may be single- or double-stranded and designed to have specificity in PCR, membrane-based hybridization technologies, or ELISA-like technologies.

The term “isolated” nucleic acid molecule, as utilized herein, is one, which is separated from other nucleic acid molecules which are present in the natural source of the nucleic acid. Preferably, an “isolated” nucleic acid is free of sequences which naturally flank the nucleic acid (i.e., sequences located at the 5′-and 3′-termini of the nucleic acid) in the genomic DNA of the organism from which the nucleic acid is derived. For example, in various embodiments, the isolated NOVX nucleic acid molecules can contain less than about 5 kb, 4 kb, 3 kb, 2 kb, 1 kb, 0.5 kb or 0.1 kb of nucleotide sequences which naturally flank the nucleic acid molecule in genomic DNA of the cell/tissue from which the nucleic acid is derived (e.g., brain, heart, liver, spleen, etc.). Moreover, an “isolated” nucleic acid molecule, such as a cDNA molecule, can be substantially free of other cellular material or culture medium when produced by recombinant techniques, or of chemical precursors or other chemicals when chemically synthesized.

A nucleic acid molecule of the invention, e.g., a nucleic acid molecule having the nucleotide sequence SEQ ID NOS: 1, 3, 5, 7, 9, 11, 13, 15, 17, 19, 21, 23, 25, 27, 29, and 31, or a complement of this aforementioned nucleotide sequence, can be isolated using standard molecular biology techniques and the sequence information provided herein. Using all or a portion of the nucleic acid sequence of SEQ ID NOS: 1, 3, 5, 7, 9, 11, 13, 15, 17, 19, 21, 23, 25, 27, 29, and 31 as a hybridization probe, NOVX molecules can be isolated using standard hybridization and cloning techniques (e.g., as described in Sambrook, et al., (eds.), MOLECULAR CLONING: A LABORATORY MANUAL 2^{nd} Ed., Cold Spring Harbor Laboratory Press, Cold Spring Harbor, N.Y., 1989; and Ausubel, et al., (eds.), CURRENT PROTOCOLS IN MOLECULAR BIOLOGY, John Wiley & Sons, New York, NY, 1993.) A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to NOVX nucleotide sequences can be prepared by standard synthetic techniques, e.g., using an automated DNA synthesizer.

As used herein, the term “oligonucleotide” refers to a series of linked nucleotide residues, which oligonucleotide has a sufficient number of nucleotide bases to be used in a PCR reaction. A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides comprise portions of a nucleic acid sequence having about 10 nt, 50 nt, or 100 nt in length, preferably about 15 nt to 30 nt in length. In one embodiment of the invention, an oligonucleotide comprising a nucleic acid molecule less than 100 nt in length would further comprise at least 6 contiguous nucleotides SEQ ID NOS: 1, 3, 5, 7, 9, 11, 13, 15, 17, 19, 21, 23, 25, 27, 29, and 31, or a complement thereof. Oligonucleotides may be chemically synthesized and may also be used as probes.

In another embodiment, an isolated nucleic acid molecule of the invention comprises a nucleic acid molecule that is a complement of the nucleotide sequence shown in SEQ ID NOS: 1, 3, 5, 7,9, 11, 13, 15, 17, 19, 21, 23, 25, 27, 29, and31, or a portion of this nucleotide sequence (e.g., a fragment that can be used as a probe or primer or a fragment encoding a biologically-active portion of an NOVX polypeptide). A nucleic acid molecule that is complementary to the nucleotide sequence shown SEQ ID NOS: 1, 3, 5, 7, 9, 11, 13, 15, 17, 19, 21, 23, 25, 27, 29, or 31 is one that is sufficiently complementary to the nucleotide sequence shown SEQ ID NOS: 1, 3, 5, 7, 9, 11, 13, 15, 17, 19, 21, 23, 25, 27, 29, or 31 that it can hydrogen bond with little or no mismatches to the nucleotide sequence shown SEQ ID NOS: 1, 3, 5, 7, 9, 11, 13, 15, 17, 19, 21, 23, 25, 27, 29, and 31, thereby forming a stable duplex.

As used herein, the term “complementary” refers to Watson-Crick or Hoogsteen base pairing between nucleotides units of a nucleic acid molecule, and the term “binding” means the physical or chemical interaction between two polypeptides or compounds or associated polypeptides or compounds or combinations thereof. Binding includes ionic, non-ionic, van der Waals, hydrophobic interactions, and the like. A physical interaction can be either direct or indirect. Indirect interactions may be through or due to the effects of another polypeptide or compound. Direct binding refers to interactions that do not take place through, or due to, the effect of another polypeptide or compound, but instead are without other substantial chemical intermediates.

Fragments provided herein are defined as sequences of at least 6 (contiguous) nucleic acids or at least 4 (contiguous) amino acids, a length sufficient to allow for specific hybridization in the case of nucleic acids or for specific recognition of an epitope in the case of amino acids, respectively, and are at most some portion less than a full length sequence. Fragments may be derived from any contiguous portion of a nucleic acid or amino acid sequence of choice. Derivatives are nucleic acid sequences or amino acid sequences formed from the native compounds either directly or by modification or partial substitution. Analogs are nucleic acid sequences or amino acid sequences that have a structure similar to, but not identical to, the native compound but differs from it in respect to certain components or side chains. Analogs may be synthetic or from a different evolutionary origin and may have a similar or opposite metabolic activity compared to wild type. Homologs are nucleic acid sequences or amino acid sequences of a particular gene that are derived from different species.

Derivatives and analogs may be full length or other than full length, if the derivative or analog contains a modified nucleic acid or amino acid, as described below. Derivatives or analogs of the nucleic acids or proteins of the invention include, but are not limited to, molecules comprising regions that are substantially homologous to the nucleic acids or proteins of the invention, in various embodiments, by at least about 70%, 80%, or 95% identity (with a preferred identity of 80-95%) over a nucleic acid or amino acid sequence of identical size or when compared to an aligned sequence in which the alignment is done by a computer homology program known in the art, or whose encoding nucleic acid is capable of hybridizing to the complement of a sequence encoding the aforementioned proteins under stringent, moderately stringent, or low stringent conditions. See e.g. Ausubel, et al., CURRENT PROTOCOLS IN MOLECULAR BIOLOGY, John Wiley & Sons, New York, NY, 1993, and below.

A “homologous nucleic acid sequence” or “homologous amino acid sequence,” or variations thereof, refer to sequences characterized by a homology at the nucleotide level or amino acid level as discussed above. Homologous nucleotide sequences encode those sequences coding for isoforms of NOVX polypeptides. Isoforms can be expressed in different tissues of the same organism as a result of, for example, alternative splicing of RNA. Alternatively, isoforms can be encoded by different genes. In the invention, homologous nucleotide sequences include nucleotide sequences encoding for an NOVX polypeptide of species other than humans, including, but not limited to: vertebrates, and thus can include, e.g., frog, mouse, rat, rabbit, dog, cat cow, horse, and other organisms. Homologous nucleotide sequences also include, but are not limited to, naturally occurring allelic variations and mutations of the nucleotide sequences set forth herein. A homologous nucleotide sequence does not, however, include the exact nucleotide sequence encoding human NOVX protein. Homologous nucleic acid sequences include those nucleic acid sequences that encode conservative amino acid substitutions (see below) in SEQ ID NOS: 1, 3, 5, 7, 9, 11, 13, 15, 17, 19, 21, 23, 25, 27, 29, and 31, as well as a polypeptide possessing NOVX biological activity. Various biological activities of the NOVX proteins are described below.

An NOVX polypeptide is encoded by the open reading frame (“ORF”) of an NOVX nucleic acid. An ORF corresponds to a nucleotide sequence that could potentially be translated into a polypeptide. A stretch of nucleic acids comprising an ORF is uninterrupted by a stop codon. An ORF that represents the coding sequence for a full protein begins with an ATG “start” codon and terminates with one of the three “stop” codons, namely, TAA, TAG, or TGA. For the purposes of this invention, an ORF may be any part of a coding sequence, with or without a start codon, a stop codon, or both. For an ORF to be considered as a good candidate for coding for a bona fide cellular protein, a minimum size requirement is often set, e.g., a stretch of DNA that would encode a protein of 50 amino acids or more.

The nucleotide sequences determined from the cloning of the human NOVX genes allows for the generation of probes and primers designed for use in identifying and/or cloning NOVX homologues in other cell types, e.g. from other tissues, as well as NOVX homologues from other vertebrates. The probe/primer typically comprises substantially purified oligonucleotide. The oligonucleotide typically comprises a region of nucleotide sequence that hybridizes under stringent conditions to at least about 12, 25, 50, 100, 150, 200, 250, 300, 350 or 400 consecutive sense strand nucleotide sequence SEQ ID NOS: 1, 3, 5, 7, 9, 11, 13, 15, 17, 19, 21, 23, 25, 27, 29, or 31; or an anti-sense strand nucleotide sequence of SEQ ID NOS: 1, 3, 5, 7, 9, 11, 13, 15, 17, 19, 21, 23, 25, 27, 29, or 31; or of a naturally occurring mutant of SEQ ID NOS: 1, 3, 5, 7, 9, 11, 13, 15, 17, 19, 21, 23, 25, 27, 29, and 31.

Probes based on the human NOVX nucleotide sequences can be used to detect transcripts or genomic sequences encoding the same or homologous proteins. In various embodiments, the probe further comprises a label group attached thereto, e.g. the label group can be a radioisotope, a fluorescent compound, an enzyme, or an enzyme co-factor. Such probes can be used as a part of a diagnostic test kit for identifying cells or tissues which mis-express an NOVX protein, such as by measuring a level of an NOVX-encoding nucleic acid in a sample of cells from a subject e.g., detecting NOVX mRNA levels or determining whether a genomic NOVX gene has been mutated or deleted. “A polypeptide having a biologically-active portion of an NOVX polypeptide” refers to polypeptides exhibiting activity similar, but not necessarily identical to, an activity of a polypeptide of the invention, including mature forms, as measured in a particular biological assay, with or without dose dependency. A nucleic acid fragment encoding a “biologically-active portion of NOVX” can be prepared by isolating a portion SEQ ID NOS: 1, 3, 5, 7, 9, 11, 13, 15, 17, 19, 21, 23, 25, 27, 29, or 31, that encodes a polypeptide having an NOVX biological activity (the biological activities of the NOVX proteins are described below), expressing the encoded portion of NOVX protein (e.g., by recombinant expression in vitro) and assessing the activity of the encoded portion of NOVX.

NOVX Nucleic Acid and Polypeptide Variants

The invention further encompasses nucleic acid molecules that differ from the nucleotide sequences shown in SEQ ID NOS: 1, 3, 5, 7, 9, 11, 13, 15, 17, 19, 21, 23, 25, 27, 29, and 31 due to degeneracy of the genetic code and thus encode the same NOVX proteins as that encoded by the nucleotide sequences shown in SEQ ID NOS: 1, 3, 5, 7, 9, 11, 13, 15, 17, 19, 21, 23, 25, 27, 29, and 31. In another embodiment, an isolated nucleic acid molecule of the invention has a nucleotide sequence encoding a protein having an amino acid sequence shown in SEQ ID NOS: 2, 4, 6, 8, 10, 12, 14, 16, 18, 20, 22, 24, 26, 28, 30, or 32.

In addition to the human NOVX nucleotide sequences shown in SEQ ID NOS: 1, 3, 5, 7, 9, 11, 13, 15, 17, 19, 21, 23, 25, 27, 29, and 31, it will be appreciated by those skilled in the art that DNA sequence polymorphisms that lead to changes in the amino acid sequences of the NOVX polypeptides may exist within a population (e.g., the human population). Such genetic polymorphism in the NOVX genes may exist among individuals within a population due to natural allelic variation. As used herein, the terms “gene” and “recombinant gene” refer to nucleic acid molecules comprising an open reading frame (ORF) encoding an NOVX protein, preferably a vertebrate NOVX protein. Such natural allelic variations can typically result in 1-5% variance in the nucleotide sequence of the NOVX genes. Any and all such nucleotide variations and resulting amino acid polymorphisms in the NOVX polypeptides, which are the result of natural allelic variation and that do not alter the functional activity of the NOVX polypeptides, are intended to be within the scope of the invention.

Moreover, nucleic acid molecules encoding NOVX proteins from other species, and thus that have a nucleotide sequence that differs from the human SEQ ID NOS: 1, 3, 5, 7, 9, 11, 13, 15, 17, 19, 21, 23, 25, 27, 29, and 31 are intended to be within the scope of the invention. Nucleic acid molecules corresponding to natural allelic variants and homologues of the NOVX cDNAs of the invention can be isolated based on their homology to the human NOVX nucleic acids disclosed herein using the human cDNAs, or a portion thereof, as a hybridization probe according to standard hybridization techniques under stringent hybridization conditions.

Accordingly, in another embodiment, an isolated nucleic acid molecule of the invention is at least 6 nucleotides in length and hybridizes under stringent conditions to the nucleic acid molecule comprising the nucleotide sequence of SEQ ID NOS: 1, 3, 5, 7, 9, 11, 13, 15, 17, 19, 21, 23, 25, 27, 29, and 31. In another embodiment, the nucleic acid is at least 10, 25, 50, 100, 250, 500, 750, 1000, 1500, or 2000 or more nucleotides in length. In yet another embodiment, an isolated nucleic acid molecule of the invention hybridizes to the coding region. As used herein, the term “hybridizes under stringent conditions” is intended to describe conditions for hybridization and washing under which nucleotide sequences at least 60% homologous to each other typically remain hybridized to each other.

Homologs (i.e., nucleic acids encoding NOVX proteins derived from species other than human) or other related sequences (e.g., paralogs) can be obtained by low, moderate or high stringency hybridization with all or a portion of the particular human sequence as a probe using methods well known in the art for nucleic acid hybridization and cloning.

As used herein, the phrase “stringent hybridization conditions” refers to conditions under which a probe, primer or oligonucleotide will hybridize to its target sequence, but to no other sequences. Stringent conditions are sequence-dependent and will be different in different circumstances. Longer sequences hybridize specifically at higher temperatures than shorter sequences. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e.g., 10 nt to 50 nt) and at least about 60° C. for longer probes, primers and oligonucleotides. Stringent conditions may also be achieved with the addition of destabilizing agents, such as formamide.

Stringent conditions are known to those skilled in the art and can be found in Ausubel, et al., (eds.), CURRENT PROTOCOLS IN MOLECULAR BIOLOGY, John Wiley & Sons, N.Y. (1989), 6.3.1-6.3.6. Preferably, the conditions are such that sequences at least about 65%, 70%, 75%, 85%, 90%, 95%, 98%, or 99% homologous to each other typically remain hybridized to each other. A non-limiting example of stringent hybridization conditions are hybridization in a high salt buffer comprising 6×SSC, 50 mM Tris-HCI (pH 7.5), 1 mM EDTA, 0.02% PVP, 0.02% Ficoll, 0.02% BSA, and 500 mg/ml denatured salmon sperm DNA at 65° C., followed by one or more washes in 0.2×SSC, 0.01% BSA at 50° C. An isolated nucleic acid molecule of the invention that hybridizes under stringent conditions to the sequences SEQ ID NOS: 1, 3, 5, 7, 9, 11, 13, 15, 17, 19, 21, 23, 25, 27, 29, and 31, corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

In a second embodiment, a nucleic acid sequence that is hybridizable to the nucleic acid molecule comprising the nucleotide sequence of SEQ ID NOS: 1, 3, 5, 7, 9, 11, 13, 15, 17, 19, 21, 23, 25, 27, 29, and 31, or fragments, analogs or derivatives thereof, under conditions of moderate stringency is provided. A non-limiting example of moderate stringency hybridization conditions are hybridization in 6×SSC, 5×Denhardt's solution, 0.5% SDS and 100 mg/ml denatured salmon sperm DNA at 55° C., followed by one or more washes in 1×SSC, 0.1% SDS at 37° C. Other conditions of moderate stringency that may be used are well-known within the art. See, e.g., Ausubel, et al. (eds.), 1993, CURRENT PROTOCOLS IN MOLECULAR BIOLOGY, John Wiley & Sons, NY, and Kriegler, 1990; GENE TRANSFER AND EXPRESSION, A LABORATORY MANUAL, Stockton Press, NY.

In a third embodiment, a nucleic acid that is hybridizable to the nucleic acid molecule comprising the nucleotide sequences SEQ ID NOS: 1, 3, 5, 7, 9, 11, 13, 15, 17, 19, 21, 23, 25, 27, 29, and 31, or fragments, analogs or derivatives thereof, under conditions of low stringency, is provided. A non-limiting example of low stringency hybridization conditions are hybridization in 35% formamide, 5×SSC, 50 mM Tris-HCl (pH 7.5), 5 mM EDTA, 0.02% PVP, 0.02% Ficoll, 0.2% BSA, 100 mg/ml denatured salmon sperm DNA, 10% (wt/vol) dextran sulfate at 40° C., followed by one or more washes in 2×SSC, 25 mM Tris-HCl (pH 7.4), 5 mM EDTA, and 0.1% SDS at 50° C. Other conditions of low stringency that may be used are well known in the art (e.g., as employed for cross-species hybridizations). See, e.g., Ausubel, et al. (eds.), 1993, CURRENT PROTOCOLS IN MOLECULAR BIOLOGY, John Wiley & Sons, NY, and Kriegler, 1990, GENE TRANSFER AND EXPRESSION, A LABORATORY MANUAL, Stockton Press, NY; Shilo and Weinberg, 1981. Proc Natl Acad Sci USA 78: 6789-6792.

Conservative Mutations

In addition to naturally-occurring allelic variants of NOVX sequences that may exist in the population, the skilled artisan will further appreciate that changes can be introduced by mutation into the nucleotide sequences SEQ ID NOS: 1, 3, 5, 7, 9, 11, 13, 15, 17, 19, 21, 23, 25, 27, 29, and 31, thereby leading to changes in the amino acid sequences of the encoded NOVX proteins, without altering the functional ability of said NOVX proteins. For example, nucleotide substitutions leading to amino acid substitutions at “non-essential” amino acid residues can be made in the sequence SEQ ID NOS: 2, 4, 6, 8, 10, 12, 14, 16, 18, 20, 22, 24, 26, 28, 30, or 32. A “non-essential” amino acid residue is a residue that can be altered from the wild-type sequences of the NOVX proteins without altering their biological activity, whereas an “essential” amino acid residue is required for such biological activity. For example, amino acid residues that are conserved among the NOVX proteins of the invention are predicted to be particularly non-amenable to alteration. Amino acids for which conservative substitutions can be made are well-known within the art.

Another aspect of the invention pertains to nucleic acid molecules encoding NOVX proteins that contain changes in amino acid residues that are not essential for activity. Such NOVX proteins differ in amino acid sequence from SEQ ID NOS: 1, 3, 5, 7,9, 11, 13, 15, 17, 19, 21, 23, 25, 27, 29, and 31 yet retain biological activity. In one embodiment, the isolated nucleic acid molecule comprises a nucleotide sequence encoding a protein, wherein the protein comprises an amino acid sequence at least about 45% homologous to the amino acid sequences SEQ ID NOS: 2, 4, 6, 8, 10, 12, 14, 16, 18, 20, 22, 24, 26, 28, 30, and 32. Preferably, the protein encoded by the nucleic acid molecule is at least about 60% homologous to SEQ ID NOS: 2, 4, 6, 8, 10, 12, 14, 16, 18, 20, 22, 24, 26, 28, 30, and 32; more preferably at least about 70% homologous SEQ ID NOS: 2, 4, 6, 8, 10, 12, 14, 16, 18, 20, 22, 24, 26, 28, 30, or 32; still more preferably at least about 80% homologous to SEQ ID NOS: 2, 4, 6, 8, 10, 12, 14, 16, 18, 20, 22, 24, 26, 28, 30, or 32; even more preferably at least about 90% homologous to SEQ ID NOS: 2, 4, 6, 8, 10, 12, 14, 16, 18, 20, 22, 24, 26, 28, 30, or 32; and most preferably at least about 95% homologous to SEQ ID NOS: 2, 4, 6, 8, 10, 12, 14, 16, 18, 20, 22, 24, 26, 28, 30, or 32.

An isolated nucleic acid molecule encoding an NOVX protein homologous to the protein of SEQ ID NOS: 2, 4, 6, 8, 10, 12, 14, 16, 18, 20, 22, 24, 26, 28, 30, or 32 can be created by introducing one or more nucleotide substitutions, additions or deletions into the nucleotide sequence of SEQ ID NOS: 1, 3, 5, 7, 9, 11, 13, 15, 17, 19, 21, 23, 25, 27, 29, and 31, such that one or more amino acid substitutions, additions or deletions are introduced into the encoded protein.

Mutations can be introduced into SEQ ID NOS: 1, 3, 5, 7, 9, 11, 13, 15, 17, 19, 21, 23, 25, 27, 29, and 31 by standard techniques, such as site-directed mutagenesis and PCR-mediated mutagenesis. Preferably, conservative amino acid substitutions are made at one or more predicted, non-essential amino acid residues. A “conservative amino acid substitution” is one in which the amino acid residue is replaced with an amino acid residue having a similar side chain. Families of amino acid residues having similar side chains have been defined within the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted non-essential amino acid residue in the NOVX protein is replaced with another amino acid residue from the same side chain family. Alternatively, in another embodiment, mutations can be introduced randomly along all or part of an NOVX coding sequence, such as by saturation mutagenesis, and the resultant mutants can be screened for NOVX biological activity to identify mutants that retain activity. Following mutagenesis SEQ ID NOS: 1, 3, 5, 7, 9, 11, 13, 15, 17, 19, 21, 23, 25, 27, 29, and 31, the encoded protein can be expressed by any recombinant technology known in the art and the activity of the protein can be determined.

The relatedness of amino acid families may also be determined based on side chain interactions. Substituted amino acids may be fully conserved “strong” residues or fully conserved “weak” residues. The “strong” group of conserved amino acid residues may be any one of the following groups: STA, NEQK, NHQK, NDEQ, QHRK, MILV, MILF, HY, FYW, wherein the single letter amino acid codes are grouped by those amino acids that may be substituted for each other. Likewise, the “weak” group of conserved residues may be any one of the following: CSA, ATV, SAG, STNK, STPA, SGND, SNDEQK, NDEQHK, NEQHRK, VLIM, HFY, wherein the letters within each group represent the single letter amino acid code.

In one embodiment, a mutant NOVX protein can be assayed for (i) the ability to form protein:protein interactions with other NOVX proteins, other cell-surface proteins, or biologically-active portions thereof, (ii) complex formation between a mutant NOVX protein and an NOVX ligand; or (iii) the ability of a mutant NOVX protein to bind to an intracellular target protein or biologically-active portion thereof; (e.g. avidin proteins).

In yet another embodiment, a mutant NOVX protein can be assayed for the ability to regulate a specific biological function (e.g., regulation of insulin release).

Antisense Nucleic Acids

Another aspect of the invention pertains to isolated antisense nucleic acid molecules that are hybridizable to or complementary to the nucleic acid molecule comprising the nucleotide sequence of SEQ ID NOS: 1, 3, 5, 7, 9, 11, 13, 15, 17, 19, 21, 23, 25, 27, 29, and 31, or fragments, analogs or derivatives thereof. An “antisense” nucleic acid comprises a nucleotide sequence that is complementary to a “sense” nucleic acid encoding a protein (e.g., complementary to the coding strand of a double-stranded cDNA molecule or complementary to an mRNA sequence). In specific aspects, antisense nucleic acid molecules are provided that comprise a sequence complementary to at least about 10, 25, 50, 100, 250 or 500 nucleotides or an entire NOVX coding strand, or to only a portion thereof. Nucleic acid molecules encoding fragments, homologs, derivatives and analogs of an NOVX protein of SEQ ID NOS: 2, 4, 6, 8, 10, 12, 14, 16, 18, 20, 22, 24, 26, 28, 30, or 32, or antisense nucleic acids complementary to an NOVX nucleic acid sequence of SEQ ID NOS: 1, 3, 5, 7, 9, 11, 13, 15, 17, 19, 21, 23, 25, 27, 29, and 31, are additionally provided.

In one embodiment, an antisense nucleic acid molecule is antisense to a “coding region” of the coding strand of a nucleotide sequence encoding an NOVX protein. The term “coding region” refers to the region of the nucleotide sequence comprising codons which are translated into amino acid residues. In another embodiment, the antisense nucleic acid molecule is antisense to a “noncoding region” of the coding strand of a nucleotide sequence encoding the NOVX protein. The term “noncoding region” refers to 5′ and 3′ sequences which flank the coding region that are not translated into amino acids (i.e., also referred to as 5′ and 3′ untranslated regions).

Given the coding strand sequences encoding the NOVX protein disclosed herein, antisense nucleic acids of the invention can be designed according to the rules of Watson and Crick or Hoogsteen base pairing. The antisense nucleic acid molecule can be complementary to the entire coding region of NOVX mRNA, but more preferably is an oligonucleotide that is antisense to only a portion of the coding or noncoding region of NOVX mRNA. For example, the antisense oligonucleotide can be complementary to the region surrounding the translation start site of NOVX mRNA. An antisense oligonucleotide can be, for example, about 5, 10, 15, 20, 25, 30, 35, 40, 45 or 50 nucleotides in length. An antisense nucleic acid of the invention can be constructed using chemical synthesis or enzymatic ligation reactions using procedures known in the art. For example, an antisense nucleic acid (e.g., an antisense oligonucleotide) can be chemically synthesized using naturally-occurring nucleotides or variously modified nucleotides designed to increase the biological stability of the molecules or to increase the physical stability of the duplex formed between the antisense and sense nucleic acids (e.g., phosphorothioate derivatives and acridine substituted nucleotides can be used).

Examples of modified nucleotides that can be used to generate the antisense nucleic acid include: 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding an NOVX protein to thereby inhibit expression of the protein (e.g., by inhibiting transcription and/or translation). The hybridization can be by conventional nucleotide complementarity to form a stable duplex, or, for example, in the case of an antisense nucleic acid molecule that binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface (e.g., by linking the antisense nucleic acid molecules to peptides or antibodies that bind to cell surface receptors or antigens). The antisense nucleic acid molecules can also be delivered to cells using the vectors described herein. To achieve sufficient nucleic acid molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other. See, e.g., Gaultier, et al., 1987. Nucl. Acids Res. 15: 6625-6641. The antisense nucleic acid molecule can also comprise a 2′-o-methylribonucleotide (See, e.g., Inoue, et al. 1987. Nucl. Acids Res. 15: 6131-6148) or a chimeric RNA-DNA analogue (See, e.g., Inoue, et al., 1987. FEBS Lett. 215: 327-330.

Ribozymes and PNA Moieties

Nucleic acid modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject.

In one embodiment, an antisense nucleic acid of the invention is a ribozyme. Ribozymes are catalytic RNA molecules with ribonuclease activity that are capable of cleaving a single-stranded nucleic acid, such as an mRNA, to which they have a complementary region. Thus, ribozymes (e.g., hammerhead ribozymes as described in Haselhoff and Gerlach 1988. Nature 334: 585-591) can be used to catalytically cleave NOVX mRNA transcripts to thereby inhibit translation of NOVX mRNA. A ribozyme having specificity for an NOVX-encoding nucleic acid can be designed based upon the nucleotide sequence of an NOVX cDNA disclosed herein (i.e., SEQ ID NOS: 1, 3, 5, 7, 9, 11, 13, 15, 17, 19, 21, 23, 25, 27, 29, and 31). For example, a derivative of a Tetrahymena L-19 IVS RNA can be constructed in which the nucleotide sequence of the active site is complementary to the nucleotide sequence to be cleaved in an NOVX-encoding mRNA. See, e.g., U.S. Pat. No. 4,987,071 to Cech, et al. and U.S. Pat. No. 5,116,742 to Cech, et al. NOVX mRNA can also be used to select a catalytic RNA having a specific ribonuclease activity from a pool of RNA molecules. See, e.g., Bartel et al., (1993) Science 261:1411-1418.

Alternatively, NOVX gene expression can be inhibited by targeting nucleotide sequences complementary to the regulatory region of the NOVX nucleic acid (e.g., the NOVX promoter and/or enhancers) to form triple helical structures that prevent transcription of the NOVX gene in target cells. See, e.g., Helene, 1991. Anticancer Drug Des. 6: 569-84; Helene, et al. 1992. Ann. N.Y. Acad. Sci. 660: 27-36; Maher, 1992. Bioassays 14: 807-15.

In various embodiments, the NOVX nucleic acids can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids. See, e.g., Hyrup, et al., 1996. Bioorg Med Chem 4: 5-23. As used herein, the terms “peptide nucleic acids” or “PNAs” refer to nucleic acid mimics (e.g., DNA mimics) in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup, et al., 1996. supra; Perry-O'Keefe, et al., 1996. Proc. Natl. Acad. Sci. USA 93: 14670-14675.

PNAs of NOVX can be used in therapeutic and diagnostic applications. For example, PNAs can be used as antisense or antigene agents for sequence-specific modulation of gene expression by, e.g., inducing transcription or translation arrest or inhibiting replication. PNAs of NOVX can also be used, for example, in the analysis of single base pair mutations in a gene (e.g., PNA directed PCR clamping; as artificial restriction enzymes when used in combination with other enzymes, e.g., S₁ nucleases (See, Hyrup, et al., 1996.supra); or as probes or primers for DNA sequence and hybridization (See, Hyrup, et al., 1996, supra; Perry-O'Keefe, et al., 1996. supra).

In another embodiment, PNAs of NOVX can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras of NOVX can be generated that may combine the advantageous properties of PNA and DNA. Such chimeras allow DNA recognition enzymes (e.g., RNase H and DNA polymerases) to interact with the DNA portion while the PNA portion would provide high binding affinity and specificity. PNA-DNA chimeras can be linked using linkers of appropriate lengths selected in terms of base stacking, number of bonds between the nucleobases, and orientation (see, Hyrup, et al., 1996. supra). The synthesis of PNA-DNA chimeras can be performed as described in Hyrup, et al., 1996. supra and Finn, et al., 1996. Nucl Acids Res 24: 3357-3363. For example, a DNA chain can be synthesized on a solid support using standard phosphoramidite coupling chemistry, and modified nucleoside analogs, e.g., 5′-(4-methoxytrityl)amino-5′-deoxy-thymidine phosphoramidite, can be used between the PNA and the 5′ end of DNA. See, e.g., Mag, et al., 1989. Nucl Acid Res 17: 5973-5988. PNA monomers are then coupled in a stepwise manner to produce a chimeric molecule with a 5′ PNA segment and a 3′ DNA segment. See, e.g., Finn, et al., 1996. supra. Alternatively, chimeric molecules can be synthesized with a 5′ DNA segment and a 3′ PNA segment. See, e.g., Petersen, et al., 1975. Bioorg. Med. Chem. Lett. 5: 1119-11124.

In other embodiments, the oligonucleotide may include other appended groups such as peptides (e.g., for targeting host cell receptors in vivo), or agents facilitating transport across the cell membrane (see, e.g., Letsinger, et al., 1989. Proc. Natl. Acad. Sci. U.S.A. 86: 6553-6556; Lemaitre, et al., 1987. Proc. Natl. Acad. Sci. 84: 648-652; PCT Publication No. WO88/09810) or the blood-brain barrier (see, e.g., PCT Publication No. WO 89/10134). In addition, oligonucleotides can be modified with hybridization triggered cleavage agents (see, e.g., Krol, et al., 1988. BioTechniques 6:958-976) or intercalating agents (see, e.g., Zon, 1988. Pharm. Res. 5: 539-549). To this end, the oligonucleotide may be conjugated to another molecule, e.g., a peptide, a hybridization triggered cross-linking agent, a transport agent, a hybridization-triggered cleavage agent, and the like.

NOVX Polypeptides

A polypeptide according to the invention includes a polypeptide including the amino acid sequence of NOVX polypeptides whose sequences are provided in SEQ ID NOS: 2, 4, 6, 8, 10, 12, 14, 16, 18, 20, 22, 24, 26, 28, 30, or 32. The invention also includes a mutant or variant protein any of whose residues may be changed from the corresponding residues shown in SEQ ID NOS: 2, 4, 6, 8, 10, 12, 14, 16, 18, 20, 22, 24, 26, 28, 30, or 32 while still encoding a protein that maintains its NOVX activities and physiological functions, or a functional fragment thereof.

In general, an NOVX variant that preserves NOVX-like function includes any variant in which residues at a particular position in the sequence have been substituted by other amino acids, and further include the possibility of inserting an additional residue or residues between two residues of the parent protein as well as the possibility of deleting one or more residues from the parent sequence. Any amino acid substitution, insertion, or deletion is encompassed by the invention. In favorable circumstances, the substitution is a conservative substitution as defined above.

One aspect of the invention pertains to isolated NOVX proteins, and biologically-active portions thereof, or derivatives, fragments, analogs or homologs thereof. Also provided are polypeptide fragments suitable for use as immunogens to raise anti-NOVX antibodies. In one embodiment, native NOVX proteins can be isolated from cells or tissue sources by an appropriate purification scheme using standard protein purification techniques. In another embodiment, NOVX proteins are produced by recombinant DNA techniques. Alternative to recombinant expression, an NOVX protein or polypeptide can be synthesized chemically using standard peptide synthesis techniques.

An “isolated” or “purified” polypeptide or protein or biologically-active portion thereof is substantially free of cellular material or other contaminating proteins from the cell or tissue source from which the NOVX protein is derived, or substantially free from chemical precursors or other chemicals when chemically synthesized. The language “substantially free of cellular material” includes preparations of NOVX proteins in which the protein is separated from cellular components of the cells from which it is isolated or recombinantly-produced. In one embodiment, the language “substantially free of cellular material” includes preparations of NOVX proteins having less than about 30% (by dry weight) of non-NOVX proteins (also referred to herein as a “contaminating protein”), more preferably less than about 20% of non-NOVX proteins, still more preferably less than about 10% of non-NOVX proteins, and most preferably less than about 5% of non-NOVX proteins. When the NOVX protein or biologically-active portion thereof is recombinantly-produced, it is also preferably substantially free of culture medium, i.e., culture medium represents less than about 20%, more preferably less than about 10%, and most preferably less than about 5% of the volume of the NOVX protein preparation.

The language “substantially free of chemical precursors or other chemicals” includes preparations of NOVX proteins in which the protein is separated from chemical precursors or other chemicals that are involved in the synthesis of the protein. In one embodiment, the language “substantially free of chemical precursors or other chemicals” includes preparations of NOVX proteins having less than about 30% (by dry weight) of chemical precursors or non-NOVX chemicals, more preferably less than about 20% chemical precursors or non-NOVX chemicals, still more preferably less than about 10% chemical precursors or non-NOVX chemicals, and most preferably less than about 5% chemical precursors or non-NOVX chemicals.

Biologically-active portions of NOVX proteins include peptides comprising amino acid sequences sufficiently homologous to or derived from the amino acid sequences of the NOVX proteins (e.g., the amino acid sequence shown in SEQ ID NOS: 2, 4, 6, 8, 10, 12, 14, 16, 18, 20, 22, 24, 26, 28, 30, or 32) that include fewer amino acids than the full-length NOVX proteins, and exhibit at least one activity of an NOVX protein. Typically, biologically-active portions comprise a domain or motif with at least one activity of the NOVX protein. A biologically-active portion of an NOVX protein can be a polypeptide which is, for example, 10, 25, 50, 100 or more amino acid residues in length.

Moreover, other biologically-active portions, in which other regions of the protein are deleted, can be prepared by recombinant techniques and evaluated for one or more of the functional activities of a native NOVX protein.

In an embodiment, the NOVX protein has an amino acid sequence shown SEQ ID NOS: 2, 4, 6, 8, 10, 12, 14, 16, 18, 20, 22, 24, 26, 28, 30, or 32. In other embodiments, the NOVX protein is substantially homologous to SEQ ID NOS: 2, 4, 6, 8, 10, 12, 14, 16, 18, 20, 22, 24, 26, 28, 30, or 32, and retains the functional activity of the protein of SEQ ID NOS: 2, 4, 6, 8, 10, 12, 14, 16, 18, 20, 22, 24, 26, 28, 30, or 32, yet differs in amino acid sequence due to natural allelic variation or mutagenesis, as described in detail, below. Accordingly, in another embodiment, the NOVX protein is a protein that comprises an amino acid sequence at least about 45% homologous to the amino acid sequence SEQ ID NOS: 2, 4, 6, 8, 10, 12, 14, 16, 18, 20, 22, 24, 26, 28, 30, or 32, and retains the functional activity of the NOVX proteins of SEQ ID NOS: 2, 4, 6, 8, 10, 12, 14, 16, 18, 20, 22, 24, 26, 28, 30, or 32.

Determining Homology Between Two or More Sequences

To determine the percent homology of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (i.e., as used herein amino acid or nucleic acid “homology” is equivalent to amino acid or nucleic acid “identity”).

The nucleic acid sequence homology may be determined as the degree of identity between two sequences. The homology may be determined using computer programs known in the art, such as GAP software provided in the GCG program package. See, Needleman and Wunsch, 1970. J Mol Biol 48: 443-453. Using GCG GAP software with the following settings for nucleic acid sequence comparison: GAP creation penalty of 5.0 and GAP extension penalty of 0.3, the coding region of the analogous nucleic acid sequences referred to above exhibits a degree of identity preferably of at least 70%, 75%, 80%, 85%, 90%, 95%, 98%, or 99%, with the CDS (encoding) part of the DNA sequence shown in SEQ ID NOS: 1, 3, 5, 7, 9, 11, 13, 15, 17, 19, 21, 23, 25, 27, 29,and31.

The term “sequence identity” refers to the degree to which two polynucleotide or polypeptide sequences are identical on a residue-by-residue basis over a particular region of comparison. The term “percentage of sequence identity” is calculated by comparing two optimally aligned sequences over that region of comparison, determining the number of positions at which the identical nucleic acid base (e.g., A, T, C, G, U, or I, in the case of nucleic acids) occurs in both sequences to yield the number of matched positions, dividing the number of matched positions by the total number of positions in the region of comparison (i.e., the window size), and multiplying the result by 100 to yield the percentage of sequence identity. The term “substantial identity” as used herein denotes a characteristic of a polynucleotide sequence, wherein the polynucleotide comprises a sequence that has at least 80 percent sequence identity, preferably at least 85 percent identity and often 90 to 95 percent sequence identity, more usually at least 99 percent sequence identity as compared to a reference sequence over a comparison region.

Chimeric and Fusion Proteins

The invention also provides NOVX chimeric or fusion proteins. As used herein, an NOVX “chimeric protein” or “fusion protein” comprises an NOVX polypeptide operatively-linked to a non-NOVX polypeptide. An “NOVX polypeptide” refers to a polypeptide having an amino acid sequence corresponding to an NOVX protein SEQ ID NOS: 2, 4, 6, 8, 10, 12, 14, 16, 18, 20, 22, 24, 26, 28, 30, or 32, whereas a “non-NOVX polypeptide” refers to a polypeptide having an amino acid sequence corresponding to a protein that is not substantially homologous to the NOVX protein, e.g., a protein that is different from the NOVX protein and that is derived from the same or a different organism. Within an NOVX fusion protein the NOVX polypeptide can correspond to all or a portion of an NOVX protein. In one embodiment, an NOVX fusion protein comprises at least one biologically-active portion of an NOVX protein. In another embodiment, an NOVX fusion protein comprises at least two biologically-active portions of an NOVX protein. In yet another embodiment, an NOVX fusion protein comprises at least three biologically-active portions of an NOVX protein. Within the fusion protein, the term “operatively-linked” is intended to indicate that the NOVX polypeptide and the non-NOVX polypeptide are fused in-frame with one another. The non-NOVX polypeptide can be fused to the N-terminus or C-terminus of the NOVX polypeptide.

In one embodiment, the fusion protein is a GST-NOVX fusion protein in which the NOVX sequences are fused to the C-terminus of the GST (glutathione S-transferase) sequences. Such fusion proteins can facilitate the purification of recombinant NOVX polypeptides.

In another embodiment, the fusion protein is an NOVX protein containing a heterologous signal sequence at its N-terminus. In certain host cells (e.g., mammalian host cells), expression and/or secretion of NOVX can be increased through use of a heterologous signal sequence.

In yet another embodiment, the fusion protein is an NOVX-immunoglobulin fusion protein in which the NOVX sequences are fused to sequences derived from a member of the immunoglobulin protein family. The NOVX-immunoglobulin fusion proteins of the invention can be incorporated into pharmaceutical compositions and administered to a subject to inhibit an interaction between an NOVX ligand and an NOVX protein on the surface of a cell, to thereby suppress NOVX-mediated signal transduction in vivo. The NOVX-immunoglobulin fusion proteins can be used to affect the bioavailability of an NOVX cognate ligand. Inhibition of the NOVX ligand/NOVX interaction may be useful therapeutically for both the treatment of proliferative and differentiative disorders, as well as modulating (e.g. promoting or inhibiting) cell survival. Moreover, the NOVX-immunoglobulin fusion proteins of the invention can be used as immunogens to produce anti-NOVX antibodies in a subject, to purify NOVX ligands, and in screening assays to identify molecules that inhibit the interaction of NOVX with an NOVX ligand.

An NOVX chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, e.g., Ausubel, et al. (eds.) CURRENT PROTOCOLS IN MOLECULAR BIOLOGY, John Wiley & Sons, 1992). Moreover, many expression vectors are commercially available that already encode a fusion moiety (e.g., a GST polypeptide). An NOVX-encoding nucleic acid can be cloned into such an expression vector such that the fusion moiety is linked in-frame to the NOVX protein.

NOVX Agonists and Antagonists

The invention also pertains to variants of the NOVX proteins that function as either NOVX agonists (i.e., mimetics) or as NOVX antagonists. Variants of the NOVX protein can be generated by mutagenesis (e.g., discrete point mutation or truncation of the NOVX protein). An agonist of the NOVX protein can retain substantially the same, or a subset of, the biological activities of the naturally occurring form of the NOVX protein. An antagonist of the NOVX protein can inhibit one or more of the activities of the naturally occurring form of the NOVX protein by, for example, competitively binding to a downstream or upstream member of a cellular signaling cascade which includes the NOVX protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the NOVX proteins.

Variants of the NOVX proteins that function as either NOVX agonists (i.e., mimetics) or as NOVX antagonists can be identified by screening combinatorial libraries of mutants (e.g., truncation mutants) of the NOVX proteins for NOVX protein agonist or antagonist activity. In one embodiment, a variegated library of NOVX variants is generated by combinatorial mutagenesis at the nucleic acid level and is encoded by a variegated gene library. A variegated library of NOVX variants can be produced by, for example, enzymatically ligating a mixture of synthetic oligonucleotides into gene sequences such that a degenerate set of potential NOVX sequences is expressible as individual polypeptides, or alternatively, as a set of larger fusion proteins (e.g., for phage display) containing the set of NOVX sequences therein. There are a variety of methods which can be used to produce libraries of potential NOVX variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential NOVX sequences. Methods for synthesizing degenerate oligonucleotides are well-known within the art. See, e.g., Narang, 1983. Tetrahedron 39: 3; Itakura, et al., 1984. Annu. Rev. Biochem. 53: 323; Itakura, et al., 1984. Science 198: 1056; Ike, et al., 1983. Nucl. Acids Res. 11: 477.

Polypeptide Libraries

In addition, libraries of fragments of the NOVX protein coding sequences can be used to generate a variegated population of NOVX fragments for screening and subsequent selection of variants of an NOVX protein. In one embodiment, a library of coding sequence fragments can be generated by treating a double stranded PCR fragment of an NOVX coding sequence with a nuclease under conditions wherein nicking occurs only about once per molecule, denaturing the double stranded DNA, renaturing the DNA to form double-stranded DNA that can include sense/antisense pairs from different nicked products, removing single stranded portions from reformed duplexes by treatment with SI nuclease, and ligating the resulting fragment library into an expression vector. By this method, expression libraries can be derived which encodes N-terminal and internal fragments of various sizes of the NOVX proteins.

Various techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of NOVX proteins. The most widely used techniques, which are amenable to high throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique that enhances the frequency of functional mutants in the libraries, can be used in combination with the screening assays to identify NOVX variants. See, e.g., Arkin and Yourvan, 1992. Proc. Natl. Acad. Sci. USA 89: 7811-7815; Delgrave, et al., 1993. Protein Engineering 6:327-331.

Anti-NOVX Antibodies

Also included in the invention are antibodies to NOVX proteins, or fragments of NOVX proteins. The term “antibody” as used herein refers to immunoglobulin molecules and immunologically active portions of immunoglobulin (Ig) molecules, i.e., molecules that contain an antigen binding site that specifically binds (immunoreacts with) an antigen. Such antibodies include, but are not limited to, polyclonal, monoclonal, chimeric, single chain, F_{ab}, F_{ab′} and F(_{ab′})₂ fragments, and an F_{ab} expression library. In general, an antibody molecule obtained from humans relates to any of the classes IgG, IgM, IgA, IgE and IgD, which differ from one another by the nature of the heavy chain present in the molecule. Certain classes have subclasses as well, such as IgG₁, IgG₂, and others. Furthermore, in humans, the light chain may be a kappa chain or a lambda chain. Reference herein to antibodies includes a reference to all such classes, subclasses and types of human antibody species.

An isolated NOVX-related protein of the invention may be intended to serve as an antigen, or a portion or fragment thereof, and additionally can be used as an immunogen to generate antibodies that immunospecifically bind the antigen, using standard techniques for polyclonal and monoclonal antibody preparation. The full-length protein can be used or, alternatively, the invention provides antigenic peptide fragments of the antigen for use as immunogens. An antigenic peptide fragment comprises at least 6 amino acid residues of the amino acid sequence of the full length protein and encompasses an epitope thereof such that an antibody raised against the peptide forms a specific immune complex with the full length protein or with any fragment that contains the epitope. Preferably, the antigenic peptide comprises at least 10 amino acid residues, or at least 15 amino acid residues, or at least 20 amino acid residues, or at least 30 amino acid residues. Preferred epitopes encompassed by the antigenic peptide are regions of the protein that are located on its surface; commonly these are hydrophilic regions.

In certain embodiments of the invention, at least one epitope encompassed by the antigenic peptide is a region of NOVX-related protein that is located on the surface of the protein, e.g., a hydrophilic region. A hydrophobicity analysis of the human NOVX-related protein sequence will indicate which regions of a NOVX-related protein are particularly hydrophilic and, therefore, are likely to encode surface residues useful for targeting antibody production. As a means for targeting antibody production, hydropathy plots showing regions of hydrophilicity and hydrophobicity may be generated by any method well known in the art, including, for example, the Kyte Doolittle or the Hopp Woods methods, either with or without Fourier transformation. See, e.g., Hopp and Woods, 1981, Proc. Nat. Acad. Sci. USA 78: 3824-3828; Kyte and Doolittle 1982, J. Mol. Biol. 157: 105-142, each of which is incorporated herein by reference in its entirety. Antibodies that are specific for one or more domains within an antigenic protein, or derivatives, fragments, analogs or homologs thereof, are also provided herein.

A protein of the invention, or a derivative, fragment, analog, homolog or ortholog thereof, may be utilized as an immunogen in the generation of antibodies that immunospecifically bind these protein components.

Various procedures known within the art may be used for the production of polyclonal or monoclonal antibodies directed against a protein of the invention, or against derivatives, fragments, analogs homologs or orthologs thereof (see, for example, Antibodies: A Laboratory Manual, Harlow and Lane, 1988, Cold Spring Harbor Laboratory Press, Cold Spring Harbor, NY, incorporated herein by reference). Some of these antibodies are discussed below.

Polyclonal Antibodies

For the production of polyclonal antibodies, various suitable host animals (e.g., rabbit, goat, mouse or other mammal) may be immunized by one or more injections with the native protein, a synthetic variant thereof, or a derivative of the foregoing. An appropriate immunogenic preparation can contain, for example, the naturally occurring immunogenic protein, a chemically synthesized polypeptide representing the immunogenic protein, or a recombinantly expressed immunogenic protein. Furthermore, the protein may be conjugated to a second protein known to be immunogenic in the mammal being immunized. Examples of such immunogenic proteins include but are not limited to keyhole limpet hemocyanin, serum albumin, bovine thyroglobulin, and soybean trypsin inhibitor. The preparation can further include an adjuvant. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.), adjuvants usable in humans such as Bacille Calmette-Guerin and Corynebacterium parvum, or similar immunostimulatory agents. Additional examples of adjuvants which can be employed include MPL-TDM adjuvant (monophosphorpl Lipid A, synthetic trehalose dicorynomycolate).

The polyclonal antibody molecules directed against the immunogenic protein can be isolated from the mammal (e.g., from the blood) and further purified by well known techniques, such as affinity chromatography using protein A or protein G, which provide primarily the IgG fraction of immune serum. Subsequently, or alternatively, the specific antigen which is the target of the immunoglobulin sought, or an epitope thereof, may be immobilized on a column to purify the immune specific antibody by immunoaffinity chromatography. Purification of immunoglobulins is discussed, for example, by D. Wilkinson (The Scientist, published by The Scientist, Inc., Philadelphia Pa., Vol. 14, No. 8 (Apr. 17, 2000), pp. 25-28).

Monoclonal Antibodies

The term “monoclonal antibody” (MAb) or “monoclonal antibody composition”, as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs thus contain an antigen binding site capable of immunoreacting with a particular epitope of the antigen characterized by a unique binding affinity for it.

Monoclonal antibodies can be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes can be immunized in vitro.

The immunizing agent will typically include the protein antigen, a fragment thereof or a fusion protein thereof. Generally, either peripheral blood lymphocytes are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, MONOCLONAL ANTIBODIES: PRINCIPLES AND PRACTICE, Academic Press, (1986) pp. 59-103). Immortalized cell lines are usually transformed mammalian cells, particularly myeloma cells of rodent, bovine and human origin. Usually, rat or mouse myeloma cell lines are employed. The hybridoma cells can be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies (Kozbor, J. Immunol., 133:3001 (1984); Brodeur et al., MONOCLONAL ANTIBODY PRODUCTION TECHNIQUES AND APPLICATIONS, Marcel Dekker, Inc., New York, (1987) pp. 51-63).

The culture medium in which the hybridoma cells are cultured can then be assayed for the presence of monoclonal antibodies directed against the antigen. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980). Preferably, antibodies having a high degree of specificity and a high binding affinity for the target antigen are isolated.

After the desired hybridoma cells are identified, the clones can be subcloned by limiting dilution procedures and grown by standard methods. Suitable culture media for this purpose include, for example, Dulbecco's Modified Eagle's Medium and RPMI-1640 medium. Alternatively, the hybridoma cells can be grown in vivo as ascites in a mammal.

The monoclonal antibodies secreted by the subclones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies can also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA can be placed into expression vectors; which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences (U.S. Pat. No. 4,816,567; Morrison, Nature 368, 812-13 (1994)) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

Humanized Antibodies

The antibodies directed against the protein antigens of the invention can further comprise humanized antibodies or human antibodies. These antibodies are suitable for administration to humans without engendering an immune response by the human against the administered immunoglobulin. Humanized forms of antibodies are chimeric immunoglobulins, immunoglobulin chains or fragments thereof (such as Fv, Fab, Fab′, F(ab′)₂ or other antigen-binding subsequences of antibodies) that are principally comprised of the sequence of a human immunoglobulin, and contain minimal sequence derived from a non-human immunoglobulin. Humanization can be performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. (See also U.S. Pat. No. 5,225,539.) In some instances, Fv framework residues of the human immunoglobulin are replaced by corresponding non-human residues. Humanized antibodies can also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the framework regions are those of a human immunoglobulin consensus sequence. The humanized antibody optimally also will comprise at least a portion of an immunoglobulin constant region (Fc), typically that of a human immunoglobulin (Jones et al., 1986; Riechmann et al., 1988; and Presta, Curr. Op. Struct. Biol., 2:593-596 (1992)).

Human Antibodies

Fully human antibodies relate to antibody molecules in which essentially the entire sequences of both the light chain and the heavy chain, including the CDRs, arise from human genes. Such antibodies are termed “human antibodies”, or “fully human antibodies” herein. Human monoclonal antibodies can be prepared by the trioma technique; the human B-cell hybridoma technique (see Kozbor, et al., 1983 Immunol Today 4: 72) and the EBV hybridoma technique to produce human monoclonal antibodies (see Cole, et al., 1985 In: MONOCLONAL ANTIBODIES AND CANCER THERAPY, Alan R. Liss, Inc., pp. 77-96). Human monoclonal antibodies may be utilized in the practice of the present invention and may be produced by using human hybridomas (see Cote, et al., 1983. Proc Natl Acad Sci USA 80: 2026-2030) or by transforming human B-cells with Epstein Barr Virus in vitro (see Cole, et al., 1985 In: MONOCLONAL ANTIBODIES AND CANCER THERAPY, Alan R. Liss, Inc., pp. 77-96).

In addition, human antibodies can also be produced using additional techniques, including phage display libraries (Hoogenboom and Winter, J. Mol. Biol., 227:381 (1991); Marks et al., J. Mol. Biol., 222:581 (1991)). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in Marks et al. (Bio/Technology 10, 779-783 (1992)); Lonberg et al. (Nature 368 856-859 (1994)); Morrison (Nature 368, 812-13 (1994)); Fishwild et al,( Nature Biotechnology 14, 845-51 (1996)); Neuberger (Nature Biotechnology 14, 826 (1996)); and Lonberg and Huszar (Intern. Rev. Immunol. 13 65-93 (1995)).

Human antibodies may additionally be produced using transgenic nonhuman animals which are modified so as to produce fully human antibodies rather than the animal's endogenous antibodies in response to challenge by an antigen. (See PCT publication WO94/02602). The endogenous genes encoding the heavy and light immunoglobulin chains in the nonhuman host have been incapacitated, and active loci encoding human heavy and light chain immunoglobulins are inserted into the host's genome. The human genes are incorporated, for example, using yeast artificial chromosomes containing the requisite human DNA segments. An animal which provides all the desired modifications is then obtained as progeny by crossbreeding intermediate transgenic animals containing fewer than the full complement of the modifications. The preferred embodiment of such a nonhuman animal is a mouse, and is termed the Xenomouse™ as disclosed in PCT publications WO 96/33735 and WO 96/34096. This animal produces B cells which secrete fully human immunoglobulins. The antibodies can be obtained directly from the animal after immunization with an immunogen of interest, as, for example, a preparation of a polyclonal antibody, or alternatively from immortalized B cells derived from the animal, such as hybridomas producing monoclonal antibodies. Additionally, the genes encoding the immunoglobulins with human variable regions can be recovered and expressed to obtain the antibodies directly, or can be further modified to obtain analogs of antibodies such as, for example, single chain Fv molecules.

An example of a method of producing a nonhuman host, exemplified as a mouse, lacking expression of an endogenous immunoglobulin heavy chain is disclosed in U.S. Pat. No. 5,939,598. It can be obtained by a method including deleting the J segment genes from at least one endogenous heavy chain locus in an embryonic stem cell to prevent rearrangement of the locus and to prevent formation of a transcript of a rearranged immunoglobulin heavy chain locus, the deletion being effected by a targeting vector containing a gene encoding a selectable marker; and producing from the embryonic stem cell a transgenic mouse whose somatic and germ cells contain the gene encoding the selectable marker.

A method for producing an antibody of interest, such as a human antibody, is disclosed in U.S. Pat. No. 5,916,771. It includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain.

In a further improvement on this procedure, a method for identifying a clinically relevant epitope on an immunogen, and a correlative method for selecting an antibody that binds immunospecifically to the relevant epitope with high affinity, are disclosed in PCT publication WO 99/53049.

F_{ab} Fragments and Single Chain Antibodies

According to the invention, techniques can be adapted for the production of single-chain antibodies specific to an antigenic protein of the invention (see e.g., U.S. Pat. No. 4,946,778). In addition, methods can be adapted for the construction of F_{ab} expression libraries (see e.g., Huse, et al., 1989 Science 246: 1275-1281) to allow rapid and effective. identification of monoclonal F_{ab} fragments with the desired specificity for a protein or derivatives, fragments, analogs or homologs thereof. Antibody fragments that contain the idiotypes to a protein antigen may be produced by techniques known in the art including, but not limited to: (i) an F(_{ab′)2} fragment produced by pepsin digestion of an antibody molecule; (ii) an F_{ab} fragment generated by reducing the disulfide bridges of an F(_{ab′)2} fragment; (iii) an F_{ab} fragment generated by the treatment of the antibody molecule with papain and a reducing agent and (iv) Fᵥ fragments.

Bispecific Antibodies

Bispecific antibodies are monoclonal, preferably human or humanized, antibodies that have binding specificities for at least two different antigens. In the present case, one of the binding specificities is for an antigenic protein of the invention. The second binding target is any other antigen, and advantageously is a cell-surface protein or receptor or receptor subunit.

Methods for making bispecific antibodies are known in the art. Traditionally, the recombinant production of bispecific antibodies is based on the co-expression of two immunoglobulin heavy-chain/light-chain pairs, where the two heavy chains have different specificities (Milstein and Cuello, Nature, 305:537-539 (1983)). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published May 13, 1993, and in Traunecker et al., 1991 EMBO J., 10:3655-3659.

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies see, for example, Suresh et al., Methods in Enzymology, 121:210 (1986).

According to another approach described in WO 96/27011, the interface between a pair of antibody molecules can be engineered to maximize the percentage of heterodimers which are recovered from recombinant cell culture. The preferred interface comprises at least a part of the CH3 region of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies can be prepared as full length antibodies or antibody fragments (e.g. F(ab′)₂ bispecific antibodies). Techniques for generating bispecific antibodies from antibody fragments have been described in the literature. For example, bispecific antibodies can be prepared using chemical linkage. Brennan et al., Science 229:81,(1985) describe a procedure wherein intact antibodies are proteolytically cleaved to generate F(ab′)₂ fragments.

These fragments are reduced in the presence of the dithiol com26S protease regulatory subunit 4 g agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Additionally, Fab′ fragments can be directly recovered from E. coli and chemically coupled to form bispecific antibodies. Shalaby et al., J. Exp. Med. 175:217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described. For example, bispecific antibodies have been produced using leucine zippers. Kostelny et al., J. Immunol. 148(5): 1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA 90:6444-6448 (1993) has provided an alternative mechanism for making bispecific antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See, Gruber et al., J. Immunol. 152:5368 (1994).

Antibodies with more than two valencies are contemplated. For example, trispecific antibodies can be prepared. Tutt et al., J. Immunol. 147:60 (1991).

Exemplary bispecific antibodies can bind to two different epitopes, at least one of which originates in the protein antigen of the invention. Alternatively, an anti-antigenic arm of an immunoglobulin molecule can be combined with an arm which binds to a triggering molecule on a leukocyte such as a T-cell receptor molecule (e.g. CD2, CD3, CD28, or B7), or Fc receptors for IgG (FcγR), such as FcγRI (CD64), FcγRII (CD32) and FcγRI (CD16) so as to focus cellular defense mechanisms to the cell expressing the particular antigen. Bispecific antibodies can also be used to direct cytotoxic agents to cells which express a particular antigen. These antibodies possess an antigen-binding arm and an arm which binds a cytotoxic agent or a radionuclide chelator, such as EOTUBE, DPTA, DOTA, or TETA. Another bispecific antibody of interest binds the protein antigen described herein and further binds tissue factor (TF).

Heteroconjugate Antibodies

Heteroconjugate antibodies are also within the scope of the present invention. Heteroconjugate antibodies are composed of two covalently joined antibodies. Such antibodies have, for example, been proposed to target immune system cells to unwanted cells (U.S. Pat. No. 4,676,980), and for treatment of HIV infection (WO 91/00360; WO 92/200373; EP 03089). It is contemplated that the antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

Effector Function Engineering

It can be desirable to modify the antibody of the invention with respect to effector function, so as to enhance, e.g., the effectiveness of the antibody in treating cancer. For example, cysteine residue(s) can be introduced into the Fc region, thereby allowing interchain disulfide bond formation in this region. The homodimeric antibody thus generated can have improved internalization capability and/or increased complement-mediated cell killing and antibody-dependent cellular cytotoxicity (ADCC). See Caron et al., J. Exp Med., 176: 1191-1195 (1992) and Shopes, J. Immunol., 148: 2918-2922 (1992). Homodimeric antibodies with enhanced anti-tumor activity can also be prepared using heterobifunctional cross-linkers as described in Wolff et al. Cancer Research, 53: 2560-2565 (1993). Alternatively, an antibody can be engineered that has dual Fc regions and can thereby have enhanced complement lysis and ADCC capabilities. See Stevenson et al., Anti-Cancer Drug Design, 3: 219-230 (1989).

Immunoconjugates

The invention also pertains to immunoconjugates comprising an antibody conjugated to a cytotoxic agent such as a chemotherapeutic agent, toxin (e.g., an enzymatically active toxin of bacterial, fungal, plant, or animal origin, or fragments thereof), or a radioactive isotope (i.e., a radioconjugate).

Chemotherapeutic agents useful in the generation of such immunoconjugates have been described above. Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes. A variety of radionuclides are available for the production of radioconjugated antibodies. Examples include ²¹²Bi, ¹³¹I, ¹³¹In, ⁹⁰Y, and ¹⁸⁶Re.

Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science, 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026.

In another embodiment, the antibody can be conjugated to a “receptor” (such streptavidin) for utilization in tumor pretargeting wherein the antibody-receptor conjugate is administered to the patient, followed by removal of unbound conjugate from the circulation using a clearing agent and then administration of a “ligand” (e.g., avidin) that is in turn conjugated to a cytotoxic agent.

In one embodiment, methods for the screening of antibodies that possess the desired specificity include, but are not limited to, enzyme-linked immunosorbent assay (ELISA) and other immunologically-mediated techniques known within the art. In a specific embodiment, selection of antibodies that are specific to a particular domain of an NOVX protein is facilitated by generation of hybridomas that bind to the fragment of an NOVX protein possessing such a domain. Thus, antibodies that are specific for a desired domain within an NOVX protein, or derivatives, fragments, analogs or homologs thereof, are also provided herein.

Anti-NOVX antibodies may be used in methods known within the art relating to the localization and/or quantitation of an NOVX protein (e.g., for use in measuring levels of the NOVX protein within appropriate physiological samples, for use in diagnostic methods, for use in imaging the protein, and the like). In a given embodiment, antibodies for NOVX proteins, or derivatives, fragments, analogs or homologs thereof, that contain the antibody derived binding domain, are utilized as pharmacologically-active compounds (hereinafter “Therapeutics”).

An anti-NOVX antibody (e.g., monoclonal antibody) can be used to isolate an NOVX polypeptide by standard techniques, such as affinity chromatography or immunoprecipitation. An anti-NOVX antibody can facilitate the purification of natural NOVX polypeptide from cells and of recombinantly-produced NOVX polypeptide expressed in host cells. Moreover, an anti-NOVX antibody can be used to detect NOVX protein (e.g., in a cellular lysate or cell supernatant) in order to evaluate the abundance and pattern of expression of the NOVX protein. Anti-NOVX antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

NOVX Recombinant Expression Vectors and Host Cells

Another aspect of the invention pertains to vectors, preferably expression vectors, containing a nucleic acid encoding an NOVX protein, or derivatives, fragments, analogs or homologs thereof. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively-linked to the nucleic acid sequence to be expressed. Within a recombinant expression vector, “operably-linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell).

The term “regulatory sequence” is intended to includes promoters, enhancers and other expression control elements (e.g., polyadenylation signals). Such regulatory sequences are described, for example, in Goeddel, GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., NOVX proteins, mutant forms of NOVX proteins, fusion proteins, etc.).

The recombinant expression vectors of the invention can be designed for expression of NOVX proteins in prokaryotic or eukaryotic cells. For example, NOVX proteins can be expressed in bacterial cells such as Escherichia coli, insect cells (using baculovirus expression vectors) yeast cells or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein.

Examples of suitable inducible non-fusion E. coli expression vectors include pTrc (Amrann et al., (1988) Gene 69:301-315) and pET 11d (Studier et al., GENE EXPRESSION TECHNOLOGY: METHODS IN ENZYMOLOGY 185, Academic Press, San Diego, Calif. (1990) 60-89).

One strategy to maximize recombinant protein expression in E. coli is to express the protein in a host bacteria with an impaired capacity to proteolytically cleave the recombinant protein. See, e.g., Gottesman, GENE EXPRESSION TECHNOLOGY: METHODS IN ENZYMOLOGY 185, Academic Press, San Diego, Calif. (1990) 119-128. Another strategy is to alter the nucleic acid sequence of the nucleic acid to be inserted into an expression vector so that the individual codons for each amino acid are those preferentially utilized in E. coli (see, e.g., Wada, et al., 1992. Nucl. Acids Res. 20: 2111-2118). Such alteration of nucleic acid sequences of the invention can be carried out by standard DNA synthesis techniques.

In another embodiment, the NOVX expression vector is a yeast expression vector. Examples of vectors for expression in yeast Saccharomyces cerivisae include pYepSec1 (Baldari, et al., 1987. EMBO J. 6: 229-234), pMFa (Kurjan and Herskowitz, 1982. Cell 30: 933-943), pJRY88 (Schultz et al., 1987. Gene 54: 113-123), pYES2 (Invitrogen Corporation, San Diego, Calif.), and picZ (In Vitrogen Corp, San Diego, Calif.).

Alternatively, NOVX can be expressed in insect cells using baculovirus expression vectors. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39).

In yet another embodiment, a nucleic acid of the invention is expressed in mammalian cells using a mammalian expression vector. Examples of mammalian expression vectors include pCDM8 (Seed, 1987. Nature 329: 840) and pMT2PC (Kaufman, et al., 1987. EMBO J. 6: 187-195). When used in mammalian cells, the expression vector's control functions are often provided by viral regulatory elements. For example, commonly used promoters are derived from polyoma, adenovirus 2, cytomegalovirus, and simian virus 40. For other suitable expression systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Banerji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the α-fetoprotein promoter (Campes and Tilghman, 1989. Genes Dev. 3: 537-546).

The invention further provides a recombinant expression vector comprising a DNA molecule of the invention cloned into the expression vector in an antisense orientation. That is, the DNA molecule is operatively-linked to a regulatory sequence in a manner that allows for expression (by transcription of the DNA molecule) of an RNA molecule that is antisense to NOVX mRNA. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see, e.g., Weintraub, et al., “Antisense RNA as a molecular tool for genetic analysis,” Reviews-Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, NOVX protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (MOLECULAR CLONING: A LABORATORY MANUAL. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding NOVX or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) NOVX protein. Accordingly, the invention further provides methods for producing NOVX protein using the host cells of the invention. In one embodiment, the method comprises culturing the host cell of invention (into which a recombinant expression vector encoding NOVX protein has been introduced) in a suitable medium such that NOVX protein is produced. In another embodiment, the method further comprises isolating NOVX protein from the medium or the host cell.

Transgenic NOVX Animals

The host cells of the invention can also be used to produce non-human transgenic animals. For example, in one embodiment, a host cell of the invention is a fertilized oocyte or an embryonic stem cell into which NOVX protein-coding sequences have been introduced. Such host cells can then be used to create non-human transgenic animals in which exogenous NOVX sequences have been introduced into their genome or homologous recombinant animals in which endogenous NOVX sequences have been altered. Such animals are useful for studying the function and/or activity of NOVX protein and for identifying and/or evaluating modulators of NOVX protein activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA that is integrated into the genome of a cell from which a transgenic animal develops and that remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous NOVX gene has been altered by homologous recombination between the endogenous gene and an exogenous DNA molecule introduced into a cell of the animal, e.g., an embryonic cell of the animal, prior to development of the animal.

A transgenic animal of the invention can be created by introducing NOVX-encoding nucleic acid into the male pronuclei of a fertilized oocyte (e.g., by microinjection, retroviral infection) and allowing the oocyte to develop in a pseudopregnant female foster animal. The human NOVX cDNA sequences SEQ ID NOS: 1, 3, 5, 7, 9, 11, 13, 15, 17, 19, 21, 23, 25, 27, 29, and 31 can be introduced as a transgene into the genome of a non-human animal. Alternatively, a non-human homologue of the human NOVX gene, such as a mouse NOVX gene, can be isolated based on hybridization to the human NOVX cDNA (described further supra) and used as a transgene. Intronic sequences and polyadenylation signals can also be included in the transgene to increase the efficiency of expression of the transgene. A tissue-specific regulatory sequence(s) can be operably-linked to the NOVX transgene to direct expression of NOVX protein to particular cells. Methods for generating transgenic animals via embryo manipulation and microinjection, particularly animals such as mice, have become conventional in the art and are described, for example, in U.S. Pat. Nos. 4,736,866; 4,870,009; and 4,873,191; and Hogan, 1986. In: MANIPULATING THE MOUSE EMBRYO, Cold Spring Harbor Laboratory Press, Cold Spring Harbor, N.Y. Similar methods are used for production of other transgenic animals. A transgenic founder animal can be identified based upon the presence of the NOVX transgene in its genome and/or expression of NOVX mRNA in tissues or cells of the animals. A transgenic founder animal can then be used to breed additional animals carrying the transgene. Moreover, transgenic animals carrying a transgene-encoding NOVX protein can further be bred to other transgenic animals carrying other transgenes.

To create a homologous recombinant animal, a vector is prepared which contains at least a portion of an NOVX gene into which a deletion, addition or substitution has been introduced to thereby alter, e.g., functionally disrupt, the NOVX gene. The NOVX gene can be a human gene (e.g., the cDNA of SEQ ID NOS: 1, 3, 5, 7, 9, 11, 13, 15, 17, 19, 21, 23, 25, 27, 29, and 31), but more preferably, is a non-human homologue of a human NOVX gene. For example, a mouse homologue of human NOVX gene of SEQ ID NOS: 1, 3, 5, 7, 9, 11, 13, 15, 17, 19, 21, 23, 25, 27, 29, and 31 can be used to construct a homologous recombination vector suitable for altering an endogenous NOVX gene in the mouse genome. In one embodiment, the vector is designed such that, upon homologous recombination, the endogenous NOVX gene is functionally disrupted (i.e., no longer encodes a functional protein; also referred to as a “knock out” vector).

Alternatively, the vector can be designed such that, upon homologous recombination, the endogenous NOVX gene is mutated or otherwise altered but still encodes functional protein (e.g., the upstream regulatory region can be altered to thereby alter the expression of the endogenous NOVX protein). In the homologous recombination vector, the altered portion of the NOVX gene is flanked at its 5′-and 3′-termini by additional nucleic acid of the NOVX gene to allow for homologous recombination to occur between the exogenous NOVX gene carried by the vector and an endogenous NOVX gene in an embryonic stem cell. The additional flanking NOVX nucleic acid is of sufficient length for successful homologous recombination with the endogenous gene. Typically, several kilobases of flanking DNA (both at the 5′-and 3′-termini) are included in the vector. See, e.g., Thomas, et al., 1987. Cell 51: 503 for a description of homologous recombination vectors. The vector is ten introduced into an embryonic stem cell line (e.g., by electroporation) and cells in which the introduced NOVX gene has homologously-recombined with the endogenous NOVX gene are selected. See, e.g., Li, et al., 1992. Cell 69: 915.

The selected cells are then injected into a blastocyst of an animal (e.g., a mouse) to form aggregation chimeras. See, e.g., Bradley, 1987. In: TERATOCARCINOMAS AND EMBRYONIC STEM CELLS: A PRACTICAL APPROACH, Robertson, ed. IRL, Oxford, pp. 113-152. A chimeric embryo can then be implanted into a suitable pseudopregnant female foster animal and the embryo brought to term. Progeny harboring the homologously-recombined DNA in their germ cells can be used to breed animals in which all cells of the animal contain the homologously-recombined DNA by germline transmission of the transgene. Methods for constructing homologous recombination vectors and homologous recombinant animals are described further in Bradley, 1991. Curr. Opin. Biotechnol. 2: 823-829; PCT International Publication Nos.: WO 90/11354; WO 91/01140; WO 92/0968; and WO 93/04169.

In another embodiment, transgenic non-humans animals can be produced that contain selected systems that allow for regulated expression of the transgene. One example of such a system is the cre/oxP recombinase system of bacteriophage P1. For a description of the cre/loxP recombinase system, See, e.g., Lakso, et al., 1992. Proc. Natl. Acad. Sci. USA 89: 6232-6236. Another example of a recombinase system is the FLP recombinase system of Saccharomyces cerevisiae. See, O'Gorman, et al., 1991. Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, et al., 1997. Nature 385: 810-813. In brief, a cell (e.g., a somatic cell) from the transgenic animal can be isolated and induced to exit the growth cycle and enter G₀ phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell (e.g., the somatic cell) is isolated.

Pharmaceutical Compositions

The NOVX nucleic acid molecules, NOVX proteins, and anti-NOVX antibodies (also referred to herein as “active compounds”) of the invention, and derivatives, fragments, analogs and homologs thereof, can be incorporated into pharmaceutical compositions suitable for administration. Such compositions typically comprise the nucleic acid molecule, protein, or antibody and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., an NOVX protein or anti-NOVX antibody) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (see, e.g., U.S. Pat. No. 5,328,470) or by stereotactic injection (see, e.g., Chen, et al., 1994. Proc. Natl. Acad. Sci. USA 91: 3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

The pharmaceutical compositions can be included in a container, pack, or dispenser together with instructions for administration.

Screening and Detection Methods

The isolated nucleic acid molecules of the invention can be used to express NOVX protein (e.g., via a recombinant expression vector in a host cell in gene therapy applications), to detect NOVX mRNA (e.g., in a biological sample) or a genetic lesion in an NOVX gene, and to modulate NOVX activity, as described further, below. In addition, the NOVX proteins can be used to screen drugs or compounds that modulate the NOVX protein activity or expression as well as to treat disorders characterized by insufficient or excessive production of NOVX protein or production of NOVX protein forms that have decreased or aberrant activity compared to NOVX wild-type protein (e.g.; diabetes (regulates insulin release); obesity (binds and transport lipids); metabolic disturbances associated with obesity, the metabolic syndrome X as well as anorexia and wasting disorders associated with chronic diseases and various cancers, and infectious disease(possesses anti-microbial activity) and the various dyslipidemias. In addition, the anti-NOVX antibodies of the invention can be used to detect and isolate NOVX proteins and modulate NOVX activity. In yet a further aspect, the invention can be used in methods to influence appetite, absorption of nutrients and the disposition of metabolic substrates in both a positive and negative fashion.

The invention further pertains to novel agents identified by the screening assays described herein and uses thereof for treatments as described, supra.

Screening Assays The invention provides a method (also referred to herein as a “screening assay”) for identifying modulators, i.e., candidate or test compounds or agents (e.g., peptides, peptidomimetics, small molecules or other drugs) that bind to NOVX proteins or have a stimulatory or inhibitory effect on, e.g., NOVX protein expression or NOVX protein activity. The invention also includes compounds identified in the screening assays described herein.

In one embodiment, the invention provides assays for screening candidate or test compounds which bind to or modulate the activity of the membrane-bound form of an NOVX protein or polypeptide or biologically-active portion thereof. The test compounds of the invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds. See, e.g., Lam, 1997. Anticancer Drug Design 12: 145.

A “small molecule” as used herein, is meant to refer to a composition that has a molecular weight of less than about 5 kD and most preferably less than about 4 kD. Small molecules can be, e.g., nucleic acids, peptides, polypeptides, peptidomimetics, carbohydrates, lipids or other organic or inorganic molecules. Libraries of chemical and/or biological mixtures, such as fungal, bacterial, or algal extracts, are known in the art and can be screened with any of the assays of the invention.

Examples of methods for the synthesis of molecular libraries can be found in the art, for example in: DeWitt, et al., 1993. Proc. Natl. Acad. Sci. U.S.A. 90: 6909; Erb, et al., 1994. Proc. Natl. Acad. Sci. U.S.A. 91: 11422; Zuckermann, et al., 1994. J. Med. Chem. 37: 2678; Cho, et al., 1993. Science 261: 1303; Carrell, et al., 1994. Angew. Chem. Int. Ed. Engl. 33: 2059; Carell, et al., 1994. Angew. Chem. Int. Ed. Engl. 33: 2061; and Gallop, et al., 1994. J. Med. Chem. 37:1233.

Libraries of compounds may be presented in solution (e.g., Houghten, 1992. Biotechniques 13: 412-421), or on beads (Lam, 1991. Nature 354: 82-84), on chips (Fodor, 1993. Nature 364: 555-556), bacteria (Ladner, U.S. Pat. No. 5,223,409), spores (Ladner, U.S. Pat. 5,233,409), plasmids (Cull, et al., 1992. Proc. Natl. Acad. Sci. USA 89: 1865-1869) or on phage (Scott and Smith, 1990. Science 249: 386-390; Devlin, 1990. Science 249: 404-406; Cwirla, et al., 1990. Proc. Natl. Acad. Sci. U.S.A. 87: 6378-6382; Felici, 1991. J. Mol. Biol. 222: 301-310; Ladner, U.S. Pat. No. 5,233,409.).

In one embodiment, an assay is a cell-based assay in which a cell which expresses a membrane-bound form of NOVX protein, or a biologically-active portion thereof, on the cell surface is contacted with a test compound and the ability of the test compound to bind to an NOVX protein determined. The cell, for example, can of mammalian origin or a yeast cell. Determining the ability of the test compound to bind to the NOVX protein can be accomplished, for example, by coupling the test compound with a radioisotope or enzymatic label such that binding of the test compound to the NOVX protein or biologically-active portion thereof can be determined by detecting the labeled compound in a complex. For example, test compounds can be labeled with ¹²⁵I, ³⁵S, ¹⁴C, or ³H, either directly or indirectly, and the radioisotope detected by direct counting of radioemission or by scintillation counting. Alternatively, test compounds can be enzymatically-labeled with, for example, horseradish peroxidase, alkaline phosphatase, or luciferase, and the enzymatic label detected by determination of conversion of an appropriate substrate to product. In one embodiment, the assay comprises contacting a cell which expresses a membrane-bound form of NOVX protein, or a biologically-active portion thereof, on the cell surface with a known compound which binds NOVX to form an assay mixture, contacting the assay mixture with a test compound, and determining the ability of the test compound to interact with an NOVX protein, wherein determining the ability of the test compound to interact with an NOVX protein comprises determining the ability of the test compound to preferentially bind to NOVX protein or a biologically-active portion thereof as compared to the known compound.

In another embodiment, an assay is a cell-based assay comprising contacting a cell expressing a membrane-bound form of NOVX protein, or a biologically-active portion thereof, on the cell surface with a test compound and determining the ability of the test compound to modulate (e.g., stimulate or inhibit) the activity of the NOVX protein or biologically-active portion thereof. Determining the ability of the test compound to modulate the activity of NOVX or a biologically-active portion thereof can be accomplished, for example, by determining the ability of the NOVX protein to bind to or interact with an NOVX target molecule. As used herein, a “target molecule” is a molecule with which an NOVX protein binds or interacts in nature, for example, a molecule on the surface of a cell which expresses an NOVX interacting protein, a molecule on the surface of a second cell, a molecule in the extracellular milieu, a molecule associated with the internal surface of a cell membrane or a cytoplasmic molecule. An NOVX target molecule can be a non-NOVX molecule or an NOVX protein or polypeptide of the invention. In one embodiment, an NOVX target molecule is a component of a signal transduction pathway that facilitates transduction of an extracellular signal (e.g. a signal generated by binding of a compound to a membrane-bound NOVX molecule) through the cell membrane and into the cell. The target, for example, can be a second intercellular protein that has catalytic activity or a protein that facilitates the association of downstream signaling molecules with NOVX.

Determining the ability of the NOVX protein to bind to or interact with an NOVX target molecule can be accomplished by one of the methods described above for determining direct binding. In one embodiment, determining the ability of the NOVX protein to bind to or interact with an NOVX target molecule can be accomplished by determining the activity of the target molecule. For example, the activity of the target molecule can be determined by detecting induction of a cellular second messenger of the target (i.e. intracellular Ca²⁺, diacylglycerol, IP₃, etc.), detecting catalytic/enzymatic activity of the target an appropriate substrate, detecting the induction of a reporter gene (comprising an NOVX-responsive regulatory element operatively linked to a nucleic acid encoding a detectable marker, e.g., luciferase), or detecting a cellular response, for example, cell survival, cellular differentiation, or cell proliferation.

In yet another embodiment, an assay of the invention is a cell-free assay comprising contacting an NOVX protein or biologically-active portion thereof with a test compound and determining the ability of the test compound to bind to the NOVX protein or biologically-active portion thereof. Binding of the test compound to the NOVX protein can be determined either directly or indirectly as described above. In one such embodiment, the assay comprises contacting the NOVX protein or biologically-active portion thereof with a known compound which binds NOVX to form an assay mixture, contacting the assay mixture with a test compound, and determining the ability of the test compound to interact with an NOVX protein, wherein determining the ability of the test compound to interact with an NOVX protein comprises determining the ability of the test compound to preferentially bind to NOVX or biologically-active portion thereof as compared to the known compound.

In still another embodiment, an assay is a cell-free assay comprising contacting NOVX protein or biologically-active portion thereof with a test compound and determining the ability of the test compound to modulate (e.g. stimulate or inhibit) the activity of the NOVX protein or biologically-active portion thereof. Determining the ability of the test compound to modulate the activity of NOVX can be accomplished, for example, by determining the ability of the NOVX protein to bind to an NOVX target molecule by one of the methods described above for determining direct binding. In an alternative embodiment, determining the ability of the test compound to modulate the activity of NOVX protein can be accomplished by determining the ability of the NOVX protein further modulate an NOVX target molecule. For example, the catalytic/enzymatic activity of the target molecule on an appropriate substrate can be determined as described, supra.

In yet another embodiment, the cell-free assay comprises contacting the NOVX protein or biologically-active portion thereof with a known compound which binds NOVX protein to form an assay mixture, contacting the assay mixture with a test compound, and determining the ability of the test compound to interact with an NOVX protein, wherein determining the ability of the test compound to interact with an NOVX protein comprises determining the ability of the NOVX protein to preferentially bind to or modulate the activity of an NOVX target molecule.

The cell-free assays of the invention are amenable to use of both the soluble form or the membrane-bound form of NOVX protein. In the case of cell-free assays comprising the membrane-bound form of NOVX protein, it may be desirable to utilize a solubilizing agent such that the membrane-bound form of NOVX protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-1 14, Thesit®, Isotridecypoly(ethylene glycol ether)ₙ, N-dodecyl-N,N-dimethyl-3-ammonio-1-propane sulfonate, 3-(3-cholamidopropyl) dimethylamminiol-1-propane sulfonate (CHAPS), or 3-(3-cholamidopropyl)dimethylamminiol-2-hydroxy-1-propane sulfonate (CHAPSO).

In more than one embodiment of the above assay methods of the invention, it may be desirable to immobilize either NOVX protein or its target molecule to facilitate separation of complexed from uncomplexed forms of one or both of the proteins, as well as to accommodate automation of the assay. Binding of a test compound to NOVX protein, or interaction of NOVX protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided that adds a domain that allows one or both of the proteins to be bound to a matrix. For example, GST-NOVX fusion proteins or GST-target fusion proteins can be adsorbed onto glutathione sepharose beads (Sigma Chemical, St. Louis, Mo.) or glutathione derivatized microtiter plates, that are then combined with the test compound or the test compound and either the non-adsorbed target protein or NOVX protein, and the mixture is incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described, supra. Alternatively, the complexes can be dissociated from the matrix, and the level of NOVX protein binding or activity determined using standard techniques.

Other techniques for immobilizing proteins on matrices can also be used in the screening assays of the invention. For example, either the NOVX protein or its target molecule can be immobilized utilizing conjugation of biotin and streptavidin. Biotinylated NOVX protein or target molecules can be prepared from biotin-NHS (N-hydroxy-succinimide) using techniques well-known within the art (e.g., biotinylation kit, Pierce Chemicals, Rockford, Ill.), and immobilized in the wells of streptavidin-coated 96 well plates (Pierce Chemical). Alternatively, antibodies reactive with NOVX protein or target molecules, but which do not interfere with binding of the NOVX protein to its target molecule, can be derivatized to the wells of the plate, and unbound target or NOVX protein trapped in the wells by antibody conjugation. Methods for detecting such complexes, in addition to those described above for the GST-immobilized complexes, include immunodetection of complexes using antibodies reactive with the NOVX protein or target molecule, as well as enzyme-linked assays that rely on detecting an enzymatic activity associated with the NOVX protein or target molecule.

In another embodiment, modulators of NOVX protein expression are identified in a method wherein a cell is contacted with a candidate compound and the expression of NOVX mRNA or protein in the cell is determined. The level of expression of NOVX mRNA or protein in the presence of the candidate compound is compared to the level of expression of NOVX mRNA or protein in the absence of the candidate compound. The candidate compound can then be identified as a modulator of NOVX mRNA or protein expression based upon this comparison. For example, when expression of NOVX mRNA or protein is greater (i.e., statistically significantly greater) in the presence of the candidate compound than in its absence, the candidate compound is identified as a stimulator of NOVX mRNA or protein expression. Alternatively, when expression of NOVX mRNA or protein is less (statistically significantly less) in the presence of the candidate compound than in its absence, the candidate compound is identified as an inhibitor of NOVX mRNA or protein expression. The level of NOVX mRNA or protein expression in the cells can be determined by methods described herein for detecting NOVX mRNA or protein.

In yet another aspect of the invention, the NOVX proteins can be used as “bait proteins” in a two-hybrid assay or three hybrid assay (see, e.g., U.S. Pat. No. 5,283,317; Zervos, et al., 1993. Cell 72: 223-232; Madura, et al., 1993. J. Biol. Chem. 268: 12046-12054; Bartel, et al., 1993. Biotechniques 14: 920-924; Iwabuchi, et al., 1993. Oncogene 8: 1693-1696; and Brent WO 94/10300), to identify other proteins that bind to or interact with NOVX (“NOVX-binding proteins” or “NOVX-bp”) and modulate NOVX activity. Such NOVX-binding proteins are also likely to be involved in the propagation of signals by the NOVX proteins as, for example, upstream or downstream elements of the NOVX pathway.

The two-hybrid system is based on the modular nature of most transcription factors, which consist of separable DNA-binding and activation domains. Briefly, the assay utilizes two different DNA constructs. In one construct, the gene that codes for NOVX is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming an NOVX-dependent complex, the DNA-binding and activation domains of the transcription factor are brought into close proximity. This proximity allows transcription of a reporter gene (e.g., LacZ) that is operably linked to a transcriptional regulatory site responsive to the transcription factor. Expression of the reporter gene can be detected and cell colonies containing the functional transcription factor can be isolated and used to obtain the cloned gene that encodes the protein which interacts with NOVX.

The invention further pertains to novel agents identified by the aforementioned screening assays and uses thereof for treatments as described herein.

Detection Assays

Portions or fragments of the cDNA sequences identified herein (and the corresponding complete gene sequences) can be used in numerous ways as polynucleotide reagents. By way of example, and not of limitation, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. Some of these applications are described in the subsections, below.

Chromosome Mapping

Once the sequence (or a portion of the sequence) of a gene has been isolated, this sequence can be used to map the location of the gene on a chromosome. This process is called chromosome mapping. Accordingly, portions or fragments of the NOVX sequences, SEQ ID NOS: 1, 3, 5, 7, 9, 11, 13, 15, 17, 19, 21, 23, 25, 27, 29, and 31, or fragments or derivatives thereof, can be used to map the location of the NOVX genes, respectively, on a chromosome. The mapping of the NOVX sequences to chromosomes is an important first step in correlating these sequences with genes associated with disease.

Briefly, NOVX genes can be mapped to chromosomes by preparing PCR primers (preferably 15-25 bp in length) from the NOVX sequences. Computer analysis of the NOVX, sequences can be used to rapidly select primers that do not span more than one exon in the genomic DNA, thus complicating the amplification process. These primers can then be used for PCR screening of somatic cell hybrids containing individual human chromosomes. Only those hybrids containing the human gene corresponding to the NOVX sequences will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow, because they lack a particular enzyme, but in which human cells can, the one human chromosome that contains the gene encoding the needed enzyme will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes. See, e.g., D'Eustachio, et al., 1983. Science 220: 919-924. Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycler. Using the NOVX sequences to design oligonucleotide primers, sub-localizatiion can be achieved with panels of fragments from specific chromosomes.

Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical like colcemid that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases, will suffice to get good results at a reasonable amount of time. For a review of this technique, see, Verma, et al., HUMAN CHROMOSOMES: A MANUAL OF BASIC TECHNIQUES (Pergamon Press, New York 1988).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. Such data are found, e.g., in McKusick, MENDELIAN INHERITANCE IN MAN, available on-line through Johns Hopkins University Welch Medical Library). The relationship between genes and disease, mapped to the same chromosomal region, can then be identified through linkage analysis (co-inheritance of physically adjacent genes), described in, e.g., Egeland, et al., 1987. Nature, 325: 783-787.

Moreover, differences in the DNA sequences between individuals affected and unaffected with a disease associated with the NOVX gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

Tissue Typing

The NOVX sequences of the invention can also be used to identify individuals from minute biological samples. In this technique, an individual's genomic DNA is digested with one or more restriction enzymes, and probed on a Southern blot to yield unique bands for identification. The sequences of the invention are useful as additional DNA markers for RFLP (“restriction fragment length polymorphisms,” described in U.S. Pat. No. 5,272,057).

Furthermore, the sequences of the invention can be used to provide an alternative technique that determines the actual base-by-base DNA sequence of selected portions of an individual's genome. Thus, the NOVX sequences described herein can be used to prepare two PCR primers from the 5′-and 3′-termini of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the invention can be used to obtain such identification sequences from individuals and from tissue. The NOVX sequences of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Much of the allelic variation is due to single nucleotide polymorphisms (SNPs), which include restriction fragment length polymorphisms (RFLPs).

Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences can comfortably provide positive individual identification with a panel of perhaps 10 to 1,000 primers that each yield a noncoding amplified sequence of 100 bases. If predicted coding sequences, such as those in SEQ ID NOS: 1, 3, 5, 7, 9, 11, 13, 15, 17, 19, 21, 23, 25, 27, 29, and 31 are used, a more appropriate number of primers for positive individual identification would be 500-2,000.

Predictive Medicine

The invention also pertains to the field of predictive medicine in which diagnostic assays, prognostic assays, pharmacogenomics, and monitoring clinical trials are used for prognostic (predictive) purposes to thereby treat an individual prophylactically. Accordingly, one aspect of the invention relates to diagnostic assays for determining NOVX protein and/or nucleic acid expression as well as NOVX activity, in the context of a biological sample (e.g., blood, serum, cells, tissue) to thereby determine whether an individual is afflicted with a disease or disorder, or is at risk of developing a disorder, associated with aberrant NOVX expression or activity. The disorders include metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders, and the various dyslipidemias, metabolic disturbances associated with obesity, the metabolic syndrome X and wasting disorders associated with chronic diseases and various cancers. The invention also provides for prognostic (or predictive) assays for determining whether an individual is at risk of developing a disorder associated with NOVX protein, nucleic acid expression or activity. For example, mutations in an NOVX gene can be assayed in a biological sample. Such assays can be used for prognostic or predictive purpose to thereby prophylactically treat an individual prior to the onset of a disorder characterized by or associated with NOVX protein, nucleic acid expression, or biological activity.

Another aspect of the invention provides methods for determining NOVX protein, nucleic acid expression or activity in an individual to thereby select appropriate therapeutic or prophylactic agents for that individual (referred to herein as “pharmacogenomics”). Pharmacogenomics allows for the selection of agents (e.g., drugs) for therapeutic or prophylactic treatment of an individual based on the genotype of the individual (e.g., the genotype of the individual examined to determine the ability of the individual to respond to a particular agent.)

Yet another aspect of the invention pertains to monitoring the influence of agents (e.g., drugs, compounds) on the expression or activity of NOVX in clinical trials.

These and other agents are described in further detail in the following sections.

Diagnostic Assays

An exemplary method for detecting the presence or absence of NOVX in a biological sample involves obtaining a biological sample from a test subject and contacting the biological sample with a compound or an agent capable of detecting NOVX protein or nucleic acid (e.g., mRNA, genomic DNA) that encodes NOVX protein such that the presence of NOVX is detected in the biological sample. An agent for detecting NOVX mRNA or genomic DNA is a labeled nucleic acid probe capable of hybridizing to NOVX mRNA or genomic DNA. The nucleic acid probe can be, for example, a full-length NOVX nucleic acid, such as the nucleic acid of SEQ ID NOS: 1, 3, 5, 7, 9, 11, 13, 15, 17, 19, 21, 23, 25, 27, 29, and 31, or a portion thereof, such as an oligonucleotide of at least 15, 30, 50, 100, 250 or 500 nucleotides in length and sufficient to specifically hybridize under stringent conditions to NOVX mRNA or genomic DNA. Other suitable probes for use in the diagnostic assays of the invention are described herein.

An agent for detecting NOVX protein is an antibody capable of binding to NOVX protein, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. That is, the detection method of the invention can be used to detect NOVX mRNA, protein, or genomic DNA in a biological sample in vitro as well as in vivo. For example, in vitro techniques for detection of NOVX mRNA include Northern hybridizations and in situ hybridizations. In vitro techniques for detection of NOVX protein include enzyme linked immunosorbent assays (ELISAs), Western blots, immunoprecipitations, and immunofluorescence. In vitro techniques for detection of NOVX genomic DNA include Southern hybridizations. Furthermore, in vivo techniques for detection of NOVX protein include introducing into a subject a labeled anti-NOVX antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a peripheral blood leukocyte sample isolated by conventional means from a subject.

In another embodiment, the methods further involve obtaining a control biological sample from a control subject, contacting the control sample with a compound or agent capable of detecting NOVX protein, mRNA, or genomic DNA, such that the presence of NOVX protein, mRNA or genomic DNA is detected in the biological sample, and comparing the presence of NOVX protein, mRNA or genomic DNA in the control sample with the presence of NOVX protein, mRNA or genomic DNA in the test sample.

The invention also encompasses kits for detecting the presence of NOVX in a biological sample. For example, the kit can comprise: a labeled compound or agent capable of detecting NOVX protein or mRNA in a biological sample; means for determining the amount of NOVX in the sample; and means for comparing the amount of NOVX in the sample with a standard. The compound or agent can be packaged in a suitable container. The kit can further comprise instructions for using the kit to detect NOVX protein or nucleic acid.

Prognostic Assays

The diagnostic methods described herein can furthermore be utilized to identify subjects having or at risk of developing a disease or disorder associated with aberrant NOVX expression or activity. For example, the assays described herein, such as the preceding diagnostic assays or the following assays, can be utilized to identify a subject having or at risk of developing a disorder associated with NOVX protein, nucleic acid expression or activity. Alternatively, the prognostic assays can be utilized to identify a subject having or at risk for developing a disease or disorder. Thus, the invention provides a method for identifying a disease or disorder associated with aberrant NOVX expression or activity in which a test sample is obtained from a subject and NOVX protein or nucleic acid (e.g., mRNA, genomic DNA) is detected, wherein the presence of NOVX protein or nucleic acid is diagnostic for a subject having or at risk of developing a disease or disorder associated with aberrant NOVX expression or activity. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant NOVX expression or activity. For example, such methods can be used to determine whether a subject can be effectively treated with an agent for a disorder. Thus, the invention provides methods for determining whether a subject can be effectively treated with an agent for a disorder associated with aberrant NOVX expression or activity in which a test sample is obtained and NOVX protein or nucleic acid is detected (e.g., wherein the presence of NOVX protein or nucleic acid is diagnostic for a subject that can be administered the agent to treat a disorder associated with aberrant NOVX expression or activity).

The methods of the invention can also be used to detect genetic lesions in an NOVX gene, thereby determining if a subject with the lesioned gene is at risk for a disorder characterized by aberrant cell proliferation and/or differentiation. In various embodiments, the methods include detecting, in a sample of cells from the subject, the presence or absence of a genetic lesion characterized by at least one of an alteration affecting the integrity of a gene encoding an NOVX-protein, or the misexpression of the NOVX gene. For example, such genetic lesions can be detected by ascertaining the existence of at least one of: (i) a deletion of one or more nucleotides from an NOVX gene; (ii) an addition of one or more nucleotides to an NOVX gene; (iii) a substitution of one or more nucleotides of an NOVX gene, (iv) a chromosomal rearrangement of an NOVX gene; (v) an alteration in the level of a messenger RNA transcript of an NOVX gene, (vi) aberrant modification of an NOVX gene, such as of the methylation pattern of the genomic DNA, (vii) the presence of a non-wild-type splicing pattern of a messenger RNA transcript of an NOVX gene, (viii) a non-wild-type level of an NOVX protein, (ix) allelic loss of an NOVX gene, and (x) inappropriate post-translational modification of an NOVX protein. As described herein, there are a large number of assay techniques known in the art which can be used for detecting lesions in an NOVX gene. A preferred biological sample is a peripheral blood leukocyte sample isolated by conventional means from a subject. However, any biological sample containing nucleated cells may be used, including, for example, buccal mucosal cells.

In certain embodiments, detection of the lesion involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran, et al., 1988. Science 241: 1077-1080; and Nakazawa, et al., 1994. Proc. Natl. Acad. Sci. USA 91: 360-364), the latter of which can be particularly useful for detecting point mutations in the NOVX-gene (see, Abravaya, et al., 1995. Nucl. Acids Res. 23: 675-682). This method can include the steps of collecting a sample of cells from a patient, isolating nucleic acid (e.g., genomic, mRNA or both) from the cells of the sample, contacting the nucleic acid sample with one or more primers that specifically hybridize to an NOVX gene under conditions such that hybridization and amplification of the NOVX gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (see, Guatelli, et al., 1990. Proc. Natl. Acad. Sci. USA 87: 1874-1878), transcriptional amplification system (see, Kwoh, et al., 1989. Proc. Natl. Acad. Sci. USA 86: 1173-1177); Qβ Replicase (see, Lizardi, et al, 1988. BioTechnology 6: 1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in an NOVX gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, e.g., U.S. Pat. No. 5,493,531) can be used to score for the presence of specific mutations by development or loss of a ribozyme cleavage site.

In other embodiments, genetic mutations in NOVX can be identified by hybridizing a sample and control nucleic acids, e.g., DNA or RNA, to high-density arrays containing hundreds or thousands of oligonucleotides probes. See, e.g., Cronin, et al., 1996. Human Mutation 7: 244-255; Kozal, et al., 1996. Nat. Med. 2: 753-759. For example, genetic mutations in NOVX can be identified in two dimensional arrays containing light-generated DNA probes as described in Cronin, et al., supra. Briefly, a first hybridization array of probes can be used to scan through long stretches of DNA in a sample and control to identify base changes between the sequences by making linear arrays of sequential overlapping probes. This step allows the identification of point mutations. This is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the NOVX gene and detect mutations by comparing the sequence of the sample NOVX with the corresponding wild-type (control) sequence. Examples of sequencing reactions include those based on techniques developed by Maxim and Gilbert, 1977. Proc. Natl. Acad. Sci. USA 74: 560 or Sanger, 1977. Proc. Natl. Acad. Sci. USA 74: 5463. It is also contemplated that any of a variety of automated sequencing procedures can be utilized when performing the diagnostic assays (see, e.g., Naeve, et al., 1995. Biotechniques 19: 448), including sequencing by mass spectrometry (see, e.g., PCT International Publication No. WO 94/16101; Cohen, et al., 1996. Adv. Chromatography 36: 127-162; and Griffin, et al., 1993. Appl. Biochem. Biotechnol. 38: 147-159).

Other methods for detecting mutations in the NOVX gene include methods in which protection from cleavage agents is used to detect mismatched bases in RNA/RNA or RNA/DNA heteroduplexes. See, e.g., Myers, et al., 1985. Science 230: 1242. In general, the art technique of “mismatch cleavage” starts by providing heteroduplexes of formed by, hybridizing (labeled) RNA or DNA containing the wild-type NOVX sequence with potentially mutant RNA or DNA obtained from a tissue sample. The double-stranded duplexes are treated with an agent that cleaves single-stranded regions of the duplex such as which will exist due to basepair mismatches between the control and sample strands. For instance, RNA/DNA duplexes can be treated with RNase and DNA/DNA hybrids treated with S₁ nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, e.g., Cotton, et al., 1988. Proc. Natl. Acad. Sci. USA 85: 4397; Saleeba, et al., 1992. Methods Enzymol. 217: 286-295. In an embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in NOVX cDNAs obtained from samples of cells. For example, the mutY enzyme of E. coli cleaves A at G/A mismatches and the thymidine DNA glycosylase from HeLa cells cleaves T at G/T mismatches. See, e.g., Hsu, et al., 1994. Carcinogenesis 15: 1657-1662. According to an exemplary embodiment, a probe based on an NOVX sequence, e.g., a wild-type NOVX sequence, is hybridized to a cDNA or other DNA product from a test cell(s). The duplex is treated with a DNA mismatch repair enzyme, and the cleavage products, if any, can be detected from electrophoresis protocols or the like. See, e.g., U.S. Pat. No. 5,459,039.

In other embodiments, alterations in electrophoretic mobility will be used to identify mutations in NOVX genes. For example, single strand conformation polymorphism (SSCP) may be used to detect differences in electrophoretic mobility between mutant and wild -type nucleic acids. See, e.g., Orita, et al., 1989. Proc. Natl. Acad. Sci. USA: 86: 2766; Cotton, 1993. Mutat. Res. 285: 125-144; Hayashi, 1992. Genet. Anal. Tech. Appl. 9: 73-79. Single-stranded DNA fragments of sample and control NOVX nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In one embodiment, the subject method utilizes heteroduplex analysis to separate double stranded heteroduplex molecules on the basis of changes in electrophoretic mobility. See, e.g., Keen, et al., 1991. Trends Genet. 7: 5.

In yet another embodiment, the movement of mutant or wild-type fragments in polyacrylamide gels containing a gradient of denaturant is assayed using denaturing gradient gel electrophoresis (DGGE). See, e.g., Myers, et al., 1985. Nature 313: 495. When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA. See, e.g., Rosenbaum and Reissner, 1987. Biophys. Chem. 265: 12753.

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions that permit hybridization only if a perfect match is found. See, e.g., Saiki, et al., 1986. Nature 324: 163; Saiki, et al., 1989. Proc. Natl. Acad. Sci. USA 86: 6230. Such allele specific oligonucleotides are hybridized to PCR amplified target DNA or a number of different mutations when the oligonucleotides are attached to the hybridizing membrane and hybridized with labeled target DNA.

Alternatively, allele specific amplification technology that depends on selective PCR amplification may be used in conjunction with the instant invention. Oligonucleotides used as primers for specific amplification may carry the mutation of interest in the center of the molecule (so that amplification depends on differential hybridization; see, e.g., Gibbs, et al., 1989. Nucl. Acids Res. 17: 2437-2448) or at the extreme 3′-terminus of one primer where, under appropriate conditions, mismatch can prevent, or reduce polymerase extension (see, e.g., Prossner, 1993. Tibtech. 11: 238). In addition it may be desirable to introduce a novel restriction site in the region of the mutation to create cleavage-based detection. See, e.g., Gasparini, et al., 1992. Mol. Cell Probes 6: 1. It is anticipated that in certain embodiments amplification may also be performed using Taq ligase for amplification. See, e.g., Barany, 1991. Proc. Natl. Acad. Sci. USA 88: 189. In such cases, ligation will occur only if there is a perfect match at the 3′-terminus of the 5′ sequence, making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving an NOVX gene.

Furthermore, any cell type or tissue, preferably peripheral blood leukocytes, in which NOVX is expressed may be utilized in the prognostic assays described herein. However, any biological sample containing nucleated cells may be used, including, for example, buccal mucosal cells.

Pharmacogenomics

Agents, or modulators that have a stimulatory or inhibitory effect on NOVX activity (e.g., NOVX gene expression), as identified by a screening assay described herein can be administered to individuals to treat (prophylactically or therapeutically) disorders (The disorders include metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders, and the various dyslipidemias, metabolic disturbances associated with obesity, the metabolic syndrome X and wasting disorders associated with chronic diseases and various cancers.) In conjunction with such treatment, the pharmacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the activity of NOVX protein, expression of NOVX nucleic acid, or mutation content of NOVX genes in an individual can be determined to thereby select appropriate agent(s) for therapeutic or prophylactic treatment of the individual.

Pharmacogenomics deals with clinically significant hereditary variations in the response to drugs due to altered drug disposition and abnormal action in affected persons. See e.g., Eichelbaum, 1996. Clin. Exp. Pharmacol. Physiol., 23: 983-985; Linder, 1997. Clin. Chem., 43: 254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare defects or as polymorphisms. For example, glucose-6-phosphate dehydrogenase (G6PD) deficiency is a common inherited enzymopathy in which the main clinical complication is hemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. At the other extreme are the so called ultra-rapid metabolizers who do not respond to standard doses. Recently, the molecular basis of ultra-rapid metabolism has been identified to be due to CYP2D6 gene amplification.

Thus, the activity of NOVX protein, expression of NOVX nucleic acid, or mutation content of NOVX genes in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with an NOVX modulator, such as a modulator identified by one of the exemplary screening assays described herein.

Monitoring of Effects During Clinical Trials

Monitoring the influence of agents (e.g., drugs, compounds) on the expression or activity of NOVX (e.g., the ability to modulate aberrant cell proliferation and/or differentiation) can be applied not only in basic drug screening, but also in clinical trials. For example, the effectiveness of an agent determined by a screening assay as described herein to increase NOVX gene expression, protein levels, or upregulate NOVX activity, can be monitored in clinical trails of subjects exhibiting decreased NOVX gene expression, protein levels, or downregulated NOVX activity. Alternatively, the effectiveness of an agent determined by a screening assay to decrease NOVX gene expression, protein levels, or downregulate NOVX activity, can be monitored in clinical trails of subjects exhibiting increased NOVX gene expression, protein levels, or upregulated NOVX activity. In such clinical trials, the expression or activity of NOVX and, preferably, other genes that have been implicated in, for example, a cellular proliferation or immune disorder can be used as a “read out” or markers of the immune responsiveness of a particular cell.

By way of example, and not of limitation, genes, including NOVX, that are modulated in cells by treatment with an agent (e.g., compound, drug or small molecule) that modulates NOVX activity (e.g., identified in a screening assay as described herein) can be identified. Thus, to study the effect of agents on cellular proliferation disorders, for example, in a clinical trial, cells can be isolated and RNA prepared and analyzed for the levels of expression of NOVX and other genes implicated in the disorder. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of NOVX or other genes. In this manner, the gene expression pattern can serve as a marker, indicative of the physiological response of the cells to the agent. Accordingly, this response state may be determined before, and at various points during, treatment of the individual with the agent.

In one embodiment, the invention provides a method for monitoring the effectiveness of treatment of a subject with an agent (e.g., an agonist, antagonist, protein, peptide, peptidomimetic, nucleic acid, small molecule, or other drug candidate identified by the screening assays described herein) comprising the steps of (i) obtaining a pre-administration sample from a subject prior to administration of the agent; (ii) detecting the level of expression of an NOVX protein, mRNA, or genomic DNA in the preadministration sample; (iii) obtaining one or more post-administration samples from the subject; (iv) detecting the level of expression or activity of the NOVX protein, mRNA, or genomic DNA in the post-administration samples; (v) comparing the level of expression or activity of the NOVX protein, mRNA, or genomic DNA in the pre-administration sample with the NOVX protein, mRNA, or genomic DNA in the post administration sample or samples; and (vi) altering the administration of the agent to the subject accordingly. For example, increased administration of the agent may be desirable to increase the expression or activity of NOVX to higher levels than detected, i.e., to increase the effectiveness of the agent. Alternatively, decreased administration of the agent may be desirable to decrease expression or activity of NOVX to lower levels than detected, i.e., to decrease the effectiveness of the agent.

Methods of Treatment

The invention provides for both prophylactic and therapeutic methods of treating a subject at risk of (or susceptible to) a disorder or having a disorder associated with aberrant NOVX expression or activity. The disorders include cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, obesity, transplantation, adrenoleukodystrophy, congenital adrenal hyperplasia, prostate cancer, neoplasm; adenocarcinoma, lymphoma, uterus cancer, fertility, hemophilia, hypercoagulation, idiopathic thrombocytopenic purpura, immunodeficiencies, graft versus host disease, AIDS, bronchial asthma, Crohn's disease; multiple sclerosis, treatment of Albright Hereditary Ostoeodystrophy, and other diseases, disorders and conditions of the like.

These methods of treatment will be discussed more fully, below.

Disease and Disorders

Diseases and disorders that are characterized by increased (relative to a subject not suffering from the disease or disorder) levels or biological activity may be treated with Therapeutics that antagonize (i.e., reduce or inhibit) activity. Therapeutics that antagonize activity may be administered in a therapeutic or prophylactic manner. Therapeutics that may be utilized include, but are not limited to: (i) an aforementioned peptide, or analogs, derivatives, fragments or homologs thereof; (ii) antibodies to an aforementioned peptide; (iii) nucleic acids encoding an aforementioned peptide; (iv) administration of antisense nucleic acid and nucleic acids that are “dysfunctional” (i.e., due to a heterologous insertion within the coding sequences of coding sequences to an aforementioned peptide) that are utilized to “knockout” endogenous function of an aforementioned peptide by homologous recombination (see, e.g., Capecchi, 1989. Science 244: 1288-1292); or (v) modulators ( i.e., inhibitors, agonists and antagonists, including additional peptide mimetic of the invention or antibodies specific to a peptide of the invention) that alter the interaction between an aforementioned peptide and its binding partner.

Diseases and disorders that are characterized by decreased (relative to a subject not suffering from the disease or disorder) levels or biological activity may be treated with Therapeutics that increase (i.e., are agonists to) activity. Therapeutics that upregulate activity may be administered in a therapeutic or prophylactic manner. Therapeutics that may be utilized include, but are not limited to, an aforementioned peptide, or analogs, derivatives, fragments or homologs thereof; or an agonist that increases bioavailability.

Increased or decreased levels can be readily detected by quantifying peptide and/or RNA, by obtaining a patient tissue sample (e.g., from biopsy tissue) and assaying it in vitro for RNA or peptide levels, structure and/or activity of the expressed peptides (or mRNAs of an aforementioned peptide). Methods that are well-known within the art include, but are not limited to, immunoassays (e.g., by Western blot analysis, immunoprecipitation followed by sodium dodecyl sulfate (SDS) polyacrylamide gel electrophoresis, immunocytochemistry, etc.) and/or hybridization assays to detect expression of mRNAs (e.g., Northern assays, dot blots, in situ hybridization, and the like).

Prophylactic Methods

In one aspect, the invention provides a method for preventing, in a subject, a disease or condition associated with an aberrant NOVX expression or activity, by administering to the subject an agent that modulates NOVX expression or at least one NOVX activity. Subjects at risk for a disease that is caused or contributed to by aberrant NOVX expression or activity can be identified by, for example, any or a combination of diagnostic or prognostic assays as described herein. Administration of a prophylactic agent can occur prior to the manifestation of symptoms characteristic of the NOVX aberrancy, such that a disease or disorder is prevented or, alternatively, delayed in its progression. Depending upon the type of NOVX aberrancy, for example, an NOVX agonist or NOVX antagonist agent can be used for treating the subject. The appropriate agent can be determined based on screening assays described herein. The prophylactic methods of the invention are further discussed in the following subsections.

Therapeutic Methods

Another aspect of the invention pertains to methods of modulating NOVX expression or activity for therapeutic purposes. The modulatory method of the invention involves contacting a cell with an agent that modulates one or more of the activities of NOVX protein activity associated with the cell. An agent that modulates NOVX protein activity can be an agent as described herein, such as a nucleic acid or a protein, a naturally-occurring cognate ligand of an NOVX protein, a peptide, an NOVX peptidomimetic, or other small molecule. In one embodiment, the agent stimulates one or more NOVX protein activity. Examples of such stimulatory agents include active NOVX protein and a nucleic acid molecule encoding NOVX that has been introduced into the cell. In another embodiment, the agent inhibits one or more NOVX protein activity. Examples of such inhibitory agents include antisense NOVX nucleic acid molecules and anti-NOVX antibodies. These modulatory methods can be performed in vitro (e.g., by culturing the cell with the agent) or, alternatively, in vivo (e.g., by administering the agent to a subject). As such, the invention provides methods of treating an individual afflicted with a disease or disorder characterized by aberrant expression or activity of an NOVX protein or nucleic acid molecule. In one embodiment, the method involves administering an agent (e.g., an agent identified by a screening assay described herein), or combination of agents that modulates (e.g., up-regulates or down-regulates) NOVX expression or activity. In another embodiment, the method involves administering an NOVX protein or nucleic acid molecule as therapy to compensate for reduced or aberrant NOVX expression or activity.

Stimulation of NOVX activity is desirable in situations in which NOVX is abnormally downregulated and/or in which increased NOVX activity is likely to have a beneficial effect. One example of such a situation is where a subject has a disorder characterized by aberrant cell proliferation and/or differentiation (e.g., cancer or immune associated disorders). Another example of such a situation is where the subject has a gestational disease (e.g., preclampsia).

Determination of the Biological Effect of the Therapeutic

In various embodiments of the invention, suitable in vitro or in vivo assays are performed to determine the effect of a specific Therapeutic and whether its administration is indicated for treatment of the affected tissue.

In various specific embodiments, in vitro assays may be performed with representative cells of the type(s) involved in the patient's disorder, to determine if a given Therapeutic exerts the desired effect upon the cell type(s). Compounds for use in therapy may be tested in suitable animal model systems including, but not limited to rats, mice, chicken, cows, monkeys, rabbits, and the like, prior to testing in human subjects. Similarly, for in vivo testing, any of the animal model system known in the art may be used prior to administration to human subjects.

Prophylactic and Therapeutic Uses of the Compositions of the Invention

The NOVX nucleic acids and proteins of the invention are useful in potential prophylactic and therapeutic applications implicated in a variety of disorders including, but not limited to: metabolic disorders, diabetes, obesity, infectious disease, anorexia, cancer-associated cancer, neurodegenerative disorders, Alzheimer's Disease, Parkinson's Disorder, immune disorders, hematopoietic disorders, and the various dyslipidemias, metabolic disturbances associated with obesity, the metabolic syndrome X and wasting disorders associated with chronic diseases and various cancers.

As an example, a cDNA encoding the NOVX protein of the invention may be useful in gene therapy, and the protein may be useful when administered to a subject in need thereof. By way of non-limiting example, the compositions of the invention will have efficacy for treatment of patients suffering from: metabolic disorders, diabetes, obesity, infectious disease, anorexia, cancer-associated cachexia, cancer, neurodegenerative disorders, Alzheimer's Disease, Parkinson's Disorder, immune disorders, hematopoietic disorders, and the various dyslipidemias.

Both the novel nucleic acid encoding the NOVX protein, and the NOVX protein of the invention, or fragments thereof, may also be useful in diagnostic applications, wherein the presence or amount of the nucleic acid or the protein are to be assessed. A further use could be as an anti-bacterial molecule (i.e., some peptides have been found to possess anti-bacterial properties). These materials are further useful in the generation of antibodies, which immunospecifically-bind to the novel substances of the invention for use in therapeutic or diagnostic methods.

The invention will be further described in the following examples, which do not limit the scope of the invention described in the claims.

### EXAMPLES

### Example 1

Identification of NOVX Clones

The novel NOVX target sequences identified in the present invention were subjected to the exon linking process to confirm the sequence. PCR primers were designed by starting at the most upstream sequence available, for the forward primer, and at the most downstream sequence available for the reverse primer. Table 13A shows the sequences of the PCR primers used for obtaining different clones. In each case, the sequence was examined, walking inward from the respective termini toward the coding sequence, until a suitable sequence that is either unique or highly selective was encountered, or, in the case of the reverse primer, until the stop codon was reached. Such primers were designed based on in silico predictions for the full length cDNA, part (one or more exons) of the DNA or protein sequence of the target sequence, or by translated homology of the predicted exons to closely related human sequences from other species. These primers were then employed in PCR amplification based on the following pool of human cDNAs: adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea, uterus. Usually the resulting amplicons were gel purified, cloned and sequenced to high redundancy. The PCR product derived from exon linking was cloned into the pCR2.1 vector from Invitrogen. The resulting bacterial clone has an insert covering the entire open reading frame cloned into the pCR2.1 vector. Table 13B shows a list of these bacterial clones. The resulting sequences from all clones were assembled with themselves, with other fragments in CuraGen Corporation's database and with public ESTs. Fragments and ESTs were included as components for an assembly when the extent of their identity with another component of the assembly was at least 95% over 50 bp. In addition, sequence traces were evaluated manually and edited for corrections if appropriate. These procedures provide the sequence reported herein.

TABLE 13A

PCR Primers for Exon Linking

| NOVX | | SEQ ID | | SEQ ID |
|---|---|---|---|---|
| Clone | Primer 1 (5′-3′) | NO | Primer 2 (5′-3′) | NO |
| NOV1a | AGACTGGGGCCAGGGAGACAG | 119 | CAGAGGCCAAACATCCCCATCAG | 120 |
| NOV1c | GAGACAGCCCTGGGGGAGA | 121 | ACCTGCCTCCTGCCAGTCC | 122 |
| NOV6 | CATGTCCTCGACCGAGAGCGC | 123 | AGGTGGGGGGCTGCTTACTGCTT | 124 |
| NOV9a | GTCATGAAGGGGTTGCTG | 125 | GGTCAGCCCAGCCCCTCTG | 126 |
| NOV10 | CGGCTGCTGGCATGGGTG | 127 | CTCCTGCTCTGTTTCCCCCTTCAT | 128 |
| NOV11a | GCCATGGTGCTGCTGCTGCT | 129 | GGCTCAGTCGGGGTAGATGATAAAGC | 130 |
| NOV11b | CGGGCGCGGCCGTCGGAGT | 131 | CGGGGCCGGCTCAGTCGGGGTAGATGAT | 132 |

Physical clone: Exons were predicted by homology and the intron/exon boundaries were determined using standard genetic rules. Exons were further selected and refined by means of similarity determination using multiple BLAST (for example, tBlastN, BlastX, and BlastN) searches, and, in some instances, GeneScan and Grail. Expressed sequences from both public and proprietary databases were also added when available to further define and complete the gene sequence. The DNA sequence was then manually corrected for apparent inconsistencies thereby obtaining the sequences encoding the full-length protein.

TABLE 13B

Physical Clones for PCR products

| | |
|---|---|
| NOVX Clone | Clone |

| | | |
|---|---|---|
| NOV1a | Proprietary clones: | 145150175, 145150395, 145150392, 145145203, |
| | | 145150171, 145150168, 137114011 |
| NOV2a | Physical clones: | 107029754, AC078825, AC083812 |
| NOV3 | Physical clones: | 134899552, AC005230 |
| NOV4 | Genomic clnoe: | ba568g11 |
| NOV5 | Genomic clone: | AC008774 |
| NOV6 | Bacterial clone: | 111865::GMAC073364_A.698299.A2 |
| NOV7 | Physical clone: | 106973211, AC015855.4 |
| NOV8 | Physical clone: | 88091010, AL109932.3, AL360269.3, AL356323.6 |
| NOV10 | Proprietary clones: | 140488852, 133419352, 141920635 |
| NOV11a | Genomic clone: | AC026125 |
| NOV12 | Genomic clone: | AC011199 |

### Example 2

Quantitative Expression Analysis of Clones in Various Tissues and Cells

The quantitative expression of various clones was assessed using microtiter plates containing RNA samples from a variety of normal and pathology-derived cells, cell lines and tissues using real time quantitative PCR (RTQ PCR). RTQ PCR was performed on an Applied Biosystems ABI PRISM® 7700 or an ABI PRISM® 7900 HT Sequence Detection System. Various collections of samples are assembled on the plates, and referred to as Panel 1 (containing normal tissues and cancer cell lines), Panel 2 (containing samples derived from tissues from normal and cancer sources), Panel 3 (containing cancer cell lines), Panel 4 (containing cells and cell lines from normal tissues and cells related to inflammatory conditions), Panel 5D/5I (containing human tissues and cell lines with an emphasis on metabolic diseases), AI_comprehensive_panel (containing normal tissue and samples from autoinflammatory diseases), Panel CNSD.01 (containing samples from normal and diseased brains) and CNS_neurodegeneration_panel (containing samples from normal and Alzheimer's diseased brains).

RNA integrity from all samples is controlled for quality by visual assessment of agarose gel electropherograms using 28S and 18S ribosomal RNA staining intensity ratio as a guide (2:1 to 2.5:1 28s: 18s) and the absence of low molecular weight RNAs that would be indicative of degradation products. Samples are controlled against genomic DNA contamination by RTQ PCR reactions run in the absence of reverse transcriptase using probe and primer sets designed to amplify across the span of a single exon.

First, the RNA samples were normalized to reference nucleic acids such as constitutively expressed genes (for example, β-actin and GAPDH). Normalized RNA (5 ul) was converted to cDNA and analyzed by RTQ-PCR using One Step RT-PCR Master Mix Reagents (Applied Biosystems; Catalog No. 4309169) and gene-specific primers according to the manufacturer's instructions.

In other cases, non-normalized RNA samples were converted to single strand cDNA (sscDNA) using Superscript II (Invitrogen Corporation; Catalog No. 18064-147) and random hexamers according to the manufacturer's instructions. Reactions containing up to 10 μg of total RNA were performed in a volume of 20 μl and incubated for 60 minutes at 42° C. This reaction can be scaled up to 50 μg of total RNA in a final volume of 100 μl. sscDNA samples are then normalized to reference nucleic acids as described previously, using 1X TaqMan® Universal Master mix (Applied Biosystems; catalog No. 4324020), following the manufacturer's instructions.

Probes and primers were designed for each assay according to Applied Biosystems Primer Express Software package (version I for Apple Computer's Macintosh Power PC) or a similar algorithm using the target sequence as input. Default settings were used for reaction conditions and the following parameters were set before selecting primers: primer concentration=250 nM, primer melting temperature (Tm) range=58°-60° C., primer optimal Tm=59° C., maximum primer difference=2° C., probe does not have 5′G, probe Tm must be 10° C. greater than primer Tm, amplicon size 75 bp to 100 bp. The probes and primers selected (see below) were synthesized by Synthegen (Houston, Tex., USA). Probes were double purified by HPLC to remove uncoupled dye and evaluated by mass spectroscopy to verify coupling of reporter and quencher dyes to the 5′ and 3′ ends of the probe, respectively. Their final concentrations were: forward and reverse primers, 900 nM each, and probe, 200 nM.

PCR conditions: When working with RNA samples, normalized RNA from each tissue and each cell line was spotted in each well of either a 96 well or a 384-well PCR plate (Applied Biosystems). PCR cocktails included either a single gene specific probe and primers set, or two multiplexed probe and primers sets (a set specific for the target clone and another gene-specific set multiplexed with the target probe). PCR reactions were set up using TaqMan® One-Step RT-PCR Master Mix (Applied Biosystems, Catalog No. 4313803) following manufacturer's instructions. Reverse transcription was performed at 48° C. for 30 minutes followed by amplification/PCR cycles as follows: 95° C. 10 min, then 40 cycles of 95° C. for 15 seconds, 60° C. for 1 minute. Results were recorded as CT values (cycle at which a given sample crosses a threshold level of fluorescence) using a log scale, with the difference in RNA concentration between a given sample and the sample with the lowest CT value being represented as 2 to the power of delta CT. The percent relative expression is then obtained by taking the reciprocal of this RNA difference and multiplying by 100.

When working with sscDNA samples, normalized sscDNA was used as described previously for RNA samples. PCR reactions containing one or two sets of probe and primers were set up as described previously, using 1×TaqMan® Universal Master mix (Applied Biosystems; catalog No. 4324020), following the manufacturer's instructions. PCR amplification was performed as follows: 95° C. 10 min, then 40 cycles of 95° C. for 15 seconds, 60° C. for 1 minute. Results were analyzed and processed as described previously.

Panels 1, 1.1, 1.2, and 1.3D

The plates for Panels 1, 1.1, 1.2 and 1.3D include 2 control wells (genomic DNA control and chemistry control) and 94 wells containing cDNA from various samples. The samples in these panels are broken into 2 classes: samples derived from cultured cell lines and samples derived from primary normal tissues. The cell lines are derived from cancers of the following types: lung cancer, breast cancer, melanoma, colon cancer, prostate cancer, CNS cancer, squamous cell carcinoma, ovarian cancer, liver cancer, renal cancer, gastric cancer and pancreatic cancer. Cell lines used in these panels are widely available through the American Type Culture Collection (ATCC), a repository for cultured cell lines, and were cultured using the conditions recommended by the ATCC. The normal tissues found on these panels are comprised of samples derived from all major organ systems from single adult individuals or fetuses. These samples are derived from the following organs: adult skeletal muscle, fetal skeletal muscle, adult heart, fetal heart, adult kidney, fetal kidney, adult liver, fetal liver, adult lung, fetal lung, various regions of the brain, the spleen, bone marrow, lymph node, pancreas, salivary gland, pituitary gland, adrenal gland, spinal cord, thymus, stomach, small intestine, colon, bladder, trachea, breast, ovary, uterus, placenta, prostate, testis and adipose.

In the results for Panels 1, 1.1, 1.2 and 1.3D, the following abbreviations are used:

ca.=carcinoma,

*=established from metastasis,

met=metastasis,

s cell var=small cell variant,

non-s=non-sm=non-small,

squam=squamous,

pl. eff=pl effusion=pleural effusion,

glio=glioma,

astro=astrocytoma, and

neuro=neuroblastoma.

General_screening_panel_v1.4

The plates for Panel 1.4 include 2 control wells (genomic DNA control and chemistry control) and 94 wells containing cDNA from various samples. The samples in Panel 1.4 are broken into 2 classes: samples derived from cultured cell lines and samples derived from primary normal tissues. The cell lines are derived from cancers of the following types: lung cancer, breast cancer, melanoma, colon cancer, prostate cancer, CNS cancer, squamous cell carcinoma, ovarian cancer, liver cancer, renal cancer, gastric cancer and pancreatic cancer. Cell lines used in Panel 1.4 are widely available through the American Type Culture Collection (ATCC), a repository for cultured cell lines, and were cultured using the conditions recommended by the ATCC. The normal tissues found on Panel 1.4 are comprised of pools of samples derived from all major organ systems from 2 to 5 different adult individuals or fetuses. These samples are derived from the following organs: adult skeletal muscle, fetal skeletal muscle, adult heart, fetal heart, adult kidney, fetal kidney, adult liver, fetal liver, adult lung, fetal lung, various regions of the brain, the spleen, bone marrow, lymph node, pancreas, salivary gland, pituitary gland, adrenal gland, spinal cord, thymus, stomach, small intestine, colon, bladder, trachea, breast, ovary, uterus, placenta, prostate, testis and adipose. Abbreviations are as described for Panels 1, 1.1, 1.2, and 1.3D.

Panels 2D and 2.2

The plates for Panels 2D and 2.2 generally include 2 control wells and 94 test samples composed of RNA or cDNA isolated from human tissue procured by surgeons working in close cooperation with the National Cancer Institute's Cooperative Human Tissue Network (CHTN) or the National Disease Research Initiative (NDRI). The tissues are derived from human malignancies and in cases where indicated many malignant tissues have “matched margins” obtained from noncancerous tissue just adjacent to the tumor. These are termed normal adjacent tissues and are denoted “NAT” in the results below. The tumor tissue and the “matched margins” are evaluated by two independent pathologists (the surgical pathologists and again by a pathologist at NDRI or CHTN). This analysis provides a gross histopathological assessment of tumor differentiation grade. Moreover, most samples include the original surgical pathology report that provides information regarding the clinical stage of the patient. These matched margins are taken from the tissue surrounding (i.e. immediately proximal) to the zone of surgery (designated “NAT”, for normal adjacent tissue, in Table RR). In addition, RNA and cDNA samples were obtained from various human tissues derived from autopsies performed on elderly people or sudden death victims (accidents, etc.). These tissues were ascertained to be free of disease and were purchased from various commercial sources such as Clontech (Palo Alto, Calif.), Research Genetics, and Invitrogen.

Panel 3D

The plates of Panel 3D are comprised of 94 cDNA samples and two control samples. Specifically, 92 of these samples are derived from cultured human cancer cell lines, 2 samples of human primary cerebellar tissue and 2 controls. The human cell lines are generally obtained from ATCC (American Type Culture Collection), NCI or the German tumor cell bank and fall into the following tissue groups: Squamous cell carcinoma of the tongue, breast cancer, prostate cancer, melanoma, epidermoid carcinoma, sarcomas, bladder carcinomas, pancreatic cancers, kidney cancers, leukemias/lymphomas, ovarian/uterine/cervical, gastric, colon, lung and CNS cancer cell lines. In addition, there are two independent samples of cerebellum. These cells are all cultured under standard recommended conditions and RNA extracted using the standard procedures. The cell lines in panel 3D and 1.3D are of the most common cell lines used in the scientific literature.

Panels 4D, 4R, and 4.1D

Panel 4 includes samples on a 96 well plate (2 control wells, 94 test samples) composed of RNA (Panel 4R) or cDNA (Panels 4D/4.1D) isolated from various human cell lines or tissues related to inflammatory conditions. Total RNA from control normal tissues such as colon and lung (Stratagene, La Jolla, Calif.) and thymus and kidney (Clontech) was employed. Total RNA from liver tissue from cirrhosis patients and kidney from lupus patients was obtained from BioChain (Biochain Institute, Inc., Hayward, Calif.). Intestinal tissue for RNA preparation from patients diagnosed as having Crohn's disease and ulcerative colitis was obtained from the National Disease Research Interchange (NDRI) (Philadelphia, Pa.).

Astrocytes, lung fibroblasts, dermal fibroblasts, coronary artery smooth muscle cells, small airway epithelium, bronchial epithelium, microvascular dermal endothelial cells, microvascular lung endothelial cells, human pulmonary aortic endothelial cells, human umbilical vein endothelial cells were all purchased from Clonetics (Walkersville, Md.) and grown in the media supplied for these cell types by Clonetics. These primary cell types were activated with various cytokines or combinations of cytokines for 6 and/or 12-14 hours, as indicated. The following cytokines were used; IL-1 beta at approximately 1-5 ng/ml, TNF alpha at approximately 5-10 ng/ml, IFN gamma at approximately 20-50 ng/ml, IL-4 at approximately 5-10 ng/ml, IL-9 at approximately 5-10 ng/ml, IL-13 at approximately 5-10 ng/ml. Endothelial cells were sometimes starved for various times by culture in the basal media from Clonetics with 0.1% serum.

Mononuclear cells were prepared from blood of employees at CuraGen Corporation, using Ficoll. LAK cells were prepared from these cells by culture in DMEM 5% FCS (Hyclone), 100 μM non essential amino acids (Gibco/Life Technologies, Rockville, Md.), 1 mM sodium pyruvate (Gibco), mercaptoethanol 5.5×10⁻⁵M (Gibco), and 10 mM Hepes (Gibco) and Interleukin 2 for 4-6 days. Cells were then either activated with 10-20 ng/ml PMA and 1-2 μg/ml ionomycin, IL-12 at 5-10 ng/ml, IFN gamma at 20-50 ng/ml and IL-18 at 5-10 g/ml for 6 hours. In some cases, mononuclear cells were cultured for 4-5 days in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco) with PHA (phytohemagglutinin) or PWM (pokeweed mitogen) at approximately 5 μg/ml. Samples were taken at 24, 48 and 72 hours for RNA preparation. MLR (mixed lymphocyte reaction) samples were obtained by taking blood from two donors, isolating the mononuclear cells using Ficoll and mixing the isolated mononuclear cells 1:1 at a final concentration of approximately 2×10⁶cells/ml in DMEM 5% FCS (Hyclone), 100 μM non essential amino acids (Gibco), 1 mM sodium pyruvate (Gibco), mercaptoethanol (5.5×10⁻⁵M) (Gibco), and 10 mM Hepes (Gibco). The MLR was cultured and samples taken at various time points ranging from 1-7 days for RNA preparation.

Monocytes were isolated from mononuclear cells using CD 14 Miltenyi Beads, +ve VS selection columns and a Vario Magnet according to the manufacturer's instructions. Monocytes were differentiated into dendritic cells by culture in DMEM 5% fetal calf serum (FCS) (Hyclone, Logan, Utah), 100 μM non essential amino acids (Gibco), 1 mM sodium pyruvate (Gibco), mercaptoethanol 5.5×10⁻⁵M (Gibco), and 10 mM Hepes (Gibco), 50 ng/ml GMCSF and 5 ng/ml IL-4 for 5-7 days. Macrophages were prepared by culture of monocytes for 5-7 days in DMEM 5% FCS (Hyclone), 100 μM non essential amino acids (Gibco), 1 mM sodium pyruvate (Gibco), mercaptoethanol 5.5×10⁻⁵M (Gibco), 10 mM Hepes (Gibco) and 10% AB Human Serum or MCSF at approximately 50 ng/ml. Monocytes, macrophages and dendritic cells were stimulated for 6 and 12-14 hours with lipopolysaccharide (LPS) at 100 ng/ml. Dendritic cells were also stimulated with anti-CD40 monoclonal antibody (Pharmingen) at 10 μg/ml for 6 and 12-14 hours.

CD4 lymphocytes, CD8 lymphocytes and NK cells were also isolated from mononuclear cells using CD4, CD8 and CD56 Miltenyi beads, positive VS selection columns and a Vario Magnet according to the manufacturer's instructions. CD45RA and CD45RO CD4 lymphocytes were isolated by depleting mononuclear cells of CD8, CD56, CD14 and CD19 cells using CD8, CD56, CD14 and CD19 Miltenyi beads and positive selection. CD45RO beads were then used to isolate the CD45RO CD4 lymphocytes with the remaining cells being CD45RA CD4 lymphocytes. CD45RA CD4, CD45RO CD4 and CD8 lymphocytes were placed in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco) and plated at 10⁶cells/ml onto Falcon 6 well tissue culture plates that had been coated overnight with 0.5 μg/ml anti-CD28 (Pharmingen) and 3 ug/ml anti-CD3 (OKT3, ATCC) in PBS. After 6 and 24 hours, the cells were harvested for RNA preparation. To prepare chronically activated CD8 lymphocytes, we activated the isolated CD8 lymphocytes for 4 days on anti-CD28 and anti-CD3 coated plates and then harvested the cells and expanded them in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco) and IL-2. The expanded CD8 cells were then activated again with plate bound anti-CD3 and anti-CD28 for 4 days and expanded as before. RNA was isolated 6 and 24 hours after the second activation and after 4 days of the second expansion culture. The isolated NK cells were cultured in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco) and IL-2 for 4-6 days before RNA was prepared.

To obtain B cells, tonsils were procured from NDRI. The tonsil was cut up with sterile dissecting scissors and then passed through a sieve. Tonsil cells were then spun down and resupended at 10⁶cells/ml in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco). To activate the cells, we used PWM at 5 μg/ml or anti-CD40 (Pharmingen) at approximately 10 μg/ml and IL-4 at 5-10 ng/ml. Cells were harvested for RNA preparation at 24,48 and 72 hours.

To prepare the primary and secondary Th1/Th2 and Tr1 cells, six-well Falcon plates were coated overnight with 10 μg/ml anti-CD28 (Pharmingen) and 2 μg/ml OKT3 (ATCC), and then washed twice with PBS. Umbilical cord blood CD4 lymphocytes (Poietic Systems, German Town, Md.) were cultured at 10⁵-10⁶cells/mi in DMEM 5% FCS (ilyclone), 100 μM non essential amino acids (Gibco), 1 mM sodium pyruvate (Gibco), mercaptoethanol 5.5×10⁻⁵M (Gibco), 10 mM Hepes (Gibco) and IL-2 (4 ng/ml). IL-12 (5 ng/ml) and anti-IL4 (1 μg/ml) were used to direct to Th1, while IL-4 (5 ng/ml) and anti-IFN gamma (1 μg/ml) were used to direct to Th2 and IL-10 at 5 ng/ml was used to direct to Tr1. After 4-5 days, the activated Th1, Th2 and Tr1 lymphocytes were washed once in DMEM and expanded for 4-7 days in DMEM 5% FCS (Hyclone), 100 μM non essential amino acids (Gibco), 1 mM sodium pyruvate (Gibco), mercaptoethanol 5.5×10⁻⁵M (Gibco), 10 mM Hepes (Gibco) and IL-2 (1 ng/ml). Following this, the activated Th1, Th2 and Tr1 lymphocytes were re-stimulated for 5 days with anti-CD28/OKT3 and cytokines as described above, but with the addition of anti-CD95L (1 μg/ml) to prevent apoptosis. After 4-5 days, the Th1, Th2 and Tr1 lymphocytes were washed and then expanded again with IL-2 for 4-7 days. Activated Th1 and Th2 lymphocytes were maintained in this way for a maximum of three cycles. RNA was prepared from primary and secondary Th1, Th2 and Tr1 after 6 and 24 hours following the second and third activations with plate bound anti-CD3 and anti-CD28 mAbs and 4 days into the second and third expansion cultures in Interleukin 2.

The following leukocyte cells lines were obtained from the ATCC: Ramos, EOL-1, KU-812. EOL cells were further differentiated by culture in 0.1 mM dbcAMP at 5×10⁵ cells/ml for 8 days, changing the media every 3 days and adjusting the cell concentration to 5×10⁵ cells/ml. For the culture of these cells, we used DMEM or RPMI (as recommended by the ATCC), with the addition of 5% FCS (Hyclone), 100 μM non essential amino acids (Gibco), 1 mM sodium pyruvate (Gibco), mercaptoethanol 5.5×10⁻⁵M (Gibco), 10 mM Hepes (Gibco). RNA was either prepared from resting cells or cells activated with PMA at 10 ng/ml and ionomycin at 1 μg/ml for 6 and 14 hours. Keratinocyte line CCD106 and an airway epithelial tumor line NCI-H292 were also obtained from the ATCC. Both were cultured in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco). CCD1106 cells were activated for 6 and 14 hours with approximately 5 ng/ml TNF alpha and 1 ng/ml IL-1 beta, while NCI-H292 cells were activated for 6 and 14 hours with the following cytokines: 5 ng/ml IL-4, 5 ng/ml IL-9, 5 ng/ml IL-13 and 25 ng/ml IFN gamma.

For these cell lines and blood cells, RNA was prepared by lysing approximately 10⁷ cells/ml using Trizol (Gibco BRL). Briefly, 1/10 volume of bromochloropropane (Molecular Research Corporation) was added to the RNA sample, vortexed and after 10 minutes at room temperature, the tubes were spun at 14,000 rpm in a Sorvall SS34 rotor. The aqueous phase was removed and placed in a 15 ml Falcon Tube. An equal volume of isopropanol was added and left at −20° C. overnight. The precipitated RNA was spun down at 9,000 rpm for 15 min in a Sorvall SS34 rotor and washed in 70% ethanol. The pellet was redissolved in 300 μl of RNAse-free water and 35 μl buffer (Promega) 5 μl DTT, ⁷ μl RNAsin and ⁸ μl DNAse were added. The tube was incubated at 37° C. for 30 minutes to remove contaminating genomic DNA, extracted once with phenol chloroform and re-precipitated with 1/10 volume of 3M sodium acetate and 2 volumes of 100% ethanol. The RNA was spun down and placed in RNAse free water. RNA was stored at −80° C.

AI_Comprehensive Panel_v1.0

The plates for AI_comprehensive panel_v1.0 include two control wells and 89 test samples comprised of cDNA isolated from surgical and postmortem human tissues obtained, from the Backus Hospital and Clinomics (Frederick, Md.). Total RNA was extracted from tissue samples from the Backus Hospital in the Facility at CuraGen. Total RNA from other tissues was obtained from Clinomics.

Joint tissues including synovial fluid, synovium, bone and cartilage were obtained from patients undergoing total knee or hip replacement surgery at the Backus Hospital. Tissue samples were immediately snap frozen in liquid nitrogen to ensure that isolated RNA was of optimal quality and not degraded. Additional samples of osteoarthritis and rheumatoid arthritis joint tissues were obtained from Clinomics. Normal control tissues were supplied by Clinomics and were obtained during autopsy of trauma victims.

Surgical specimens of psoriatic tissues and adjacent matched tissues were provided as total RNA by Clinomics. Two male and two female patients were selected between the ages of 25 and 47. None of the patients were taking prescription drugs at the time samples were isolated.

Surgical specimens of diseased colon from patients with ulcerative colitis and Crohns disease and adjacent matched tissues were obtained from Clinomics. Bowel tissue from three female and three male Crohn's patients between the ages of 41-69 were used. Two patients were not on prescription medication while the others were taking dexamethasone, phenobarbital, or tylenol. Ulcerative colitis tissue was from three male and four female patients. Four of the patients were taking lebvid and two were on phenobarbital.

Total RNA from post mortem lung tissue from trauma victims with no disease or with emphysema, asthma or COPD was purchased from Clinomics. Emphysema patients ranged in age from 40-70 and all were smokers, this age range was chosen to focus on patients with cigarette-linked emphysema and to avoid those patients with alpha-1anti-trypsin deficiencies. Asthma patients ranged in age from 36-75, and excluded smokers to prevent those patients that could also have COPD. COPD patients ranged in age from 35-80 and included both smokers and non-smokers. Most patients were taking corticosteroids, and bronchodilators.

In the labels employed to identify tissues in the AI_comprehensive panel_v1.0 panel, the following abbreviations are used:

AI=Autoimmunity

Syn=Synovial

Normal =No apparent disease

Rep22 /Rep20=individual patients

RA=Rheumatoid arthritis

Backus=From Backus Hospital

OA=Osteoarthritis

(SS) (BA) (MF)=Individual patients

Adj=Adjacent tissue

Match control=adjacent tissues

-M=Male

-F=Female

COPD=Chronic obstructive pulmonary disease

Panels 5D and 51

The plates for Panel 5D and 5I include two control wells and a variety of cDNAs isolated from human tissues and cell lines with an emphasis on metabolic diseases. Metabolic tissues were obtained from patients enrolled in the Gestational Diabetes study. Cells were obtained during different stages in the differentiation of adipocytes from human mesenchymal stem cells. Human pancreatic islets were also obtained.

In the Gestational Diabetes study subjects are young (18-40 years), otherwise healthy women with and without gestational diabetes undergoing routine (elective) Caesarean section. After delivery of the infant, when the surgical incisions were being repaired/closed, the obstetrician removed a small sample.

Patient 2: Diabetic Hispanic, overweight, not on insulin

Patient 7-9: Nondiabetic Caucasian and obese (BMI>30)

Patient 10: Diabetic Hispanic, overweight, on insulin

Patient 11: Nondiabetic African American and overweight

Patient 12: Diabetic Hispanic on insulin

Adipocyte differentiation was induced in donor progenitor cells obtained from Osirus (a division of Clonetics/BioWhittaker) in triplicate, except for Donor 3U which had only two replicates. Scientists at Clonetics isolated, grew and differentiated human mesenchymal stem cells (HuMSCs) for CuraGen based on the published protocol found in Mark F. Pittenger, et al., Multilineage Potential of Adult Human Mesenchymal Stem Cells Science Apr. 2 1999: 143-147. Clonetics provided Trizol lysates or frozen pellets suitable for mRNA isolation and ds cDNA production. A general description of each donor is as follows:

Donor 2 and 3 U: Mesenchymal Stem cells, Undifferentiated Adipose

Donor 2 and 3 AM: Adipose, AdiposeMidway Differentiated

Donor 2 and 3 AD: Adipose, Adipose Differentiated

Human cell lines were generally obtained from ATCC (American Type Culture Collection), NCI or the German tumor cell bank and fall into the following tissue groups: kidney proximal convoluted tubule, uterine smooth muscle cells, small intestine, liver HepG2 cancer cells, heart primary stromal cells, and adrenal cortical adenoma cells. These cells are all cultured under standard recommended conditions and RNA extracted using the standard procedures. All samples were processed at CuraGen to produce single stranded cDNA.

Panel 5I contains all samples previously described with the addition of pancreatic islets from a 58 year old female patient obtained from the Diabetes Research Institute at the University of Miami School of Medicine. Islet tissue was processed to total RNA at an outside source and delivered to CuraGen for addition to panel 5I.

In the labels employed to identify tissues in the 5D and 5I panels, the following abbreviations are used:

GO Adipose=Greater Omentum Adipose

SK=Skeletal Muscle

UT=Uterus

PL=Placenta

AD=Adipose Differentiated

AM=Adipose Midway Differentiated

U=Undifferentiated Stem Cells

Panel CNSD.01

The plates for Panel CNSD.01 include two control wells and 94 test samples comprised of cDNA isolated from postmortem human brain tissue obtained from the Harvard Brain Tissue Resource Center. Brains are removed from calvaria of donors between 4 and 24 hours after death, sectioned by neuroanatomists, and frozen at −80° C. in liquid nitrogen vapor. All brains are sectioned and examined by neuropathologists to confirm diagnoses with clear associated neuropathology.

Disease diagnoses are taken from patient records. The panel contains two brains from each of the following diagnoses: Alzheimer's disease, Parkinson's disease, Huntington's disease, Progressive Supernuclear Palsy, Depression, and “Normal controls”. Within each of these brains, the following regions are represented: cingulate gyrus, temporal pole, globus palladus, substantia nigra, Brodman Area 4 (primary motor strip), Brodman Area 7 (parietal cortex), Brodman Area 9 (prefrontal cortex), and Brodman area 17 (occipital cortex). Not all rain regions are represented in all cases; e.g., Huntington's disease is characterized in part by neurodegeneration in the globus palladus, thus this region is impossible to obtain from confirmed Huntington's cases. Likewise Parkinson's disease is characterized by degeneration of the substantia nigra making this region more difficult to obtain. Normal control brains were examined for neuropathology and found to be free of any pathology consistent with neurodegeneration.

In the labels employed to identify tissues in the CNS panel, the following abbreviations are used:

PSP=Progressive supranuclear palsy

Sub Nigra=Substantia nigra

Glob Palladus=Globus palladus

Temp Pole=Temporal pole

Cing Gyr=Cingulate gyrus

BA 4=Brodman Area 4

Panel CNS_Neurodegeneration_V1.0

The plates for Panel CNS_Neurodegeneration_V1.0 include two control wells and 47 test samples comprised of cDNA isolated from postmortem human brain tissue obtained from the Harvard Brain Tissue Resource Center (McLean Hospital) and the Human Brain and Spinal Fluid Resource Center (VA Greater Los Angeles Healthcare System). Brains are removed from calvaria of donors between 4 and 24 hours after death, sectioned by neuroanatomists, and frozen at −80° C. in liquid nitrogen vapor. All brains are sectioned and examined by neuropathologists to confirm diagnoses with clear associated neuropathology.

Disease diagnoses are taken from patient records. The panel contains six brains from Alzheimer's disease (AD) patients, and eight brains from “Normal controls” who showed no evidence of dementia prior to death. The eight normal control brains are divided into two categories: Controls with no dementia and no Alzheimer's like pathology (Controls) and controls with no dementia but evidence of severe Alzheimer's like pathology, (specifically senile plaque load rated as level 3 on a scale of 0-3; 0=no evidence of plaques, 3=severe AD senile plaque load). Within each of these brains, the following regions are represented: hippocampus, temporal cortex (Brodman Area 21), parietal cortex (Brodman area 7), and occipital cortex (Brodman area 17). These regions were chosen to encompass all levels of neurodegeneration in AD. The hippocampus is a region of early and severe neuronal loss in AD; the temporal cortex is known to show neurodegeneration in AD after the hippocampus; the parietal cortex shows moderate neuronal death in the late stages of the disease; the occipital cortex is spared in AD and therefore acts as a “control” region within AD patients. Not all brain regions are represented in all cases.

In the labels employed to identify tissues in the CNS_Neurodegeneration_V1.0 panel, the following abbreviations are used:

AD=Alzheimer's disease brain; patient was demented and showed AD-like pathology upon autopsy

Control=Control brains; patient not demented, showing no neuropathology

Control (Path)=Control brains; pateint not demented but showing sever AD-like pathology

SupTemporal Ctx=Superior Temporal Cortex

Inf Temporal Ctx=Inferior Temporal Cortex

NOV1b, NOV1c

Expression of NOV1b and NOV1c was assessed using the primer-probe sets Ag1848, Ag2263, Ag2422 and Ag1522, described in Tables 14, 15, 16 and 17. Results of the RTQ-PCR runs are shown in Tables 18, 19, 20, 21, 22, 23 and 24.

TABLE 14

Probe Name Ag1848

| | | | | |
|---|---|---|---|---|
| | | | Start | |
| Primers | Sequences | Length | Position | SEQ ID NO: |

| | | | | |
|---|---|---|---|---|
| Forward | 5′-TGACTTCGACACAGACATCACT-3′ | 22 | 1234 | 133 |
| Probe | TET-5′-ACTCATCTGCTGCCCTGACTGGTG-3′- | 24 | 1257 | 134 |
| | TAMRA | | | |
| Reverse | 5′-CCTTGCCGTCTTAAAGTTGAC-3′ | 21 | 1292 | 135 |

TABLE 15

Probe Name Ag2263

| | | | | |
|---|---|---|---|---|
| | | | Start | |
| Primers | Sequences | Length | Position | SEQ ID NO: |

| | | | | |
|---|---|---|---|---|
| Forward | 5′-TGACTTCGACACAGACATCACT-3′ | 22 | 1234 | 136 |
| Probe | TET-5′-ACTCATCTGCTGCCCTGACTGGTG-3′- | 24 | 1257 | 137 |
| | TAMRA | | | |
| Reverse | 5′-CCTTGCCGTCTTAAAGTTGAC-3′ | 21 | 1292 | 138 |

TABLE 16

Probe Name Ag2422

| | | | | |
|---|---|---|---|---|
| | | | Start | |
| Primers | Sequences | Length | Position | SEQ ID NO: |

| | | | | |
|---|---|---|---|---|
| Forward | 5′-GGCTCCCTGGACACTCTCT-3′ | 19 | 2522 | 139 |
| Probe | TET-5′-CTGTCACCACCCAGCTGCGACCTTAT-3′- | 26 | 2559 | 140 |
| | TAMRA | | | |
| Reverse | 5′-TGGACAGTGGGATCTTGAAG-3′ | 20 | 2587 | 141 |

TABLE 17

Probe Name Ag1522

| | | | | |
|---|---|---|---|---|
| | | | Start | |
| Primers | Sequences | Length | Position | SEQ ID NO: |

| | | | | |
|---|---|---|---|---|
| Forward | 5′-TGACTTCGACACAGACATCACT-3′ | 22 | 1234 | 142 |
| Probe | TET-5′-ACTCATCTGCTGCCCTGACTGGTG-3′- | 24 | 1257 | 143 |
| | TAMRA | | | |
| Reverse | 5′-CCTTGCCGTCTTAAAGTTGAC-3′ | 21 | 1292 | 144 |

TABLE 18
CNS_neurodegeneration_v1.0

| | Rel. | Rel. | Rel. | Rel. | Rel. |
|---|---|---|---|---|---|
| | Exp. (%) | Exp. (%) | Exp. (%) | Exp. (%) | Exp. (%) |
| | Ag1848, | Ag2263, | Ag2263, | Ag2422, | Ag2422, |
| | Run | Run | Run | Run | Run |
| Tissue Name | 207776125 | 219933384 | 224115886 | 206262709 | 230512499 |
| AD 1 Hippo | 28.3 | 39.0 | 19.3 | 21.3 | 16.6 |
| AD 2 Hippo | 37.9 | 45.1 | 23.5 | 38.7 | 40.1 |
| AD 3 Hippo | 12.0 | 20.6 | 13.9 | 14.9 | 13.0 |
| AD 4 Hippo | 17.7 | 27.2 | 9.0 | 13.3 | 16.4 |
| AD 5 Hippo | 45.4 | 60.3 | 8.1 | 57.8 | 59.0 |
| AD 6 Hippo | 66.9 | 96.6 | 70.2 | 95.9 | 66.0 |
| Control 2 Hippo | 43.2 | 81.2 | 67.8 | 46.0 | 48.3 |
| Control 4 Hippo | 34.2 | 36.6 | 38.7 | 30.4 | 27.5 |
| Control (Path) 3 Hippo | 3.9 | 11.0 | 4.6 | 12.7 | 12.1 |
| AD 1 Temporal Ctx | 47.0 | 79.0 | 69.7 | 40.6 | 27.2 |
| AD 2 Temporal Ctx | 49.3 | 61.6 | 70.7 | 39.8 | 50.7 |
| AD 3 Temporal Ctx | 14.5 | 20.7 | 15.3 | 15.7 | 14.5 |
| AD 4 Temporal Ctx | 41.5 | 53.6 | 31.9 | 36.3 | 39.0 |
| AD 5 Inf Temporal Ctx | 77.9 | 95.9 | 72.2 | 88.9 | 100.0 |
| AD 5 Sup Temporal Ctx | 40.9 | 57.4 | 3.7 | 57.0 | 69.3 |
| AD 6 Inf Temporal Ctx | 84.1 | 99.3 | 100.0 | 74.2 | 83.5 |
| AD 6 Sup Temporal Ctx | 58.2 | 64.6 | 81.8 | 71.7 | 61.1 |
| Control 1 Temporal Ctx | 17.9 | 18.0 | 21.5 | 11.3 | 16.5 |
| Control 2 Temporal Ctx | 45.7 | 39.8 | 66.4 | 44.8 | 55.1 |
| Control 3 Temporal Ctx | 14.7 | 21.8 | 22.7 | 15.6 | 13.5 |
| Control 3 Temporal Ctx | 23.2 | 21.5 | 23.8 | 19.1 | 24.1 |
| Control (Path) 1 Temporal | 46.0 | 39.8 | 19.3 | 40.3 | 51.1 |
| Ctx | | | | | |
| Control (Path) 2 Temporal | 24.7 | 40.6 | 23.7 | 21.8 | 24.0 |
| Ctx | | | | | |
| Control (Path) 3 Temporal | 6.0 | 8.2 | 8.0 | 7.7 | 7.3 |
| Ctx | | | | | |
| Control (Path) 4 Temporal | 32.1 | 29.5 | 31.0 | 24.0 | 18.6 |
| Ctx | | | | | |
| AD 1 Occipital Ctx | 24.1 | 48.0 | 5.5 | 26.4 | 13.7 |
| AD 2 Occipital Ctx | 0.0 | 0.0 | 0.0 | 0.0 | 0.0 |
| (Missing) | | | | | |
| AD 3 Occipital Ctx | 19.2 | 25.3 | 20.4 | 18.2 | 18.8 |
| AD 4 Occipital Ctx | 30.1 | 58.2 | 30.6 | 23.3 | 30.8 |
| AD 5 Occipital Ctx | 6.0 | 39.0 | 8.5 | 26.8 | 23.0 |
| AD 5 Occipital Ctx | 43.2 | 51.8 | 53.6 | 50.3 | 47.6 |
| Control 1 Occipital Ctx | 14.6 | 22.2 | 19.1 | 12.8 | 13.4 |
| Control 2 Occipital Ctx | 66.9 | 85.9 | 94.6 | 76.3 | 70.2 |
| Control 3 Occipital Ctx | 17.8 | 37.1 | 8.0 | 17.4 | 13.1 |
| Control 4 Occipital Ctx | 23.3 | 22.2 | 2.7 | 15.7 | 19.1 |
| Control (Path) 1 Occipital | 100.0 | 100.0 | 63.7 | 100.0 | 90.1 |
| Ctx | | | | | |
| Control (Path) 2 Occipital | 18.7 | 20.9 | 11.0 | 12.3 | 11.7 |
| Ctx | | | | | |
| Control (Path) 3 Occipital | 7.9 | 6.1 | 9.4 | 7.1 | 5.8 |
| Ctx | | | | | |
| Control (Path) 4 Occipital | 24.5 | 21.5 | 11.1 | 14.0 | 13.1 |
| Ctx | | | | | |
| Control 1 Parietal Ctx | 23.2 | 26.8 | 7.4 | 22.2 | 17.6 |
| Control 2 Parietal Ctx | 46.0 | 65.1 | 71.2 | 64.6 | 50.0 |
| Control 3 Parietal Ctx | 26.1 | 27.2 | 16.5 | 17.3 | 19.5 |
| Control (Path) 1 Parietal | 51.1 | 66.0 | 80.1 | 54.3 | 55.1 |
| Ctx | | | | | |
| Control (Path) 2 Parietal | 36.3 | 16.5 | 34.2 | 27.9 | 27.9 |
| Ctx | | | | | |
| Control (Path) 3 Parietal | 6.1 | 10.5 | 1.4 | 5.1 | 4.6 |
| Ctx | | | | | |
| Control (Path) 4 Parietal | 46.0 | 52.5 | 10.7 | 36.6 | 12.2 |
| Ctx | | | | | |

TABLE 19
Panel 1.2

| | Rel. Exp. (%) | | Rel. Exp. (%) |
|---|---|---|---|
| | Ag1522, Run | | Ag1522, Run |
| Tissue Name | 142131145 | Tissue Name | 142131145 |
| Endothelial cells | 1.2 | Renal ca. 786-0 | 0.0 |
| Heart (Fetal) | 17.9 | Renal ca. A498 | 0.3 |
| Pancreas | 0.7 | Renal ca. RXF 393 | 0.2 |
| Pancreatic ca. CAPAN 2 | 4.9 | Renal ca. ACHN | 0.1 |
| Adrenal Gland | 7.9 | Renal ca. UO-31 | 0.5 |
| Thyroid | 0.1 | Renal ca. TK-10 | 0.3 |
| Salivary gland | 2.5 | Liver | 2.4 |
| Pituitary gland | 0.1 | Liver (fetal) | 0.5 |
| Brain (fetal) | 0.2 | Liver ca. (hepatoblast) | 0.3 |
| | | HepG2 | |
| Brain (whole) | 3.2 | Lung | 0.3 |
| Brain (amygdala) | 4.4 | Lung (fetal) | 0.4 |
| Brain (cerebellum) | 9.0 | Lung ca. (small cell) LX-1 | 25.3 |
| Brain (hippocampus) | 18.9 | Lung ca. (small cell) NCI- | 43.8 |
| | | H69 | |
| Brain (thalamus) | 15.7 | Lung ca. (s.cell var.) SHP- | 0.3 |
| | | 77 | |
| Cerebral Cortex | 35.4 | Lung ca. (large cell)NCI- | 54.7 |
| | | H460 | |
| Spinal cord | 1.6 | Lung ca. (non-sm. cell) | 0.3 |
| | | A549 | |
| glio/astro U87-MG | 72.2 | Lung ca. (non-s.cell) NCI- | 2.4 |
| | | H23 | |
| glio/astro U-118-MG | 3.1 | Lung ca. (non-s.cell) HOP- | 1.7 |
| | | 62 | |
| astrocytoma SW1783 | 0.3 | Lung ca. (non-s.cl) NCI- | 9.3 |
| | | H522 | |
| neuro*; met SK-N-AS | 36.3 | Lung ca. (squam.) SW 900 | 1.5 |
| astrocytoma SF-539 | 5.8 | Lung ca. (squam.) NCI- | 22.4 |
| | | H596 | |
| astrocytoma SNB-75 | 1.7 | Mammary gland | 1.4 |
| glioma SNB-19 | 23.8 | Breast ca.* (pl.ef) MCF-7 | 0.8 |
| glioma U251 | 2.9 | Breast ca.* (pl.ef) MDA- | 0.1 |
| | | MB-231 | |
| glioma SF-295 | 100.0 | Breast ca.* (pl.ef) T47D | 18.4 |
| Heart | 31.6 | Breast ca. BT-549 | 0.1 |
| Skeletal Muscle | 3.4 | Breast ca. MDA-N | 0.0 |
| Bone marrow | 0.2 | Ovary | 6.9 |
| Thymus | 0.2 | Ovarian ca. OVCAR-3 | 1.7 |
| Spleen | 2.1 | Ovarian ca. OVCAR-4 | 12.9 |
| Lymph node | 0.5 | Ovarian ca. OVCAR-5 | 5.7 |
| Colorectal | 1.4 | Ovarian ca. OVCAR-8 | 5.3 |
| Stomach | 1.3 | Ovarian ca. IGROV-1 | 0.8 |
| Small intestine | 3.3 | Ovarian ca. (ascites) SK- | 5.4 |
| | | OV-3 | |
| Colon ca. SW480 | 0.8 | Uterus | 0.9 |
| Colon ca.* SW620 (SW480 | 2.2 | Placenta | 0.9 |
| met) | | | |
| Colon ca. HT29 | 0.1 | Prostate | 10.0 |
| Colon ca. HCT-116 | 7.5 | Prostate ca.* (bone met) | 0.1 |
| | | PC-3 | |
| Colon ca. CaCo-2 | 6.3 | Testis | 0.3 |
| CC Well to Mod Diff | 3.0 | Melanoma Hs688(A).T | 21.2 |
| (ODO3866) | | | |
| Colon ca. HCC-2998 | 1.2 | Melanoma* (met) | 28.5 |
| | | Hs688(B).T | |
| Gastric ca. (liver met) NCI- | 24.7 | Melanoma UACC-62 | 2.4 |
| N87 | | | |
| Bladder | 12.8 | Melanoma M14 | 0.1 |
| Trachea | 0.3 | Melanoma LOX IMVI | 0.1 |
| Kidney | 19.2 | Melanoma* (met) SK- | 1.2 |
| | | MEL-5 | |
| Kidney (fetal) | 6.6 | | |

TABLE 20
Panel 1.3D

| | Rel. Exp. (%) | Rel. Exp. (%) | Rel. Exp. (%) | Rel. Exp. (%) |
|---|---|---|---|---|
| | Ag1522, Run | Ag1848, Run | Ag2263, Run | Ag2422, Run |
| Tissue Name | 159601761 | 160201402 | 166011650 | 159319549 |
| Liver adenocarcinoma | 15.8 | 12.3 | 31.4 | 18.3 |
| Pancreas | 1.7 | 1.4 | 2.8 | 2.9 |
| Pancreatic ca. CAPAN 2 | 6.7 | 4.6 | 21.6 | 5.5 |
| Adrenal gland | 3.9 | 2.0 | 3.5 | 3.0 |
| Thyroid | 1.7 | 1.5 | 0.0 | 2.5 |
| Salivary gland | 0.6 | 0.2 | 2.3 | 0.3 |
| Pituitary gland | 2.1 | 1.4 | 2.9 | 4.3 |
| Brain (fetal) | 1.4 | 1.1 | 3.5 | 1.1 |
| Brain (whole) | 28.7 | 13.5 | 43.2 | 10.4 |
| Brain (amygdala) | 16.8 | 13.0 | 31.2 | 18.6 |
| Brain (cerebellum) | 8.2 | 6.5 | 42.3 | 9.2 |
| Brain (hippocampus) | 60.7 | 47.6 | 16.8 | 51.8 |
| Brain (substantia nigra) | 8.9 | 5.2 | 32.3 | 6.8 |
| Brain (thalamus) | 40.1 | 22.2 | 62.0 | 19.8 |
| Cerebral Cortex | 25.9 | 18.4 | 36.6 | 14.3 |
| Spinal cord | 10.2 | 5.4 | 37.9 | 7.9 |
| glio/astro U87-MG | 43.2 | 34.6 | 100.0 | 48.6 |
| glio/astro U-118-MG | 10.2 | 8.0 | 6.4 | 7.5 |
| astrocytoma SW1783 | 0.9 | 0.8 | 2.8 | 1.1 |
| neuro*; met SK-N-AS | 100.0 | 100.0 | 59.0 | 100.0 |
| astrocytoma SF-539 | 9.7 | 8.3 | 17.7 | 9.0 |
| astrocytoma SNB-75 | 12.9 | 12.1 | 8.4 | 12.1 |
| glioma SNB-19 | 19.5 | 17.6 | 46.3 | 17.2 |
| glioma U251 | 13.4 | 10.6 | 24.5 | 10.9 |
| glioma SF-295 | 66.9 | 62.4 | 64.2 | 62.0 |
| Heart (Fetal) | 15.6 | 12.5 | 20.0 | 18.7 |
| Heart | 2.2 | 1.1 | 3.4 | 3.3 |
| Skeletal muscle (Fetal) | 22.2 | 14.0 | 6.7 | 19.3 |
| Skeletal muscle | 0.3 | 0.2 | 1.4 | 0.7 |
| Bone marrow | 0.7 | 0.3 | 0.4 | 0.8 |
| Thymus | 2.0 | 1.6 | 3.6 | 3.4 |
| Spleen | 7.9 | 5.6 | 4.5 | 5.9 |
| Lymph node | 2.6 | 1.9 | 2.7 | 2.1 |
| Colorectal | 4.7 | 9.2 | 12.8 | 10.3 |
| Stomach | 6.1 | 2.4 | 3.6 | 4.5 |
| Small intestine | 2.9 | 2.9 | 4.5 | 4.9 |
| Colon ca. SW480 | 2.0 | 1.0 | 1.9 | 1.5 |
| Colon ca.* SW620 (SW480 met) | 1.0 | 1.2 | 2.0 | 2.1 |
| Colon ca. HT29 | 0.1 | 0.1 | 0.0 | 0.1 |
| Colon ca. HCT-116 | 4.2 | 2.9 | 4.7 | 5.6 |
| Colon ca. CaCo-2 | 5.3 | 3.9 | 12.5 | 7.2 |
| CC Well to Mod Diff | 14.8 | 17.3 | 19.8 | 23.5 |
| (ODO3866) | | | | |
| Colon ca. HCC-2998 | 0.7 | 1.6 | 0.0 | 0.5 |
| Gastric ca. (liver met) NCI-N87 | 21.9 | 22.8 | 19.1 | 25.7 |
| Bladder | 2.1 | 1.7 | 3.4 | 1.5 |
| Trachea | 12.2 | 6.8 | 1.6 | 13.8 |
| Kidney | 1.4 | 0.6 | 3.9 | 3.0 |
| Kidney (fetal) | 5.3 | 5.8 | 5.2 | 6.3 |
| Renal ca. 786-0 | 0.1 | 0.0 | 0.0 | 0.0 |
| Renal ca. A498 | 7.7 | 7.9 | 6.8 | 9.7 |
| Renal ca. RXF 393 | 0.1 | 3.6 | 0.8 | 0.1 |
| Renal ca. ACHN | 0.0 | 0.0 | 0.0 | 0.0 |
| Renal ca. UO-31 | 0.2 | 0.3 | 0.5 | 0.3 |
| Renal ca. TK-10 | 0.1 | 0.0 | 0.0 | 0.0 |
| Liver | 0.3 | 0.1 | 0.0 | 0.6 |
| Liver (fetal) | 1.1 | 1.0 | 0.3 | 1.2 |
| Liver ca. (hepatoblast) HepG2 | 0.2 | 0.0 | 0.8 | 0.3 |
| Lung | 8.2 | 9.4 | 4.1 | 10.3 |
| Lung (fetal) | 4.3 | 4.2 | 7.3 | 4.5 |
| Lung ca. (small cell) LX-1 | 8.4 | 6.9 | 31.6 | 9.9 |
| Lung ca. (small cell) NCI-H69 | 44.4 | 48.6 | 90.8 | 54.3 |
| Lung ca. (s.cell var.) SHP-77 | 0.7 | 0.8 | 0.5 | 1.1 |
| Lung ca. (large cell)NCI-H460 | 16.2 | 11.9 | 22.4 | 12.1 |
| Lung ca. (non-sm. cell) A549 | 0.4 | 0.3 | 0.2 | 0.4 |
| Lung ca. (non-s.cell) NCI-H23 | 2.0 | 0.9 | 3.3 | 1.2 |
| Lung ca. (non-s.cell) HOP-62 | 0.4 | 0.9 | 1.6 | 0.7 |
| Lung ca. (non-s.cl) NCI-H522 | 1.7 | 0.8 | 1.7 | 1.1 |
| Lung ca. (squam.) SW 900 | 0.5 | 0.3 | 1.9 | 0.2 |
| Lung ca. (squam.) NCI-H596 | 4.0 | 4.1 | 26.4 | 2.4 |
| Mammary gland | 6.3 | 4.4 | 3.0 | 2.8 |
| Breast ca.* (pl.ef) MCF-7 | 1.1 | 0.4 | 1.5 | 0.9 |
| Breast ca.* (pl.ef) MDA-MB- | 0.8 | 1.2 | 0.7 | 1.4 |
| 231 | | | | |
| Breast ca.* (pl. ef) T47D | 9.6 | 5.7 | 14.0 | 4.5 |
| Breast ca. BT-549 | 0.2 | 0.3 | 0.2 | 0.3 |
| Breast ca. MDA-N | 0.0 | 0.0 | 0.0 | 0.0 |
| Ovary | 6.4 | 4.9 | 6.2 | 9.5 |
| Ovarian ca. OVCAR-3 | 1.1 | 0.6 | 1.1 | 0.8 |
| Ovarian ca. OVCAR-4 | 1.0 | 1.4 | 11.4 | 1.5 |
| Ovarian ca. OVCAR-5 | 2.4 | 2.6 | 5.7 | 3.3 |
| Ovarian ca. OVCAR-8 | 3.6 | 1.6 | 2.6 | 5.4 |
| Ovarian ca. IGROV-1 | 0.6 | 0.2 | 0.7 | 0.2 |
| Ovarian ca. (ascites) SK-OV-3 | 2.0 | 2.6 | 2.1 | 1.1 |
| Uterus | 2.7 | 1.3 | 3.9 | 4.2 |
| Placenta | 2.0 | 2.0 | 5.8 | 4.8 |
| Prostate | 4.4 | 2.5 | 3.4 | 5.4 |
| Prostate ca.* (bone met) PC-3 | 0.1 | 0.1 | 0.2 | 0.0 |
| Testis | 8.1 | 5.5 | 3.5 | 6.4 |
| Melanoma Hs688(A).T | 31.6 | 25.0 | 59.5 | 27.4 |
| Melanoma* (met) Hs688(B).T | 46.0 | 17.1 | 87.1 | 28.5 |
| Melanoma UACC-62 | 0.1 | 0.2 | 2.0 | 0.5 |
| Melanoma M14 | 0.0 | 0.0 | 0.0 | 0.0 |
| Melanoma LOX IMVI | 0.1 | 0.2 | 0.0 | 0.1 |
| Melanoma* (met) SK-MEL-5 | 0.9 | 0.9 | 1.7 | 0.6 |
| Adipose | 3.6 | 2.3 | 5.1 | 2.9 |

TABLE 21
Panel 2D

| | Rel. Exp. (%) | Rel. Exp. (%) | Rel. Exp. (%) | Rel. Exp. (%) | Rel. Exp. (%) |
|---|---|---|---|---|---|
| | Ag1522, Run | Ag1522, Run | Ag1848, Run | Ag2263, Run | Ag2422, Run |
| Tissue Name | 145049854 | 145492337 | 160202834 | 165725935 | 159317774 |
| Normal Colon | 20.2 | 46.0 | 35.1 | 59.0 | 36.9 |
| CC Well to Mod | 15.3 | 45.1 | 22.5 | 21.8 | 21.3 |
| Diff (ODO3866) | | | | | |
| CC Margin | 6.1 | 15.2 | 7.4 | 7.7 | 5.5 |
| (ODO3866) | | | | | |
| CC Gr.2 | 7.0 | 8.2 | 5.8 | 5.9 | 13.2 |
| rectosigmoid | | | | | |
| (ODO3868) | | | | | |
| CC Margin | 0.3 | 0.5 | 0.5 | 9.3 | 0.8 |
| (ODO3868) | | | | | |
| CC Mod Diff | 1.2 | 4.0 | 2.5 | 5.6 | 5.8 |
| (ODO3920) | | | | | |
| CC Margin | 3.0 | 4.7 | 4.1 | 5.4 | 7.2 |
| (ODO3920) | | | | | |
| CC Gr.2 ascend | 10.7 | 22.5 | 24.1 | 19.9 | 25.5 |
| colon | | | | | |
| (ODO3921) | | | | | |
| CC Margin | 3.6 | 4.3 | 7.3 | 5.6 | 5.8 |
| (ODO3921) | | | | | |
| CC from Partial | 12.1 | 19.9 | 20.7 | 19.3 | 27.0 |
| Hepatectomy | | | | | |
| (ODO4309) | | | | | |
| Mets | | | | | |
| Liver Margin | 0.4 | 3.6 | 2.4 | 2.6 | 3.3 |
| (ODO4309) | | | | | |
| Colon mets to | 5.8 | 11.9 | 6.1 | 8.5 | 10.7 |
| lung (OD04451- | | | | | |
| 01) | | | | | |
| Lung Margin | 9.3 | 17.7 | 7.7 | 10.0 | 15.4 |
| (OD04451-02) | | | | | |
| Normal Prostate | 10.5 | 51.1 | 7.3 | 21.6 | 7.0 |
| 6546-1 | | | | | |
| Prostate Cancer | 12.2 | 14.9 | 14.9 | 9.0 | 17.4 |
| (OD04410) | | | | | |
| Prostate Margin | 14.6 | 13.8 | 25.3 | 19.2 | 29.7 |
| (OD04410) | | | | | |
| Prostate Cancer | 12.2 | 18.0 | 22.7 | 31.6 | 30.6 |
| (OD04720-01) | | | | | |
| Prostate Margin | 11.8 | 11.8 | 17.7 | 16.7 | 25.0 |
| (OD04720-02) | | | | | |
| Normal Lung | 7.3 | 17.8 | 17.6 | 12.8 | 22.4 |
| Lung Met to | 12.7 | 27.4 | 25.0 | 31.0 | 22.1 |
| Muscle | | | | | |
| (ODO4286) | | | | | |
| Muscle Margin | 7.4 | 8.7 | 6.2 | 7.3 | 9.5 |
| (ODO4286) | | | | | |
| Lung Malignant | 22.7 | 27.4 | 26.1 | 28.3 | 20.4 |
| Cancer | | | | | |
| (OD03126) | | | | | |
| Lung Margin | 12.7 | 21.9 | 21.9 | 13.9 | 31.9 |
| (OD03126) | | | | | |
| Lung Cancer | 17.9 | 41.5 | 41.5 | 30.4 | 48.0 |
| (OD04404) | | | | | |
| Lung Margin | 16.4 | 28.7 | 10.0 | 11.8 | 12.4 |
| (OD04404) | | | | | |
| Lung Cancer | 22.5 | 38.2 | 28.5 | 27.9 | 40.6 |
| (OD04565) | | | | | |
| Lung Margin | 8.1 | 11.7 | 8.5 | 8.6 | 16.3 |
| (OD04565) | | | | | |
| Lung Cancer | 9.8 | 7.1 | 10.9 | 8.8 | 9.6 |
| (OD04237-01) | | | | | |
| Lung Margin | 12.9 | 23.0 | 14.3 | 14.0 | 16.0 |
| (OD04237-02) | | | | | |
| Ocular Mel Met | 0.6 | 0.5 | 0.7 | 0.5 | 1.1 |
| to Liver | | | | | |
| (ODO4310) | | | | | |
| Liver Margin | 3.5 | 2.6 | 1.8 | 3.3 | 3.0 |
| (ODO4310) | | | | | |
| Melanoma | 1.4 | 2.0 | 3.6 | 4.3 | 2.9 |
| Metastasis | | | | | |
| Lung Margin | 20.4 | 14.4 | 25.2 | 24.0 | 18.6 |
| (OD04321) | | | | | |
| Normal Kidney | 20.2 | 19.9 | 18.0 | 17.4 | 26.1 |
| Kidney Ca, | 1.7 | 4.2 | 2.9 | 2.7 | 4.9 |
| Nuclear grade 2 | | | | | |
| (OD04338) | | | | | |
| Kidney Margin | 6.2 | 11.7 | 17.2 | 11.3 | 22.8 |
| (OD04338) | | | | | |
| Kidney Ca | 3.6 | 10.0 | 3.7 | 4.6 | 6.6 |
| Nuclear grade | | | | | |
| 1/2 (OD04339) | | | | | |
| Kidney Margin | 11.7 | 12.2 | 11.4 | 12.1 | 11.0 |
| (OD04339) | | | | | |
| Kidney Ca, Clear | 46.7 | 50.7 | 66.0 | 65.1 | 70.7 |
| cell type | | | | | |
| (OD04340) | | | | | |
| Kidney Margin | 15.3 | 19.1 | 14.8 | 12.9 | 16.8 |
| (OD04340) | | | | | |
| Kidney Ca, | 21.0 | 9.5 | 16.3 | 16.8 | 17.0 |
| Nuclear grade 3 | | | | | |
| (OD04348) | | | | | |
| Kidney Margin | 8.2 | 5.8 | 8.8 | 11.5 | 9.3 |
| (OD04348) | | | | | |
| Kidney Cancer | 24.0 | 25.3 | 27.7 | 24.8 | 41.5 |
| (OD04622-01) | | | | | |
| Kidney Margin | 2.1 | 4.6 | 3.4 | 3.1 | 5.9 |
| (OD04622-03) | | | | | |
| Kidney Cancer | 0.2 | 0.0 | 0.2 | 0.5 | 0.5 |
| (OD04450-01) | | | | | |
| Kidney Margin | 5.9 | 6.3 | 9.3 | 9.9 | 12.9 |
| (OD04450-03) | | | | | |
| Kidney Cancer | 7.3 | 9.1 | 11.9 | 12.8 | 13.4 |
| 8120607 | | | | | |
| Kidney Margin | 12.2 | 6.2 | 7.9 | 5.6 | 8.0 |
| 8120608 | | | | | |
| Kidney Cancer | 3.6 | 8.0 | 5.2 | 8.8 | 10.1 |
| 8120613 | | | | | |
| Kidney Margin | 6.3 | 6.7 | 8.9 | 7.5 | 9.3 |
| 8120614 | | | | | |
| Kidney Cancer | 18.7 | 61.1 | 25.0 | 21.9 | 22.1 |
| 9010320 | | | | | |
| Kidney Margin | 14.0 | 20.3 | 16.4 | 12.9 | 17.9 |
| 9010321 | | | | | |
| Normal Uterus | 4.1 | 5.6 | 3.3 | 8.4 | 6.0 |
| Uterine Cancer | 9.6 | 10.7 | 17.1 | 11.7 | 15.6 |
| 064011 | | | | | |
| Normal Thyroid | 2.6 | 9.2 | 2.6 | 1.5 | 3.6 |
| Thyroid Cancer | 100.0 | 72.7 | 100.0 | 82.9 | 100.0 |
| Thyroid Cancer | 7.6 | 4.5 | 12.5 | 8.0 | 11.7 |
| A302152 | | | | | |
| Thyroid Margin | 3.0 | 2.4 | 2.8 | 3.2 | 6.0 |
| A302153 | | | | | |
| Normal Breast | 10.3 | 5.7 | 9.9 | 12.9 | 7.2 |
| Breast Cancer | 11.7 | 15.9 | 12.8 | 12.9 | 12.8 |
| Breast Cancer | 17.9 | 39.0 | 27.2 | 16.5 | 25.3 |
| (OD04590-01) | | | | | |
| Breast Cancer | 26.1 | 66.0 | 35.4 | 42.0 | 27.9 |
| Mets (OD04590- | | | | | |
| 03) | | | | | |
| Breast Cancer | 4.5 | 5.4 | 6.0 | 5.2 | 3.5 |
| Metastasis | | | | | |
| Breast Cancer | 30.8 | 32.1 | 28.1 | 21.6 | 36.3 |
| Breast Cancer | 20.7 | 46.7 | 19.8 | 16.7 | 14.8 |
| Breast Cancer | 13.1 | 15.9 | 13.9 | 11.0 | 22.1 |
| 9100266 | | | | | |
| Breast Margin | 10.4 | 14.4 | 15.6 | 16.4 | 20.9 |
| 9100265 | | | | | |
| Breast Cancer | 22.2 | 26.8 | 34.2 | 25.5 | 50.0 |
| A209073 | | | | | |
| Breast Margin | 6.7 | 9.7 | 7.1 | 4.3 | 11.3 |
| A2090734 | | | | | |
| Normal Liver | 1.4 | 4.2 | 1.6 | 1.7 | 2.3 |
| Liver Cancer | 1.0 | 2.8 | 1.7 | 1.3 | 1.3 |
| Liver Cancer | 1.4 | 1.1 | 3.3 | 2.3 | 3.2 |
| 1025 | | | | | |
| Liver Cancer | 7.8 | 6.5 | 4.9 | 6.4 | 10.7 |
| 1026 | | | | | |
| Liver Cancer | 5.0 | 9.9 | 4.2 | 3.0 | 5.2 |
| 6004-T | | | | | |
| Liver Tissue | 4.7 | 7.9 | 3.5 | 4.2 | 3.7 |
| 6004-N | | | | | |
| Liver Cancer | 7.9 | 11.5 | 8.2 | 10.3 | 6.7 |
| 6005-T | | | | | |
| Liver Tissue | 2.0 | 3.2 | 2.7 | 1.6 | 2.3 |
| 6005-N | | | | | |
| Normal Bladder | 6.8 | 17.9 | 13.6 | 11.5 | 15.2 |
| Bladder Cancer | 10.7 | 22.8 | 14.5 | 14.2 | 14.2 |
| Bladder Cancer | 18.0 | 29.3 | 22.7 | 17.7 | 23.5 |
| Bladder Cancer | 14.5 | 29.3 | 26.1 | 21.0 | 28.3 |
| (OD04718-01) | | | | | |
| Bladder Normal | 2.9 | 5.0 | 3.1 | 3.2 | 4.2 |
| Adjacent | | | | | |
| (OD04718-03) | | | | | |
| Normal Ovary | 1.4 | 4.7 | 3.6 | 4.6 | 5.4 |
| Ovarian Cancer | 40.9 | 100.0 | 89.5 | 100.0 | 76.3 |
| Ovarian Cancer | 9.7 | 43.2 | 16.7 | 15.6 | 19.5 |
| (OD04768-07) | | | | | |
| Ovary Margin | 6.5 | 7.9 | 10.8 | 6.7 | 8.3 |
| (OD04768-08) | | | | | |
| Normal Stomach | 11.8 | 39.5 | 14.7 | 14.8 | 13.1 |
| Gastric Cancer | 1.4 | 6.0 | 2.9 | 2.8 | 2.9 |
| 9060358 | | | | | |
| Stomach Margin | 6.4 | 19.9 | 7.4 | 10.8 | 8.7 |
| 9060359 | | | | | |
| Gastric Cancer | 11.1 | 58.6 | 21.6 | 21.2 | 32.3 |
| 9060395 | | | | | |
| Stomach Margin | 6.8 | 34.6 | 23.7 | 13.8 | 22.2 |
| 9060394 | | | | | |
| Gastric Cancer | 15.4 | 78.5 | 24.8 | 25.2 | 31.9 |
| 9060397 | | | | | |
| Stomach Margin | 3.9 | 14.5 | 6.1 | 7.5 | 7.9 |
| 9060396 | | | | | |
| Gastric Cancer | 2.5 | 14.8 | 7.0 | 7.3 | 13.0 |
| 064005 | | | | | |

TABLE 22
Panel 3D

| | Rel. Exp. (%) | | Rel. Exp. (%) |
|---|---|---|---|
| | Ag2263, Run | | Ag2263, Run |
| Tissue Name | 170189128 | Tissue Name | 170189128 |
| Daoy-Medulloblastoma | 19.1 | Ca Ski-Cervical epidermoid | 0.4 |
| | | carcinoma (metastasis) | |
| TE671-Medulloblastoma | 8.4 | ES-2-Ovarian clear cell | 0.0 |
| | | carcinoma | |
| D283 Med- | 39.2 | Ramos-Stimulated with | 0.0 |
| Medulloblastoma | | PMA/ionomycin 6 h | |
| PFSK-1-Primitive | 59.5 | Ramos-Stimulated with | 0.0 |
| Neuroectodermal | | PMA/ionomycin 14 h | |
| XF-498-CNS | 0.9 | MEG-01-Chronic | 3.8 |
| | | myelogenous leukemia | |
| | | (megokaryoblast) | |
| SNB-78-Glioma | 35.4 | Raji-Burkitt's lymphoma | 0.0 |
| SF-268-Glioblastoma | 0.0 | Daudi-Burkitt's lymphoma | 0.0 |
| T98G-Glioblastoma | 1.2 | U266-B-cell plasmacytoma | 0.0 |
| SK-N-SH- | 94.6 | CA46-Burkitt's lymphoma | 0.0 |
| Neuroblastoma | | | |
| (metastasis) | | | |
| SF-295-Glioblastoma | 0.3 | RL-non-Hodgkin's B-cell | 0.0 |
| | | lymphoma | |
| Cerebellum | 37.4 | JM1-pre-B-cell lymphoma | 0.0 |
| Cerebellum | 35.1 | Jurkat-T cell leukemia | 0.5 |
| NCI-H292- | 4.3 | TF-1-Erythroleukemia | 73.2 |
| Mucoepidermoid lung | | | |
| carcinoma | | | |
| DMS-114-Small cell | 6.6 | HUT 78-T-cell lymphoma | 0.0 |
| lung cancer | | | |
| DMS-79-Small cell lung | 100.0 | U937-Histiocytic lymphoma | 0.0 |
| cancer | | | |
| NCI-H146-Small cell | 37.4 | KU-812-Myelogenous | 0.6 |
| lung cancer | | leukemia | |
| NCI-H526-Small cell | 17.2 | 769-P-Clear cell renal | 0.0 |
| lung cancer | | carcinoma | |
| NCI-N417-Small cell | 88.9 | Caki-2-Clear cell renal | 0.0 |
| lung cancer | | carcinoma | |
| NCI-H82-Small cell | 95.3 | SW 839-Clear cell renal | 0.0 |
| lung cancer | | carcinoma | |
| NCI-H157-Squamous | 0.8 | G401-Wilms' tumor | 2.8 |
| cell lung cancer | | | |
| (metastasis) | | | |
| NCI-H1155-Large cell | 55.5 | Hs766T-Pancreatic | 0.6 |
| lung cancer | | carcinoma (LN metastasis) | |
| NCI-H1299-Large cell | 0.0 | CAPAN-1-Pancreatic | 3.1 |
| lung cancer | | adenocarcinoma (liver | |
| | | metastasis) | |
| NCI-H727-Lung | 0.7 | SU86.86-Pancreatic | 0.4 |
| carcinoid | | carcinoma (liver metastasis) | |
| NCI-UMC-11-Lung | 7.9 | BxPC-3-Pancreatic | 22.8 |
| carcinoid | | adenocarcinoma | |
| LX-1-Small cell lung | 1.8 | HPAC-Pancreatic | 35.6 |
| cancer | | adenocarcinoma | |
| Colo-205-Colon cancer | 0.3 | MIA PaCa-2-Pancreatic | 0.6 |
| | | carcinoma | |
| KM12-Colon cancer | 0.1 | CFPAC-1-Pancreatic ductal | 1.1 |
| | | adenocarcinoma | |
| KM20L2-Colon cancer | 0.6 | PANC-1-Pancreatic | 0.3 |
| | | epithelioid ductal carcinoma | |
| NCI-H716-Colon cancer | 70.2 | T24-Bladder carcinma | 0.0 |
| | | (transitional cell) | |
| SW-48-Colon | 0.0 | 5637-Bladder carcinoma | 2.2 |
| adenocarcinoma | | | |
| SW1116-Colon | 16.6 | HT-1197- Bladder carcinoma | 0.4 |
| adenocarcinoma | | | |
| LS 174T-Colon | 4.2 | UM-UC-3-Bladder carcinma | 0.2 |
| adenocarcinoma | | (transitional cell) | |
| SW-948-Colon | 0.4 | A204-Rhabdomyosarcoma | 0.0 |
| adenocarcinoma | | | |
| SW-480-Colon | 0.0 | HT-1080-Fibrosarcoma | 7.9 |
| adenocarcinoma | | | |
| NCI-SNU-5-Gastric | 1.7 | MG-63-Osteosarcoma | 16.3 |
| carcinoma | | | |
| KATO III-Gastric | 17.4 | SK-LMS-1-Leiomyosarcoma | 0.0 |
| carcinoma | | (vulva) | |
| NCI-SNU-16-Gastric | 0.7 | SJRH30-Rhabdomyosarcoma | 3.9 |
| carcinoma | | (met to bone marrow) | |
| NCI-SNU-1-Gastric | 23.0 | A431-Epidermoid carcinoma | 34.9 |
| carcinoma | | | |
| RF-1-Gastric | 0.0 | WM266-4-Melanoma | 0.0 |
| adenocarcinoma | | | |
| RF-48-Gastric | 0.0 | DU 145-Prostate carcinoma | 0.0 |
| adenocarcinoma | | (brain metastasis) | |
| MKN-45-Gastric | 11.5 | MDA-MB-468-Breast | 16.4 |
| carcinoma | | adenocarcinoma | |
| NCI-N87-Gastric | 24.0 | SCC-4-Squamous cell | 0.0 |
| carcinoma | | carcinoma of tongue | |
| OVCAR-5-Ovarian | 3.7 | SCC-9-Squamous cell | 0.0 |
| carcinoma | | carcinoma of tongue | |
| RL95-2-Uterine | 4.6 | SCC-15-Squamous cell | 0.0 |
| carcinoma | | carcinoma of tongue | |
| HelaS3-Cervical | 5.9 | CAL 27-Squamous cell | 7.1 |
| adenocarcinoma | | carcinoma of tongue | |

TABLE 23
Panel 4D

| | Rel. Exp. (%) | Rel. Exp. (%) | Rel. Exp. (%) | Rel. Exp. (%) |
|---|---|---|---|---|
| | Ag1522, Run | Ag1848, Run | Ag2263, Run | Ag2422, Run |
| Tissue Name | 145789191 | 160202841 | 151562852 | 159318890 |
| Secondary Th1 act | 0.0 | 0.1 | 0.0 | 0.2 |
| Secondary Th2 act | 0.0 | 0.0 | 0.0 | 0.0 |
| Secondary Tr1 act | 0.0 | 0.0 | 0.0 | 4.6 |
| Secondary Th1 rest | 0.1 | 0.0 | 0.1 | 0.0 |
| Secondary Th2 rest | 0.0 | 0.0 | 0.0 | 0.0 |
| Secondary Tr1 rest | 0.0 | 0.0 | 0.0 | 0.2 |
| Primary Th1 act | 0.1 | 0.2 | 0.2 | 1.0 |
| Primary Th2 act | 0.1 | 0.2 | 0.1 | 0.3 |
| Primary Tr1 act | 0.2 | 0.5 | 0.0 | 0.6 |
| Primary Th1 rest | 0.0 | 0.0 | 0.0 | 0.0 |
| Primary Th2 rest | 0.0 | 0.0 | 0.0 | 0.0 |
| Primary Tr1 rest | 0.0 | 0.0 | 0.0 | 0.0 |
| CD45RA CD4 | 4.9 | 6.3 | 8.5 | 10.6 |
| lymphocyte act | | | | |
| CD45RO CD4 | 0.0 | 0.0 | 0.0 | 0.0 |
| lymphocyte act | | | | |
| CD8 lymphocyte act | 0.0 | 0.0 | 0.0 | 0.0 |
| Secondary CD8 | 0.0 | 0.0 | 0.0 | 0.0 |
| lymphocyte rest | | | | |
| Secondary CD8 | 0.0 | 0.0 | 0.0 | 0.0 |
| lymphocyte act | | | | |
| CD4 lymphocyte none | 0.0 | 0.0 | 0.0 | 0.0 |
| 2ry Th1/Th2/Tr1_anti- | 0.0 | 0.0 | 0.0 | 0.0 |
| CD95 CH11 | | | | |
| LAK cells rest | 1.8 | 2.7 | 2.0 | 5.8 |
| LAK cells IL-2 | 0.0 | 0.0 | 0.0 | 0.0 |
| LAK cells IL-2 + IL-12 | 0.0 | 0.1 | 0.0 | 0.2 |
| LAK cells IL-2 + IFN | 0.0 | 0.1 | 0.0 | 0.2 |
| gamma | | | | |
| LAK cells IL-2 + IL-18 | 0.0 | 0.4 | 0.0 | 0.1 |
| LAK cells | 1.1 | 1.0 | 1.7 | 2.5 |
| PMA/ionomycin | | | | |
| NK Cells IL-2 rest | 0.0 | 0.1 | 0.0 | 0.0 |
| Two Way MLR 3 day | 0.0 | 0.1 | 0.2 | 0.2 |
| Two Way MLR 5 day | 0.2 | 0.3 | 0.8 | 0.6 |
| Two Way MLR 7 day | 0.5 | 0.2 | 0.1 | 0.3 |
| PBMC rest | 0.0 | 0.0 | 0.1 | 0.0 |
| PBMC PWM | 0.0 | 0.1 | 0.0 | 0.0 |
| PBMC PHA-L | 0.0 | 0.1 | 0.0 | 0.0 |
| Ramos (B cell) none | 0.0 | 0.0 | 0.0 | 0.0 |
| Ramos (B cell) | 0.0 | 0.0 | 0.0 | 0.0 |
| ionomycin | | | | |
| B lymphocytes PWM | 0.2 | 0.0 | 0.0 | 0.0 |
| B lymphocytes CD40L | 0.0 | 0.1 | 0.1 | 0.3 |
| and IL-4 | | | | |
| EOL-1 dbcAMP | 0.2 | 0.2 | 0.4 | 0.0 |
| EOL-1 dbcAMP | 0.1 | 0.4 | 0.2 | 0.6 |
| PMA/ionomycin | | | | |
| Dendritic cells none | 1.4 | 1.1 | 1.0 | 2.8 |
| Dendritic cells LPS | 0.3 | 0.4 | 0.3 | 0.4 |
| Dendritic cells anti- | 2.4 | 3.0 | 3.5 | 6.7 |
| CD40 | | | | |
| Monocytes rest | 0.8 | 0.8 | 0.6 | 1.3 |
| Monocytes LPS | 0.0 | 0.0 | 0.3 | 0.0 |
| Macrophages rest | 1.3 | 1.0 | 0.0 | 2.0 |
| Macrophages LPS | 0.0 | 0.2 | 0.1 | 0.4 |
| HUVEC none | 1.1 | 1.4 | 0.6 | 2.5 |
| HUVEC starved | 4.4 | 4.7 | 2.9 | 6.0 |
| HUVEC IL-1beta | 1.7 | 2.8 | 1.0 | 2.3 |
| HUVEC IFN gamma | 1.6 | 1.4 | 2.5 | 1.9 |
| HUVEC TNF alpha + | 0.3 | 0.3 | 0.5 | 0.5 |
| IFN gamma | | | | |
| HUVEC TNF alpha + | 0.2 | 0.3 | 0.3 | 1.3 |
| IL4 | | | | |
| HUVEC IL-11 | 0.9 | 1.2 | 2.2 | 0.5 |
| Lung Microvascular EC | 2.2 | 6.5 | 2.8 | 6.7 |
| none | | | | |
| Lung Microvascular EC | 12.7 | 11.9 | 8.5 | 15.5 |
| TNFalpha + IL-1beta | | | | |
| Microvascular Dermal | 32.1 | 30.8 | 22.4 | 22.4 |
| EC none | | | | |
| Microvascular Dermal | 16.3 | 16.2 | 8.8 | 14.4 |
| EC TNFalpha + IL- | | | | |
| 1beta | | | | |
| Bronchial epithelium | 24.0 | 31.2 | 15.1 | 50.7 |
| TNFalpha + IL1beta | | | | |
| Small airway epithelium | 8.8 | 5.9 | 6.7 | 12.8 |
| none | | | | |
| Small airway epithelium | 31.9 | 43.5 | 21.0 | 44.8 |
| TNFalpha + IL-1beta | | | | |
| Coronery artery SMC | 27.4 | 28.7 | 8.5 | 35.8 |
| rest | | | | |
| Coronery artery SMC | 12.9 | 21.6 | 27.4 | 17.8 |
| TNFalpha + IL-1beta | | | | |
| Astrocytes rest | 17.1 | 14.9 | 23.8 | 24.3 |
| Astrocytes TNFalpha + | 32.8 | 29.5 | 28.1 | 35.1 |
| IL-1beta | | | | |
| KU-812 (Basophil) rest | 1.0 | 1.8 | 1.3 | 0.7 |
| KU-812 (Basophil) | 1.4 | 3.3 | 2.0 | 3.7 |
| PMA/ionomycin | | | | |
| CCD1106 | 1.4 | 0.2 | 0.7 | 2.7 |
| (Keratinocytes) none | | | | |
| CCD1106 | 0.9 | 0.3 | 0.8 | 1.3 |
| (Keratinocytes) | | | | |
| TNFalpha + IL-1beta | | | | |
| Liver cirrhosis | 2.9 | 3.0 | 2.4 | 4.8 |
| Lupus kidney | 3.0 | 2.9 | 0.9 | 4.4 |
| NCI-H292 none | 10.4 | 13.7 | 5.6 | 18.8 |
| NCI-H292 IL-4 | 14.2 | 14.9 | 6.8 | 17.1 |
| NCI-H292 IL-9 | 13.2 | 16.7 | 9.3 | 12.8 |
| NCI-H292 IL-13 | 9.4 | 8.6 | 15.9 | 9.0 |
| NCI-H292 IFN gamma | 3.8 | 4.7 | 4.7 | 5.3 |
| HPAEC none | 1.2 | 1.0 | 1.6 | 2.8 |
| HPAEC TNF alpha + | 5.8 | 2.6 | 4.7 | 6.0 |
| IL-1 beta | | | | |
| Lung fibroblast none | 100.0 | 100.0 | 100.0 | 100.0 |
| Lung fibroblast TNF | 8.5 | 12.2 | 15.9 | 15.2 |
| alpha + IL-1 beta | | | | |
| Lung fibroblast IL-4 | 74.2 | 79.6 | 45.7 | 97.3 |
| Lung fibroblast IL-9 | 27.7 | 48.6 | 30.6 | 50.3 |
| Lung fibroblast IL-13 | 48.0 | 39.5 | 27.4 | 55.9 |
| Lung fibroblast IFN | 76.3 | 82.9 | 42.6 | 98.6 |
| gamma | | | | |
| Dermal fibroblast | 52.9 | 56.3 | 27.2 | 65.5 |
| CCD1070 rest | | | | |
| Dermal fibroblast | 33.9 | 42.6 | 19.8 | 46.7 |
| CCD1070 TNF alpha | | | | |
| Dermal fibroblast | 29.1 | 27.9 | 70.2 | 28.9 |
| CCD1070 IL-1 beta | | | | |
| Dermal fibroblast IFN | 6.1 | 3.6 | 8.9 | 7.9 |
| gamma | | | | |
| Dermal fibroblast IL-4 | 14.5 | 16.2 | 17.3 | 18.9 |
| IBD Colitis 2 | 0.1 | 0.1 | 0.2 | 0.5 |
| IBD Crohn's | 0.6 | 0.4 | 0.0 | 0.8 |
| Colon | 7.6 | 6.4 | 8.0 | 11.3 |
| Lung | 59.5 | 75.8 | 47.6 | 74.7 |
| Thymus | 16.5 | 17.3 | 10.2 | 19.6 |
| Kidney | 6.8 | 9.0 | 3.0 | 6.5 |

TABLE 24
Panel CNS_1

| | Rel. Exp. (%) Ag2263, | | Rel. Exp. (%) Ag2263, |
|---|---|---|---|
| Tissue Name | Run 171669090 | Tissue Name | Run 171669090 |
| BA4 Control | 22.8 | BA17 PSP | 11.2 |
| BA4 Control2 | 38.2 | BA17 PSP2 | 7.1 |
| BA4 | 3.7 | Sub Nigra Control | 100.0 |
| Alzheimer's2 | | | |
| BA4 Parkinson's | 45.7 | Sub Nigra Control2 | 51.8 |
| BA4 | 31.2 | Sub Nigra | 30.8 |
| Parkinson's2 | | Alzheimer's2 | |
| BA4 | 12.3 | Sub Nigra | 89.5 |
| Huntington's | | Parkinson's2 | |
| BA4 | 12.2 | Sub Nigra | 59.0 |
| Huntington's2 | | Huntington's | |
| BA4 PSP | 13.6 | Sub Nigra | 16.2 |
| | | Huntington's2 | |
| BA4 PSP2 | 42.6 | Sub Nigra PSP2 | 22.5 |
| BA4 Depression | 27.9 | Sub Nigra | 40.6 |
| | | Depression | |
| BA4 | 10.9 | Sub Nigra | 12.8 |
| Depression2 | | Depression2 | |
| BA7 Control | 28.3 | Glob Palladus | 36.1 |
| | | Control | |
| BA7 Control2 | 27.2 | Glob Palladus | 21.3 |
| | | Control2 | |
| BA7 | 5.5 | Glob Palladus | 26.1 |
| Alzheimer's2 | | Alzheimer's | |
| BA7 Parkinson's | 13.2 | Glob Palladus | 11.2 |
| | | Alzheimer's2 | |
| BA7 | 12.8 | Glob Palladus | 73.2 |
| Parkinson's2 | | Parkinson's | |
| BA7 | 14.8 | Glob Palladus | 15.7 |
| Huntington's | | Parkinson's2 | |
| BA7 | 22.2 | Glob Palladus PSP | 15.0 |
| Huntington's | | | |
| BA7 PSP | 29.1 | Glob Palladus PSP2 | 10.4 |
| BA7 PSP2 | 8.9 | Glob Palladus | 28.3 |
| | | Depression | |
| BA7 Depression | 5.4 | Temp Pole Control | 5.4 |
| BA9 Control | 14.3 | Temp Pole Control2 | 25.2 |
| BA9 Control2 | 57.0 | Temp Pole | 10.0 |
| | | Alzheimer's | |
| BA9 Alzheimer's | 5.5 | Temp Pole | 2.5 |
| | | Alzheimer's2 | |
| BA9 | 13.8 | Temp Pole | 15.5 |
| Alzheimer's2 | | Parkinson's | |
| BA9 Parkinson's | 16.2 | Temp Pole | 27.9 |
| | | Parkinson's2 | |
| BA9 | 21.0 | Temp Pole | 22.4 |
| Parkinson's | | Huntington's | |
| BA9 | 21.5 | Temp Pole PSP | 1.3 |
| Huntington's | | | |
| BA9 | 11.9 | Temp Pole PSP2 | 6.4 |
| Huntington's2 | | | |
| BA9 PSP | 27.7 | Temp Pole | 12.3 |
| | | Depression2 | |
| BA9 PSP2 | 5.9 | Cing Gyr Control | 48.3 |
| BA9 Depression | 11.0 | Cing Gyr Control2 | 28.1 |
| BA9 | 9.5 | Cing Gyr | 27.2 |
| Depression2 | | Alzheimer's | |
| BA17 Control | 25.0 | Cing Gyr | 13.1 |
| | | Alzheimer's2 | |
| BA17 Control2 | 45.7 | Cing Gyr Parkinson's | 29.7 |
| BA17 | 6.5 | Cing Gyr | 37.4 |
| Alzheimer's2 | | Parkinson's2 | |
| BA17 | 35.4 | Cing Gyr | 70.7 |
| Parkinson's | | Huntington's | |
| BA17 | 15.3 | Cing Gyr | 32.1 |
| Parkinson's2 | | Huntington's2 | |
| BA17 | 15.5 | Cing Gyr PSP | 42.6 |
| Huntington's | | | |
| BA17 | 8.1 | Cing Gyr PSP2 | 8.3 |
| Huntington's2 | | | |
| BA17 | 26.2 | Cing Gyr Depression | 20.6 |
| Depression | | | |
| BA17 | 59.9 | Cing Gyr | 36.3 |
| Depression2 | | Depression2 | |

CNS_Neurodegeneration_v1.0 Summary: Ag1848/Ag2263/Ag2422

Multiple experiments using different probe/primer sets produce results that are in good agreement. Highest expression of a NOV1 gene is detected in the occipital cortex of a control patient. Significant levels of expression are also detected in the hippocampus, inferior temporal cortex, and the superior temporal cortex of brain tissue from an Alzheimer's patient.

Based on its homology, a NOV1 gene product is most similar to an UNC5H receptor, which as a class is known to act both in axon guidance and neuronal migration during development, as well as in inducing apoptosis (except when stimulated by the ligand netrin-1). Panel CNS_Neurodegeneration_V1.0 shows a moderate increase (1.5 to 2-fold) in the temporal cortex of the Alzheimer's disease brain when compared to non-demented elderly either with or without a high amyloid plaque load [this difference is apparent after scaling the RTQ-PCR data based upon overall RNA amount/quality, and is most apparent on Aq2263]. Thus NOV1 gene represents a protein that differentiates demented and non-demented elderly who have a severe amyloid plaque load, making it an excellent drug target in Alzheimer's disease. The modulation and/or selective stimulation of this receptor may be of use in enhancing or directing compensatory synatogenesis and axon/dendritic outgrowth in response to neuronal death (stroke, head trauma) neurodegeneration (Alzheimer's, Parkinson's, Huntington's, spinocerebellar ataxia, progressive supranuclear palsy) or spinal cord injury. Furthermore, antagonism of this receptor may decrease apoptosis in Alzheimer's disease.

References:

1. Ellezam B, Selles-Navarro I, Manitt C, Kennedy T E, McKerracher L. Expression of netrin-1 and its receptors DCC and UNC-5H2 after axotomy and during regeneration of adult rat retinal ganglion cells. Exp Neurol March;2000, 168(1):105-15

Netrins are a family of chemotropic factors that guide axon outgrowth during development; however, their function in the adult CNS remains to be established. We examined the expression of the netrin receptors DCC and UNC5H2 in adult rat retinal ganglion cells (RGCs) after grafting a peripheral nerve (PN) to the transected optic nerve and following optic nerve transection alone. In situ hybridization revealed that both Dcc and Unc5h2 mRNAs are expressed by normal adult RGCs. In addition, netrin-I was found to be constitutively expressed by RGCs. Quantitative analysis using in situ hybridization demonstrated that both Dcc and Unc5h2 were down-regulated by RGCs following axotomy. In the presence of an attached PN graft, Dcc and Unc5h2 were similarly down-regulated in surviving RGCs regardless of their success in regenerating an axon. Northern blot analysis demonstrated expression of netrin-l in both optic and sciatic nerve, and Western blot analysis revealed the presence of netrin protein in both nerves. Immunohistochemical analysis indicated that netrin protein was closely associated with glial cells in the optic nerve. These results suggest that netrin-1, DCC, and UNC5H2 may contribute to regulating the regenerative capacity of adult RGCs.

2. Braisted J E, Catalano S M, Stimac R, Kennedy T E, Tessier-Lavigne M, Shatz C J, O'Leary DD Netrin-1 promotes thalamic axon growth and is required for proper development of the thalamocortical projection. J Neurosci Aug. 1; 2000 20(15):5792-801

The thalamocortical axon (TCA) projection originates in dorsal thalamus, conveys sensory input to the neocortex, and has a critical role in cortical development. We show that the secreted axon guidance molecule netrin-1 acts in vitro as an attractant and growth promoter for dorsal thalamic axons and is required for the proper development of the TCA projection in vivo. As TCAs approach the hypothalamus, they turn laterally into the ventral telencephalon and extend toward the cortex through a population of netrin-1-expressing cells. DCC and neogenin, receptors implicated in mediating the attractant effects of netrin-1, are expressed in dorsal thalamus, whereas unc5h2 and unc5h3, netrin-1 receptors implicated in repulsion, are not. In vitro, dorsal thalamic axons show biased growth toward a source of netrin-1, which can be abolished by netrin-1-blocking antibodies. Netrin-1 also enhances overall axon outgrowth from explants of dorsal thalamus. The biased growth of dorsal thalamic axons toward the internal capsule zone of ventral telencephalic explants is attenuated, but not significantly, by netrin-1-blocking antibodies, suggesting that it releases another attractant activity for TCAs in addition to netrin-1. Analyses of netrin-1 −/− mice reveal that the TCA projection through the ventral telencephalon is disorganized, their pathway is abnormally restricted, and fewer dorsal thalamic axons reach cortex. These findings demonstrate that netrin-1 promotes the growth of TCAs through the ventral telencephalon and cooperates with other guidance cues to control their pathfinding from dorsal thalamus to cortex.

Panel 1.2 Summary: Ag1522

Expression of a NOV1 gene is highest in CNS cancer cell lines (CT=26.1). Of nine tissue samples derived from CNS cancer cell lines, expression of a NOV1 gene occurs in all samples, with expression high in three samples, moderate in five samples and low in one sample. High expression is also detectable in melanoma cell lines. Significant expression of a NOV1 gene is seen in gastric cancer and all ten samples of lung cancer cell lines in this sample. Thus, expression of a NOV1 gene could be used to distinguish those cancer cell lines from normal tissues. In addition, therapeutic modulation of the expression, or activity of a NOV1 gene product, might be of use in the treatment of melanoma, gastric cancer, lung cancer and brain cancer.

Panel 1.3D Summary: Ag1522/Ag1848/Ag2263/Ag2422

Four experiments using different probe/primer sets on the same tissue panel produce results that are in excellent agreement. In all four experiments, highest expression of a NOV1 gene is detected in CNS cancer cell lines. Expression is also significant in lung cancer and melanoma cell lines and in healthy brain tissue from the hippocampus and thalamus regions. Thus, the expression of a NOV1 gene could be used to distinguish these tissue samples from other samples. Moreover, therapeutic modulation of the expression, or function, of the CG50126-01 gene, through the use of small molecule drugs or antibodies, might be beneficial in the treatment of melanoma, lung cancer and brain cancer.

Among metabolic tissues, there is high expression of a NOV1 gene in adult heart tissue (CT=27.8) and moderate expression in fetal heart, adult and fetal liver, pancreas, adrenal gland, thyroid and pituitary. This widespread expression of a NOV1 gene product in tissues with metabolic function suggests a possible role for a NOV1 gene product in metabolic disorders, including obesity and diabetes.

The UNC5H receptors act both in axon guidance and neuronal migration during development, as well as inducers of apoptosis (except when stimulated by the ligand netrin-1). This panel shows widespread expression of a NOV1 gene in the central nervous system. Please see CNS_neurodegeneration_v 1.0 for discussion of potential utility in the central nervous system.

Panel 2D Summary: Ag1522/Ag1848/Ag2263/Ag2422

Results from multiple experiments with four different probe and primer sets are in very good agreement. In all four experiments, highest expression of a NOV1 gene is detected in thyroid and ovarian cancers (CTs=27-30), with lower expression also seen in most of the other tissues on this panel. Thus, the expression of a NOV1 gene could be used to distinguish ovarian and thyroid cancer cell lines from other tissues. Moreover, therapeutic modulation of the expression this gene, or its function, through the use of small molecule drugs or antibodies, might be of benefit in the treatment of ovarian and thyroid cancer. In addition, experiments with the probe and primer set Ag2263 show differential expression between samples derived from lung cancer and their adjacent normal tissues. Thus, expression of a NOV1 gene could be used to distinguish cancerous lung tissue from normal lung tissue. Moreover, therapeutic modulation of the expression or function of this gene or its protein product, through the use of antibodies or small molecule drugs, might be of benefit in the treatment of lung cancer.

Panel 3D Summary: Ag2263

Expression of a NOV 1 gene occurs at moderate levels across all the tissues in this panel. Highest expression is detected in a small cell lung cancer (CT=30.6) and neuroblastoma (CT=30.7). In addition, significant expression is detected in a cluster of small cell lung cancer lines. Thus, this gene could be used to distinguish lung cancer cell lines from other samples. Moreover, therapeutic modulation of the CG50126-01 gene or its protein product, through the use of small molecule drugs or antibodies might be of benefit in the treatment of small cell lung cancer.

Panel 4D Summary: Ag1522/Ag1848/Ag2263/Ag2422

Experiments using each of the four probe and primer sets that correspond to a NOV1 gene produce results that are in excellent agreement. In all the experiments, expression of a NOV1 gene occurs at moderate to low levels in many of the tissues in the sample. Highest expression in each experiment occurs in lung fibroblasts (CT=29). Moderate expression in lung fibroblasts treated with IL-4 is also consistent among all four experiments (CT=30). Lower expression is also detected in a variety of fibroblasts, endothelial and smooth muscle cells. The expression of a NOV1 gene produces a complex profile; it is upregulated by TNF-alpha in small airway epithelium, but clearly downregulated by the same stimulus in lung fibroblasts. The gene most probably encodes a netrin receptor that may be important in understanding cell migration. Regulation of the protein encoded for by a NOV1 gene could potentially control the progression of keloid formation, emphysema and other conditions in which TNF-alpha and IL-1 beta are present and tissue remodeling may occur.

Panel CNS_{—}1 Summary: Ag2263

Expression of NOV1 is moderate to low across many of the tissues in this panel. Highest expression is detected in the substantia nigra (CT=31.4). Although no disease-specific expression is seen in this panel, the expression profile confirms the expression of this gene in the central nervous system. Please see CNS_neurodegeneration_v1.0 for potential utility of the CG50126-01 gene regarding the CNS.

NOV2

Expression of gene CG50718-01 was assessed using the primer-probe sets Ag1555 and Ag2315, described in Tables 25 and 26. Results of the RTQ-PCR runs are shown in Tables 27, 28, 29 and 30.

TABLE 25

Probe Name Ag1555

| | | | Start | Seq ID |
|---|---|---|---|---|
| Primers | Sequences | Length | Position | NO: |
| Forward | 5′-gaagtgaaagaatgtgcatggt-3′ | 22 | 6680 | 145 |
| Probe | TET-5′-caccagtgcattctggatctcttatca-3′-TAMRA | 27 | 6730 | 146 |
| Reverse | 5′-tgggctgattacttcccttatt-3′ | 22 | 6757 | 147 |

TABLE 26

Probe Name AG2315

| | | | Start | SEQ ID |
|---|---|---|---|---|
| Primers | Sequences | Length | Position | NO: |
| Forward | 5′-agatgagtcagtgccgttagc-3′ | 21 | 3711 | 148 |
| Probe | TET-5′-cctccacaaaatttgactttaatcaactg-3′-TAMRA | 29 | 3733 | 149 |
| Reverse | 5′-tccatttcagccatacaaagtc-3′ | 22 | 3769 | 150 |

TABLE 27
Panel 1.3D

| | Rel. | Rel. | Rel. | | Rel. | Rel. | Rel. |
|---|---|---|---|---|---|---|---|
| | Exp. (%) | Exp. (%) | Exp. (%) | | Exp. (%) | Exp. (%) | Exp. (%) |
| | Ag1555, | Ag1555, | Ag2315, | | Ag1555, | Ag1555, | Ag2315, |
| | Run | Run | Run | Tissue | Run | Run | Run |
| Tissue Name | 146380268 | 147775028 | 159198312 | Name | 146380268 | 147775028 | 159198312 |
| Liver | 0.0 | 0.0 | 0.0 | Kidney | 33.9 | 37.6 | 90.8 |
| adenocarcinoma | | | | (fetal) | | | |
| Pancreas | 5.8 | 1.6 | 3.9 | Renal ca. | 0.0 | 0.0 | 0.0 |
| | | | | 786-0 | | | |
| Pancreatic ca. | 0.0 | 0.0 | 0.0 | Renal ca. | 0.0 | 0.0 | 0.0 |
| CAPAN 2 | | | | A498 | | | |
| Adrenal gland | 0.0 | 1.9 | 0.0 | Renal ca. | 0.0 | 0.0 | 0.0 |
| | | | | RXF 393 | | | |
| Thyroid | 8.3 | 24.7 | 25.3 | Renal ca. | 0.0 | 0.0 | 0.0 |
| | | | | ACHN | | | |
| Salivary gland | 1.0 | 0.0 | 0.0 | Renal ca. | 0.0 | 1.4 | 0.0 |
| | | | | UO-31 | | | |
| Pituitary gland | 0.0 | 0.0 | 8.0 | Renal ca. | 0.0 | 0.0 | 0.0 |
| | | | | TK-10 | | | |
| Brain (fetal) | 0.6 | 0.0 | 3.8 | Liver | 0.0 | 0.0 | 0.0 |
| Brain (whole) | 1.3 | 1.6 | 3.3 | Liver (fetal) | 0.0 | 3.6 | 0.0 |
| Brain | 3.4 | 4.0 | 6.7 | Liver ca. | 0.0 | 0.0 | 0.0 |
| (amygdala) | | | | (hepatoblast) | | | |
| | | | | HepG2 | | | |
| Brain | 0.0 | 0.0 | 0.0 | Lung | 51.1 | 52.5 | 70.7 |
| (cerebellum) | | | | | | | |
| Brain | 1.2 | 0.6 | 5.9 | Lung (fetal) | 100.0 | 100.0 | 74.2 |
| (hippocampus) | | | | | | | |
| Brain | 0.0 | 0.0 | 0.0 | Lung ca. | 0.0 | 0.0 | 0.0 |
| (substantia | | | | (small cell) | | | |
| nigra) | | | | LX-1 | | | |
| Brain | 3.2 | 1.3 | 4.0 | Lung ca. | 2.4 | 0.0 | 32.5 |
| (thalamus) | | | | (small cell) | | | |
| | | | | NCI-H69 | | | |
| Cerebral Cortex | 0.0 | 0.0 | 12.4 | Lung ca. | 0.0 | 0.0 | 10.7 |
| | | | | (s.cell var.) | | | |
| | | | | SHP-77 | | | |
| Spinal cord | 1.1 | 0.0 | 0.0 | Lung ca. | 0.0 | 0.0 | 0.0 |
| | | | | (large | | | |
| | | | | cell)NCI- | | | |
| | | | | H460 | | | |
| glio/astro U87- | 0.0 | 2.7 | 0.0 | Lung ca. | 0.0 | 0.0 | 0.0 |
| MG | | | | (non-sm. | | | |
| | | | | cell) A549 | | | |
| glio/astro U- | 27.2 | 34.6 | 15.8 | Lung ca. | 0.0 | 0.0 | 0.0 |
| 118-MG | | | | (non-s.cell) | | | |
| | | | | NCI-H23 | | | |
| astrocytoma | 5.4 | 13.8 | 16.0 | Lung ca. | 0.7 | 0.9 | 0.0 |
| SW1783 | | | | (non-s.cell) | | | |
| | | | | HOP-62 | | | |
| neuro*; met | 0.0 | 0.6 | 0.0 | Lung ca. | 9.9 | 5.4 | 20.9 |
| SK-N-AS | | | | (non-s.cl) | | | |
| | | | | NCI-H522 | | | |
| astrocytoma SF- | 0.8 | 0.0 | 0.0 | Lung ca. | 0.0 | 0.0 | 0.0 |
| 539 | | | | (squam.) SW | | | |
| | | | | 900 | | | |
| astrocytoma | 0.0 | 0.0 | 0.0 | Lung ca. | 1.3 | 2.2 | 9.0 |
| SNB-75 | | | | (squam.) | | | |
| | | | | NCI-H596 | | | |
| glioma SNB-19 | 0.0 | 0.0 | 0.0 | Mammary | 13.0 | 26.6 | 11.5 |
| | | | | gland | | | |
| glioma U251 | 0.0 | 0.0 | 0.0 | Breast ca.* | 3.9 | 0.9 | 15.6 |
| | | | | (pl.ef) | | | |
| | | | | MCF-7 | | | |
| glioma SF-295 | 1.3 | 3.3 | 0.0 | Breast ca.* | 0.0 | 0.0 | 0.0 |
| | | | | (pl.ef) | | | |
| | | | | MDA-MB- | | | |
| | | | | 231 | | | |
| Heart (Fetal) | 0.0 | 0.0 | 7.4 | Breast ca.* | 0.0 | 0.0 | 6.1 |
| | | | | (pl. ef) | | | |
| | | | | T47D | | | |
| Heart | 0.0 | 5.7 | 0.0 | Breast ca. | 0.0 | 0.0 | 0.0 |
| | | | | BT-549 | | | |
| Skeletal muscle | 3.5 | 1.6 | 15.1 | Breast ca. | 0.0 | 0.0 | 0.0 |
| (Fetal) | | | | MDA-N | | | |
| Skeletal muscle | 0.0 | 1.4 | 2.5 | Ovary | 5.2 | 1.6 | 5.8 |
| Bone marrow | 1.0 | 4.1 | 0.0 | Ovarian ca. | 0.0 | 0.0 | 6.7 |
| | | | | OVCAR-3 | | | |
| Thymus | 1.0 | 0.0 | 8.8 | Ovarian ca. | 0.0 | 0.0 | 0.0 |
| | | | | OVCAR-4 | | | |
| Spleen | 0.0 | 0.0 | 0.0 | Ovarian ca. | 0.0 | 0.0 | 0.0 |
| | | | | OVCAR-5 | | | |
| Lymph node | 3.7 | 4.8 | 7.1 | Ovarian ca. | 0.0 | 0.0 | 0.0 |
| | | | | OVCAR-8 | | | |
| Colorectal | 0.0 | 0.0 | 0.0 | Ovarian ca. | 0.0 | 0.0 | 0.0 |
| | | | | IGROV-1 | | | |
| Stomach | 1.2 | 2.3 | 0.0 | Ovarian ca. | 0.0 | 0.0 | 0.0 |
| | | | | (ascites) SK- | | | |
| | | | | OV-3 | | | |
| Small intestine | 2.2 | 6.7 | 0.0 | Uterus | 0.0 | 0.9 | 0.0 |
| Colon ca. | 0.0 | 0.0 | 0.0 | Placenta | 11.8 | 27.7 | 23.7 |
| SW480 | | | | | | | |
| Colon ca.* | 0.0 | 0.0 | 0.0 | Prostate | 3.5 | 0.9 | 3.0 |
| SW620 (SW480 | | | | | | | |
| met) | | | | | | | |
| Colon ca. HT29 | 0.0 | 0.0 | 0.0 | Prostate ca.* | 0.0 | 0.0 | 0.0 |
| | | | | (bone met) | | | |
| | | | | PC-3 | | | |
| Colon ca. HCT- | 0.0 | 0.0 | 2.7 | Testis | 58.2 | 67.4 | 21.5 |
| 116 | | | | | | | |
| Colon ca. | 0.0 | 0.0 | 0.0 | Melanoma | 22.7 | 52.1 | 18.2 |
| CaCo-2 | | | | Hs688(A).T | | | |
| CC Well to | 0.0 | 0.0 | 0.0 | Melanoma* | 4.8 | 4.2 | 0.0 |
| Mod Diff | | | | (met) | | | |
| (ODO3866) | | | | Hs688(B).T | | | |
| Colon ca. HCC- | 0.0 | 0.0 | 0.0 | Melanoma | 0.0 | 1.5 | 0.0 |
| 2998 | | | | UACC-62 | | | |
| Gastric ca. | 0.0 | 0.0 | 0.0 | Melanoma | 0.0 | 0.0 | 0.0 |
| (liver met) NCI- | | | | M14 | | | |
| N87 | | | | | | | |
| Bladder | 2.0 | 0.0 | 6.1 | Melanoma | 0.0 | 0.0 | 0.0 |
| | | | | LOX IMVI | | | |
| Trachea | 2.4 | 3.6 | 0.0 | Melanoma* | 0.0 | 0.0 | 0.0 |
| | | | | (met) SK- | | | |
| | | | | MEL-5 | | | |
| Kidney | 15.5 | 17.8 | 22.2 | Adipose | 38.2 | 40.6 | 100.0 |

TABLE 28
Panel 2D

| | Rel. | Rel. | Rel. | | Rel. | Rel. | Rel. |
|---|---|---|---|---|---|---|---|
| | Exp. (%) | Exp. (%) | Exp. (%) | | Exp. (%) | Exp. (%) | Exp. (%) |
| | Ag1555, | Ag1555, | Ag2315, | | Ag1555, | Ag1555, | Ag2315, |
| | Run | Run | Run | Tissue | Run | Run | Run |
| Tissue Name | 147775063 | 159601974 | 159200827 | Name | 147775063 | 159601974 | 159200827 |
| Normal | 3.8 | 7.1 | 12.4 | Kidney | 2.9 | 1.2 | 1.7 |
| Colon | | | | Margin | | | |
| | | | | 8120608 | | | |
| CC Well to | 1.0 | 0.0 | 2.3 | Kidney | 0.0 | 0.0 | 0.0 |
| Mod Diff | | | | Cancer | | | |
| (ODO3866) | | | | 8120613 | | | |
| CC Margin | 0.0 | 0.0 | 0.7 | Kidney | 1.2 | 2.6 | 1.8 |
| (ODO3866) | | | | Margin | | | |
| | | | | 8120614 | | | |
| CC Gr.2 | 0.0 | 0.0 | 0.0 | Kidney | 2.7 | 2.6 | 2.1 |
| rectosigmoid | | | | Cancer | | | |
| (ODO3868) | | | | 9010320 | | | |
| CC Margin | 0.0 | 0.7 | 0.0 | Kidney | 6.6 | 5.9 | 4.9 |
| (ODO3868) | | | | Margin | | | |
| | | | | 9010321 | | | |
| CC Mod Diff | 0.0 | 0.0 | 0.0 | Normal | 0.0 | 0.0 | 1.8 |
| (ODO3920) | | | | Uterus | | | |
| CC Margin | 0.0 | 0.0 | 2.2 | Uterine | 0.0 | 0.0 | 4.5 |
| (ODO3920) | | | | Cancer | | | |
| | | | | 064011 | | | |
| CC Gr.2 | 0.0 | 0.0 | 0.0 | Normal | 34.9 | 27.4 | 11.4 |
| ascend colon | | | | Thyroid | | | |
| (ODO3921) | | | | | | | |
| CC Margin | 0.0 | 0.0 | 0.0 | Thyroid | 2.9 | 7.2 | 7.9 |
| (ODO3921) | | | | Cancer | | | |
| CC from | 1.6 | 1.1 | 0.0 | Thyroid | 1.3 | 3.3 | 2.0 |
| Partial | | | | Cancer | | | |
| Hepatectomy | | | | A302152 | | | |
| (ODO4309) | | | | | | | |
| Mets | | | | | | | |
| Liver Margin | 0.0 | 0.0 | 2.0 | Thyroid | 49.7 | 69.7 | 72.2 |
| (ODO4309) | | | | Margin | | | |
| | | | | A302153 | | | |
| Colon mets to | 2.0 | 1.0 | 0.5 | Normal | 10.0 | 8.9 | 25.3 |
| lung | | | | Breast | | | |
| (OD04451- | | | | | | | |
| 01) | | | | | | | |
| Lung Margin | 8.6 | 10.0 | 10.9 | Breast | 10.2 | 3.0 | 1.1 |
| (OD04451- | | | | Cancer | | | |
| 02) | | | | | | | |
| Normal | 4.2 | 12.2 | 1.4 | Breast | 0.0 | 2.8 | 3.9 |
| Prostate | | | | Cancer | | | |
| 6546-1 | | | | (OD04590- | | | |
| | | | | 01) | | | |
| Prostate | 0.0 | 0.0 | 3.4 | Breast | 7.8 | 7.3 | 7.9 |
| Cancer | | | | Cancer | | | |
| (OD04410) | | | | Mets | | | |
| | | | | (OD04590- | | | |
| | | | | 03) | | | |
| Prostate | 0.8 | 6.4 | 2.2 | Breast | 4.1 | 8.0 | 3.5 |
| Margin | | | | Cancer | | | |
| (OD04410) | | | | Metastasis | | | |
| Prostate | 9.5 | 11.7 | 19.6 | Breast | 0.0 | 0.0 | 1.2 |
| Cancer | | | | Cancer | | | |
| (OD04720- | | | | | | | |
| 01) | | | | | | | |
| Prostate | 10.0 | 11.3 | 24.5 | Breast | 3.7 | 2.9 | 0.9 |
| Margin | | | | Cancer | | | |
| (OD04720- | | | | | | | |
| 02) | | | | | | | |
| Normal Lung | 59.9 | 61.1 | 87.7 | Breast | 2.2 | 1.1 | 1.5 |
| | | | | Cancer | | | |
| | | | | 9100266 | | | |
| Lung Met to | 0.0 | 0.0 | 0.0 | Breast | 0.0 | 0.0 | 0.5 |
| Muscle | | | | Margin | | | |
| (ODO4286) | | | | 9100265 | | | |
| Muscle | 0.9 | 0.0 | 1.8 | Breast | 0.7 | 1.1 | 1.9 |
| Margin | | | | Cancer | | | |
| (ODO4286) | | | | A209073 | | | |
| Lung | 1.9 | 2.8 | 1.7 | Breast | 0.0 | 1.2 | 0.9 |
| Malignant | | | | Margin | | | |
| Cancer | | | | A2090734 | | | |
| (OD03126) | | | | | | | |
| Lung Margin | 36.3 | 35.6 | 43.8 | Normal | 0.0 | 0.0 | 0.0 |
| (OD03126) | | | | Liver | | | |
| Lung Cancer | 2.2 | 4.4 | 4.3 | Liver | 0.0 | 0.0 | 0.6 |
| (OD04404) | | | | Cancer | | | |
| Lung Margin | 9.5 | 4.2 | 8.4 | Liver | 0.0 | 0.0 | 0.0 |
| (OD04404) | | | | Cancer | | | |
| | | | | 1025 | | | |
| Lung Cancer | 0.0 | 0.0 | 0.0 | Liver | 0.0 | 0.0 | 0.0 |
| (OD04565) | | | | Cancer | | | |
| | | | | 1026 | | | |
| Lung Margin | 10.8 | 9.7 | 14.1 | Liver | 0.0 | 1.0 | 0.6 |
| (OD04565) | | | | Cancer | | | |
| | | | | 6004-T | | | |
| Lung Cancer | 0.0 | 0.0 | 0.0 | Liver | 0.0 | 0.0 | 0.0 |
| (OD04237- | | | | Tissue | | | |
| 01) | | | | 6004-N | | | |
| Lung Margin | 30.1 | 18.4 | 29.3 | Liver | 0.0 | 0.0 | 0.0 |
| (OD04237- | | | | Cancer | | | |
| 02) | | | | 6005-T | | | |
| Ocular Mel | 0.0 | 0.0 | 0.6 | Liver | 0.0 | 0.0 | 0.0 |
| Met to Liver | | | | Tissue | | | |
| (ODO4310) | | | | 6005-N | | | |
| Liver Margin | 1.0 | 2.0 | 0.0 | Normal | 4.7 | 2.2 | 2.9 |
| (ODO4310) | | | | Bladder | | | |
| Melanoma | 0.0 | 0.0 | 0.0 | Bladder | 0.0 | 0.0 | 0.0 |
| Metastasis | | | | Cancer | | | |
| Lung Margin | 25.7 | 47.0 | 49.0 | Bladder | 0.0 | 4.2 | 5.5 |
| (OD04321) | | | | Cancer | | | |
| Normal | 86.5 | 100.0 | 100.0 | Bladder | 0.7 | 1.6 | 1.1 |
| Kidney | | | | Cancer | | | |
| | | | | (OD04718- | | | |
| | | | | 01) | | | |
| Kidney Ca, | 2.2 | 0.0 | 1.1 | Bladder | 4.4 | 0.9 | 6.3 |
| Nuclear grade | | | | Normal | | | |
| 2 (OD04338) | | | | Adjacent | | | |
| | | | | (OD04718- | | | |
| | | | | 03) | | | |
| Kidney | 55.1 | 35.8 | 58.2 | Normal | 1.7 | 0.0 | 0.9 |
| Margin | | | | Ovary | | | |
| (OD04338) | | | | | | | |
| Kidney Ca | 0.0 | 0.0 | 0.0 | Ovarian | 0.0 | 4.2 | 3.3 |
| Nuclear grade | | | | Cancer | | | |
| 1/2 | | | | | | | |
| (OD04339) | | | | | | | |
| Kidney | 77.9 | 63.7 | 77.9 | Ovarian | 0.0 | 0.0 | 0.0 |
| Margin | | | | Cancer | | | |
| (OD04339) | | | | (OD04768- | | | |
| | | | | 07) | | | |
| Kidney Ca, | 1.7 | 0.0 | 0.0 | Ovary | 9.4 | 5.5 | 6.9 |
| Clear cell | | | | Margin | | | |
| type | | | | (OD04768- | | | |
| (OD04340) | | | | 08) | | | |
| Kidney | 100.0 | 53.2 | 62.4 | Normal | 0.0 | 0.0 | 0.0 |
| Margin | | | | Stomach | | | |
| (OD04340) | | | | | | | |
| Kidney Ca, | 25.9 | 23.2 | 0.0 | Gastric | 0.0 | 0.0 | 1.5 |
| Nuclear grade | | | | Cancer | | | |
| 3 (OD04348) | | | | 9060358 | | | |
| Kidney | 40.9 | 50.3 | 54.7 | Stomach | 0.0 | 0.0 | 2.0 |
| Margin | | | | Margin | | | |
| (OD04348) | | | | 9060359 | | | |
| Kidney | 0.6 | 0.0 | 0.0 | Gastric | 0.9 | 1.2 | 1.8 |
| Cancer | | | | Cancer | | | |
| (OD04622- | | | | 9060395 | | | |
| 01) | | | | | | | |
| Kidney | 0.0 | 0.0 | 1.4 | Stomach | 0.0 | 1.0 | 0.7 |
| Margin | | | | Margin | | | |
| (OD04622- | | | | 9060394 | | | |
| 03) | | | | | | | |
| Kidney | 0.0 | 0.0 | 0.0 | Gastric | 0.0 | 0.0 | 0.0 |
| Cancer | | | | Cancer | | | |
| (OD04450- | | | | 9060397 | | | |
| 01) | | | | | | | |
| Kidney | 40.3 | 51.1 | 50.7 | Stomach | 0.0 | 0.0 | 0.0 |
| Margin | | | | Margin | | | |
| (OD04450- | | | | 9060396 | | | |
| 03) | | | | | | | |
| Kidney | 0.0 | 0.0 | 0.0 | Gastric | 0.0 | 0.0 | 2.5 |
| Cancer | | | | Cancer | | | |
| 8120607 | | | | 064005 | | | |

TABLE 29
Panel 4D

| | Rel. Exp. (%) | Rel. Exp. (%) | | Rel. Exp. (%) | Rel. Exp. (%) |
|---|---|---|---|---|---|
| | Ag1555, Run | Ag2315, Run | | Ag1555, Run | Ag2315, Run |
| Tissue Name | 147775116 | 159202089 | Tissue Name | 147775116 | 159202089 |
| Secondary Th1 act | 0.0 | 0.0 | HUVEC IL-1beta | 0.0 | 0.0 |
| Secondary Th2 act | 0.0 | 0.0 | HUVEC IFN | 0.0 | 0.0 |
| | | | gamma | | |
| Secondary Tr1 act | 0.0 | 0.7 | HUVEC TNF | 0.0 | 0.0 |
| | | | alpha + IFN gamma | | |
| Secondary Th1 rest | 0.0 | 0.0 | HUVEC TNF | 0.0 | 0.0 |
| | | | alpha + IL4 | | |
| Secondary Th2 rest | 0.0 | 0.0 | HUVEC IL-11 | 0.0 | 0.0 |
| Secondary Tr1 rest | 0.0 | 0.0 | Lung Microvascular | 0.0 | 0.0 |
| | | | EC none | | |
| Primary Th1 act | 0.0 | 0.0 | Lung Microvascular | 0.0 | 0.0 |
| | | | EC TNFalpha + IL- | | |
| | | | 1beta | | |
| Primary Th2 act | 0.0 | 0.0 | Microvascular | 0.0 | 0.0 |
| | | | Dermal EC none | | |
| Primary Tr1 act | 0.0 | 0.0 | Microsvasular | 0.0 | 0.0 |
| | | | Dermal EC | | |
| | | | TNFalpha + IL- | | |
| | | | 1beta | | |
| Primary Th1 rest | 0.0 | 0.0 | Bronchial | 0.0 | 0.0 |
| | | | epithelium | | |
| | | | TNFalpha + | | |
| | | | IL1beta | | |
| Primary Th2 rest | 0.0 | 0.0 | Small airway | 0.0 | 0.0 |
| | | | epithelium none | | |
| Primary Tr1 rest | 0.0 | 0.0 | Small airway | 0.0 | 0.0 |
| | | | epithelium | | |
| | | | TNFalpha + IL- | | |
| | | | 1beta | | |
| CD45RA CD4 | 3.3 | 5.0 | Coronery artery | 1.0 | 2.3 |
| lymphocyte act | | | SMC rest | | |
| CD45RO CD4 | 0.0 | 0.0 | Coronery artery | 3.7 | 1.2 |
| lymphocyte act | | | SMC TNFalpha + | | |
| | | | IL-1beta | | |
| CD8 lymphocyte act | 0.0 | 0.0 | Astrocytes rest | 3.2 | 0.5 |
| Secondary CD8 | 0.0 | 0.0 | Astrocytes | 1.0 | 1.5 |
| lymphocyte rest | | | TNFalpha + IL- | | |
| | | | 1beta | | |
| Secondary CD8 | 0.0 | 0.0 | KU-812 (Basophil) | 0.0 | 0.0 |
| lymphocyte act | | | rest | | |
| CD4 lymphocyte | 0.0 | 0.0 | KU-812 (Basophil) | 0.0 | 0.0 |
| none | | | PMA/ionomycin | | |
| 2ry | 0.0 | 0.0 | CCD1106 | 0.0 | 0.0 |
| Th1/Th2/Tr1_anti- | | | (Keratinocytes) | | |
| CD95 CH11 | | | none | | |
| LAK cells rest | 0.0 | 0.0 | CCD1106 | 0.0 | 0.0 |
| | | | (Keratinocytes) | | |
| | | | TNFalpha + IL- | | |
| | | | 1beta | | |
| LAK cells IL-2 | 0.0 | 0.0 | Liver cirrhosis | 1.4 | 3.8 |
| LAK cells IL-2 + IL- | 0.0 | 0.0 | Lupus kidney | 0.0 | 0.8 |
| 12 | | | | | |
| LAK cells IL-2 + IFN | 0.0 | 0.0 | NCI-H292 none | 0.0 | 0.0 |
| gamma | | | | | |
| LAK cells IL-2 + IL- | 0.0 | 0.0 | NCI-H292 IL-4 | 0.0 | 2.3 |
| 18 | | | | | |
| LAK cells | 0.0 | 0.0 | NCI-H292 IL-9 | 0.0 | 0.5 |
| PMA/ionomycin | | | | | |
| NK Cells IL-2 rest | 0.0 | 0.0 | NCI-H292 IL-13 | 0.0 | 1.3 |
| Two Way MLR 3 | 0.0 | 0.0 | NCI-H292 IFN | 0.0 | 0.0 |
| day | | | gamma | | |
| Two Way MLR 5 | 0.0 | 0.0 | HPAEC none | 0.0 | 0.0 |
| day | | | | | |
| Two Way MLR 7 | 0.0 | 0.0 | HPAEC TNF | 0.0 | 0.0 |
| day | | | alpha + IL-1 beta | | |
| PBMC rest | 0.0 | 0.0 | Lung fibroblast | 0.0 | 0.9 |
| | | | none | | |
| PBMC PWM | 0.0 | 0.0 | Lung fibroblast | 0.0 | 0.0 |
| | | | TNF alpha + IL-1 | | |
| | | | beta | | |
| PBMC PHA-L | 0.0 | 0.0 | Lung fibroblast IL-4 | 0.0 | 0.0 |
| Ramos (B cell) none | 0.0 | 0.0 | Lung fibroblast IL-9 | 5.7 | 1.3 |
| Ramos (B cell) | 0.0 | 0.0 | Lung fibroblast IL- | 1.5 | 1.5 |
| ionomycin | | | 13 | | |
| B lymphocytes | 0.0 | 0.0 | Lung fibroblast IFN | 0.0 | 1.7 |
| PWM | | | gamma | | |
| B lymphocytes | 0.0 | 0.0 | Dermal fibroblast | 12.9 | 17.2 |
| CD40L and IL-4 | | | CCD1070 rest | | |
| EOL-1 dbcAMP | 0.0 | 0.0 | Dermal fibroblast | 18.6 | 12.0 |
| | | | CCD1070 TNF | | |
| | | | alpha | | |
| EOL-1 dbcAMP | 0.0 | 0.0 | Dermal fibroblast | 6.1 | 2.9 |
| PMA/ionomycin | | | CCD1070 IL-1 beta | | |
| Dendritic cells none | 0.0 | 0.0 | Dermal fibroblast | 0.0 | 0.0 |
| | | | IFN gamma | | |
| Dendritic cells LPS | 0.0 | 0.0 | Dermal fibroblast | 1.4 | 0.6 |
| | | | IL-4 | | |
| Dendritic cells anti- | 0.0 | 0.0 | IBD Colitis 2 | 0.0 | 1.4 |
| CD40 | | | | | |
| Monocytes rest | 0.0 | 0.0 | IBD Crohn's | 0.0 | 0.0 |
| Monocytes LPS | 0.0 | 0.0 | Colon | 0.6 | 0.0 |
| Macrophages rest | 0.0 | 0.0 | Lung | 4.0 | 11.7 |
| Macrophages LPS | 0.0 | 0.0 | Thymus | 100.0 | 100.0 |
| HUVEC none | 0.0 | 0.0 | Kidney | 4.2 | 5.3 |
| HUVEC starved | 0.0 | 0.0 | | | |

TABLE 30
Panel 5D

| | Rel. Exp. (%) | | Rel. Exp. (%) |
|---|---|---|---|
| | Ag2315, Run | | Ag2315, Run |
| Tissue Name | 169275446 | Tissue Name | 169275446 |
| 97457_Patient- | 84.1 | 94709_Donor 2 AM-A_adipose | 13.6 |
| 02go_adipose | | | |
| 97476_Patient- | 0.6 | 94710_Donor 2 AM-B_adipose | 9.3 |
| 07sk_skeletal muscle | | | |
| 97477_Patient- | 0.0 | 94711_Donor 2 AM-C_adipose | 3.6 |
| 07ut_uterus | | | |
| 97478_Patient- | 7.2 | 94712_Donor 2 AD-A_adipose | 8.7 |
| 07pl_placenta | | | |
| 97481_Patient- | 4.4 | 94713_Donor 2 AD-B_adipose | 17.1 |
| 08sk_skeletal muscle | | | |
| 97482_Patient- | 0.5 | 94714_Donor 2 AD-C_adipose | 21.6 |
| 08ut_uterus | | | |
| 97483_Patient- | 6.5 | 94742_Donor 3 U - | 9.0 |
| 08pl_placenta | | A_Mesenchymal Stem Cells | |
| 97486_Patient- | 0.0 | 94743_Donor 3 U - | 7.3 |
| 09sk_skeletal muscle | | B_Mesenchymal Stem Cells | |
| 97487_Patient- | 0.5 | 94730_Donor 3 AM-A_adipose | 14.8 |
| 09ut_uterus | | | |
| 97488_Patient- | 6.1 | 94731_Donor 3 AM-B_adipose | 13.9 |
| 09pl_placenta | | | |
| 97492_Patient- | 0.0 | 94732_Donor 3 AM-C_adipose | 5.9 |
| 10ut_uterus | | | |
| 97493_Patient- | 7.8 | 94733_Donor 3 AD-A_adipose | 5.4 |
| 10pl_placenta | | | |
| 97495_Patient- | 100.0 | 94734_Donor 3 AD-B_adipose | 4.7 |
| 11go_adipose | | | |
| 97496_Patient- | 0.6 | 94735_Donor 3 AD-C_adipose | 9.3 |
| 11sk_skeletal muscle | | | |
| 97497_Patient- | 1.0 | 77138_Liver_HepG2untreated | 6.9 |
| 11ut_uterus | | | |
| 97498_Patient- | 7.3 | 73556_Heart_Cardiac stromal cells | 0.0 |
| 11pl_placenta | | (primary) | |
| 97500_Patient- | 61.6 | 81735_Small Intestine | 1.5 |
| 12go_adipose | | | |
| 97501_Patient- | 3.2 | 72409_Kidney_Proximal | 0.0 |
| 12sk_skeletal muscle | | Convoluted Tubule | |
| 97502_Patient- | 1.4 | 82685_Small intestine_Duodenum | 0.0 |
| 12ut_uterus | | | |
| 97503_Patient- | 1.5 | 90650_Adrenal_Adrenocortical | 0.0 |
| 12pl_placenta | | adenoma | |
| 94721_Donor 2 U - | 14.4 | 72410_Kidney_HRCE | 0.0 |
| A_Mesenchymal Stem | | | |
| Cells | | | |
| 94722_Donor 2 U - | 6.7 | 72411_Kidney_HRE | 0.0 |
| B_Mesenchymal Stem | | | |
| Cells | | | |
| 94723_Donor 2 U - | 6.0 | 73139_Uterus_Uterine smooth | 0.0 |
| C_Mesenchymal Stem | | muscle cells | |
| Cells | | | |

Panel 1.3D Summary: Ag1555/2315 Highest expression of the CG50718-01 gene is seen in adipose and the fetal lung (CTs=31.8-34.4). Results from three experiments with two different probe and primer sets produce similar expression profiles. Low but significant expression is also seen in the thyroid. Biologic cross-talk between the thyroid and adipose tissue is believed to be a component of some forms of obesity. Thus, the CG50718-01 gene product may be an important small molecule target for the treatment of obesity or other metabolic disorders.

In addition, the CG50718-01 gene appears to be expressed at significant levels in lung and kidney tissues from both fetal and adult sources, but not in any samples derived from lung or kidney cancer cell lines. Thus, expression of this gene could potentially be used to differentiate between normal lung and kidney tissue and lung and kidney cancer. Furthermore, therapeutic modulation of the CG50718-01 gene product may be beneficial in the treatment of lung and kidney cancers.

Please note that two other experiments with the probe and primer set Ag2315 had low/undetectable levels of expression in all the samples on this panel. (Data not shown.)

Panel 2D Summary: Ag1555/2315 Three experiments with two different probe and primer sets produce results that are in excellent agreement with highest expression of the CG50718-01 gene in normal kidney tissue (CTs=30.7-32.4). There are also significant levels of expression in samples derived from normal lung tissue, a result that is in concordance with the expression seen in Panel 1.3D. This gene appears to be preferentially expressed in healthy tissue, when compared to adjacent cancerous tissue. Thus, expression of the CG50718-01 gene could be used to distinguish normal kideny and lung tissue from malignant kidney and lung tissue. Moreover, therapeutic modulation of this gene, through small molecule drugs, antibodies or protein therapeutics might be of benefit in the treatment of kidney cancer and lung cancer.

Panel 3D Summary: Ag2315 Expression is low/undetectable in all the samples in this panel (CT>35). (Data not shown.)

Panel 4D Summary: Ag1555/Ag2315 The CG50718-01 transcript is detected at significant levels in the thymus (CT 31.48) and at lower levels in dermal fibroblasts (CT 33.91). This transcript encodes a protein that could potentially serve as a marker for thymus tissue and may also be involved in skin homeostasis. Therapeutics designed with the protein encoded by the CG50718-01 transcript could be important for maintaining or restoring normal function to these organs during inflammation.

Panel 5D Summary: Ag2315 is modestly expressed (CT values 31-34) in human adipose tissue and in cultured human adipocytes. This expression is in agreement with the significant levels of expression in adipose detected in Panel 1.3D. Thus, this gene product may be a small molecule target for the treatment of obesity.

NOV3

Expression of NOV3 was assessed using the primer-probe set Ag2304, described in Table 31. Results of the RTQ-PCR runs are shown in Tables 32, 33, 34 and 35.

TABLE 31

Probe Name Ag2304

| | | | Start | SEQ ID |
|---|---|---|---|---|
| Primers | Sequences | Length | Position | NO: |
| Forward | 5′-accttaagtcctgccaacaatt-3′ | 22 | 4100 | 151 |
| Probe | TET-5′-ttacagagtccaaaattgtggatccca-3′-TAMRA | 26 | 4147 | 152 |
| Reverse | 5′-tgatcccttccagaatttgac-3′ | 21 | 4173 | 153 |

TABLE 32
CNS_neurodegeneration_v1.0

| | Rel. Exp. | | Rel. Exp. |
|---|---|---|---|
| | (%) Ag2304, | | (%) Ag2304, |
| | Run | | Run |
| Tissue Name | 206262286 | Tissue Name | 206262286 |
| AD 1 Hippo | 0.0 | Control (Path) 3 | 7.3 |
| | | Temporal Ctx | |
| AD 2 Hippo | 38.4 | Control (Path) 4 | 29.5 |
| | | Temporal Ctx | |
| AD 3 Hippo | 8.5 | AD 1 Occipital Ctx | 16.5 |
| AD 4 Hippo | 9.5 | AD 2 Occipital Ctx | 0.0 |
| | | (Missing) | |
| AD 5 Hippo | 100.0 | AD 3 Occipital Ctx | 8.7 |
| AD 6 Hippo | 70.7 | AD 4 Occipital Ctx | 22.2 |
| Control 2 Hippo | 44.8 | AD 5 Occipital Ctx | 45.1 |
| Control 4 Hippo | 13.3 | AD 5 Occipital Ctx | 0.0 |
| Control (Path) 3 | 0.0 | Control 1 Occipital | 4.5 |
| Hippo | | Ctx | |
| AD 1 Temporal Ctx | 25.0 | Control 2 Occipital | 58.2 |
| | | Ctx | |
| AD 2 Temporal Ctx | 39.2 | Control 3 Occipital | 18.2 |
| | | Ctx | |
| AD 3 Temporal Ctx | 7.7 | Control 4 Occipital | 7.3 |
| | | Ctx | |
| AD 4 Temporal Ctx | 0.2 | Control (Path) 1 | 92.7 |
| | | Occipital Ctx | |
| AD 5 Inf Temporal | 76.8 | Control (Path) 2 | 0.0 |
| Ctx | | Occipital Ctx | |
| AD 5 Sup Temporal | 40.6 | Control (Path) 3 | 3.4 |
| Ctx | | Occipital Ctx | |
| AD 6 Inf Temporal | 49.7 | Control (Path) 4 | 16.7 |
| Ctx | | Occipital Ctx | |
| AD 6 Sup Temporal | 57.4 | Control 1 Parietal Ctx | 7.1 |
| Ctx | | | |
| Control 1 Temporal | 9.2 | Control 2 Parietal Ctx | 41.8 |
| Ctx | | | |
| Control 2 Temporal | 40.9 | Control 3 Parietal Ctx | 0.0 |
| Ctx | | | |
| Control 3 Temporal | 20.6 | Control (Path) 1 | 97.9 |
| Ctx | | Parietal Ctx | |
| Control 3 Temporal | 10.7 | Control (Path) 2 | 29.5 |
| Ctx | | Parietal Ctx | |
| Control (Path) 1 | 97.3 | Control (Path) 3 | 4.4 |
| Temporal Ctx | | Parietal Ctx | |
| Control (Path) 2 | 52.9 | Control (Path) 4 | 68.3 |
| Temporal Ctx | | Parietal Ctx | |

TABLE 33
Panel 1.3D

| | Rel. Exp. (%) Ag2304, | | Rel. Exp. (%) Ag2304, |
|---|---|---|---|
| Tissue Name | Run 159131830 | Tissue Name | Run 159131830 |
| Liver adenocarcinoma | 6.0 | Kidney (fetal) | 8.5 |
| Pancreas | 1.7 | Renal ca. 786-0 | 7.6 |
| Pancreatic ca. CAPAN 2 | 2.4 | Renal ca. A498 | 13.2 |
| Adrenal gland | 14.9 | Renal ca. RXF 393 | 3.2 |
| Thyroid | 6.5 | Renal ca. ACHN | 3.1 |
| Salivary gland | 2.3 | Renal ca. UO-31 | 8.3 |
| Pituitary gland | 13.4 | Renal ca. TK-10 | 3.5 |
| Brain (fetal) | 7.7 | Liver | 2.8 |
| Brain (whole) | 13.5 | Liver (fetal) | 5.8 |
| Brain (amygdala) | 15.5 | Liver ca. (hepatoblast) | 7.3 |
| | | HepG2 | |
| Brain (cerebellum) | 4.6 | Lung | 19.9 |
| Brain (hippocampus) | 100.0 | Lung (fetal) | 9.9 |
| Brain (substantia nigra) | 2.8 | Lung ca. (small cell) | 5.4 |
| | | LX-1 | |
| Brain (thalamus) | 10.0 | Lung ca. (small cell) | 12.3 |
| | | NCI-H69 | |
| Cerebral Cortex | 25.0 | Lung ca. (s.cell var.) | 12.1 |
| | | SHP-77 | |
| Spinal cord | 4.0 | Lung ca. (large | 3.8 |
| | | cell)NCI-H460 | |
| glio/astro U87-MG | 21.9 | Lung ca. (non-sm. cell) | 5.9 |
| | | A549 | |
| glio/astro U-118-MG | 40.9 | Lung ca. (non-s.cell) | 13.6 |
| | | NCI-H23 | |
| astrocytoma SW1783 | 9.2 | Lung ca. (non-s.cell) | 7.0 |
| | | HOP-62 | |
| neuro*; met SK-N-AS | 65.5 | Lung ca. (non-s.cl) | 3.4 |
| | | NCI-H522 | |
| astrocytoma SF-539 | 9.8 | Lung ca. (squam.) SW | 6.6 |
| | | 900 | |
| astrocytoma SNB-75 | 11.9 | Lung ca. (squam.) | 1.7 |
| | | NCI-H596 | |
| glioma SNB-19 | 9.6 | Mammary gland | 18.4 |
| glioma U251 | 6.0 | Breast ca.* (pl.ef) | 6.3 |
| | | MCF-7 | |
| glioma SF-295 | 6.5 | Breast ca.* (pl.ef) | 34.6 |
| | | MDA-MB-231 | |
| Heart (Fetal) | 0.6 | Breast ca.* (pl.ef) | 5.1 |
| | | T47D | |
| Heart | 2.3 | Breast ca. BT-549 | 20.2 |
| Skeletal muscle (Fetal) | 11.4 | Breast ca. MDA-N | 5.7 |
| Skeletal muscle | 8.5 | Ovary | 5.6 |
| Bone marrow | 8.5 | Ovarian ca. OVCAR-3 | 7.7 |
| Thymus | 7.4 | Ovarian ca. OVCAR-4 | 0.7 |
| Spleen | 12.0 | Ovarian ca. OVCAR-5 | 16.7 |
| Lymph node | 6.3 | Ovarian ca. OVCAR-8 | 8.6 |
| Colorectal | 4.6 | Ovarian ca. IGROV-1 | 2.4 |
| Stomach | 8.5 | Ovarian ca. (ascites) | 15.5 |
| | | SK-OV-3 | |
| Small intestine | 9.2 | Uterus | 6.4 |
| Colon ca. SW480 | 8.0 | Placenta | 8.1 |
| Colon ca.* SW620 | 5.3 | Prostate | 3.4 |
| (SW480 met) | | | |
| Colon ca. HT29 | 2.6 | Prostate ca.* (bone | 5.9 |
| | | met) PC-3 | |
| Colon ca. HCT-116 | 7.4 | Testis | 18.6 |
| Colon ca. CaCo-2 | 7.4 | Melanoma Hs688(A).T | 4.5 |
| CC Well to Mod Diff | 9.2 | Melanoma* (met) | 2.5 |
| (ODO3866) | | Hs688(B).T | |
| Colon ca. HCC-2998 | 8.6 | Melanoma UACC-62 | 1.6 |
| Gastric ca. (liver met) | 30.8 | Melanoma M14 | 0.6 |
| NCI-N87 | | | |
| Bladder | 2.7 | Melanoma LOX IMVI | 4.0 |
| Trachea | 12.0 | Melanoma* (met) SK- | 2.3 |
| | | MEL-5 | |
| Kidney | 3.5 | Adipose | 7.5 |

TABLE 34
Panel 2D

| | Rel. Exp. (%) Ag2304, | | Rel. Exp. (%) Ag2304, |
|---|---|---|---|
| Tissue Name | Run 159134494 | Tissue Name | Run 159134494 |
| Normal Colon | 82.9 | Kidney Margin | 5.5 |
| | | 8120608 | |
| CC Well to Mod Diff | 21.3 | Kidney Cancer 8120613 | 17.9 |
| (ODO3866) | | | |
| CC Margin (ODO3866) | 14.6 | Kidney Margin | 13.1 |
| | | 8120614 | |
| CC Gr.2 rectosigmoid | 10.9 | Kidney Cancer 9010320 | 24.7 |
| (ODO3868) | | | |
| CC Margin (ODO3868) | 9.9 | Kidney Margin | 19.3 |
| | | 9010321 | |
| CC Mod Diff (ODO3920) | 21.5 | Normal Uterus | 17.6 |
| CC Margin (ODO3920) | 27.4 | Uterine Cancer 064011 | 52.5 |
| CC Gr.2 ascend colon | 45.1 | Normal Thyroid | 22.7 |
| (ODO3921) | | | |
| CC Margin (ODO3921) | 15.8 | Thyroid Cancer | 36.1 |
| CC from Partial | 37.9 | Thyroid Cancer | 18.2 |
| Hepatectomy (ODO4309) | | A302152 | |
| Mets | | | |
| Liver Margin (ODO4309) | 28.9 | Thyroid Margin | 30.1 |
| | | A302153 | |
| Colon mets to lung | 23.2 | Normal Breast | 49.7 |
| (OD04451-01) | | | |
| Lung Margin (OD04451-02) | 24.1 | Breast Cancer | 28.5 |
| Normal Prostate 6546-1 | 18.4 | Breast Cancer | 51.8 |
| | | (OD04590-01) | |
| Prostate Cancer (OD04410) | 59.9 | Breast Cancer Mets | 64.6 |
| | | (OD04590-03) | |
| Prostate Margin (OD04410) | 67.4 | Breast Cancer | 47.6 |
| | | Metastasis | |
| Prostate Cancer (OD04720- | 46.7 | Breast Cancer | 26.2 |
| 01) | | | |
| Prostate Margin (OD04720- | 93.3 | Breast Cancer | 28.9 |
| 02) | | | |
| Normal Lung | 100.0 | Breast Cancer 9100266 | 20.2 |
| Lung Met to Muscle | 41.2 | Breast Margin 9100265 | 16.7 |
| (ODO4286) | | | |
| Muscle Margin (ODO4286) | 47.6 | Breast Cancer A209073 | 38.2 |
| Lung Malignant Cancer | 31.9 | Breast Margin | 44.8 |
| (OD03126) | | A2090734 | |
| Lung Margin (OD03126) | 64.2 | Normal Liver | 23.2 |
| Lung Cancer (OD04404) | 58.6 | Liver Cancer | 23.3 |
| Lung Margin (OD04404) | 38.2 | Liver Cancer 1025 | 10.5 |
| Lung Cancer (OD04565) | 15.8 | Liver Cancer 1026 | 6.7 |
| Lung Margin (OD04565) | 26.4 | Liver Cancer 6004-T | 14.1 |
| Lung Cancer (OD04237-01) | 37.6 | Liver Tissue 6004-N | 9.5 |
| Lung Margin (OD04237-02) | 48.0 | Liver Cancer 6005-T | 6.7 |
| Ocular Mel Met to Liver | 14.9 | Liver Tissue 6005-N | 6.7 |
| (ODO4310) | | | |
| Liver Margin (ODO4310) | 13.5 | Normal Bladder | 49.0 |
| Melanoma Metastasis | 36.6 | Bladder Cancer | 5.7 |
| Lung Margin (OD04321) | 50.3 | Bladder Cancer | 32.5 |
| Normal Kidney | 84.7 | Bladder Cancer | 52.1 |
| | | (OD04718-01) | |
| Kidney Ca, Nuclear grade 2 | 65.1 | Bladder Normal | 63.7 |
| (OD04338) | | Adjacent (OD04718-03) | |
| Kidney Margin (OD04338) | 46.3 | Normal Ovary | 6.0 |
| Kidney Ca Nuclear grade | 33.4 | Ovarian Cancer | 63.3 |
| 1/2 (OD04339) | | | |
| Kidney Margin (OD04339) | 77.9 | Ovarian Cancer | 43.8 |
| | | (OD04768-07) | |
| Kidney Ca, Clear cell type | 71.7 | Ovary Margin | 14.6 |
| (OD04340) | | (OD04768-08) | |
| Kidney Margin (OD04340) | 57.0 | Normal Stomach | 30.4 |
| Kidney Ca, Nuclear grade 3 | 17.2 | Gastric Cancer 9060358 | 10.4 |
| (OD04348) | | | |
| Kidney Margin (OD04348) | 28.9 | Stomach Margin | 12.9 |
| | | 9060359 | |
| Kidney Cancer (OD04622- | 21.9 | Gastric Cancer 9060395 | 56.3 |
| 01) | | | |
| Kidney Margin (OD04622- | 4.3 | Stomach Margin | 30.4 |
| 03) | | 9060394 | |
| Kidney Cancer (OD04450- | 29.5 | Gastric Cancer 9060397 | 33.2 |
| 01) | | | |
| Kidney Margin (OD04450- | 36.9 | Stomach Margin | 8.9 |
| 03) | | 9060396 | |
| Kidney Cancer 8120607 | 3.4 | Gastric Cancer 064005 | 53.6 |

TABLE 35
Panel 4D

| | Rel. Exp. (%) | | Rel. Exp. (%) |
|---|---|---|---|
| | Ag2304, Run | | Ag2304, Run |
| Tissue Name | 159131012 | Tissue Name | 159131012 |
| Secondary Th1 act | 32.5 | HUVEC IL-1beta | 5.8 |
| Secondary Th2 act | 46.7 | HUVEC IFN gamma | 19.6 |
| Secondary Tr1 act | 47.0 | HUVEC TNF alpha + IFN | 12.2 |
| | | gamma | |
| Secondary Th1 rest | 14.5 | HUVEC TNF alpha + IL4 | 9.8 |
| Secondary Th2 rest | 27.0 | HUVEC IL-11 | 8.8 |
| Secondary Tr1 rest | 23.5 | Lung Microvascular EC none | 5.8 |
| Primary Th1 act | 44.1 | Lung Microvascular EC | 12.8 |
| | | TNF alpha + IL-1beta | |
| Primary Th2 act | 39.2 | Microvascular Dermal EC | 20.6 |
| | | none | |
| Primary Tr1 act | 49.3 | Microsvasular Dermal EC | 16.0 |
| | | TNF alpha + IL-1beta | |
| Primary Th1 rest | 95.3 | Bronchial epithelium | 14.2 |
| | | TNF alpha + IL1beta | |
| Primary Th2 rest | 54.7 | Small airway epithelium | 7.9 |
| | | none | |
| Primary Tr1 rest | 29.5 | Small airway epithelium | 38.4 |
| | | TNF alpha + IL-1beta | |
| CD45RA CD4 | 21.5 | Coronery artery SMC rest | 25.3 |
| lymphocyte act | | | |
| CD45RO CD4 | 37.1 | Coronery artery SMC | 12.7 |
| lymphocyte act | | TNF alpha + IL-1beta | |
| CD8 lymphocyte act | 20.9 | Astrocytes rest | 23.0 |
| Secondary CD8 | 29.1 | Astrocytes TNF alpha + IL- | 23.7 |
| lymphocyte rest | | 1beta | |
| Secondary CD8 | 22.7 | KU-812 (Basophil) rest | 4.6 |
| lymphocyte act | | | |
| CD4 lymphocyte none | 26.6 | KU-812 (Basophil) | 11.0 |
| | | PMA/ionomycin | |
| 2ry Th1/Th2/Tr1_anti- | 34.2 | CCD1106 (Keratinocytes) | 15.8 |
| CD95 CH11 | | none | |
| LAK cells rest | 41.5 | CCD1106 (Keratinocytes) | 5.1 |
| | | TNF alpha + IL-1beta | |
| LAK cells IL-2 | 33.2 | Liver cirrhosis | 2.8 |
| LAK cells IL-2 + IL-12 | 22.8 | Lupus kidney | 4.3 |
| LAK cells IL-2 + IFN | 36.9 | NCI-H292 none | 34.9 |
| gamma | | | |
| LAK cells IL-2 + IL-18 | 38.4 | NCI-H292 IL-4 | 38.4 |
| LAK cells | 34.2 | NCI-H292 IL-9 | 39.2 |
| PMA/ionomycin | | | |
| NK Cells IL-2 rest | 26.6 | NCI-H292 IL-13 | 21.2 |
| Two Way MLR 3 day | 53.2 | NCI-H292 IFN gamma | 20.9 |
| Two Way MLR 5 day | 26.2 | HPAEC none | 11.3 |
| Two Way MLR 7 day | 13.3 | HPAEC TNF alpha + IL- | 11.7 |
| | | 1beta | |
| PBMC rest | 14.5 | Lung fibroblast none | 18.9 |
| PBMC PWM | 83.5 | Lung fibroblast TNF alpha + | 22.8 |
| | | IL-1beta | |
| PBMC PHA-L | 31.9 | Lung fibroblast IL-4 | 25.9 |
| Ramos (B cell) none | 11.4 | Lung fibroblast IL-9 | 13.4 |
| Ramos (B cell) | 34.4 | Lung fibroblast IL-13 | 18.9 |
| ionomycin | | | |
| B lymphocytes PWM | 60.3 | Lung fibroblast IFN gamma | 46.0 |
| B lymphocytes CD40L | 16.6 | Dermal fibroblast CCD1070 | 47.0 |
| and IL-4 | | rest | |
| EOL-1 dbcAMP | 34.2 | Dermal fibroblast CCD1070 | 83.5 |
| | | TNF alpha | |
| EOL-1 dbcAMP | 100.0 | Dermal fibroblast CCD1070 | 23.7 |
| PMA/ionomycin | | IL-1beta | |
| Dendritic cells none | 13.7 | Dermal fibroblast IFN | 18.3 |
| | | gamma | |
| Dendritic cells LPS | 16.5 | Dermal fibroblast IL-4 | 25.2 |
| Dendritic cells anti- | 6.3 | IBD Colitis 2 | 2.3 |
| CD40 | | | |
| Monocytes rest | 23.5 | IBD Crohn's | 4.3 |
| Monocytes LPS | 84.1 | Colon | 24.5 |
| Macrophages rest | 23.3 | Lung | 23.7 |
| Macrophages LPS | 31.4 | Thymus | 39.0 |
| HUVEC none | 15.0 | Kidney | 44.4 |
| HUVEC starved | 30.6 | | |

CNS_neurodegeneration_v1.0 Summary: Ag2304 Expression of the NOV3 gene in this panel is ubiquitous. While this gene does not show differential expression between Alzheimer's diseased brains and control brains, this panel confirms the expression of this gene in the brains of an independent group of patients. See Panel 1.3d for utility of this gene in the central nervous system.

Panel 1.3D Summary: Ag2304 The NOV3 gene, a homolog of the Drosophila pecanex gene, is widely expressed across the samples in this panel, with highest expression in the hippocampus (CT=28.6). In addition, this gene is expressed at moderate to high levels in all CNS regions examined. Expression of this gene in both the mother and developing embryo is critical for normal CNS development. Furthermore, expression of this protein appears to be involved in stem cell fate determination, where removal of this protein increases neural precursor cells. Therefore, downregulation of this gene could be used in neural stem cell research and therapy to control the fate of stem cells and increasing the resulting numbers of post-mitotic neurons.

The NOV3 gene is modestly expressed in a wide variety of metabolic tissues including adipose, adrenal, pancreas, thyroid, pituitary, heart, adult and fetal skeletal muscle, and adult and fetal liver. This widespread expression in tissues with metabolic function suggests that the NOV3 gene product may be important for the pathogenesis, diagnosis, and/or treatment of metabolic disease in any or all of these tissues, including obesity and diabetes.

References:

1. LaBonne S G, Furst A. Differentiation in vitro of neural precursor cells from normal and Pecanex mutant Drosophila embryos. J Neurogenet May 5, 1989; (2):99-104

Early gastrula embryos, lacking both maternally and zygotically expressed activity of the neurogenic pecanex locus, are shown to contain a greater than wild-type number of stably determined neural precursor cells which can differentiate into neurons in culture.

2. LaBonne S G, Sunitha I, Mahowald A P. Molecular genetics of pecanex, a maternal- effect neurogenic locus of Drosophila melanogaster that potentially encodes a large transmembrane protein. Dev Biol November 1989; 136(1):1-16

In the absence of maternal expression of the pecanex gene, the embryo develops severe hyperneuralization similar to that characteristic of Notch mutant embryos. We have extended a previous molecular analysis of the chromosomal interval that encompasses pecanex by using additional deficiencies to localize the locus on the molecular map. RNA blot analysis shows that the locus encodes a rare 9-kb transcript as well as minor transcripts of 3.7 and 2.3 kb. The temporal expression of these transcripts is appropriate for a neurogenic locus. Phenocopies of the mutant phenotype have been produced following microinjection of antisense RNA corresponding to a portion of the pecanex transcripts. Conceptual translation of a partial coding sequence compiled from cDNA and genomic clones indicates that the pecanex locus potentially encodes a large, membrane-spanning protein.

Panel 2D Summary: Ag2304 The expression of this gene appears to be highest in a sample derived from normal lung tissue. Thus, the expression of this gene could be used to distinguish normal lung tissue from other tissues in the panel. Of note is the difference in expression between samples derived from ovarian cancer and normal adjacent tissue. This difference in levels of expression is also notable in samples derived from gastric cancer when compared to their normal counterparts. Thus, the expression of this gene could be used to distinguish ovarian or gastric cancer form their normal adjacent tissues. Moreover, therapeutic modulation of this gene, through the use of small molecule drugs, antibodies or protein therapeutics might be of use in the treatment of ovarian or gastric cancer.

Panel 4D Summary: Ag 2304 This NOV3 transcript is detected ubiquitously throughout this panel, with highest expression of this transcript in activated eosinophils (CT=28.1). This indicates an up-regulation of this transcript in these cells upon activation. Eosinophils contribute to the pathology of several atopic diseases such as asthma, atopic dermatitis, and rhinitis. Therefore, modulation of the activity or activation of the protein encoded by the NOV3 gene may be beneficial for the treatment of those diseases. The NOV3 gene is also highly expressed in effector T cells, activated monocytes and dermal fibroblasts upon treatment with TNF-a and IL-1b. Modulation of the expression of this transcript, which encodes for a Pecanex like molecule, could be beneficial in the treatment of inflammatory diseases associated with T cell activation as well as eosinophil activation including atopic diseases and autoimmune diseases such as rheumatoid arthritis, inflammatory bowel disease and skin inflammation.

NOV4

Expression of gene NOV4 was assessed using the primer-probe set Ag2428, described in Table 36. Results of the RTQ-PCR runs are shown in Tables 37, 38, 39 and 40.

TABLE 36

Probe Name Ag2428

| | | | Start | SEQ ID |
|---|---|---|---|---|
| Primers | Sequence | Length | Position | NO: |
| Forward | 5′-gagccagggctgctgtata-3′ | 19 | 1419 | 154 |
| Probe | TET-5′-cctctcaggaacatgctaccaaaatt-3′-TAMRA | 26 | 1439 | 155 |
| Reverse | 5′-tagattgagggcagcagtca-3′ | 20 | 1476 | 156 |

TABLE 37
CNS_neurodegeneration_v1.0

| | Rel. | | Rel. |
|---|---|---|---|
| | Exp. (%) | | Exp. (%) |
| | Ag2428, | | Ag2428, |
| | Run | | Run |
| Tissue Name | 206271177 | Tissue Name | 206271177 |
| AD 1 Hippo | 7.9 | Control (Path) 3 | 3.5 |
| | | Temporal Ctx | |
| AD 2 Hippo | 22.2 | Control (Path) 4 | 40.3 |
| | | Temporal Ctx | |
| AD 3 Hippo | 12.8 | AD 1 Occipital Ctx | 18.3 |
| AD 4 Hippo | 5.1 | AD 2 Occipital Ctx | 0.0 |
| | | (Missing) | |
| AD 5 Hippo | 100.0 | AD 3 Occipital Ctx | 7.0 |
| AD 6 Hippo | 32.5 | AD 4 Occipital Ctx | 20.3 |
| Control 2 Hippo | 10.9 | AD 5 Occipital Ctx | 25.0 |
| Control 4 Hippo | 17.0 | AD 5 Occipital Ctx | 16.5 |
| Control (Path) 3 | 6.7 | Control 1 Occipital | 6.0 |
| Hippo | | Ctx | |
| AD 1 Temporal Ctx | 16.6 | Control 2 Occipital | 21.0 |
| | | Ctx | |
| AD 2 Temporal Ctx | 23.2 | Control 3 Occipital | 23.2 |
| | | Ctx | |
| AD 3 Temporal Ctx | 9.4 | Control 4 Occipital | 6.0 |
| | | Ctx | |
| AD 4 Temporal Ctx | 25.9 | Control (Path) 1 | 50.3 |
| | | Occipital Ctx | |
| AD 5 Inf Temporal | 40.1 | Control (Path) 2 | 13.2 |
| Ctx | | Occipital Ctx | |
| AD 5 Sup Temporal | 33.7 | Control (Path) 3 | 1.1 |
| Ctx | | Occipital Ctx | |
| AD 6 Inf Temporal | 35.6 | Control (Path) 4 | 30.4 |
| Ctx | | Occipital Ctx | |
| AD 6 Sup Temporal | 48.3 | Control 1 Parietal Ctx | 12.4 |
| Ctx | | | |
| Control 1 Temporal | 8.0 | Control 2 Parietal Ctx | 46.0 |
| Ctx | | | |
| Control 2 Temporal | 8.5 | Control 3 Parietal Ctx | 23.7 |
| Ctx | | | |
| Control 3 Temporal | 14.7 | Control (Path) 1 | 38.7 |
| Ctx | | Parietal Ctx | |
| Control 3 Temporal | 11.3 | Control (Path) 2 | 20.4 |
| Ctx | | Parietal Ctx | |
| Control (Path) 1 | 37.6 | Control (Path) 3 | 5.4 |
| Temporal Ctx | | Parietal Ctx | |
| Control (Path) 2 | 34.9 | Control (path) 4 | 43.2 |
| Temporal Ctx | | Parietal Ctx | |

TABLE 38
Panel 1.3D

| | Rel. Exp. (%) | | Rel. Exp. (%) |
|---|---|---|---|
| | Ag2428, | | Ag2428, |
| Tissue Name | Run 159361380 | Tissue Name | Run 159361380 |
| Liver adenocarcinoma | 10.7 | Kidney (fetal) | 7.1 |
| Pancreas | 2.4 | Renal ca. 786-0 | 6.6 |
| Pancreatic ca. CAPAN 2 | 6.0 | Renal ca. A498 | 18.8 |
| Adrenal gland | 5.3 | Renal ca. RXF 393 | 3.8 |
| Thyroid | 2.5 | Renal ca. ACHN | 1.1 |
| Salivary gland | 3.7 | Renal ca. UO-31 | 4.5 |
| Pituitary gland | 7.2 | Renal ca. TK-10 | 5.1 |
| Brain (fetal) | 5.2 | Liver | 1.9 |
| Brain (whole) | 5.1 | Liver (fetal) | 11.4 |
| Brain (amygdala) | 5.1 | Liver ca. (hepatoblast) | 8.0 |
| | | HepG2 | |
| Brain (cerebellum) | 2.7 | Lung | 8.5 |
| Brain (hippocampus) | 17.6 | Lung (fetal) | 4.7 |
| Brain (substantia nigra) | 1.8 | Lung ca. (small cell) | 7.5 |
| | | LX-1 | |
| Brain (thalamus) | 4.9 | Lung ca. (small cell) | 11.9 |
| | | NCI-H69 | |
| Cerebral Cortex | 2.8 | Lung ca. (s. cell var.) | 25.2 |
| | | SHP-77 | |
| Spinal cord | 3.8 | Lung ca. (large | 8.8 |
| | | cell)NCI-H460 | |
| glio/astro U87-MG | 12.9 | Lung ca. (non-sm. cell) | 8.3 |
| | | A549 | |
| glio/astro U-118-MG | 39.5 | Lung ca. (non-s. cell) | 18.3 |
| | | NCI-H23 | |
| astrocytoma SW1783 | 5.4 | Lung ca. (non-s. cell) | 6.6 |
| | | HOP-62 | |
| neuro*; met SK-N-AS | 100.0 | Lung ca. (non-s. cl) | 8.4 |
| | | NCI-H522 | |
| astrocytoma SF-539 | 7.6 | Lung ca. (squam.) SW | 9.7 |
| | | 900 | |
| astrocytoma SNB-75 | 19.8 | Lung ca. (squam.) | 5.4 |
| | | NCI-H596 | |
| glioma SNB-19 | 12.0 | Mammary gland | 5.9 |
| glioma U251 | 11.3 | Breast ca.* (pl. ef) | 10.9 |
| | | MCF-7 | |
| glioma SF-295 | 7.4 | Breast ca.* (pl. ef) | 66.0 |
| | | MDA-MB-231 | |
| Heart (Fetal) | 2.0 | Breast ca.* (pl. ef) | 9.6 |
| | | T47D | |
| Heart | 5.1 | Breast ca. BT-549 | 34.4 |
| Skeletal muscle (Fetal) | 8.5 | Breast ca. MDA-N | 17.7 |
| Skeletal muscle | 1.2 | Ovary | 2.1 |
| Bone marrow | 17.8 | Ovarian ca. OVCAR-3 | 10.8 |
| Thymus | 5.6 | Ovarian ca. OVCAR-4 | 0.6 |
| Spleen | 10.1 | Ovarian ca. OVCAR-5 | 5.6 |
| Lymph node | 9.2 | Ovarian ca. OVCAR-8 | 10.0 |
| Colorectal | 6.9 | Ovarian ca. IGROV-1 | 2.1 |
| Stomach | 7.3 | Ovarian ca. (ascites) | 15.1 |
| | | SK-OV-3 | |
| Small intestine | 8.1 | Uterus | 3.7 |
| Colon ca. SW480 | 8.5 | Placenta | 3.8 |
| Colon ca.* SW620 | 13.6 | Prostate | 6.0 |
| (SW480 met) | | | |
| Colon ca. HT29 | 11.0 | Prostate ca.* (bone | 5.8 |
| | | met) PC-3 | |
| Colon ca. HCT-116 | 12.9 | Testis | 9.3 |
| Colon ca. CaCo-2 | 12.3 | Melanoma Hs688(A).T | 2.6 |
| CC Well to Mod Diff | 7.6 | Melanoma* (met) | 1.9 |
| (ODO3866) | | Hs688(B).T | |
| Colon ca. HCC-2998 | 33.0 | Melanoma UACC-62 | 3.8 |
| Gastric ca. (liver met) | 25.9 | Melanoma M14 | 5.8 |
| NCI-N87 | | | |
| Bladder | 10.2 | Melanoma LOX IMVI | 5.2 |
| Trachea | 9.0 | Melanoma* (met) SK- | 10.3 |
| | | MEL-5 | |
| Kidney | 2.5 | Adipose | 6.3 |

TABLE 39
Panel 2D

| | Rel. Exp. (%) | | Rel. Exp. (%) |
|---|---|---|---|
| | Ag2428, | | Ag2428, |
| Tissue Name | Run 159361727 | Tissue Name | Run 159361727 |
| Normal Colon | 80.1 | Kidney Margin | 2.1 |
| | | 8120608 | |
| CC Well to Mod Diff | 13.7 | Kidney Cancer 8120613 | 15.3 |
| (ODO3866) | | | |
| CC Margin (ODO3866) | 7.7 | Kidney Margin | 6.8 |
| | | 8120614 | |
| CC Gr.2 rectosigmoid | 25.5 | Kidney Cancer 9010320 | 21.3 |
| (ODO3868) | | | |
| CC Margin (ODO3868) | 6.6 | Kidney Margin | 17.4 |
| | | 9010321 | |
| CC Mod Diff (ODO3920) | 70.2 | Normal Uterus | 3.2 |
| CC Margin (ODO3920) | 30.4 | Uterine Cancer 064011 | 21.3 |
| CC Gr.2 ascend colon | 52.1 | Normal Thyroid | 9.9 |
| (ODO3921) | | | |
| CC Margin (ODO3921) | 11.1 | Thyroid Cancer | 5.8 |
| CC from Partial | 50.7 | Thyroid Cancer | 27.7 |
| Hepatectomy (ODO4309) | | A302152 | |
| Mets | | | |
| Liver Margin (ODO4309) | 22.7 | Thyroid Margin | 37.6 |
| | | A302153 | |
| Colon mets to lung | 29.1 | Normal Breast | 18.7 |
| (OD04451-01) | | | |
| Lung Margin (OD04451-02) | 7.0 | Breast Cancer | 26.4 |
| Normal Prostate 6546-1 | 8.8 | Breast Cancer | 75.3 |
| | | (OD04590-01) | |
| Prostate Cancer (OD04410) | 49.3 | Breast Cancer Mets | 87.1 |
| | | (OD04590-03) | |
| Prostate Margin (OD04410) | 41.2 | Breast Cancer | 48.0 |
| | | Metastasis | |
| Prostate Cancer (OD04720- | 52.9 | Breast Cancer | 49.7 |
| 01) | | | |
| Prostate Margin (OD04720- | 59.5 | Breast Cancer | 36.3 |
| 02) | | | |
| Normal Lung | 81.8 | Breast Cancer 9100266 | 18.4 |
| Lung Met to Muscle | 30.1 | Breast Margin 9100265 | 14.9 |
| (ODO4286) | | | |
| Muscle Margin (ODO4286) | 13.9 | Breast Cancer A209073 | 55.5 |
| Lung Malignant Cancer | 47.3 | Breast Margin | 45.1 |
| (OD03126) | | A2090734 | |
| Lung Margin (OD03126) | 41.8 | Normal Liver | 15.8 |
| Lung Cancer (OD04404) | 28.5 | Liver Cancer | 14.4 |
| Lung Margin (OD04404) | 16.7 | Liver Cancer 1025 | 5.7 |
| Lung Cancer (OD04565) | 28.3 | Liver Cancer 1026 | 5.7 |
| Lung Margin (OD04565) | 14.0 | Liver Cancer 6004-T | 7.9 |
| Lung Cancer (OD04237-01) | 62.4 | Liver Tissue 6004-N | 8.1 |
| Lung Margin (OD04237-02) | 28.3 | Liver Cancer 6005-T | 5.2 |
| Ocular Mel Met to Liver | 23.5 | Liver Tissue 6005-N | 0.8 |
| (ODO4310) | | | |
| Liver Margin (ODO4310) | 11.3 | Normal Bladder | 81.8 |
| Melanoma Metastasis | 40.9 | Bladder Cancer | 9.2 |
| Lung Margin (OD04321) | 26.2 | Bladder Cancer | 62.0 |
| Normal Kidney | 54.3 | Bladder Cancer | 32.8 |
| | | (OD04718-01) | |
| Kidney Ca, Nuclear grade 2 | 40.1 | Bladder Normal | 24.8 |
| (OD04338) | | Adjacent (OD04718-03) | |
| Kidney Margin (OD04338) | 45.7 | Normal Ovary | 0.8 |
| Kidney Ca Nuclear grade | 82.9 | Ovarian Cancer | 51.8 |
| 1/2 (OD04339) | | | |
| Kidney Margin (OD04339) | 45.4 | Ovarian Cancer | 86.5 |
| | | (OD04768-07) | |
| Kidney Ca, Clear cell type | 49.3 | Ovary Margin | 8.5 |
| (OD04340) | | (OD04768-08) | |
| Kidney Margin (OD04340) | 48.0 | Normal Stomach | 20.7 |
| Kidney Ca, Nuclear grade 3 | 24.3 | Gastric Cancer 9060358 | 6.9 |
| (OD04348) | | | |
| Kidney Margin (OD04348) | 40.3 | Stomach Margin | 13.1 |
| | | 9060359 | |
| Kidney Cancer (OD04622- | 10.7 | Gastric Cancer 9060395 | 23.5 |
| 01) | | | |
| Kidney Margin (OD04622- | 3.1 | Stomach Margin | 18.9 |
| 03) | | 9060394 | |
| Kidney Cancer (OD04450- | 24.8 | Gastric Cancer 9060397 | 39.8 |
| 01) | | | |
| Kidney Margin (OD04450- | 25.5 | Stomach Margin | 7.1 |
| 03) | | 9060396 | |
| Kidney Cancer 8120607 | 2.7 | Gastric Cancer 064005 | 100.0 |

TABLE 40
Panel 4D

| | Rel. Exp. (%) | | Rel. Exp. (%) |
|---|---|---|---|
| | Ag2428, Run | | Ag2428, Run |
| Tissue Name | 159362614 | Tissue Name | 159362614 |
| Secondary Th1 act | 26.8 | HUVEC IL-1beta | 9.2 |
| Secondary Th2 act | 34.6 | HUVEC IFN gamma | 14.1 |
| Secondary Tr1 act | 37.6 | HUVEC TNF alpha + IFN | 8.4 |
| | | gamma | |
| Secondary Th1 rest | 10.7 | HUVEC TNF alpha + IL4 | 12.0 |
| Secondary Th2 rest | 13.6 | HUVEC IL-11 | 7.9 |
| Secondary Tr1 rest | 16.7 | Lung Microvascular EC none | 10.9 |
| Primary Th1 act | 36.9 | Lung Microvascular EC | 9.9 |
| | | TNF alpha + IL-1beta | |
| Primary Th2 act | 48.3 | Microvascular Dermal EC | 17.0 |
| | | none | |
| Primary Tr1 act | 50.7 | Microsvasular Dermal EC | 10.6 |
| | | TNF alpha + IL-1beta | |
| Primary Th1 rest | 74.2 | Bronchial epithelium | 9.8 |
| | | TNF alpha + IL-1beta | |
| Primary Th2 rest | 41.5 | Small airway epithelium | 3.6 |
| | | none | |
| Primary Tr1 rest | 28.9 | Small airway epithelium | 38.7 |
| | | TNF alpha + IL-1beta | |
| CD45RA CD4 | 22.7 | Coronery artery SMC rest | 9.9 |
| lymphocyte act | | | |
| CD45RO CD4 | 31.0 | Coronery artery SMC | 4.2 |
| lymphocyte act | | TNF alpha + IL-1beta | |
| CD8 lymphocyte act | 15.9 | Astrocytes rest | 5.9 |
| Secondary CD8 | 19.6 | Astrocytes TNF alpha + IL- | 5.8 |
| lymphocyte rest | | 1beta | |
| Secondary CD8 | 17.9 | KU-812 (Basophil) rest | 8.7 |
| lymphocyte act | | | |
| CD4 lymphocyte none | 11.6 | KU-812 (Basophil) | 31.6 |
| | | PMA/ionomycin | |
| 2ry Th1/Th2/Tr1_anti- | 18.8 | CCD1106 (Keratinocytes) | 12.3 |
| CD95 CH11 | | none | |
| LAK cells rest | 20.4 | CCD1106 (Keratinocytes) | 8.4 |
| | | TNF alpha + IL-1beta | |
| LAK cells IL-2 | 27.7 | Liver cirrhosis | 4.7 |
| LAK cells IL-2 + IL-12 | 20.0 | Lupus kidney | 2.1 |
| LAK cells IL-2 + IFN | 38.4 | NCI-H292 none | 25.5 |
| gamma | | | |
| LAK cells IL-2 + IL-18 | 43.5 | NCI-H292 IL-4 | 37.1 |
| LAK cells | 13.6 | NCI-H292 IL-9 | 36.9 |
| PMA/ionomycin | | | |
| NK Cells IL-2 rest | 21.2 | NCI-H292 IL-13 | 15.6 |
| Two Way MLR 3 day | 23.8 | NCI-H292 IFN gamma | 14.1 |
| Two Way MLR 5 day | 11.3 | HPAEC none | 11.1 |
| Two Way MLR 7 day | 11.3 | HPAEC TNF alpha + IL- | 13.5 |
| | | 1beta | |
| PBMC rest | 7.9 | Lung fibroblast none | 10.7 |
| PBMC PWM | 60.3 | Lung fibroblast TNF alpha + | 5.0 |
| | | IL-1beta | |
| PBMC PHA-L | 23.5 | Lung fibroblast IL-4 | 18.6 |
| Ramos (B cell) none | 23.5 | Lung fibroblast IL-9 | 13.0 |
| Ramos (B cell) | 80.7 | Lung fibroblast IL-13 | 11.0 |
| ionomycin | | | |
| B lymphocytes PWM | 100.0 | Lung fibroblast IFN gamma | 13.6 |
| B lymphocytes CD40L | 44.8 | Dermal fibroblast CCD1070 | 27.9 |
| and IL-4 | | rest | |
| EOL-1 dbcAMP | 11.0 | Dermal fibroblast CCD1070 | 82.9 |
| | | TNF alpha | |
| EOL-1 dbcAMP | 19.6 | Dermal fibroblast CCD1070 | 15.0 |
| PMA/ionomycin | | IL-1beta | |
| Dendritic cells none | 9.5 | Dermal fibroblast IFN | 10.1 |
| | | gamma | |
| Dendritic cells LPS | 7.6 | Dermal fibroblast IL-4 | 11.9 |
| Dendritic cells anti- | 5.5 | IBD Colitis 2 | 2.8 |
| CD40 | | | |
| Monocytes rest | 15.8 | IBD Crohn's | 2.2 |
| Monocytes LPS | 11.0 | Colon | 11.0 |
| Macrophages rest | 9.3 | Lung | 5.3 |
| Macrophages LPS | 5.2 | Thymus | 18.6 |
| HUVEC none | 12.2 | Kidney | 41.8 |
| HUVEC starved | 33.0 | | |

CNS_neurodegeneration_v1.0 Summary: Ag2428 While results from this experiment show that this gene is not differentially expressed in the Alzheimer's diseased brain, this panel confirms the expression of this gene at moderate levels in the CNS in an independent group of patients. Please see Panel 1.3D for a discussion of utility of this gene in the central nervous system.

Panel 1.3D Summary: Ag2428 The NOV4 gene is expressed widely across many samples in this panel, with highest expression in a sample derived from a neuroblastoma cell line(CT=29.8). Moreover, there appears to be a cluster of expression associated with breast cancer cell lines. Thus, the expression of this gene could be used to distinguish these samples from others in the panel.

In addition, the NOV4 gene is moderately expressed in a number of metabolic tissues including adipose, adrenal, pituitary, heart, fetal skeletal muscle and fetal liver. Thus, this gene product may be an important small molecule target for the treatment of metabolic disease, including obesity and Type 2 diabetes.

This gene is expressed at low levels in the CNS, and is an an aurora-related kinase. The aurora-related kinases are involed in the control of the cell-cycle, and may be useful in the control of cell fate in neural stem cells. This protein may therefore be of use in stem cell research or therapy.

References:

Severson A F, Hamill D R, Carter J C, Schumacher J, Bowerman B. The aurora-related kinase AIR-2 recruits ZEN-4/CeMKLP1 to the mitotic spindle at metaphase and is required for cytokinesis. Curr Biol October 2000 5; 10(19):1162-71

BACKGROUND: The Aurora/Ipl1p-related kinase AIR-2 is required for mitotic chromosome segregation and cytokinesis in early Caenorhabditis elegans embryos. Previous studies have relied on non-conditional mutations or RNA-mediated interference (RNAi) to inactivate AIR-2. It has therefore not been possible to determine whether AIR-2 functions directly in cytokinesis or if the cleavage defect results indirectly from the failure to segregate DNA. One intriguing hypothesis is that AIR-2 acts to localize the mitotic kinesin-like protein ZEN-4 (also known as CeMKLP1), which later functions in cytokinesis. RESULTS: Using conditional alleles, we established that AIR-2 is required at metaphase or early anaphase for normal segregation of chromosomes, localization of ZEN-4, and cytokinesis. ZEN-4 is first required late in cytokinesis, and also functions to maintain cell separation through much of the subsequent interphase. DNA segregation defects alone were not sufficient to disrupt cytokinesis in other mutants, suggesting that AIR-2 acts specifically during cytokinesis through ZEN-4. AIR-2 and ZEN-4 shared similar genetic interactions with the formin homology (FH) protein CYK-1, suggesting that AIR-2 and ZEN-4 function in a single pathway, in parallel to a contractile ring pathway that includes CYK-1. Using in vitro co-immunoprecipitation experiments, we found that AIR-2 and ZEN-4 interact directly. CONCLUSIONS: AIR-2 has two functions during mitosis: one in chromosome segregation, and a second, independent function in cytokinesis through ZEN-4. AIR-2 and ZEN-4 may act in parallel to a second pathway that includes CYK-1.

Panel 2D Summary: Ag2428 The expression of this gene is found widely across a number of samples in this panel. It is found to be highest in a sample derived from a gastric cancer. Of note is the association observed between gastric cancer samples, when compared to their normal adjacent samples. This association is also notable in ovarian cancer and breast cancer. Thus, the expression of this gene could be used to distinguish gastric cancer, breast cancer and ovarian cancer from their normal adjacent tissues. Morover, therapeutic modulation of this gene, through the use of small molecule drugs, antibodies or protein therapeutics might be of benefit in the treatment of gastric, breast or ovarian cancer.

Panel 4D Summary: Ag 2428 This transcript is ubiquitously expressed in all cells throughout the panel. However, the highest expression of this transcript is found in B cells upon activation with the B cell mitogen, PWM. Significant expression of this transcript in the activated Ramos B cell line is consistent with this finding. This transcript encodes an aurora- related kinase 1 which belongs to a family of oncogenic mitogenic serine threonine kinases (see reference below). Therefore, modulation of the expression of this transcript by small molecules, may be beneficial for the treatment of diaseases associated with hyperproliferation of B cells including B cell lymphomas, hyperglobulinemia and autoimmune disease such as lupus and rheumatoid arthritis. This transcript is also expressed in dermal fibroblasts upon treatment with TNF-a and Il-1 and in primary Th1 cells suggesting that modulation of this transcript may be important in the treatment of T cell mediated diseases and inflammatory skin diseases.

Reference:

1. J Cell Sci November 1999; 112 (Pt 21):3591-601. Aurora/Ipl1p-related kinases, a new oncogenic family of mitotic serine-threonine kinases. Giet R, Prigent C.

CNRS UPR41| Universite de Rennes I, Groupe Cycle Cellulaire, Faculte de Medecine, CS 34317, France.

During the past five years, a growing number of serine-threonine kinases highly homologous to the Saccharomyces cerevisiae Ipl1p kinase have been isolated in various organisms. A Drosophila melanogaster homologue, aurora, was the first to be isolated from a multicellular organism. Since then, several related kinases have been found in mammalian cells. They localise to the mitotic apparatus: in the centrosome, at the poles of the bipolar spindle or in the midbody. The kinases are necessary for completion of mitotic events such as centrosome separation, bipolar spindle assembly and chromosome segregation. Extensive research is now focusing on these proteins because the three human homologues are overexpressed in various primary cancers. Furthermore, overexpression of one of these kinases transforms cells. Because of the myriad of kinases identified, we suggest a generic name: Aurora/Ipl1p-related kinase (AIRK). We denote AIRKs with a species prefix and a number, e.g. HsAIRK1.

NOV5

Expression of gene NOV5 was assessed using the primer-probe set Ag2423, described in Table 41. Results of the RTQ-PCR runs are shown in Tables 42, 43 and 44.

TABLE 41

Probe Name Ag2423

| | | | Start | SEQ ID |
|---|---|---|---|---|
| Primers | Sequences | Length | Position | NO: |
| Forward | 5′-aactgccactggtgacactt-3′ | 20 | 243 | 157 |
| Probe | TET-5′-cacactcagtgtcggttaaaattactga-3′-TAMRA | 28 | 263 | 158 |
| Reverse | 5′-tgaattcttccaccatgagaa-3′ | 21 | 315 | 159 |

TABLE 42
Panel 1.3D

| | Rel. Exp. (%) | | Rel. Exp. (%) |
|---|---|---|---|
| | Ag2423, | | Ag2423, |
| Tissue Name | Run 159337657 | Tissue Name | Run 159337657 |
| Liver adenocarcinoma | 25.9 | Kidney (fetal) | 0.0 |
| Pancreas | 8.4 | Renal ca. 786-0 | 11.7 |
| Pancreatic ca. CAPAN 2 | 7.1 | Renal ca. A498 | 7.2 |
| Adrenal gland | 65.1 | Renal ca. RXF 393 | 9.6 |
| Thyroid | 4.0 | Renal ca. ACHN | 8.5 |
| Salivary gland | 41.8 | Renal ca. UO-31 | 8.4 |
| Pituitary gland | 9.9 | Renal ca. TK-10 | 4.5 |
| Brain (fetal) | 75.8 | Liver | 13.6 |
| Brain (whole) | 5.1 | Liver (fetal) | 28.1 |
| Brain (amygdala) | 5.8 | Liver ca. (hepatoblast) | 12.9 |
| | | HepG2 | |
| Brain (cerebellum) | 3.8 | Lung | 21.0 |
| Brain (hippocampus) | 66.4 | Lung (fetal) | 15.4 |
| Brain (substantia nigra) | 20.4 | Lung ca. (small cell) | 4.9 |
| | | LX-1 | |
| Brain (thalamus) | 7.4 | Lung ca. (small cell) | 4.6 |
| | | NCI-H69 | |
| Cerebral Cortex | 52.9 | Lung ca. (s. cell var.) | 14.7 |
| | | SHP-77 | |
| Spinal cord | 22.5 | Lung ca. (large | 15.7 |
| | | cell)NCI-H460 | |
| glio/astro U87-MG | 11.5 | Lung ca. (non-sm. cell) | 9.8 |
| | | A549 | |
| glio/astro U-118-MG | 11.1 | Lung ca. (non-s. cell) | 12.2 |
| | | NCI-H23 | |
| astrocytoma SW1783 | 15.8 | Lung ca. (non-s. cell) | 18.2 |
| | | HOP-62 | |
| neuro*; met SK-N-AS | 10.2 | Lung ca. (non-s. cl) | 10.7 |
| | | NCI-H522 | |
| astrocytoma SF-539 | 9.5 | Lung ca. (squam.) SW | 8.0 |
| | | 900 | |
| astrocytoma SNB-75 | 0.0 | Lung ca. (squam.) | 9.3 |
| | | NCI-H596 | |
| glioma SNB-19 | 16.6 | Mammary gland | 13.0 |
| glioma U251 | 5.2 | Breast ca.* (pl. ef) | 3.1 |
| | | MCF-7 | |
| glioma SF-295 | 0.0 | Breast ca.* (pl. ef) | 8.3 |
| | | MDA-MB-231 | |
| Heart (Fetal) | 24.1 | Breast ca.* (pl. ef) | 4.0 |
| | | T47D | |
| Heart | 33.0 | Breast ca. BT-549 | 6.1 |
| Skeletal muscle (Fetal) | 6.5 | Breast ca. MDA-N | 0.0 |
| Skeletal muscle | 10.8 | Ovary | 7.2 |
| Bone marrow | 5.7 | Ovarian ca. OVCAR-3 | 16.7 |
| Thymus | 0.0 | Ovarian ca. OVCAR-4 | 13.2 |
| Spleen | 33.0 | Ovarian ca. OVCAR-5 | 10.6 |
| Lymph node | 13.7 | Ovarian ca. OVCAR-8 | 0.0 |
| Colorectal | 28.5 | Ovarian ca. IGROV-1 | 9.3 |
| Stomach | 4.8 | Ovarian ca. (ascites) | 0.0 |
| | | SK-OV-3 | |
| Small intestine | 8.6 | Uterus | 0.0 |
| Colon ca. SW480 | 0.0 | Placenta | 8.0 |
| Colon ca.* SW620 | 4.4 | Prostate | 0.0 |
| (SW480 met) | | | |
| Colon ca. HT29 | 5.3 | Prostate ca.* (bone | 0.0 |
| | | met) PC-3 | |
| Colon ca. HCT-116 | 19.9 | Testis | 0.0 |
| Colon ca. CaCo-2 | 9.7 | Melanoma Hs688(A).T | 0.0 |
| CC Well to Mod Diff | 0.0 | Melanoma* (met) | 0.0 |
| (ODO3866) | | Hs688(B).T | |
| Colon ca. HCC-2998 | 0.0 | Melanoma UACC-62 | 6.6 |
| Gastric ca. (liver met) | 0.0 | Melanoma M14 | 5.1 |
| NCI-N87 | | | |
| Bladder | 100.0 | Melanoma LOX IMVI | 8.2 |
| Trachea | 4.4 | Melanoma* (met) SK- | 8.7 |
| | | MEL-5 | |
| Kidney | 25.7 | Adipose | 79.6 |

TABLE 43
Panel 2D

| | Rel. Exp. (%) | | Rel. Exp. (%) |
|---|---|---|---|
| | Ag2423, | | Ag2423, |
| Tissue Name | Run 159338041 | Tissue Name | Run 159338041 |
| Normal Colon | 34.6 | Kidney Margin | 0.0 |
| | | 8120608 | |
| CC Well to Mod Diff | 34.2 | Kidney Cancer 8120613 | 0.0 |
| (ODO3866) | | | |
| CC Margin (ODO3866) | 38.7 | Kidney Margin | 0.0 |
| | | 8120614 | |
| CC Gr.2 rectosigmoid | 9.1 | Kidney Cancer 9010320 | 0.0 |
| (ODO3868) | | | |
| CC Margin (ODO3868) | 11.0 | Kidney Margin | 0.0 |
| | | 9010321 | |
| CC Mod Diff (ODO3920) | 12.9 | Normal Uterus | 8.9 |
| CC Margin (ODO3920) | 17.7 | Uterine Cancer 064011 | 12.2 |
| CC Gr.2 ascend colon | 79.0 | Normal Thyroid | 2.6 |
| (ODO3921) | | | |
| CC Margin (ODO3921) | 17.1 | Thyroid Cancer | 5.6 |
| CC from Partial | 24.1 | Thyroid Cancer | 7.7 |
| Hepatectomy (ODO4309) | | A302152 | |
| Mets | | | |
| Liver Margin (ODO4309) | 17.8 | Thyroid Margin | 10.9 |
| | | A302153 | |
| Colon mets to lung | 2.0 | Normal Breast | 7.2 |
| (OD04451-01) | | | |
| Lung Margin (OD04451-02) | 8.0 | Breast Cancer | 2.4 |
| Normal Prostate 6546-1 | 2.8 | Breast Cancer | 16.0 |
| | | (OD04590-01) | |
| Prostate Cancer (OD04410) | 45.4 | Breast Cancer Mets | 19.5 |
| | | (OD04590-03) | |
| Prostate Margin (OD04410) | 27.4 | Breast Cancer | 11.2 |
| | | Metastasis | |
| Prostate Cancer (OD04720- | 9.8 | Breast Cancer | 10.9 |
| 01) | | | |
| Prostate Margin (OD04720- | 29.3 | Breast Cancer | 3.6 |
| 02) | | | |
| Normal Lung | 38.2 | Breast Cancer 9100266 | 12.9 |
| Lung Met to Muscle | 36.3 | Breast Margin 9100265 | 4.6 |
| (ODO4286) | | | |
| Muscle Margin (ODO4286) | 9.9 | Breast Cancer A209073 | 15.4 |
| Lung Malignant Cancer | 15.7 | Breast Margin | 6.1 |
| (OD03126) | | A2090734 | |
| Lung Margin (OD03126) | 12.0 | Normal Liver | 3.8 |
| Lung Cancer (OD04404) | 14.4 | Liver Cancer | 9.1 |
| Lung Margin (OD04404) | 10.1 | Liver Cancer 1025 | 3.8 |
| Lung Cancer (OD04565) | 7.4 | Liver Cancer 1026 | 2.7 |
| Lung Margin (OD04565) | 0.0 | Liver Cancer 6004-T | 3.2 |
| Lung Cancer (OD04237-01) | 43.8 | Liver Tissue 6004-N | 3.4 |
| Lung Margin (OD04237-02) | 12.9 | Liver Cancer 6005-T | 3.4 |
| Ocular Mel Met to Liver | 3.0 | Liver Tissue 6005-N | 1.6 |
| (ODO4310) | | | |
| Liver Margin (ODO4310) | 4.1 | Normal Bladder | 36.9 |
| Melanoma Metastasis | 33.2 | Bladder Cancer | 10.0 |
| Lung Margin (OD04321) | 23.7 | Bladder Cancer | 22.4 |
| Normal Kidney | 12.4 | Bladder Cancer | 100.0 |
| | | (OD04718-01) | |
| Kidney Ca, Nuclear grade 2 | 6.8 | Bladder Normal | 13.1 |
| (OD04338) | | Adjacent (OD04718-03) | |
| Kidney Margin (OD04338) | 6.2 | Normal Ovary | 3.0 |
| Kidney Ca Nuclear grade | 20.7 | Ovarian Cancer | 18.2 |
| 1/2 (OD04339) | | | |
| Kidney Margin (OD04339) | 11.8 | Ovarian Cancer | 47.6 |
| | | (OD04768-07) | |
| Kidney Ca, Clear cell type | 29.9 | Ovary Margin | 6.1 |
| (OD04340) | | (OD04768-08) | |
| Kidney Margin (OD04340) | 11.0 | Normal Stomach | 6.2 |
| Kidney Ca, Nuclear grade 3 | 5.8 | Gastric Cancer 9060358 | 0.0 |
| (OD04348) | | | |
| Kidney Margin (OD04348) | 9.8 | Stomach Margin | 42.3 |
| | | 9060359 | |
| Kidney Cancer (OD04622- | 0.0 | Gastric Cancer 9060395 | 37.4 |
| 01) | | | |
| Kidney Margin (OD04622- | 0.0 | Stomach Margin | 47.0 |
| 03) | | 9060394 | |
| Kidney Cancer (OD04450- | 7.5 | Gastric Cancer 9060397 | 76.3 |
| 01) | | | |
| Kidney Margin (OD04450- | 5.4 | Stomach Margin | 3.1 |
| 03) | | 9060396 | |
| Kidney Cancer 8120607 | 0.0 | Gastric Cancer 064005 | 35.6 |

TABLE 44
Panel 4D

| | Rel. Exp. (%) | | Rel. Exp. (%) |
|---|---|---|---|
| | Ag2423, Run | | Ag2423, Run |
| Tissue Name | 159338325 | Tissue Name | 159338325 |
| Secondary Th1 act | 2.1 | HUVEC IL-1beta | 1.9 |
| Secondary Th2 act | 4.8 | HUVEC IFN gamma | 0.0 |
| Secondary Tr1 act | 1.4 | HUVEC TNF alpha + IFN | 0.0 |
| | | gamma | |
| Secondary Th1 rest | 7.5 | HUVEC TNF alpha + IL4 | 0.0 |
| Secondary Th2 rest | 10.2 | HUVEC IL-11 | 0.0 |
| Secondary Tr1 rest | 2.0 | Lung Microvascular EC none | 4.7 |
| Primary Th1 act | 2.3 | Lung Microvascular EC | 3.5 |
| | | TNF alpha + IL-1beta | |
| Primary Th2 act | 100.0 | Microvascular Dermal EC | 8.0 |
| | | none | |
| Primary Tr1 act | 3.4 | Microsvasular Dermal EC | 15.3 |
| | | TNF alpha + IL-1beta | |
| Primary Th1 rest | 0.9 | Bronchial epithelium | 1.3 |
| | | TNF alpha + IL1beta | |
| Primary Th2 rest | 3.8 | Small airway epithelium | 3.6 |
| | | none | |
| Primary Tr1 rest | 1.4 | Small airway epithelium | 0.0 |
| | | TNF alpha + IL-1beta | |
| CD45RA CD4 | 1.6 | Coronery artery SMC rest | 0.0 |
| lymphocyte act | | | |
| CD45RO CD4 | 0.0 | Coronery artery SMC | 0.0 |
| lymphocyte act | | TNF alpha + IL-1beta | |
| CD8 lymphocyte act | 5.2 | Astrocytes rest | 5.1 |
| Secondary CD8 | 0.0 | Astrocytes TNF alpha + IL- | 0.0 |
| lymphocyte rest | | 1beta | |
| Secondary CD8 | 1.1 | KU-812 (Basophil) rest | 0.0 |
| lymphocyte act | | | |
| CD4 lymphocyte none | 2.2 | KU-812 (Basophil) | 2.1 |
| | | PMA/ionomycin | |
| 2ry Th1/Th2/Tr1_anti- | 1.5 | CCD1106 (Keratinocytes) | 1.5 |
| CD95 CH11 | | none | |
| LAK cells rest | 7.6 | CCD1106 (Keratinocytes) | 2.0 |
| | | TNF alpha + IL-1beta | |
| LAK cells IL-2 | 5.8 | Liver cirrhosis | 2.1 |
| LAK cells IL-2 + IL-12 | 1.4 | Lupus kidney | 4.8 |
| LAK cells IL-2 + IFN | 0.0 | NCI-H292 none | 1.6 |
| gamma | | | |
| LAK cells IL-2 + IL-18 | 1.7 | NCI-H292 IL-4 | 6.6 |
| LAK cells | 1.7 | NCI-H292 IL-9 | 0.0 |
| PMA/ionomycin | | | |
| NK Cells IL-2 rest | 0.9 | NCI-H292 IL-13 | 0.0 |
| Two Way MLR 3 day | 0.0 | NCI-H292 IFN gamma | 1.3 |
| Two Way MLR 5 day | 1.8 | HPAEC none | 0.0 |
| Two Way MLR 7 day | 8.5 | HPAEC TNF alpha + IL-1beta | 1.4 |
| PBMC rest | 1.8 | Lung fibroblast none | 0.9 |
| PBMC PWM | 1.8 | Lung fibroblast TNF alpha + | 6.0 |
| | | IL-1beta | |
| PBMC PHA-L | 3.7 | Lung fibroblast IL-4 | 6.3 |
| Ramos (B cell) none | 2.0 | Lung fibroblast IL-9 | 1.0 |
| Ramos (B cell) | 0.0 | Lung fibroblast IL-13 | 0.9 |
| ionomycin | | | |
| B lymphocytes PWM | 0.0 | Lung fibroblast IFN gamma | 0.8 |
| B lymphocytes CD40L | 0.0 | Dermal fibroblast CCD1070 | 2.2 |
| and IL-4 | | rest | |
| EOL-1 dbcAMP | 4.2 | Dermal fibroblast CCD1070 | 2.0 |
| | | TNF alpha | |
| EOL-1 dbcAMP | 0.0 | Dermal fibroblast CCD1070 | 0.0 |
| PMA/ionomycin | 0.0 | IL-1beta | |
| Dendritic cells none | 2.3 | Dermal fibroblast IFN | 0.0 |
| | | gamma | |
| Dendritic cells LPS | 4.3 | Dermal fibroblast IL-4 | 0.0 |
| Dendritic cells anti- | 0.0 | IBD Colitis 2 | 3.1 |
| CD40 | | | |
| Monocytes rest | 1.7 | IBD Crohn's | 1.8 |
| Monocytes LPS | 28.3 | Colon | 0.0 |
| Macrophages rest | 20.7 | Lung | 0.0 |
| Macrophages LPS | 1.1 | Thymus | 2.0 |
| HUVEC none | 2.0 | Kidney | 3.7 |
| HUVEC starved | 1.5 | | |

CNS_neurodegeneration_v1.0 Summary: Ag2423 Expression is low/undetected in all samples in this panel (CT>35). (Data not shown.)

Panel 1.3D Summary: Ag2423 This gene is expressed exclusively in a sample derived from bladder tissue. Thus, the expression of this gene could be used to distinguish bladder tissue from other tissues in the panel.

Panel 2D Summary: Ag2423 The expression of this gene is highest and almost exclusive to a sample derived from bladder cancer. This result is consistent with the expression detected in Panel 1.3D. Thus, the expression of this gene could be used to distinguish bladder cancer tissue from other tissues in the panel. Moreover, the therapeutic modulation of this gene, through the use of small molecule drugs, antibodies or protein therapeutics might be of benefit in the treatment of bladder cancer.

Panel 4D Summary: Ag2423 The expression of this gene is highest and almost exclusive to primary activated Th2 cells (CT 32.6). Very low expression of this transcript is found in activated LPS and macrophages (CT 34.9). This transcript encodes for a 26s proteasome like protein which is an essential component of the cellular protein degradation machinery. Some studies (reference 1) indicate a potential role for proteasomes in the regulation of signal transduction in T and B lymphocytes. This novel 26S proteasome may be involved in a more specific Th2 signalling pathway. Therefore, this gene product may be useful as a potential therapeutic target for attenuation of hyperactive Th2 response such as observed in allergic diseases (rhinitis, atopic skin diseases, asthma).

Reference:

Biochim Biophys Acta Jan. 6; 1999 1453(1):92-104 Proteasome participates in the alteration of signal transduction in T and B lymphocytes following trauma-hemorrhage. Samy T S, Schwacha M G, Chung C S, Cioffi W G, Bland K I, Chaudry I H.

Department of Surgery, Brown University School of Medicine, Providence, R.I., USA.

Proteasomes are essential components of the cellular protein degradation machinery. They are nonlysosomal and their participation is critical for (1) the removal of short lived proteins involved in metabolic regulation and cell proliferation, (2) the control of the activities of regulators involved in gene transcription, such as nuclear factor-kappa B (NF-kappa B) and signal transducer and activator of transcription (STAT1), and (3) processing of antigenic peptides for MHC class I presentation. Trauma-hemorrhage induces profound immunosuppression which is characterized by reduced splenocyte proliferation, interleukin (IL)-2 and interferon (IFN)-gamma productive capacity, increased activation of transcription factors NF-kappa B and STAT1in splenic T lymphocytes, reduced macrophage antigen presentation capacity and inordinate release of proinflammatory cytokines, such as IL-6 and tumor necrosis factor-alpha. Furthermore, it appears that the activity of several regulatory proteins involved in immune function is altered by trauma-hemorrhage. Since proteasomes are involved in regulation and removal of regulatory proteins, we hypothesized that trauma-hemorrhage alters proteasomal activity in splenic lymphocytes. The data showed that activities of 26s proteasome from CD3+CD4+ and CD3+CD8+ splenic T lymphocytes were enhanced following trauma-hemorrhage which was associated with increased expression of NF-kappa B and STAT1. On the other hand, trauma-hemorrhage attenuated the activity of 26s proteasome from splenic B lymphocytes which was restored upon IFN-gamma stimulation and correlated with increased expression of NF-kappa B. These studies indicate a potential role for proteasomes in the regulation of signal transduction in splenic T and B lymphocytes following trauma-hemorrhage, and also suggest them as potential therapeutic targets for attenuation of immune suppression associated with this form of injury.

NOV6

Expression of gene NOV6 was assessed using the primer-probe sets Ag1508, Ag1586, Ag2011 and Ag2284, described in Tables 45, 46, 47 and 48. Results of the RTQ-PCR runs are shown in Tables 49, 50, 51, 52, 53 and 54.

TABLE 45

Probe Name Ag1508

| | | | Start | SEQ ID |
|---|---|---|---|---|
| Primers | Sequences | Length | Position | NO: |
| Forward | 5′-atttggctatcccttcaggtt-3′ | 21 | 238 | 160 |
| Probe | TET-5′-cggatccaatatgagatgcccctct-3′-TAMRA | 25 | 263 | 161 |
| Reverse | 5′-gtcttggagctggactcttcat-3′ | 22 | 291 | 162 |

TABLE 46

Probe Name Ag1586

| | | | Start | SEQ ID |
|---|---|---|---|---|
| Primers | Sequences | Length | Position | NO: |
| Forward | 5′-accaggatgagtttgtgtcatc-3′ | 22 | 1583 | 163 |
| Probe | TET-5′-ctcaagatcccttcggacacgctgt-3′-TAMRA | 25 | 1609 | 164 |
| Reverse | 5′-tgcggaagctgtacacatagta-3′ | 22 | 1657 | 165 |

TABLE 47

uz,17/28 Probe Name Ag2011

| | | | Start | SEQ ID |
|---|---|---|---|---|
| Primers | Sequences | Length | Position | NO: |
| Forward | 5′-accaggatgagtttgtgtcatc-3′ | 22 | 1583 | 166 |
| Probe | TET-5′-ctcaagatcccttcggacacgctgt-3′-TAMRA | 25 | 1609 | 167 |
| Reverse | 5′-tgcggaagctgtacacatagta-3′ | 22 | 1657 | 168 |

TABLE 48

Probe Name Ag2284

| | | | | |
|---|---|---|---|---|
| | | | Start | SEQ ID |
| Primers | Sequences | Length | Position | NO: |

| | | | | |
|---|---|---|---|---|
| Forward | 5′-tagttatctacctgcgcttcca-3′ | 22 | 399 | 169 |
| Probe | TET-5′-cctacacagagaacaaacgcttcccg-3′-TAMRA | 26 | 426 | 170 |
| Reverse | 5′-gaaggtgaaggagacagtcaca-3′ | 22 | 466 | 171 |

TABLE 49
Panel 1.2

| | Rel. Exp. (%) | | Rel. Exp. (%) |
|---|---|---|---|
| | Ag1508, | | Ag1508, |
| Tissue Name | Run 141937122 | Tissue Name | Run 141937122 |
| Endothelial cells | 0.0 | Renal ca. 786-0 | 0.0 |
| Heart (Fetal) | 0.9 | Renal ca. A498 | 0.0 |
| Pancreas | 0.1 | Renal ca. RXF 393 | 0.0 |
| Pancreatic ca. CAPAN 2 | 0.0 | Renal ca. ACHN | 0.0 |
| Adrenal Gland | 2.7 | Renal ca. UO-31 | 0.0 |
| Thyroid | 0.1 | Renal ca. TK-10 | 0.0 |
| Salivary gland | 0.9 | Liver | 0.3 |
| Pituitary gland | 0.0 | Liver (fetal) | 0.1 |
| Brain (fetal) | 0.0 | Liver ca. (hepatoblast) | 0.0 |
| | | HepG2 | |
| Brain (whole) | 0.0 | Lung | 0.0 |
| Brain (amygdala) | 0.0 | Lung (fetal) | 0.0 |
| Brain (cerebellum) | 0.1 | Lung ca. (small cell) | 0.0 |
| | | LX-1 | |
| Brain (hippocampus) | 0.1 | Lung ca. (small cell) | 0.0 |
| | | NCI-H69 | |
| Brain (thalamus) | 0.0 | Lung ca. (s. cell var.) | 0.0 |
| | | SHP-77 | |
| Cerebral Cortex | 0.3 | Lung ca. (large | 0.0 |
| | | cell)NCI-H460 | |
| Spinal cord | 0.0 | Lung ca. (non-sm. cell) | 0.0 |
| | | A549 | |
| glio/astro U87-MG | 0.0 | Lung ca. (non-s. cell) | 0.0 |
| | | NCI-H23 | |
| glio/astro U-118-MG | 0.1 | Lung ca. (non-s. cell) | 0.0 |
| | | HOP-62 | |
| astrocytoma SW1783 | 0.0 | Lung ca. (non-s. cl) | 9.4 |
| | | NCI-H522 | |
| neuro*; met SK-N-AS | 0.0 | Lung ca. (squam.) SW | 0.2 |
| | | 900 | |
| astrocytoma SF-539 | 0.0 | Lung ca. (squam.) | 0.0 |
| | | NCI-H596 | |
| astrocytoma SNB-75 | 0.0 | Mammary gland | 0.0 |
| glioma SNB-19 | 0.0 | Breast ca.* (pl. ef) | 0.0 |
| | | MCF-7 | |
| glioma U251 | 0.0 | Breast ca.* (pl. ef) | 0.0 |
| | | MDA-MB-231 | |
| glioma SF-295 | 0.0 | Breast ca.* (pl. ef) | 0.0 |
| | | T47D | |
| Heart | 10.7 | Breast ca. BT-549 | 0.0 |
| Skeletal Muscle | 100.0 | Breast ca. MDA-N | 0.0 |
| Bone marrow | 0.1 | Ovary | 0.5 |
| Thymus | 0.0 | Ovarian ca. OVCAR-3 | 0.0 |
| Spleen | 0.0 | Ovarian ca. OVCAR-4 | 0.0 |
| Lymph node | 0.0 | Ovarian ca. OVCAR-5 | 0.0 |
| Colorectal | 0.0 | Ovarian ca. OVCAR-8 | 0.0 |
| Stomach | 0.1 | Ovarian ca. IGROV-1 | 0.0 |
| Small intestine | 0.2 | Ovarian ca. (ascites) | 0.0 |
| | | SK-OV-3 | |
| Colon ca. SW480 | 0.0 | Uterus | 0.2 |
| Colon ca.* SW620 | 0.0 | Placenta | 0.0 |
| (SW480 met) | | | |
| Colon ca. HT29 | 0.0 | Prostate | 0.4 |
| Colon ca. HCT-116 | 0.1 | Prostate ca.* (bone | 0.0 |
| | | met) PC-3 | |
| Colon ca. CaCo-2 | 0.0 | Testis | 0.2 |
| CC Well to Mod Diff | 0.0 | Melanoma Hs688(A).T | 0.0 |
| (ODO3866) | | | |
| Colon ca. HCC-2998 | 0.0 | Melanoma* (met) | 0.0 |
| | | Hs688(B).T | |
| Gastric ca. (liver met) | 0.0 | Melanoma UACC-62 | 0.1 |
| NCI-N87 | | | |
| Bladder | 0.2 | Melanoma M14 | 0.0 |
| Trachea | 0.0 | Melanoma LOX IMVI | 0.0 |
| Kidney | 8.9 | Melanoma* (met) SK- | 0.0 |
| | | MEL-5 | |
| Kidney (fetal) | 0.6 | | |

TABLE 50
Panel 1.3D

| | Rel. | Rel. | Rel. | | Rel. | Rel. | Rel. |
|---|---|---|---|---|---|---|---|
| | Exp. (%) | Exp. (%) | Exp. (%) | | Exp. (%) | Exp. (%) | Exp. (%) |
| | Ag1586, | Ag2011, | Ag2284, | | Ag1586, | Ag2011, | Ag2284, |
| | Run | Run | Run | Tissue | Run | Run | Run |
| Tissue Name | 146473155 | 147816085 | 167985231 | Name | 146473155 | 147816085 | 167985231 |
| Liver | 29.9 | 37.6 | 0.2 | Kidney | 3.8 | 3.7 | 1.6 |
| adenocarcinoma | | | | (fetal) | | | |
| Pancreas | 1.7 | 0.7 | 0.3 | Renal ca. | 6.1 | 11.7 | 0.0 |
| | | | | 786-0 | | | |
| Pancreatic ca. | 6.3 | 9.6 | 0.0 | Renal ca. | 25.0 | 25.9 | 0.0 |
| CAPAN 2 | | | | A498 | | | |
| Adrenal gland | 2.6 | 2.5 | 0.5 | Renal ca. | 4.5 | 5.0 | 0.0 |
| | | | | RXF 393 | | | |
| Thyroid | 2.5 | 1.8 | 1.2 | Renal ca. | 8.8 | 11.3 | 0.0 |
| | | | | ACHN | | | |
| Salivary gland | 1.9 | 2.2 | 0.4 | Renal ca. | 15.0 | 15.0 | 0.0 |
| | | | | UO-31 | | | |
| Pituitary gland | 0.9 | 1.5 | 0.1 | Renal ca. | 4.4 | 4.6 | 0.0 |
| | | | | TK-10 | | | |
| Brain (fetal) | 12.2 | 13.1 | 0.0 | Liver | 0.2 | 0.1 | 0.4 |
| Brain (whole) | 9.7 | 10.7 | 0.2 | Liver (fetal) | 0.7 | 0.8 | 0.1 |
| Brain | 9.5 | 9.9 | 0.2 | Liver ca. | 16.8 | 12.8 | 0.1 |
| (amygdala) | | | | (hepatoblast) | | | |
| | | | | HepG2 | | | |
| Brain | 3.3 | 2.3 | 0.1 | Lung | 5.0 | 5.1 | 0.0 |
| (cerebellum) | | | | | | | |
| Brain | 24.7 | 21.0 | 0.1 | Lung (fetal) | 7.4 | 8.1 | 0.1 |
| (hippocampus) | | | | | | | |
| Brain | 0.9 | 1.3 | 0.1 | Lung ca. | 16.8 | 12.1 | 0.0 |
| (substantia | | | | (small cell) | | | |
| nigra) | | | | LX-1 | | | |
| Brain | 4.7 | 3.7 | 0.1 | Lung ca. | 18.4 | 23.7 | 0.0 |
| (thalamus) | | | | (small cell) | | | |
| | | | | NCI-H69 | | | |
| Cerebral Cortex | 75.8 | 71.2 | 0.2 | Lung ca. | 8.5 | 7.2 | 0.0 |
| | | | | (s.cell var.) | | | |
| | | | | SHP-77 | | | |
| Spinal cord | 2.0 | 2.4 | 0.1 | Lung ca. | 10.7 | 10.1 | 0.0 |
| | | | | (large | | | |
| | | | | cell)NCI- | | | |
| | | | | H460 | | | |
| glio/astro U87- | 15.3 | 17.9 | 0.0 | Lung ca. | 3.2 | 4.1 | 0.0 |
| MG | | | | (non-sm. | | | |
| | | | | cell) A549 | | | |
| glio/astro U- | 38.2 | 41.2 | 0.2 | Lung ca. | 23.2 | 24.7 | 0.5 |
| 118-MG | | | | (non-s.cell) | | | |
| | | | | NCI-H23 | | | |
| astrocytoma | 8.3 | 10.4 | 0.1 | Lung ca. | 18.9 | 15.7 | 0.0 |
| SW1783 | | | | (non-s.cell) | | | |
| | | | | HOP-62 | | | |
| neuro*; met | 23.5 | 24.3 | 0.0 | Lung ca. | 5.6 | 7.5 | 8.1 |
| SK-N-AS | | | | (non-s.cl) | | | |
| | | | | NCI-H522 | | | |
| astrocytoma SF- | 19.6 | 38.4 | 0.0 | Lung ca. | 13.0 | 13.1 | 0.2 |
| 539 | | | | (squam.) SW | | | |
| | | | | 900 | | | |
| astrocytoma | 44.4 | 45.1 | 0.1 | Lung ca. | 6.5 | 5.7 | 0.0 |
| SNB-75 | | | | (squam.) | | | |
| | | | | NCI-H596 | | | |
| glioma SNB-19 | 26.2 | 12.2 | 0.0 | Mammary | 11.5 | 9.3 | 0.2 |
| | | | | gland | | | |
| glioma U251 | 16.4 | 16.2 | 0.1 | Breast ca.* | 14.1 | 14.4 | 0.0 |
| | | | | (pl.ef) | | | |
| | | | | MCF-7 | | | |
| glioma SF-295 | 26.4 | 36.9 | 0.0 | Breast ca.* | 82.9 | 87.1 | 0.0 |
| | | | | (pl.ef) | | | |
| | | | | MDA-MB- | | | |
| | | | | 231 | | | |
| Heart (Fetal) | 80.7 | 95.3 | 1.8 | Breast ca.* | 6.1 | 4.6 | 0.1 |
| | | | | (pl. ef) | | | |
| | | | | T47D | | | |
| Heart | 2.8 | 1.9 | 2.3 | Breast ca. | 13.6 | 11.2 | 0.2 |
| | | | | BT-549 | | | |
| Skeletal muscle | 85.3 | 87.7 | 100.0 | Breast ca. | 28.1 | 31.6 | 0.0 |
| (Fetal) | | | | MDA-N | | | |
| Skeletal muscle | 2.1 | 2.4 | 88.3 | Ovary | 20.9 | 19.5 | 0.8 |
| Bone marrow | 0.6 | 0.3 | 0.2 | Ovarian ca. | 33.0 | 40.1 | 0.0 |
| | | | | OVCAR-3 | | | |
| Thymus | 2.6 | 2.3 | 0.0 | Ovarian ca. | 5.5 | 5.4 | 0.0 |
| | | | | OVCAR-4 | | | |
| Spleen | 2.9 | 2.6 | 0.0 | Ovarian ca. | 10.9 | 13.1 | 0.1 |
| | | | | OVCAR-5 | | | |
| Lymph node | 5.1 | 5.2 | 0.1 | Ovarian ca. | 17.4 | 18.3 | 0.1 |
| | | | | OVCAR-8 | | | |
| Colorectal | 5.2 | 3.9 | 0.0 | Ovarian ca. | 4.5 | 5.3 | 0.0 |
| | | | | IGROV-1 | | | |
| Stomach | 3.7 | 5.6 | 0.2 | Ovarian ca. | 25.7 | 22.4 | 0.1 |
| | | | | (ascites) SK- | | | |
| | | | | OV-3 | | | |
| Small intestine | 1.6 | 1.3 | 0.2 | Uterus | 2.7 | 2.4 | 1.0 |
| Colon ca. | 45.4 | 55.5 | 0.1 | Placenta | 6.7 | 10.2 | 0.2 |
| SW480 | | | | | | | |
| Colon ca.* | 11.3 | 11.1 | 0.0 | Prostate | 0.4 | 1.4 | 0.2 |
| SW620 (SW480 | | | | | | | |
| met) | | | | | | | |
| Colon ca. HT29 | 13.3 | 13.3 | 0.0 | Prostate ca.* | 8.4 | 11.3 | 0.0 |
| | | | | (bone met) | | | |
| | | | | PC-3 | | | |
| Colon ca. HCT- | 10.5 | 10.5 | 0.2 | Testis | 8.1 | 8.5 | 1.1 |
| 116 | | | | | | | |
| Colon ca. | 24.0 | 23.0 | 0.1 | Melanoma | 59.0 | 86.5 | 0.0 |
| CaCo-2 | | | | Hs688(A).T | | | |
| CC Well to | 19.1 | 16.6 | 0.1 | Melanoma* | 100.0 | 100.0 | 0.0 |
| Mod Diff | | | | (met) | | | |
| (ODO3866) | | | | Hs688(B).T | | | |
| Colon ca. HCC- | 25.7 | 20.3 | 0.0 | Melanoma | 17.6 | 19.5 | 0.1 |
| 2998 | | | | UACC-62 | | | |
| Gastric ca. | 59.9 | 62.9 | 0.1 | Melanoma | 16.3 | 21.9 | 0.0 |
| (liver met) NCI- | | | | M14 | | | |
| N87 | | | | | | | |
| Bladder | 1.8 | 4.6 | 0.2 | Melanoma | 3.6 | 5.8 | 0.0 |
| | | | | LOX IMVI | | | |
| Trachea | 6.9 | 5.6 | 0.1 | Melanoma* | 12.9 | 22.1 | 0.0 |
| | | | | (met) SK- | | | |
| | | | | MEL-5 | | | |
| Kidney | 0.8 | 0.7 | 2.8 | Adipose | 5.6 | 4.5 | 0.7 |

TABLE 51
Panel 2.2

| | Rel. Exp. (%) | | Rel. Exp. (%) |
|---|---|---|---|
| | Ag2011, | | Ag2011, |
| Tissue Name | Run 174154748 | Tissue Name | Run 174154748 |
| Normal Colon | 24.7 | Kidney Margin | 68.3 |
| | | (OD04348) | |
| Colon cancer (OD06064) | 48.6 | Kidney malignant cancer | 25.0 |
| | | (OD06204B) | |
| Colon Margin (OD06064) | 4.9 | Kidney normal adjacent | 7.4 |
| | | tissue (OD06204E) | |
| Colon cancer (OD06159) | 9.3 | Kidney Cancer | 34.4 |
| | | (OD04450-01) | |
| Colon Margin (OD06159) | 19.5 | Kidney Margin | 18.4 |
| | | (OD04450-03) | |
| Colon cancer (OD06297- | 11.7 | Kidney Cancer 8120613 | 9.7 |
| 04) | | | |
| Colon Margin (OD06297- | 12.5 | Kidney Margin 8120614 | 18.8 |
| 015) | | | |
| CC Gr.2 ascend colon | 17.3 | Kidney Cancer 9010320 | 16.2 |
| (ODO3921) | | | |
| CC Margin (ODO3921) | 14.2 | Kidney Margin 9010321 | 13.8 |
| Colon cancer metastasis | 8.6 | Kidney Cancer 8120607 | 37.1 |
| (OD06104) | | | |
| Lung Margin (OD06104) | 8.3 | Kidney Margin 8120608 | 7.0 |
| Colon mets to lung | 23.0 | Normal Uterus | 21.9 |
| (OD04451-01) | | | |
| Lung Margin (OD04451- | 32.8 | Uterine Cancer 064011 | 13.7 |
| 02) | | | |
| Normal Prostate | 4.8 | Normal Thyroid | 2.4 |
| Prostate Cancer | 4.9 | Thyroid Cancer | 8.1 |
| (OD04410) | | | |
| Prostate Margin | 8.8 | Thyroid Cancer A302152 | 35.4 |
| (OD04410) | | | |
| Normal Ovary | 32.3 | Thyroid Margin A302153 | 8.7 |
| Ovarian cancer | 32.1 | Normal Breast | 29.7 |
| (OD06283-03) | | | |
| Ovarian Margin | 13.8 | Breast Cancer | 11.9 |
| (OD06283-07) | | | |
| Ovarian Cancer | 19.9 | Breast Cancer | 47.6 |
| Ovarian cancer | 9.2 | Breast Cancer (OD04590- | 25.5 |
| (OD06145) | | 01) | |
| Ovarian Margin | 8.6 | Breast Cancer Mets | 38.4 |
| (OD06145) | | (OD04590-03) | |
| Ovarian cancer | 13.0 | Breast Cancer Metastasis | 30.1 |
| (OD06455-03) | | | |
| Ovarian Margin | 2.1 | Breast Cancer | 41.5 |
| (OD06455-07) | | | |
| Normal Lung | 27.2 | Breast Cancer 9100266 | 9.2 |
| Invasive poor diff. lung | 28.5 | Breast Margin 9100265 | 18.2 |
| adeno (ODO4945-01 | | | |
| Lung Margin (ODO4945- | 15.0 | Breast Cancer A209073 | 14.9 |
| 03) | | | |
| Lung Malignant Cancer | 30.4 | Breast Margin A2090734 | 37.6 |
| (OD03126) | | | |
| Lung Margin (OD03126) | 15.9 | Breast cancer (OD06083) | 55.9 |
| Lung Cancer (OD05014A) | 39.5 | Breast cancer node | 48.6 |
| | | metastasis (OD06083) | |
| Lung Margin (OD05014B) | 22.1 | Normal Liver | 10.4 |
| Lung cancer (OD06081) | 23.7 | Liver Cancer 1026 | 9.1 |
| Lung Margin (OD06081) | 16.8 | Liver Cancer 1025 | 20.7 |
| Lung Cancer (OD04237- | 9.0 | Liver Cancer 6004-T | 12.2 |
| 01) | | | |
| Lung Margin (OD04237- | 41.5 | Liver Tissue 6004-N | 8.0 |
| 02) | | | |
| Ocular Mel Met to Liver | 100.0 | Liver Cancer 6005-T | 36.6 |
| (ODO4310) | | | |
| Liver Margin (ODO4310) | 4.2 | Liver Tissue 6005-N | 25.0 |
| Melanoma Metastasis | 47.0 | Liver Cancer | 4.5 |
| Lung Margin (OD04321) | 28.1 | Normal Bladder | 18.7 |
| Normal Kidney | 12.3 | Bladder Cancer | 17.2 |
| Kidney Ca, Nuclear grade | 18.3 | Bladder Cancer | 72.7 |
| 2 (OD04338) | | | |
| Kidney Margin | 18.0 | Normal Stomach | 33.4 |
| (OD04338) | | | |
| Kidney Ca Nuclear grade | 83.5 | Gastric Cancer 9060397 | 9.6 |
| 1/2 (OD04339) | | | |
| Kidney Margin | 10.4 | Stomach Margin 9060396 | 10.4 |
| (OD04339) | | | |
| Kidney Ca, Clear cell type | 22.2 | Gastric Cancer 9060395 | 7.6 |
| (OD04340) | | | |
| Kidney Margin | 12.7 | Stomach Margin 9060394 | 19.6 |
| (OD04340) | | | |
| Kidney Ca, Nuclear grade | 15.7 | Gastric Cancer 064005 | 17.4 |
| 3 (OD04348) | | | |

TABLE 52
Panel 2D

| | Rel. Exp. (%) | Rel. Exp. (%) | | Rel. Exp. (%) | Rel. Exp. (%) |
|---|---|---|---|---|---|
| | Ag1508, Run | Ag1586, Run | | Ag1508, Run | Ag1586, Run |
| Tissue Name | 144982575 | 162624476 | Tissue Name | 144982575 | 162624476 |
| Normal Colon | 2.2 | 34.9 | Kidney Margin | 11.3 | 14.2 |
| | | | 8120608 | | |
| CC Well to Mod | 0.1 | 28.3 | Kidney Cancer | 3.6 | 30.4 |
| Diff (ODO3866) | | | 8120613 | | |
| CC Margin | 1.4 | 9.2 | Kidney Margin | 11.0 | 17.7 |
| (ODO3866) | | | 8120614 | | |
| CC Gr.2 | 0.1 | 25.9 | Kidney Cancer | 0.7 | 57.0 |
| rectosigmoid | | | 9010320 | | |
| (ODO3868) | | | | | |
| CC Margin | 0.6 | 4.7 | Kidney Margin | 12.0 | 40.9 |
| (ODO3868) | | | 9010321 | | |
| CC Mod Diff | 0.1 | 55.5 | Normal Uterus | 2.8 | 10.4 |
| (ODO3920) | | | | | |
| CC Margin | 1.1 | 14.2 | Uterine Cancer | 0.6 | 28.9 |
| (ODO3920) | | | 064011 | | |
| CC Gr.2 ascend | 0.1 | 62.9 | Normal Thyroid | 15.1 | 8.4 |
| colon (ODO3921) | | | | | |
| CC Margin | 0.6 | 12.1 | Thyroid Cancer | 7.1 | 16.7 |
| (ODO3921) | | | | | |
| CC from Partial | 0.3 | 41.5 | Thyroid Cancer | 0.9 | 24.7 |
| Hepatectomy | | | A302152 | | |
| (ODO4309) Mets | | | | | |
| Liver Margin | 2.4 | 13.6 | Thyroid Margin | 3.1 | 17.7 |
| (ODO4309) | | | A302153 | | |
| Colon mets to lung | 0.2 | 18.0 | Normal Breast | 0.3 | 60.3 |
| (OD04451-01) | | | | | |
| Lung Margin | 0.4 | 25.5 | Breast Cancer | 0.0 | 24.1 |
| (OD04451-02) | | | | | |
| Normal Prostate | 3.3 | 17.0 | Breast Cancer | 0.2 | 47.0 |
| 6546-1 | | | (OD04590-01) | | |
| Prostate Cancer | 3.4 | 33.7 | Breast Cancer | 0.7 | 72.7 |
| (OD04410) | | | Mets | | |
| | | | (OD04590-03) | | |
| Prostate Margin | 0.5 | 28.9 | Breast Cancer | 0.0 | 37.4 |
| (OD04410) | | | Metastasis | | |
| Prostate Cancer | 0.3 | 33.7 | Breast Cancer | 0.2 | 36.9 |
| (OD04720-01) | | | | | |
| Prostate Margin | 2.6 | 45.7 | Breast Cancer | 0.1 | 65.1 |
| (OD04720-02) | | | | | |
| Normal Lung | 0.7 | 80.7 | Breast Cancer | 0.4 | 39.8 |
| | | | 9100266 | | |
| Lung Met to | 0.3 | 100.0 | Breast Margin | 0.3 | 31.2 |
| Muscle | | | 9100265 | | |
| (ODO4286) | | | | | |
| Muscle Margin | 100.0 | 21.5 | Breast Cancer | 0.2 | 49.0 |
| (ODO4286) | | | A209073 | | |
| Lung Malignant | 0.3 | 57.8 | Breast Margin | 0.0 | 44.8 |
| Cancer | | | A2090734 | | |
| (OD03126) | | | | | |
| Lung Margin | 0.4 | 61.6 | Normal Liver | 1.6 | 4.5 |
| (OD03126) | | | | | |
| Lung Cancer | 0.1 | 70.2 | Liver Cancer | 0.9 | 2.6 |
| (OD04404) | | | | | |
| Lung Margin | 0.3 | 34.2 | Liver Cancer | 1.1 | 4.7 |
| (OD04404) | | | 1025 | | |
| Lung Cancer | 0.0 | 87.7 | Liver Cancer | 1.0 | 18.3 |
| (OD04565) | | | 1026 | | |
| Lung Margin | 0.8 | 23.8 | Liver Cancer | 2.3 | 7.6 |
| (OD04565) | | | 6004-T | | |
| Lung Cancer | 0.2 | 41.5 | Liver Tissue | 0.3 | 12.0 |
| (OD04237-01) | | | 6004-N | | |
| Lung Margin | 0.5 | 34.2 | Liver Cancer | 0.7 | 12.1 |
| (OD04237-02) | | | 6005-T | | |
| Ocular Mel Met to | 1.3 | 97.3 | Liver Tissue | 1.6 | 5.7 |
| Liver (ODO4310) | | | 6005-N | | |
| Liver Margin | 3.2 | 5.0 | Normal Bladder | 0.9 | 38.2 |
| (ODO4310) | | | | | |
| Melanoma | 0.0 | 87.7 | Bladder Cancer | 0.0 | 21.3 |
| Metastasis | | | | | |
| Lung Margin | 0.6 | 56.3 | Bladder Cancer | 0.1 | 46.0 |
| (OD04321) | | | | | |
| Normal Kidney | 18.8 | 30.1 | Bladder Cancer | 0.2 | 96.6 |
| | | | (OD04718-01) | | |
| Kidney Ca, | 7.5 | 46.7 | Bladder Normal | 2.9 | 29.5 |
| Nuclear grade 2 | | | Adjacent | | |
| (OD04338) | | | (OD04718-03) | | |
| Kidney Margin | 6.0 | 14.8 | Normal Ovary | 1.1 | 21.5 |
| (OD04338) | | | | | |
| Kidney Ca | 11.3 | 52.1 | Ovarian Cancer | 0.3 | 73.7 |
| Nuclear grade 1/2 | | | | | |
| (OD04339) | | | | | |
| Kidney Margin | 14.2 | 20.3 | Ovarian Cancer | 0.0 | 48.3 |
| (OD04339) | | | (OD04768-07) | | |
| Kidney Ca, Clear | 2.5 | 49.0 | Ovary Margin | 0.2 | 18.8 |
| cell type | | | (OD04768-08) | | |
| (OD04340) | | | | | |
| Kidney Margin | 11.4 | 23.2 | Normal | 0.9 | 13.9 |
| (OD04340) | | | Stomach | | |
| Kidney Ca, | 0.9 | 42.6 | Gastric Cancer | 0.3 | 6.7 |
| Nuclear grade 3 | | | 9060358 | | |
| (OD04348) | | | | | |
| Kidney Margin | 9.3 | 28.9 | Stomach | 0.3 | 13.2 |
| (OD04348) | | | Margin | | |
| | | | 9060359 | | |
| Kidney Cancer | 0.4 | 50.7 | Gastric Cancer | 1.3 | 28.3 |
| (OD04622-01) | | | 9060395 | | |
| Kidney Margin | 1.7 | 8.6 | Stomach | 0.4 | 18.0 |
| (OD04622-03) | | | Margin | | |
| | | | 9060394 | | |
| Kidney Cancer | 6.2 | 21.8 | Gastric Cancer | 0.4 | 45.4 |
| (OD04450-01) | | | 9060397 | | |
| Kidney Margin | 6.1 | 18.2 | Stomach | 0.0 | 10.4 |
| (OD04450-03) | | | Margin | | |
| | | | 9060396 | | |
| Kidney Cancer | 0.9 | 25.0 | Gastric Cancer | 0.5 | 48.3 |
| 8120607 | | | 064005 | | |

TABLE 53
Panel 4.1D

| | Rel. Exp. (%) | | Rel. Exp. (%) |
|---|---|---|---|
| | Ag2284, Run | | Ag2284, Run |
| Tissue Name | 170069125 | Tissue Name | 170069125 |
| Secondary Th1 act | 0.0 | HUVEC IL-1beta | 0.0 |
| Secondary Th2 act | 1.0 | HUVEC IFN gamma | 0.0 |
| Secondary Tr1 act | 0.0 | HUVEC TNF alpha + IFN | 0.0 |
| | | gamma | |
| Secondary Th1 rest | 0.7 | HUVEC TNF alpha + IL4 | 0.0 |
| Secondary Th2 rest | 0.5 | HUVEC IL-11 | 0.0 |
| Secondary Tr1 rest | 0.0 | Lung Microvascular EC none | 0.0 |
| Primary Th1 act | 0.0 | Lung Microvascular EC | 0.0 |
| | | TNFaIpha + IL-1beta | |
| Primary Th2 act | 0.7 | Microvascular Dermal EC | 0.0 |
| | | none | |
| Primary Tr1 act | 0.0 | Microsvasular Dermal EC | 0.0 |
| | | TNF alpha + IL-1beta | |
| Primary Th1 rest | 0.0 | Bronchial epithelium | 1.0 |
| | | TNF alpha + IL1beta | |
| Primary Th2 rest | 0.0 | Small airway epithelium | 0.0 |
| | | none | |
| Primary Tr1 rest | 0.0 | Small airway epithelium | 0.0 |
| | | TNF alpha + IL-1beta | |
| CD45RA CD4 | 7.5 | Coronery artery SMC rest | 0.0 |
| lymphocyte act | | | |
| CD45RO CD4 | 0.0 | Coronery artery SMC | 0.0 |
| lymphocyte act | | TNF alpha + IL-1beta | |
| CD8 lymphocyte act | 0.0 | Astrocytes rest | 1.9 |
| Secondary CD8 | 0.0 | Astrocytes TNF alpha + IL- | 3.2 |
| lymphocyte rest | | 1beta | |
| Secondary CD8 | 0.0 | KU-812 (Basophil) rest | 0.0 |
| lymphocyte act | | | |
| CD4 lymphocyte none | 0.0 | KU-812 (Basophil) | 0.9 |
| | | PMA/ionomycin | |
| 2ry Th1/Th2/Tr1_anti- | 1.2 | CCD1106 (Keratinocytes) | 0.0 |
| CD95 CH11 | | none | |
| LAK cells rest | 0.8 | CCD1106 (Keratinocytes) | 0.0 |
| | | TNF alpha + IL-1beta | |
| LAK cells IL-2 | 0.0 | Liver cirrhosis | 2.2 |
| LAK cells IL-2 + IL-12 | 0.4 | NCI-H292 none | 0.8 |
| LAK cells IL-2 + IFN | 0.0 | NCI-H292 IL-4 | 0.0 |
| gamma | | | |
| LAK cells IL-2 + IL-18 | 0.0 | NCI-H292 IL-9 | 0.0 |
| LAK cells | 1.5 | NCI-H292 IL-13 | 0.0 |
| PMA/ionomycin | | | |
| NK Cells IL-2 rest | 1.3 | NCI-H292 IFN gamma | 0.0 |
| Two Way MLR 3 day | 1.3 | HPAEC none | 0.0 |
| Two Way MLR 5 day | 1.8 | HPAEC TNF alpha + IL-1 | 0.0 |
| | | beta | |
| Two Way MLR 7 day | 0.0 | Lung fibroblast none | 27.9 |
| PBMC rest | 0.0 | Lung fibroblast TNF alpha + | 4.7 |
| | | IL-1beta | |
| PBMC PWM | 0.9 | Lung fibroblast IL-4 | 19.3 |
| PBMC PHA-L | 0.0 | Lung fibroblast IL-9 | 32.3 |
| Ramos (B cell) none | 0.0 | Lung fibroblast IL-13 | 11.4 |
| Ramos (B cell) | 0.0 | Lung fibroblast IFN gamma | 9.9 |
| ionomycin | | | |
| B lymphocytes PWM | 0.8 | Dermal fibroblast CCD1070 | 43.2 |
| | | rest | |
| B lymphocytes CD40L | 0.0 | Dermal fibroblast CCD1070 | 31.0 |
| and IL-4 | | TNF alpha | |
| EOL-1 dbcAMP | 0.0 | Dermal fibroblast CCD1070 | 7.4 |
| | | IL-1beta | |
| EOL-1 dbcAMP | 0.0 | Dermal fibroblast IFN | 5.8 |
| PMA/ionomycin | | gamma | |
| Dendritic cells none | 0.0 | Dermal fibroblast IL-4 | 38.4 |
| Dendritic cells LPS | 0.5 | Dermal Fibroblasts rest | 24.7 |
| Dendritic cells anti- | 0.9 | Neutrophils TNFa + LPS | 0.0 |
| CD40 | | | |
| Monocytes rest | 0.0 | Neutrophils rest | 0.0 |
| Monocytes LPS | 2.4 | Colon | 1.0 |
| Macrophages rest | 8.9 | Lung | 7.3 |
| Macrophages LPS | 0.0 | Thymus | 3.1 |
| HUVEC none | 0.0 | Kidney | 100.0 |
| HUVEC starved | 0.0 | | |

TABLE 54
Panel 4D

| | Rel. Exp. (%) | | Rel. Exp. (%) |
|---|---|---|---|
| | Ag2011, Run | | Ag2011, Run |
| Tissue Name | 160997385 | Tissue Name | 160997385 |
| Secondary Th1 act | 4.7 | HUVEC IL-1beta | 2.0 |
| Secondary Th2 act | 6.4 | HUVEC IFN gamma | 4.0 |
| Secondary Tr1 act | 8.6 | HUVEC TNF alpha + IFN | 5.0 |
| | | gamma | |
| Secondary Th1 rest | 0.6 | HUVEC TNF alpha + IL4 | 8.4 |
| Secondary Th2 rest | 1.7 | HUVEC IL-11 | 3.5 |
| Secondary Tr1 rest | 1.7 | Lung Microvascular EC none | 13.0 |
| Primary Th1 act | 14.0 | Lung Microvascular EC | 15.3 |
| | | TNF alpha + IL-1beta | |
| Primary Th2 act | 7.7 | Microvascular Dermal EC | 23.2 |
| | | none | |
| Primary Tr1 act | 12.9 | Microsvasular Dermal EC | 17.3 |
| | | TNF alpha + IL-1beta | |
| Primary Th1 rest | 3.3 | Bronchial epithelium | 4.5 |
| | | TNF alpha + IL-1beta | |
| Primary Th2 rest | 2.3 | Small airway epithelium | 16.0 |
| | | none | |
| Primary Tr1 rest | 2.0 | Small airway epithelium | 100.0 |
| | | TNF alpha + IL-1beta | |
| CD45RA CD4 | 6.5 | Coronery artery SMC rest | 15.7 |
| lymphocyte act | | | |
| CD45RO CD4 | 5.3 | Coronery artery SMC | 11.1 |
| lymphocyte act | | TNF alpha + IL-1beta | |
| CD8 lymphocyte act | 3.3 | Astrocytes rest | 25.3 |
| Secondary CD8 | 7.2 | Astrocytes TNF alpha + IL- | 21.6 |
| lymphocyte rest | | 1beta | |
| Secondary CD8 | 3.0 | KU-812 (Basophil) rest | 8.4 |
| lymphocyte act | | | |
| CD4 lymphocyte none | 1.6 | KU-812 (Basophil) | 39.5 |
| | | PMA/ionomycin | |
| 2ry Th1/Th2/Tr1_anti- | 0.3 | CCD1106 (Keratinocytes) | 35.1 |
| CD95 CH11 | | none | |
| LAK cells rest | 19.1 | CCD1106 (Keratinocytes) | 59 |
| | | TNF alpha + IL-1beta | |
| LAK cells IL-2 | 3.1 | Liver cirrhosis | 0.9 |
| LAK cells IL-2 + IL-12 | 6.5 | Lupus kidney | 1.3 |
| LAK cells IL-2 + IFN | 9.8 | NCI-H292 none | 42.3 |
| gamma | | | |
| LAK cells IL-2 + IL-18 | 5.9 | NCI-H292 IL-4 | 90.1 |
| LAK cells | 8.7 | NCI-H292 IL-9 | 58.2 |
| PMA/ionomycin | | | |
| NK Cells IL-2 rest | 1.7 | NCI-H292 IL-13 | 33.9 |
| Two Way MLR 3 day | 9.3 | NCI-H292 IFN gamma | 30.4 |
| Two Way MLR 5 day | 7.4 | HPAEC none | 5.8 |
| Two Way MLR 7 day | 2.0 | HPAEC TNF alpha + IL-1 | 12.9 |
| | | beta | |
| PBMC rest | 1.7 | Lung fibroblast none | 23.8 |
| PBMC PWM | 12.5 | Lung fibroblast TNF alpha + | 10.7 |
| | | IL-1beta | |
| PBMC PHA-L | 5.4 | Lung fibroblast IL-4 | 59.0 |
| Ramos (B cell) none | 0.5 | Lung fibroblast IL-9 | 40.6 |
| Ramos (B cell) | 0.9 | Lung fibroblast IL-13 | 31.0 |
| ionomycin | | | |
| B lymphocytes PWM | 15.6 | Lung fibroblast IFN gamma | 65.5 |
| B lymphocytes CD40L | 5.8 | Dermal fibroblast CCD1070 | 37.4 |
| and IL-4 | | rest | |
| EOL-1 dbcAMP | 3.5 | Dermal fibroblast CCD1070 | 50.0 |
| | | TNF alpha | |
| EOL-1 dbcAMP | 60.3 | Dermal fibroblast CCD1070 | 19.6 |
| PMA/ionomycin | | IL-1beta | |
| Dendritic cells none | 17.6 | Dermal fibroblast IFN | 15.0 |
| | | gamma | |
| Dendritic cells LPS | 32.5 | Dermal fibroblast IL-4 | 43.8 |
| Dendritic cells anti- | 21.0 | IBD Colitis 2 | 0.3 |
| CD40 | | | |
| Monocytes rest | 0.1 | IBD Crohn's | 0.8 |
| Monocytes LPS | 8.4 | Colon | 5.3 |
| Macrophages rest | 34.2 | Lung | 15.0 |
| Macrophages LPS | 11.3 | Thymus | 5.8 |
| HUVEC none | 6.5 | Kidney | 11.4 |
| HUVEC starved | 9.3 | | |

Panel 1.2 Summary: Ag1508 The expression of the NOV6 gene is highest in a sample derived from skeletal muscle (CT=19.5). Thus, this gene could be used to distinguish skeletal muscle from other tissues. Expression of the NOV6 gene is also high in kidney (CT=23).

The NOV6 gene is also moderately expressed in other metabolically relevant tissues including heart, adrenal gland, pancreas, thyroid, pituitary gland, and liver (CT values from 29-32). The widespread expression of the NOV6 gene in tissues with metabolic function suggests a role in metabolic disorders such as obesity and diabetes.

The NOV6 gene is moderately expressed in the brain in at least the thalamus, hippocampus, cerebellum, amygdala and is highly expressed in the cerebral cortex, suggesting that this gene product has functional significance in the CNS. Please see Panel 1.3D for potential utility of this gene in the central nervous system.

Panel 1.3D Summary: Ag1586/2011/Ag2284 Two experiments with the same probe and primer set produce results that are in excellent agreement. The NOV6 gene appears to be expressed largely in cancer cell lines, with highest expression in a melanoma cell line (CTs=26-28). Of note is the expression associated with colon cancer cell lines and melanoma cell lines. Thus, the expression of thie gene could be used to distinguish these samples from other samples on the panel. Moreover, therapeutic modulation of this gene, through the use of small molecule drugs, antibodies or protein therapeutics might be of use in the treatment of colon cancer or melanoma.

The NOV6 gene is modestly expressed (CT values=31-34) in a variety of metabolic tissues including pancreas, adrenal, thyroid, pituitary, fetal liver, and adipose. Thus, this gene product may be an antibody target for the treatment of metabolic disease, including obesity and diabetes, in any or all of these tissues. Furthermore, the NOV6 is expressed at higher levels in fetal (CT values=26-28) versus adult heart (CT values=31-33), and in fetal (CT values=26-28) versus adult skeletal muscle (CT values=32-33), and may be used to differentiate between the adult and fetal sources of these tissues. Furthermore, the higher levels of expression in the fetal tissues suggest that the NOV6 gene product may be involved in the development of heart and skeletal muscle tissue. Thus, therapeutic modulation of the expression or function of the protein encoded by the NOV6 gene may be beneficial in the treatment of diseases that result in weak or dystrophic heart or skeletal muscle tissue, including ardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, muscular dystrophy, Lesch-Nyhan syndrome, and myasthenia gravis.

This gene represents a novel protein with homology to a plexin that is expressed at moderate to high levels in all brain regions examined. Plexins act as receptors for semaphorins in the CNS. The interactions of the semaphorins and their receptors are critical for axon guidance. Therefore, this gene product may be useful as a drug target in clinical conditions where axonal growth and/or compensatory synaptogenesis are desireable (spinal cord or head trauma, stroke, or neurodegenerative diseases such as Alzheimer's, Parkinson's, or Huntington's disease).

References:

1. Pasterkamp R J, Ruitenberg M J, Verhaagen J. Semaphorins and their receptors in olfactory axon guidance. Cell Mol Biol (Noisy-le-grand) September 1999; 45(6):763-79

The mammalian olfactory system is capable of discriminating among a large variety of odor molecules and is therefore essential for the identification of food, enemies and mating partners. The assembly and maintenance of olfactory connectivity have been shown to depend on the combinatorial actions of a variety of molecular signals, including extracellular matrix, cell adhesion and odorant receptor molecules. Recent studies have identified semaphorins and their receptors as putative molecular cues involved in olfactory pathfinding, plasticity and regeneration. The semaphorins comprise a large family of secreted and transmembrane axon guidance proteins, being either repulsive or attractive in nature. Neuropilins were shown to serve as receptors for secreted class 3 semaphorins, whereas members of the plexin family are receptors for class 1 and V (viral) semaphorins. The present review will discuss a role for semaphorins and their receptors in the establishment and maintenance of olfactory connectivity.

2. Murakami Y, Suto F, Shimizu M, Shinoda T, Kameyama T, Fujisawa H. Differential expression of plexin-A subfamily members in the mouse nervous system. Dev Dyn March 2001; 220(3):246-58

Plexins comprise a family of transmembrane proteins (the plexin family) which are expressed in nervous tissues. Some plexins have been shown to interact directly with secreted or transmembrane semaphorins, while plexins belonging to the A subfamily are suggested to make complexes with other membrane proteins, neuropilins, and propagate chemorepulsive signals of secreted semaphorins of class 3 into cells or neurons. Despite that much information has been gathered on the plexin-semaphorin interaction, the role of plexins in the nervous system is not well understood. To gain insight into the functions of plexins in the nervous system, we analyzed spatial and temporal expression patterns of three members of the plexin-A subfamily (plexin-A1, -A2, and -A3) in the developing mouse nervous system by in situ hybridization analysis in combination with immunohistochemistry. We show that the three plexins are differentially expressed in sensory receptors or neurons in a developmentally regulated manner, suggesting that a particular plexin or set of plexins is shared by neuronal elements and functions as the receptor for semaphorins to regulate neuronal development.

Panel 2.2 Summary: Ag2011 The expression of thie gene appears to be highest in a sample derived from a melanoma metastasis. In addition, there is substantial expression in another melanoma sample. This expression is concordant with the expression detected in Panel 1.3D. Thus, the expression of this gene could be used to distinguish melanoma from other cancer types in this panel. Moreover, therapeutic modulation of this gene, through the use of small molecule drugs, antibodies or protein therapeutics might be of use in the treatment of melanoma.

Panel 2D Summary: Ag1508/Ag1586 Expression of the SC126413398_A gene in this panel is highest in a sample of muscle tissue adjacent to a metastatic cancer and in a metastasis of lung cancer.

Panel 4.1D Summary: Ag2284 Significant expression in this panel is limited to kidney. This observation is consistent with what was observed in other panels. Therefore, therapuetic drugs designed against the SC126413398_A gene product may be important for regulating the function of the kidney.

Panel 4D Summary: Ag2011 Significant expression of this transcript is found in small airway epithelium upon treatment with the pro-inflammatory cytokines TNF-a and IL-1b (CT=26.5), the muco-epidermoid cell line H 292 treated with IL-4 or IL-9, and in lung fibroblasts treated with IFN-g or IL-4. The constitutive expression of this transcript in these tissues is highly up-regulated by pro-inflammatory cytokines or in conditions reflecting a Th2 mediated mechanism. Therefore, modulation of the expression of the protein encoded by this transcript could be useful for the treatment of lung inflammatory diseases that result from infection of the lung (bronchitis, pneumonia) and for the treatment of Th2 -mediated lung disease such as asthma or COPD. Significant expression of this transcript is also found in eosinophils upon PMA and ionomycin treatment, conditions that lead to production of eosinophil specific mediators. This production could contribute to the pathologies associated with asthma, other atopic diseases and inflammatory bowel disease. This gene encodes a novel protein with homology to members of the plexin family, a family of transmembrane proteins which act as receptors for semaphorins. In neurons, semaphorins provide essential attractive and repulsive cues that are necessary for axon guidance. The description of the interaction of plexin wih tyrosine kinase in the fetal lung suggests that this protein may play a role not only in morphogenesis but also in proliferation of activation. (See reference below.) Therefore, modulation of the experession of this protein by either antibody or small molecules could be beneficial for the treatment of inflammatory lung, bowel and skin diseases.

Reference:

1. Cell Oct. 1, 1999; 99(1):71-80

Plexins are a large family of receptors for transmembrane, secreted, and GPI-anchored semaphorins in vertebrates.

Tamagnone L, Artigiani S, Chen H, He Z, Ming G I, Song H, Chedotal A, Winberg M L, Goodman C S, Poo M, Tessier-Lavigne M, Comoglio P M.

Institute for Cancer Research and Treatment, University of Torino, Candiolo, Italy. Itamagnone@ircc.unito.it

In Drosophila, plexin A is a functional receptor for semaphorin-1a. Here we show that the human plexin gene family comprises at least nine members in four subfamilies. Plexin-B1 is a receptor for the transmembrane semaphorin Sema4D (CD 100), and plexin-C1 is a receptor for the GPI-anchored semaphorin Sema7A (Sema-K1). Secreted (class 3) semaphorins do not bind directly to plexins, but rather plexins associate with neuropilins, coreceptors for these semaphorins. Plexins are widely expressed: in neurons, the expression of a truncated plexin-A1 protein blocks axon repulsion by Sema3A. The cytoplasmic domain of plexins associates with a tyrosine kinase activity. Plexins may also act as ligands mediating repulsion in epithelial cells in vitro. We conclude that plexins are receptors for multiple (and perhaps all) classes of semaphorins, either alone or in combination with neuropilins, and trigger a novel signal transduction pathway controlling cell repulsion

PMID: 10520995

NOV7

Expression of gene NOV7 was assessed using the primer-probe sets Ag2262 and Ag2316, described in Tables 55 and 56. Results of the RTQ-PCR runs are shown in Tables 57, 58and59.

TABLE 55

Probe Name Ag2262

| | | | Start | SEQ ID |
|---|---|---|---|---|
| Primers | Sequences | Length | Position | NO: |
| Forward | 5′-gacctggtgtacatggagga-3′ | 20 | 761 | 172 |
| Probe | TET-5′-cttctgccggcccagcaagtact-3′-TAMRA | 23 | 790 | 173 |
| Reverse | 5′-gagcacaccctacctgctg-3′ | 19 | 822 | 174 |

TABLE 56

Probe Name Ag2316

| | | | | |
|---|---|---|---|---|
| | | | | SEQ |
| | | | Start | ID |
| Primers | Sequences | Length | Position | NO: |

| | | | | |
|---|---|---|---|---|
| Forward | 5′-gtccaagagaggaaacaagga | 21 | 457 | 175 |
| | -3′ | | | |
| Probe | TET-5′-cacaatacccacgtgg- | 24 | 500 | 176 |
| | gcatcaag-3′-TAMRA | | | |
| Reverse | 5′-gtcctgaggccactcttcac- | 20 | 527 | 177 |
| | 3′ | | | |

TABLE 57
Panel 1.3D

| | Rel. | Rel. | Rel. | | Rel. | Rel. | Rel. |
|---|---|---|---|---|---|---|---|
| | Exp. (%) | Exp. (%) | Exp. (%) | | Exp. (%) | Exp. (%) | Exp. (%) |
| | Ag2262, | Ag2262, | Ag2316, | | Ag2262, | Ag2262, | Ag2316, |
| | Run | Run | Run | Tissue | Run | Run | Run |
| Tissue Name | 150719071 | 167966858 | 162185396 | Name | 150719071 | 167966858 | 162185396 |
| Liver | 0.0 | 6.7 | 0.0 | Kidney | 24.0 | 100.0 | 50.0 |
| adenocarcinoma | | | | (fetal) | | | |
| Pancreas | 0.0 | 0.0 | 0.0 | Renal ca. | 0.0 | 0.0 | 0.0 |
| | | | | 786-0 | | | |
| Pancreatic ca. | 0.0 | 0.0 | 0.0 | Renal ca. | 0.0 | 12.6 | 0.0 |
| CAPAN 2 | | | | A498 | | | |
| Adrenal gland | 1.9 | 0.0 | 0.0 | Renal ca. | 0.0 | 0.0 | 0.0 |
| | | | | RXF 393 | | | |
| Thyroid | 2.2 | 0.0 | 0.0 | Renal ca. | 0.0 | 0.0 | 0.0 |
| | | | | ACHN | | | |
| Salivary gland | 0.3 | 0.0 | 0.0 | Renal ca. | 0.2 | 0.0 | 0.0 |
| | | | | UO-31 | | | |
| Pituitary gland | 0.0 | 8.0 | 0.0 | Renal ca. | 0.0 | 0.0 | 0.0 |
| | | | | TK-10 | | | |
| Brain (fetal) | 0.0 | 1.0 | 0.0 | Liver | 0.0 | 0.0 | 0.0 |
| Brain (whole) | 5.2 | 0.0 | 26.2 | Liver (fetal) | 0.0 | 0.0 | 0.0 |
| Brain | 6.8 | 3.8 | 11.5 | Liver ca. | 0.0 | 0.0 | 0.0 |
| (amygdala) | | | | (hepatoblast) | | | |
| | | | | HepG2 | | | |
| Brain | 1.0 | 6.4 | 0.0 | Lung | 6.8 | 0.0 | 19.3 |
| (cerebellum) | | | | | | | |
| Brain | 16.5 | 0.0 | 0.0 | Lung (fetal) | 8.5 | 0.0 | 6.8 |
| (hippocampus) | | | | | | | |
| Brain | 2.0 | 0.0 | 0.0 | Lung ca. | 0.0 | 0.0 | 0.0 |
| (substantia | | | | (small cell) | | | |
| nigra) | | | | LX-1 | | | |
| Brain | 4.9 | 11.2 | 57.0 | Lung ca. | 0.3 | 0.0 | 0.0 |
| (thalamus) | | | | (small cell) | | | |
| | | | | NCI-H69 | | | |
| Cerebral Cortex | 2.5 | 13.3 | 3.3 | Lung ca. | 2.5 | 6.9 | 0.0 |
| | | | | (s.cell var.) | | | |
| | | | | SHP-77 | | | |
| Spinal cord | 3.3 | 9.2 | 6.8 | Lung ca. | 0.0 | 0.0 | 0.0 |
| | | | | (large | | | |
| | | | | cell)NCI- | | | |
| | | | | H460 | | | |
| glio/astro U87- | 0.0 | 0.0 | 0.0 | Lung ca. | 0.0 | 6.4 | 0.0 |
| MG | | | | (non-sm. | | | |
| | | | | cell) A549 | | | |
| glio/astro U- | 0.0 | 0.0 | 0.0 | Lung ca. | 0.0 | 0.0 | 0.0 |
| 118-MG | | | | (non-s.cell) | | | |
| | | | | NCI-H23 | | | |
| astrocytoma | 0.0 | 0.0 | 0.0 | Lung ca. | 0.0 | 0.0 | 0.0 |
| SW1783 | | | | (non-s.cell) | | | |
| | | | | HOP-62 | | | |
| neuro*; met | 0.0 | 0.0 | 0.0 | Lung ca. | 2.8 | 0.0 | 0.0 |
| SK-N-AS | | | | (non-s.cl) | | | |
| | | | | NCI-H522 | | | |
| astrocytoma SF- | 0.0 | 0.0 | 0.0 | Lung ca. | 0.0 | 0.0 | 0.0 |
| 539 | | | | (squam.) SW | | | |
| | | | | 900 | | | |
| astrocytoma | 0.0 | 0.0 | 0.0 | Lung ca. | 0.0 | 0.0 | 0.0 |
| SNB-75 | | | | (squam.) | | | |
| | | | | NCI-H596 | | | |
| glioma SNB-19 | 0.0 | 0.0 | 0.0 | Mammary | 0.0 | 0.0 | 0.0 |
| | | | | gland | | | |
| glioma U251 | 0.0 | 0.0 | 0.0 | Breast ca.* | 0.0 | 0.0 | 0.0 |
| | | | | (pl.ef) | | | |
| | | | | MCF-7 | | | |
| glioma SF-295 | 0.0 | 0.0 | 0.0 | Breast ca.* | 0.0 | 0.0 | 0.0 |
| | | | | (pl.ef) | | | |
| | | | | MDA-MB- | | | |
| | | | | 231 | | | |
| Heart (Fetal) | 2.0 | 0.0 | 33.4 | Breast ca.* | 0.0 | 0.0 | 0.0 |
| | | | | (pl.ef) | | | |
| | | | | T47D | | | |
| Heart | 0.0 | 6.7 | 9.6 | Breast ca. | 0.0 | 0.0 | 0.0 |
| | | | | BT-549 | | | |
| Skeletal muscle | 2.5 | 0.0 | 8.2 | Breast ca. | 1.0 | 0.0 | 0.0 |
| (Fetal) | | | | MDA-N | | | |
| Skeletal muscle | 0.0 | 0.0 | 0.0 | Ovary | 0.0 | 0.0 | 6.4 |
| Bone marrow | 0.9 | 0.0 | 0.0 | Ovarian ca. | 0.0 | 0.0 | 0.0 |
| | | | | OVCAR-3 | | | |
| Thymus | 0.0 | 0.0 | 0.0 | Ovarian ca. | 0.0 | 0.0 | 0.0 |
| | | | | OVCAR-4 | | | |
| Spleen | 100.0 | 65.5 | 100.0 | Ovarian ca. | 0.0 | 0.0 | 0.0 |
| | | | | OVCAR-5 | | | |
| Lymph node | 0.0 | 0.0 | 0.0 | Ovarian ca. | 0.0 | 0.0 | 0.0 |
| | | | | OVCAR-8 | | | |
| Colorectal | 10.8 | 19.8 | 0.0 | Ovarian ca. | 0.0 | 0.0 | 0.0 |
| | | | | IGROV-1 | | | |
| Stomach | 2.7 | 0.0 | 0.0 | Ovarian ca. | 0.0 | 0.0 | 0.0 |
| | | | | (ascites) SK- | | | |
| | | | | OV-3 | | | |
| Small intestine | 6.4 | 0.0 | 0.0 | Uterus | 0.0 | 0.0 | 0.0 |
| Colon ca. | 0.0 | 0.0 | 0.0 | Placenta | 0.6 | 7.1 | 0.0 |
| SW480 | | | | | | | |
| Colon ca.* | 1.2 | 0.0 | 0.0 | Prostate | 0.0 | 1.8 | 4.9 |
| SW620 (SW480 | | | | | | | |
| met) | | | | | | | |
| Colon ca. HT29 | 0.0 | 0.0 | 0.0 | Prostate ca.* | 0.0 | 0.0 | 0.0 |
| | | | | (bone met) | | | |
| | | | | PC-3 | | | |
| Colon ca. HCT- | 0.0 | 0.0 | 0.0 | Testis | 1.7 | 0.0 | 7.2 |
| 116 | | | | | | | |
| Colon ca. | 2.5 | 6.6 | 0.0 | Melanoma | 0.0 | 0.0 | 0.0 |
| CaCo-2 | | | | Hs688(A).T | | | |
| CC Well to | 0.0 | 0.0 | 0.0 | Melanoma* | 0.0 | 0.0 | 0.0 |
| Mod Diff | | | | (met) | | | |
| (ODO3866) | | | | Hs688(B).T | | | |
| Colon ca. HCC- | 0.0 | 0.0 | 0.0 | Melanoma | 0.0 | 0.0 | 0.0 |
| 2998 | | | | UACC-62 | | | |
| Gastric ca. | 0.0 | 14.7 | 0.0 | Melanoma | 0.0 | 0.0 | 0.0 |
| (liver met) NCI- | | | | M14 | | | |
| N87 | | | | | | | |
| Bladder | 0.0 | 6.5 | 16.2 | Melanoma | 0.0 | 0.0 | 0.0 |
| | | | | LOX IMVI | | | |
| Trachea | 5.0 | 0.0 | 6.0 | Melanoma* | 0.0 | 0.0 | 0.0 |
| | | | | (met) SK- | | | |
| | | | | MEL-5 | | | |
| Kidney | 14.9 | 7.9 | 31.0 | Adipose | 0.0 | 0.0 | 7.6 |

TABLE 58
Panel 2D

| | Rel. Exp. (%) | | Rel. Exp. (%) |
|---|---|---|---|
| | Ag2262, Run | | Ag2262, Run |
| Tissue Name | 150943107 | Tissue Name | 150943107 |
| Normal Colon | 14.2 | Kidney Margin | 24.0 |
| | | 8120608 | |
| CC Well to Mod Diff | 14.2 | Kidney Cancer 8120613 | 0.0 |
| (ODO3866) | | | |
| CC Margin (ODO3866) | 0.0 | Kidney Margin | 46.3 |
| | | 8120614 | |
| CC Gr.2 rectosigmoid | 0.0 | Kidney Cancer 9010320 | 0.0 |
| (ODO3868) | | | |
| CC Margin (ODO3868) | 0.0 | Kidney Margin | 16.5 |
| | | 9010321 | |
| CC Mod Diff (ODO3920) | 0.0 | Normal Uterus | 16.4 |
| CC Margin (ODO3920) | 0.8 | Uterine Cancer 064011 | 0.0 |
| CC Gr.2 ascend colon | 0.0 | Normal Thyroid | 15.6 |
| (ODO3921) | | | |
| CC Margin (ODO3921) | 0.9 | Thyroid Cancer | 0.0 |
| CC from Partial | 0.0 | Thyroid Cancer | 6.8 |
| Hepatectomy (ODO4309) | | A302152 | |
| Mets | | | |
| Liver Margin (ODO4309) | 1.1 | Thyroid Margin | 0.0 |
| | | A302153 | |
| Colon mets to lung | 7.3 | Normal Breast | 9.3 |
| (OD04451-01) | | | |
| Lung Margin (OD04451-02) | 0.0 | Breast Cancer | 0.0 |
| Normal Prostate 6546-1 | 18.6 | Breast Cancer | 4.8 |
| | | (OD04590-01) | |
| Prostate Cancer (OD04410) | 10.2 | Breast Cancer Mets | 8.5 |
| | | (OD04590-03) | |
| Prostate Margin (OD04410) | 0.0 | Breast Cancer | 0.0 |
| | | Metastasis | |
| Prostate Cancer (OD04720- | 0.0 | Breast Cancer | 7.2 |
| 01) | | | |
| Prostate Margin (OD04720- | 9.8 | Breast Cancer | 0.0 |
| 02) | | | |
| Normal Lung | 22.5 | Breast Cancer 9100266 | 0.7 |
| Lung Met to Muscle | 6.1 | Breast Margin 9100265 | 0.0 |
| (ODO4286) | | | |
| Muscle Margin (ODO4286) | 0.0 | Breast Cancer A209073 | 0.0 |
| Lung Malignant Cancer | 5.4 | Breast Margin | 0.0 |
| (OD03126) | | A2090734 | |
| Lung Margin (OD03126) | 0.0 | Normal Liver | 0.0 |
| Lung Cancer (OD04404) | 7.6 | Liver Cancer | 0.0 |
| Lung Margin (OD04404) | 3.8 | Liver Cancer 1025 | 5.6 |
| Lung Cancer (OD04565) | 0.0 | Liver Cancer 1026 | 2.4 |
| Lung Margin (OD04565) | 0.0 | Liver Cancer 6004-T | 0.0 |
| Lung Cancer (OD04237-01) | 0.0 | Liver Tissue 6004-N | 8.7 |
| Lung Margin (OD04237-02) | 6.9 | Liver Cancer 6005-T | 0.0 |
| Ocular Mel Met to Liver | 1.1 | Liver Tissue 6005-N | 0.0 |
| (ODO4310) | | | |
| Liver Margin (ODO4310) | 28.5 | Normal Bladder | 0.0 |
| Melanoma Metastasis | 0.0 | Bladder Cancer | 0.0 |
| Lung Margin (OD04321) | 0.0 | Bladder Cancer | 18.3 |
| Normal Kidney | 100.0 | Bladder Cancer | 0.0 |
| | | (OD04718-01) | |
| Kidney Ca, Nuclear grade 2 | 15.2 | Bladder Normal | 0.0 |
| (OD04338) | | Adjacent (OD04718-03) | |
| Kidney Margin (OD04338) | 40.3 | Normal Ovary | 0.0 |
| Kidney Ca Nuclear grade | 0.0 | Ovarian Cancer | 7.5 |
| 1/2 (OD04339) | | | |
| Kidney Margin (OD04339) | 50.0 | Ovarian Cancer | 0.0 |
| | | (OD04768-07) | |
| Kidney Ca, Clear cell type | 0.0 | Ovary Margin | 0.0 |
| (OD04340) | | (OD04768-08) | |
| Kidney Margin (OD04340) | 31.2 | Normal Stomach | 13.8 |
| Kidney Ca, Nuclear grade 3 | 0.0 | Gastric Cancer 9060358 | 0.0 |
| (OD04348) | | | |
| Kidney Margin (OD04348) | 29.9 | Stomach Margin | 0.0 |
| | | 9060359 | |
| Kidney Cancer (OD04622- | 0.0 | Gastric Cancer 9060395 | 0.0 |
| 01) | | | |
| Kidney Margin (OD04622- | 58.6 | Stomach Margin | 0.0 |
| 03) | | 9060394 | |
| Kidney Cancer (OD04450- | 0.0 | Gastric Cancer 9060397 | 0.0 |
| 01) | | | |
| Kidney Margin (OD04450- | 95.9 | Stomach Margin | 0.0 |
| 03) | | 9060396 | |
| Kidney Cancer 8120607 | 0.0 | Gastric Cancer 064005 | 0.0 |

TABLE 59
Panel 4D

| | Rel. Exp. (%) | Rel. Exp. (%) | | Rel. Exp. (%) | Rel. Exp. (%) |
|---|---|---|---|---|---|
| | Ag2262, Run | Ag2316, Run | | Ag2262, Run | Ag2316, Run |
| Tissue Name | 150981162 | 164037437 | Tissue Name | 150981162 | 164037437 |
| Secondary Th1 act | 0.0 | 0.0 | HUVEC IL-1beta | 0.0 | 0.0 |
| Secondary Th2 act | 0.0 | 0.0 | HUVEC IFN | 0.0 | 0.0 |
| | | | gamma | | |
| Secondary Tr1 act | 0.0 | 0.0 | HUVEC TNF alpha + | 11.6 | 0.0 |
| | | | IFN gamma | | |
| Secondary Th1 rest | 0.0 | 0.0 | HUVEC TNF alpha + | 0.0 | 0.0 |
| | | | IL4 | | |
| Secondary Th2 rest | 0.0 | 0.0 | HUVEC IL-11 | 8.7 | 0.0 |
| Secondary Tr1 rest | 0.0 | 0.0 | Lung Microvascular | 0.0 | 0.0 |
| | | | EC none | | |
| Primary Th1 act | 0.0 | 0.0 | Lung Microvascular | 0.0 | 0.0 |
| | | | EC TNF alpha + IL- | | |
| | | | 1 beta | | |
| Primary Th2 act | 0.0 | 0.0 | Microvascular | 0.0 | 0.0 |
| | | | Dermal EC none | | |
| Primary Tr1 act | 1.8 | 0.0 | Microsvasular | 0.0 | 0.0 |
| | | | Dermal EC | | |
| | | | TNF alpha + IL- | | |
| | | | 1 beta | | |
| Primary Th1 rest | 0.0 | 0.0 | Bronchial | 0.0 | 0.0 |
| | | | epithelium | | |
| | | | TNF alpha + | | |
| | | | IL1 beta | | |
| Primary Th2 rest | 0.0 | 0.0 | Small airway | 0.0 | 0.0 |
| | | | epithelium none | | |
| Primary Tr1 rest | 0.0 | 0.0 | Small airway | 0.0 | 0.0 |
| | | | epithelium | | |
| | | | TNF alpha + IL- | 0.0 | 0.0 |
| | | | 1 beta | | |
| CD45RA CD4 | 0.0 | 0.0 | Coronery artery | 0.0 | 0.0 |
| lymphocyte act | | | SMC rest | | |
| CD45RO CD4 | 0.0 | 0.0 | Coronery artery | 0.0 | 0.0 |
| lymphocyte act | | | SMC TNF alpha + | | |
| | | | IL-1 beta | | |
| CD8 lymphocyte act | 0.0 | 0.0 | Astrocytes rest | 0.0 | 0.0 |
| Secondary CD8 | 0.0 | 0.0 | Astrocytes | 0.0 | 0.0 |
| lymphocyte rest | | | TNF alpha + IL- | | |
| | | | 1 beta | | |
| Secondary CD8 | 0.0 | 0.0 | KU-812 (Basophil) | 0.0 | 25.3 |
| lymphocyte act | | | rest | | |
| CD4 lymphocyte | 0.0 | 0 0 | KU-812 (Basophil) | 0.0 | 0 0 |
| none | | | PMA/ionomycin | | |
| 2ry | 0.0 | 0.0 | CCD1106 | 0.0 | 0 0 |
| Th1/Th2/Tr1_anti- | | | (Kerationcytes) | | |
| CD95 CH11 | | | none | | |
| LAK cells rest | 0.0 | 00 | CCD1106 | 0.0 | 0.0 |
| | | | (Keratinocytes) | | |
| | | | TNF alpha + IL- | | |
| | | | 1 beta | | |
| LAK cells IL-2 | 0.0 | 0.0 | Liver cirrhosis | 0.0 | 0.0 |
| LAK cells IL-2+IL- | 0.0 | 0.0 | Lupus kidney | 0.0 | 21.9 |
| 12 | | | | | |
| LAK cells IL-2+IFN | 17.3 | 0.0 | NCI-H292 none | 0.0 | 0.0 |
| gamma | | | | | |
| LAK cells IL-2+ IL- | 0.0 | 0.0 | NCI-H292 IL-4 | 0.0 | 0.0 |
| 18 | | | | | |
| LAK cells | 0.0 | 0.0 | NCI-H292 IL-9 | 0.0 | 0.0 |
| PMA/ionomycin | | | | | |
| NK Cells IL-2 rest | 0.0 | 0.0 | NCI-H292 IL-13 | 0.0 | 0.0 |
| Two Way MLR 3 | 0.0 | 0.0 | NCI-H292 IFN | 0.0 | 0.0 |
| day | | | gamma | | |
| Two Way MLR 5 | 17.1 | 0.0 | HPAEC none | 0.0 | 0.0 |
| day | | | | | |
| Two Way MLR 7 | 0.0 | 0.0 | HPAEC TNF alpha + | 1.3 | 0.0 |
| day | | | IL-1 beta | | |
| PBMC rest | 0.0 | 00 | Lung fibroblast | 0.0 | 0.0 |
| | | | none | | |
| PBMC PWM | 0.0 | 0.0 | Lung fibroblast | 0.0 | 0.0 |
| | | | TNF alpha + IL-1 | | |
| | | | beta | | |
| PBMC PHA-L | 0.0 | 0.0 | Lung fibroblast IL-4 | 0.0 | 0.0 |
| Ramos (B cell) none | 0.0 | 0.0 | Lung fibroblast IL-9 | 0.0 | 0.0 |
| Ramos (B cell) | 0.0 | 0.0 | Lung fibroblast IL- | 0.0 | 0.0 |
| ionomycin | | | 13 | | |
| B lymphocytes | 0.0 | 0.0 | Lung fibroblast IFN | 0.0 | 0.0 |
| PWM | | | gamma | | |
| B lymphocytes | 0.0 | 0.0 | Dermal fibroblast | 0.0 | 0.0 |
| CD40L and IL-4 | | | CCD1070 rest | | |
| EOL-1 dbcAMP | 0.0 | 0.0 | Dermal fibroblast | 0.0 | 0.0 |
| | | | CCD1070 TNF | | |
| | | | alpha | | |
| EOL-1 dbcAMP | 0.0 | 0.0 | Dermal fibroblast | 0.0 | 0.0 |
| PMA/ionomycin | | | CCD1070 IL-1 beta | | |
| Dendritic cells none | 0.0 | 0.0 | Dermal fibroblast | 0.0 | 0.0 |
| | | | IFN gamma | | |
| Dendritic cells LPS | 2.9 | 0.0 | Dermal fibroblast | 0.0 | 0.0 |
| | | | IL-4 | | |
| Dendritic cells anti- | 0.0 | 0.0 | IBD Colitis 2 | 0.0 | 0.0 |
| CD40 | | | | | |
| Monocytes rest | 0.0 | 0.0 | IBD Crohn's | 0.0 | 0.0 |
| Monocytes LPS | 0.0 | 0.0 | Colon | 100.0 | 12.7 |
| Macrophages rest | 8.2 | 0.0 | Lung | 72.2 | 0.0 |
| Macrophages LPS | 0.0 | 0.0 | Thymus | 47.3 | 100.0 |
| HUVEC none | 0.0 | 0.0 | Kidney | 0.0 | 0.0 |
| HUVEC starved | 1.8 | 0.0 | | | |

CNS_neurodegeneration_v1.0 Summary: Ag2316 Data from this one run is not included due to a potential problem in one of the sample wells.

Panel 1.3D Summary: Ag2262/2316 The expression of this gene was assessed in 3 separate runs using two independent probe and primer sets with significant expression detected in spleen and fetal kidney in all runs. Thus, the expression of this gene could be used to distinguish spleen from other tissues in the panel. Moreover, the expression of this gene could also be used to distinguish fetal kidney tissue from adult kidney tissue.

Panel 2D Summary: Ag2262 The expression of this gene is highest in a sample derived from normal kidney tissue. Of note was the profound association of the expression of this gene with normal kidney tissue when compared to adjacent malignant tissue. Thus, the expression of this gene could be used to distinguish normal kidney tissue from malignant kidney tissue. Moreover, therapeutic modulation of the expression or function of this gene through the use of small molecule drugs, antibodies or protein therapeutics might be of benefit in the treatment of kidney cancer.

Panel 4D Summary: Ag2316 This transcript is expressed almost exclusively in the thymus (CT 33.2). Therefore, this transcript could be used for detection of thymic tissues.

Ag 2262 Using a second set of primers, expression of the NOV7 gene is also found in colon and lung, in addition to its expression in the thymus. Thus, this putative Wnt-15 protein may also play an important role in the normal homeostasis of these tissues. Therefore, therapeutics designed with the protein encoded by this transcript could be important for maintaining or restoring normal function to these organs during inflammation.

NOV8

Expression of gene NOV8 was assessed using the primer-probe set Ag2261, described in Table 60. Results of the RTQ-PCR runs are shown in Tables 61, 62 and 63.

TABLE 60

Probe Name Ag2261

| | | | | |
|---|---|---|---|---|
| | | | | SEQ |
| | | | Start | ID |
| Primers | Sequences | Length | Position | NO: |

| | | | | |
|---|---|---|---|---|
| Forward | 5′-ggatgactcgcctagcttct- | 20 | 858 | 178 |
| | 3′ | | | |
| Probe | TET-5′-gccgtaggtgcaccgt- | 23 | 911 | 179 |
| | gagaag-3′-TAMRA | | | |
| Reverse | 5′-agcagatgctctcgcagtt- | 19 | 934 | 180 |
| | 3′ | | | |

TABLE 61
Panel 1.3D

| | Rel. Exp. (%) | Rel. Exp. (%) | | Rel. Exp. (%) | Rel. Exp. (%) |
|---|---|---|---|---|---|
| | Ag2261, Run | Ag2261, Run | | Ag2261, Run | Ag2261, Run |
| Tissue Name | 150631675 | 152887692 | TissueName | 150631675 | 152887692 |
| Liver | 22.4 | 19.6 | Kidney (fetal) | 2.1 | 0.0 |
| adenocarcinoma | | | | | |
| Pancreas | 3.9 | 2.5 | Renal ca. 786-0 | 0.0 | 0.0 |
| Pancreatic ca. | 5.3 | 3.5 | Renal ca. A498 | 10.2 | 5.3 |
| CAPAN 2 | | | | | |
| Adrenal gland | 2.1 | 0.6 | Renal ca. RXF | 0.0 | 0.0 |
| | | | 393 | | |
| Thyroid | 7.0 | 9.8 | Renal ca. | 0.0 | 2.2 |
| | | | ACHN | | |
| Salivary gland | 1.9 | 2.1 | Renal ca. UO- | 0.0 | 0.0 |
| | | | 31 | | |
| Pituitary gland | 1.0 | 2.2 | Renal ca. TK- | 0.0 | 0.0 |
| | | | 10 | | |
| Brain (fetal) | 6.8 | 4.9 | Liver | 0.0 | 0.0 |
| Brain (whole) | 4.8 | 3.0 | Liver (fetal) | 7.6 | 0.0 |
| Brain (amygdala) | 4.6 | 5.3 | Liver ca. | 0.0 | 0.0 |
| | | | (hepatoblast) | | |
| | | | HepG2 | | |
| Brain (cerebellum) | 1.6 | 1.6 | Lung | 14.3 | 15.8 |
| Brain | 7.5 | 11.3 | Lung (fetal) | 15.1 | 15.4 |
| hippocampus | | | | | |
| Brain (substantia | 1.2 | 2.6 | Lung ca. (small | 1.6 | 0.0 |
| nigra | | | cell) LX-1 | | |
| Brain (thalamus) | 2.5 | 1.7 | Lung ca. (small | 29.5 | 19.1 |
| | | | cell) NCI-H69 | | |
| Cerebral Cortex | 0.0 | 0.0 | Lung ca. (s.cell | 11.0 | 5.1 |
| | | | var.) SHP-77 | | |
| Spinal cord | 1.7 | 2.1 | Lung ca. (large | 0.0 | 0.0 |
| | | | cell)NCI-H460 | | |
| glio/astro U87-MG | 0.0 | 0.0 | Lung ca. (non- | 0.0 | 1.2 |
| | | | sm. cell) A549 | | |
| glio/astro U-118- | 55.1 | 50.3 | Lung ca. (non- | 0.0 | 1.3 |
| MG | | | s.cell) NCI-H23 | | |
| astrocytoma | 0.0 | 7.5 | Lung ca. (non- | 0.0 | 1.7 |
| SW1783 | | | s.cell) HOP-62 | | |
| neuro*; met SK-N- | 0.0 | 0.0 | Lung ca. (non- | 8.0 | 8.3 |
| AS | | | s.cl) NCI-H522 | | |
| astrocytoma SF- | 1.9 | 4.7 | Lung ca. | 4.0 | 0.0 |
| 539 | | | (squam.) SW | | |
| | | | 900 | | |
| astrocytoma SNB- | 2.0 | 4.9 | Lung ca. | 15.8 | 10.2 |
| 75 | | | (squam.) NCI- | | |
| | | | H596 | | |
| glioma SNB-19 | 6.7 | 2.4 | Mammary | 7.2 | 4.1 |
| | | | gland | | |
| glioma U251 | 2.1 | 4.5 | Breast ca.* | 1.7 | 3.4 |
| | | | (pl.ef) MCF-7 | | |
| glioma SF-295 | 10.0 | 0.6 | Breast ca.* | 23.2 | 19.6 |
| | | | (pl.ef) MDA- | | |
| | | | MB-231 | | |
| Heart (Fetal) | 11.1 | 9.9 | Breast ca.* (pl. | 4.3 | 5.8 |
| | | | ef) T47D | | |
| Heart | 4.9 | 6.0 | Breast ca. BT- | 0.0 | 4.2 |
| | | | 549 | | |
| Skeletal muscle | 100.0 | 100.0 | Breast ca. | 0.0 | 0.0 |
| (Fetal) | | | MDA-N | | |
| Skeletal muscle | 5.5 | 8.4 | Ovary | 3.6 | 3.1 |
| Bone marrow | 0.0 | 0.0 | Ovarian ca. | 1.1 | 1.0 |
| | | | OVCAR-3 | | |
| Thymus | 10.0 | 3.9 | Ovarian ca. | 0.0 | 0.0 |
| | | | OVCAR-4 | | |
| Spleen | 3.8 | 4.2 | Ovarian ca. | 0.0 | 0.0 |
| | | | OVCAR-5 | | |
| Lymph node | 5.0 | 1.1 | Ovarian ca. | 1.3 | 4.3 |
| | | | OVCAR-8 | | |
| Colorectal | 3.4 | 5.4 | Ovarian ca. | 0.0 | 0.0 |
| | | | IGROV-1 | | |
| Stomach | 6.0 | 15.4 | Ovarian ca. | 7.5 | 16.0 |
| | | | (ascites) SK- | | |
| | | | OV-3 | | |
| Small intestine | 15.9 | 18.7 | Uterus | 17.8 | 15.1 |
| Colon ca. SW480 | 24.3 | 15.3 | Placenta | 4.6 | 8.2 |
| Colon ca.* SW620 | 0.0 | 0.0 | Prostate | 3.6 | 5.3 |
| (SW480 met) | | | | | |
| Colon ca. HT29 | 0.0 | 0.0 | Prostate ca.* | 1.7 | 1.5 |
| | | | (bone met) PC-3 | | |
| Colon ca. HCT- | 3.8 | 0.6 | Testis | 21.9 | 14.6 |
| 116 | | | | | |
| Colon ca. CaCo-2 | 0.0 | 0.8 | Melanoma | 3.1 | 4.7 |
| | | | Hs688(A).T | | |
| CC Well to Mod | 2.3 | 0.0 | Melanoma* | 0.4 | 1.3 |
| Diff (ODO3866) | | | (met) | | |
| | | | Hs688(B).T | | |
| Colon ca. HCC- | 0.0 | 0.0 | Melanoma | 0.0 | 0.0 |
| 2998 | | | UACC-62 | | |
| Gastric ca. (liver | 16.7 | 14.9 | Melanoma M14 | 0.0 | 0.0 |
| met) NCI-N87 | | | | | |
| Bladder | 1.6 | 3.2 | Melanoma LOX | 0.0 | 0.0 |
| | | | IMVI | | |
| Trachea | 24.3 | 33.7 | Melanoma* | 0.0 | 2.0 |
| | | | (met) SK-MEL-5 | | |
| Kidney | 0.0 | 0.0 | Adipose | 6.7 | 7.2 |

TABLE 62
Panel 2D

| | Rel. Exp. (%) | Rel. Exp. (%) | | Rel. Exp. (%) | Rel. Exp. (%) |
|---|---|---|---|---|---|
| | Ag2261, Run | Ag2261, Run | | Ag2261, Run | Ag2261, Run |
| Tissue Name | 150811744 | 152887693 | Tissue Name | 150811744 | 152887693 |
| Normal Colon | 19.1 | 19.8 | Kidney Margin | 2.4 | 0.0 |
| | | | 8120608 | | |
| CC Well to Mod | 0.0 | 5.8 | Kidney Cancer | 14.6 | 7.3 |
| Diff (ODO3866) | | | 8120613 | | |
| CC Margin | 19.5 | 12.5 | Kidney Margin | 4.8 | 1.5 |
| (ODO3866) | | | 8120614 | | |
| CC Gr.2 | 3.8 | 1.4 | Kidney Cancer | 0.0 | 0.0 |
| rectosigmoid | | | 9010320 | | |
| (ODO3868) | | | | | |
| CC Margin | 2.6 | 5.1 | Kidney Margin | 0.0 | 0.0 |
| (ODO3868) | | | 9010321 | | |
| CC Mod Diff | 6.0 | 2.9 | Normal Uterus | 9.7 | 2.8 |
| (ODO3920) | | | | | |
| CC Margin | 23.8 | 6.4 | Uterine Cancer | 85.9 | 41.5 |
| (ODO3920) | | | 064011 | | |
| CC Gr.2 ascend | 9.3 | 2.2 | Normal Thyroid | 15.2 | 7.3 |
| colon (ODO3921) | | | | | |
| CC Margin | 16.8 | 11.7 | Thyroid Cancer | 0.0 | 3.0 |
| (ODO3921) | | | | | |
| CC from Partial | 2.4 | 0.0 | Thyroid Cancer | 1.9 | 1.2 |
| Hepatectomy | | | A302152 | | |
| (ODO4309) Mets | | | | | |
| Liver Margin | 2.6 | 0.0 | Thyroid Margin | 2.6 | 2.8 |
| (ODO4309) | | | A302153 | | |
| Colon mets to lung | 7.9 | 4.5 | Normal Breast | 16.2 | 2.7 |
| (OD04451-01) | | | | | |
| Lung Margin | 11.3 | 12.9 | Breast Cancer | 78.5 | 29.7 |
| (OD04451-02) | | | | | |
| Normal Prostate | 6.3 | 2.6 | Breast Cancer | 37.6 | 23.8 |
| 6546-1 | | | (OD04590-01) | | |
| Prostate Cancer | 17.8 | 7.3 | Breast Cancer | 100.0 | 24.5 |
| (OD04410) | | | Mets | | |
| | | | (OD04590-03) | | |
| Prostate Margin | 10.7 | 7.4 | Breast Cancer | 94.0 | 45.4 |
| (OD04410) | | | Metastasis | | |
| Prostate Cancer | 4.7 | 4.4 | Breast Cancer | 25.7 | 24.8 |
| (OD04720-01) | | | | | |
| Prostate Margin | 13.9 | 5.6 | Breast Cancer | 23.2 | 7.1 |
| (OD04720-02) | | | | | |
| Normal Lung | 36.6 | 14.3 | Breast Cancer | 33.0 | 7.5 |
| | | | 9100266 | | |
| Lung Met to | 1.0 | 0.0 | Breast Margin | 7.6 | 7.6 |
| Muscle | | | 9100265 | | |
| (ODO4286) | | | | | |
| Muscle Margin | 31.0 | 38.2 | Breast Cancer | 13.9 | 0.9 |
| (ODO4286) | | | A209073 | | |
| Lung Malignant | 81.8 | 100.0 | Breast Margin | 2.5 | 0.0 |
| Cancer | | | A2090734 | | |
| (OD03126) | | | | | |
| Lung Margin | 35.8 | 18.2 | Normal Liver | 0.0 | 0.0 |
| (OD03126) | | | | | |
| Lung Cancer | 57.0 | 39.5 | Liver Cancer | 0.0 | 0.0 |
| (OD04404) | | | | | |
| Lung Margin | 9.4 | 11.8 | Liver Cancer | 4.8 | 1.7 |
| (OD04404) | | | 1025 | | |
| Lung Cancer | 37.1 | 42.0 | Liver Cancer | 7.1 | 0.0 |
| (OD04565) | | | 1026 | | |
| Lung Margin | 22.7 | 9.3 | Liver Cancer | 4.8 | 0.0 |
| (OD04565) | | | 6004-T | | |
| Lung Cancer | 5.3 | 6.4 | Liver Tissue | 4.4 | 1.8 |
| (OD04237-01) | | | 6004-N | | |
| Lung Margin | 78.5 | 32.8 | Liver Cancer | 0.0 | 6.0 |
| (OD04237-02) | | | 6005-T | | |
| Ocular Mel Met to | 0.0 | 0.0 | Liver Tissue | 0.0 | 1.8 |
| Liver (ODO4310) | | | 6005-N | | |
| Liver Margin | 2.4 | 0.0 | Normal Bladder | 2.4 | 3.0 |
| (ODO4310) | | | | | |
| Melanoma | 13.0 | 0.0 | Bladder Cancer | 8.5 | 4.9 |
| Metastasis | | | | | |
| Lung Margin | 96.6 | 50.0 | Bladder Cancer | 17.0 | 11.8 |
| (OD04321) | | | | | |
| Normal Kidney | 0.0 | 0.0 | Bladder Cancer | 10.0 | 5.7 |
| | | | (OD04718-01) | | |
| Kidney Ca, | 0.0 | 0.0 | Bladder Normal | 19.3 | 27.5 |
| Nuclear grade 2 | | | Adjacent | | |
| (OD04338) | | | (OD04718-03) | | |
| Kidney Margin | 4.0 | 4.6 | Normal Ovary | 13.6 | 12.4 |
| (OD04338) | | | | | |
| Kidney Ca | 0.0 | 3.3 | Ovarian Cancer | 37.9 | 2.1 |
| Nuclear grade 1/2 | | | | | |
| (OD04339) | | | | | |
| Kidney Margin | 18.7 | 0.0 | Ovarian Cancer | 18.4 | 3.7 |
| (OD04339) | | | (OD04768-07) | | |
| Kidney Ca, Clear | 8.8 | 11.7 | Ovary Margin | 28.3 | 12.2 |
| cell type | | | (OD04768-08) | | |
| (OD04340) | | | | | |
| Kidney Margin | 0.0 | 2.0 | Normal | 48.3 | 17.3 |
| (OD04340) | | | Stomach | | |
| Kidney Ca, | 3.5 | 4.0 | Gastric Cancer | 0.0 | 0.0 |
| Nuclear grade 3 | | | 9060358 | | |
| (OD04348) | | | | | |
| Kidney Margin | 2.0 | 1.7 | Stomach | 9.9 | 3.0 |
| (OD04348) | | | Margin | | |
| | | | 9060359 | | |
| Kidney Cancer | 9.3 | 0.0 | Gastric Cancer | 20.7 | 10.4 |
| (OD04622-01) | | | 9060395 | | |
| Kidney Margin | 0.0 | 6.3 | Stomach | 10.0 | 12.2 |
| (OD04622-03) | | | Margin | | |
| | | | 9060394 | | |
| Kidney Cancer | 0.0 | 0.0 | Gastric Cancer | 8.7 | 1.5 |
| (OD04450-01) | | | 9060397 | | |
| Kidney Margin | 0.0 | 0.0 | Stomach | 7.5 | 6.2 |
| (OD04450-03) | | | Margin | | |
| | | | 9060396 | | |
| Kidney Cancer | 0.0 | 0.7 | Gastric Cancer | 10.7 | 4.8 |
| 8120607 | | | 064005 | | |

TABLE 63
Panel 4D

| | Rel. Exp. (%) | | Rel. Exp. (%) |
|---|---|---|---|
| | Ag2261, Run | | Ag2261, Run |
| Tissue Name | 152887762 | Tissue Name | 152887762 |
| Secondary Th1 act | 0.0 | HUVEC IL-1beta | 0.0 |
| Secondary Th2 act | 0.0 | HUVEC IFN gamma | 3.7 |
| Secondary Tr1 act | 0.0 | HUVEC TNF alpha + IFN | 0.0 |
| | | gamma | |
| Secondary Th1 rest | 0.0 | HUVEC TNF alpha + IL4 | 4.3 |
| Secondary Th2 rest | 0.0 | HUVEC IL-11 | 4.0 |
| Secondary Tr1 rest | 0.0 | Lung Microvascular EC none | 7.2 |
| Primary Th1 act | 0.0 | Lung Microvascular EC | 0.0 |
| | | TNF alpha + IL-1beta | |
| Primary Th2 act | 0.0 | Microvascular Dermal EC | 8.4 |
| | | none | |
| Primary Tr1 act | 0.0 | Microsvasular Dermal EC | 0.0 |
| | | TNF alpha + IL-1beta | |
| Primary Th1 rest | 0.0 | Bronchial epithelium | 0.0 |
| | | TNF alpha + IL1beta | |
| Primary Th2 rest | 0.0 | Small airway epithelium | 5.9 |
| | | none | |
| Primary Tr1 rest | 0.0 | Small airway epithelium | 24.3 |
| | | TNF alpha + IL-1beta | |
| CD45RA CD4 | 0.0 | Coronery artery SMC rest | 0.0 |
| lymphocyte act | | | |
| CD45RO CD4 | 0.0 | Coronery artery SMC | 0.0 |
| lymphocyte act | | TNF alpha + IL-1beta | |
| CD8 lymphocyte act | 0.0 | Astrocytes rest | 3.3 |
| Secondary CD8 | 0.0 | Astrocytes TNF alpha + IL- | 0.0 |
| lymphocyte rest | | 1beta | |
| Secondary CD8 | 1.6 | KU-812 (Basophil) rest | 0.0 |
| lymphocyte act | | | |
| CD4 lymphocyte none | 0.0 | KU-812 (Basophil) | 0.0 |
| | | PMA/ionomycin | |
| 2ry Th1/Th2/Tr1_anti- | 0.0 | CCD1106 (Keratinocytes) | 47.3 |
| CD95 CH11 | | none | |
| LAK cells rest | 3.5 | CCD1106 (Keratinocytes) | 9.0 |
| | | TNF alpha + IL-1beta | |
| LAK cells IL-2 | 0.0 | Liver cirrhosis | 32.8 |
| LAK cells IL-2 + IL-12 | 0.0 | Lupus kidney | 0.0 |
| LAK cells IL-2 + IFN | 0.0 | NCI-H292 none | 3.8 |
| gamma | | | |
| LAK cells IL-2 + IL-18 | 0.0 | NCI-H292 IL-4 | 8.0 |
| LAK cells | 26.1 | NCI-H292 IL-9 | 0.0 |
| PMA/ionomycin | | | |
| NK Cells IL-2 rest | 0.0 | NCI-H292 IL-13 | 13.8 |
| Two Way MLR 3 day | 0.0 | NCI-H292 IFN gamma | 16.2 |
| Two Way MLR 5 day | 0.0 | HPAEC none | 6.7 |
| Two Way MLR 7 day | 0.0 | HPAEC TNF alpha + IL-1 | 0.0 |
| | | beta | |
| PBMC rest | 0.0 | Lung fibroblast none | 7.6 |
| PBMC PWM | 0.0 | Lung fibroblast TNF alpha + | 3.1 |
| | | IL-1beta | |
| PBMC PHA-L | 0.0 | Lung fibroblast IL-4 | 4.3 |
| Ramos (B cell) none | 0.0 | Lung fibroblast IL-9 | 12.7 |
| Ramos (B cell) | 0.0 | Lung fibroblast IL-13 | 6.8 |
| ionomycin | | | |
| B lymphocytes PWM | 0.0 | Lung fibroblast IFN gamma | 30.4 |
| B lymphocytes CD40L | 3.1 | Dermal fibroblast CCD1070 | 0.0 |
| and IL-4 | | rest | |
| EOL-1 dbcAMP | 0.0 | Dermal fibroblast CCD1070 | 5.2 |
| | | TNF alpha | |
| EOL-1 dbcAMP | 3.5 | Dermal fibroblast CCD1070 | 0.0 |
| PMA/ionomycin | | IL-1beta | |
| Dendritic cells none | 0.0 | Dermal fibroblast IFN | 28.5 |
| | | gamma | |
| Dendritic cells LPS | 0.0 | Dermal fibroblast IL-4 | 42.9 |
| Dendritic cells anti- | 0.0 | IBD Colitis 2 | 2.2 |
| CD40 | | | |
| Monocytes rest | 0.0 | IBD Crohn's | 3.1 |
| Monocytes LPS | 0.0 | Colon | 100.0 |
| Macrophages rest | 0.0 | Lung | 36.3 |
| Macrophages LPS | 0.0 | Thymus | 0.0 |
| HUVEC none | 0.0 | Kidney | 4.0 |
| HUVEC starved | 17.4 | | |

Panel 1.3D Summary: Ag2261 The 88091010_EXT gene is expressed at moderate levels in a number of metabolic tissues, with highest overall expression seen in fetal skeletal muscle (CTs=30.4-31.8). The higher levels of expression in fetal skeletal muscle when compared to adult skeletal muscle suggest that the protein product encoded by the 88091010_EXT gene may be useful in treating muscular dystrophy, Lesch-Nyhan syndrome, myasthenia gravis and other conditions that result in weak or dystrophic muscle. This gene is also expressed in adipose, thyroid and heart. Since biologic cross-talk between adipose and thyroid is a component of some forms of obesity, this gene product may be a protein therapeutic for the treatment of metabolic disease, including obesity and Type 2 diabetes.

Panel 2D Summary: Ag2261 The expression of this gene was assessed in two independent runs on panel 2D. This gene is consistently expressed in samples of breast cancer, uterine cancer and lung cancer when compared to their respective normal adjacent tissue controls. Thus, the expression of this gene could be used to distinguish breast cancer, lung cancer or uterine cancer from their normal tissues. Moreover, therapeutic modulation of this gene, through the use of small molecule drugs, antibodies or protein therapeutics might be of use in the treatment of breast, lung or uterine cancer.

Panel 4D Summary: Ag 2261: This transcript is expressed at a low, but significant level in colon (CT 33.5). Low levels of expression of this transcript are also found in the lung, keratinocytes and dermal fibroblast. Thus, this transcript could be used as a marker for thymic, lung and skin tissues. The putative Wnt-14 encoded by this transcript may play an important role in the normal homeostasis of these tissues. Therefore, therapeutics designed with the protein encoded for by this transcript could be important for maintaining or restoring normal function to these organs during inflammation.

NOV9

Expression of NOV9 was assessed using the primer-probe set Ag2303, described in Table 64. Results of the RTQ-PCR runs are shown in Tables 65 and 66.

TABLE 64

Probe Name Ag2303

| | | | | |
|---|---|---|---|---|
| | | | | SEQ |
| | | | Start | ID |
| Primers | Sequences | Length | Position | NO: |

| | | | | |
|---|---|---|---|---|
| Forward | 5′-CATTGAGAGCGATAAGTTCA- | 22 | 510 | 181 |
| | CA-3′ | | | |
| Probe | TET-5′-AGAATGTGGAGCTCAA- | 26 | 548 | 182 |
| | CATCCACCTG-3′-TAMRA | | | |
| Reverse | 5′-GATGCACGCTGAAGTCATTC- | 20 | 579 | 183 |
| | 3′ | | | |

TABLE 65
Panel 1.3D

| | Rel. Exp. (%) | | Rel. Exp. (%) |
|---|---|---|---|
| | Ag2303, Run | | Ag2303, Run |
| Tissue Name | 167985232 | Tissue Name | 167985232 |
| Liver adenocarcinoma | 19.1 | Kidney (fetal) | 25.5 |
| Pancreas | 5.1 | Renal ca. 786-0 | 7.4 |
| Pancreatic ca. CAPAN 2 | 20.0 | Renal ca. A498 | 6.8 |
| Adrenal gland | 2.7 | Renal ca. RXF 393 | 15.5 |
| Thyroid | 2.3 | Renal ca. ACHN | 3.9 |
| Salivary gland | 7.2 | Renal ca. UO-31 | 6.3 |
| Pituitary gland | 5.0 | Renal ca. TK-10 | 16.4 |
| Brain (fetal) | 31.9 | Liver | 6.1 |
| Brain (whole) | 58.2 | Liver (fetal) | 6.7 |
| Brain (amygdala) | 33.9 | Liver ca. (hepatoblast) HepG2 | 11.7 |
| Brain (cerebellum) | 55.5 | Lung | 14.7 |
| Brain (hippocampus) | 23.3 | Lung (fetal) | 11.0 |
| Brain (substantia nigra) | 15.3 | Lung ca. (small cell) LX-1 | 36.6 |
| Brain (thalamus) | 21.9 | Lung ca. (small cell) NCI- | 15.0 |
| | | H69 | |
| Cerebral Cortex | 80.1 | Lung ca. (s.cell var.) SHP-77 | 60.7 |
| Spinal cord | 8.4 | Lung ca. (large cell)NCI- | 5.4 |
| | | H460 | |
| glio/astro U87-MG | 12.0 | Lung ca. (non-sm. cell) A549 | 14.3 |
| glio/astro U-118-MG | 10.8 | Lung ca. (non-s.cell) NCI- | 37.4 |
| | | H23 | |
| astrocytoma SW1783 | 15.5 | Lung ca. (non-s.cell) HOP-62 | 14.5 |
| neuro*; met SK-N-AS | 7.0 | Lung ca. (non-s.cl) NCI-H522 | 15.6 |
| astrocytoma SF-539 | 9.9 | Lung ca. (squam.) SW 900 | 16.2 |
| astrocytoma SNB-75 | 15.9 | Lung ca. (squam.) NCI-H596 | 33.2 |
| glioma SNB-19 | 8.7 | Mammary gland | 17.6 |
| glioma U251 | 20.7 | Breast ca.* (pl.ef) MCF-7 | 17.1 |
| glioma SF-295 | 7.9 | Breast ca.* (pl.ef) MDA-MB- | 6.7 |
| | | 231 | |
| Heart (Fetal) | 46.0 | Breast ca.* (pl. ef) T47D | 29.7 |
| Heart | 9.8 | Breast ca. BT-549 | 4.0 |
| Skeletal muscle (Fetal) | 30.6 | Breast ca. MDA-N | 10.4 |
| Skeletal muscle | 26.6 | Ovary | 7.9 |
| Bone marrow | 29.5 | Ovarian ca. OVCAR-3 | 13.3 |
| Thymus | 32.3 | Ovarian ca. OVCAR-4 | 14.3 |
| Spleen | 26.4 | Ovarian ca. OVCAR-5 | 62.4 |
| Lymph node | 26.2 | Ovarian ca. OVCAR-8 | 3.9 |
| Colorectal | 11.0 | Ovarian ca. IGROV- 1 | 6.2 |
| Stomach | 7.9 | Ovarian ca. (ascites) SK-OV-3 | 47.0 |
| Small intestine | 5.6 | Uterus | 5.0 |
| Colon ca. SW480 | 15.6 | Placenta | 3.2 |
| Colon ca.* SW620 (SW480 | 100.0 | Prostate | 8.0 |
| met) | | | |
| Colon ca. HT29 | 19.5 | Prostate ca.* (bone met) PC-3 | 21.5 |
| Colon ca. HCT-116 | 16.6 | Testis | 5.0 |
| Colon ca. CaCo-2 | 21.9 | Melanoma Hs688(A).T | 4.3 |
| CC Well to Mod Diff | 13.1 | Melanoma* (met) | 3.6 |
| (ODO3866) | | Hs688(B).T | |
| Colon ca. HCC-2998 | 33.9 | Melanoma UACC-62 | 7.0 |
| Gastric ca. (liver met) NCI-N87 | 18.8 | Melanoma M14 | 5.0 |
| Bladder | 7.2 | Melanoma LOX IMVI | 13.3 |
| Trachea | 4.0 | Melanoma* (met) SK-MEL-5 | 7.8 |
| Kidney | 7.6 | Adipose | 13.8 |

TABLE 66
Panel 4D

| | Rel. Exp. (%) | | Rel. Exp. (%) |
|---|---|---|---|
| | Ag2303, Run | | Ag2303, Run |
| Tissue Name | 151630338 | Tissue Name | 151630338 |
| Secondary Th1 act | 69.7 | HUVEC IL-1beta | 2.8 |
| Secondary Th2 act | 51.4 | HUVEC IFN gamma | 15.7 |
| Secondary Tr1 act | 66.0 | HUVEC TNF alpha + IFN | 7.2 |
| | | gamma | |
| Secondary Th1 rest | 24.5 | HUVEC TNF alpha + IL4 | 7.2 |
| Secondary Th2 rest | 28.9 | HUVEC IL-11 | 5.9 |
| Secondary Tr1 rest | 29.1 | Lung Microvascular EC none | 6.8 |
| Primary Th1 act | 53.2 | Lung Microvascular EC | 5.4 |
| | | TNF alpha + IL-1beta | |
| Primary Th2 act | 44.4 | Microvascular Dermal EC | 10.1 |
| | | none | |
| Primary Tr1 act | 66.0 | Microsvasular Dermal EC | 6.7 |
| | | TNF alpha + IL-1beta | |
| Primary Th1 rest | 89.5 | Bronchial epithelium | 7.2 |
| | | TNF alpha + IL1beta | |
| Primary Th2 rest | 66.0 | Small airway epithelium none | 4.1 |
| Primary Tr1 rest | 46.7 | small airway epithelium | 20.4 |
| | | TNF alpha + IL-1beta | |
| CD45RA CD4 lymphocyte act | 36.3 | Coronery artery SMC rest | 7.7 |
| CD45RO CD4 lymphocyte act | 55.5 | Coronery artery SMC | 6.1 |
| | | TNF alpha + IL-1beta | |
| CD8 lymphocyte act | 56.3 | Astrocytes rest | 4.4 |
| Secondary CD8 lymphocyte rest | 47.6 | Astrocytes TNF alpha + IL- | 3.0 |
| | | 1beta | |
| Secondary CD8 lymphocyte act | 48.0 | KU-812 (Basophil) rest | 17.3 |
| CD4 lymphocyte none | 15.2 | KU-812 (Basophil) | 31.2 |
| | | PMA/ionomycin | |
| 2ry Th1/Th2/Tr1_anti-CD95 | 41.2 | CCD1106 (Keratinocytes) | 11.8 |
| CH11 | | none | |
| LAK cells rest | 34.4 | CCD1106 (Keratinocytes) | 9.9 |
| | | TNF alpha + IL-1beta | |
| LAK cells IL-2 | 69.3 | Liver cirrhosis | 2.0 |
| LAK cells IL-2 + IL-12 | 55.9 | Lupus kidney | 2.1 |
| LAK cells IL-2 + IFN gamma | 63.3 | NCI-H292 none | 21.0 |
| LAK cells IL-2 + IL-18 | 57.0 | NCI-H292 IL-4 | 33.2 |
| LAK cells PMA/ionomycin | 9.6 | NCI-H292 IL-9 | 33.2 |
| NK Cells IL-2 rest | 47.6 | NCI-H292 IL-13 | 20.9 |
| Two Way MLR 3 day | 38.7 | NCI-H292 IFN gamma | 25.0 |
| Two Way MLR 5 day | 39.5 | HPAEC none | 8.2 |
| Two Way MLR 7 day | 42.0 | HPAEC TNF alpha + IL-1 | 8.6 |
| | | beta | |
| PBMC rest | 21.5 | Lung fibroblast none | 5.9 |
| PBMC PWM | 100.0 | Lung fibroblast TNF alpha + | 6.4 |
| | | IL-1beta | |
| PBMC PHA-L | 73.7 | Lung fibroblast IL-4 | 12.2 |
| Ramos (B cell) none | 54.3 | Lung fibroblast IL-9 | 9.9 |
| Ramos (B cell) ionomycin | 78.5 | Lung fibroblast IL-13 | 9.6 |
| B lymphocytes PWM | 90.1 | Lung fibroblast IFN gamma | 11.6 |
| B lymphocytes CD40L and IL-4 | 53.6 | Dermal fibroblast CCD1070 | 12.5 |
| | | rest | |
| EOL-1 dbcAMP | 57.4 | Dermal fibroblast CCD1070 | 67.8 |
| | | TNF alpha | |
| EOL-1 dbcAMP | 18.8 | Dermal fibroblast CCD1070 | 9.7 |
| PMA/ionomycin | | IL-1beta | |
| Dendritic cells none | 22.1 | Dermal fibroblast IFN gamma | 5.5 |
| Dendritic cells LPS | 15.9 | Dermal fibroblast IL-4 | 7.4 |
| Dendritic cells anti-CD40 | 22.2 | IBD Colitis 2 | 2.0 |
| Monocytes rest | 45.4 | IBD Crohn's | 1.4 |
| Monocytes LPS | 17.3 | Colon | 20.4 |
| Macrophages rest | 36.1 | Lung | 14.0 |
| Macrophages LPS | 18.0 | Thymus | 10.6 |
| HUVEC none | 13.7 | Kidney | 31.6 |
| HUVEC starved | 19.8 | | |

Panel 1.3D Summary: Ag2303

NOV9 is widely expressed across the panel, with highest expression in a colon cancer cell line SW620 (CT=26.4). Of note is the difference in expression between the related colon cancer cell lines SW620 and SW480. SW480 represents the primary lesion from a patient with colon cancer, while SW620 represents a metastasis from the same patient. The difference in expression of this gene between the SW620 and SW480 cell lines indicates that it could be used to distingush these cells, or others like them. Moreover, therapeutic modulation of NOV9, through the use of small molecule drugs, antibodies or protein therapeutics, may be effective in the treatment of metastatic colon cancer.

Among tissues with metabolic function, NOV9 is moderately expressed in the pancreas, adrenal, thyroid, pituitary, adipose, adult and fetal heart, and adult and fetal liver. This expression profile suggests that the NOV9 product may be an important small molecule target for the treatment of metabolic disease in any or all of these tissues, including obesity and diabetes.

NOV9, which encodes a beta-adrenergic receptor kinase, also shows high expression in all regions of the brain examined, especially in the cerebral cortex (CT=26.7) The beta adrenergic receptors have been shown to play a role in memory formation and in clinical depression. Since many current anti-depressants produce undesired side effects as a result of non-specific binding (to other receptors), this gene is therefore an excellent small molecule target for the treatment of clinical depression without side effects. Furthermore, the role of beta adrenergic receptors in memory consolidation suggests that the NOV9 gene product would also be useful as a small molecule target for the treatment of Alzheimer's disease, vascular dementia, or any memory loss disorder.

References:

1. Feighner J P. Mechanism of action of antidepressant medications. J Clin Psychiatry 1999;60 Suppl 4:4-11; discussion 12-3

The psychopharmacology of depression is a field that has evolved rapidly in just under 5 decades. Early antidepressant medications—tricyclic antidepressants (TCAs) and monoamine oxidase inhibitors (MAOIs)—were discovered through astute clinical observations. These first-generation medications were effective because they enhanced serotonergic or noradrenergic mechanisms or both. Unfortunately, the TCAs also blocked histaminic, cholinergic, and alpha I -adrenergic receptor sites, and this action brought about unwanted side effects such as weight gain, dry mouth, constipation, drowsiness, and dizziness. MAOIs can interact with tyramine to cause potentially lethal hypertension and present potentially dangerous interactions with a number of medications and over-the-counter drugs. The newest generation of antidepressants, including the single-receptor selective serotonin reuptake inhibitors (SSRIs) and multiple-receptor antidepressants venlafaxine, mirtazapine, bupropion, trazodone, and nefazodone, target one or more specific brain receptor sites without, in most cases, activating unwanted sites such as histamine and acetylcholine. This paper discusses the new antidepressants, particularly with regard to mechanism of action, and looks at future developments in the treatment of depression.

2. Ferry B, McGaugh J L. Role of amygdala norepinephrine in mediating stress hormone regulation of memory storage. Acta Pharmacol Sin Jun. 21, 2000;(6):481-93

There is extensive evidence indicating that the noradrenergic system of the amygdala, particularly the basolateral nucleus of the amygdala (BLA), is involved in memory consolidation. This article reviews the central hypothesis that stress hormones released during emotionally arousing experiences activate noradrenergic mechanisms in the BLA, resulting in enhanced memory for those events. Findings from experiments using rats have shown that the memory-modulatory effects of the adrenocortical stress hormones epinephrine and glucocorticoids involve activation of beta-adrenoceptors in the BLA. In addition, both behavioral and microdialysis studies have shown that the noradrenergic system of the BLA also mediates the influences of other neuromodulatory systems such as opioid peptidergic and GABAergic systems on memory storage. Other findings indicate that this stress hormone-induced activation of noradrenergic mechanisms in the BLA regulates memory storage in other brain regions.

Panel 4D Summary: Ag2303

NOV9, a beta-adrenergic receptor kinase homolog, is highly expressed (CTs 26-29) in a wide range of cells that play a significance role in the immune response. Highest expression of this gene is found in activated B and T cells. Therefore, inhibition of the function of the protein encoded by NOV9 with a small molecule drug may block the functions of B cells or T cells and could be beneficial in the treatment of patients suffering from autoimmune and inflammatory diseases such as asthma, allergies, inflammatory bowel disease, lupus erythematosus, or rheumatoid arthritis.

NOV10

Expression of NOV10 was assessed using the primer-probe set Ag231 1, described in Table 67. Results of the RTQ-PCR runs are shown in Tables 68, 69, 70 and 71.

TABLE 67

Probe Name Ag2311

| | | | | |
|---|---|---|---|---|
| | | | | SEQ |
| | | | Start | ID |
| Primers | Sequences | Length | Position | NO: |

| | | | | |
|---|---|---|---|---|
| Forward | 5′-CTCTGGGGACTCCTAATTTC- | 22 | 2913 | 184 |
| | TG-3′ | | | |
| Probe | TET-5′-CCCAGCCTAAAGCAGG- | 26 | 2939 | 185 |
| | GATCAGTCTT-3′-TAMRA | | | |
| Reverse | 5′-TCCAAGGATTTATTCCACAA- | 22 | 2966 | 186 |
| | GA-3′ | | | |

TABLE 68
CNS_neurodegeneration_v1.0

| | Rel. Exp. (%) | | Rel. Exp. (%) |
|---|---|---|---|
| | Ag2311, Run | | Ag2311, Run |
| Tissue Name | 208253895 | Tissue Name | 208253895 |
| AD 1 Hippo | 33.4 | Control (Path) 3 Temporal Ctx | 13.4 |
| AD 2 Hippo | 46.3 | Control (Path) 4 Temporal Ctx | 44.8 |
| AD 3 Hippo | 12.9 | AD 1 Occipital Ctx | 36.1 |
| AD 4 Hippo | 15.4 | AD 2 Occipital Ctx (Missing) | 0.0 |
| AD 5 Hippo | 87.7 | AD 3 Occipital Ctx | 10.5 |
| AD 6 Hippo | 41.2 | AD 4 Occipital Ctx | 23.2 |
| Control 2 Hippo | 34.4 | AD 5 Occipital Ctx | 40.1 |
| Control 4 Hippo | 29.7 | AD 5 Occipital Ctx | 28.3 |
| Control (Path) 3 Hippo | 13.0 | Control 1 Occipital Ctx | 8.8 |
| AD 1 Temporal Ctx | 39.2 | Control 2 Occipital Ctx | 57.4 |
| AD 2 Temporal Ctx | 46.7 | Control 3 Occipital Ctx | 32.3 |
| AD 3 Temporal Ctx | 12.2 | Control 4 Occipital Ctx | 13.6 |
| AD 4 Temporal Ctx | 42.9 | Control (Path) 1 Occipital Ctx | 67.4 |
| AD 5 Inf Temporal Ctx | 100.0 | Control (Path) 2 Occipital Ctx | 26.8 |
| AD 5 Sup Temporal Ctx | 57.8 | Control (Path) 3 Occipital Ctx | 12.5 |
| AD 6 Inf Temporal Ctx | 48.3 | Control (Path) 4 Occipital Ctx | 36.6 |
| AD 6 Sup Temporal Ctx | 42.6 | Control 1 Parietal Ctx | 14.1 |
| Control 1 Temporal Ctx | 15.7 | Control 2 Parietal Ctx | 71.7 |
| Control 2 Temporal Ctx | 37.4 | Control 3 Parietal Ctx | 29.1 |
| Control 3 Temporal Ctx | 25.5 | Control (Path) 1 Parietal Ctx | 39.5 |
| Control 3 Temporal Ctx | 23.5 | Control (Path) 2 Parietal Ctx | 31.2 |
| Control (Path) 1 Temporal Ctx | 59.5 | Control (Path) 3 Parietal Ctx | 11.6 |
| Control (Path) 2 Temporal Ctx | 35.8 | Control (Path) 4 Parietal Ctx | 58.2 |

TABLE 69
Panel 1.3D

| | Rel. Exp. (%) | | Rel. Exp. (%) |
|---|---|---|---|
| | Ag2311, Run | | Ag2311, Run |
| Tissue Name | 165627680 | Tissue Name | 165627680 |
| Liver adenocarcinoma | 1.7 | Kidney (fetal) | 6.7 |
| Pancreas | 10.5 | Renal ca. 786-0 | 1.3 |
| Pancreatic ca. CAPAN 2 | 5.4 | Renal ca. A498 | 6.5 |
| Adrenal gland | 22.2 | Renal ca. RXF 393 | 3.3 |
| Thyroid | 21.5 | Renal ca. ACHN | 0.9 |
| Salivary gland | 10.6 | Renal ca. UO-31 | 1.1 |
| Pituitary gland | 24.7 | Renal ca. TK-10 | 1.3 |
| Brain (fetal) | 15.0 | Liver | 7.5 |
| Brain (whole) | 23.7 | Liver (fetal) | 10.8 |
| Brain (amygdala) | 24.7 | Liver ca. (hepatoblast) HepG2 | 2.0 |
| Brain (cerebellum) | 24.5 | Lung | 27.5 |
| Brain (hippocampus) | 35.1 | Lung (fetal) | 7.5 |
| Brain (substantia nigra) | 100.0 | Lung ca. (small cell) LX-1 | 4.6 |
| Brain (thalamus) | 27.7 | Lung ca. (small cell) NCI- | 0.0 |
| | | H69 | |
| Cerebral Cortex | 9.9 | Lung ca. (s.cell var.) SHP-77 | 5.4 |
| Spinal cord | 32.5 | Lung ca. (large cell) NCI- | 19.1 |
| | | H460 | |
| glio/astro U87-MG | 4.2 | Lung ca. (non-sm. cell) A549 | 3.2 |
| glio/astro U-118-MG | 9.2 | Lung ca. (non-s.cell) NCI- | 5.1 |
| | | H23 | |
| astrocytoma SW1783 | 3.7 | Lung ca. (non-s.cell) HOP-62 | 3.5 |
| neuro*; met SK-N-AS | 12.2 | Lung ca. (non-s.cl) NCI-H522 | 1.4 |
| astrocytoma SF-539 | 4.1 | Lung ca. (squam.) SW 900 | 1.9 |
| astrocytoma SNB-75 | 3.7 | Lung ca. (squam.) NCI-H596 | 0.3 |
| glioma SNB-19 | 5.8 | Mammary gland | 18.0 |
| glioma U251 | 28.9 | Breast ca.* (pl.ef) MCF-7 | 6.1 |
| glioma SF-295 | 4.5 | Breast ca.* (pl.ef) MDA-MB- | 3.8 |
| | | 231 | |
| Heart (Fetal) | 5.4 | Breast ca.* (pl.ef) T47D | 6.3 |
| Heart | 12.3 | Breast ca. BT-549 | 1.0 |
| Skeletal muscle (Fetal) | 4.6 | Breast ca. MDA-N | 2.1 |
| Skeletal muscle | 22.8 | Ovary | 2.0 |
| Bone marrow | 15.1 | Ovarian ca. OVCAR-3 | 4.3 |
| Thymus | 17.9 | Ovarian ca. OVCAR-4 | 1.8 |
| Spleen | 21.9 | Ovarian ca. OVCAR-5 | 9.8 |
| Lymph node | 27.7 | Ovarian ca. OVCAR-8 | 1.0 |
| Colorectal | 3.0 | Ovarian ca. IGROV-1 | 0.8 |
| Stomach | 12.9 | Ovarian ca. (ascites) SK-OV-3 | 2.8 |
| Small intestine | 66.4 | Uterus | 29.3 |
| Colon ca. SW480 | 2.4 | Placenta | 8.2 |
| Colon ca.* SW620 (SW480 | 4.1 | Prostate | 28.7 |
| met) | | | |
| Colon ca. HT29 | 2.4 | Prostate ca.* (bone met) PC-3 | 1.7 |
| Colon ca. HCT-116 | 2.7 | Testis | 38.7 |
| Colon ca. CaCo-2 | 3.0 | Melanoma Hs688(A).T | 1.3 |
| CC Well to Mod Diff | 5.8 | Melanoma* (met) | 2.0 |
| (ODO3866) | | Hs688(B).T | |
| Colon ca. HCC-2998 | 3.5 | Melanoma UACC-62 | 2.9 |
| Gastric ca. (liver met) NCI-N87 | 13.8 | Melanoma M14 | 11.0 |
| Bladder | 4.5 | Melanoma LOX IMVI | 0.2 |
| Trachea | 13.1 | Melanoma* (met) SK-MEL-5 | 2.9 |
| Kidney | 14.7 | Adipose | 9.7 |

TABLE 70
Panel 2.2

| | Rel. Exp. (%) | | Rel. Exp. (%) |
|---|---|---|---|
| | Ag2311, Run | | Ag2311, Run |
| Tissue Name | 174370590 | Tissue Name | 174370590 |
| Normal Colon | 9.4 | Kidney Margin (OD04348) | 100.0 |
| Colon cancer (OD06064) | 1.2 | Kidney malignant cancer | 9.3 |
| | | (OD06204B) | |
| Colon Margin (OD06064) | 0.6 | Kidney normal adjacent tissue | 17.6 |
| | | (OD06204E) | |
| Colon cancer (OD06159) | 1.5 | Kidney Cancer (OD04450-01) | 41.2 |
| Colon Margin (OD06159) | 5.5 | Kidney Margin (OD04450-03) | 14.9 |
| Colon cancer (OD06297-04) | 1.1 | Kidney Cancer 8120613 | 2.9 |
| Colon Margin (OD06297-015) | 9.6 | Kidney Margin 8120614 | 9.8 |
| CC Gr.2 ascend colon | 9.8 | Kidney Cancer 9010320 | 6.7 |
| (ODO3921) | | | |
| CC Margin (ODO3921) | 3.6 | Kidney Margin 9010321 | 5.4 |
| Colon cancer metastasis | 5.5 | Kidney Cancer 8120607 | 6.3 |
| (OD06104) | | | |
| Lung Margin (OD06104) | 1.2 | Kidney Margin 8120608 | 6.3 |
| Colon mets to lung (OD04451- | 22.2 | Normal Uterus | 17.6 |
| 01) | | | |
| Lung Margin (OD04451-02) | 12.0 | Uterine Cancer 064011 | 11.2 |
| Normal Prostate | 6.2 | Normal Thyroid | 3.3 |
| Prostate Cancer (OD04410) | 4.3 | Thyroid Cancer | 11.1 |
| Prostate Margin (OD04410) | 9.6 | Thyroid Cancer A302152 | 33.4 |
| Normal Ovary | 17.3 | Thyroid Margin A302153 | 9.7 |
| Ovarian cancer (OD06283-03) | 6.7 | Normal Breast | 28.7 |
| Ovarian Margin (OD06283-07) | 8.8 | Breast Cancer | 9.7 |
| Ovarian Cancer | 10.7 | Breast Cancer | 14.9 |
| Ovarian cancer (OD06145) | 4.2 | Breast Cancer (OD04590-01) | 32.5 |
| Ovarian Margin (OD06145) | 29.5 | Breast Cancer Mets | 12.2 |
| | | (OD04590-03) | |
| Ovarian cancer (OD06455-03) | 7.9 | Breast Cancer Metastasis | 25.5 |
| Ovarian Margin (OD06455-07) | 2.4 | Breast Cancer | 15.9 |
| Normal Lung | 26.2 | Breast Cancer 9100266 | 1.8 |
| Invasive poor diff. lung adeno | 8.3 | Breast Margin 9100265 | 1.6 |
| (ODO4945-01 | | | |
| Lung Margin (ODO4945-03) | 6.0 | Breast Cancer A209073 | 2.1 |
| Lung Malignant Cancer | 16.0 | Breast Margin A2090734 | 26.6 |
| (OD03126) | | | |
| Lung Margin (OD03126) | 5.5 | Breast cancer (OD06083) | 25.0 |
| Lung Cancer (OD05014A) | 7.0 | Breast cancer node metastasis | 27.5 |
| | | (OD06083) | |
| Lung Margin (OD05014B) | 7.3 | Normal Liver | 23.7 |
| Lung cancer (OD06081) | 20.4 | Liver Cancer 1026 | 2.7 |
| Lung Margin (OD06081) | 12.3 | Liver Cancer 1025 | 29.5 |
| Lung Cancer (OD04237-01) | 9.5 | Liver Cancer 6004-T | 24.0 |
| Lung Margin (OD04237-02) | 18.4 | Liver Tissue 6004-N | 23.2 |
| Ocular Mel Met to Liver | 14.1 | Liver Cancer 6005-T | 9.0 |
| (ODO4310) | | | |
| Liver Margin (ODO4310) | 15.0 | Liver Tissue 6005-N | 51.1 |
| Melanoma Metastasis | 12.5 | Liver Cancer | 31.4 |
| Lung Margin (OD04321) | 4.4 | Normal Bladder | 13.5 |
| Normal Kidney | 20.4 | Bladder Cancer | 6.4 |
| Kidney Ca, Nuclear grade 2 | 63.3 | Bladder Cancer | 11.3 |
| (OD04338) | | | |
| Kidney Margin (OD04338) | 11.5 | Normal Stomach | 48.6 |
| Kidney Ca Nuclear grade 1/2 | 46.3 | Gastric Cancer 9060397 | 5.6 |
| (OD04339) | | | |
| Kidney Margin (OD04339) | 17.4 | Stomach Margin 9060396 | 4.0 |
| Kidney Ca, Clear cell type | 24.8 | Gastric Cancer 9060395 | 4.0 |
| (OD04340) | | | |
| Kidney Margin (OD04340) | 17.0 | Stomach Margin 9060394 | 9.1 |
| Kidney Ca, Nuclear grade 3 | 3.1 | Gastric Cancer 064005 | 7.9 |
| (OD04348) | | | |

TABLE 71
Panel 4D

| | Rel. Exp. (%) | | Rel. Exp. (%) |
|---|---|---|---|
| | Ag2311, Run | | Ag2311, Run |
| Tissue Name | 158928074 | Tissue Name | 158928074 |
| Secondary Th1 act | 29.3 | HUVEC IL-1beta | 6.1 |
| Secondary Th2 act | 33.2 | HUVEC IFN gamma | 37.6 |
| Secondary Tr1 act | 45.7 | HUVEC TNF alpha + IFN | 36.3 |
| | | gamma | |
| Secondary Th1 rest | 17.3 | HUVEC TNF alpha + IL4 | 34.9 |
| Secondary Th2 rest | 19.2 | HUVEC IL-11 | 17.8 |
| Secondary Tr1 rest | 27.7 | Lung Microvascular EC none | 30.4 |
| Primary Th1 act | 36.9 | Lung Microvascular EC | 43.8 |
| | | TNF alpha + IL-1beta | |
| Primary Th2 act | 36.9 | Microvascular Dermal EC | 39.5 |
| | | none | |
| Primary Tr1 act | 45.7 | Microsvasular Dermal EC | 27.4 |
| | | TNF alpha + IL-1beta | |
| Primary Th1 rest | 38.7 | Bronchial epithelium | 1.2 |
| | | TNF alpha + IL1beta | |
| Primary Th2 rest | 27.0 | Small airway epithelium none | 6.2 |
| Primary Tr1 rest | 37.6 | Small airway epithelium | 20.0 |
| | | TNF alpha + IL-1beta | |
| CD45RA CD4 lymphocyte act | 17.2 | Coronery artery SMC rest | 9.5 |
| CD45RO CD4 lymphocyte act | 24.0 | Coronery artery SMC | 7.7 |
| | | TNF alpha + IL-1beta | |
| CD8 lymphocyte act | 21.9 | Astrocytes rest | 10.9 |
| Secondary CD8 lymphocyte rest | 26.2 | Astrocytes TNF alpha + IL- | 9.9 |
| | | 1beta | |
| Secondary CD8 lymphocyte act | 17.4 | KU-812 (Basophil) rest | 40.1 |
| CD4 lymphocyte none | 5.9 | KU-812 (Basophil) | 52.9 |
| | | PMA/ionomycin | |
| 2ry Th1/Th2/Tr1_anti-CD95 | 19.8 | CCD1106 (Keratinocytes) | 9.6 |
| CH11 | | none | |
| LAK cells rest | 32.8 | CCD1106 (Keratinocytes) | 0.6 |
| | | TNF alpha + IL-1beta | |
| LAK cells IL-2 | 23.3 | Liver cirrhosis | 10.8 |
| LAK cells IL-2 + IL-12 | 31.0 | Lupus kidney | 6.9 |
| LAK cells IL-2 + IFN gamma | 27.5 | NCI-H292 none | 65.1 |
| LAK cells IL-2 + IL-18 | 38.7 | NCI-H292 IL-4 | 65.1 |
| LAK cells PMA/ionomycin | 17.4 | NCI-H292 IL-9 | 71.7 |
| NK Cells IL-2 rest | 28.5 | NCI-H292 IL-13 | 41.2 |
| Two Way MLR 3 day | 36.9 | NCI-H292 IFN gamma | 43.8 |
| Two Way MLR 5 day | 19.5 | HPAEC none | 30.8 |
| Two Way MLR 7 day | 16.4 | HPAEC TNF alpha + IL-1 | 16.8 |
| | | beta | |
| PBMC rest | 23.5 | Lung fibroblast none | 25.7 |
| PBMC PWM | 35.1 | Lung fibroblast TNF alpha + | 9.0 |
| | | IL-1beta | |
| PBMC PHA-L | 17.8 | Lung fibroblast IL-4 | 43.5 |
| Ramos (B cell) none | 21.8 | Lung fibroblast IL-9 | 27.4 |
| Ramos (B cell) ionomycin | 37.4 | Lung fibroblast IL-13 | 29.1 |
| B lymphocytes PWM | 72.7 | Lung fibroblast LFN gamma | 23.5 |
| B lymphocytes CD40L and IL-4 | 66.0 | Dermal fibroblast CCD1070 | 42.3 |
| | | rest | |
| EOL-1 dbcAMP | 35.8 | Dermal fibroblast CCD1070 | 62.9 |
| | | TNF alpha | |
| EOL-1 dbcAMP | 38.4 | Dermal fibroblast CCD1070 | 28.3 |
| PMA/ionomycin | | IL-1beta | |
| Dendritic cells none | 59.0 | Dermal fibroblast IFN gamma | 18.3 |
| Dendritic cells LPS | 32.5 | Dermal fibroblast IL-4 | 33.2 |
| Dendritic cells anti-CD40 | 33.4 | IBD Colitis 2 | 5.3 |
| Monocytes rest | 42.9 | IBD Crohn's | 5.2 |
| Monocytes LPS | 25.0 | Colon | 27.5 |
| Macrophages rest | 42.3 | Lung | 14.5 |
| Macrophages LPS | 18.7 | Thymus | 47.6 |
| HUVEC none | 37.9 | Kidney | 100.0 |
| HUVEC starved | 40.1 | | |

CNS_Neurodegeneration_v1.0 Summary: Ag2311

NOV10 does not show differential expression between Alzheimer's diseased brains and control brains. However, this panel confirms the expression of this gene in the brains of an independent group of patients. Please see panel 1.3d for discussion of utility in the central nervous system.

Panel 1.3D Summary: Ag2311

NOV10, an alpha mannosidase isoform, is expressed at moderate levels in all regions of the brain examined, with highest expression in the substantia nigra (CT=29.3). In the brain, alpha mannosidase has been implicated in the processes of myelination and axon growth. Therefore, therapeutic modulation of this gene or its protein product may be of use in the treatment of disorders where myelination has been compromised such as multiple sclerosis, and schizophrenia. In addition, the protein encoded by NOV10 could be useful in clinical situations where increased axonal growth is desired including spinal cord or brain trauma, stroke, or peripheral nerve injury.

NOV10 gene is moderately expressed (CT values=31-34) in a variety of metabolic tissues including pancreas, adrenal, thyroid, pituitary, adult and fetal heart, adult and fetal liver, adult and fetal skeletal muscle, and adipose. This expression profile suggests that the protein encoded by the NOV10 may be an important small molecule target for the treatment of metabolic disease in any or all of these tissues, including obesity and diabetes.

The expression of this gene appears to be generally associated with normal tissues when compared to cell lines. Of note was the difference in expression in normal prostate when compared to the prostate cancer cell line (PC-3). Thus, NOV10 could be used to distinguish this sample on the panel from other samples or to distinguish normal prostate from prostate cancer. Moreover, therapeutic modulation of this gene, through the use of small molecule drugs, antibodies or protein therapetics, might be of use in the treatment of prostate cancer.

References:

1. Vite C H, McGowan J C, Braund K G, Drobatz K J, Glickson J D, Wolfe J H, Haskins M E. Histopathology, electrodiagnostic testing, and magnetic resonance imaging show significant peripheral and central nervous system myelin abnormalities in the cat model of alpha-mannosidosis. J Neuropathol Exp Neurol August 2001; 60(8):817-28

Alpha-mannosidosis is a disease caused by the deficient activity of alpha-mannosidase, a lysosomal hydrolase involved in the degradation of glycoproteins. The disease is characterized by the accumulation of mannose-rich oligosaccharides within lysosomes. The purpose of this study was to characterize the peripheral nervous system (PNS) and central nervous system (CNS) myelin abnormalities in cats from a breeding colony with a uniform mutation in the gene encoding alpha-mannosidase. Three affected cats and 3 normal cats from 2 litters were examined weekly from 4 to 18 wk of age. Progressively worsening neurological signs developed in affected cats that included tremors, loss of balance, and nystagmus. In the PNS, affected cats showed slow motor nerve conduction velocity and increased F-wave latency. Single nerve fiber teasing revealed significant demyelination/remyelination in affected cats. Mean G-ratios of nerves showed a significant increase in affected cats compared to normal cats. Magnetic resonance imaging of the CNS revealed diffuse white matter signal abnormalities throughout the brain of affected cats. Quantitative magnetization transfer imaging showed a 8%-16% decrease in the magnetization transfer ratio in brain white matter of affected cats compared to normal cats, consistent with myelin abnormalities. Histology confirmed myelin loss throughout the cerebrum and cerebellum. Thus, histology, electrodiagnostic testing, and magnetic resonance imaging identified significant myelination abnormalities in both the PNS and CNS that have not been described previously in alpha-mannosidosis.

2. Zmuda J F, Rivas R J. The Golgi apparatus and the centrosome are localized to the sites of newly emerging axons in cerebellar granule neurons in vitro. Cell Motil Cytoskeleton 1998;41(1):18-38

Cultured cerebellar granule neurons develop their characteristic axonal and dendritic morphologies in a series of discrete temporal steps highly similar to those observed in situ, initially extending a single process, followed by the extension of a second process from the opposite pole of the cell, both of which develop into axons to generate a bipolar morphology. A mature morphology is attained following the outgrowth of multiple, short dendrites [Powell et al., 1997: J. Neurobiol. 32:223-236]. To determine the relationship between the localization of the Golgi apparatus, the site of microtubule nucleation (the centrosome), and the sites of initial and secondary axonal extension, the intracellular positioning of the Golgi and centrosome was observed during the differentiation of postnatal mouse granule neurons in vitro. The Golgi was labeled using the fluorescent lipid analogue, C5-DMB-Ceramide, or by indirect immunofluorescence using antibodies against the Golgi resident protein, alpha-mannosidase II. At 1-2 days in vitro (DIV), the Golgi was positioned at the base of the initial process in 99% of unipolar cells observed. By 3 DIV, many cells began the transition to a bipolar morphology by extending a short neurite from the pole of the cell opposite to the initial process. The Golgi was observed at this site of secondary outgrowth in 92% of these “transitional” cells, suggesting that the Golgi was repositioned from the base of the initial process to the site of secondary neurite outgrowth. As the second process elongated and the cells proceeded to the bipolar stage of development, or at later stages when distinct axonal and somatodendritic domains had been established, the Golgi was not consistently positioned at the base of either axons or dendrites, and was most often found at sites on the plasma membrane from which no processes originated. To determine the location of the centrosome in relation to the Golgi during development, granule neurons were labeled with antibodies against gamma-tubulin and optically sectioned using confocal microscopy. The centrosome was consistently co-localized with the Golgi during all stages of differentiation, and also appeared to be repositioned to the base of the newly emerging axon during the transition from a unipolar to a bipolar morphology. These findings indicate that during the early stages of granule cell axonal outgrowth, the Golgi-centrosome is positioned at the base of the initial axon and is then repositioned to the base of the newly emerging secondary axon. Such an intracellular reorientation of these organelles may be important in maintaining the characteristic developmental pattern of granule neurons by establishing the polarized microtubule network and the directed flow of membranous vesicles required for initial axonal elaboration

Panel 2.2 Summary: Ag2311

The expression of this gene is highest in a sample derived from normal kidney tissue adjacent to a kidney cancer. Furthermore, there appears to be substantial expression in normal stomach, normal liver adjacent to a cancer, normal breast adjacent to a cancer and normal ovary adjacent to a cancer. Thus, the expression of this gene could be used to distinguish these normal tissues from their malignant counterparts. Moreover, therapeutic modulation of this gene, through the use of small molecule durgs, antibodies or protein therapeutics might be of use in the treatment of kidney, liver, breast or ovarian cancer.

Panel 4D Summary: Ag2311

NOV10 is modestly expressed (CT values=30-33) in a wide variety of immune cell types and tissues. The highest expression of this gene is found in B cells stimulated with PWM and anti-CD40, where stimulation normally leads to the production of immunoglobulin (Ig) and Ig switching. High levels of expression of this transcript are also found in a pulmonary muco-epidermoid cell line (H292) treated with Th2 cytokines. These findings suggest that the NOV10 product may be important in the pathogenesis, and/or treatment of autoimmune diseases such as lupus erythematosus, rheumatoid arthritis, inflammatory bowel disease, allergies which are associated with hyper IgE production, and lung inflammatory diseases such as asthma and emphysema. In addition, the high expression of this gene in the kidney suggests that the protein encoded by this transcript may be involved in normal tissue/cellular functions particularly in the kidney.

NOV11a, NOV11b

Expression of NOV11a and NOV11b was assessed using the primer-probe set Ag3670, described in Table 72. Results of the RTQ-PCR runs are shown in Tables 73 and 74.

TABLE 72

Probe Name Ag3670

| | | | | |
|---|---|---|---|---|
| | | | | SEQ |
| | | | Start | ID |
| Primers | Sequences | Length | Position | NO: |

| | | | | |
|---|---|---|---|---|
| Forward | 5′-ACGAGGTCTTCATCAAGCTG- | 20 | 705 | 187 |
| | 3′ | | | |
| Probe | TET-5′-CACCAACAAGTACAGC- | 26 | 751 | 188 |
| | ACCTTCTCCG-3′-TAMRA | | | |
| Reverse | 5′-CAGTCGGGGTAGATGATGAA- | 20 | 779 | 189 |
| | 3′ | | | |

TABLE 73
General_screening_panel_v1.4

| | Rel. | Rel. | | Rel. | Rel. |
|---|---|---|---|---|---|
| | Exp. (%) | Exp. (%) | | Exp. (%) | Exp. (%) |
| | Ag3670, | Ag3670, | | Ag3670, | Ag3670, |
| | Run | Run | | Run | Run |
| Tissue Name | 216517130 | 222735036 | Tissue Name | 216517130 | 222735036 |
| Adipose | 0.2 | 0.4 | Renal ca. TK-10 | 2.1 | 1.1 |
| Melanoma* | 0.0 | 0.0 | Bladder | 0.4 | 0.0 |
| Hs688(A).T | | | | | |
| Melanoma* | 0.0 | 0.0 | Gastric ca. (liver met.) | 0.8 | 0.4 |
| Hs688(B).T | | | NCI-N87 | | |
| Melanoma* M14 | 6.0 | 6.9 | Gastric ca. KATO III | 0.4 | 0.4 |
| Melanoma* | 2.6 | 1.8 | Colon ca. SW-948 | 0.0 | 0.1 |
| LOXIMVI | | | | | |
| Melanoma* SK- | 5.9 | 8.2 | Colon ca. SW480 | 2.6 | 3.5 |
| MEL-5 | | | | | |
| Squamous cell | 0.2 | 0.0 | Colon ca.* (SW480 | 1.3 | 0.8 |
| carcinoma SCC-4 | | | met) SW620 | | |
| Testis Pool | 3.2 | 1.5 | Colon ca. HT29 | 0.1 | 0.0 |
| Prostate ca.* (bone | 11.0 | 11.7 | Colon ca. HCT-116 | 27.0 | 25.9 |
| met) PC-3 | | | | | |
| Prostate Pool | 0.0 | 0.1 | Colon ca. CaCo-2 | 11.7 | 13.3 |
| Placenta | 0.0 | 0.0 | Colon cancer tissue | 0.0 | 0.3 |
| Uterus Pool | 0.1 | 0.1 | Colon ca. SW1116 | 12.9 | 7.2 |
| Ovarian ca. | 2.3 | 1.2 | Colon ca. Colo-205 | 0.4 | 0.2 |
| OVCAR-3 | | | | | |
| Ovarian ca. SK-OV-3 | 1.9 | 3.2 | Colon ca. SW-48 | 0.0 | 0.3 |
| Ovarian ca. | 1.5 | 1.5 | Colon Pool | 0.0 | 0.4 |
| OVCAR-4 | | | | | |
| Ovarian ca. | 6.3 | 6.0 | Small Intestine Pool | 0.0 | 0.0 |
| OVCAR-5 | | | | | |
| Ovarian ca. IGROV-1 | 0.6 | 1.6 | Stomach Pool | 0.1 | 0.2 |
| Ovarian ca. | 6.9 | 7.9 | Bone Marrow Pool | 0.2 | 0.1 |
| OVCAR-8 | | | | | |
| Ovary | 0.0 | 0.0 | Fetal Heart | 0.0 | 0.0 |
| Breast ca. MCF-7 | 1.5 | 2.7 | Heart Pool | 0.0 | 0.0 |
| Breast ca. MDA- | 0.8 | 0.6 | Lymph Node Pool | 0.2 | 0.2 |
| MB-231 | | | | | |
| Breast ca. BT 549 | 6.8 | 11.6 | Fetal Skeletal Muscle | 0.0 | 0.0 |
| Breast ca. T47D | 21.8 | 16.8 | Skeletal Muscle Pool | 0.0 | 0.0 |
| Breast ca. MDA-N | 4.0 | 2.8 | Spleen Pool | 0.0 | 0.0 |
| Breast Pool | 0.0 | 0.0 | Thymus Pool | 0.3 | 0.1 |
| Trachea | 0.0 | 0.3 | CNS cancer (glio/astro) | 0.5 | 1.2 |
| | | | U87-MG | | |
| Lung | 0.0 | 0.0 | CNS cancer (glio/astro) | 3.2 | 3.2 |
| | | | U-118-MG | | |
| Fetal Lung | 0.0 | 0.0 | CNS cancer | 6.4 | 10.0 |
| | | | (neuro; met) SK-N-AS | | |
| Lung ca. NCI-N417 | 5.7 | 4.5 | CNS cancer (astro) SF- | 2.6 | 0.8 |
| | | | 539 | | |
| Lung ca. LX-1 | 0.2 | 0.5 | CNS cancer (astro) | 0.9 | 1.6 |
| | | | SNB-75 | | |
| Lung ca. NCI-H146 | 0.5 | 0.8 | CNS cancer (glio) | 0.3 | 0.8 |
| | | | SNB-19 | | |
| Lung ca. SHP-77 | 0.6 | 2.7 | CNS cancer (glio) SF- | 4.9 | 4.4 |
| | | | 295 | | |
| Lung ca. A549 | 6.8 | 6.6 | Brain (Amygdala) Pool | 0.0 | 0.3 |
| Lung ca. NCI-H526 | 1.6 | 2.5 | Brain (cerebellum) | 0.0 | 0.3 |
| Lung ca. NCI-H23 | 16.4 | 12.7 | Brain (fetal) | 2.0 | 2.7 |
| Lung ca. NCI-H460 | 0.3 | 0.0 | Brain (Hippocampus) | 0.3 | 0.8 |
| | | | Pool | | |
| Lung ca. HOP-62 | 0.6 | 0.8 | Cerebral Cortex Pool | 0.0 | 0.2 |
| Lung ca. NCI-H522 | 48.6 | 46.0 | Brain (Substantia nigra) | 0.4 | 0.7 |
| | | | Pool | | |
| Liver | 0.0 | 0.0 | Brain (Thalamus) Pool | 0.4 | 0.9 |
| Fetal Liver | 0.0 | 0.0 | Brain (whole) | 0.1 | 0.1 |
| Liver ca. HepG2 | 0.1 | 0.3 | Spinal Cord Pool | 0.2 | 0.3 |
| Kidney Pool | 0.1 | 0.0 | Adrenal Gland | 0.0 | 0.0 |
| Fetal Kidney | 1.0 | 1.9 | Pituitary gland Pool | 0.0 | 0.3 |
| Renal ca. 786-0 | 100.0 | 100.0 | Salivary Gland | 0.0 | 0.2 |
| Renal ca. A498 | 10.2 | 21.3 | Thyroid (female) | 1.2 | 0.3 |
| Renal ca. ACHN | 0.5 | 0.2 | Pancreatic ca. | 0.9 | 0.2 |
| | | | CAPAN2 | | |
| Renal ca. UO-31 | 1.5 | 1.8 | Pancreas Pool | 0.0 | 0.2 |

TABLE 74
Panel 4.1D

| | Rel. | | Rel. |
|---|---|---|---|
| | Exp. (%) | | Exp. (%) |
| | Ag3670, | | Ag3670, |
| | Run | | Run |
| Tissue Name | 223785547 | Tissue Name | 223785547 |
| Secondary Th1 act | 0.0 | HUVEC IL-1beta | 27.5 |
| Secondary Th2 act | 0.0 | HUVEC IFN gamma | 29.9 |
| Secondary Tr1 act | 0.0 | HUVEC TNF alpha + IFN gamma | 19.5 |
| Secondary Th1 rest | 0.0 | HUVEC TNF alpha + IL4 | 0.0 |
| Secondary Th2 rest | 0.0 | HUVEC IL-11 | 0.0 |
| Secondary Tr1 rest | 0.0 | Lung Microvascular EC none | 0.0 |
| Primary Th1 act | 0.0 | Lung Microvascular EC | 11.6 |
| | | TNFalpha + IL-1beta | |
| Primary Th2 act | 0.0 | Microvascular Dermal EC none | 0.0 |
| Primary Tr1 act | 0.0 | Microsvasular Dermal EC | 71.7 |
| | | TNFalpha + IL-1beta | |
| Primary Th1 rest | 0.0 | Bronchial epithelium TNFalpha + | 53.2 |
| | | IL1beta | |
| Primary Th2 rest | 0.0 | Small airway epithelium none | 0.0 |
| Primary Tr1 rest | 0.0 | Small airway epithelium | 0.0 |
| | | TNFalpha + IL-1beta | |
| CD45RA CD4 lymphocyte act | 0.0 | Coronery artery SMC rest | 0.0 |
| CD45RO CD4 lymphocyte act | 0.0 | Coronery artery SMC TNFalpha + | 0.0 |
| | | IL-1beta | |
| CD8 lymphocyte act | 15.1 | Astrocytes rest | 0.0 |
| Secondary CD8 lymphocyte rest | 0.0 | Astrocytes TNFalpha + IL-1beta | 0.0 |
| Secondary CD8 lymphocyte act | 39.5 | KU-812 (Basophil) rest | 0.0 |
| CD4 lymphocyte none | 0.0 | KU-812 (Basophil) | 0.0 |
| | | PMA/ionomycin | |
| 2ry Th1/Th2/Tr1_anti-CD95 | 0.0 | CCD1106 (Keratinocytes) none | 0.0 |
| CH11 | | | |
| LAK cells rest | 0.0 | ICCD 1106 (Keratinocytes)I | 38.2 |
| | | TNFalpha + IL-1beta | |
| LAK cells IL-2 | 0.0 | Liver cirrhosis | 0.0 |
| LAK cells IL-2 + IL-12 | 0.0 | NCI-H292 none | 0.0 |
| LAK cells IL-2 + IFN gamma | 0.0 | NCI-H292 IL-4 | 34.9 |
| LAK cells IL-2 + IL-18 | 0.0 | NCI-H292 IL-9 | 24.8 |
| LAK cells PMA/ionomycin | 0.0 | NCI-H292 IL-13 | 21.5 |
| NK Cells IL-2 rest | 0.0 | NCI-H292 IFN gamma | 33.9 |
| Two Way MLR 3 day | 0.0 | HPAEC none | 0.0 |
| Two Way MLR 5 day | 0.0 | HPAEC TNF alpha + IL-1 beta | 0.0 |
| Two Way MLR 7 day | 0.0 | Lung fibroblast none | 12.8 |
| PBMC rest | 0.0 | Lung fibroblast TNF alpha + IL-1 | 0.0 |
| | | beta | |
| PBMC PWM | 0.0 | Lung fibroblast IL-4 | 29.3 |
| PBMC PHA-L | 0.0 | Lung fibroblast IL-9 | 46.7 |
| Ramos (B cell) none | 0.0 | Lung fibroblast IL-13 | 0.0 |
| Ramos (B cell) ionomycin | 0.0 | Lung fibroblast IFN gamma | 0.0 |
| B lymphocytes PWM | 0.0 | Dermal fibroblast CCD1070 rest | 24.5 |
| B lymphocytes CD40L and IL-4 | 0.0 | Dermal fibroblast CCD1070 TNF | 22.8 |
| | | alpha | |
| EOL-1 dbcAMP | 100.0 | Dermal fibroblast CCD 1070 IL-1 | 0.0 |
| | | beta | 0.0 |
| EOL-1 dbcAMP | 45.1 | Dermal fibroblast IFN gamma | 0.0 |
| PMA/ionomycin | | | |
| Dendritic cells none | 0.0 | Dermal fibroblast IL-4 | 0.0 |
| Dendritic cells LPS | 0.0 | Dermal Fibroblasts rest | 0.0 |
| Dendritic cells anti-CD40 | 0.0 | Neutrophils TNFa + LPS | 0.0 |
| Monocytes rest | 0.0 | Neutrophils rest | 0.0 |
| Monocytes LPS | 0.0 | Colon | 0.0 |
| Macrophages rest | 0.0 | Lung | 0.0 |
| Macrophages LPS | 0.0 | Thymus | 0.0 |
| HUVEC none | 25.9 | Kidney | 59.5 |
| HUVEC starved | 0.0 | | |

General_Screening_Panel_v1.4 Summary: Ag3670

Two experiments with the same probe and primer sets show results that are in excellent agreement, with highest expression in a renal cancer cell line. In general, the expression of this gene appears to be largely associated with samples derived from cancer cell lines rather than normal tissues. Of note is the substantial expression associated with kidney cancer cell lines as well as in colon cancer and lung cancer cell lines. Thus, the expression of this gene could be used to distinguish these cell lines from other cell lines. Moreover, therapeutic modulation of this gene, through the use of small molecule drugs, antibodies or protein therapeutics might be of use in the treatment of kidney, colon or lung cancer.

This gene is a C1q-related factor variant, and is expressed in at least the fetal brain, hippocampus, substantia nigra and thalamus. Various members of the complement cascade have been implicated in neuroinflammation and the pathology of Alzheimer's disease. Recent case controlled studies also suggest that the use of anti-inflammatory agents decreases the risk of Alzheimer's disease. Therefore, this gene is an excellent drug target for the disruption of neuroinflammation and the treatment of Alzheimer's disease, Huntington's disease, and stroke.

References:

Lue L F, Rydel R, Brigham E F, Yang L B, Hampel H, Murphy G M Jr, Brachova L, Yan S D, Walker D G, Shen Y, Rogers J. Inflammatory repertoire of Alzheimer's disease and nondemented elderly microglia in vitro. Glia July 2001; 35(1):72-9

In this study complement activation and biosynthesis have been analysed in the brains of Huntington's disease (HD) (n=9) and normal (n=3) individuals. In HD striatum, neurons, myelin and astrocytes were strongly stained with antibodies to C1q, C4, C3, iC3b-neoepitope and C9-neoepitope. In contrast, no staining for complement components was found in the normal striatum. Marked astrogliosis and microgliosis were observed in all HD caudate and the internal capsule samples but not in normal brain. RT-PCR analysis and in-situ hybridisation were carried out to determine whether complement was synthesised locally by activated glial cells. By RT-PCR, we found that complement activators of the classical pathway C1q C chain, C1r, C4, C3, as well as the complement regulators, C1 inhibitor, clusterin, MCP, DAF, CD59, were all expressed constitutively and at much higher level in HD brains compared to normal brain. Complement anaphylatoxin receptor mRNAs (C5a receptor and C3a receptor) were strongly expressed in HD caudate. In general, we found that the level of complement mRNA in normal control brains was from 2 to 5 fold lower compared to HD striatum. Using in-situ hybridisation, we confirmed that C3 mRNA and C9 mRNA were expressed by reactive microglia in HD internal capsule. We propose that complement produced locally by reactive microglia is activated on the membranes of neurons, contributing to neuronal necrosis but also to proinflammatory activities. Complement opsonins (iC3b) and anaphylatoxins (C3a, C5a) may be involved in the recruitment and stimulation of glial cells and phagocytes bearing specific complement receptors.

Panel 4.1D Summary: Ag3670

The NOV11 transcript, which encodes a protein with homology to a C1q related factor, is expressed at a low level in eosinophils, microvascular dermal endothelial cells and bronchial epithelium. The inflammatory cytokines TNF-a and IL-1b appear to up-regulate expression of this transcript in the endothelial cells and bronchial epithelium. This suggests that expression of this transcript is regulated by inflammatory conditions such as those found in lung inflammatory disease including pneumonia and bronchitis as well as skin infection or wounds. Expression of this transcript is also up regulated in lung fibroblasts by the Th2 cytokines IL9 or IL4, conditions found in asthma and COPD. The expression of this transcript in eosinophils, cells that are frequently associated with asthma, ulcerative colitis or other Th2 mediated diseases strongly suggest that modulation of the expression of this transcript will be beneficial in the treatment of atopic lung and skin diseases. Since the C1q factor is usually involved in the activation of complement and innate immunity, modulation of the expression of this transcript could modulate excessive inflammatory processes leading to these diseases.

Panel 5D Summary: Expression is low/undetectable for all samples in this panel (CT>35). (Data not shown).

NOV12

Expression of NOV12 was assessed using the primer-probe sets Ag1586 and Ag2011, described in Tables 75 and 76. Results of the RTQ-PCR runs are shown in Tables 77, 78, 79 and 80.

TABLE 75

Probe Name Ag1586

| | | | | SEQ |
|---|---|---|---|---|
| | | | Start | ID |
| Primers | Sequences | Length | Position | NO: |
| Forward | 5′-ACCAGGATGAGTTTGTGTCA- | 22 | 735 | 190 |
| | TC-3′ | | | |
| Probe | TET-5′-CTCAAGATCCCTTCGG- | 25 | 761 | 191 |
| | ACACGCTGT-3′-TAMRA | | | |
| Reverse | 5′-TGCGGAAGCTGTACACATAG- | 22 | 809 | 192 |
| | TA-3′ | | | |

TABLE 76
Probe Name Ag2011

| | | | | |
|---|---|---|---|---|
| | | | | SEQ |
| | | | Start | ID |
| Primers | Sequences | Length | Position | NO: |

| | | | | |
|---|---|---|---|---|
| Forward | 5′-ACCAGGATGAGTTTGTGTCA- | 22 | 735 | 193 |
| | TC-3′ | | | |
| Probe | TET-5′-CTCAAGATCCCTTCGG- | 25 | 761 | 194 |
| | ACACGCTGT-3′-TAMRA | | | |
| Reverse | 5′-TGCGGAAGCTGTACACATAG- | 22 | 809 | 195 |
| | TA-3′ | | | |

TABLE 77
Panel 1.3D

| | Rel. | Rel. | | Rel. | Rel. |
|---|---|---|---|---|---|
| | Exp. (%) | Exp. (%) | | Exp. (%) | Exp. (%) |
| | Ag1586, | Ag2011, | | Ag1586, | Ag2011, |
| | Run | Run | | Run | Run |
| Tissue Name | 146473155 | 147816085 | Tissue Name | 146473155 | 147816085 |
| Liver adenocarcinoma | 29.9 | 37.6 | Kidney (fetal) | 3.8 | 3.7 |
| Pancreas | 1.7 | 0.7 | Renal ca. 786-0 | 6.1 | 11.7 |
| Pancreatic ca. CAPAN 2 | 6.3 | 9.6 | Renal ca. A498 | 25.0 | 25.9 |
| Adrenal gland | 2.6 | 2.5 | Renal ca. RXF 393 | 4.5 | 5.0 |
| Thyroid | 2.5 | 1.8 | Renal ca. ACHN | 8.8 | 11.3 |
| Salivary gland | 1.9 | 2.2 | Renal ca. UO-31 | 15.0 | 15.0 |
| Pituitary gland | 0.9 | 1.5 | Renal ca. TK-10 | 4.4 | 4.6 |
| Brain (fetal) | 12.2 | 13.1 | Liver | 0.2 | 0.1 |
| Brain (whole) | 9.7 | 10.7 | Liver (fetal) | 0.7 | 0.8 |
| Brain (amygdala) | 9.5 | 9.9 | Liver ca. | 16.8 | 12.8 |
| | | | (hepatoblast) | | |
| | | | HepG2 | | |
| Brain (cerebellum) | 3.3 | 2.3 | Lung | 5.0 | 5.1 |
| Brain (hippocampus) | 24.7 | 21.0 | Lung (fetal) | 7.4 | 8.1 |
| Brain (substantia nigra) | 0.9 | 1.3 | Lung ca. (small | 16.8 | 12.1 |
| | | | cell) LX-1 | | |
| Brain (thalamus) | 4.7 | 3.7 | Lung ca. (small | 18.4 | 23.7 |
| | | | cell) NCI-H69 | | |
| Cerebral Cortex | 75.8 | 71.2 | Lung ca. (s.cell | 8.5 | 7.2 |
| | | | var.) SHP-77 | | |
| Spinal cord | 2.0 | 2.4 | Lung ca. (large | 10.7 | 10.1 |
| | | | cell)NCI-H460 | | |
| glio/astro U87-MG | 15.3 | 17.9 | Lung ca. (non-sm. | 3.2 | 4.1 |
| | | | cell) A549 | | |
| glio/astro U-118-MG | 38.2 | 41.2 | Lung ca. (non- | 23.2 | 24.7 |
| | | | s.cell) NCI-H23 | | |
| astrocytoma SW1783 | 8.3 | 10.4 | Lung ca. (non- | 18.9 | 15.7 |
| | | | s.cell) HOP-62 | | |
| neuro*; met SK-N-AS | 23.5 | 24.3 | Lung ca. (non-s.cl) | 5.6 | 7.5 |
| | | | NCI-H522 | | |
| astrocytoma SF-539 | 19.6 | 38.4 | Lung ca. (squam.) | 13.0 | 13.1 |
| | | | SW 900 | | |
| astrocytoma SNB-75 | 44.4 | 45.1 | Lung ca. (squam.) | 6.5 | 5.7 |
| | | | NCI-H596 | | |
| glioma SNB-19 | 26.2 | 12.2 | Mammary gland | 11.5 | 9.3 |
| glioma U251 | 16.4 | 16.2 | Breast ca.* (pl.ef) | 14.1 | 14.4 |
| | | | MCF-7 | | |
| glioma SF-295 | 26.4 | 36.9 | Breast ca.* (pl.ef) | 82.9 | 87.1 |
| | | | MDA-MB-231 | | |
| Heart (Fetal) | 80.7 | 95.3 | Breast ca.* (pl.ef) | 6.1 | 4.6 |
| | | | T47D | | |
| Heart | 2.8 | 1.9 | Breast ca. BT-549 | 13.6 | 11.2 |
| Skeletal muscle (Fetal) | 85.3 | 87.7 | Breast ca. MDA-N | 28.1 | 31.6 |
| Skeletal muscle | 2.1 | 2.4 | Ovary | 20.9 | 19.5 |
| Bone marrow | 0.6 | 0.3 | Ovarian ca. | 33.0 | 40.1 |
| | | | OVCAR-3 | | |
| Thymus | 2.6 | 2.3 | Ovarian ca. | 5.5 | 5.4 |
| | | | OVCAR-4 | | |
| Spleen | 2.9 | 2.6 | Ovarian ca. | 10.9 | 13.1 |
| | | | OVCAR-5 | | |
| Lymph node | 5.1 | 5.2 | Ovarian ca. | 17.4 | 18.3 |
| | | | OVCAR-8 | | |
| Colorectal | 5.2 | 3.9 | Ovarian ca. | 4.5 | 5.3 |
| | | | IGROV-1 | | |
| Stomach | 3.7 | 5.6 | Ovarian ca. | 25.7 | 22.4 |
| | | | (ascites) SK-OV-3 | | |
| Small intestine | 1.6 | 1.3 | Uterus | 2.7 | 2.4 |
| Colon ca. SW480 | 45.4 | 55.5 | Placenta | 6.7 | 10.2 |
| Colon ca.* SW620 | 11.3 | 11.1 | Prostate | 0.4 | 1.4 |
| (SW480 met) | | | | | |
| Colon ca. HT29 | 13.3 | 13.3 | Prostate ca.* (bone | 8.4 | 11.3 |
| | | | met) PC-3 | | |
| Colon ca. HCT-116 | 10.5 | 10.5 | Testis | 8.1 | 8.5 |
| Colon ca. CaCo-2 | 24.0 | 23.0 | Melanoma | 59.0 | 86.5 |
| | | | Hs688(A).T | | |
| CC Well to Mod Diff | 19.1 | 16.6 | Melanoma* (met) | 100.0 | 100.0 |
| (ODO3866) | | | Hs688(B).T | | |
| Colon ca. HCC-2998 | 25.7 | 20.3 | Melanoma UACC- | 17.6 | 19.5 |
| | | | 62 | | |
| Gastric ca. (liver met) | 59.9 | 62.9 | Melanoma M14 | 16.3 | 21.9 |
| NCI-N87 | | | | | |
| Bladder | 1.8 | 4.6 | Melanoma LOX | 3.6 | 5.8 |
| | | | IMVI | | |
| Trachea | 6.9 | 5.6 | Melanoma* (met) | 12.9 | 22.1 |
| | | | SK-MEL-5 | | |
| Kidney | 0.8 | 0.7 | Adipose | 5.6 | 4.5 |

TABLE 78
Panel 2.2

| | Rel. | | Rel. |
|---|---|---|---|
| | Exp. (%) | | Exp. (%) |
| | Ag2011, | | Ag2011, |
| | Run | | Run |
| Tissue Name | 174154748 | Tissue Name | 174154748 |
| Normal Colon | 24.7 | Kidney Margin (OD04348) | 68.3 |
| Colon cancer (OD06064) | 48.6 | Kidney malignant cancer | 25.0 |
| | | (OD06204B) | |
| Colon Margin (OD06064) | 4.9 | Kidney normal adjacent tissue | 7.4 |
| | | (OD06204E) | |
| Colon cancer (OD06159) | 9.3 | Kidney Cancer (OD04450-01) | 34.4 |
| Colon Margin (OD06159) | 19.5 | Kidney Margin (OD04450-03) | 18.4 |
| Colon cancer (OD06297-04) | 11.7 | Kidney Cancer 8120613 | 9.7 |
| Colon Margin (OD06297-015) | 12.5 | Kidney Margin 8120614 | 18.8 |
| CC Gr. 2 ascend colon | 17.3 | Kidney Cancer 9010320 | 16.2 |
| (OD03921) | | | |
| CC Margin (OD03921) | 14.2 | Kidney Margin 9010321 | 13.8 |
| Colon cancer metastasis | 8.6 | Kidney Cancer 8120607 | 37.1 |
| (OD06104) | | | |
| Lung Margin (OD06104) | 8.3 | Kidney Margin 8120608 | 7.0 |
| Colon mets to lung (OD04451- | 23.0 | Normal Uterus | 21.9 |
| 01) | | | |
| Lung Margin (OD04451-02) | 32.8 | Uterine Cancer 064011 | 13.7 |
| Normal Prostate | 4.8 | Normal Thyroid | 2.4 |
| Prostate Cancer (OD04410) | 4.9 | Thyroid Cancer | 8.1 |
| Prostate Margin (OD04410) | 8.8 | Thyroid Cancer A302152 | 35.4 |
| Normal Ovary | 32.3 | Thyroid Margin A302153 | 8.7 |
| Ovarian cancer (OD06283-03) | 32.1 | Normal Breast | 29.7 |
| Ovarian Margin (OD06283-07) | 13.8 | Breast Cancer | 11.9 |
| Ovarian Cancer | 19.9 | Breast Cancer | 47.6 |
| Ovarian cancer (OD06145) | 9.2 | Breast Cancer (OD04590-01) | 25.5 |
| Ovarian Margin (OD06145) | 8.6 | Breast Cancer Mets (OD04590- | 38.4 |
| | | 03) | |
| Ovarian cancer (OD06455-03) | 13.0 | Breast Cancer Metastasis | 30.1 |
| Ovarian Margin (OD06455-07) | 2.1 | Breast Cancer | 41.5 |
| Normal Lung | 27.2 | Breast Cancer 9100266 | 9.2 |
| Invasive poor diff. lung adeno | 28.5 | Breast Margin 9100265 | 18.2 |
| (ODO4945-01 | | | |
| Lung Margin (ODO4945-03) | 15.0 | Breast Cancer A209073 | 14.9 |
| Lung Malignant Cancer | 30.4 | Breast Margin A2090734 | 37.6 |
| (OD03126) | | | |
| Lung Margin (OD03126) | 15.9 | Breast cancer (OD06083) | 55.9 |
| Lung Cancer (OD05014A) | 39.5 | Breast cancer node metastasis | 48.6 |
| | | (OD06083) | |
| Lung Margin (OD05014B) | 22.1 | Normal Liver | 10.4 |
| Lung cancer (OD06081) | 23.7 | Liver Cancer 1026 | 9.1 |
| Lung Margin (OD06081) | 16.8 | Liver Cancer 1025 | 20.7 |
| Lung Cancer (OD04237-01) | 9.0 | Liver Cancer 6004-T | 12.2 |
| Lung Margin (OD04237-02) | 41.5 | Liver Tissue 6004-N | 8.0 |
| Ocular Mel Met to Liver | 100.0 | Liver Cancer 6005-T | 36.6 |
| (ODO4310) | | | |
| Liver Margin (ODO4310) | 4.2 | Liver Tissue 6005-N | 25.0 |
| Melanoma Metastasis | 47.0 | Liver Cancer | 4.5 |
| Lung Margin (OD04321) | 28.1 | Normal Bladder | 18.7 |
| Normal Kidney | 12.3 | Bladder Cancer | 17.2 |
| Kidney Ca, Nuclear grade 2 | 18.3 | Bladder Cancer | 72.7 |
| (OD04338) | | | |
| Kidney Margin (OD04338) | 18.0 | Normal Stomach | 33.4 |
| Kidney Ca Nuclear grade ½ | 83.5 | Gastric Cancer 9060395 | 9.6 |
| (OD04339) | | | |
| Kidney Margin (OD04339) | 10.4 | Stomach Margin 9060396 | 10.4 |
| Kidney Ca, Clear cell type | 22.2 | Gastric Cancer 9060395 | 7.6 |
| (OD04340) | | | |
| Kidney Margin (OD04340) | 12.7 | Stomach Margin 9060394 | 19.6 |
| Kidney Ca, Nuclear grade 3 | 15.7 | Gastric Cancer 064005 | 17.4 |
| (OD04348) | | | |

TABLE 79
Panel 2D

| | Rel. | | Rel. |
|---|---|---|---|
| | Exp. (%) | | Exp. (%) |
| | Ag1586, | | Ag1586, |
| | Run | | Run |
| Tissue Name | 162624476 | Tissue Name | 162624476 |
| Normal Colon | 34.9 | Kidney Margin 8120608 | 14.2 |
| CC Well to Mod Diff | 28.3 | Kidney Cancer 8120613 | 30.4 |
| (ODO3866) | | | |
| CC Margin (ODO3866) | 9.2 | Kidney Margin 8120614 | 17.7 |
| CC Gr. 2 rectosigmoid | 25.9 | Kidney Cancer 9010320 | 57.0 |
| (ODO3868) | | | |
| CC Margin (ODO3868) | 4.7 | Kidney Margin 9010321 | 40.9 |
| CC Mod Diff (ODO3920) | 55.5 | Normal Uterus | 10.4 |
| CC Margin (ODO3920) | 14.2 | Uterine Cancer 064011 | 28.9 |
| CC Gr. 2 ascend colon | | | |
| (ODO3921) | 62.9 | Normal Thyroid | 8.4 |
| CC Margin (ODO3921) | 12.1 | Thyroid Cancer | 16.7 |
| CC from Partial Hepatectomy | 41.5 | Thyroid Cancer A302152 | 24.7 |
| (ODO4309) Mets | | | |
| Liver Margin (ODO4309) | 13.6 | Thyroid Margin A302153 | 17.7 |
| Colon mets to lung (OD04451- | 18.0 | Normal Breast | 60.3 |
| 01) | | | |
| Lung Margin (OD04451-02) | 25.5 | Breast Cancer | 24.1 |
| Normal Prostate 6546-1 | 17.0 | Breast Cancer (OD04590-01) | 47.0 |
| Prostate Cancer (OD04410) | 33.7 | Breast Cancer Mets (OD059O- | 72.7 |
| | | 03) | |
| Prostate Margin (OD04410) | 28.9 | Breast Cancer Metastasis | 37.4 |
| Prostate Cancer (OD04720-01) | 33.7 | Breast Cancer | 36.9 |
| Prostate Margin (OD04720-02) | 45.7 | Breast Cancer | 65.1 |
| Normal Lung | 80.7 | Breast Cancer 9100266 | 39.8 |
| Lung Met to Muscle (ODO4286) | 100.0 | Breast Margin 9100265 | 31.2 |
| Muscle Margin (ODO4286) | 21.5 | Breast Cancer A209073 | 49.0 |
| Lung Malignant Cancer | 57.8 | Breast Margin A2090734 | 44.8 |
| (OD03126) | | | |
| Lung Margin (OD03126) | 61.6 | Normal Liver | 4.5 |
| Lung Cancer (OD04404) | 70.2 | Liver Cancer | 2.6 |
| Lung Margin (OD04404) | 34.2 | Liver Cancer 1025 | 4.7 |
| Lung Cancer (OD04565) | 87.7 | Liver Cancer 1026 | 18.3 |
| Lung Margin (OD04565) | 23.8 | Liver Cancer 6004-T | 7.6 |
| Lung Cancer (OD04237-01) | 41.5 | Liver Tissue 6004-N | 12.0 |
| Lung Margin (OD04237-02) | 34.2 | Liver Cancer 6005-T | 12.1 |
| Ocular Mel Met to Liver | 97.3 | Liver Tissue 6005-N | 5.7 |
| (ODO4310) | | | |
| Liver Margin (ODO4310) | 5.0 | Normal Bladder | 38.2 |
| Melanoma Metastasis | 87.7 | Bladder Cancer | 21.3 |
| Lung Margin (OD04321) | 56.3 | Bladder Cancer | 46.0 |
| Normal Kidney | 30.1 | Bladder Cancer (OD04718-01) | 96.6 |
| Kidney Ca, Nuclear grade 2 | 46 7 | Bladder Normal Adjacent | 29.5 |
| (OD04338) | | (OD04718-03) | |
| Kidney Margin (OD04338) | 14.8 | Normal Ovary | 21.5 |
| Kidney Ca Nuclear grade½ | 52.1 | Ovarian Cancer | 73.7 |
| (OD04339) | | | |
| Kidney Margin (OD04338) | 20.3 | Ovarian Cancer (OD04768-07) | 48.3 |
| Kidney Ca, Clear cell type | 49.0 | Ovary Margin (OD04768-08) | 18.8 |
| (OD04340) | | | |
| Kidney Margin (OD04340) | 23.2 | Normal Stomach | 13.9 |
| Kidney Ca, Nuclear grade 3 | 42.6 | Gastric Cancer 9060358 | 6.7 |
| (OD04348) | | | |
| Kidney Margin (OD04348) | 28.9 | Stomach Margin 9060359 | 13.2 |
| Kidney Cancer (OD04622-01) | 50.7 | Gastric Cancer 9060395 | 28.3 |
| Kidney Margin (OD04622-03) | 8.6 | Stomach Margin 9060394 | 18.0 |
| Kidney Cancer (OD04450-01) | 21.8 | Gastric Cancer 9060397 | 45.4 |
| Kidney Margin (OD04450-03) | 18.2 | Stomach Margin 9060396 | 10.4 |
| Kidney Cancer 8120607 | 25.0 | Gastric Cancer 064005 | 48.3 |

TABLE 80
Panel 4D

| | Rel. Exp. (%) | | Rel. Exp. (%) |
|---|---|---|---|
| | Ag2011, Run | | Ag2011, Run |
| Tissue Name | 160997385 | Tissue Name | 160997385 |
| Secondary Th1 act | 4.7 | HUVEC IL-1beta | 2.0 |
| Secondary Th2 act | 6.4 | HUVEC IFN gamma | 4.0 |
| Secondary Tr1 act | 8.6 | HUVEC TNF alpha + IFN | 5.0 |
| | | gamma | |
| Secondary Th1 rest | 0.6 | HUVEC TNF alpha + IL4 | 8.4 |
| Secondary Th2 rest | 1.7 | HUVEC IL-11 | 3.5 |
| Secondary Tr1 rest | 1.7 | Lung Microvascular EC none | 13.0 |
| Primary Th1 act | 14.0 | Lung Microvascular EC | 15.3 |
| | | TNFalpha + IL-1beta | |
| Primary Th2 act | 7.7 | Microvascular Dermal EC | 23.2 |
| | | none | |
| Primary Tr1 act | 12.9 | Microsvasular Dermal EC | 17.3 |
| | | TNFalpha + IL-1beta | |
| Primary Th1 rest | 3.3 | Bronchial epithelium | 4.5 |
| | | TNFalpha + IL1beta | |
| Primary Th2 rest | 2.3 | Small airway epithelium | 16.0 |
| | | none | |
| Primary Tr1 rest | 2.0 | Small airway epithelium | 100.0 |
| | | TNFalpha + IL-1beta | |
| CD45RA CD4 lymphocyte | 6.5 | Coronery artery SMC rest | 15.7 |
| act | | | |
| CD45RO CD4 lymphocyte | 5.3 | Coronery artery SMC | 11.1 |
| act | | TNFalpha +IL-1beta | |
| CD8 lymphocyte act | 3.3 | Astrocytes rest | 25.3 |
| Secondary CD8 lymphocyte | 7.2 | Astrocytes TNFalpha + IL- | 21.6 |
| rest | | 1beta | |
| Secondary CD8 lymphocyte | 3.0 | KU-812 (Basophil) rest | 8.4 |
| act | | | |
| CD4 lymphocyte none | 1.6 | Ku-812 (Basophil) | 39.5 |
| | | PMA/ionomycin | |
| 2ry Th1/Th2/Tr1_anti-CD95 | 0.3 | CCD1106 (Keratinocytes) | 35.1 |
| CH11 | | none | |
| LAK cells rest | 19.1 | CCD1106 (Keratinocytes) | 5.9 |
| | | TNFalpha + IL-1beta | |
| LAK cells IL-2 | 3.1 | Liver cirrhosis | 0.9 |
| LAK cells IL-2 + IL-12 | 6.5 | Lupus kidney | 1.3 |
| LAK cells IL-2 + IFN gamma | 9.8 | NCI-H292 none | 42.3 |
| LAK cells IL-2 + IL-18 | 5.9 | NCI-H292 IL-4 | 90.1 |
| LAK cells PMA/ionomycin | 8.7 | NCI-H292 IL-9 | 58.2 |
| NK Cells IL-2 rest | 1.7 | NCI-H292 IL-13 | 33.9 |
| Two Way MLR 3 day | 9.3 | NCI-H292 IFN gamma | 30.4 |
| Two Way MLR 5 day | 7.4 | HPAEC none | 5.8 |
| Two Way MLR 7 day | 2.0 | HPAEC TNF alpha + IL-1 | 12.9 |
| | | beta | |
| PBMC rest | 1.7 | Lung fibroblast none | 23.8 |
| PBMC PWM | 12.5 | Lung fibroblast TNF alpha + | 10.7 |
| | | IL-1 beta | |
| PBMC PHA-L | 5.4 | Lung fibroblast IL-4 | 59.0 |
| Ramos (B cell) none | 0.5 | Lung fibroblast IL-9 | 40.6 |
| Ramos (B cell) ionomycin | 0.9 | Lung fibroblast IL-13 | 31.0 |
| B lymphocytes PWM | 15.6 | Lung fibroblast IFN gamma | 65.5 |
| B lymphocytes CD40L and | 5.8 | Dermal fibroblast CCD1070 | 37.4 |
| IL-4 | | rest | |
| EOL-1 dbcAMP | 3.5 | Dermal fibroblast CCD1070 | 50.0 |
| | | TNF alpha | |
| EOL-1 dbcAMP | 60.3 | Dermal fibroblast CCD1070 | 19.6 |
| PMA/ionomycin | | IL-1 beta | |
| Dendritic cells none | 17.6 | Dermal fibroblast IFN | 15.0 |
| | | gamma | |
| Dendritic cells LPS | 32.5 | Dermal fibroblast IL-4 | 43.8 |
| Dendritic cells anti-CD40 | 21.0 | IBD Colitis 2 | 0.3 |
| Monocytes rest | 0.1 | IBD Crohn's | 0.8 |
| Monocytes LPS | 8.4 | Colon | 5.3 |
| Macrophages rest | 34.2 | Lung | 15.0 |
| Macrophages LPS | 11.3 | Thymus | 5.8 |
| HUVEC none | 6.5 | Kidney | 11.4 |
| HUVEC starved | 9.3 | | |

Panel 1.3D Summary: Ag1586/Ag2011

Two experiments with the same probe and primer set produce results that are in excellent agreement. NOV12 appears to be expressed largely in cancer cell lines, with highest expression in a melanoma cell line (CTs=26-28). Of note is the expression associated with colon cancer cell lines as well as melanoma cell lines. Thus, the expression of thie gene could be used to distinguish these samples from other samples on the panel. Moreover, therapeutic modulation of this gene, through the use of small molecule drugs, antibodies or protein therapeutics might be of use in the treatment of colon cancer or melanoma.

This gene is modestly expressed in a variety of metabolic tissues including pancreas, adrenal, thyroid, pituitary, fetal liver, and adipose. Thus, this gene product may be an antibody target for the treatment of metabolic disease, including obesity and diabetes, in any or all of these tissues. In addition, NOV12 is differentially expressed in fetal (CT values=26-28) versus adult heart (CT values=31-33), and in fetal (CT values=26-28) versus adult skeletal muscle (CT values=32-33), and may be used to differentiate between the adult and fetal sources of these tissues. Furthermore, the higher levels of expression in the fetal tissues suggest that the SC132340676_A gene product may be involved in the development of heart and skeletal muscle tissue. Thus, therapeutic modulation of the expression or function of the protein encoded by the SC132340676_A gene may be beneficial in the treatment of diseases that result in weak or dystrophic heart or skeletal muscle tissue, including ardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, muscular dystrophy, Lesch-Nyhan syndrome, and myasthenia gravis.

This gene represents a novel protein with homology to a plexin that is expressed at moderate to high levels in all brain regions examined. Plexins act as receptors for semaphorins in the CNS. The interactions of the semaphorins and their receptors are critical for axon guidance. Therefore, this gene product may be useful as a drug target in clinical conditions where axonal growth and/or compensatory synaptogenesis are desireable (spinal cord or head trauma, stroke, or neurodegenerative diseases such as Alzheimer's, Parkinson's, or Huntington's disease).

References:

1. Pasterkamp R J, Ruitenberg M J, Verhaagen J. Semaphorins and their receptors in olfactory axon guidance. Cell Mol Biol (Noisy-le-grand) September 1999; 45(6):763-79

The mammalian olfactory system is capable of discriminating among a large variety of odor molecules and is therefore essential for the identification of food, enemies and mating partners. The assembly and maintenance of olfactory connectivity have been shown to depend on the combinatorial actions of a variety of molecular signals, including extracellular matrix, cell adhesion and odorant receptor molecules. Recent studies have identified semaphorins and their receptors as putative molecular cues involved in olfactory pathfinding, plasticity and regeneration. The semaphorins comprise a large family of secreted and transmembrane axon guidance proteins, being either repulsive or attractive in nature. Neuropilins were shown to serve as receptors for secreted class 3 semaphorins, whereas members of the plexin family are receptors for class 1 and V (viral) semaphorins. The present review will discuss a role for semaphorins and their receptors in the establishment and maintenance of olfactory connectivity.

2. Murakami Y, Suto F, Shimizu M, Shinoda T, Kameyama T, Fujisawa H. Differential expression of plexin-A subfamily members in the mouse nervous system. Dev Dyn March 2001; 220(3):246-58

Plexins comprise a family of transmembrane proteins (the plexin family) which are expressed in nervous tissues. Some plexins have been shown to interact directly with secreted or transmembrane semaphorins, while plexins belonging to the A subfamily are suggested to make complexes with other membrane proteins, neuropilins, and propagate chemorepulsive signals of secreted semaphorins of class 3 into cells or neurons. Despite that much information has been gathered on the plexin-semaphorin interaction, the role of plexins in the nervous system is not well understood. To gain insight into the functions of plexins in the nervous system, we analyzed spatial and temporal expression patterns of three members of the plexin-A subfamily (plexin-A1, -A2, and -A3) in the developing mouse nervous system by in situ hybridization analysis in combination with immunohistochemistry. We show that the three plexins are differentially expressed in sensory receptors or neurons in a developmentally regulated manner, suggesting that a particular plexin or set of plexins is shared by neuronal elements and functions as the receptor for semaphorins to regulate neuronal development.

Panel 2.2 Summary: Ag2011

The expression of NOV12 appears to be highest in a sample derived from a melanoma metastasis. In addition, there is substantial expression in another melanoma sample. These results are in agreement with the results seen in Panel 1.3D, with significant expression detected in melanoma cell lines. Thus, the expression of this gene could be used to distinguish melanoma from other cancer types in this panel. Moreover, therapeutic modulation of this gene, through the use of small molecule drugs, antibodies or protein therapeutics might be of use in the treatment of melanoma.

Panel 2D Summary: Ag1586

The expression of NOV12 is highest in a sample derived from a metastasis of lung cancer. Thus, the expression of this gene could be used to distinguish this sample from the others in the panel. In addition, there is substantial expression in bladder cancer, when compared to its normal adjacent tissue, as well as in two samples of melanoma. Thus, the expression of this gene could be used to distinguish this bladder cancer from its normal adjacent tissue, or these melanomas from other samples. Moreover, therapeutic modulation of this gene, through the use of small molecule drugs, antibodies or protein therapeutics might be of use in the treatment of lung cancer, bladder cancer or melanoma.

Panel 4D Summary: Ag2011

Significant expression of the NOV12 transcript is found in small airway epithelium upon treatment with the pro-inflammatory cytokines TNF-a and IL-1b (CT=26.5), the muco-epidermoid cell line H 292 treated with IL-4 or IL-9, and in lung fibroblasts treated with IFN-g or IL-4. The constitutive expression of this transcript in these tissues is highly up-regulated by pro-inflammatory cytokines or in conditions reflecting a Th2 mediated mechanism. Therefore, modulation of the expression of the protein encoded by this transcript could be useful for the treatment of lung inflammatory diseases that result from infection of the lung (bronchitis, pneumonia) and for the treatment of Th2-mediated lung disease such as asthma or COPD. Significant expression of this transcript is also found in eosinophils upon PMA and ionomycin treatment, conditions that lead to production of eosinophil specific mediators. This production could contribute to the pathologies associated with asthma, other atopic diseases and inflammatory bowel disease. This gene encodes a novel protein with homology to members of the plexin family, a family of transmembrane proteins which act as receptors for semaphorins. In neurons, semaphorins provide essential attractive and repulsive cues that are necessary for axon guidance. The description of the interaction of plexin wih tyrosine kinase in the fetal lung suggests that this protein may play a role not only in morphogenesis but also in proliferation of activation. (See reference below.) Therefore, modulation of the experession of this protein by either antibody or small molecules could be beneficial for the treatment of inflammatory lung, bowel and skin diseases.

Reference:

1. Cell Oct. 1, 1999; 99(1):71-80

Plexins are a large family of receptors for transmembrane, secreted, and GPI-anchored semaphorins in vertebrates.

Tamagnone L, Artigiani S, Chen H, He Z, Ming G I, Song H, Chedotal A, Winberg M L, Goodman C S, Poo M, Tessier-Lavigne M, Comoglio P M.

Institute for Cancer Research and Treatment, University of Torino, Candiolo, Italy. Itamagnone@ircc.unito.it

In Drosophila, plexin A is a functional receptor for semaphorin-1a. Here we show that the human plexin gene family comprises at least nine members in four subfamilies. Plexin-B 1 is a receptor for the transmembrane semaphorin Sema4D (CD 100), and plexin-C1 is a receptor for the GPI-anchored semaphorin Sema7A (Sema-K1). Secreted (class 3) semaphorins do not bind directly to plexins, but rather plexins associate with neuropilins, coreceptors for these semaphorins. Plexins are widely expressed: in neurons, the expression of a truncated plexin-A1 protein blocks axon repulsion by Sema3A. The cytoplasmic domain of plexins associates with a tyrosine kinase activity. Plexins may also act as ligands mediating repulsion in epithelial cells in vitro. We conclude that plexins are receptors for multiple (and perhaps all) classes of semaphorins, either alone or in combination with neuropilins, and trigger a novel signal transduction pathway controlling cell repulsion

PMID: 10520995

### Example 3

SNP Analysis of NOVX Clones

SeqCallingTM Technology: cDNA was derived from various human samples representing multiple tissue types, normal and diseased states, physiological states, and developmental states from different donors. Samples were obtained as whole tissue, cell lines, primary cells or tissue cultured primary cells and cell lines. Cells and cell lines may have been treated with biological or chemical agents that regulate gene expression for example, growth factors, chemokines, steroids. The cDNA thus derived was then sequenced using CuraGen's proprietary SeqCalling technology. Sequence traces were evaluated manually and edited for corrections if appropriate. cDNA sequences from all samples were assembled with themselves and with public ESTs using bioinformatics programs to generate CuraGen's human SeqCalling database of SeqCalling assemblies. Each assembly contains one or more overlapping cDNA sequences derived from one or more human samples. Fragments and ESTs were included as components for an assembly when the extent of identity with another component of the assembly was at least 95% over 50 bp. Each assembly can represent a gene and/or its variants such as splice forms and/or single nucleotide polymorphisms (SNPs) and their combinations.

Variant sequences are included in this application. A variant sequence can include a single nucleotide polymorphism (SNP). A SNP can, in some instances, be referred to as a “cSNP” to denote that the nucleotide sequence containing the SNP originates as a cDNA. A SNP can arise in several ways. For example, a SNP may be due to a substitution of one nucleotide for another at the polymorphic site. Such a substitution can be either a transition or a transversion. A SNP can also arise from a deletion of a nucleotide or an insertion of a nucleotide, relative to a reference allele. In this case, the polymorphic site is a site at which one allele bears a gap with respect to a particular nucleotide in another allele. SNPs occurring within genes may result in an alteration of the amino acid encoded by the gene at the position of the SNP. Intragenic SNPs may also be silent, however, in the case that a codon including a SNP encodes the same amino acid as a result of the redundancy of the genetic code. SNPs occurring outside the region of a gene, or in an intron within a gene, do not result in changes in any amino acid sequence of a protein but may result in altered regulation of the expression pattern for example, alteration in temporal expression, physiological response regulation, cell type expression regulation, intensity of expression, stability of transcribed message.

Method of novel SNP Identification: SNPs are identified by analyzing sequence assemblies using CuraGen's proprietary SNPTool algorithm. SNPTool identifies variation in assemblies with the following criteria: SNPs are not analyzed within 10 base pairs on both ends of an alignment; Window size (number of bases in a view) is 10; The allowed number of mismatches in a window is 2; Minimum SNP base quality (PHRED score) is 23; Minimum number of changes to score an SNP is 2/assembly position. SNPTool analyzes the assembly and displays SNP positions, associated individual variant sequences in the assembly, the depth of the assembly at that given position, the putative assembly allele frequency, and the SNP sequence variation. Sequence traces are then selected and brought into view for manual validation. The consensus assembly sequence is imported into CuraTools along with variant sequence changes to identify potential amino acid changes resulting from the SNP sequence variation. Comprehensive SNP data analysis is then exported into the SNPCalling database.

Method of novel SNP Confirmation: SNPs are confirmed employing a validated method know as Pyrosequencing (Pyrosequencing, Westborough, Mass.). Detailed protocols for Pyrosequencing can be found in: Alderborn et al. Determination of Single Nucleotide Polymorphisms by Real-time Pyrophosphate DNA Sequencing. (2000). Genome Research. 10, Issue 8, August. 1249-1265. In brief, Pyrosequencing is a real time primer extension process of genotyping. This protocol takes double-stranded, biotinylated PCR products from genomic DNA samples and binds them to streptavidin beads. These beads are then denatured producing single stranded bound DNA. SNPs are characterized utilizing a technique based on an indirect bioluminometric assay of pyrophosphate (PPi) that is released from each dNTP upon DNA chain elongation. Following Klenow polymerase-mediated base incorporation, PPi is released and used as a substrate, together with adenosine 5′-phosphosulfate (APS), for ATP sulfurylase, which results in the formation of ATP. Subsequently, the ATP accomplishes the conversion of luciferin to its oxi-derivative by the action of luciferase. The ensuing light output becomes proportional to the number of added bases, up to about four bases. To allow processivity of the method dNTP excess is degraded by apyrase, which is also present in the starting reaction mixture, so that only dNTPs are added to the template during the sequencing. The process has been fully automated and adapted to a 96-well format, which allows rapid screening of large SNP panels. The DNA and protein sequences for the novel single nucleotide polymorphic variants are reported. Variants are reported individually but any combination of all or a select subset of variants are also included. In addition, the positions of the variant bases and the variant amino acid residues are underlined.

Results

Variants are reported individually but any combination of all or a select subset of variants are also included as contemplated NOVX embodiments of the invention.

NOV6 SNP data:

NOV6 has two SNP variants, whose variant positions for their nucleotide and amino acid sequences is numbered according to SEQ ID NOS: 17 and 18, respectively. The nucleotide sequence of the NOV6 variants differs as shown in Table 81.

TABLE 81
cSNP and Coding Variants for NOV6

| NT position | Wild Type | | Amino Acid | Amino Acid |
|---|---|---|---|---|
| of cSNP | NT | Variant NT | position | Change |
| 446 | T | C | No change | No change |
| 553 | A | G | No change | No change |

NOV8 has two SNP variants, whose variant positions for their nucleotide and amino acid sequences is numbered according to SEQ ID NOS: 21 and 22, respectively. The nucleotide sequence of the NOV8 variants differs as shown in Table 82.

TABLE 82
cSNP and Coding Variants for NOV8

| NT Position | Wild Type | | Amino Acid | Amino Acid |
|---|---|---|---|---|
| of cSNP | NT | Variant NT | position | Change |
| 564 | G | A | 109 | G->D |
| 976 | T | G | No change | No change |

NOV9 SNP Data:

NOV 9 has two SNP variants, whose variant positions for their nucleotide and amino acid sequences is numbered according to SEQ ID NO: 23 and 24, respectively. The nucleotide sequence of the NOV9 variants differs as shown in Table 83.

TABLE 83
cSNP and Coding Variants for NOV9

| NT Position | Wild Type | | Amino Acid | Amino Acid |
|---|---|---|---|---|
| of cSNP | NT | Variant NT | position | Change |
| 111 | A | C | No change | No change |
| 200 | A | G | 62 | K→R |

NOV10 SNP Data:

NOV10 has two SNP variants, whose variant positions for their nucleotide and amino acid sequences is numbered according to SEQ ID NOS: 25 and 26, respectively. The nucleotide sequence of the NOV10 variants differs as shown in Table 84.

TABLE 84
cSNP and Coding Variants for NOV10

| NT Position | Wild Type | | Amino Acid | Amino Acid |
|---|---|---|---|---|
| of cSNP | NT | Variant NT | position | Change |
| 2129 | C | T | No change | No change |
| 2450 | T | C | No change | No change |

NOV11 SNP Data:

NOV11a has three SNP variants, whose variant positions for their nucleotide and amino acid sequences is numbered according to SEQ ID NOS: 27 and 28, respectively. The nucleotide sequence of the NOV11a variant differs as shown in Table 85.

TABLE 85
cSNP and Coding Variants for NOV11a

| NT Position | Wild Type | Variant | Amino Acid | Amino Acid |
|---|---|---|---|---|
| of cSNP | NT | NT | position | Change |
| 122 | C | G | No change | No change |
| 208 | G | C | No change | No change |
| 372 | C | T | 97 | P −> L |
| 482 | A | G | 134 | N −> D |

### Example 4

In-frame Cloning

NOV1b

For NOV1b, the cDNA coding for the DOMAIN of NOV1a (CG50718-02) from residues 18 to 917 was targeted for “in-frame” cloning by PCR. The PCR template was based on the previously identified plasmid, when available, or on human cDNA(s).

TABLE 86

Oligonucleotide primers used to clone the target cDNA sequence:

| | |
|---|---|
| Primers | Sequences |

| | | |
|---|---|---|
| F1 | 5′-AGATCTCAGGTAGATGTTTCCAATGTCGTTCC-3′ | (SEQ ID NO:196) |
| R1 | 5′-CTCGAGGCTAGCGTTACATAAGCACTGTATTCAAC-3′ | (SEQ ID NO:197) |

NOV11c

For NOV11c, the cDNA coding for the DOMAIN of NOV11b (CG54503_{—}02) from residues 15 to 238 was targeted for “in-frame” cloning by PCR. The PCR template was based on the previously identified plasmid, when available, or on human cDNA(s).

TABLE 87

Oligonucleotide primers used to clone the target cDNA sequence:

| | |
|---|---|
| Primers | Sequences |

| | | |
|---|---|---|
| F2 | 5′-GGATCC TCCCGCGGGCCAGCGCACTACGAGATGCTGGGTCG-3′ | (SEQ ID NO:198) |
| R1 | 5′-CTCGAGGTCGGGGTAGAT GATGAAGCCGGAGAAGGTGCTGTACTTGTTGG-3′ | (SEQ ID NO:199) |

For downstream cloning purposes, the forward primer includes an in-frame Hind III restriction site and the reverse primer contains an in-frame Xho I restriction site.

Two parallel PCR reactions were set up using a total of 0.5-1.0 ng human pooled cDNAs as template for each reaction. The pool is composed of 5 micrograms of each of the following human tissue cDNAs: adrenal gland, whole brain, amygdala, cerebellum, thalamus, bone marrow, fetal brain, fetal kidney, fetal liver, fetal lung, heart, kidney, liver, lymphoma, Burkitt's Raji cell line, mammary gland, pancreas, pituitary gland, placenta, prostate, salivary gland, skeletal muscle, small Intestine, spleen, stomach, thyroid, trachea, uterus.

When the tissue of expression is known and available, the second PCR was performed using the above primers and 0.5ng-1.0 ng of one of the following human tissue cDNAs:

skeleton muscle, testis, mammary gland, adrenal gland, ovary, colon, normal cerebellum, normal adipose, normal skin, bone marrow, brain amygdala, brain hippocampus, brain substantia nigra, brain thalamus, thyroid, fetal lung, fetal liver, fetal brain, kidney, heart, spleen, uterus, pituitary gland, lymph node, salivary gland, small intestine, prostate, placenta, spinal cord, peripheral blood, trachea, stomach, pancreas, hypothalamus.

The reaction mixtures contained 2 microliters of each of the primers (original concentration: 5 pmol/ul), 1 microliter of 10 mM dNTP (Clontech Laboratories, Palo Alto Calif.) and 1 microliter of 50×Advantage-HF 2 polymerase (Clontech Laboratories) in 50 microliter-reaction volume. The following reaction conditions were used:

PCR condition 1:

| a) | 96° C. | 3 minutes |
|---|---|---|
| b) | 96° C. | 30 seconds denaturation |
| c) | 60° C. | 30 seconds, primer annealing |
| d) | 72° C. | 6 minutes extension |
| Repeat steps b-d 15 times | | |
| e) | 96° C. | 15 seconds denaturation |
| f) | 60° C. | 30 seconds, primer annealing |
| g) | 72° C. | 6 minutes extension |
| Repeat steps e-g 29 times | | |
| e) | 72° C. | 10 minutes final extension |

PCR condition 2:

| a) | 96° C. | 3 minutes |
|---|---|---|
| b) | 96° C. | 15 seconds denaturation |
| c) | 76° C. | 30 seconds, primer annealing, |
| | | reducing the temperature by |
| | | 1° C. per cycle |
| d) | 72° C. | 4 minutes extension |
| Repeat steps b-d 34 times | | |
| e) | 72° C. | 10 minutes final extension |

An amplified product was detected by agarose gel electrophoresis. The fragment was gel-purified and ligated into the pCR2.1 vector (Invitrogen, Carlsbad, Calif.) following the manufacturer's recommendation. Twelve clones per PCR reaction were picked and sequenced. The inserts were sequenced using vector-specific M13 Forward and M13 Reverse primers and the gene-specific primers in Tables 88 and 89.

TABLE 88

Gene-specific Primers

| | | |
|---|---|---|
| NOV | Primers | Sequences |

| | | | |
|---|---|---|---|
| NOV11c | SF1 | GCCCTCCCGGTCCAGGTC | (SEQ ID NO:200) |
| | SF2 | GGCGACGGCACCAGCATGT | (SEQ ID NO:201) |
| | SR1 | GCCTGGCCTGCCCGGTTCT | (SEQ ID NO:202) |
| | SR2 | CATGAGCACGTGGTAAGCG | (SEQ ID NO:203) |

TABLE 89
Gene-specific Primers

| | | |
|---|---|---|
| NOV | Primers | Sequences |

(SEQ ID NO:204)

| | | |
|---|---|---|
| NOV1b | SF1 | GTGCTGGCATTGGAGTGTTTAGTG |

(SEQ ID NO:205)

| | |
|---|---|
| SF2 | ATCAAGCACGTTGACACAGAATGAG |

(SEQ ID NO:206)

| | |
|---|---|
| SF3 | GCATTCACTAACCTAACACCATTTACA |

(SEQ ID NO:207)

| | |
|---|---|
| SF4 | GTTCAGCAGAGATGTCGTCTGACCTTC |

(SEQ ID NO:208)

| | |
|---|---|
| SF5 | GGGATCCTCCAGATCCTGTATTTTT |

(SEQ ID NO:209)

| | |
|---|---|
| SF6 | TGAAGAACACATCAACAACAGACATAA |

(SEQ ID NO:210)

| | |
|---|---|
| SR1 | ACTGTTTTCAGCAGCTACCTTAATTTC |

(SEQ ID NO:211)

| | |
|---|---|
| SR2 | CTTGATGAATGTGTGGTACGCGAT |

(SEQ ID NO:212)

| | |
|---|---|
| SR3 | GTGAATGCAAACTTGAGGTCTTTTGT |

(SEQ ID NO:213)

| | |
|---|---|
| SR4 | CCTCATATAATCCTACCATTGGCTGTACT |

(SEQ ID NO:214)

| | |
|---|---|
| SR5 | GAGGATCCCAGTGTAAAAATACTTCTG |

(SEQ ID NO:215)

| | |
|---|---|
| SR6 | TAGCACTTCATAAGCAATAATGATCCC |

(SEQ ID NO:216)

| | |
|---|---|
| SR7 | TGAGTGTACTAGCAGACACCTCAATGAT |

### OTHER EMBODIMENTS

Although particular embodiments have been disclosed herein in detail, this has been done by way of example for purposes of illustration only, and is not intended to be limiting with respect to the scope of the appended claims, which follow. In particular, it is contemplated by the inventors that various substitutions, alterations, and modifications may be made to the invention without departing from the spirit and scope of the invention as defined by the claims. The choice of nucleic acid starting material, clone of interest, or library type is believed to be a matter of routine for a person of ordinary skill in the art with knowledge of the embodiments described herein. Other aspects, advantages, and modifications considered to be within the scope of the following claims.

What is claimed is:

1. An isolated polypeptide comprising an amino acid sequence selected from the group consisting of:
(a) a mature form of an amino acid sequence selected from the group consisting of SEQ ID NOS: 2, 4, 6, 8, 10, 12, 14, 16, 18, 20, 22, 24, 26, 28, 30, and 32;
(b) a variant of a mature form of an amino acid sequence selected from the group consisting of SEQ ID NOS: 2, 4, 6, 8, 10, 12, 14, 16, 18, 20, 22, 24, 26, 28, 30, and 32, wherein one or more amino acid residues in said variant differs from the amino acid sequence of said mature form, provided that said variant differs in no more than 15% of the amino acid residues from the amino acid sequence of said mature form;
(c) an amino acid sequence selected from the group consisting SEQ ID NOS: 2, 4, 6, 8, 10, 12, 14, 16, 18, 20, 22, 24, 26, 28, 30, and 32; and
(d) a variant of an amino acid sequence selected from the group consisting of SEQ ID NOS: 2, 4, 6, 8, 10, 12, 14, 16, 18, 20, 22, 24, 26, 28, 30, and 32, wherein one or more amino acid residues in said variant differs from the amino acid sequence of said mature form, provided that said variant differs in no more than 15% of amino acid residues from said amino acid sequence.

2 The polypeptide of claim 1, wherein said polypeptide comprises the amino acid sequence of a naturally-occurring allelic variant of an amino acid sequence selected from the group consisting SEQ ID NOS: 2, 4, 6, 8, 10, 12, 14, 16, 18, 20, 22, 24, 26, 28, 30, and 32.

3. The polypeptide of claim 2, wherein said allelic variant comprises an amino acid sequence that is the translation of a nucleic acid sequence differing by a single nucleotide from a nucleic acid sequence selected from the group consisting of SEQ ID NOS: 1, 3, 5, 7, 9, 11, 13, 15, 17, 19, 21, 23, 25, 27, 29, and 31.

4. The polypeptide of claim 1, wherein the amino acid sequence of said variant comprises a conservative amino acid substitution.

5. An isolated nucleic acid molecule comprising a nucleic acid sequence encoding a polypeptide comprising an amino acid sequence selected from the, group consisting of:
(a) a mature form of an amino acid sequence selected from the group consisting of SEQ ID NOS: 2, 4, 6, 8, 10, 12, 14, 16, 18, 20, 22, 24, 26, 28, 30, and 32;
(b) a variant of a mature form of an amino acid sequence selected from the group consisting of SEQ ID NOS: 2, 4, 6, 8, 10, 12, 14, 16, 18, 20, 22, 24, 26, 28, 30, and 32, wherein one or more amino acid residues in said variant differs from the amino acid sequence of said mature form, provided that said variant differs in no more than 15% of the amino acid residues from the amino acid sequence of said mature form;
(c) an amino acid sequence selected from the group consisting of SEQ ID NOS: 2, 4, 6, 8, 10, 12, 14, 16, 18, 20, 22, 24, 26, 28, 30, and 32;
(d) a variant of an amino acid sequence selected from the group consisting SEQ ID NOS: 2, 4, 6, 8, 10, 12, 14, 16, 18, 20, 22, 24, 26, 28, 30, and 32, wherein one or more amino acid residues in said variant differs from the amino acid sequence of said mature form, provided that said variant differs in no more than 15% of amino acid residues from said amino acid sequence;
(e) a nucleic acid fragment encoding at least a portion of a polypeptide comprising an amino acid sequence chosen from the group consisting of SEQ ID NOS: 2, 4, 6, 8, 10, 12, 14, 16, 18, 20, 22, 24, 26, 28, 30, and 32, or a variant of said polypeptide, wherein one or more amino acid residues in said variant differs from the amino acid sequence of said mature form, provided that said variant differs in no more than 15% of amino acid residues from said amino acid sequence; and
(f) a nucleic acid molecule comprising the complement of (a), (b), (c), (d) or (e).

6. The nucleic acid molecule of claim 5, wherein the nucleic acid molecule comprises the nucleotide sequence of a naturally-occurring allelic nucleic acid variant.

7. The nucleic acid molecule of claim 5, wherein the nucleic acid molecule encodes a polypeptide comprising the amino acid sequence of a naturally-occurring polypeptide variant.

8. The nucleic acid molecule of claim 5, wherein the nucleic acid molecule differs by a single nucleotide from a nucleic acid sequence selected from the group consisting of SEQ ID NOS: 1, 3, 5, 7, 9, 11, 13, 15, 17, 19, 21, 23, 25, 27, 29, and31.

9. The nucleic acid molecule of claim 5, wherein said nucleic acid molecule comprises a nucleotide sequence selected from the group consisting of:
(a) a nucleotide sequence selected from the group consisting of SEQ ID NOS: 1, 3, 5, 7, 9, 11, 13, 15, 17, 19, 21, 23, 25, 27, 29, and31;
(b) a nucleotide sequence differing by one or more nucleotides from a nucleotide sequence selected from the group consisting of SEQ ID NOS: 1, 3, 5, 7, 9, 11, 13, 15, 17, 19, 21, 23, 25, 27, 29, and 31, provided that no more than 20% of the nucleotides differ from said nucleotide sequence;
(c) a nucleic acid fragment of (a); and
(d) a nucleic acid fragment of (b).

10. The nucleic acid molecule of claim 5, wherein said nucleic acid molecule hybridizes under stringent conditions to a nucleotide sequence chosen from the group consisting SEQ ID NOS: 1, 3, 5, 7, 9, 11, 13, 15, 17, 19, 21, 23, 25, 27, 29, and 31, or a complement of said nucleotide sequence.

11. The nucleic acid molecule of claim 5, wherein the nucleic acid molecule comprises a nucleotide sequence selected from the group consisting of:
(a) a first nucleotide sequence comprising a coding sequence differing by one or more nucleotide sequences from a coding sequence encoding said amino acid sequence, provided that no more than 20% of the nucleotides in the coding sequence in said first nucleotide sequence differ from said coding sequence;
(b) an isolated second polynucleotide that is a complement of the first polynucleotide;
and
(c) a nucleic acid fragment of (a) or (b).

12. A vector comprising the nucleic acid molecule of claim 11.

13. The vector of claim 12, further comprising a promoter operably-linked to said nucleic acid molecule.

14. A cell comprising the vector of claim 12.

15. An antibody that binds immunospecifically to the polypeptide of claim 1.

16. The antibody of claim 15, wherein said antibody is a monoclonal antibody.

17. The antibody of claim 15, wherein the antibody is a humanized antibody.

18. A method for determining the presence or amount of the polypeptide of claim 1 in a sample, the method comprising:
(a) providing the sample;
(b) contacting the sample with an antibody that binds immunospecifically to the polypeptide; and
(c) determining the presence or amount of antibody bound to said polypeptide, thereby determining the presence or amount of polypeptide in said sample.

19. A method for determining the presence or amount of the nucleic acid molecule of claim 5 in a sample, the method comprising:
(a) providing the sample;
(b) contacting the sample with a probe that binds to said nucleic acid molecule; and
(c) determining the presence or amount of the probe bound to said nucleic acid molecule,
thereby determining the presence or amount of the nucleic acid molecule in said sample.

20. The method of claim 19 wherein presence or amount of the nucleic acid molecule is used as a marker for cell or tissue type.

21. The method of claim 20 wherein the cell or tissue type is cancerous.

22. A method of identifying an agent that binds to a polypeptide of claim 1, the method comprising:
(a) contacting said polypeptide with said agent; and
(b) determining whether said agent binds to said polypeptide.

23. The method of claim 22 wherein the agent is a cellular receptor or a downstream effector.

24. A method for identifying an agent that modulates the expression or activity of the polypeptide of claim 1, the method comprising:
(a) providing a cell expressing said polypeptide;
(b) contacting the cell with said agent, and
(c) determining whether the agent modulates expression or activity of said polypeptide,
whereby an alteration in expression or activity of said peptide indicates said agent modulates expression or activity of said polypeptide.

25. A method for modulating the activity of the polypeptide of claim 1, the method comprising contacting a cell sample expressing the polypeptide of said claim with a compound that binds to said polypeptide in an amount sufficient to modulate the activity of the polypeptide.

26. A method of treating or preventing a NOVX-associated disorder, said method comprising administering to a subject in which such treatment or prevention is desired the polypeptide of claim 1 in an amount sufficient to treat or prevent said NOVX-associated disorder in said subject.

27. The method of claim 26 wherein the disorder is selected from the group consisting of cardiomyopathy and atherosclerosis.

28. The method of claim 26 wherein the disorder is related to cell signal processing and metabolic pathway modulation.

29. The method of claim 26, wherein said subject is a human.

30. A method of treating or preventing a NOVX-associated disorder, said method comprising administering to a subject in which such treatment or prevention is desired the nucleic acid of claim 5 in an amount sufficient to treat or prevent said NOVX-associated disorder in said subject.

31. The method of claim 30 wherein the disorder is selected from the group consisting of cardiomyopathy and atherosclerosis.

32. The method of claim 30 wherein the disorder is related to cell signal processing and metabolic pathway modulation.

33. The method of claim 30, wherein said subject is a human.

34. A method of treating or preventing a NOVX-associated disorder, said method comprising administering to a subject in which such treatment or prevention is desired the antibody of claim 15 in an amount sufficient to treat or prevent said NOVX-associated disorder in said subject.

35. The method of claim 34 wherein the disorder is diabetes.

36. The method of claim 34 wherein the disorder is related to cell signal processing and metabolic pathway modulation.

37. The method of claim 34, wherein the subject is a human.

38. A pharmaceutical composition comprising the polypeptide of claim 1 and a pharmaceutically-acceptable carrier.

39. A pharmaceutical composition comprising the nucleic acid molecule of claim 5 and a pharmaceutically-acceptable carrier.

40. A pharmaceutical composition comprising the antibody of claim 15 and a pharmaceutically-acceptable carrier.

41. A kit comprising in one or more containers, the pharmaceutical composition of claim 38.

42. A kit comprising in one or more containers, the pharmaceutical composition of claim 39.

43. A kit comprising in one or more containers, the pharmaceutical composition of claim 40.

44. A method for determining the presence of or predisposition to a disease associated with altered levels of the polypeptide of claim 1 in a first mammalian subject, the method comprising:
(a) measuring the level of expression of the polypeptide in a sample from the first mammalian subject; and
(b) comparing the amount of said polypeptide in the sample of step (a) to the amount of the polypeptide present in a control sample from a second mammalian subject known not to have, or not to be predisposed to, said disease;
wherein an alteration in the expression level of the polypeptide in the first subject as compared to the control sample indicates the presence of or predisposition to said disease.

45. The method of claim 44 wherein the predisposition is to a cancer.

46. A method for determining the presence of or predisposition to a disease associated with altered levels of the nucleic acid molecule of claim 5 in a first mammalian subject, the method comprising:
(a) measuring the amount of the nucleic acid in a sample from the first mammalian subject; and
(b) comparing the amount of said nucleic acid in the sample of step (a) to the amount of the nucleic acid present in a control sample from a second mammalian subject known not to have or not be predisposed to, the disease;
wherein an alteration in the level of the nucleic acid in the first subject as compared to the control sample indicates the presence of or predisposition to the disease.

47. The method of claim 46 wherein the predisposition is to a cancer.

48. A method of treating a pathological state in a mammal, the method comprising administering to the mammal a polypeptide in an amount that is sufficient to alleviate the pathological state, wherein the polypeptide is a polypeptide having an amino acid sequence at least 95% identical to a polypeptide comprising an amino acid sequence of at least one of SEQ ID NOS: 2, 4, 6, 8, 10, 12, 14, 16, 18, 20, 22, 24, 26, 28, 30, and 32, or a biologically active fragment thereof.

49. A method of treating a pathological state in a mammal, the method comprising administering to the mammal the antibody of claim 15 in an amount sufficient to alleviate the pathological state.
